(19) United States
(12) Patent Application Publication
(10) Pub. No.: US 2025/0320213 A1
(43) Pub. Date: Oct. 16, 2025

(54) Nitrogen-Containing Heterocyclic Compounds

(71) Applicant: Pfizer Inc., New York, NY (US)

(72) Inventors: Chulho Choi, Mystic, CT (US); Andrew Fensome, Harvard, MA (US); Kentaro Futatsugi, Sharon, MA (US); Brian Stephen Gerstenberger, Boston, MA (US); Mohammad Mehdi Ghahremanpour, Manchester, NH (US); David Hepworth, Concord, MA (US); Kevin Michael Knopf, Groton, CT (US); Daniel Wei-Shung Kung, Salem, CT (US); Fang-Yu Lin, Needham, MA (US); Ellene Hera Mashalidis, Salem, CT (US); Sarah Jane Mear, East Lyme, CT (US); Mihir Dineshkumar Parikh, East Greenwich, RI (US); Ian Brooks Perry, Pelham, NH (US); Daniel Jonathan Smaltz, Cambridge, MA (US); Jianmin Sun, East Lyme, CT (US); Benjamin Aaron Thuma, Old Lyme, CT (US); Tao Wang, Sudbury, MA (US); Ian William Windsor, Mystic, CT (US)

(73) Assignee: Pfizer Inc., New York, NY (US)

(21) Appl. No.: 19/175,759

(22) Filed: Apr. 10, 2025

Related U.S. Application Data
(60) Provisional application No. 63/633,369, filed on Apr. 12, 2024.
(60) Provisional application No. 63/757,828, filed on Feb. 13, 2025.
(60) Provisional application No. 63/774,036, filed on Mar. 18, 2025.

(51) Int. Cl. C07D 471/14; A61K 31/416; A61K 31/437; A61K 31/444; A61K 31/4545; A61K 31/4709; A61K 31/496; A61K 31/4985; A61K 31/506; A61K 31/513; A61K 31/519; A61K 31/5377; A61K 31/5386; A61K 31/551; A61K 31/553; C07B 59/00; C07D 231/56; C07D 471/04; C07D 471/18; C07D 487/04; C07D 495/04; C07D 498/04; C07D 498/18; C07D 519/00

(52) U.S. Cl. CPC C07D 471/14; A61K 31/416; A61K 31/437; A61K 31/444; A61K 31/4545; A61K 31/4709; A61K 31/496; A61K 31/4985; A61K 31/506; A61K 31/513; A61K 31/519; A61K 31/5377; A61K 31/5386; A61K 31/551; A61K 31/553; C07B 59/002; C07D 231/56; C07D 471/04; C07D 471/18; C07D 487/04; C07D 495/04; C07D 498/04; C07D 498/18; C07D 519/00

(57) ABSTRACT

The disclosure relates to compounds of the disclosure and pharmaceutically acceptable salts thereof to their use in medicine; to compositions containing them; to processes for their preparation; and to intermediates used in such processes. The compounds of the disclosure may be useful in the treatment, prevention, suppression and amelioration of disease(s) such as atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa. The compounds of the disclosure may be useful in the treatment, prevention, suppression and amelioration of a dermatological condition or a respiratory condition.

### BACKGROUND

The present disclosure relates to novel compounds. The disclosure also relates to the preparation of the compounds and intermediates used in the preparation, compositions containing the compounds, and uses of the compounds including their use as a STAT6 inhibitor.

STAT6 is a member of the Signal Transducer and Activator of Transcription (STAT) family of proteins consisting of transcription factors that impact cell processes including differentiation, survival, proliferation, and functional activation [Levy, D E and Darnell, JE. STATs: transcriptional control and biological impact. 2002. Nat Rev Mol Cell Biol. 3(9):651-62]. The STAT family consists of seven members: STAT1, STAT2, STAT3, STAT4, STAT5a, STAT5b, and STAT6.

STAT family proteins are downstream targets of the Janus kinase (JAK) family kinases, which contribute to signal transduction from a variety of cytokines including IL-2, IL-5, GM-CSF, IL-10, IL-12, IL-23, as well as IL-4 and IL-13. Cytokines IL-4 and IL-13 have been demonstrated to signal through STAT6 activation [Kaplan, M H et al. 1996. Stat6 is required for mediating responses to IL-4 and for development of Th2 cells. Immunity. 4: 313-319]. The pathogenic activity of IL-4 and IL-13 cytokines is consistent with efficacy that has been observed with JAK inhibitors, which block signaling of IL-4 and IL-13 as well as signaling of additional inflammatory cytokines [Simpson, E L et al. 2020. Efficacy and safety of abrocitinib in adults and adolescents with moderate-to-severe atopic dermatitis (JADE MONO-1): a multicentre, double-blind, randomised, placebo-controlled, phase 3 trial. Lancet. 396(10246): 255-266; Guttman-Yassky, E et al. 2021. Once-daily upadacitinib versus placebo in adolescents and adults with moderate-to-severe atopic dermatitis (Measure Up 1 and Measure Up 2): results from two replicate double-blind, randomised controlled phase 3 trials. Lancet. 397(10290): 2151-2168].

Despite the effectiveness of known therapeutics, an unmet need remains for safe and effective therapeutics for numerous diseases characterized by inflammatory responses, that address a broad range of pathogenic mechanisms.

### BRIEF SUMMARY

The present disclosure provides, in part, compounds and pharmaceutically acceptable salts thereof. Such compounds may inhibit the activity of STAT6 and may be useful in the treatment, prevention, suppression, and/or amelioration of disease(s), disorders and conditions mediated by STAT6. Also provided are pharmaceutical compositions, comprising the compounds or salts, alone or in combination with additional therapeutic agents. The present disclosure also provides, in part, methods for preparing such compounds, pharmaceutically acceptable salts and compositions of the disclosure, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

In an aspect, a compound of the disclosure has the Formula I or a pharmaceutically acceptable salt thereof:

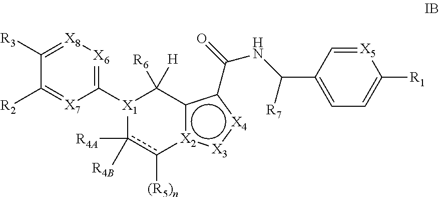

where the variables are defined herein.

In an aspect, a compound of the disclosure has the Formula IA or a pharmaceutically acceptable salt thereof:

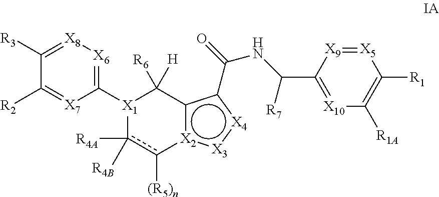

where the variables are defined herein.

In an aspect, a compound of the disclosure has the Formula IB or a pharmaceutically acceptable salt thereof:

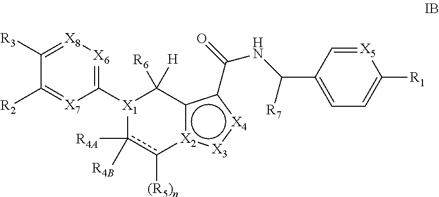

where the variables are defined herein.

In an aspect, the disclosure relates to a pharmaceutical composition comprising a compound of the disclosure, or a pharmaceutically acceptable salt thereof, and at least one pharmaceutically acceptable excipient.

In another aspect, the disclosure relates to a method for treating atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa comprising administering to a subject in need thereof a therapeutically effective amount of the compound of the disclosure.

In another aspect, the disclosure relates to the compound of the disclosure or a pharmaceutically acceptable salt thereof for use as a medicament; or for use in the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa; or for use in the treatment of at least one of a dermatological condition or a respiratory condition.

It is to be understood that both the foregoing general description and the following detailed description are exemplary and explanatory only and are not restrictive of the disclosure, as claimed.

### DETAILED DESCRIPTION

The present disclosure may be understood more readily by reference to the following detailed description and the Examples included herein. It is to be understood that this disclosure is not limited to specific synthetic methods of making that may of course vary. It is to be also understood that the terminology used herein is for the purpose of describing specific aspects only and is not intended to be limiting.

In an aspect, a compound of Formula I or a pharmaceutically acceptable salt thereof:

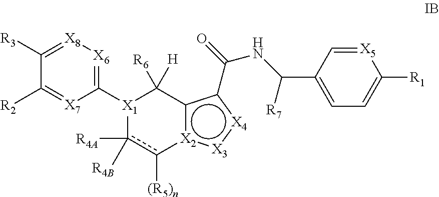

wherein
X₁ is CH, CNH₂, or N;
X₂ is C or N;
X₃ is CR₁₃, N, NR₁₃, O, or S;
X₄ is CH, N, or NR₁₃;
X₅ is CR_{1B} or N;
X₆ is CH or N;
X₇ is CH, N, or CF;
X₈ is CH or N;
X₉ is CR_{1B} or N;
X₁₀ is CR_{1B} or N;
R₁ is —NHR₈, —OH, —C₂₋₅ heterocycle, or —C₁₋₃ alkyl; wherein the —C₂₋₅ heterocycle of R₁ is optionally substituted with one, two, or three of —C₁₋₃ alkyl, —C₁₋₃ oxoalkyl, —C₃₋₅ heterocycloalkyl, —C₃₋₅ hydroxyheterocycloalkyl, —C₃₋₅ aminoheterocycloalkyl, or oxo; wherein the —C₁₋₃ alkyl of R₁ is optionally substituted with one, two, or three of oxo, —NR_{10A}R₁₁, or —NR₁₀R₁₁;
R_{1A} is H, halogen, or —CH₃; or
R₁ and R_{1A} form a C₅₋₇ heterocycloalkyl fused to Ring D or a C₅₋₇ heteroaryl fused to Ring D; wherein the C₅₋₇ heterocycloalkyl or the C₅₋₇ heteroaryl is optionally substituted with oxo;
each R_{1B} independently is H, —CH₃, F, C, or methoxy;
R₂ is H, —C₁₋₃ alkyl, —C₁₋₃ alkoxy, —C₁₋₃ fluoroalkyl, or halogen;
R₃ is —C₁₋₃ alkyl, —C₂₋₁₀ heterocycle, —P(═O)(CH₃)₂, —S(═O)CH₃, —NH—S(═O)₂CH₃, or —NH—C(═O)—C₁₋₃ alkyl; wherein the —C₁₋₃ alkyl of R₃ is optionally substituted with one, two, or three of oxo, halogen, —C₃₋₆ cycloalkyl, —OH, —NR₁₂R_{12A}, or cyano; wherein the —C₂₋₁₀ heterocycle is optionally substituted with one, two, or three of —OH, halogen, —C₁₋₃ hydroxyalkyl, —C₁₋₃ alkoxy, —C₁₋₃ alkyl, —C₁₋₃ fluoroalkyl, cyclopropyl, or oxo; or
R₂ and R₃ form a C₃₋₆ heterocycle fused to Ring A optionally substituted with one, two, or three of —C₁₋₃ alkyl, —C₂₋₃ oxoalkyl, or oxo;
R_{4A} is H, —OH, —C₁₋₃ fluoroalkyl, or —C₁₋₃ alkyl;
R_{4B} is H or is absent; or
R_{4A} and R_{4B} form cyclopropyl;
each R₅ independently is H, halogen, —OH, cyclopropyl, —C₁₋₃ fluoroalkyl, or —C₁₋₃ alkyl; or two R₅ groups form cyclopropyl;
R₆ is H, —OH, or —C₁₋₃ alkyl; or
R_{4A} and R₆ or one R₅ and R₆ form a C₁₋₃ alkyl bridge or a C₁₋₃ heteroalkyl bridge; or
R_{4B} and one R₅ form a C₃₋₅ cycloalkyl fused to Ring B; R₇ is H, —C₁₋₃ alkyl, or —C₁₋₃ hydroxyalkyl;
R₈ is H, —C₁₋₃ alkyl, —SO₂CH₃, or a —C₃₋₄ heterocycle; wherein the —C₁₋₃ alkyl of R₃ is optionally substituted with one, two, or three of oxo, —C₃₋₉ heterocycloalkyl, —C₁₋₃ alkoxy, cyanoimine, or —NR₉R₁₀; wherein the —C₃₋₄ heterocycle of R₃ is optionally substituted with one, two, or three of oxo, halogen, —C₀₋₁ alkylene-NR₁₀R₁₁, —OH, —C₁₋₃ hydroxyalkyl, —C₁₋₃ alkoxy optionally substituted with —NR₁₀R₁₁, —C₁₋₃ alkyl-C₁₋₃ alkoxy, —C₁₋₃ oxoalkyl, or —C₁₋₃ alkyl; wherein the —C₃₋₉ heterocycloalkyl of R₃ is optionally substituted with one, two, or three of —C₁₋₃ alkyl, —OH, —C₁₋₃ hydroxyalkyl, —O—C₁₋₃ hydroxyalkyl, —C₁₋₃ alkoxy, —C₁₋₃ alkyl-C₁₋₃ alkoxy, halogen, —C₁₋₃ fluoroalkyl, —C₁₋₃ fluoroalkoxy, cyano, —C₁₋₃ cyanoalkyl, —C₀₋₁ alkylene-C₃₋₅ heterocycloalkyl, —O—C₃₋₅ heterocycloalkyl, or —C₀₋₁ alkylene-NR₁₀R₁₁;
R₉ is H, —C₁₋₄ alkyl, —C₁₋₄ hydroxyalkyl, —C₁₋₃ fluoroalkyl, —C₃₋₅ cycloalkyl, -methylene-phenylene-NH—C(═O)—NR₁₀R₁₁, or —C₃₋₅ heterocycloalkyl; wherein the —C₁₋₄ alkyl of R₉ is optionally substituted with one, two, or three of —C₃₋₅ cycloalkyl, —C₃₋₅ heterocycloalkyl, or methoxy; wherein the —C₃₋₅ cycloalkyl of R₉ is optionally substituted with one or two of —OH or —C₁₋₃ alkyl;
each R₁₀ independently is H or —C₁₋₄ alkyl; or
R_{10A} is —C₀₋₁ alkylene-C₃₋₅ heterocycloalkyl optionally substituted with —C₁₋₃ alkyl;
each R₁₁ independently is H or —C₁₋₃ alkyl;
R₁₂ is H, —C₁₋₃ alkyl, —C₁₋₃ hydroxyalkyl, —(CH₂)₀₋₂—C₃₋₅ heterocycloalkyl, —(CH₂)₁₋₂—O—C₃₋₅ heterocycloalkyl, -methylene-C(═O)—NR₁₀R₁₁, or -methylene-phenylene-NH—C(═O)—NR₁₀R₁₁;
wherein the —(CH₂)₀₋₂—C₃₋₅ heterocycloalkyl or the —(CH₂)₁₋₂—O—C₃₋₅ heterocycloalkyl of R₁₂ is optionally substituted with —C₁₋₄ alkyl, —C₁₋₃ alkyl-C₁₋₃ alkoxy, —(CH₂)₀₋₁-phenyl, halogen, —C₁₋₃ fluoroalkyl, or —(CH₂)₀₋₁—C₃₋₅ heterocycloalkyl; wherein the —C₁₋₃ alkyl of R₁₂ is optionally substituted with one, two, or three of oxo or NR₁₀R₁₁;
R_{12A} is H or —C₁₋₄ alkyl; or
R₁₂ and R_{12A} form a C₃₋₁₀ heterocycle optionally substituted with one, two, or three of —OH, —C₁₋₃ alkyl, oxo, halogen, —C₂₋₃ oxoalkyl, —C₁₋₃ alkoxy, —C₁₋₃ hydroxyalkyl, —C₁₋₃ alkyl-C₁₋₃ alkoxy, cyano, —C₃₋₆ cycloalkyl, —S(═O)₂CH₃, —S(═O)₂CH₂CH₃, —C(═O)—NR₁₀R₁₁, or —NHC(═O)CH₃;
R₁₃ is H, —C₁₋₆ alkyl, —C₁₋₄ hydroxyalkyl, —C₁₋₃ fluoroalkyl, —C₁₋₃ alkyl-C₁₋₃ alkoxy, —C₁₋₄ cyanoalkyl, —C₃₋₅ cycloalkyl, —C₂₋₄ oxoalkyl, or —C₃₋₅ heterocycloalkyl; wherein the —C₁₋₆ alkyl of R₁₃ is optionally substituted with one, two, or three of oxo, fluoro, —NH₂, C₃₋₆ cycloalkyl, or methoxy; wherein the —C₃₋₅ cycloalkyl or the —C₃₋₆ cycloalkyl of R₁₃ is optionally substituted with one, two, or three of —C₁₋₃ alkyl, —C₁₋₃ fluoroalkyl, or halogen;
R₁₄ is H; or
X₄ is NR₁₃ and R₁₃ and R₁₄ form a C₄₋₅ heterocycle fused to Ring C;
n is 1 or 2;
m is 0 or 1;
wherein if X₃ is CR₁₃ or NR₁₃ then X₄ is CH or N, and if X₄ is NR₁₃ then X₃ is N, O, or S;
wherein the respective heterocycles each independently include 1 to 4 heteroatoms including at least one of N, O, or S.

In an aspect, a compound of Formula IA or a pharmaceutically acceptable salt thereof:

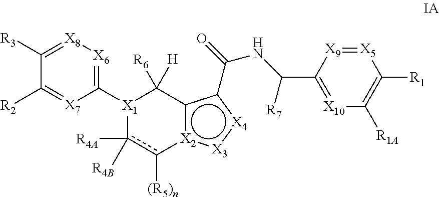

wherein
X₁ is CH, CNH₂, or N;
X₂ is C or N;
X₃ is CR₁₃, N, NR₁₃, O, or S;
X₄ is CH, N, or NR₁₃;
X₅ is CH, CR_{1B}, or N;
X₆ is CH or N;
X₇ is CH, N, or CF;
X₈ is CH or N;
X₉ is CH, CR_{1B}, or N;
X₁₀ is CH, CR_{1B}, or N;
R₁ is —NHR₈, —OH, —C₂₋₅ heterocycle, or —C₁₋₃ alkyl; wherein the —C₂₋₅ heterocycle of R₁ is optionally substituted with one, two, or three of —C₁₋₃ alkyl, —C₁₋₃ oxoalkyl, —C₃₋₅ heterocycloalkyl, —C₃₋₅ hydroxyheterocycloalkyl, —C₃₋₅ aminoheterocycloalkyl, or oxo; wherein the —C₁₋₃ alkyl of R₁ is optionally substituted with one, two, or three of oxo or —NR₁₀R₁₁;
R_{1A} is H or —CH₃; or
R₁ and R_{1A} form a C₆₋₈ fused heterocycloalkyl or a C₆₋₈ fused heteroaryl; wherein the C₆₋₈ fused heterocycloalkyl or the C₆₋₈ fused heteroaryl is optionally substituted with oxo;
each R_{1B} independently is H, —CH₃, F, Cl, or methoxy;
R₂ is H, —C₁₋₃ alkyl, —C₁₋₃ fluoroalkyl, or halogen;
R₃ is —C₁₋₃ alkyl, —C₂₋₁₀ heterocycle, or —NH—C(═O)—C₁₋₃ alkyl; wherein the —C₁₋₃ alkyl of R₃ is optionally substituted with one or more of oxo, —OH, —NR₁₂R_{12A}, —C₅₋₈ heterocycle, or cyano; wherein the —C₂₋₁₀ heterocycle is optionally substituted with one, two, or three of —OH, halogen, —C₁₋₃ hydroxyalkyl, —C₁₋₃ alkyl, cyclopropyl, or oxo; or
R₂ and R₃ form a C₆₋₁₀ fused heterocycle optionally substituted with one, two, or three of —C₁₋₃ alkyl, —C₂₋₃ oxoalkyl, or oxo;
R_{4A} is H, —OH, or —C₁₋₃ alkyl;
R_{4B} is H or is absent;
each R₅ independently is H, halogen, —OH, cyclopropyl, or —C₁₋₃ alkyl; or two R₅ groups form cyclopropyl;
R₆ is H, —OH, or —C₁₋₃ alkyl; or
R_{4A} and R₆ or R₅ and R₆ form a C₁₋₃ alkyl bridge or a C₁₋₃ heteroalkyl bridge;
R₇ is H, —C₁₋₃ alkyl, or —C₁₋₃ hydroxyalkyl;
R₈ is H, —C₁₋₃ alkyl, —SO₂CH₃, or a —C₃₋₄ heterocycle; wherein the —C₁₋₃ alkyl of R₃ is optionally substituted with one, two, or three of oxo, —C₃₋₇ heterocycloalkyl, —C₃₋₇ heterocycloalkyl substituted with methyl, alkoxy, cyanoimine, or —NR₉R₁₀; wherein the —C₃₋₄ heterocycle is optionally substituted with one, two, or three of oxo, halogen, —C₀₋₁ alkylene-NR₁₄R₁₁, —OH, —C₁₋₃ hydroxyalkyl, —C₁₋₃ alkoxy optionally substituted with —NR₁₀R₁₁, —C₁₋₃ alkoxy-C₁₋₃ alkyl, —C₁₋₃ oxoalkyl, or —C₁₋₃ alkyl;
R₉ is H, —C₁₋₄ alkyl, —C₁₋₄ hydroxyalkyl, —C₁₋₃ fluoroalkyl, —C₃₋₅ cycloalkyl, or —C₃₋₅ heterocycloalkyl; wherein the —C₁₋₄ alkyl of R₉ is optionally substituted with one, two, or three of cyclopropyl, —C₃₋₅ heterocycloalkyl, or methoxy; wherein the —C₃₋₅ cycloalkyl of R₉ is optionally substituted with —C₁₋₃ alkyl;
each R₁₀ independently is H or —C₁₋₄ alkyl; or
if the —C₁₋₃ alkyl of R₃ is substituted with —NR₉R₁₀, then the corresponding R₉ and R₁₀ are optionally combined to form a C₃₋₇ heterocycloalkyl optionally substituted with at least one of methoxy, OH, —C₁₋₃ alkyl, halogen, —NH₂, —NHCH₃, or —N(CH₃)₂;
each R₁₁ independently is H or —C₁₋₃ alkyl;
R₁₂ is H, —C₁₋₃ alkyl, —(CH₂)₀₋₁—C₃₋₅ heterocycloalkyl, or -methylene-phenylene-NH—C—NR₁₀R₁₁; wherein the —(CH₂)₀₋₁—C₃₋₅ heterocycloalkyl is optionally substituted with —C₁₋₄ alkyl;
R_{12A} is H or —C₁₋₄ alkyl; or
R₁₂ and R_{12A} form a fused C₅₋₁₀ heterocycloalkyl optionally substituted with —C₁₋₃ alkyl;
R₁₃ is H, —C₁₋₄ alkyl, —C₁₋₄ hydroxyalkyl, —C₁₋₃ fluoroalkyl, —C₀₋₁-C₃₋₅ cycloalkyl, —C₂₋₄ oxoalkyl, or —C₃₋₅ heterocycloalkyl; wherein the —C₁₋₄ alkyl of R₁₃ is optionally substituted with one, two, or three of cyclopropyl or methoxy; wherein the —C₃₋₅ cycloalkyl of R₁₃ is optionally substituted with —C₁₋₃ alkyl;
n is 1 or 2;
wherein the respective heterocycles each independently include 1 to 3 heteroatoms including at least one of N, O, or S;
wherein at least one of X₂ is N or X₃ is N, NH, or NR₁₃, and X₄ is N or NR₁₃; or if X₃ is S or O, then X₂ is C.

In an aspect, a compound has the Formula IB or a pharmaceutically acceptable salt thereof:

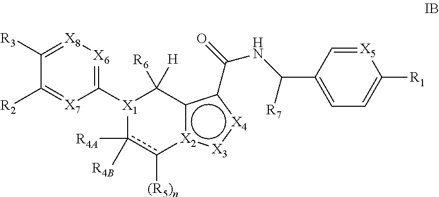

wherein
X₁ is CH, CNH₂, or N;
X₂ is C or N;
X₃ is CR₁₃, N, NR₁₃, O, or S;
X₄ is CH, N, or NR₁₃;
X₅ is CH or N;
X₆ is CH or N;
X₇ is CH, N, or CF;
X₈ is CH or N;
with the proviso that at least one of X₂ is N or X₃ is N, NH, or NR₁₃, and X₄ is N or NR₁₃; or if X₃ is S or O, then X₂ is C;
R₁ is —NHR₈, OH, —C₂₋₅ heterocycle, or —C₁₋₃ alkyl; wherein the —C₂₋₅ heterocycle of R₁ is optionally substituted with one or more of —C₁₋₃ alkyl, —C₁₋₃ oxoalkyl, —C₃₋₅ heterocycloalkyl, —C₃₋₅ hydroxyheterocycloalkyl, —C₃₋₅ aminoheterocycloalkyl, or oxo; wherein the —C₁₋₃ alkyl of R₁ is optionally substituted with one or more of oxo or —NR₁₀R₁₁;
R₂ is H, —C₁₋₃ alkyl, —C₁₋₃ fluoroalkyl, or halogen;
R₃ is —C₁₋₃ alkyl, —C₂₋₁₀ heterocycle, or —NH—C(═O)—C₁₋₃ alkyl; wherein the —C₁₋₃ alkyl of R₃ is optionally substituted with one or more of oxo, —OH, —NR₁₂R_{12A}, —C₅₋₈ heterocycle, or cyano;
wherein the —C₂₋₁₀ heterocycle is optionally substituted with one or more of —OH, halogen, —C₁₋₃ hydroxyalkyl, —C₁₋₃ alkyl, cyclopropyl, or oxo; or
R₂ and R₃ form a C₆₋₁₀ fused heterocycloalkyl optionally substituted with one or more of —C₁₋₃ alkyl, —C₂₋₃ oxoalkyl, or oxo;
R_{4A} is H, —OH, or —C₁₋₃ alkyl;
R_{4B} is H or is absent;
each R₅ independently is H, —OH, halogen, or —C₁₋₃ alkyl; or two R₅ groups form cyclopropyl;
R₆ is H, —OH, or —C₁₋₃ alkyl; or
R_{4A} and R₆ or R₅ and R₆ form a C₁₋₃ alkyl bridge or a C₁₋₃ heteroalkyl bridge;
R₇ is H, —C₁₋₃ alkyl, or —C₁₋₃ hydroxyalkyl;
R₈ is H, —C₁₋₃ alkyl, or a —C₃₋₄ heterocycle; wherein the —C₁₋₃ alkyl of R₈ is optionally substituted with one or more of oxo or —NR₉R₁₀; wherein the —C₃₋₄ heterocycle is optionally substituted with one or more of oxo or —C₁₋₃ alkyl;
R₉ is H, —C₁₋₄ alkyl, —C₁₋₄ hydroxyalkyl, —C₁₋₃ fluoroalkyl, —C₃₋₅ cycloalkyl, or —C₃₋₅ heterocycloalkyl; wherein the —C₁₋₄ alkyl of R₉ is optionally substituted with one or more of cyclopropyl, —C₃₋₅ heterocycloalkyl, or methoxy; wherein the —C₃₋₅ cycloalkyl of R₉ is optionally substituted with —C₁₋₃ alkyl;
each R₁₀ independently is H or —C₁₋₄ alkyl; or
if the —C₁₋₃ alkyl of R₈ is substituted with —NR₉R₁₀, then the corresponding R₉ and R₁₀ are optionally combined to form a C₃₋₆ heterocycloalkyl optionally substituted with at least one of methoxy, OH, —C₁₋₃ alkyl, or —N(CH₃)₂;
each R₁₁ independently is H or —C₁₋₃ alkyl;
R₁₂ is H, —C₁₋₃ alkyl, or -methylene-phenylene-NH—C(═O)—NR₁₀R₁₁;
each R₁₃ independently is H, —C₁₋₄ alkyl, —C₁₋₄ hydroxyalkyl, —C₁₋₃ fluoroalkyl, —C₃₋₅ cycloalkyl, or —C₃₋₅ heterocycloalkyl; wherein the —C₁₋₄ alkyl of R₁₃ is optionally substituted with one or more of cyclopropyl or methoxy; wherein the —C₃₋₅ cycloalkyl of R₁₃ is optionally substituted with —C₁₋₃ alkyl;
n is 1 or 2; and
wherein the respective heterocycles each independently include 1 to 3 heteroatoms including at least one of N, O, or S.

In an aspect, R₃ is —C₁₋₃ alkyl or —C₂₋₁₀ heterocycle; the —C₂₋₁₀ heterocycle is —C₂₋₅ heteroaryl; the —C₁₋₃ alkyl of R₃ is optionally substituted with one, two, or three of oxo or —NR₁₂R_{12A}; and the —C₂₋₅ heterocycle of R₃ is optionally substituted with —C₁₋₃ alkyl.

In an aspect, the dashed line refers to the bond being a single bond or a double bond; if the dashed line is a double bond, then R_{4B} is absent and n is 1.

In an aspect, R₁ is —NHR₈; and R₈ is —C₁₋₃ alkyl substituted with one, two, or three of oxo or —NR₉R₁₀.

In an aspect, R₂ is H; and R₃ is —C₁₋₃ alkyl substituted with oxo and —NR₁₂R_{12A}.

In an aspect, at least one of X₅ is CH; X₇ is CH or N; X₈ is CH; R₁ is —NHR₈, —C₂₋₅ heterocycle, or —C₁₋₃ alkyl; R_{4A} is H or —C₁₋₃ alkyl; each R₅ independently is H, halogen, or —C₁₋₃ alkyl; or the two R₅ groups form cyclopropyl; R₆ is H or —C₁₋₃ alkyl; or R₇ is H or —C₁₋₃ alkyl.

In an aspect, R₃ is —C₁₋₃ alkyl substituted with oxo and —NR₁₂R_{12A} and R₁ is —NHR₈ or —C₁₋₃ alkyl substituted with oxo and —NR₁₀R₁₁.

In an aspect, R3 can be —C(═O)—NR₁₂R_{12A} and R1 can be —NH—CO—NR₁₀R₁₁.

In an aspect, X₃ is CR₁₃ and X₄ is CH or N.

In an aspect, R₁ is —NHR₃; wherein R₃ is —C₁₋₃ alkyl or a —C₃₋₄ heterocycle; wherein the —C₁₋₃ alkyl of R₃ is substituted with oxo and —NR₉R₁₀; wherein the —C₃₋₄ heterocycle is substituted with one, two, or three of oxo or —C₁₋₃ alkyl.

In an aspect, X₁ is N, X₃ is N or NR₁₃, and X₄ is N.

In an aspect, the —C₂₋₆ heterocycle of R₃ is a —C₂₋₁₀ heterocycle and R₃ is optionally substituted with one, two, or three of —C₁₋₃ alkyl.

In an aspect, at least one hydrogen (H) is deuterium (D).

In an aspect, R₁ is

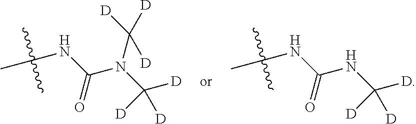

In an aspect, R_{1A} is deuterium; X₅, X₉, and X₁₀ are each CR_{1B}; and the R_{1B} of each of X₅, X₉, and X₁₀ is deuterium.

In an aspect, the compound is a deuterated compound of any of Examples D1, D2, D6, D31, D33, D128, or D132.

In an aspect, a compound or a pharmaceutically acceptable salt thereof wherein the compound is one of: N-(4-(3,3-dimethylureido)benzyl)-1-isopropyl-5-(5-(2-oxopyrrolidin-1-yl)pyridin-2-yl)-4,5,6,7-tetrahydro-1H-pyrazolo[4,3-c]pyridine-3-carboxamide; 5-(5-(Dimethylcarbamoyl)pyridin-2-yl)-N-(4-(3,3-dimethylureido)benzyl)-1-isopropyl-4,5,6,7-tetrahydro-1H-pyrazolo[4,3-c]pyridine-3-carboxamide; 5-(5-(6,7-dihydro-5H-pyrrolo[3,4-b]pyridine-6-carbonyl)pyridin-2-yl)-N-(4-(3,3-dimethylureido)benzyl)-1-isopropyl-4,5,6,7-tetrahydro-1H-pyrazolo[4,3-c]pyridine-3-carboxamide; 5-(5-(Dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide; (S)-5-(5-(dimethylcarbamoyl)pyridin-2-yl)-N-(4-(3,3-dimethylureido)benzyl)-1-isopropyl-7-methyl-4,5,6,7-tetrahydro-1H-pyrazolo[4,3-c]pyridine-3-carboxamide; (S)-5-(4-(dimethylcarbamoyl)phenyl)-1-isopropyl-7-methyl-N-(4-(4-methylpiperazine-1-carboxamido)benzyl)-4,5,6,7-tetrahydro-1H-pyrazolo[4,3-c]pyridine-3-carboxamide; or 5-(5-(dimethylcarbamoyl)pyridin-2-yl)-N-(4-(3,3-dimethylureido)benzyl)-1-(2,2,2-trifluoroethyl)-4,5,6,7-tetrahydro-1H-pyrazolo[4,3-c]pyridine-3-carboxamide.

In an aspect, a compound wherein the compound is one of: N-(4-(3,3-dimethylureido)benzyl)-1-isopropyl-5-(5-(2-oxopyrrolidin-1-yl)pyridin-2-yl)-4,5,6,7-tetrahydro-1H-pyrazolo[4,3-c]pyridine-3-carboxamide; 5-(5-(Dimethylcarbamoyl)pyridin-2-yl)-N-(4-(3,3-dimethylureido)benzyl)-1-isopropyl-4,5,6,7-tetrahydro-1H-pyrazolo[4,3-c]pyridine-3-carboxamide; 5-(5-(6,7-dihydro-5H-pyrrolo[3,4-b]pyridine-6-carbonyl)pyridin-2-yl)-N-(4-(3,3-dimethylureido)benzyl)-1-isopropyl-4,5,6,7-tetrahydro-1H-pyrazolo[4,3-c]pyridine-3-carboxamide; 5-(5-(Dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide; (S)-5-(5-(dimethylcarbamoyl)pyridin-2-yl)-N-(4-(3,3-dimethylureido)benzyl)-1-isopropyl-7-methyl-4,5,6,7-tetrahydro-1H-pyrazolo[4,3-c]pyridine-3-carboxamide; (S)-5-(4-(dimethylcarbamoyl)phenyl)-1-isopropyl-7-methyl-N-(4-(4-methylpiperazine-1-carboxamido)benzyl)-4,5,6,7-tetrahydro-1H-pyrazolo[4,3-c]pyridine-3-carboxamide; or 5-(5-(dimethylcarbamoyl)pyridin-2-yl)-N-(4-(3,3-dimethylureido)benzyl)-1-(2,2,2-trifluoroethyl)-4,5,6,7-tetrahydro-1H-pyrazolo[4,3-c]pyridine-3-carboxamide.

In an aspect, a compound or a pharmaceutically acceptable salt thereof is

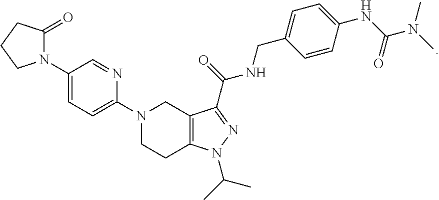 [SMILES: CC(C)n1nc(C(=O)NCc2ccc(NC(=O)N(C)C)cc2)c2c1CCN(c1ccc(N3CCCC3=O)cn1)C2]

In an aspect, a compound or a pharmaceutically acceptable salt thereof is

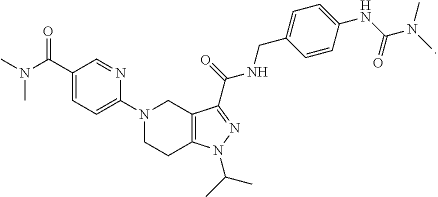

In an aspect, a compound or a pharmaceutically acceptable salt thereof is

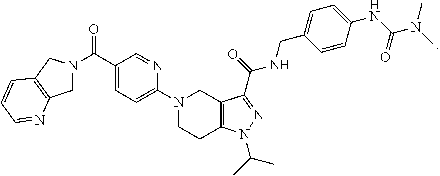 [SMILES: CC(C)n1nc(C(=O)NCc2ccc(NC(=O)N(C)C)cc2)c2c1CCN(c1ccc(C(=O)N3Cc4cccnc4C3)cn1)C2]

In an aspect, a compound or a pharmaceutically acceptable salt thereof is

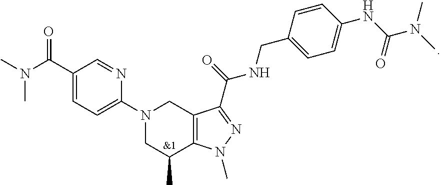

In an aspect, a compound or a pharmaceutically acceptable salt thereof is

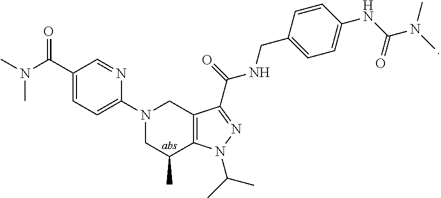

In an aspect, a compound or a pharmaceutically acceptable salt thereof is

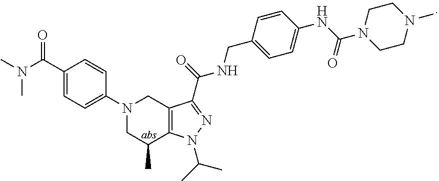

In an aspect, a compound or a pharmaceutically acceptable salt thereof is

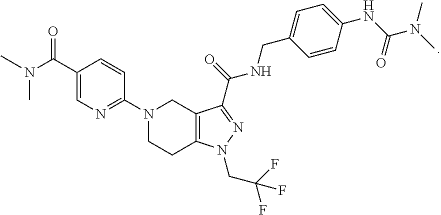

In an aspect, the compound is other than the following: rac-(4R,7S)-9-(5-(dimethylcarbamoyl)pyridin-2-yl)-N-(4-(3,3-dimethylureido)benzyl)-1-methyl-1,4,5,6,7,8-hexahydro-4,7-epiminocyclohepta[c]pyrazole-3-carboxamide, rac-(R)—N-(4-(3,3-dimethylureido)benzyl)-5-(5-(5-(hydroxymethyl)-2-oxooxazolidin-3-yl)pyridin-2-yl)-1-isopropyl-4,5,6,7-tetrahydro-1H-pyrazolo[4,3-c]pyridine-3-carboxamide, N-(4-(3,3-dimethylureido)benzyl)-1-isopropyl-5-(5-((1S,4S)-6-oxo-2,5-diazabicyclo[2.2.1]heptane-2-carbonyl)pyridin-2-yl)-4,5,6,7-tetrahydro-1H-pyrazolo[4,3-c]pyridine-3-carboxamide, rel-(R or S)—N-(4-(3,3-dimethylureido)benzyl)-1-isopropyl-5-(5-(tetrahydro-2H-pyran-2-yl)pyridin-2-yl)-4,5,6,7-tetrahydro-1H-pyrazolo[4,3-c]pyridine-3-carboxamide, rel-(R or S)—N-(4-(3,3-dimethylureido)benzyl)-1-isopropyl-5-(5-(tetrahydro-2H-pyran-2-yl)pyridin-2-yl)-4,5,6,7-tetrahydro-1H-pyrazolo[4,3-c]pyridine-3-carboxamide, or (S)-5-(4-(dimethylcarbamoyl)phenyl)-1-isopropyl-7-methyl-N-(4-(4-methylpyrimidin-2-yl)benzyl)-4,5,6,7-tetrahydro-1H-pyrazolo[4,3-c]pyridine-3-carboxamide.

In an aspect, a pharmaceutical composition comprises the compound of Formula I or a pharmaceutically acceptable salt thereof, and at least one pharmaceutically acceptable excipient.

In an aspect, a method for treating atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa comprises administering to a subject in need thereof a therapeutically effective amount of the compound of Formula I, or a pharmaceutically acceptable salt thereof.

In an aspect, a compound of Formula I, or a pharmaceutically acceptable salt thereof, for use as a medicament.

In an aspect, a compound of Formula I, or a pharmaceutically acceptable salt thereof, can be for use in the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa.

In an aspect, a compound of Formula I, or a pharmaceutically acceptable salt thereof, can be for use in the treatment of at least one of a dermatological condition or a respiratory condition.

In an aspect, use of a compound of Formula I, or a pharmaceutically acceptable salt thereof, can be for the manufacture of a medicament for the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa.

In an aspect, use of a compound, or a pharmaceutically acceptable salt thereof, can be for use in the treatment of at least one of a dermatological condition or a respiratory condition.

In an aspect, the compound or a pharmaceutically acceptable salt thereof is selected from the group consisting of the compounds of Examples 1 and 2 and those listed in Table 2.

In an aspect, a method for treating atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa comprises administering to a subject in need thereof a therapeutically effective amount of the compound of the any of the preceding aspects, or a pharmaceutically acceptable salt thereof. The method can be for treating atopic dermatitis. The method can be for treating

In an aspect, a compound according to any of the preceding aspects, or a pharmaceutically acceptable salt thereof, is for use as a medicament.

In an aspect, a compound according to any of the preceding aspects, or a pharmaceutically acceptable salt thereof, is for use in the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa.

In an aspect, a compound according to any of the preceding aspects, or a pharmaceutically acceptable salt thereof, is for use in the treatment of at least one of a dermatological condition or a respiratory condition.

In an aspect, a use of a compound according to any of the preceding aspects, or a pharmaceutically acceptable salt thereof, for the manufacture of a medicament for the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa.

In an aspect, a use of a compound according to any of the preceding aspects, or a pharmaceutically acceptable salt thereof, is for use in the treatment of at least one of a dermatological condition or a respiratory condition.

Each of the aspects described herein may be combined with any other aspect(s) described herein not inconsistent with the aspect(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the aspect(s) described herein.

Furthermore, each of the aspects described herein envisions within its scope pharmaceutically acceptable salts of the compounds described herein.

Unless otherwise defined herein, scientific and technical terms used in connection with the present disclosure have the meanings that are commonly understood by those of ordinary skill in the art.

The disclosure described herein suitably may be practiced in the absence of any element(s) not specifically disclosed herein.

The compound(s) of the disclosure refers to the compounds of Formula I and Formula IA, where a compound of Formula I can be a compound specifically of Formula IA. One of ordinary skill in the art will appreciate that the compounds of the disclosure include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that the compounds of the disclosure include solvates, hydrates, isomorphs, polymorphs, esters, salt forms, prodrugs, and isotopically labelled versions thereof, where they may be formed. Compounds of the disclosure can include novel intermediates used in the preparation thereof.

As used herein, the singular form “a”, “an”, and “the” include plural references unless indicated otherwise. For example, “a” substituent includes one or more substituents. The term “or” means “and/or” unless clearly indicated otherwise.

As used herein, the term “about” when used to modify a numerically defined parameter means that the parameter may vary by as much as 10% below or above the stated numerical value for that parameter. For example, a dose of about 5 mg means 5%±10%, i.e., it may vary between 4.5 mg and 5.5 mg.

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo, and iodo (F, Cl, Br, I). More specifically, halogen can refer to fluoro and chloro.

“Cyano” refers to a substituent having a carbon atom joined to a nitrogen atom by a triple bond, i.e., —C≡N. “Cyanoimine” refers to a substituent having a carbon atom joined to a nitrogen atom by a double bond and that is bound to a cyano group, i.e., —C(R)═N—C≡N, where R can be a dimethylamino group, i.e., —C(N(CH₃)₂)═N—C≡N.

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁₋₁₂ alkyl”), 1 to 8 carbon atoms (“C₁₋₈ alkyl”), 1 to 6 carbon atoms (“C₁₋₆ alkyl”), 1 to 5 carbon atoms (“C₁₋₅ alkyl”), 1 to 4 carbon atoms (“C₁₋₄ alkyl”), 1 to 3 carbon atoms (“C₁₋₃ alkyl”), or 1 to 2 carbon atoms (“C₁₋₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein. In some instances, substituted alkyl groups are specifically named by reference to the substituent group. For example, “haloalkyl” refers to an alkyl group having the specified number of carbon atoms that is substituted by one or more halo substituents, up to the available valence number.

“Haloalkyl” refers to an alkyl group as defined above containing the specified number of carbon atoms wherein at least one hydrogen atom has been replaced by halogen. Haloalkyl groups man contain, but are not limited to, 1-6 carbon atoms (“C₁₋₆ haloalkyl”), 1-4 carbon atoms (“C₁₋₄ haloalkyl”), or 1-2 carbon atoms (“C₁₋₂ haloalkyl”). More specifically, fluorinated alkyl groups may be specifically referred to as “fluoroalkyl.” Examples of fluoroalkyl groups include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Hydroxyalkyl” refers to an alkyl group as defined above containing the specified number of carbon atoms wherein at least one hydrogen atom has been replaced by hydroxy (OH), for example, only one hydrogen atom is replaced by hydroxy. Hydroxyalkyl groups man contain, but are not limited to, 1-6 carbon atoms (“C₁₋₆ hydroxyalkyl”), 1-4 carbon atoms (“C₁₋₄ hydroxyalkyl”), or 1-2 carbon atoms (“C₁₋₂ hydroxyalkyl”).

“Cyanoalkyl” refers to an alkyl group as defined above containing the specified number of carbon atoms wherein at least one hydrogen atom has been replaced by cyano (—C≡N). Cyanoalkyl groups man contain, but are not limited to, 1-6 carbon atoms (“C₁₋₆ cyanoalkyl”), 1-4 carbon atoms (“C₁₋₄ cyanoalkyl”), or 1-2 carbon atoms (“C₁₋₂ cyanoalkyl”).

“Oxoalkyl” refers to an alkyl group as defined above containing the specified number of carbon atoms wherein at least two hydrogen atoms have been replaced by an oxo group. Oxoalkyl groups man contain, but are not limited to, 1-6 carbon atoms (“C₁₋₆ oxoalkyl”), 1-4 carbon atoms (“C₁₋₄ oxoalkyl”), or 1-2 carbon atoms (“C₁₋₂ oxoalkyl”).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 8 carbon atoms (“C₁₋₃ alkoxy”), 1 to 6 carbon atoms (“C₁₋₆ alkoxy”), 1 to 4 carbon atoms (“C₁₋₄ alkoxy”), or 1 to 3 carbon atoms (“C₁₋₃ alkoxy”). Alkoxy groups include, but are not limited to, methoxy, ethoxy, n-propoxy, isobutoxy, and the like.

“Alkylene” refers to a saturated, divalent aliphatic hydrocarbon radical that has a specified number of carbon atoms, including straight chain or branched chain groups. Alkylene groups may contain, but are not limited to, 1 to 12 carbon atoms (“C₁₋₁₂ alkylene”), 1 to 8 carbon atoms (“C₁₋₈ alkylene”), 1 to 6 carbon atoms (“C₁₋₆ alkylene”), 1 to 5 carbon atoms (“C₁₋₅ alkylene”), 1 to 4 carbon atoms (“C₁₋₄ alkylene”), 1 to 3 carbon atoms (“C₁₋₃ alkylene”), or 1 to 2 carbon atoms (“C₁₋₂ alkylene”).

“Cycloalkyl” refers to a fully or partially saturated hydrocarbon ring system that has the specified number of carbon atoms, which may be a monocyclic, bridged or fused bicyclic, or polycyclic ring system that is connected to the base molecule through a carbon atom of the cycloalkyl ring. “Cycloalkyl” can refer to the ring system being a fully saturated hydrocarbon ring system. Cycloalkyl groups may contain, but are not limited to, 3 to 12 carbon atoms (“C₃₋₁₂ cycloalkyl”), 3 to 8 carbon atoms (“C₃₋₈ cycloalkyl”), 3 to 6 carbon atoms (“C₃₋₆ cycloalkyl”), 3 to 5 carbon atoms (“C₃₋₅ cycloalkyl”) or 3 to 4 carbon atoms (“C₃₋₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein.

“Heterocycloalkyl” refers to a fully or partially saturated ring system containing the specified number of ring atoms and containing at least one heteroatom selected from N, O, and S as a ring member, where ring S atoms are optionally substituted by one or two oxo groups (i.e., S(═O)_{q}, where q is 0, 1 or 2) and where the heterocycloalkyl ring is connected to the base molecule via a ring atom, which may be C or N. The heterocycloalkyl can refer to the ring system being fully saturated. Heterocycloalkyl rings include rings which are spirocyclic, bridged, or fused to one or more other heterocycloalkyl or carbocyclic rings, where such spirocyclic, bridged, or fused rings may themselves be saturated, partially unsaturated, or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(═O)_{q} as ring members, or 1 to 2 ring heteroatoms, provided that such heterocycloalkyl rings do not contain two contiguous oxygen or sulfur atoms.

Heterocycloalkyl rings may be optionally substituted or unsubstituted, as further defined herein. Such substituents may be present on the heterocyclic ring attached to the base molecule, or on a spirocyclic, bridged or fused ring attached thereto. Heterocycloalkyl rings may include, but are not limited to, 3-8 membered heterocyclyl groups, for example 4-7 or 4-6 membered heterocycloalkyl groups, in accordance with the definition herein.

“Aryl” or “aromatic” refers to monocyclic, bicyclic (e.g., biaryl, fused), or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆₋₂₀ aryl”), 6 to 14 carbon atoms (“C₆₋₁₄ aryl”), 6 to 12 carbon atoms (“C₆₋₁₂ aryl”), or 6 to 10 carbon atoms (“C₆₋₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused), or polycyclic ring systems that contain the specified number of ring atoms and include at least one heteroatom selected from N, O, and S as a ring member in a ring in which all carbon atoms in the ring are of sp² hybridization and in which the pi electrons are in conjugation. Heteroaryl groups may contain, but are not limited to, 5 to 20 ring atoms (“5-20 membered heteroaryl”), 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

“Heterocycle” or “heterocyclic” refers to a cyclic ring system that includes at least one heteroatom selected from N, O, and S as a ring member in the ring system. The ring system can include heterocycloalkyl or heteroaryl rings. The ring system can include spirocyclic rings, bridged rings, or fused rings.

“Amino” refers to a group —NH₂, which is unsubstituted or substituted. When substituted, the term includes groups of the form —NRₓR_{y}, one of Rₓ and R_{y} is an alkyl moiety and the other is H, or both of Rₓ and R_{y} are alkyl moieties, where the alkyl moieties have the specified number of carbon atoms (e.g., —NH(C₁₋₃ alkyl) or —N(C₁₋₃ alkyl)₂). The term “aminoheterocycloalkyl” refers to a heterocycloalkyl substituted with an amino group.

The term “pharmaceutically acceptable” means the substance (e.g., the compounds described herein) and any salt thereof, or composition containing the substance or salt of the compounds of the disclosure is suitable for administration to a subject or patient.

A “pharmaceutical composition” refers to a mixture of one or more of the compounds of the disclosure, or a pharmaceutically acceptable salt, solvate, hydrate or prodrug thereof as an active ingredient, and at least one pharmaceutically acceptable excipient.

“Deuterium enrichment factor” as used herein means the ratio between the deuterium abundance and the natural abundance of deuterium, each relative to hydrogen abundance. An atomic position designated as having deuterium typically can have a deuterium enrichment factor of, at least 1,000 (15% deuterium incorporation), at least 2,000 (30% deuterium incorporation), at least 3,000 (45% deuterium incorporation), at least 3,500 (52.5% deuterium incorporation), at least 3,500 (52.5% deuterium incorporation at each designated deuterium atom), at least 4,000 (60% deuterium incorporation), at least 4,500 (67.5% deuterium incorporation), at least 5,000 (75% deuterium incorporation), at least 5,500 (82.5% deuterium incorporation), at least 6,000 (90% deuterium incorporation), at least 6,333.3 (95% deuterium incorporation), at least 6,466.7 (97% deuterium incorporation), at least 6,600 (99% deuterium incorporation), or at least 6,633.3 (99.5% deuterium incorporation).

“Excipient” as used herein describes any ingredient other than the compound(s) of the disclosure. The choice of excipient will to a large extent depend on factors such as the mode of administration, the effect of the excipient on solubility and stability, and the nature of the dosage form. As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof. Examples of excipients also include pharmaceutically acceptable substances such as wetting agents or minor amounts of auxiliary substances such as wetting or emulsifying agents, preservatives, or buffers, which enhance the shelf life or effectiveness of the compound.

The term “treating”, “treat”, or “treatment” as used herein embraces both preventative, i.e., prophylactic, and palliative treatment, i.e., relieve, alleviate, or slow the progression of the patient's disease (or condition) or any tissue damage associated with the disease.

As used herein, the term, “subject”, “individual”, or “patient,” used interchangeably, refers to any animal, including mammals. Mammals according to the disclosure include canine, feline, bovine, caprine, equine, ovine, porcine, rodents, lagomorphs, primates, humans, and the like, and encompass mammals in utero. Humans can be suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition, or disorder in an individual that may be predisposed to the disease, condition, or disorder but does not yet experience or display the pathology or symptomatology of the disease;
(2) inhibiting the disease; for example, inhibiting a disease, condition, or disorder in an individual that is experiencing or displaying the pathology or symptomatology of the disease, condition, or disorder (i.e., arresting (or slowing) further development of the pathology or symptomatology or both); and
(3) ameliorating the disease; for example, ameliorating a disease, condition, or disorder in an individual that is experiencing or displaying the pathology or symptomatology of the disease, condition, or disorder (i.e., reversing the pathology or symptomatology or both).

Salts encompassed within the term “pharmaceutically acceptable salts” refer to the compounds of this disclosure, which are generally prepared by reacting the free base or free acid with a suitable organic or inorganic acid, or a suitable organic or inorganic base, respectively, to provide a salt of the compound of the disclosure that is suitable for administration to a subject or patient.

In addition, the compound of the disclosure may also include other salts of such compounds which are not necessarily pharmaceutically acceptable salts, which may be useful as intermediates for one or more of the following: 1) preparing a compound of the disclosure; 2) purifying the compound of the disclosure; 3) separating enantiomers of the compound of the disclosure; or 4) separating diastereomers of the compound of the disclosure.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts. The salt can be a trifluoroacetate salt or a formate salt.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of the compounds of the disclosure may be prepared by methods well known to one skilled in the art, including but not limited to the following procedures
(i) by reacting a compound of the disclosure with the desired acid or base;
(ii) by removing an acid- or base-labile protecting group from a suitable precursor of a compound of the disclosure or by ring-opening a suitable cyclic precursor, for example, a lactone or lactam, using the desired acid or base; or
(iii) by converting one salt of a compound of the disclosure to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

The compounds of the disclosure, and pharmaceutically acceptable salts thereof, may exist in unsolvated and solvated forms. The term ‘solvate’ is used herein to describe a molecular complex comprising the compound of the disclosure, or a pharmaceutically acceptable salt thereof, and one or more pharmaceutically acceptable solvent molecules, for example, ethanol. The term ‘hydrate’ is employed when said solvent is water.

In addition, the compounds of the disclosure may also include other solvates of such compounds which are not necessarily pharmaceutically acceptable solvates, which may be useful as intermediates for one or more of the following: 1) preparing the compounds of the disclosure; 2) purifying the compounds of the disclosure; 3) separating enantiomers of the compounds of the disclosure; or 4) separating diastereomers of the compounds of the disclosure.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

Also included within the scope of the disclosure are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

The compounds of the disclosure may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the disclosure may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Compounds of the disclosure may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the disclosure containing one or more asymmetric carbon atoms may exist as two or more stereoisomers. Where a compound of the disclosure contains an alkenyl or alkenylene group, geometric cis/trans (or Z/E) isomers are possible. Cis/trans isomers may also exist for saturated rings.

The pharmaceutically acceptable salts of the compounds of the disclosure may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the disclosure contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the disclosure (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

Where structural isomers are interconvertible via a low energy barrier, tautomeric isomerism (‘tautomerism’) may occur. This may take the form of proton tautomerism in the compounds of the disclosure containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the disclosure have been drawn herein in a single tautomeric form, all possible tautomeric forms are included within the scope of the disclosure.

The present disclosure includes all pharmaceutically acceptable isotopically-labeled compounds of Formula I wherein one or more atoms are replaced by atoms having the same atomic number, but an atomic mass or mass number different from the atomic mass or mass number which predominates in nature.

Examples of isotopes suitable for inclusion in the compounds of Formula I may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of Formula I, for example, those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C, ¹⁸F, ¹⁵O and ¹³N, may be useful in Positron Emission Topography (PET) studies for examining substrate receptor occupancy. Substitution with deuterium, i.e., ²H, may afford certain therapeutic advantages resulting from greater metabolic stability, for example, increased in vivo half-life, reduced dosage requirements, reduced CYP450 inhibition (competitive or time dependent), or an improvement in therapeutic index or tolerability.

The disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of Formula I may be defined by the deuterium enrichment factor. It is understood that one or more deuterium may exchange with hydrogen under physiological conditions.

The deuterium compound can be selected from any one of the compounds of the examples specifically illustrated in the Examples section.

One or more hydrogen atoms on certain metabolic sites on the compounds of Formula I can be deuterated.

Isotopically-labeled compounds of Formula I may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the disclosure include those wherein the solvent of crystallization may be isotopically substituted, e.g., D₂O, d₆-acetone, d₆-DMSO.

A compound of the disclosure may be administered in the form of a prodrug. Thus, certain derivatives of a compound of the disclosure which may have little or no pharmacological activity themselves may, when administered into or onto the body, be converted into a compound of the disclosure having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the disclosure may, for example, be produced by replacing appropriate functionalities present in the compounds of the disclosure with certain moieties known to those skilled in the art as ‘pro-moieties’ as described, for example, in ‘Design of Prodrugs’ by H. Bundgaard (Elsevier, 1985).

Thus, a prodrug in accordance with the disclosure may be (a) an ester or amide derivative of a carboxylic acid when present in a compound of the disclosure; (b) an ester, carbonate, carbamate, phosphate or ether derivative of a hydroxyl group when present in a compound of the disclosure; (c) an amide, imine, carbamate or amine derivative of an amino group when present in a compound of the disclosure; (d) a thioester, thiocarbonate, thiocarbamate or sulfide derivatives of a thiol group when present in a compound of the disclosure; or (e) an oxime or imine derivative of a carbonyl group when present in a compound of the disclosure.

Some specific examples of prodrugs in accordance with the disclosure include:
(i) when a compound of the disclosure contains a carboxylic acid functionality (—COOH), an ester thereof, such as a compound wherein the hydrogen of the carboxylic acid functionality of the compound is replaced by C₁₋₈ alkyl (e.g., ethyl) or (—C₁₋₃ alkyl)C(═O)OCH₂— (e.g., ^{t}BuC(═O)OCH₂—);
(ii) when a compound of the disclosure contains an alcohol functionality (—OH), an ester thereof, such as a compound wherein the hydrogen of the alcohol functionality of the compound is replaced by —CO(C₁₋₈ alkyl) (e.g., methylcarbonyl) or the alcohol is esterified with an amino acid;
(iii) when a compound of the disclosure contains an alcohol functionality (—OH), an ether thereof, such as a compound wherein the hydrogen of the alcohol functionality of the compound is replaced by (C₁₋₈ alkyl)C(═O)OCH₂— or —CH₂OP(═O)(OH)₂;
(iv) when a compound of the disclosure contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the disclosure contains a primary or secondary amino functionality (—NH₂ or —NHR where R≠H), an amide thereof, for example, a compound wherein, as the case may be, one or both hydrogens of the amino functionality of the compound is/are replaced by (C₁₋₁₀)alkanoyl, —COCH₂NH₂ or the amino group is derivatized with an amino acid;
(vi) when a compound of the disclosure contains a primary or secondary amino functionality (—NH₂ or —NHR where R #H), an amine thereof, for example, a compound wherein, as the case may be, one or both hydrogens of the amino functionality of the compound is/are replaced by —CH₂OP(═O)(OH)₂.

Certain compounds of the disclosure may themselves act as prodrugs of other compounds the disclosure it is also possible for two compounds of the disclosure to be joined together in the form of a prodrug. In certain circumstances, a prodrug of a compound of the disclosure may be created by internally linking two functional groups in a compound of the disclosure, for instance by forming a lactone.

Also included within the scope of the disclosure are active metabolites of compounds of the disclosure, that is, compounds formed in vivo upon administration of the drug, often by oxidation or dealkylation. Some examples of metabolites in accordance with the disclosure include, but are not limited to,
(i) where the compound of the disclosure contains an alkyl group, a hydroxyalkyl derivative thereof (—CH→—COH):
(ii) where the compound of the disclosure contains an alkoxy group, a hydroxy derivative thereof (—OR→—OH);
(iii) where the compound of the disclosure contains a tertiary amino group, a secondary amino derivative thereof (—NRR′→—NHR or —NHR′);
(iv) where the compound of the disclosure contains a secondary amino group, a primary derivative thereof (—NHR→—NH₂);
(v) where the compound of the disclosure contains a phenyl moiety, a phenol derivative thereof (-Ph→-PhOH);
(vi) where the compound of the disclosure contains an amide group, a carboxylic acid derivative thereof (—CONH₂→COOH); and
(vii) where the compound contains a hydroxy or carboxylic acid group, the compound may be metabolized by conjugation, for example, with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

The compound or a pharmaceutically acceptable salt thereof can have the structure of Formula I or Formula IA or Formula IB. The compound of Formula I can have the structure of Formula IA or Formula IB.

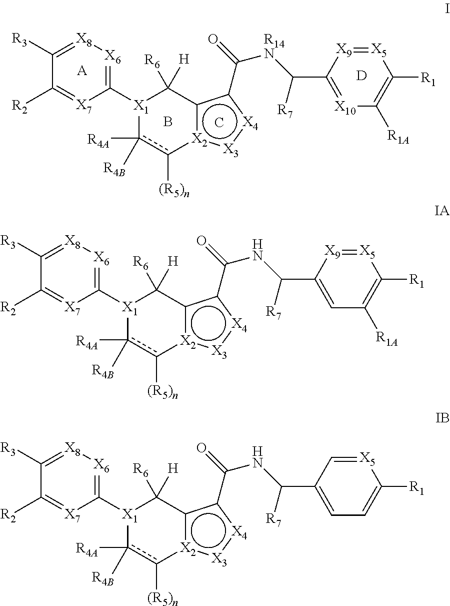

X₁ can be CH, CNH₂, or N. X₁ can be N.
X₂ can be C or N.
X₃ can be CR₁₃, N, NR₁₃, O, or S. X₃ can be CR₁₃. X₃ can be N or NR₁₃. X₃ can be CR₁₃ or N.
X₄ can be CH, N, or NR₁₃. X₄ can be CH, N, or N-cyclopropyl. X₄ can be N.
X₅ can be CH, CR_{1B}, or N. X₅ can be CH.
X₆ can be CH or N.
X₇ can be CH, N, or CF. X₇ can be CH or N. X₇ can be CCl.
X₈ can be CH or N. X₈ can be CH.
X₉ can be CH, CR_{1B}, or N.
X₁₀ can be CH, CR_{1B}, or N. At least one of X₂ can be N or X₃ can be N, NH, or NR₁₃, and X₄ can be N.

If X₃ is S or O, then X₂ can be C.

If X₃ is CR₁₃ or NR₁₃, then X₄ can be CH or N. If X₄ is NR₁₃, then X₃ can be N, O, or S.

The dashed line refers to the bond being a single bond (i.e., the dashed line is not present leaving only the single bond) or a double bond (i.e., the dashed line is present as a second bond making the double bond). The dashed line can refer to the bond being a single bond. If the dashed line refers to the bond being a double bond, then R_{4B} is absent and n is 1.

R₁ can be —NHR₈, —OH, —C₂₋₅ heterocycle, or —C₁₋₃ alkyl. R₁ can be —NHR₈, —C₂₋₅ heterocycle, or —C₁₋₃ alkyl. R₁ can be —C₂₋₅ heterocycle and can optionally be substituted with one or more or one, two, or three of —C₁₋₃ alkyl, —C₁₋₃ oxoalkyl, —C₃₋₅ heterocycloalkyl, —C₃₋₅ hydroxyheterocycloalkyl, —C₃₋₅ aminoheterocycloalkyl, or oxo. R₁ can be —C₂₋₅ heterocycle and can optionally be substituted with one or more or one, two, or three of —C₁₋₃ alkyl or oxo. R₁ can be —C₂₋₅ heterocycle and can optionally be substituted with oxo. R₁ can be —C₁₋₃ alkyl and can be substituted with one, two, or three of oxo, —NR_{10A}R₁₁, or —NR₁₀R₁₁. R₁ can be —C₁₋₃ alkyl and can be substituted with one or two of oxo, —NR_{10A}R₁₁, or —NR₁₀R₁₁. R₁ can be —C₁₋₃ alkyl optionally substituted with one or more or one, two, or three of oxo or —NR₁₀R₁₁. R₁ can be —C₁₋₃ alkyl substituted with oxo and —NR₁₀R₁₁. R₁ can be —C₁₋₃ alkyl substituted with oxo and —NR_{10A}R₁₁. R₁ can be —NHR₈. Each of the —C₂₋₅ heterocycle, —C₃₋₅ heterocycloalkyl, —C₃₋₅ hydroxyheterocycloalkyl, and the —C₃₋₅ aminoheterocycloalkyl independently can include 1 to 4 heteroatoms including at least one of N, O, or S. The —C₂₋₅ heterocycle of R₁ can include 1 to 4 heteroatoms including at least one of N, O, or S; where the heteroatoms can all be N.

R_{1A} can be H. R_{1A} can be —CH₃. R_{1A} can be H, halogen, or —CH₃. R_{1A} can be H or halogen, where the halogen can be F.

R₁ and R_{1A} can form a C₅₋₇ heterocycloalkyl fused to Ring D or a C₅₋₇ heteroaryl fused to Ring D. The C₅₋₇ heterocycloalkyl or the C₅₋₇ heteroaryl can optionally be substituted with oxo. R₁ and R_{1A} can form a C₆₋₈ fused heterocycloalkyl that is optionally substituted with oxo. R₁ and R_{1A} can form a C₆₋₈ fused heteroaryl that is optionally substituted with oxo. Each of the C₆₋₈ fused heterocycloalkyl and the can C₆₋₈ fused heteroaryl independently can include 1 to 3 heteroatoms including at least one of N, O, or S.

Each R_{1B} independently is H, —CH₃, F, Cl, or methoxy.

R₂ can be H, —C₁₋₃ alkyl, —C₁₋₃ alkoxy, —C₁₋₃ fluoroalkyl, or halogen. R₂ can be H.

R₃ can be —C₁₋₃ alkyl, —C₂₋₁₀ heterocycle, —P(═O)(CH₃)₂, —S(═O)CH₃, —NH—S(═O)₂CH₃, or —NH—C(═O)—C₁₋₃ alkyl. R₃ can be —C₁₋₃ alkyl, —C₂₋₁₀ heterocycle, —P(═O)(CH₃)₂, —S(═O)CH₃, or —NH—S(═O)₂CH₃. R₃ can be —C₁₋₃ alkyl, —C₂₋₁₀ heterocycle, or —NH—C(═O)—C₁₋₃ alkyl. R₃ can be —C₁₋₃ alkyl optionally substituted with one or more or one, two, or three of oxo, —OH, halogen, —C₃₋₆ cycloalkyl, —NR₁₂R_{12A}, —C₅₋₈ heterocycle, or cyano. R₃ can be —C₁₋₃ alkyl substituted with oxo and —NR₁₂R_{12A}. The —C₂₋₁₀ heterocycle of R₃ can be —C₂₋₁₀ heterocycloalkyl or —C₂₋₁₀ heteroaryl. The —C₂₋₁₀ heterocycle of R₃ can be —C₂₋₆ heterocycloalkyl or —C₂₋₆ heteroaryl. R₃ can be —C₂₋₁₀ heterocycle optionally substituted with one or more or one, two, or three of —OH, halogen, —C₁₋₃ hydroxyalkyl, —C₁₋₃ alkoxy, —C₁₋₃ alkyl, —C₁₋₃ fluoroalkyl, cyclopropyl, or oxo. R₃ can be —C₂₋₁₀ heterocycloalkyl optionally substituted with one or more or one, two, or three of —OH, halogen, —C₁₋₃ hydroxyalkyl, —C₁₋₃ alkyl, cyclopropyl, or oxo. R₃ can be —C₁₋₃ alkyl optionally substituted with one or more or one, two, or three of oxo or —NR₁₂R_{12A}. R₃ can be —C₂₋₅ heterocycle optionally substituted with —C₁₋₃ alkyl. R₃ can be —C₂₋₅ heteroaryl optionally substituted with one or more or one, two, or three —C₁₋₃ alkyl. Each of the —C₂₋₁₀ heterocycle, —C₅₋₈ heterocycle, —C₂₋₆ heterocycloalkyl, —C₂₋₁₀ heterocycloalkyl, —C₂₋₁₀ heteroaryl, —C₂₋₆ heteroaryl, or —C₂₋₅ heterocycle independently can include 1 to 3 heteroatoms including at least one of N, O, or S. The —C₂₋₁₀ heterocycle of R₃ can include 1 to 3 heteroatoms including at least one of N, O, or S. The —C₂₋₁₀ heterocycle of R₃ can be a spiro, fused, or bridged —C₂₋₁₀ heterocycle. The C₅₋₈ heterocycle of R₃ can include 1 to 4 heteroatoms including at least one of N, O, or S.

R₂ and R₃ can form a C₃₋₆ heterocycle fused to Ring A optionally substituted with one, two, or three of —C₁₋₃ alkyl, —C₂₋₃ oxoalkyl, or oxo. R₂ and R₃ can form a C₃₋₆ heterocycloalkyl fused to Ring A optionally substituted with one or more or one, two, or three of —C₁₋₃ alkyl, —C₂₋₃ oxoalkyl, or oxo. Each of the C₃₋₆ heterocycle or the C₃₋₆ heterocycloalkyl independently can include 1 to 3 heteroatoms including at least one of N, O, or S.

R_{4A} can be H, —OH, —C₁₋₃ fluoroalkyl, or —C₁₋₃ alkyl. R_{4A} can be H, —C₁₋₃ fluoroalkyl, or —C₁₋₃ alkyl.

R_{4B} can be H. R_{4B} can be absent.

R_{4A} and R_{4B} taken together can form cyclopropyl.

R_{4B} and one R₅ taken together can form a C₃₋₅ cycloalkyl fused to Ring B.

Each R₅ independently can be H, —OH, —C₃₋₆ cycloalkyl (e.g., cyclopropyl), halogen, —C₁₋₃ fluoroalkyl, or —C₁₋₃ alkyl. Each R₅ independently can be H, halogen, or —C₁₋₃ alkyl. The two R₅ groups taken together can form cyclopropyl. If the dashed line represents a double bond being present, then R₅ can be H, —C₃₋₆ cycloalkyl (e.g., cyclopropyl), halogen, —C₁₋₃ fluoroalkyl, or —C₁₋₃ alkyl.

R₆ can be H, —OH, or —C₁₋₃ alkyl. R₆ can be H or —C₁₋₃ alkyl.

R_{4A} and R₆ taken together can form a C₁₋₃ alkyl bridge or a C₁₋₃ heteroalkyl bridge. One R₅ and R₆ taken together can form a C₁₋₃ alkyl bridge or a C₁₋₃ heteroalkyl bridge. The C₁₋₃ heteroalkyl bridge can include one heteroatom of N, O, or S.

R₇ can be H, —C₁₋₃ alkyl, or —C₁₋₃ hydroxyalkyl. R₇ can be H or —C₁₋₃ alkyl.

R₃ can be H, —C₁₋₃ alkyl, —SO₂CH₃, or a —C₃₋₄ heterocycle. R₃ can be H, —C₁₋₃ alkyl, or a —C₃₋₄ heterocycle. R₃ can be —C₁₋₃ alkyl substituted with one, two, or three of oxo, —C₃₋₉ heterocycloalkyl, —C₃₋₉ heterocycloalkyl substituted with methyl, —C₁₋₃ alkoxy, cyanoimine, or —NR₉R₁₀. R₃ can be —C₁₋₃ alkyl optionally substituted with one or more or one, two, or three of oxo or —NR₉R₁₀. R₃ can be —C₁₋₃ alkyl optionally substituted with one or two of oxo or —NR₉R₁₀. R₃ can be —C₁₋₃ alkyl substituted with oxo and —NR₉R₁₀. R₃ can be —C₃₋₄ heterocycle optionally substituted with one, two, or three of oxo, halogen, —C₀₋₁ alkylene-NR₁₀R₁₁, —OH, —C₁₋₃ hydroxyalkyl, —C₁₋₃ alkyl-C₁₋₃ alkoxy, —C₁₋₃ oxoalkyl, —C₁₋₃ alkyl, or —C₁₋₃ alkoxy optionally substituted with —NR₁₀R₁₁. The —C₃₋₉ heterocycloalkyl of R₃ can be substituted with one, two, or three of —C₁₋₃ alkyl, —OH, —C₁₋₃ hydroxyalkyl, —O—C₁₋₃ hydroxyalkyl, —C₁₋₃ alkoxy, —C₁₋₃ alkyl-C₁₋₃ alkoxy, halogen, —C₁₋₃ fluoroalkyl, —C₁₋₃ fluoroalkoxy, cyano, —C₁₋₃ cyanoalkyl, —C₀₋₁ alkylene-C₃₋₅ heterocycloalkyl, —O—C₃₋₅ heterocycloalkyl, —C₀₋₁ alkylene-NR₁₀R₁₁. R₈ can be —C₃₋₄ heterocycle optionally substituted with one or more or one, two, or three of oxo or —C₁₋₃ alkyl. Each of the —C₃₋₄ heterocycle or the —C₃₋₉ heterocycloalkyl of R₃ independently can include 1 to 4 heteroatoms including at least one of N, O, or S. The —C₃₋₄ heterocycle of R₃ can include 1 to 4 heteroatoms including at least one of N or O. The —C₃₋₉ heterocycloalkyl of R₃ can include 1 to 3 heteroatoms including at least one of N or O. The —C₃₋₄ heterocycle or the —C₃₋₉ heterocycloalkyl of R₃ can be a bridged, spiro, or fused heterocycle.

R₉ can be H, —C₁₋₄ alkyl, —C₁₋₄ hydroxyalkyl, —C₁₋₃ fluoroalkyl, —C₃₋₅ cycloalkyl, -methylene-phenylene-NH—C(═O)—NR₁₀R₁₁, or —C₃₋₅ heterocycloalkyl. R₉ can be the —C₁₋₄ alkyl optionally substituted with one or more or one, two, or three of —C₃₋₅ cycloalkyl, —C₃₋₅ heterocycloalkyl, or methoxy. R₉ can be —C₃₋₅ cycloalkyl of R₉ optionally substituted with one or two of —OH or —C₁₋₃ alkyl. R₉ can be H or —C₁₋₄ alkyl. Each of the —C₃₋₅ heterocycloalkyl of R₉ independently can include 1 to 2 heteroatoms including at least one of N or O.

Each R₁₀ independently can be H or —C₁₋₄ alkyl. Each R₁₀ independently can be H or —C₁₋₃ alkyl.

R_{10A} can be —C₀₋₁ alkylene-C₃₋₅ heterocycloalkyl optionally substituted with —C₁₋₃ alkyl.

The C₃₋₅ heterocycloalkyl of R_{10A} can include 1 heteroatom of N.

If the —C₁₋₃ alkyl of R₃ is substituted with —NR₉R₁₀, then the corresponding R₉ and R₁₀ can optionally be combined to form the C₃₋₉ heterocycloalkyl or the C₃₋₉ heterocycloalkyl.

Each R₁₁ independently can be H or —C₁₋₃ alkyl.

R₁₂ can be H, —C₁₋₃ alkyl, —C₁₋₃ hydroxyalkyl, —(CH₂)₀₋₂—C₃₋₅ heterocycloalkyl, —(CH₂)₁₋₂—O—C₃₋₅ heterocycloalkyl, -methylene-C(═O)—NR₁₀R₁₁, or -methylene-phenylene-NH—C(═O)—NR₁₀R₁₁.

The —(CH₂)₀₋₂—C₃₋₅ heterocycloalkyl or the —(CH₂)₁₋₂—O—C₃₋₅ heterocycloalkyl of R₁₂ can be substituted with —C₁₋₄ alkyl, —C₁₋₃ alkyl-C₁₋₃ alkoxy, —(CH₂)₀₋₁-phenyl, halogen, —C₁₋₃ fluoroalkyl, or —(CH₂)₀₋₁—C₃₋₅ heterocycloalkyl. The —C₁₋₃ alkyl of R₁₂ can be substituted with one, two, or three of oxo or NR₁₀R₁₁. Each of the —(CH₂)₀₋₂—C₃₋₅ heterocycloalkyl, the —(CH₂)₁₋₂—O—C₃₋₅ heterocycloalkyl, or the —(CH₂)₀₋₁—C₃₋₅ heterocycloalkyl of R₁₂ independently can include 1 to 2 heteroatoms including at least one of N or O.

R_{12A} can be H. R_{12A} can be —C₁₋₄ alkyl.

R₁₂ and R_{12A} can form a C₃₋₁₀ heterocycle optionally substituted with one, two, or three of —C₁₋₃ alkyl, —OH, oxo, halogen, —C₂₋₃ oxoalkyl, —C₁₋₃ alkoxy, —C₁₋₃ hydroxyalkyl, —C₁₋₃ alkyl-C₁₋₃ alkoxy, cyano, —C₃₋₆ cycloalkyl, —S(═O)₂CH₃, —S(═O)₂CH₂CH₃, —C(═O)—NR₁₀R₁₁, or —NHC(═O)CH₃. The C₃₋₁₀ heterocycle can include 1 to 4 heteroatoms including at least one of N, O, or S. The C₃₋₁₀ heterocycle can be a bridged, spiro, or fused heterocycle.

R₁₃ can be H, —C₁₋₆ alkyl, —C₁₋₄ hydroxyalkyl, —C₁₋₃ fluoroalkyl, —C₁₋₃ alkyl-C₁₋₃ alkoxy, —C₁₋₄ cyanoalkyl, —C₀₋₁ alkylene-C₃₋₅ cycloalkyl, —C₂₋₄ oxoalkyl, or —C₀₋₁ alkylene-C₃₋₅ heterocycloalkyl. R₁₃ can be H, —C₁₋₄ alkyl, —C₁₋₄ hydroxyalkyl, —C₁₋₃ fluoroalkyl, —C₃₋₅ cycloalkyl, or —C₃₋₅ heterocycloalkyl. R₁₃ can be H. R₁₃ can be —C₀₋₁-cyclopropyl. The —C₁₋₆ alkyl of R₁₃ can optionally be substituted with one, two, or three of oxo, fluoro, —NH₂, C₃₋₆ cycloalkyl, or methoxy. The —C₃₋₅ cycloalkyl or the —C₃₋₆ cycloalkyl of R₁₃ can optionally be substituted with one, two, or three of —C₁₋₃ alkyl, —C₁₋₃ fluoroalkyl, or halogen. The —C₃₋₅ heterocycloalkyl of R₁₃ can include 1 to 3 heteroatoms including at least one of N, O, or S.

R₁₄ can be H.

X₄ can be NR₁₃ and R₁₃ and R₁₄ can form a C₄₋₅ heterocycle fused to Ring C.

The variable n can be 1 or 2.

The variable m can be 0 or 1.

The respective heterocycles can each independently include 1 to 4 heteroatoms including at least one of N, O, or S.

At least one hydrogen of the compound of Formula I can be deuterium.

The A, B, C, and D values used to denote the rings in Formula I can be used to identify the corresponding rings of Formula IA and Formula IB.

The term “one or more of” when defining the number of groups in Formula I, IA, or IB can, for example, refer to one to four, or one to three of the listed options each time independently that the term is used.

The compound or a pharmaceutically acceptable salt thereof can be N-(4-(3,3-dimethylureido)benzyl)-1-isopropyl-5-(5-(2-oxopyrrolidin-1-yl)pyridin-2-yl)-4,5,6,7-tetrahydro-1H-pyrazolo[4,3-c]pyridine-3-carboxamide.

The compound or a pharmaceutically acceptable salt thereof can be 5-(5-(Dimethylcarbamoyl)pyridin-2-yl)-N-(4-(3,3-dimethylureido)benzyl)-1-isopropyl-4,5,6,7-tetrahydro-1H-pyrazolo[4,3-c]pyridine-3-carboxamide.

The compound or a pharmaceutically acceptable salt thereof can be 5-(5-(6,7-dihydro-5H-pyrrolo[3,4-b]pyridine-6-carbonyl)pyridin-2-yl)-N-(4-(3,3-dimethylureido)benzyl)-1-isopropyl-4,5,6,7-tetrahydro-1H-pyrazolo[4,3-c]pyridine-3-carboxamide.

The compound or a pharmaceutically acceptable salt thereof can be 5-(5-(Dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide.

The compound or a pharmaceutically acceptable salt thereof can be (S)-5-(5-(dimethylcarbamoyl)pyridin-2-yl)-N-(4-(3,3-dimethylureido)benzyl)-1-isopropyl-7-methyl-4,5,6,7-tetrahydro-1H-pyrazolo[4,3-c]pyridine-3-carboxamide.

The compound or a pharmaceutically acceptable salt thereof can be (S)-5-(4-(dimethylcarbamoyl)phenyl)-1-isopropyl-7-methyl-N-(4-(4-methylpiperazine-1-carboxamido)benzyl)-4,5,6,7-tetrahydro-1H-pyrazolo[4,3-c]pyridine-3-carboxamide.

The compound or a pharmaceutically acceptable salt thereof can be 5-(5-(dimethylcarbamoyl)pyridin-2-yl)-N-(4-(3,3-dimethylureido)benzyl)-1-(2,2,2-trifluoroethyl)-4,5,6,7-tetrahydro-1H-pyrazolo[4,3-c]pyridine-3-carboxamide.

A pharmaceutical composition can comprise a compound of Formula I. For example, the pharmaceutical composition can comprise a compound of Formula IA.

The compositions of the disclosure may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). The compound can be administered by intravenous infusion or injection. The compound can be administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the disclosure. The oral administration may be in a powder or granule form. The oral dosage form can be sub-lingual, for example, a lozenge. In such solid dosage forms, the compounds of the disclosure are ordinarily combined with one or more adjuvants. Such capsules or tablets may comprise a controlled release formulation. In the case of capsules, tablets, and pills, the dosage forms also may comprise buffering agents or may be prepared with enteric coatings.

Oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

The disclosure comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

The disclosure comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of the disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of the disclosure is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The disclosure comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the disclosure may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250, or 500 milligrams (mg) of the active ingredient for the symptomatic adjustment of the dosage to the patient. A medicament typically contains 0.01 mg to 500 mg of the active ingredient, or 1 mg to 100 mg of active ingredient. Intravenous doses may be in the range of 0.01 to 10 mg/kg/minute during a constant rate infusion.

Liposome containing compounds of the disclosure may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the disclosure may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the disclosure, which matrices are in the form of shaped articles, e.g., films, or microcapsules. Examples of sustained-release matrices include polyesters, hydrogels (for example, poly(2-hydroxyethyl-methacrylate) or poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the disclosure are generally placed into a container having a sterile access port, for example, an intravenous solution bag or vial having a stopper pierceable by a hypodermic injection needle.

Suitable emulsions may be prepared using commercially available fat emulsions, such as a lipid emulsion comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil.

The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets having a diameter of 0.1 to 1.0 micrometers (μm), particularly 0.1 to 0.5 μm, and have a pH of 5.5 to 8.0.

For example, the emulsion compositions may be those prepared by mixing a compound of the disclosure with a lipid emulsion comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. The compositions can be administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent, or intermittent positive pressure breathing machine. Solution, suspension or powder compositions may be administered, preferably, orally or nasally, from devices which deliver the formulation in an appropriate manner.

A drug product intermediate (DPI) is a partly processed material that must undergo further processing steps before it becomes bulk drug product. Compounds of the disclosure may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. The drug product intermediate can contain a compound of the disclosure isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. Amorphous solid dispersions can comprise a compound of the disclosure and a polymer excipient. Other excipients as well as concentrations of said excipients and the compound of the disclosure are well known in the art and are described in standard textbooks. See, for example, “Amorphous Solid Dispersions Theory and Practice” by Navnit Shah et al.

This disclosure further comprises use of a compound of the disclosure for use as a medicament (such as a unit dosage tablet or unit dosage capsule). This disclosure comprises the use of a compound of the disclosure for the manufacture of a medicament (such as a unit dosage tablet or unit dosage capsule) to treat one or more of the conditions previously identified in the above sections discussing methods of treatment. This disclosure further comprises a compound of the disclosure, or a pharmaceutically acceptable salt thereof, for use as a medicament and a compound the disclosure, or a pharmaceutically acceptable salt therefore, for use in any method of treatment herein disclosed.

Typically, a compound of the disclosure is administered in an amount effective to treat a condition as described herein. The compounds of the disclosure may be administered as compound per se, or alternatively, as a pharmaceutically acceptable salt. For administration and dosing purposes, the compound per se or pharmaceutically acceptable salt thereof will simply be referred to as the compounds of the disclosure.

The compounds of the disclosure are administered by any suitable route in the form of a pharmaceutical composition adapted to such a route, and in a dose effective for the treatment intended. The compounds of the disclosure may be administered orally, rectally, vaginally, parenterally, topically, intranasally, or by inhalation.

The compounds of the disclosure may be administered orally. Oral administration may involve swallowing, so that the compound enters the gastrointestinal tract, or buccal or sublingual administration may be employed by which the compound enters the bloodstream directly from the mouth.

The compounds of the disclosure may also be administered parenterally, for example, directly into the bloodstream, into muscle, or into an internal organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle (including microneedle) injectors, needle-free injectors, and infusion techniques.

The compounds of the disclosure may be administered topically to the skin or mucosa, that is, dermally or transdermally. The compounds of the disclosure may also be administered intranasally or by inhalation. The compounds of the disclosure may be administered rectally or vaginally. The compounds of the disclosure may also be administered directly to the eye or ear.

The dosage regimen for the compounds of the disclosure or compositions containing said compounds is based on a variety of factors, including the type, age, weight, sex and medical condition of the patient; the severity of the condition; the route of administration; and the activity of the particular compound employed. Thus, the dosage regimen may vary widely. The total daily dose of a compound of the disclosure can be 0.01 to 100 mg/kg (i.e., mg compound of the disclosure per kg body weight) for the treatment of the indicated conditions discussed herein. The total daily dose of the compound of the disclosure can be 0.1 to 50 mg/kg, or 0.5 to 30 mg/kg. It is not uncommon that the administration of the compounds of the disclosure will be repeated a plurality of times in a day (typically no greater than 4 times). Multiple doses per day typically may be used to increase the total daily dose, if desired.

The compounds of the disclosure may inhibit the activity of STAT6 and may be useful in the treatment, prevention, suppression, and amelioration of disease(s) or diseases, disorders, and conditions mediated by STAT6.

The compounds of the disclosure may be used to treat or prevent at least one disease or condition relating to an inflammatory disorder. The compounds of the disclosure may be used to treat or prevent at least one disease or condition including atopic dermatitis, eosinophilic gastritis, atopic keratoconjunctivitis, allergy (including food allergy, latex allergy, pet allergy, mold allergy, insect allergy, pollen allergy, dust allergy, seasonal allergy, ragweed allergy, drug allergy, allergic rhinitis, allergic rhinitis, allergic fungal rhinosinusitis, allergic contact dermatitis, and allergic bronchopulmonary aspergillosis), alopecia (including alopecia areata), Alzheimer's disease, arteritis, asthma, atherosclerosis, autoimmune disorders (including lupus nephritis, autoimmune hepatitis, myasthenia gravis, Guillain-Barre syndrome, and Graves' disease) Bechet's disease, bullous pemphigoid, cancer, chronic obstructive pulmonary disease, chronic pruritis, chronic urticaria, celiac disease, Crohn's disease (CD), dermatitis (including atopic dermatitis (AD), hand dermatitis, and hand or foot atopic dermatitis), diabetic kidney disease, diversion colitis, eosinophilic esophagitis (including pediatric eosinophilic esophagitis), eye disorders or conditions (including autoimmune diseases of the eye, keratoconjunctivitis, vernal conjunctivitis, non-infectious uveitis (e.g. uveitis associated with Bechet's disease and lens-induced uveitis), keratitis (e.g. herpetic keratitis and conical keratitis), keratoleukoma, ocular premphigus, Mooren's ulcer, scleritis, retinitis, retinopathy, Grave's ophthalmopathy, Vogt-Koyanagi-Harada syndrome, keratoconjunctivitis sicca (dry eye), phlyctenule, iridocyclitis, endocrine ophthalmopathy, sympathetic ophthalmitis, allergic conjunctivitis, and ocular neovascularization), fungal keratitis, gout, idiopathic pulmonary fibrosis (IPF), indeterminate colitis, keloids, mastocytosis, microscopic colitis (such as collagenous colitis or lymphocytic colitis), myositis, nephritis, non-alcoholic steatohepatitis (NASH), pancreatitis, primary biliary cirrhosis, proctitis, prurigo nodularis, psoriasis, psoriatic arthritis, primary biliary cirrhosis, rhinosinusitis (including chronic rhinosinusitis with or without nasal polyps), sarcoidosis, scleroderma, sclerosing cholangitis, Sjogren's syndrome, systemic lupus erythematosus (SLE), systemic sclerosis, thyroiditis, ulcerative colitis (UC), vitiligo, vasculitis, Vogt-Koyanagi-Harada syndrome, Wegener's granulomatosis, or hidradenitis suppurativa. A method of treating or preventing one or one or more symptoms associated with the respective disease or condition comprises administering a pharmaceutical composition comprising a therapeutically effective amount of a compound of the disclosure to a patient.

The compounds of the disclosure may be used to treat or prevent at least one disease or condition including atopic dermatitis, asthma, chronic obstructive pulmonary disease (COPD), rhinosinusitis (for example, chronic rhinosinusitis with nasal polyps or chronic rhinosinusitis without nasal polyps), chronic urticaria (for example, chronic inducible urticaria or chronic spontaneous urticaria), prurigo nodularis, eosinophilic esophagitis, eosinophilic gastritis, bullous pemphigoid, pruritis (for example, chronic pruritis of unknown origin), atopic keratoconjunctivitis, chronic inducible urticaria, or allergy (for example, food allergy).

The compounds of the disclosure may be used to treat or prevent dermatological conditions, such as eczema (e.g. chronic and dyshidrotic eczema), chronic itch, dermatitis (e.g. atopic, irritant contact, allergic contact, occupational, perioral, stasis, nummular, seborrheic, xerotic, eyelid, diaper, and hand dermatitis), vitiligo, alopecia, alopecia areata, pruritis (e.g. chronic idiopathic pruritus), prurigo nodularis, psoriasis (e.g. plaque, guttate, inverse, pustular, nail, flexural palmoplantar, facial or erythrodermic psoriasis), scleroderma, pemphigus, dermatomyositis, neurodermatitis, skin flushing, cutaneous lupus erythematosus (e.g. acute cutaneous lupus (acute skin lupus), subacute cutaneous lupus (subacute lupus), and chronic cutaneous lupus (discoid lupus)), keloid, sunburn, hypertrophic scar, idiopathic thrombocytopenic purpura (ITP), ichthyosis (e.g. ichthyosis vulgaris), epidermal hyperplasia, acne, lichen planus, lichen sclerosis, rosacea, epidermolysis bullosa, intertrigo, keratosis pilaris, urticaria (e.g. chronic spontaneous urticaria, chronic idiopathic urticaria, chronic physical urticaria), molluscum contagiosum, Netherton syndrome, Sweet's syndrome, pityriasis alba, vulvovaginitis, Sutton's nevus/nevi, post inflammatory hypopigmentation, senile leukoderma, chemical/drug-induced leukoderma, palmoplantar pustulosis, pemphigoid, pemphigoid nodularis, bullous pemphigoid, and hidradenitis suppurativa.

The compounds of the disclosure may be used to treat or prevent respiratory conditions, such as rhinitis (e.g. allergic and perennial rhinitis), rhinorrhea, nasal congestion, nasal inflammation, asthma (e.g. chronic asthma, inveterate asthma, late asthma, bronchial asthma, allergic asthma, intrinsic asthma, extrinsic asthma, and dust asthma), chronic obstructive pulmonary disease (COPD), idiopathic pulmonary fibrosis (IPF), chronic and acute bronchoconstriction, chronic bronchitis, emphysema, allergic bronchopulmonary aspergillosis, chronic eosinophilic pneumonia, acute lung injury (ACI), adult respiratory distress syndrome (ARDS), pulmonary vascular disease (PVD), pulmonary arterial hypertension (PAH), bronchiectasis, sinusitis, rhinosinusitis, allergic fungal rhinosinusitis, chronic rhinosinusitis with nasal polyps, pulmonary sarcoidosis, and silicosis.

The compounds of the disclosure may be used to treat or prevent joint disorders, such as arthritis (e.g. osteoarthritis, as well as psoriatic, rheumatoid, juvenile, and gouty arthritis), spondyloarthropathy (e.g. reactive arthritis (also known as Reiter's Syndrome) and axial spondyloarthritis (including ankylosing spondylitis)), cartilage inflammation, bone degradation, and Still's disease; cardiovascular and metabolic disorders, such as diabetes (type 1 and type 2), myocarditis, diabetic neuropathy, atherosclerosis, cachexia, and celiac sprue; neuroinflammatory disorders, such as lupus (e.g. CNS, systemic and discoid lupus), systemic lupus erythematosus (SLE), diabetic neuropathy, autoimmune encephalitis, Alzheimer's disease, Parkinson's disease, and multiple sclerosis; and cancer.

The compound of the disclosure may be used alone, or in combination with one or more other therapeutic agents. The disclosure provides any of the uses, methods or compositions as defined herein wherein the compound of the disclosure, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compound at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the disclosure and the one or more other therapeutic agents may be administered as a fixed or non-fixed combination of the active ingredients. The term “fixed combination” means a compound of the disclosure, or a pharmaceutically acceptable salt thereof, and the one or more therapeutic agents, are both administered to a subject simultaneously in a single composition or dosage. The term “non-fixed combination” means that a compound of the disclosure, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

The compounds of the disclosure can be administered in combination with one or more pharmaceutically active agents other than the compounds of the disclosure including the pharmaceutically acceptable salts of the specifically named agents and the pharmaceutically acceptable solvates of said agents and salts.

The present disclosure can provide a pharmaceutical composition comprising the compound of the disclosure or a pharmaceutically acceptable salt thereof, wherein the pharmaceutical composition is administered in combination with a pharmaceutical composition comprising a different pharmaceutically active compound or a pharmaceutically acceptable salt thereof simultaneously or at different times.

These agents and compounds may be combined with pharmaceutically acceptable vehicles such as saline, Ringer's solution, dextrose solution, and the like. The particular dosage regimen, i.e., dose, timing and repetition, will depend on the particular individual and that individual's medical history.

Another aspect of the disclosure provides kits comprising the compound of the disclosure or pharmaceutical compositions comprising the compound. A kit may include, in addition to the compound of the disclosure or pharmaceutical composition thereof, diagnostic or therapeutic agents. A kit may also include instructions for use in a diagnostic or therapeutic method. The kit can include the compound or a pharmaceutical composition thereof and a diagnostic agent. The kit can include the compound or a pharmaceutical composition thereof and one or more therapeutic agents.

The kits can be suitable for use in performing the methods of treatment described herein. The kit can contain a first dosage form comprising one or more of the compounds of the disclosure in quantities sufficient to carry out the methods of the disclosure. The kit can comprise one or more compounds of the disclosure in quantities sufficient to carry out the methods of the disclosure and a container for the dosage and a container for the dosage.

Compounds of the present disclosure may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of Formula I. It will be further appreciated that it may be necessary or desirable to carry out the transformations in a different order from that described in the schemes, or to modify one or more of the transformations, to provide the desired compound of Formula I.

In the preparation of compounds of Formula I it is noted that some of the preparation methods useful for the preparation of the compounds described herein may require protection of remote functionality (e.g., a primary amine, secondary amine, carboxyl, etc. in a precursor of a compound of Formula I). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains amine or carboxylic acid functionality, such functionality may interfere with reactions at other sites of the molecule if left unprotected.

Accordingly, such functionalities may be protected by an appropriate protecting group (PG) that may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) that are generally not chemically reactive under the reaction conditions described and may typically be removed without chemically altering other functionality in a compound of Formula I.

One skilled in the art will recognize that, in some cases, the compounds will be generated as a mixture of diastereomers and/or enantiomers; these may be separated at various stages of the synthetic scheme using conventional techniques or a combination of such techniques, such as, but not limited to, crystallization, normal-phase chromatography, reversed-phase chromatography and chiral chromatography, to afford the single enantiomers of the disclosure; for example, see “Stereochemistry of Organic Compounds” by E. L. Eliel and S. H. Wilen (Wiley, New York, 1994).

Unless otherwise indicated, the substituents in the schemes are defined as above. Isolation and purification of the products are accomplished by standard procedures, which are known to a chemist of ordinary skill.

It will be understood by one skilled in the art that the various symbols, superscripts, and subscripts used in the schemes, methods, and examples are used for the convenience of representation and/or to reflect the order in which they are introduced in the schemes and are not intended to necessarily correspond to the symbols, superscripts or subscripts in the appended claims. The schemes are representative of methods useful in synthesizing the compounds of the present disclosure. They are not to constrain the scope of the disclosure in any way.

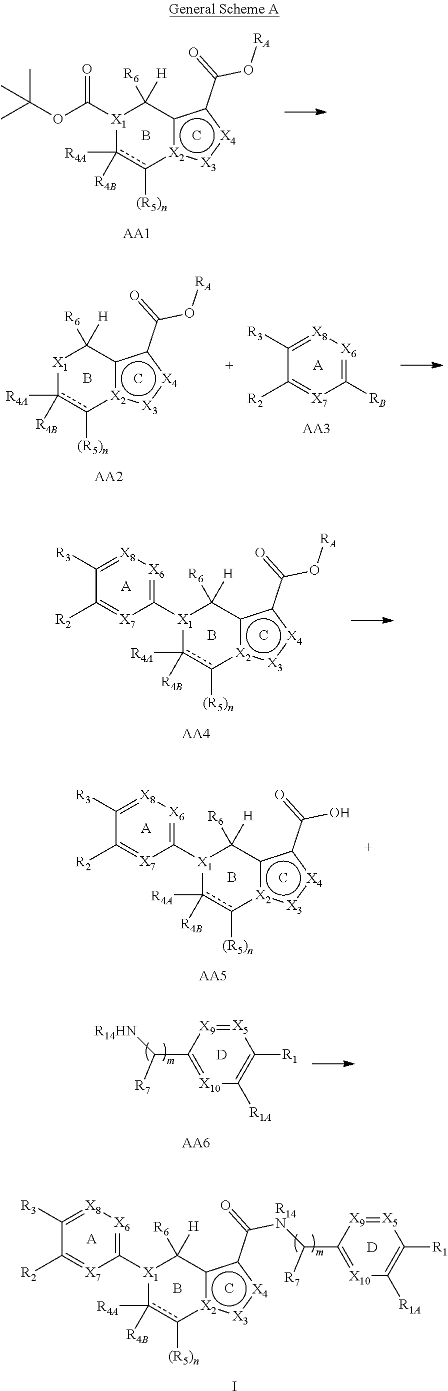

The general structure of Formula I can be prepared as shown in General Scheme A. The tert-butyloxycarbonyl (BOC) protected amine of Formula AA1, synthesized by literature methods or purchased commercially, can be treated with standard acidic deprotection reagents such as hydrochloric acid (HCl) in solvents like 1,4-dioxane (dioxane), ethyl acetate (EtOAc) and dichloromethane (DCM) from 10 degrees Celsius (° C.) to room temperature to afford the deprotected amine compound of Formula AA2. The amine of Formula AA2 can be treated with a halogen (e.g., R_{B}═F or Br)-substituted aromatic of Formula AA3 using standard S_{N}Ar conditions such as N,N-diisopropylethylamine (DIEA), tripotassium phosphate (K₃PO₄), potassium carbonate (K₂CO₃), or cesium fluoride (CsF) in 1-pentanol, butyronitrile, dimethylformamide (DMF), dimethyl sulfoxide (DMSO), or acetonitrile (ACN) when heated between 80 to 140° C. to afford Formula AA4. The Formula AA4 can also be prepared by reacting Formula AA2 and AA3 with copper(I) iodide (CuI), 1,2-dimethylethylenediamine (DMEDA), or trans-N,N′-dimethylcyclohexane-1,2-diamine or Pd catalyst such as (2-dicyclohexylphosphino-2′,6′-diisopropoxy-1,1′-biphenyl)[2-(2′-amino-1,1′-biphenyl)]palladium(II) methanesulfonate (RuPhos Pd G3) with 2-dicyclohexylphosphino-2′,6′-diisopropoxybiphenyl (RuPhos), mesylate[(di(1-adamantyl)-n-butylphosphine)-2-(2′-amino-1,1′-biphenyl)]palladium(II) (cataCXium® A Pd G3), [dicyclohexyl(2′,6′-diisopropoxy-2-biphenylyl)phosphine-κP](methanesulfonatato-κO)[2′-(methylamino-κN)-2-biphenylyl-κC²]palladium (RuPhos Pd G4) with a base such as sodium tert-butoxide (NaOtBu), K₂CO₃, tripotassium phosphate (K₃PO₄) or cesium carbonate (Cs₂CO₃) in a solvent like dioxane, dimethylacetamide (DMA), DMF, DMSO, 2-methyl-2-butanol (tert-amyl alcohol) or toluene and heated between 80 to 130° C.

Formula AA4, where R₃ is a halogen (e.g., F, Br, or I), can be converted to an amine, urea, or lactam using standard SNAr or cross coupling conditions described above to afford Formula AA4. When R₃ is a halogen (e.g. I) on Formula AA4, the halogen can react with a BOC-protected hydrazine with the cross coupling conditions described above then can be deprotected under standard acidic conditions such as HCl in a solvent like dioxane or DCM and cyclized by reacting with (E)-4-(dimethylamino)but-3-en-2-one in EtOH to converted R₃ to a 5-methyl-1H-pyrazole on Formula AA4. For Formula AA4, where R₃ is a halogen (e.g., Br, I), can be converted to a carbon-carbon bond by reacting the halogen with nickel(II) chloride ethylene glycol dimethyl ether complex (NiCl₂·glyme), 5-methoxypicolinimidamide, and using activated ester of N-hydroxyphthalimide, tetrabutylammonium iodide, preactivated zinc (Zn) and trifluoroacetic acid (TFA) in a solvent like DMA from room temperature to 70° C. or under photoredox conditions such as [4,4′-bis(1,1-dimethylethyl)-2,2′-bipyridine-N1,N1′]bis[2-(2-pyridinyl-N)phenyl-C]iridium(III) hexafluorophosphate (Ir(ppy)₂(dtbbpy)PF₆), nickel(II) bromide ethylene glycol dimethyl ether complex (NiBr₂·DME), 5-methoxypicolinimidamide, phthalimide and quinuclidine in a solvent such as DMA and methyl tertiary-butyl ether (MTBE) to afford Formula AA4. When R₃ is a halogen (e.g. Br) on Formula AA4, the halogen can react with B₂Pin₂ and Pd(dppf)Cl₂ with a base like KOAc in a solvent like dioxane at 85° C. to form boronic acid at R₃. The boronic acid at R₃ can react further by reacting with bromo-substituted 5-membered ring heterocycle, Pd(dppf)Cl₂ and with a base like K₃PO₄ in solvents like dioxane at 85° C. to give a 5-membered ring heterocycle at R₃ on Formula AA4. When R₃ is a carboxylic acid on Formula AA4, the acid can be transformed further via amide coupling with HATU and a base like DIEA in a solvent like DMF at 40° C. to form the amide on R₃. When R₃ is a tert-butyl acetate on Formula AA4, it can be deprotected under standard acidic conditions such as TFA or HCl in a solvent like dioxane, H2O, and DCM from room temperature to 40° C., then transformed further under standard amide coupling conditions to form an amide at R₃. When R₃ is a methyl propionate on Formula AA4 then Formula AA4 can react with 2-bromoacetonitrile with a base like lithium bis(trimethylsilyl)amide (LiHMDS) in a solvent like THF at −78° C. which can be transformed further by reacting with sodium borohydride (NaBH₄) in solvents like THF and H₂O at 0° C. to room temperature to form a 5-membered lactam at R₃ on Formula AA4.

The ester (e.g., R_{A}=methyl or ethyl) of Formula AA4 can undergo standard hydrolysis conditions such as utilizing a base (e.g., lithium hydroxide monohydrate (LiOH), sodium hydroxide (NaOH), 1,5,7-triazabicyclo[4.4.0]dec-5-ene, or potassium trimethylsilanolate (KOTMS) in solvents like tetrahydrofuran (THF), methanol (MeOH), ethanol (EtOH), water (H₂O), dioxane, or ACN. The reaction can typically occur at 0 to 50° C., preferably at about 23° C. (room temperature), to afford the carboxylic acid of Formula AA5. The compound of Formula I can be obtained by reacting the carboxylic acid of AA5 with the amine of Formula AA6 under standard amide coupling reagents like 1-(3-dimethylaminopropyl)-3-ethylcarbodiimide hydrochloride (EDCl) with 2-hydroxypyridine-N-oxide (HOPO), 1-[bis(dimethylamino)methylene]-1H-1,2,3-triazolo[4,5-b]pyridinium 3-oxid hexafluorophosphate (HATU), or other common reagents in solvents such as ACN and H₂O, DMF, or DMSO along with an organic base such as triethylamine (TEA) or DIEA stirred at a temperature of 0 to 70° C., preferably at about 23° C. (room temperature).

In Formula I, when R₁ is an amine, it can react with 2,2,2-trichloroethyl carbonochloridate in pyridine from 0° C. to room temperature, then transform further with a secondary amine and 1,8-diazabicyclo[5.4.0]undec-7-ene (DBU) in THF from 40-60° C. to form a urea at R₃. In Formula I, when R₁ is phenyl carbamate, it can react with an amine and a base like TEA in a solvent like DMF from 40° C., then deprotected under acidic conditions with HCl in a solvent like dioxane and DCM from 0° C. to room temperature to form a urea at R₃. In Formula I, when R₃ is a tert-butyl acetate, it can be deprotected under standard acidic conditions, such as HCl in a solvent like H₂O and DCM. In Formula I, when R₁ is an amine, it can react with ethyl carbonochloridate with a base like TEA in a solvent like DCM from 0° C. to room temperature to form an amide at R₃.

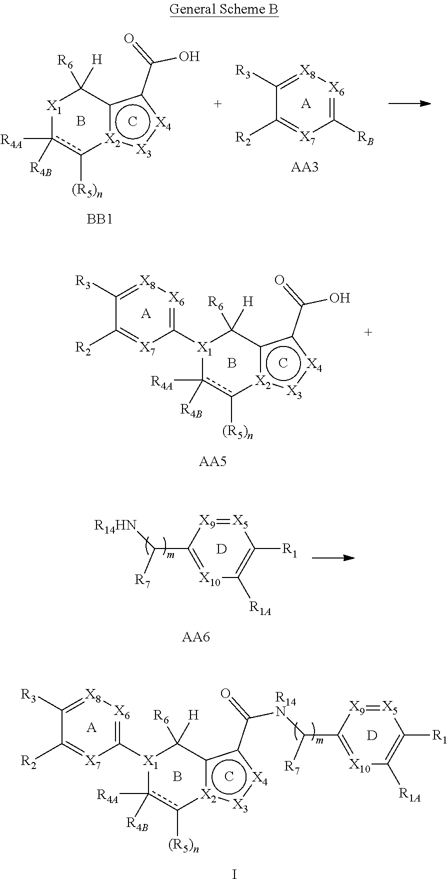

The general structure of Formula I can be prepared as shown in General Scheme B. The amine of Formula BB1 can be treated with a halogen (e.g., R_{B}═F or Cl)-substituted aromatic of Formula AA3 using standard SNAr conditions such as cesium fluoride (CsF) or K₃PO₄ in a solvent like water or DMSO and stirred at 80 to 140° C. to afford Formula AA5. The compound of Formula I can be obtained by reacting the carboxylic acid of AA5 with the amine of Formula AA6 under standard amide coupling reagents like EDCl with HOPO, HATU, or other common amide coupling reagents in solvents such as ACN and H₂O, DMF, or DMSO along with an organic base such as TEA or DIEA stirred at a temperature of 0 to 70° C., preferably at room temperature. In Formula I, when R₆ is a hydrogen, can be oxidized with 2,3-dichloro-5,6-dicyano-p-benzoquinone (DDQ) in acetone and then further transformed to methylate when reacted with boron trifluoride diethyl etherate (BF₃OEt₂) and dimethylzinc in solvents like toluene and THF. In Formula I, when R₃ is a tert-butyl acetate, can be deprotected under standard acidic conditions, such as HCl in a solvent like dioxane, H₂O, and DCM, then transformed further under standard amide coupling conditions to form an amide at R₃.

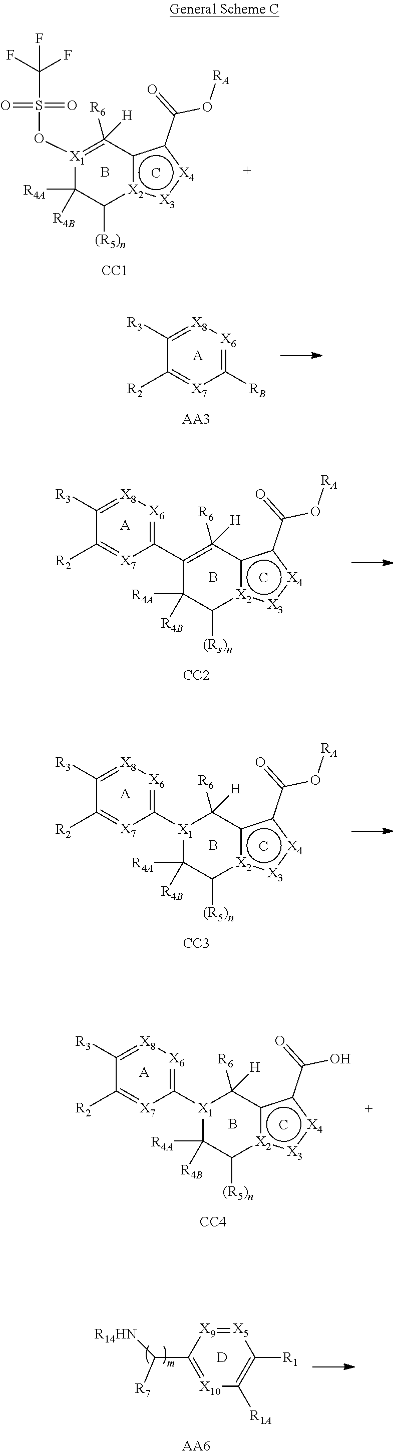

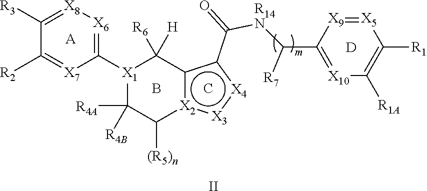

The general structure of Formula II can be prepared as shown in General Scheme C. The trifluoromethanesulfonic acid of Formula CC1, synthesized by literature methods or purchased commercially, can be treated with a boronic acid (e.g. R_{B}═B—(OH)₂)-substituted aromatic of Formula AA3 using with a Pd catalyst such as Pd(dppf)Cl₂ and a base like K₃PO₄ in solvents such as H₂O and THF at 85° C. to form Formula CC2. The alkene of Formula CC2 can undergo hydrogenation with palladium on carbon (Pd/C) in EtOH to reduce the alkene to form Formula CC3. The ester (e.g., R_{A}=ethyl) of Formula CC3 can undergo standard hydrolysis conditions by utilizing a base like LiOH in solvents like THF, MeOH and H₂O from 0 to 50° C., preferably at room temperature, to afford the carboxylic acid of Formula CC4. The compound of Formula II can be obtained by reacting the carboxylic acid of CC4 with the amine of Formula AA6 under standard amide coupling reagents like EDCl with HOPO in solvents such as DMSO along with an organic base such as DIEA stirred at 0 to 70° C. II can be prepared as shown in General Scheme C. The trifluoromethanesulfonic acid of Formula CC1, synthesized by literature methods or purchased commercially, can be treated with a boronic acid (e.g. R_{B}═B—(OH)₂)-substituted aromatic of Formula AA3 using with a Pd catalyst such as Pd(dppf)Cl₂ and a base like K₃PO₄ in solvents such as H₂O and THF at 85° C. to form Formula CC2. The alkene of Formula CC2 can undergo hydrogenation with palladium on carbon (Pd/C) in EtOH to reduce the alkene to form Formula CC3. The ester (e.g., R_{A}=ethyl) of Formula CC3 can undergo standard hydrolysis conditions by utilizing a base like LiOH in solvents like THF, MeOH and H₂O from 0 to 50° C., preferably at room temperature, to afford the carboxylic acid of Formula CC4. The compound of Formula II can be obtained by reacting the carboxylic acid of CC4 with the amine of Formula AA6 under standard amide coupling reagents like EDCl with HOPO in solvents such as DMSO along with an organic base such as DIEA stirred at 0 to 70° C.

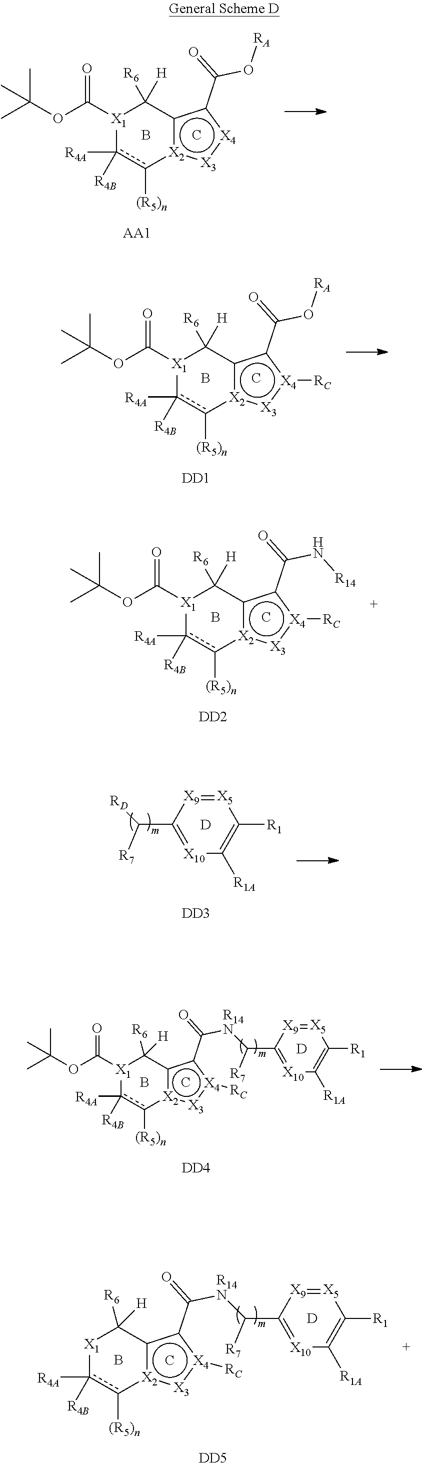

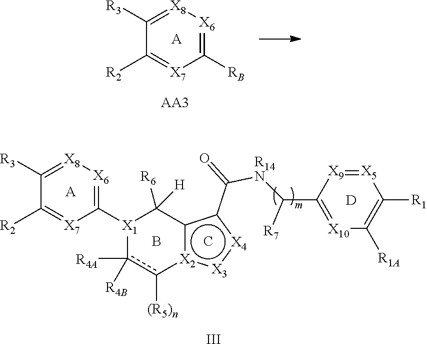

The general structure of Formula III can be prepared as shown in General Scheme D. The BOC protected amine of Formula AA1, synthesized by literature methods or purchased commercially, which can be treated with bromo substituted alkyl amine protected as benzyloxy carbonyl (Cbz) with a base like Cs₂CO₃ in ACN to give mixture of N1 and N2-alkylated (e.g. R_{C}=Cbz protected alkyl amine) compounds of Formula DD1. The regioisomers can be separated by silica gel column purification to provide compounds of Formula DD1. Formula DD1 can then undergo deprotection of the CBz protecting group with reducing conditions such as Pd/C in a solvent like EtOH at 50-120 pounds per square inch (psi) of hydrogen (H₂) gas. The free amine on the N2 of Formula DD1 can be reacted with DBU in a solvent like dioxane at 100° C. to cyclize to form a C₆₇ heterocycle fused to Ring C of Formula DD2. The cyclized lactam of Formula DD2 can be treated with a halogen (e.g., R_{D}═Br)-substituted aromatic of Formula DD3 with bases such as K₂CO₃, potassium bis(trimethylsilyl)amide (KHMDS) in THF with additional solvents such as ACN and DMF with stirring at 80° C. to afford Formula DD4. The BOC protected amine of Formula DD4 can be deprotected under acidic conditions such as HCl in solvents like dioxane and DCM to form the amine of Formula DD5. The amine of Formula DD5 can be treated with a halogen (e.g., R_{B}═F)-substituted aromatic of Formula AA3 using standard SNAr conditions such as K₃PO₄ in a solvent like tert-amyl alcohol and H₂O with stirring at 100° C. to afford Formula III. In Formula III, when R₁ is an iodine, can react further with an urea with a base like Cs₂CO₃ and palladium catalyst such as ((SP-4-3)-[[5-(diphenylphosphino)-9,9-dimethyl-9H-xanthen-4-yl]diphenylphosphine-κP](methanesulfonato-κO)[2′-(methylamino-κN)[1,1′-biphenyl]-2-yl-κC]-palladium) XantPhos Pd G4 in a solvent like tert-amyl alcohol at 90° C. to form an urea at R₁ in Formula III.

In order that this disclosure may be better understood, the following examples are set forth. These examples are for the purpose of illustration only and are not to be construed as limiting the scope of the disclosure in any manner.

### EXAMPLES

The compounds and intermediates described below were named using the naming convention provided with ChemDraw version 20.1.1.123. The naming convention provided with ChemDraw version 20.1.1.123 is well known by those skilled in the art and it is believed that the naming convention generally comports with the IUPAC (International Union for Pure and Applied Chemistry) recommendations on Nomenclature of Organic Chemistry and the CAS Index rules. Unless noted otherwise, all reactants were obtained commercially without further purifications or were prepared using methods known in the literature.

The following illustrates the synthesis of various compounds of the present invention. Additional compounds within the scope of this invention may be prepared using the methods illustrated in these Examples, either alone or in combination with techniques generally known in the art.

All starting materials in these Preparations and Examples are either commercially available or can be prepared by methods known in the art or as described herein.

Commercial solvents and reagents were generally used without further purification. Anhydrous solvents were employed where appropriate, generally ACROSEAL™ products from Acros Organics, Aldrich SURE/SEAL™ from Sigma-Aldrich, or DRISOLV™ products from EMD Chemicals. Commercial solvents and reagents were used without further purification.

Experiments were generally carried out under inert atmosphere (nitrogen or argon), particularly in cases where oxygen- or moisture-sensitive reagents or intermediates were employed.

Unless otherwise noted, chemical reactions were performed at room temperature (about 23 degrees Celsius).

In some examples, chiral separations were carried out to separate enantiomers or diastereomers of certain compounds of the disclosure.

For syntheses referencing procedures in other Examples or Methods, reaction conditions (reaction time and temperature) may vary. In general, reactions were followed by thin-layer chromatography (TLC) or mass spectrometry (MS) and subjected to work-up when appropriate.

Commercial benchtop photoreactors such as the Penn OC Photoreactor M2, Acceled Photoreactor M2 and Lumidox II with 24-well block were utilized with 450 or 445 nanometer (nm) light-emitting diode (LED).

Purifications may vary between experiments: in general, solvents and the solvent ratios used for eluents/gradients were chosen to provide appropriate retention times.

Reaction progress was monitored using thin layer chromatography (TLC), liquid chromatography-mass spectrometry (LCMS) and high-performance liquid chromatography (HPLC), analyses. TLC was performed on pre-coated silica gel plates with a fluorescence indicator (254 nm excitation wavelength) and visualized under ultraviolet (UV) light.

LCMS data were acquired on an Agilent 1100 Series instrument with a Leap Technologies autosampler, Gemini C₁₈ columns, ACN/water gradients, and either trifluoroacetic acid (TFA), formic acid, or ammonium hydroxide (NH₄OH) modifiers or similar equipment. The column eluent was analyzed using Waters ZQ mass spectrometer scanning in both positive and negative ion modes from 100 to 1200 Da. Other similar instruments were also used.

High resolution mass spectrometry (HR/MS) was gathered on Sciex TripleTOF 5600+ with DuoSpray ionization source with the liquid chromatography instrument with an Agilent Technologies, 1200 binary pump, Agilent 1200 autosampler, Agilent 1200 column compartment, and Agilent 1200 diode array detector. The instrument acquisition and data handling were done with Sciex Analyst TF version 1.7.1.

HPLC data were acquired on an Agilent 1100 Series instrument using Gemini, Sunfire, Welch Boltimate™, Welch Xtimate, Prep PG-45 MOD10, Boston Prime, Boston Green ODS, Phenomenex Gemini NX or XBridge C₁₈ columns with acetonitrile (ACN)/water (H₂O) gradients, and either TFA, formic acid, NH₄OH, or ammonium bicarbonate (NH₄HCO₃) modifiers and comparable equipment. Purifications were performed by high performance liquid chromatography (HPLC) and medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges and the like.

Chiral purifications were performed by chiral supercritical fluid chromatography (SFC) using Berger or Thar instruments and similar instruments with DAICEL CHIRALCEL OD, OJ; DAICEL CHIRALPAK AD, AS, IF; Chiral Technologies OJ-H, AD-H, OD-H, IA, IB; Lux Cellulose 1; Lux Cellulose 3; Pirkle Covalent (R,R) Whelk-O1; CHIRALPAK IH; YMC-IB; and Phenomenex Lux Cellulose 1 columns; and carbon dioxide (CO₂) mixtures with methanol (MeOH), ethanol (EtOH), isopropyl alcohol, or ACN, alone or modified using TFA, formic acid, NH₄OH, diethylamine (DEA), ammonia (NH₃) or isopropylamine. UV detection was used to trigger fraction collection.

The chiral purity was determined using supercritical fluid chromatography (SFC) and reverse-phase liquid chromatography (RPLC) analytical method with Chiralpak AD-3, IG-3, IF, AS-3, IB—N; Chiralcel OJ-, OD-3; Chiral Technologies OJ-H, AD-H, OD-H, IA, IB, IH; Lux Cellulose 1; IM-3; Lux Cellulose 3; Phenomenex Kinetex and Regis (R,R) Whelk-01 columns; and CO₂ mixtures with MeOH, EtOH, isopropyl alcohol, or ACN, alone or modified using TFA, formic acid, NH₄OH, DEA, NH₃, or isopropylamine.

Proton nuclear magnetic resonance (¹H NMR) spectra were recorded, referenced to residual peaks from the deuterated solvents employed, where b is chemical shift; d is doublet; dd is doublet of doublets; ddd is doublet of doublet of doublets; dt is doublet of triplets, m is multiplet, s is singlet, t is triplet, q, quartet; quin, quintet, br s, broad singlet; MHz is megahertz, ppm is parts per million. Proton nuclear magnetic spectroscopy (¹H NMR) chemical shifts are given in parts per million (ppm, δ) referenced to the deuterated solvent downfield from tetramethylsilane and were recorded on 300, 400, 500, or 600 MHz Varian spectrometers.

Products were generally dried under vacuum before being carried on to further reactions or submitted for biological testing.

In the experimental sections that follow the following abbreviations may be used. ACN is acetonitrile; abs in the structures is absolute and refers to the stereochemistry being confirmed as R or S; AcOH is acetic acid; APhos Pd G3 is [4-(di-tert-butylphosphino)-N,N-dimethylaniline-2-(2′-aminobiphenyl)]palladium(II) methanesulfonate; BBr₃ is boron tribromide; B₂Pin₂ is bis(pinacolato)diboron; BOC is tert-butyloxycarbonyl; Boc₂O is di-tert-butyl dicarbonate; brine is a saturated sodium chloride water solution; BINAP is (±)-2,2′-bis(diphenylphosphino)-1,1′-binaphthalene; BF₃OEt₂ is boron trifluoride diethyl etherate; ° C. is degrees Celsius; CDCl₃ is deuterated chloroform; CD₃OD is deuterated methanol; (CD₃)₂SO is deuterated dimethyl sulfoxide; cataCXium® A is di(1-adamantyl)-n-butylphosphine; cataCXium® A Pd G3 is mesylate[(di(1-adamantyl)-n-butylphosphine)-2-(2′-amino-1,1′-biphenyl)]palladium(II); CAS is Chemical Abstracts Service; CDI is 1,1′-carbonyldiimidazole; CHCl₃ is chloroform; CO₂ is carbon dioxide; CsHCO₃ is cesium hydrogencarbonate; Cs₂CO₃ is cesium carbonate; CsF is cesium fluoride; CuI is copper(I) iodide; DBU is 1,8-diazabicyclo[5.4.0]undec-7-ene; DCM is dichloromethane; DCE is 1,2-dichloroethane; DDQ is 2,3-dichloro-5,6-dicyano-p-benzoquinone; DEA is diethylamine; DIEA is N,N-diisopropylethylamine; dioxane is 1,4-dioxane; DMA is dimethylacetamide; DMAP is 4-(dimethylamino)pyridine; DME is dimethyl ether; DMEDA is 1,2-dimethylethylenediamine; DMF is N,N-dimethylformamide; DMSO is dimethyl sulfoxide; EDCl is 1-(3-dimethylaminopropyl)-3-ethylcarbodiimide hydrochloride; ee is enantiomeric excess; EtOAc is ethyl acetate; EtOH is ethanol; g is gram; g/L is grams per liter; h is hour; HATU is 1-[bis(dimethylamino)methylene]-1H-1,2,3-triazolo[4,5-b]pyridinium 3-oxid hexafluorophosphate; HCl is hydrochloric acid; H₂ is hydrogen; H₂O is water; HOBt is 1-hydroxybenzotriazole hydrate; HOPO is 2-pyridinol 1-oxide; HPLC is high performance liquid chromatography; HR/MS is high resolution mass spectrometry; Hz is hertz; Ir(ppy)₂(dtbbpy)PF₆ is [4,4′-bis(1,1-dimethylethyl)-2,2′-bipyridine-N1,N1′]bis[2-(2-pyridinyl-N)phenyl-C]iridium(II) hexafluorophosphate; KHSO₄ is potassium bisulfate; KHMDS is potassium bis(trimethylsilyl)amide; KOAc is potassium acetate; KOH is potassium hydroxide; KOTMS is potassium trimethylsilanolate; K₂CO₃ is potassium carbonate; kg is kilogram; K₃PO₄ is tripotassium phosphate; KH₂PO₄ is potassium phosphate monobasic; L is liter; LC is liquid chromatography; LCMS is liquid chromatography mass spectrometry; LDA is lithium diisopropylamide; LED is light-emitting diode; LiOH is lithium hydroxide monohydrate; M is molar; LiHMDS is lithium bis(trimethylsilyl)amide; Mel is iodomethane; MeOH is methanol; 4MetBuXPhos Pd G3 is methanesulfonato (2-di-tert-butylphosphino-3,4,5,6-tetramethyl-2′,4′,6′-triisopropyl-1,1-biphenyl)(2′-amino-1,1′-biphenyl-2-yl) palladium(II); mg is milligram; MgSO₄ is magnesium sulfate; MPLC is medium performance liquid chromatography; MHz is megahertz; min is minute; mL is milliliter; mL/min is milliliter per minute; mmol is millimole; mol is mole; mM is millimolar; mm is millimeter; MPa is megapascal; MS is mass spectrometry; MTBE is methyl tertiary-butyl ether; m/z is mass-to-charge ratio; N is normal (concentration); N₂ is nitrogen; NBS is N-bromosuccinimide; NaBH₄ is sodium borohydride; ND is not determined; N-XantPhos Pd G3 is (2′-amino-2-biphenylyl)(methanesulfonato-κO)palladium-4,6-bis(diphenylphosphino)-10H-phenoxazine; NH₃ is ammonia; Na₂CO₃ is sodium carbonate; NaH is sodium hydride; NaHCO₃ is sodium bicarbonate; NaI is sodium iodide; NaOCH₃ is sodium methoxide; NaOH is sodium hydroxide; Na₂SO₄ is sodium sulfate; NaOtBu is sodium tert-butoxide; n-BuLi is n-butyllithium; NH₄Cl is ammonium chloride; NH₄HCO₃ is ammonium bicarbonate; NH₄OH is ammonium hydroxide; NMR is nuclear magnetic resonance; NiCl₂·glyme is nickel(II) chloride ethylene glycol dimethyl ether complex; NiBr₂·DME is nickel(II) bromide ethylene glycol dimethyl ether complex; P(t-Bu)₃ Pd G2 is chloro[(tri-tert-butylphosphine)-2-(2-aminobiphenyl)]palladium(II); Pd(dppf)Cl₂ is (1,1′-bis(diphenylphosphino)ferrocene)palladium(II) dichloride; Pd/C is palladium on carbon; Pd₂(dba)₃ is tris(dibenzylideneacetone)dipalladium(0); PdCl₂(PPh₃)₂ is bis(triphenylphosphine)palladium(II) dichloride; Pd(OAc)₂ is palladium(II) acetate; Pd(t-Bu₃P)₂ is bis(tri-tert-butylphosphine)palladium(0); PE is petroleum ether; psi is pounds per square inch; quinuclidine is 1-azabicyclo[2.2.2]octane; SFC is super critical fluid chromatography; SOCl₂ is thionyl chloride; TBD is 1,5,7-triazabicyclo[4.4.0]dec-5-ene; TEA is triethylamine; tert-amyl alcohol is 2-methyl-2-butanol; TFA is trifluoroacetic acid; THF is tetrahydrofuran; TLC is thin-layer chromatography; trimethylboroxine is 2,4,6-trimethyl-1,3,5,2,4,6-trioxatriborinane; μm is micrometer; μmol is micromole; rac is racemic referring to the compound including a mixture of compounds having both R and S stereochemistry and is denoted in the structures as “&1”; rel is relative referring to the stereochemistry being either R or S and is denoted in the structures as “or1” or as an asterisk in the named structure if there are two chiral centers in the compound; RhCl(PPh₃)₃ is tris(triphenylphosphine)rhodium(I) chloride; rpm is revolutions per minute; RPLC is reverse-phase liquid chromatography; Ru/Al₂O₃ is ruthenium on aluminum oxide; RuPhos is 2-dicyclohexylphosphino-2′,6′-diisopropoxybiphenyl; RuPhos Pd G3 is (2-dicyclohexylphosphino-2′,6′-diisopropoxy-1,1′-biphenyl)[2-(2′-amino-1,1′-biphenyl)]palladium(II) methanesulfonate; RuPhos Pd G4 is [dicyclohexyl(2′,6′-diisopropoxy-2-biphenylyl)phosphine-κP](methanesulfonatato-κO)[2′-(methylamino-κN)-2-biphenylyl-κC²]palladium; RuCl(PPh₃)₃ Ru/SiO₂ is ruthenium on silica; wt is weight; XPhos Pd G2 is chloro(2-dicyclohexylphosphino-2′,4′,6′-triisopropyl-1,1′-biphenyl)[2-(2′-amino-1,1′-biphenyl)]palladium(II); XantPhos Pd G4 is (SP-4-3)-[[5-(diphenylphosphino)-9,9-dimethyl-9H-xanthen-4-yl]diphenylphosphine-κP](methanesulfonato-κO)[2′-(methylamino-κN)[1,1′-biphenyl]-2-yl-κC]-palladium; and Zn is zinc.

### Preparation 1

### Ethyl 1-cyclopropyl-4,5,6,7-tetrahydro-1H-pyrazolo[4,3-c]pyridine-3-carboxylate hydrochloride (P1)

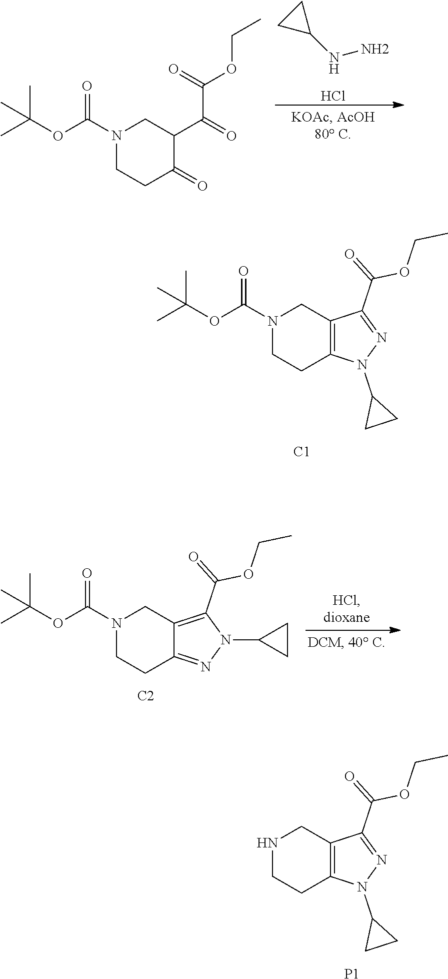

### Step 1. Preparation of 5-(tert-butyl) 3-ethyl 1-cyclopropyl-1,4,6,7-tetrahydro-5H-pyrazolo[4,3-c]pyridine-3,5-dicarboxylate (C1) and 5-(tert-butyl) 3-ethyl 2-cyclopropyl-2,4,6,7-tetrahydro-5H-pyrazolo[4,3-c]pyridine-3,5-dicarboxylate (C2)

A reaction containing cyclopropylhydrazine monohydrochloride (36.3 mg, 0.334 mmol) and KOAc (36.1 mg, 0.367 mmol) in AcOH (1.0 mL) was heated at 80° C. for 10 min then cooled to room temperature. tert-butyl 3-(2-ethoxy-2-oxoacetyl)-4-oxopiperidine-1-carboxylate (CAS: 518990-24-4; 0.100 g, 0.334 mmol) was added to the mixture. The reaction turned into a viscous yellow oil, which was stirred at 80° C. for 1 h and 30 min. The reaction was then diluted with EtOAc and saturated aqueous NaHCO₃. The organic layer was separated and concentrated in vacuo. The residue was purified by column chromatography (silica gel, 0-40% EtOAc:heptane) to provide minor first eluting isomer C2: (11 mg, 27.2% yield) (LC/MS) m/z (M+H)⁺=336.3; followed by major second eluting isomer C1: (18 mg, 44.5% yield) (LC/MS) m/z (M+H)⁺=336.3; ¹H NMR (600 MHz, CDCl₃) δ 4.66-4.54 (m, 2H), 4.36 (q, 2H), 3.71 (s, 2H), 3.40-3.34 (m, 1H), 2.84-2.74 (m, 2H), 1.48-1.43 (m, 9H), 1.41-1.34 (m, 3H), 1.21-1.18 (m, 2H), 1.07-1.02 (m, 2H).

### Step 2. Preparation of ethyl 1-cyclopropyl-4,5,6,7-tetrahydro-1H-pyrazolo[4,3-c]pyridine-3-carboxylate hydrochloride (P1)

A solution of C1 (0.200 g, 0.596 mmol) was dissolved in DCM (2.0 mL), then 4M HCl in dioxane (0.130 g, 3.58 mmol) was added, then the reaction was stirred at 40° C. for 25 min. The reaction was concentrated in vacuo to provide P1 (0.168 mg, crude) as a white solid. The solid was used in the next step without further purification. (LC/MS) m/z (M+H)⁺=236.2; ¹H NMR (600 MHz, CD₃OD) δ 4.41-4.33 (m, 4H), 3.61-3.53 (m, 3H), 3.16 (t, 2H), 1.37 (t, 3H), 1.21-1.16 (m, 2H), 1.15-1.09 (m, 2H).

### Preparation 2

### Ethyl 1-isopropyl-4,5,6,7-tetrahydro-1H-pyrazolo[4,3-c]pyridine-3-carboxylate hydrochloride (P2)

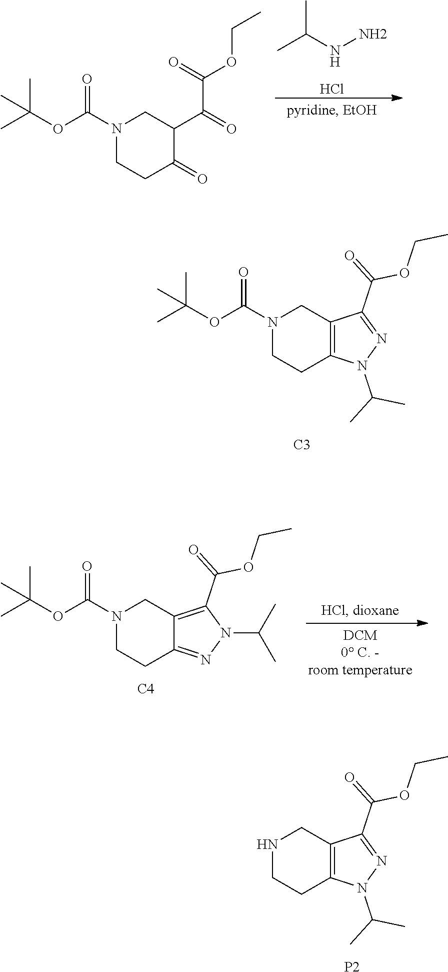

### Step 1. Preparation of 5-(tert-butyl) 3-ethyl 1-isopropyl-1,4,6,7-tetrahydro-5H-pyrazolo[4,3-c]pyridine-3,5-dicarboxylate (C3) and 5-(tert-butyl) 3-ethyl 2-isopropyl-2,4,6,7-tetrahydro-5H-pyrazolo[4,3-c]pyridine-3,5-dicarboxylate (C4)

To a solution of tert-butyl 3-(2-ethoxy-2-oxoacetyl)-4-oxopiperidine-1-carboxylate (CAS: 518990-24-4, 325 g, 1.09 mol) in EtOH (2.3 L) was added isopropylhydrazine hydrochloride (120 g, 1.1 mol) in one portion then pyridine (103 g, 1.30 mol) was added dropwise into the mixture for 10 min at 20-25° C. with ice water bath. The reaction mixture was stirred for 2 h at room temperature. The reaction was carried out in two batches in parallel, were combined, and then concentrated in vacuo. The residue was diluted with EtOAc (5 L) and washed with brine (2×5 L) then dried with Na₂SO₄, filtered, and concentrated in vacuo. The residue was purified by column chromatography (silica gel, EtOAc:PE from 10-50%) to provide C3 and C4. C3 was dissolved in PE (0.8 L) and then cooled to −20° C. The mixture was stirred for 4 h, resulting solids were filtered, and washed with PE (0.1 L). The filter cake was dried under high vacuum to obtain C3 (414 g, 55.9% yield) as a white solid. (LC/MS) m/z (M+H)⁺=338.1; ¹H NMR (400 MHz, CDCl₃) δ 4.59 (s, 2H), 4.48-4.32 (m, 3H), 3.71 (t, 2H), 2.70 (t, 2H), 1.53-1.45 (m, 15H), 1.39-1.34 (m, 3H). C4 was dissolved in PE (50 mL) then cooled to −20° C. The mixture was stirred for 4 h, resulting solids were filtered, and washed with PE (20 mL). The filter cake was dried under high vacuum to provide C4 (11.7 g, 1.58% yield) as a white solid. (LC/MS) m/z (M+H)⁺=338.1; ¹H NMR (400 MHz, CDCl₃) δ 5.60-5.43 (m, 1H), 4.60 (s, 2H), 4.33 (q, 2H), 3.68 (s, 2H), 2.75 (s, 2H), 1.50-1.44 (m, 15H), 1.38 (m, 3H).

### Step 2. Preparation of ethyl 1-isopropyl-4,5,6,7-tetrahydro-1H-pyrazolo[4,3-c]pyridine-3-carboxylate hydrochloride (P2)

To a mixture of C3 (13.5 g, 40.0 mmol) in DCM (60 mL) was added 4N HCl in dioxane (150 mL) in batches at 0° C. The reaction was stirred at about 15° C. for 16 h and then concentrated in vacuo to provide P2 (10.5 g, crude) as a white solid, used directly in the next step without further purification. LC/MS m/z (M+H)⁺=238.1; ¹H NMR (600 MHz, (CD₃)₂SO) δ 9.80 (s, 1H), 4.63-4.51 (m, 1H), 4.28 (q, 2H), 4.18 (s, 2H), 3.36 (s, 2H), 3.02 (t, 2H), 1.38 (d, 6H), 1.29 (t, 3H).

### Preparation 3

### 5-(tert-Butoxycarbonyl)-1-ethyl-4,5,6,7-tetrahydro-1H-pyrazolo[4,3-c]pyridine-3-carboxylic acid (P3)

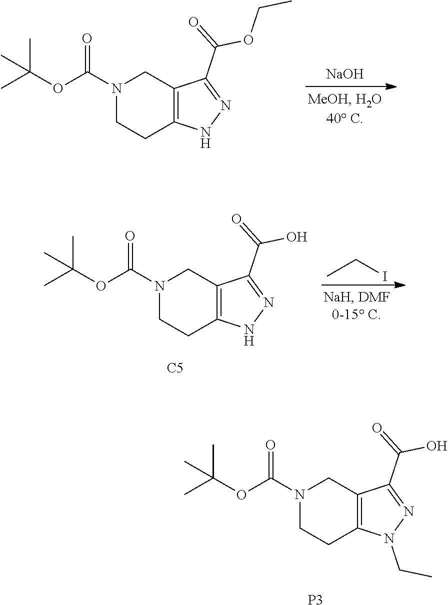

### Step 1. Preparation of 5-(tert-butoxycarbonyl)-4,5,6,7-tetrahydro-1H-pyrazolo[4,3-c]pyridine-3-carboxylic acid (C5)

To a suspension of 5-(tert-butyl) 3-ethyl 1,4,6,7-tetrahydro-5H-pyrazolo[4,3-c]pyridine-3,5-dicarboxylate (CAS: 518990-23-3; 2.00 g, 6.77 mmol) in MeOH (7.7 mL) and H₂O (7.7 mL) was added NaOH (0.542 g, 13.5 mmol). The reaction mixture was stirred at 40° C. for 16 h. The mixture was diluted with H₂O (20 mL), cooled to 0° C. and acidified with 1M aqueous HCl to pH of about 3. The resulting mixture was filtered, the solid was dried further under a high vacuum and then lyophilized to provide C5 (1.60 g, crude) as a white solid. The solid was used directly in the next step without further purification. (LC/MS) m/z (M+H)⁺=268.1. ¹H NMR (400 MHz, (CD₃)₂SO) δ 13.17 (br s, 2H), 4.48 (s, 2H), 3.58 (t, 2H), 2.65 (t, 2H), 1.41 (s, 9H).

### Step 2. Preparation of 5-(tert-butoxycarbonyl)-1-ethyl-4,5,6,7-tetrahydro-1H-pyrazolo[4,3-c]pyridine-3-carboxylic acid (P3)

To the suspension of C5 (0.200 g, 0.748 mmol) in DMF (3.5 mL) was added NaH (89.8 mg, 2.24 mmol, 60%) at 0° C. and stirred for 30 min at 0° C. A solution of iodoethane (0.140 g, 0.898 mmol) in DMF (0.5 mL) was added at 0° C. then stirred at 15° C. for about 4 h. An additional portion of the solution of iodoethane (23.3 mg, 0.150 mmol) in DMF (0.2 mL) was added to the mixture and stirred at 15° C. for 16 h. The reaction solution was quenched with H₂O (10 mL) and then extracted with EtOAc (2×20 mL). The aqueous layer was acidified with 1M aqueous HCl to pH ˜3 and then extracted with EtOAc (2×30 mL). The combined organic layers were washed with brine (2×30 mL), dried over Na₂SO₄, concentrated in vacuo and then lyophilized to provide P3 (0.173 g, 78.5% yield) as a white solid. (LC/MS) m/z (M+H)⁺=296.1. ¹H NMR (400 MHz, (CD₃)₂SO) δ 12.64 (br s, 1H), 4.45 (s, 2H), 4.07 (q, 2H), 3.60 (t, 2H), 2.74-2.65 (m, 2H), 1.41 (s, 9H), 1.31 (t, 3H).

### Preparation 3b

### Ethyl 1-ethyl-4,5,6,7-tetrahydro-1H-pyrazolo[4,3-c]pyridine-3-carboxylate hydrochloride (P3b)

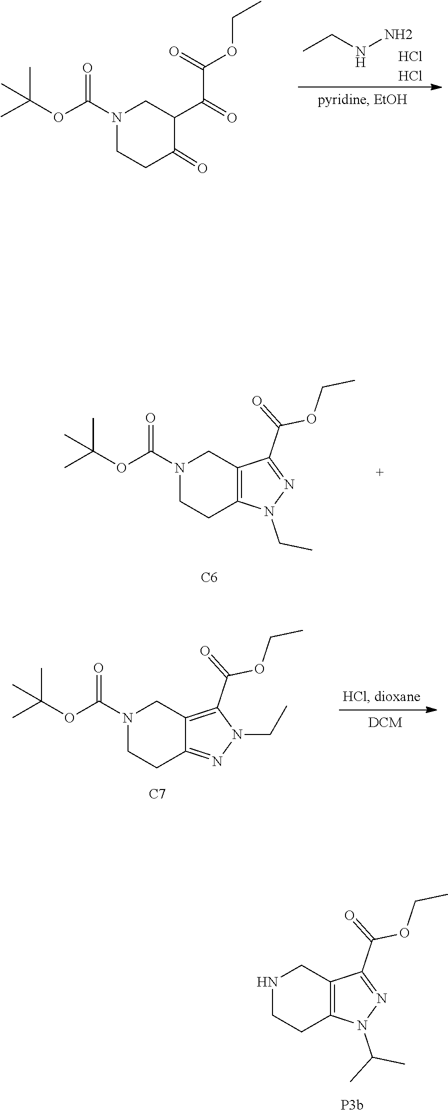

### Step 1. Preparation of 5-(tert-butyl) 3-ethyl 1-ethyl-1,4,6,7-tetrahydro-5H-pyrazolo[4,3-c]pyridine-3,5-dicarboxylate (C6) and 5-(tert-butyl) 3-ethyl 2-ethyl-2,4,6,7-tetrahydro-5H-pyrazolo[4,3-c]pyridine-3,5-dicarboxylate (C7)

To a solution of tert-butyl 3-(2-ethoxy-2-oxoacetyl)-4-oxopiperidine-1-carboxylate (CAS: 518990-24-4; 97.5 g, 326 mmol) and ethylhydrazine dihydrochloride (56.3 g, 423 mmol) in EtOH (1.6 L) was added pyridine (129 g, 1.63 mol). The reaction mixture was stirred at room temperature for 16 h then concentrated in vacuo. The residue was purified by column chromatography (silica gel, 0-50% THF:PE) to provide the first eluting isomer C6 (58.0 g) as a white solid; followed by the second eluting isomer C7 (20.6 g, 19.6% yield) as a light-yellow oil. C6 was diluted with EtOAc (200 mL) then stirred at 50° C. for 30 min and cooled to 0° C. which caused white solid precipitate to form. The suspension was filtered, and the filter cake was washed with EtOAc (3×10 mL). The filter cake was collected and concentrated in vacuo to provide C6 (35.4 g, 33.6% yield) as a white solid.

C6: (LC/MS) m/z (M+H)⁺=324.2; ¹H NMR (400 MHz, CDCl₃) δ 4.60 (s, 2H), 4.38 (q, 2H), 4.12 (q, 2H), 3.71 (t, 2H), 2.68 (t, 2H), 1.47 (s, 9H), 1.44-1.34 (m, 6H).

C7: (LC/MS) m/z (M+H)⁺=324.3; ¹H NMR (400 MHz, CDCl₃) δ 4.65-4.50 (m, 4H), 4.34 (q, 2H), 3.73-3.63 (m, 2H), 2.80-2.67 (m, 2H), 1.48 (s, 9H), 1.43-1.33 (m, 6H).

### Step 2. Preparation of ethyl 1-ethyl-4,5,6,7-tetrahydro-1H-pyrazolo[4,3-c]pyridine-3-carboxylate hydrochloride (P3b)

To a solution of C6 (1.00 g, 3.09 mmol) in DCM (10 mL) was added 2M HCl in dioxane (1.13 g, 30.9 mmol). The reaction mixture was stirred at room temperature for 2.5 h then concentrated in vacuo to provide P3b (0.870 g, crude) as a white solid. The solid was used directly in the nest step without further purification. (LC/MS) m/z (M+H)⁺=224.2. ¹H NMR (400 MHz, CD₃OD) δ 4.44-4.32 (m, 4H), 4.20 (q, 2H), 3.56 (t, 2H), 3.09 (t, 2H), 2.80-2.67 (m, 2H), 1.44 (t, 3H), 1.38 (t, 3H).

### Preparation 4

### Ethyl 1-methyl-4,5,6,7-tetrahydro-1H-pyrazolo[4,3-c]pyridine-3-carboxylate hydrochloride (P4)

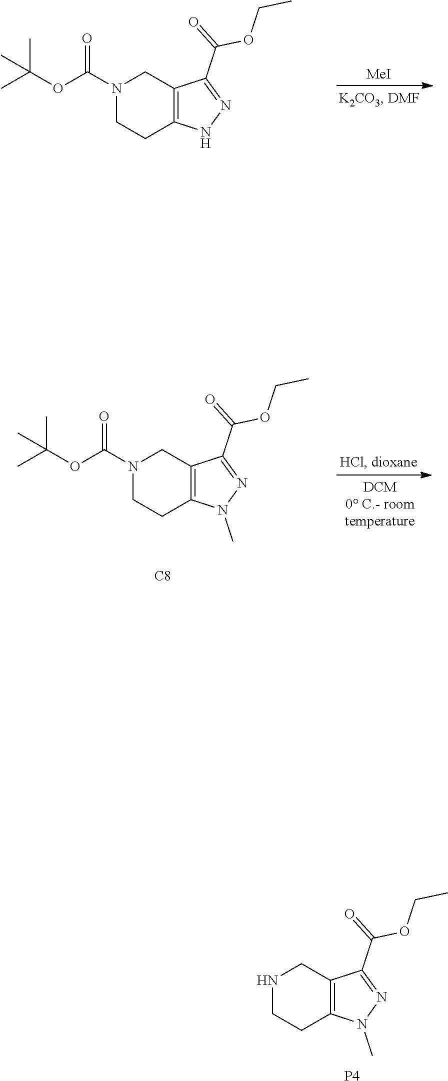

### Step 1. Preparation of 5-(tert-butyl) 3-ethyl 1-methyl-1,4,6,7-tetrahydro-5H-pyrazolo[4,3-c]pyridine-3,5-dicarboxylate (C8)

5-(tert-Butyl) 3-ethyl 1,4,6,7-tetrahydro-5H-pyrazolo[4,3-c]pyridine-3,5-dicarboxylate (CAS: 518990-23-3; 2.00 g, 6.77 mmol) was dissolved in DMF (10.0 mL) then K₂CO₃ (2.81 g, 20.3 mmol) and Mel (0.961 g, 6.77 mmol) were added. The reaction was stirred at room temperature for 3 h and diluted with EtOAc and H₂O. The organic layer was separated and then concentrated in vacuo. The residue was dissolved in DCM and then purified by column chromatography (silica gel, 0-40% EtOAc:heptane) to provide C8 (0.825 g, 39.4% yield) as a clear oil. (LC/MS) m/z (M+H)⁺=310.3; ¹H NMR (600 MHz, CDCl₃) δ 4.61 (s, 2H), 4.35 (q, 2H), 4.14 (s, 3H), 3.68 (s, 2H), 2.74 (s, 2H), 1.49 (s, 9H), 1.41-1.37 (m, 3H).

### Step 2. Preparation of ethyl 1-methyl-4,5,6,7-tetrahydro-1H-pyrazolo[4,3-c]pyridine-3-carboxylate hydrochloride (P4)

C8 (2.00 g, 6.46 mmol) was dissolved in 4M HCl in dioxane (40.0 mL) at 0° C. and then stirred at room temperature for 16 h. The reaction was concentrated in vacuo to provide P4 (1.30 g, 81.9% yield) as a yellow solid. This was used in the next step without further purification. (LC/MS) m/z (M+H)⁺=210.3. ¹H NMR (400 MHz, CDCl₃) δ 10.23 (br s, 1H), 4.50-4.32 (m, 4H), 3.86 (s, 3H), 3.55-3.46 (m, 2H), 3.13 (t, 2H), 1.40 (t, 3H).

### Preparation 5

### Ethyl 3-cyclopropyl-5,6,7,8-tetrahydroimidazo[1,5-a]pyrazine-1-carboxylate hydrochloride (P5)

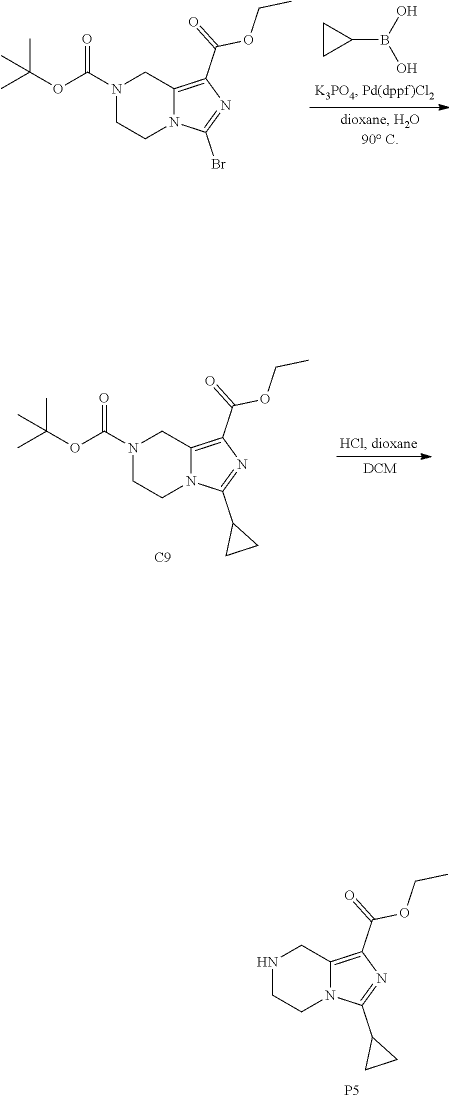

### Step 1: Preparation of 7-(tert-butyl) 1-ethyl 3-cyclopropyl-5,6-dihydroimidazo[1,5-a]pyrazine-1,7(8H)-dicarboxylate (C9)

To a solution of 7-(tert-butyl) 1-ethyl 3-bromo-5,6-dihydroimidazo[1,5-a]pyrazine-1,7(8H)-dicarboxylate (CAS: 2108354-93-2; 0.578 g, 1.54 mmol) and cyclopropylboronic acid (CAS: 411235-57-9, 0.398 g, 4.63 mmol) in dioxane (8.0 mL) and H₂O (1.0 mL) was added K₃PO₄ (0.984 g, 4.63 mmol) and Pd(dppf)Cl₂ (0.226 g, 0.309 mmol) at room temperature under nitrogen gas. The reaction was stirred at 90° C. for 16 h. The resulting solution was concentrated in vacuo to give a solid that was purified by column chromatography (silica gel, 0-95% EtOAc:PE) to provide C9 (0.400 g, 77.2% yield) as a yellow gum. (LC/MS) m/z (M+H)⁺=336.1. ¹H NMR (400 MHz, CD₃OD) δ 4.84 (s, 2H), 4.30 (q, 2H), 4.14-4.04 (m, 2H), 3.86 (t, 2H), 1.94-1.86 (m, 1H), 1.53-1.48 (m, 9H), 1.36 (t, 3H), 1.00-0.96 (m, 4H).

### Step 2. Preparation of ethyl 3-cyclopropyl-5,6,7,8-tetrahydroimidazo[1,5-a]pyrazine-1-carboxylate hydrochloride (P5)

To a solution of C9 (0.300 g, 0.894 mmol) in DCM (5.0 mL) was added 2M HCl in dioxane (0.261 g, 7.16 mmol) and reaction stirred at room temperature for 16 h. The resulting suspension was concentrated in vacuo to provide P5 (0.243 g, crude) as a white solid, used directly in the next step without further purification. ¹H NMR (400 MHz, (CD₃)₂SO) δ 10.17 (s, 1H), 4.50 (s, 2H), 4.36 (t, 2H), 4.25 (q, 2H), 3.62-3.57 (m, 2H), 2.13-2.03 (m, 1H), 1.30-1.23 (m, 3H), 1.04-0.98 (m, 2H), 0.97-0.91 (m, 2H).

### Preparation 6

### rac-Ethyl (R)-1,7-dimethyl-4,5,6,7-tetrahydro-1H-pyrazolo[4,3-c]pyridine-3-carboxylate hydrochloride (P6)

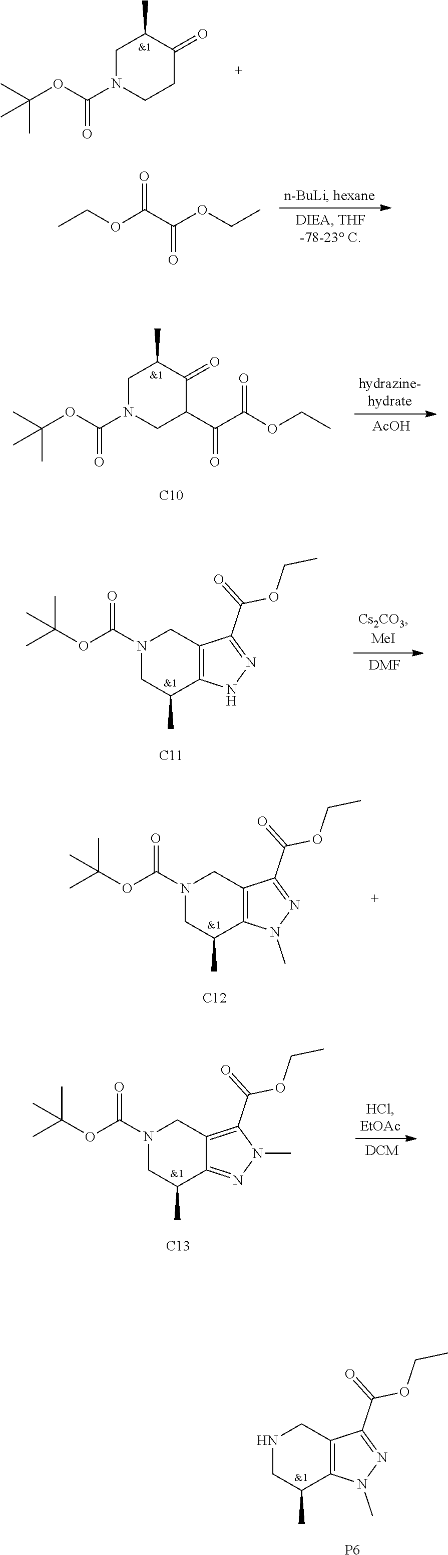

### Step 1: Preparation of rac-tert-butyl (5R)-3-(2-ethoxy-2-oxoacetyl)-5-methyl-4-oxopiperidine-1-carboxylate (C10)

A solution of diisopropylamine (4.2 g, 41 mmol) in THF (19.0 mL) was cooled to −78° C. before 2.5M n-BuLi in hexane (2.5 g, 39 mmol) was added. After the addition, the reaction mixture was warmed to 0° C. and stirred for 15 min. The reaction was then cooled to −78° C. and a solution of rac-tert-butyl (R)-3-methyl-4-oxopiperidine-1-carboxylate (CAS: 181269-69-2, 7.0 g, 33 mmol) in THF (81.0 mL) was slowly added while maintaining the temperature below −67° C. The mixture was stirred at −78° C. for about 1 h and 30 min before diethyl oxalate (4.8 g, 33 mmol) was added to the reaction solution. The resulting mixture was allowed to warm to room temperature and then stirred for 2 h. The mixture was neutralized with 1N HCl (80 mL) and extracted with EtOAc (2×200 mL). The combined organic layers were washed with brine (400 mL), dried over Na₂SO₄ and concentrated in vacuo. The residue was purified by column chromatography (silica gel, 0-10% EtOAc:PE) to provide C10 (4.8 g, 47% yield) as a yellow oil. (LC/MS) m/z (M+H)⁺=314.4. ¹H NMR (400 MHz, CDCl₃) δ 4.51-4.30 (m, 3H), 3.91-3.65 (m, 1H), 3.34-3.12 (m, 1H), 2.76-2.60 (m, 1H), 1.51-1.44 (m, 9H), 1.41-1.34 (m, 3H), 1.28-1.20 (m, 3H).

### Step 2: Preparation of rac-5-(tert-butyl) 3-ethyl (R)-7-methyl-1,4,6,7-tetrahydro-5H-pyrazolo[4,3-c]pyridine-3,5-dicarboxylate (C11)

To a solution of C10 (3.55 g, 11.3 mmol) in AcOH (14.2 mL), hydrazine hydrate was added (2.09 g, 65% wt, 27.2 mmol). During the addition, the reaction mixture became exothermic to 65° C. The reaction mixture was stirred for 1 h then concentrated in vacuo. The residue was diluted with H₂O followed by saturated aqueous NaHCO₃ (200 mL). The mixture was extracted with EtOAc (2×200 mL). The combined organic layers were dried with Na₂SO₄, filtered, concentrated in vacuo, and dried further under a high vacuum to provide C11 (3.45 g, crude) as a pale-yellow solid, used directly in the next step without further purification. (LC/MS) m/z (M+H)⁺=310.40.

### Step 3: Preparation of rac-5-(tert-butyl) 3-ethyl (R)-1,7-dimethyl-1,4,6,7-tetrahydro-5H-pyrazolo[4,3-c]pyridine-3,5-dicarboxylate (C12) and rac-5-(tert-butyl) 3-ethyl (R)-2,7-dimethyl-2,4,6,7-tetrahydro-5H-pyrazolo[4,3-c]pyridine-3,5-dicarboxylate (C13)

To a solution of C11 (3.45 g, 11.2 mmol) in DMF (28.6 mL), Cs₂CO₃ (3.65 g, 11.2 mmol) was added, followed by Mel (4.80 g, 33.5 mmol). The reaction mixture was stirred for 16 h at room temperature, diluted with H₂O (100 mL) and extracted with EtOAc (3×50 mL). The combined organic layer was washed with saturated aqueous NH₄Cl (2×100 mL) and H₂O (2×100 mL). The organic layer was dried over Na₂SO₄, filtered, and then concentrated in vacuo. The residue was purified by column chromatography (silica gel, 10-100% EtOAc:heptane) to provide C13 (2.12 g, 58.9% yield) and C12 (0.999 g, 27.7% yield).

C13: (LC/MS) m/z (M+H)⁺=324.1. ¹H NMR (400 MHz, CDCl₃) δ 4.74-4.41 (m, 2H), 4.38-4.27 (m, 2H), 4.17-4.10 (m, 3H), 3.98-3.65 (m, 1H), 3.36-2.88 (m, 2H), 1.48 (s, 9H), 1.38 (t, 3H), 1.26 (d, 3H).

C12: (LC/MS) m/z (M+H)⁺=324.1. ¹H NMR (400 MHz, CDCl₃) δ 5.18-4.87 (m, 1H), 4.43-4.30 (m, 2H), 4.27-3.99 (m, 2H), 3.85 (s, 3H), 3.23-2.87 (m, 2H), 1.51-1.43 (m, 9H), 1.41-1.33 (m, 3H), 1.24 (d, 3H).

### Step 4: Preparation of rac-ethyl (R)-1,7-dimethyl-4,5,6,7-tetrahydro-1H-pyrazolo[4,3-c]pyridine-3-carboxylate hydrochloride (P6)

To a solution of C12 (0.536 g, 1.66 mmol) in DCM (9.7 mL) was added 1M HCl in EtOAc (5.0 mL) at room temperature. The reaction was stirred at room temperature for 16 h. An additional portion of 1M HCl in EtOAc (5.0 mL) was added at room temperature then stirred for 1 h. The reaction was concentrated in vacuo to provide P6 (0.426 g, 99.1% yield) as an off-white solid. This was used in the next step without further purification. (LC/MS) m/z (M+H)⁺=224.2. ¹H NMR (400 MHz, CDCl₃) δ 10.58-10.31 (m, 1H), 10.18-9.87 (m, 1H), 4.58-4.49 (m, 1H), 4.46-4.32 (m, 3H), 3.92 (s, 3H), 3.51-3.25 (m, 3H), 1.57-1.53 (m, 3H), 1.40 (t, 3H).

### Preparation 7

### rac-Methyl (R)-3,5-dimethyl-5,6,7,8-tetrahydroimidazo[1,5-a]pyrazine-1-carboxylate hydrochloride (P7)

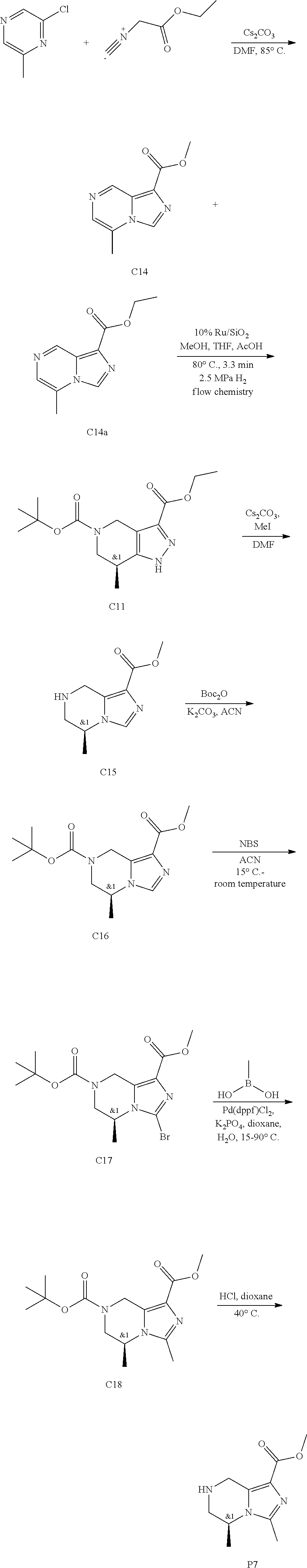

### Step 1. Preparation of methyl 5-methylimidazo[1,5-a]pyrazine-1-carboxylate (C14) and ethyl 5-methylimidazo[1,5-a]pyrazine-1-carboxylate (C14a)

To a mixture of 2-chloro-6-methylpyrazine (CAS: 38557-71-0, 15.0 g, 117 mmol) in DMF (150.0 mL) was added Cs₂CO₃ (57.0 g, 175 mmol) and ethyl 2-isocyanoacetate (CAS: 2999-46-4, 26.6 g, 233 mmol). The reaction was heated to 85° C. and stirred for 16 h. The light brown reaction mixture was filtered then the solid was washed with MeOH. The filtrate was concentrated in vacuo. The brown residue was purified by column chromatography (silica gel, 0-100% EtOAC:PE then 2% MeOH:EtOAc). Transesterification occurred during workup and purification to generate C14 (9.50 g, 42.6% yield) as a light brown solid (LC/MS) m/z (M+H)⁺=192.1 and a mixture of (2:1, C14a:C14) (1.1 g, 4.59% yield) (LC/MS) m/z (M+H)⁺=206.1. C14 was brought forward to the next step.

C14: ¹H NMR (400 MHz, (CD₃)₂SO) δ 9.32 (s, 1H), 8.68 (s, 1H), 7.74 (s, 1H), 3.90 (s, 3H), 2.63 (s, 3H).

### Step 2. Preparation of rac-methyl (R)-5-methyl-5,6,7,8-tetrahydroimidazo[1,5-a]pyrazine-1-carboxylate (C15)

The reaction was carried out via flow chemistry. C14 (2.90 g, 15.2 mmol) was suspended in MeOH (50.0 mL), THF (50.0 mL) and AcOH (0.6 mL) then heated to 50° C. to dissolve to a solution. The solution was pumped with a flow rate of 0.3 mL/min and the H₂ had a rate of 30 mL/min. The solution passed through the fixed bed (6.350 (¼″) mm) packed with granular catalyst 10% Ru/SiO₂ (15.3 g, 1.52 mmol) with a 5.0 mL volume and was hydrogenated for 3.3 min at 80° C. via flow at 2.5 MPa H₂. The light-yellow reaction was concentrated in vacuo then dried in high vacuum to provide C15 (2.96 g, crude) as a light brown oil. This was used in the next step without further purification. ¹H NMR (400 MHz, CDCl₃) δ 7.60 (s, 1H), 4.43 (d, 1H), 4.27-4.15 (m, 2H), 3.87 (s, 3H), 3.32 (dd, 1H), 2.84 (dd, 1H), 2.68 (s, 1H), 1.53 (d, 3H).

### Step 3. Preparation of rac-7-(tert-butyl) 1-methyl (R)-5-methyl-5,6-dihydroimidazo[1,5-a]pyrazine-1,7(8H)-dicarboxylate (C16)

To a solution of C15 (2.96 g, 15.2 mmol) in ACN (60.0 mL) was added Boc₂O (4.97 g, 22.8 mmol) and K₂CO₃ (6.29 g, 45.5 mmol) at room temperature. The reaction was stirred at room temperature for 16 h. The light brown reaction mixture was filtered then concentrated in vacuo. The brown residue was purified by column chromatography (silica gel; 0-100% EtOAc:PE) to provide C16 (3.02 g, 67.5% yield) as an off-white solid. (LC/MS) m/z (M+H)⁺=296.1. ¹H NMR (400 MHz, CDCl₃) δ 7.53 (s, 1H), 5.01-4.77 (m, 2H), 4.33-4.21 (m, 1H), 3.89 (s, 3H), 3.55-3.34 (m, 2H), 1.53-1.49 (m, 12H).

### Step 4. Preparation of rac-7-(tert-butyl) 1-methyl (R)-3-bromo-5-methyl-5,6-dihydroimidazo[1,5-a]pyrazine-1,7(8H)-dicarboxylate (C17)

To a solution of C16 (3.02 g, 10.2 mmol) in ACN (50.0 mL) was added NBS (2.73 g, 15.4 mmol) at 15° C. The reaction was stirred at room temperature for 16 h. The light brown reaction mixture was concentrated in vacuo. The brown residue was purified by column chromatography (silica gel, 0-100% EtOAc:PE) which gave an impure C17 as a light brown solid. The solid was triturated with EtOAc:PE (1:5). The solid was filtered then rinsed with EtOAc:PE (5:1) to provide C17 (1.32 g, 34.4% yield) as a white solid. (LC/MS) m/z (M+2H)⁺=376.0. ¹H NMR (400 MHz, CDCl₃) δ 5.62-5.23 (m, 1H), 4.54-4.22 (m, 3H), 3.93-3.86 (m, 3H), 3.48-3.16 (m, 1H), 1.51 (s, 9H), 1.43 (d, 3H).

### Step 5. Preparation of rac-7-(tert-butyl) 1-methyl (R)-3,5-dimethyl-5,6-dihydroimidazo[1,5-a]pyrazine-1,7(8H)-dicarboxylate (C18)

To a solution of C17 (1.30 g, 3.47 mmol) and methylboronic acid (0.624 g, 10.4 mmol) in dioxane (28.0 mL) and H₂O (7.0 mL) was added K₃PO₄ (2.21 g, 10.4 mmol) then Pd(dppf)Cl₂ (0.519 g, 0.695 mmol) at 15° C. under nitrogen. The reaction was heated to 90° C. for 16 h. The brown reaction mixture was extracted with EtOAc (30 mL). The organic phase was dried over Na₂SO₄ and concentrated in vacuo. The brown residue was purified by column chromatography (silica gel, 0-100% EtOAc:PE then 2% MeOH:EtOAc) then dried by high vacuum to provide C18 (0.460 g, 42.8%) as a light brown solid. (LC/MS) m/z (M+H)⁺=310.2. ¹H NMR (400 MHz, CDCl₃) δ 5.51-5.15 (m, 1H), 4.52-4.14 (m, 3H), 3.87 (s, 3H), 3.32-3.10 (m, 1H), 2.39 (s, 3H), 1.50 (s, 9H), 1.36 (d, 3H).

### Step 6. Preparation of rac-methyl (R)-3,5-dimethyl-5,6,7,8-tetrahydroimidazo[1,5-a]pyrazine-1-carboxylate hydrochloride (P7)

To a solution of C18 (140 mg, 0.452 mmol) in DCM (4.0 mL) was added 2M HCl in dioxane (8.0 mL) at room temperature. The reaction was heated to 40° C. and stirred for 2 to 5 h. The light brown reaction mixture was concentrated in vacuo and dried further under high vacuum to provide P7 (0.111 g, crude) as a light brown solid. The solid was used in the next step without further purification.

### Preparation 8

### (S)-5-(tert-Butoxycarbonyl)-7-methyl-4,5,6,7-tetrahydro-1H-pyrazolo[4,3-c]pyridine-3-carboxylic acid (P8)

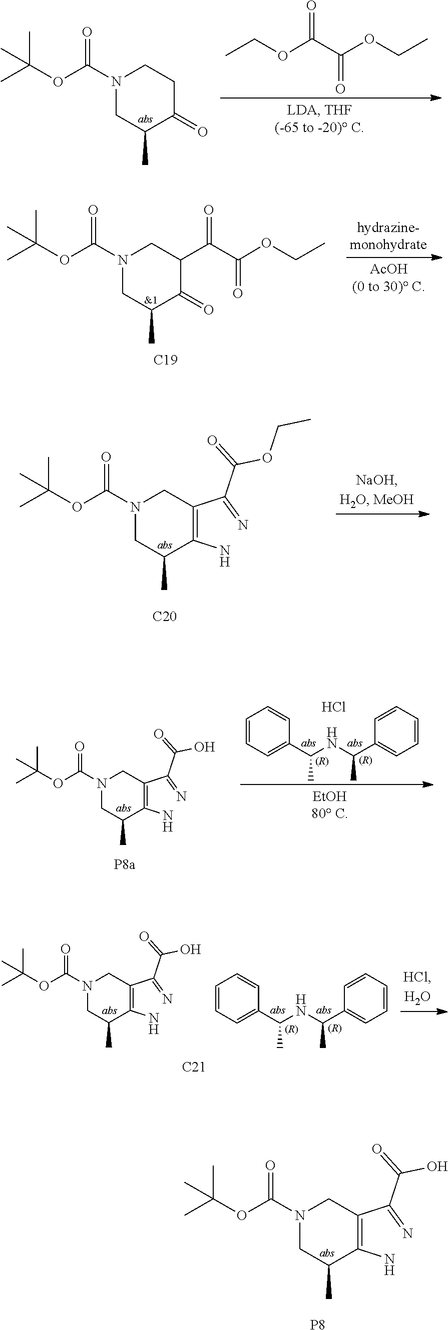

### Step 1. Preparation of rac-tert-butyl (5S)-3-(2-ethoxy-2-oxoacetyl)-5-methyl-4-oxopiperidine-1-carboxylate (C19)

Under a nitrogen balloon at −65° C., LDA (44.4 g, 0.414 mol, 207 mL) was added to THF (350 mL) then a solution of tert-butyl (S)-3-methyl-4-oxopiperidine-1-carboxylate (CAS: 2092486-33-2; 88.4 g, 0.414 mol) in THF (350 mL) was added dropwise over 1 h to keep the inner temperature below −65° C. The reaction mixture was stirred at −65° C. for 1 h before a solution of diethyl oxalate (CAS: 95-92-1; 60.6 g, 0.414 mol) in THF (350 mL) was added dropwise at −65° C. After the addition, the reaction mixture was stirred at −65° C. for 1 h, then warmed to −20° C. and stirred for 40 min. At 0° C. under nitrogen gas, the reaction mixture was quenched with 1M aqueous KHSO₄ solution (1415 mL), which caused a white precipitate to form. The suspension was filtered, and then the filtrate was extracted with EtOAc (3×800 mL). The combined organic layer was washed with brine (800 mL), dried over Na₂SO₄, filtered then concentrated in vacuo to provide C19 (129 g, crude) as a yellow oil. The oil was used directly in the next step without further purification. (LCMS) m/z (M-tert-butyl group)⁺=257.9. ¹H NMR (400 MHz, (CD₃)₂SO) δ 12.21 (br s, 1H), 4.47-4.17 (m, 3H), 4.06-3.47 (m, 2H), 3.29-3.17 (m, 1H), 2.59-2.53 (m, 1H), 1.45-1.37 (m, 9H), 1.28-1.21 (m, 3H), 1.18-0.95 (m, 3H).

### Step 2. Preparation of enriched 5-(tert-butyl) 3-ethyl (S)-7-methyl-1,4,6,7-tetrahydro-5H-pyrazolo[4,3-c]pyridine-3,5-dicarboxylate (C20)

At 0° C., to a suspension of C19 (139 g, 0.443 mol) in AcOH (552 mL) was added hydrazine monohydrate (62.7 g, 1.06 mol) which caused the internal temperature to increase to −30° C. The reaction mixture was stirred at room temperature under nitrogen gas for 1 h then was diluted with H₂O (500 mL) and saturated NaHCO₃ (1000 mL). The diluted suspension was extracted with EtOAc (2×800 mL). The combined organic layer was dried over Na₂SO₄, filtered then concentrated in vacuo to give a yellow gum. The gum was purified by column chromatography (silica gel, (1:1) EtOAc:PE) to give the enriched S-enantiomer C20 (123 g, 89.7% yield) as a yellow gum. (LCMS) m/z (M+H)⁺=310.0. ¹H NMR (400 MHz, (CD₃)₂SO) δ 13.71-13.28 (m, 1H), 4.63-4.18 (m, 4H), 3.86-3.59 (m, 1H), 3.12-2.80 (m, 2H), 1.40-1.38 (m, 9H), 1.30-1.24 (m, 3H), 1.17-1.13 (m, 3H). The chiral purity was determined using SFC analytical method on Chiralpak AD-3 100 mm×4.6 mm×3 μm column; Mobile phase A: CO₂/Mobile phase B: EtOH with 0.2% of NH₃; gradient: 5-40% of Mobile Phase B over 3 min then held at 5% of Mobile phase B for 1 min; backpressure: 1500 psi; flow rate: 2.8 mL/min; column temperature: 35° C. to provide C20: major peak ((SFC-MS) m/z (M-tert-butyl group)⁺=254.04 at retention time: 2.218 min, 94% ee).

### Step 3. Preparation of enriched (S)-5-(tert-butoxycarbonyl)-7-methyl-4,5,6,7-tetrahydro-1H-pyrazolo[4,3-c]pyridine-3-carboxylic acid (P8a)

To a solution of C20 (124 g, 0.400 mol) in MeOH (452 mL) and H₂O (452 mL) was added NaOH (32.0 g, 0.800 mol) then stirred at room temperature for 24 h. The reaction mixture was diluted with H₂O (200 mL) then cooled to 0° C. before the diluted reaction was acidified to pH˜3 with 1M aqueous HCl solution. The acidic reaction mixture was filtered then the filter cake was collected and lyophilized to give the enriched S-enantiomer P8a (88.6. g, crude) as a white solid. The solid was used directly in the next step without further purification. (LCMS) m/z (M+H)⁺=282.0. ¹H NMR (400 MHz, (CD₃)₂SO) δ 13.22 (br s, 1H), 4.66-4.34 (m, 2H), 3.86-3.60 (m, 1H), 3.12-2.82 (m, 2H), 1.41 (s, 9H), 1.17 (d, 3H). The chiral purity was determined using SFC analytical method on Chiralpak IG-3 100 mm×4.6 mm×3 μm column; Mobile phase A: CO₂/Mobile phase B: 50% MeOH with 0.2% of NH₃; backpressure: 1500 psi; flow rate: 2.8 mL/min; column temperature: 35° C. to provide P8a: major peak ((SFC-MS) m/z (M-tert-butyl group)⁺=226.03 at retention time: 0.784 min, 96% ee)

### Step 4. Preparation of (S)-5-(tert-butoxycarbonyl)-7-methyl-4,5,6,7-tetrahydro-1H-pyrazolo[4,3-c]pyridine-3-carboxylic acid (+)-bis[(R)-1-phenylethyl]amine salt (C21)

The reaction was conducted in six batches in parallel then combined. To a mixture of P8a (0.180 kg, 0.640 mol) in EtOH (3.4 L) was added (+)-bis[(R)-1-phenylethyl]amine (CAS: 23294-41-9; 144 g, 0.640 mol) in one portion at room temperature to form the first batch. The reaction mixture of the first batch was stirred at room temperature for 10 min then white precipitate formed. The suspension of the first batch was stirred at 80° C. for 1 h to form a clear solution then stirred at room temperature for 20 h. The white suspension of the first batch was filtered then the filter cake was washed with EtOH (3×300 mL).

The filter cake from the first batch was collected then combined with the filter cakes from the additional five batches of the same reaction utilizing P8a (0.180 kg, 0.640 mol). The combined filter cake was dried in an oven at 50° C. for 18 h to provide C21 (1.42 kg, crude) as a white solid. The solid was used directly in the next step without further purification. (LCMS) m/z (M+H)⁺=282.1. ¹H NMR (400 MHz, CD₃OD) δ 7.46-7.36 (m, 6H), 7.29-7.23 (m, 4H), 4.76-4.53 (m, 2H), 3.95-3.86 (m, 2H), 3.83-3.70 (m, 1H), 3.25-3.18 (m, 1H), 2.99-2.89 (m, 1H), 1.52 (dd, 6H), 1.48 (s, 9H), 1.25 (d, 3H). The chiral purity was determined using SFC analytical method on Chiralpak IG 50 mm×4.6 mm×3 μm column; Mobile phase A: CO₂/Mobile phase B: MeOH with 0.05% of DEA; gradient: 20-40% of Mobile phase B over 1.5 min and hold 40% of Mobile phase B for 1.0 min then 20% of Mobile Phase B for 0.5 min; flow rate of 4.0 mL/min; backpressure: 1500 psi; column temperature: 35° C. to provide C21: peak 1 (retention time: 1.019 min, 100% ee).

### Step 5. Preparation of (S)-5-(tert-butoxycarbonyl)-7-methyl-4,5,6,7-tetrahydro-1H-pyrazolo[4,3-c]pyridine-3-carboxylic acid (P8)

To a suspension of C21 (48.2 g, 95.1 mmol) in H₂O (482 mL) was added 1M aqueous HCl solution (99 mL) to pH˜1. The reaction mixture was stirred at room temperature for 1 h then filtered. The filter cake was collected then lyophilized to give P8 (24.3 g, crude) as a white solid. The solid was used directly in the next step without further purification. (LCMS) m/z (M+H)⁺=282.0. ¹H NMR (400 MHz, (CD₃)₂SO) δ 13.15 (br s, 1H), 4.63-4.47 (m, 1H), 4.44-4.33 (m, 1H), 3.84-3.61 (m, 1H), 3.19-3.00 (m, 1H), 2.94-2.83 (m, 1H), 1.41 (s, 9H), 1.17 (d, 3H). The chiral purity was determined using SFC analytical method on Chiralpak IG-3 100 mm×4.6 mm×3 μm column; Mobile phase A: 50% of CO₂/Mobile phase B: 50% of MeOH with 0.2% NH₃; backpressure: 1500 psi; flow rate: 2.8 mL/min; column temperature: 35° C. to provide P8: peak 1 ((SFC-MS) m/z (M-tert-butyl group)⁺=226.00 at retention time: 0.757 min, 99% ee). [α]²⁸_{D}=+11.351 (c=1.5 (g/L in MeOH).

### Preparation 9

### (S)-1-Isopropyl-7-methyl-4,5,6,7-tetrahydro-1H-pyrazolo[4,3-c]pyridine-3-carboxylic acid

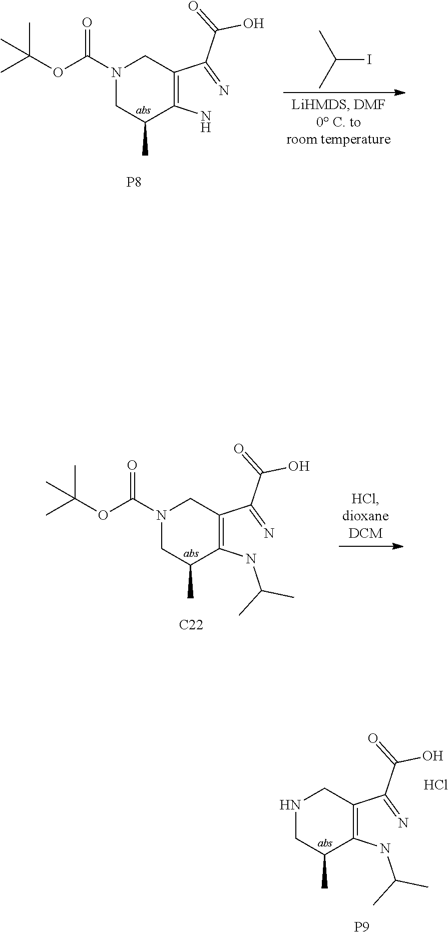

### Step 1. Preparation of (S)-5-(tert-butoxycarbonyl)-1-isopropyl-7-methyl-4,5,6,7-tetrahydro-1H-pyrazolo[4,3-c]pyridine-3-carboxylic acid (C22)

At 0° C. under nitrogen gas, to a solution of P8 (12.4 g, 44.1 mmol) in DMF (220 mL) was added 1M LiHMDS (132 mL). The reaction mixture was warmed to room temperature and stirred for 2 h before cooling the reaction mixture again to 0° C. To the cooled reaction mixture was added 2-iodopropane (11.2 g, 66.1 mmol) then the suspension was warmed to room temperature and stirred for 48 h. The reaction mixture was cooled to 0° C. and another portion of 2-iodopropane (1.12 g, 6.61 mmol) was added. The suspension was warmed to room temperature and stirred for 48 h. The reaction mixture was cooled to 0° C. then quenched with H₂O (500 mL). The aqueous phase was washed with EtOAc (2×200 mL) then the combined organic layer was discarded. The aqueous layer was acidified with 1M aqueous HCl to pH˜3 then extracted with EtOAc (2×200 mL). The combined organic layer was washed with brine (2×300 mL), dried over Na₂SO₄ and concentrated in vacuo to give C22 (13.8 g, 96.8% yield) as a yellow solid. (LCMS) m/z (M+H)⁺=324.0. ¹H NMR (400 MHz, (CD₃)₂SO) δ 12.60 (br s, 1H), 4.99-4.73 (m, 1H), 4.57-4.41 (m, 1H), 4.19-3.86 (m, 2H), 3.17-3.00 (m, 2H), 1.42-1.36 (m, 15H), 1.17-1.08 (m, 3H). The chiral purity was determined using SFC analytical method on (S,S) Whelk-01, 150 mm×4.6 mm×3.5 μm column; Mobile phase A: CO₂/Mobile phase B: MeOH (0.2% isopropylamine); gradient: 10-50% of Mobile phase B in 6.00 min then held at 10% of Mobile phase B for 2.00 min; backpressure: 2000 psi; flow rate: 1.5 mL/min; column temperature: 35° C. to provide C22: peak 1 (retention time: 2.352 min, 100% ee). [α]³⁴_{D}=−100.10 (c=5.4 (g/L) in ACN).

### Step 2. Preparation of (S)-1-isopropyl-7-methyl-4,5,6,7-tetrahydro-1H-pyrazolo[4,3-c]pyridine-3-carboxylic acid hydrochloride (P9)

To a solution of C22 (12.7 g, 39.3 mmol) in DCM (40 mL) was added 2M HCl in dioxane (500 mL). The reaction mixture was stirred at room temperature for 5 h then was concentrated in vacuo to provide P9 (9.40 g, crude) as a white solid. The solid was used directly in the next step without further purification. (LCMS) m/z (M+H)⁺=224.2. ¹H NMR (400 MHz, CD₃OD) δ 4.68-4.58 (m, 1H), 4.45 (d, 1H), 4.31 (d, 1H), 3.62-3.38 (m, 3H), 1.54 (d, 3H), 1.48 (d, 3H), 1.44 (d, 3H). The chiral purity was determined using SFC analytical method on Chiralcel OX-3 100 mm×4.6 mm×3 μm column; Mobile phase A: CO₂/Mobile phase B: MeOH containing 0.05% of DEA; gradient: 5-40% of Mobile Phase B in 3.0 min then held at 40% of Mobile phase B for 0.9 min then held at 5% of Mobile phase B for 0.1 min; backpressure: 100 bar; flow rate: 2.8 mL/min; column temperature: 40° C. to provide P9: peak 1 (retention time: 3.037 min, 100% ee). [α]³³_{D}=−7.212 (c=1.5 (g/L) in MeOH).

### Preparation 10

### (S)-1-Ethyl-7-methyl-4,5,6,7-tetrahydro-1H-pyrazolo[4,3-c]pyridine-3-carboxylic acid hydrochloride (P10)

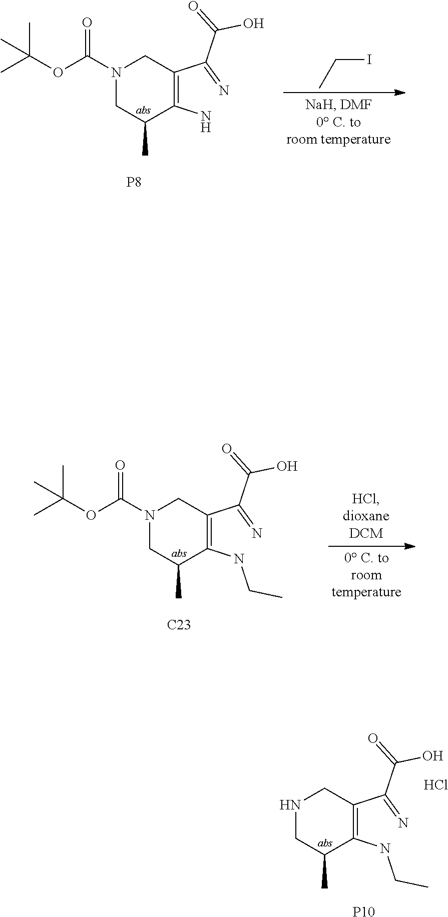

### Step 1. Preparation of (S)-5-(tert-butoxycarbonyl)-1-ethyl-7-methyl-4,5,6,7-tetrahydro-1H-pyrazolo[4,3-c]pyridine-3-carboxylic acid (C23)

At 0° C. under nitrogen gas, to a solution of P8 (10.0 g, 35.5 mmol) in DMF (190 mL) was added NaH (4.27 g, 107 mmol). The reaction mixture was stirred 0° C. for 30 min then a solution of iodoethane (6.65 g, 42.7 mmol) in DMF (10 mL) was added. The suspension was warmed to room temperature and stirred for 3 h then quenched with H₂O (150 mL). The aqueous phase was extracted with EtOAc (2×150 mL) then the combined organic layer was discarded. The aqueous phase was acidified with 1M aqueous HCl to pH˜3 then extracted again with EtOAc (150 mL×3). The combined organic layer was washed with brine (2×100 mL), dried over Na₂SO₄ and concentrated in vacuo to give a yellow solid. The solid was suspended in (1:10) EtOAc:PE (25 mL) and stirred at room temperature for 30 min. The suspension was filtered, and the filter cake was collected to provide C23 (8.75 g, crude) as a white solid. The solid was used directly in the next step without further purification. (LCMS) m/z (M+H)⁺=310.0. ¹H NMR (400 MHz, (CD₃)₂SO) δ 12.65 (br s, 1H), 4.85 (dd, 1H), 4.21-3.84 (m, 4H), 3.22-2.98 (m, 2H), 1.41 (br s, 9H), 1.35 (t, 3H), 1.16-1.08 (m, 3H). The chiral purity was determined using Chiral HPLC analytical method on Chiralcel OD-RH 150 mm×4.6 mm×5 μm column; Mobile phase A: H₂O (1.5 mL TFA)/Mobile phase B: ACN (1.5 mL TFA); gradient: 10-80% of Mobile phase B over 8 min then 10% of Mobile phase B in 1 min then a hold at 10% of Mobile phase B for 6 min, flow rate: 0.8 mL/min; column temperature: 30° C. to provide C23: peak 1 (retention time: 9.514 min, 100% ee). [α]³⁴_{D}=−95.69 (c=0.4 (g/L) in ACN).

### Step 2. Preparation of (S)-1-ethyl-7-methyl-4,5,6,7-tetrahydro-1H-pyrazolo[4,3-c]pyridine-3-carboxylic acid hydrochloride (P10)

At 0° C., to C23 (8.00 g, 25.9 mmol) was added 2M HCl in dioxane (160 mL). The reaction mixture was stirred at room temperature for 4 h then was concentrated in vacuo to provide a yellow solid. The solid was dissolved in 2M HCl in dioxane (40 mL) and stirred at room temperature for 2 h then was concentrated in vacuo to provide P10 (7.10 g, crude) as a yellow solid. The solid was used directly in the next step without further purification. (LCMS) m/z (M+H)⁺=210.2. ¹H NMR (400 MHz, (CD₃)₂SO) δ 12.97 (br s, 1H), 10.05 (s, 1H), 9.49 (s, 1H), 4.23-4.03 (m, 4H), 3.41-3.30 (m, 2H), 3.26-3.18 (m, 1H), 1.39-1.32 (m, 6H). The chiral purity was determined using SFC analytical method on Chiralcel OX-3 100 mm×4.6 mm, 3 μm column; Mobile phase A: CO₂/Mobile phase B: MeOH with 0.05% of DEA; Isocratic: 40% of Mobile phase B; backpressure: 100 bar; flow rate: 2.8 mL/min; column temperature: 40° C. to provide P10: peak 1 (retention time: 1.631 min, 100% ee). [α]³³_{D}=−1.584 (c=1.5 (g/L) in MeOH).

### Preparation 11

### 3-(4-(Aminomethyl)phenyl)-1,1-dimethylurea hydrochloride (P11)

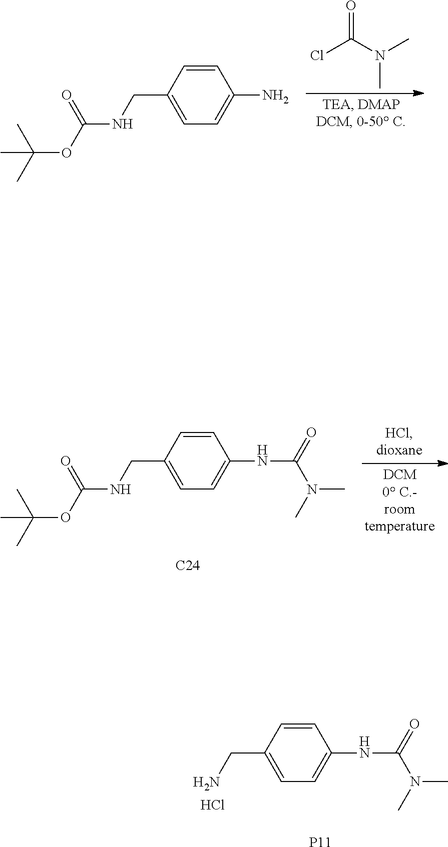

### Step 1. Preparation of tert-butyl (4-(3,3-dimethylureido)benzyl)carbamate (C24)

At 0° C. under nitrogen gas, to a solution of tert-butyl (4-aminobenzyl)carbamate (CAS: 94838-55-8; 15.0 g, 67.5 mmol) in DCM (150 mL), TEA (20.5 g, 202 mmol) and DMAP (0.824 g, 6.75 mmol) was added dimethylcarbamic chloride (14.5 g, 135 mmol) dropwise. The reaction mixture was stirred at 50° C. for 16 h then was diluted with MeOH (150 mL) and concentrated in vacuo to give a residue. The residue was suspended in H₂O (120 mL) then rapidly stirred at room temperature for 1 h. The suspension was filtered then the filter cake was collected and triturated with MeOH:EtOAc:MTBE (5 mL: 25 mL: 250 mL) at room temperature for 1 h. The suspension was filtered, and the filter cake was collected then concentrated in vacuo to provide C24 (14.6 g, 73.8% yield) as a white solid. (LC/MS) m/z (M+H)⁺=294.1. ¹H NMR (400 MHz, (CD₃)₂SO) δ 8.21 (s, 1H), 7.37 (d, 2H), 7.30 (t, 1H), 7.08 (d, 2H), 4.03 (d, 2H), 2.91 (s, 6H), 1.39 (s, 9H)

### Step 2. Preparation of 3-(4-(aminomethyl)phenyl)-1,1-dimethylurea hydrochloride (P11)

At 0° C., to a stirred suspension of C24 (23 g, 71 mmol) in DCM (130 mL) was added 2M HCl in dioxane (270 mL) dropwise. The reaction mixture was stirred at room temperature for 3 h then was concentrated in vacuo to provide P11 (15 g, 83.4%) as a yellow solid. The solid was used directly in the next step without additional purification. (LC/MS) m/z (2M+H)⁺=387.1. ¹H NMR (400 MHz, (CD₃)₂SO) δ 8.40 (s, 1H), 8.25 (br s, 2H), 7.53-7.47 (m, 2H), 7.35-7.29 (m, 2H), 3.91 (q, 2H), 2.92 (s, 6H).

### Preparation 12

### N-(4-(Aminomethyl)phenyl)-4-methylpiperazine-1-carboxamide hydrochloride (P12)

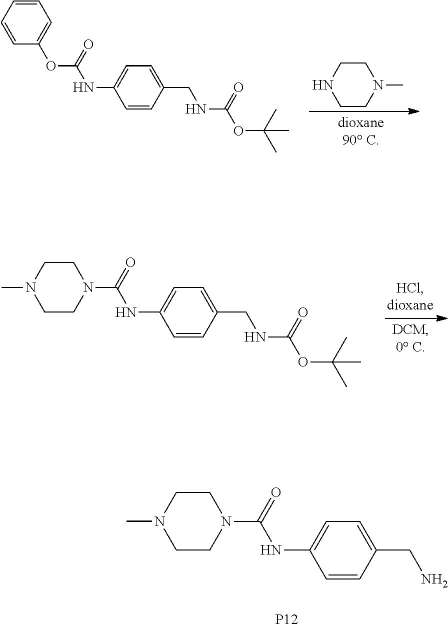

### Step 1. Preparation of tert-butyl (4-(4-methylpiperazine-1-carboxamido)benzyl)carbamate (C25)

To a solution of phenyl (4-(((tert-butoxycarbonyl)amino)methyl)phenyl)carbamate (CAS: 1632297-04-2; 8.0 g, 23 mmol) in dioxane (120 mL) was added 1-methylpiperazine (CAS: 109-01-3; 2.8 g, 28 mmol). The reaction mixture was stirred at 90° C. overnight. The suspension was diluted with EtOAc (100 mL) and washed with 1M NaOH (2×50 mL) then brine (50 mL). The organic layer was dried with Na₂SO₄ and concentrated in vacuo. The residue was triturated with PE (2×50 mL) over 20 min then the suspension was filtered. The filter cake was collected to provide C25 (7.7 g, crude) as a brown solid. The solid was used directly in the next step without further purification. (LC/MS) m/z (M+H)⁺=349.2. ¹H NMR (400 MHz, (CD₃)₂SO) δ 8.44 (s, 1H), 7.35 (d, 2H), 7.28 (t, 1H), 7.07 (d, 2H), 4.05-3.95 (m, 2H), 3.40 (t, 4H), 2.29 (t, 4H), 2.18 (s, 3H), 1.40-1.32 (m, 9H).

### Step 2. Preparation of N-(4-(aminomethyl)phenyl)-4-methylpiperazine-1-carboxamide hydrochloride (P12)

To a stirred solution of C25 (7.7 g, 22 mmol) in DCM (20 mL) was added HCl in dioxane (0.050 g, 1.4 mmol). The reaction mixture was stirred at 0° C. for 4 h then concentrated in vacuo and lyophilized to provide P12 (6.9 g, crude) as a white solid. The solid was used directly in the next step without further purification. (LC/MS) m/z (M+H)⁺=249.1. ¹H NMR (400 MHz, (CD₃)₂SO) δ 11.27 (br s, 1H), 9.08 (s, 1H), 8.36 (br s, 3H), 7.53-7.47 (m, 2H), 7.34-7.29 (m, 2H), 4.25 (d, 2H), 3.87 (q, 2H), 3.31-3.18 (m, 2H), 3.04-2.89 (m, 2H), 2.72 (s, 3H).

### Preparation 13

### Ethyl (S)-1-isopropyl-7-methyl-4,5,6,7-tetrahydro-1H-pyrazolo[4,3-c]pyridine-3-carboxylate hydrochloride (P13)

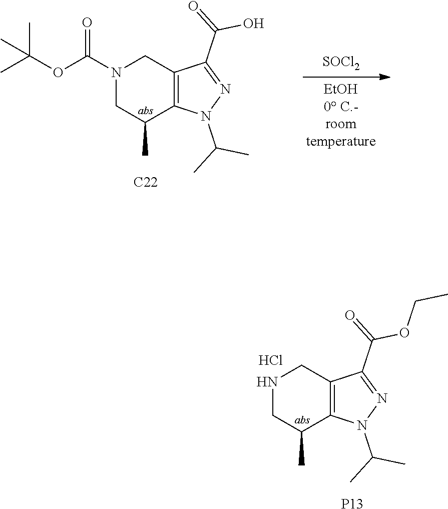

### Step 1. Preparation of ethyl (S)-1-isopropyl-7-methyl-4,5,6,7-tetrahydro-1H-pyrazolo[4,3-c]pyridine-3-carboxylate hydrochloride (P13)

To a stirred solution of C22 (0.500 g, 1.55 mmol) in EtOH (3 mL) was added SOCl₂ (1.29 g, 10.8 mmol) dropwise at 0° C. The reaction mixture was stirred at room temperature for 66 h then concentrated in vacuo to provide P13 (0.426 g, 95.7% yield) as a light-yellow solid. (LC/MS) m/z (M+H)⁺=252.2.

### Preparation 14

### Ethyl 1-(1-methylcyclopropyl)-4,5,6,7-tetrahydro-1H-pyrazolo[4,3-c]pyridine-3-carboxylate (P14)

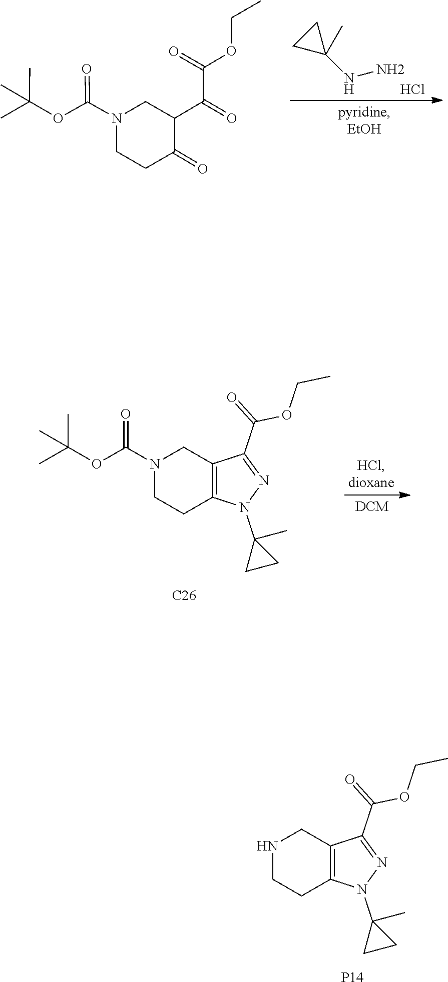

### Step 1. Preparation of 5-(tert-butyl) 3-ethyl 1-(1-methylcyclopropyl)-1,4,6,7-tetrahydro-5H-pyrazolo[4,3-c]pyridine-3,5-dicarboxylate (C26)

The same procedure was followed from Preparation 2, step 1 with tert-butyl 3-(2-ethoxy-2-oxoacetyl)-4-oxopiperidine-1-carboxylate (CAS: 518990-24-4; 0.577 g, 1.93 mmol) and (1-methylcyclopropyl)hydrazine hydrochloride (CAS: 2737246-42-1, 0.166 g, 1.93 mmol). The residue was purified by column chromatography (silica gel, 0-30% EtOAc:PE) to provide C26 (0.233 g, 34.6% yield) as a light-brown oil. (LC/MS) m/z (M+H)⁺=350.1. ¹H NMR (400 MHz, CDCl₃) δ 4.58 (s, 2H), 4.38 (q, 2H), 3.74-3.66 (m, 2H), 2.83-2.74 (m, 2H), 1.52 (s, 3H), 1.48 (s, 9H), 1.37 (t, 3H), 1.28-1.23 (m, 2H), 0.97-0.91 (m, 2H).

### Step 2. Preparation of ethyl 1-(1-methylcyclopropyl)-4,5,6,7-tetrahydro-1H-pyrazolo[4,3-c]pyridine-3-carboxylate (P14)

The same procedure was followed from Preparation 5, step 2 with C26 (0.233 g, 0.667 mmol) to provide P14 (0.191 g, crude) as a light-yellow solid, which was used directly in the next step without further purification. (LC/MS) m/z (M+H)⁺=250.1. ¹H NMR (400 MHz, CD₃OD) δ 4.39-4.33 (m, 2H), 3.66 (s, 2H), 3.56 (t, 2H), 3.19 (t, 2H), 1.55 (s, 3H), 1.38 (t, 3H), 1.31-1.26 (m, 2H), 1.09-1.04 (m, 2H).

### Preparation 15

### Ethyl 4,5,6,7-tetrahydrothieno[3,2-c]pyridine-3-carboxylate hydrochloride (P15)

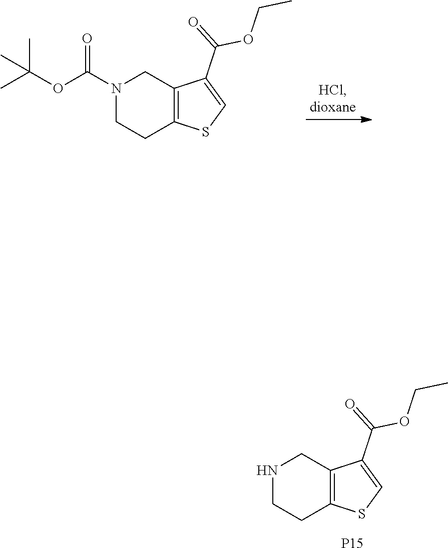

### Step 1. Preparation of ethyl 4,5,6,7-tetrahydrothieno[3,2-c]pyridine-3-carboxylate hydrochloride (P15)

The same reaction was conducted in two batches then combined. To 5-(tert-butyl) 3-ethyl 6,7-dihydrothieno[3,2-c]pyridine-3,5(4H)-dicarboxylate (CAS: 1363381-39-8; 50.0 mg, 0.161 mmol) was added HCl in dioxane (0.234 g, 6.42 mmol) to form the first batch. The solution of the first batch was stirred at room temperature for 2 h then concentrated in vacuo to provide a white solid.

A second batch of the same reaction was conducted with 5-(tert-butyl) 3-ethyl 6,7-dihydrothieno[3,2-c]pyridine-3,5(4H)-dicarboxylate (CAS: 1363381-39-8; 0.450 g, 1.45 mmol). The solids from the two batches were combined to provide P15 (0.339 g, crude) as a white solid. The solid was used directly in the next step without further purification. (LC/MS) m/z (M+H)⁺=212.0.

### Preparation 16

### Ethyl 1-methyl-5-(((trifluoromethyl)sulfonyl)oxy)-6,7-dihydro-1H-indazole-3-carboxylate (P16)

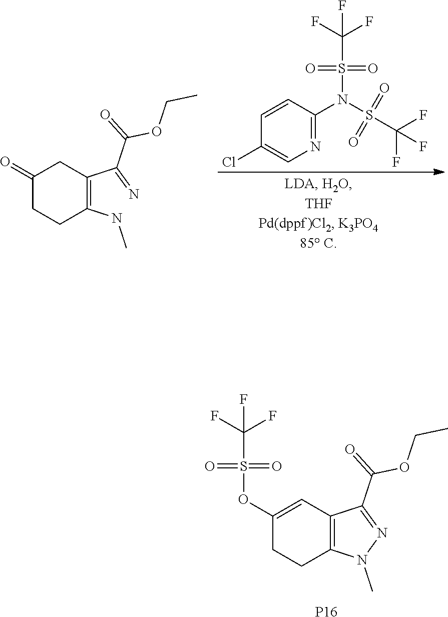

### Step 1. Preparation of ethyl 1-methyl-5-(((trifluoromethyl)sulfonyl)oxy)-6,7-dihydro-1H-indazole-3-carboxylate (P16)

To a suspension of ethyl 1-methyl-5-oxo-4,5,6,7-tetrahydro-1H-indazole-3-carboxylate (CAS: 2090404-84-3; 0.500 g, 1.41 mmol) and N-(5-chloropyridin-2-yl)-1,1,1-trifluoro-N-((trifluoromethyl)sulfonyl)methanesulfonamide (CAS: 145100-51-2; 0.409 g, 2.12 mmol) in H₂O (1 mL) and THF (10 mL) was added Pd(dppf)Cl₂ (0.103 g, 0.141 mmol) and K₃PO₄ (0.899 g, 4.23 mmol). The reaction mixture was degassed with nitrogen gas and stirred at 85° C. for 16 h then concentrated in vacuo. The residue was purified by column chromatography (silica gel, 0-38% THF:PE) to provide P16 (0.470 g, 94.2% yield) as a pink solid. (LC/MS) m/z (M+H)⁺=355.0

### Preparation 17

### Ethyl 1-(2,2,2-trifluoroethyl)-4,5,6,7-tetrahydro-1H-pyrazolo[4,3-c]pyridine-3-carboxylate

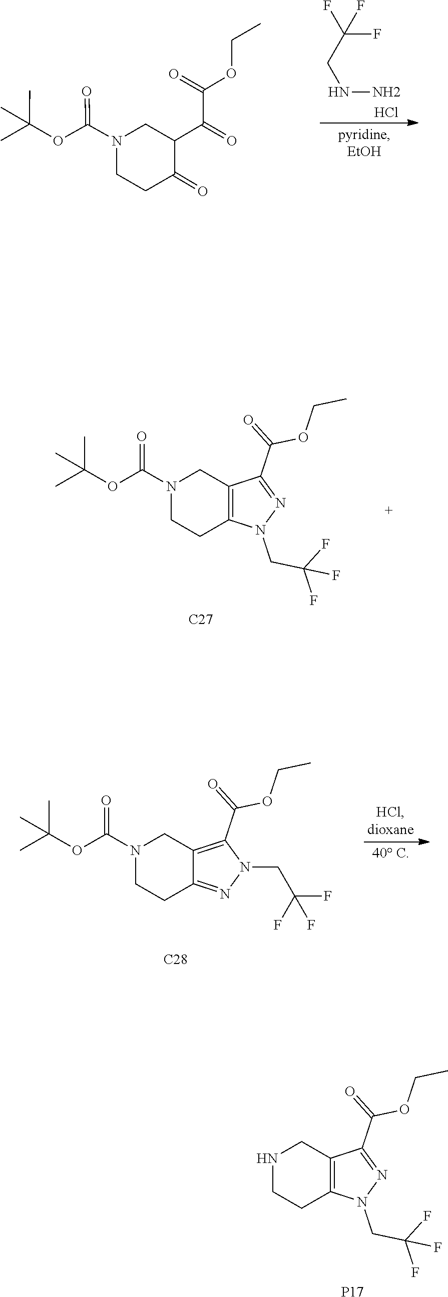

### Step 1. Preparation of 5-(tert-butyl) 3-ethyl 1-(2,2,2-trifluoroethyl)-1,4,6,7-tetrahydro-5H-pyrazolo[4,3-c]pyridine-3,5-dicarboxylate (C27) and 2-((3-(ethoxycarbonyl)-2-(2,2,2-trifluoroethyl)-4,5,6,7-tetrahydro-2H-pyrazolo[4,3-c]pyridine-5-carbonyl)oxy)-2-methylpropan-1-ylium (C28)

To a suspension of (2,2,2-trifluoroethyl)hydrazine hydrochloride (CAS: 1081515-82-3; 0.226 g, 1.50 mmol) in EtOH (5 mL) and pyridine (0.476 g, 6.01 mmol) was added tert-butyl 3-(2-ethoxy-2-oxoacetyl)-4-oxopiperidine-1-carboxylate (CAS: 518990-24-4; 0.450 g, 1.50 mol). The reaction mixture was stirred at room temperature for 17 h then was checked by LCMS which showed the major isomer of C27 [LC/MS) m/z (M-tert-butyl group)⁺=321.9 at 0.87 min retention time] and the minor isomer of C28 [(LC/MS) m/z (M+H)⁺=378.2 at 0.94 min retention time]. The suspension was concentrated in vacuo then dissolved in DCM and purified by column chromatography (silica gel, 0-40% EtOAc:heptane) to provide the major isomer of C27 (eluted at 30-40% EtOAc:heptane) (0.344 g, 60.7% yield) as a yellow oil. (LC/MS) m/z (M-tert-butyl group)⁺=322.2. ¹H NMR (600 MHz, CDCl₃) δ 4.69 (q, 2H), 4.62 (br s, 2H), 4.41 (q, 2H), 3.77-3.70 (m, 2H), 2.74-2.70 (m, 2H), 1.56-1.43 (m, 9H), 1.40 (t, 3H)

### Step 2. Preparation of ethyl 1-(2,2,2-trifluoroethyl)-4,5,6,7-tetrahydro-1H-pyrazolo[4,3-c]pyridine-3-carboxylate hydrochloride (P17)

To a solution of C27 (0.344 g, 0.912 mmol) in DCM (5.0 mL) was added 4M HCl in dioxane (1.4 mL). The reaction solution was stirred at 40° C. for 24 h then concentrated in vacuo to provide P17 (0.328 g, crude) as a white solid. (LC/MS) m/z (M+H)⁺=278.2. ¹H NMR (600 MHz, (CD₃)₂SO) δ 9.20 (br s, 1H), 5.31 (q, 2H), 4.32-4.22 (m, 4H), 3.39 (t, 2H), 2.99 (t, 2H), 1.29-1.26 (m, 3H).

### Preparation 18

### rac-Ethyl (R)-1,6-dimethyl-4,5,6,7-tetrahydro-1H-pyrazolo[4,3-c]pyridine-3-carboxylate (P18)

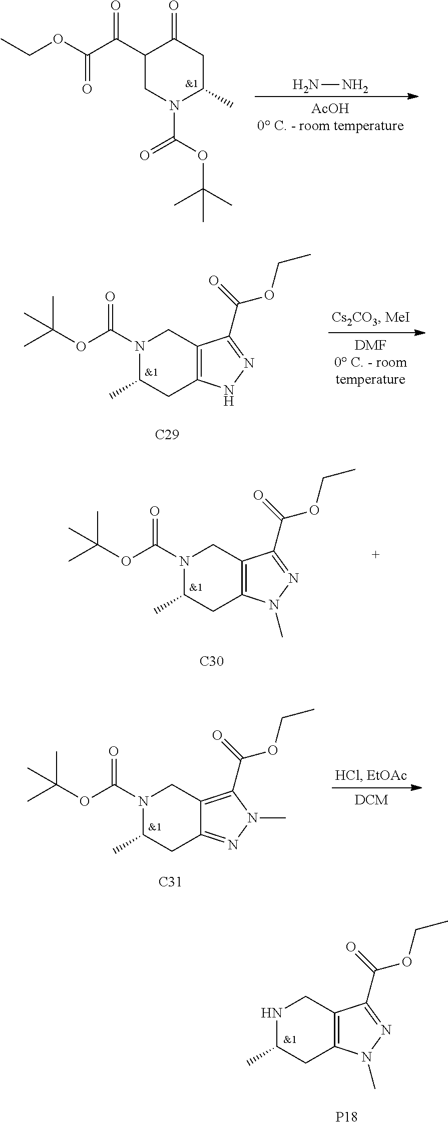

### Step 1. Preparation of rac-5-(tert-butyl) 3-ethyl (R)-6-methyl-1,4,6,7-tetrahydro-5H-pyrazolo[4,3-c]pyridine-3,5-dicarboxylate (C29)

At 0° C., to a suspension of rac-tert-butyl (2R)-5-(2-ethoxy-2-oxoacetyl)-2-methyl-4-oxopiperidine-1-carboxylate (CAS: 2386748-67-8; 24 g, 77 mmol) in AcOH (120 mL) was added hydrazine monohydrate (CAS: 7803-57-8; 14 g, 0.18 mol) dropwise via addition funnel. The reaction was stirred at room temperature for 1 h then concentrated in vacuo. The residue was diluted in EtOAc (200 mL) then washed with aqueous NaHCO₃. The organic layer was dried over Na₂SO₄, filtered, and concentrated in vacuo to provide C29 (23 g, crude) as an orange viscous liquid. The liquid was used directly in the next step without further purification. (LC/MS) m/z (M+H)⁺=310.2. ¹H NMR (400 MHz, CDCl₃) δ 10.21 (s, 1H), 5.06 (d, 1H), 4.85 (s, 1H), 4.44-4.28 (m, 2H), 4.15-4.10 (m, 1H), 2.96 (dd, 1H), 2.67-2.58 (m, 1H), 1.48-1.46 (m, 9H), 1.44-1.32 (m, 3H), 1.10 (d, 3H).

### Step 2. Preparation of rac-5-(tert-butyl) 3-ethyl (R)-1,6-dimethyl-1,4,6,7-tetrahydro-5H-pyrazolo[4,3-c]pyridine-3,5-dicarboxylate (C30) and rac-(R)-2-((3-(ethoxycarbonyl)-2,6-dimethyl-4,5,6,7-tetrahydro-2H-pyrazolo[4,3-c]pyridine-5-carbonyl)oxy)-2-methylpropan-1-ylium (C31)

At 0° C., to a solution C29 (23 g, 74 mmol) in DMF (200 mL) was added Cs₂CO₃ (27 g, 82 mmol) and Mel (14 mL) dropwise. The reaction mixture was stirred at room temperature for 20 h then concentrated in vacuo. The residue was dissolved in MTBE (200 mL) then wash with H₂O (200 mL). The organic layer was dried over Na₂SO₄, filtered, and concentrated in vacuo to form an orange oil. The residue was purified by column chromatography (silica gel, 0-100% isopropyl acetate:heptane) to provide C30 (3.0 g, 25% yield) and C31 (1.6 g, 14% yield). C30: (LC/MS) m/z (M+H)⁺=324.2. ¹H NMR (400 MHz, CDCl₃) δ 5.12-4.92 (m, 1H), 4.87-4.72 (m, 1H), 4.41-4.23 (m, 2H), 4.11 (s, 3H), 4.10-4.02 (m, 1H), 2.90 (dd, 1H), 2.59-2.48 (m, 1H), 1.48-1.44 (m, 9H), 1.39-1.34 (m, 3H), 1.09 (d, 3H).

C31: (LC/MS) m/z (M+H)⁺=324.2. ¹H NMR (400 MHz, CDCl₃) δ 5.08-4.85 (m, 2H), 4.44-4.30 (m, 2H), 4.08 (d, 1H), 3.80 (s, 3H), 2.94-2.84 (m, 1H), 2.44-2.38 (m, 1H), 1.46 (s, 9H), 1.37 (t, 3H), 1.20 (d, 3H).

### Step 3. Preparation of rac-ethyl (R)-1,6-dimethyl-4,5,6,7-tetrahydro-1H-pyrazolo[4,3-c]pyridine-3-carboxylate (P18)

To a solution of C30 (0.460 g, 1.42 mmol) in DCM (5 mL) was added 1M HCl in EtOAc (5 mL). The reaction solution was stirred at room temperature for 6 h then concentrated in vacuo to provide P18 (0.325 g, crude) as a white foam, which was used directly in the next step without further purification.

### Preparation 19

### Ethyl 3-(1,1-difluoroethyl)-5,6,7,8-tetrahydroimidazo[1,5-a]pyrazine-1-carboxylate hydrochloride (P19)

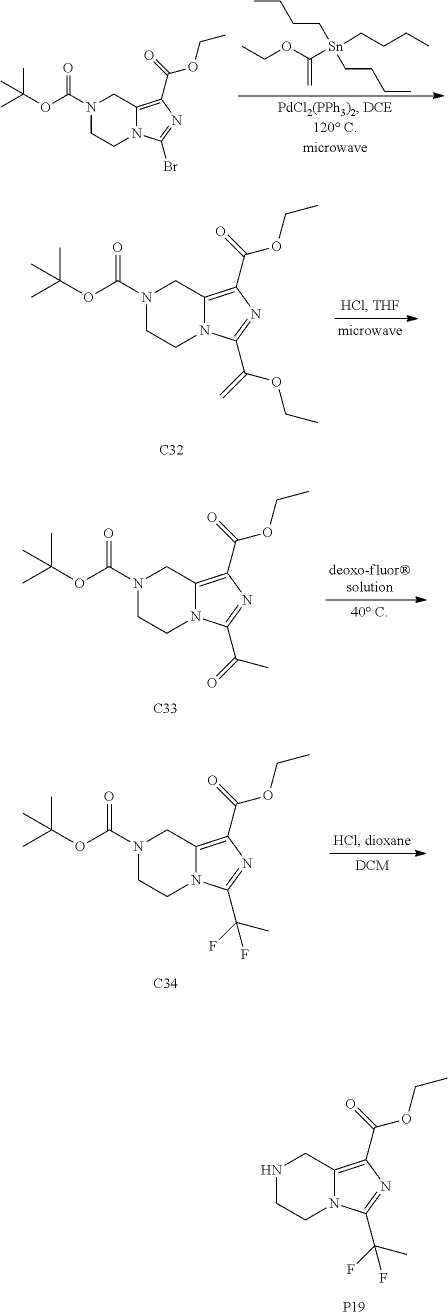

### Step 1. Preparation of 7-(tert-butyl) 1-ethyl 3-(1-ethoxyvinyl)-5,6-dihydroimidazo[1,5-a]pyrazine-1,7(8H)-dicarboxylate (C32)

To a solution of 7-(tert-butyl) 1-ethyl 3-bromo-5,6-dihydroimidazo[1,5-a]pyrazine-1,7(8H)-dicarboxylate (CAS: 2108354-93-2; 1.50 g, 4.01 mmol) in DCE (30 mL) was added tributyl(1-ethoxyvinyl)tin (CAS: 97674-02-7; 4.45 g, 12.3 mmol) and PdCl₂(PPh₃)₂ (0.422 g, 0.601 mmol). The reaction mixture was stirred in a microwave at 120° C. for 30 min then was concentrated in vacuo. The residue was purified by column chromatography (silica gel; 0-27% THF:PE) to provide C32 (0.960 g, 65.5% yield) as an oil. (LC/MS) m/z (M+H)⁺=366.1. ¹H NMR (400 MHz, CDCl₃) δ 5.06 (d, 1H), 4.90 (s, 2H), 4.42-4.33 (m, 2H), 4.18 (t, 2H), 3.90 (q, 2H), 3.79-3.71 (m, 3H), 1.50 (br s, 9H), 1.44-1.33 (m, 6H).

### Step 2. Preparation of 7-(tert-butyl) 1-ethyl 3-acetyl-5,6-dihydroimidazo[1,5-a]pyrazine-1,7(8H)-dicarboxylate (C33)

To a solution of C32 (0.660 g, 1.81 mmol) in THF (12 mL) was added 2M HCl (0.132 g, 3.61 mmol). The reaction mixture was stirred in the microwave for 16 h then the clear yellow solution was concentrated in vacuo. The residue was poured into saturated NaHCO₃ then extracted with EtOAc (3×20 mL). The combined organic layer was dried over MgSO₄, filtered, and concentrated in vacuo. The brown residue was purified by column chromatography (silica gel, 0-20% EtOAc:PE) to provide C33 (0.440 g, 81.1% yield) as a white solid. (LC/MS) m/z (M+H)⁺=338.3. ¹H NMR (400 MHz, CDCl₃) δ 4.95 (s, 2H), 4.47 (t, 2H), 4.41 (q, 2H), 3.79 (t, 2H), 2.70 (s, 3H), 1.50 (s, 9H), 1.41 (t, 3H).

### Step 3. Preparation of 7-(tert-butyl) 1-ethyl 3-(1,1-difluoroethyl)-5,6-dihydroimidazo[1,5-a]pyrazine-1,7(8H)-dicarboxylate (C34)

A solution of C33 (0.435 g, 1.29 mmol) in deoxo-Fluor™ solution (CAS: 202289-38-1; 10 mL) was stirred at 40° C. for 72 h. The reaction mixture was cooled to room temperature then extracted with DCM (2×20 mL). The combined organic layer was dried over MgSO₄, filtered, and concentrated in vacuo. The residue was purified by column chromatography (silica gel, 0-20% EtOAc:PE) to provide C34 (0.310 g, 66.9% yield) as a yellow gum. (LC/MS) m/z (M+H)⁺=360.1. ¹H NMR (400 MHz, CDCl₃) δ 4.92 (s, 2H), 4.38 (q, 2H), 4.25 (t, 2H), 3.83 (t, 2H), 2.19 (t, 3H), 1.50 (s, 9H), 1.39 (t, 3H).

### Step 4. Preparation of ethyl 3-(1,1-difluoroethyl)-5,6,7,8-tetrahydroimidazo[1,5-a]pyrazine-1-carboxylate hydrochloride (P19)

To a solution of C34 (0.310 g, 0.863 mmol) in DCM (3 mL) was added 2M HCl in dioxane (6 mL). The reaction mixture was stirred at room temperature for 16 h then concentrated in vacuo and dried further by high vacuo to provide P19 (0.255 g, crude) as a gum. The gum was used directly in the next step without further purification.

### Preparation 20

### rac-Ethyl (R)-7-ethyl-4,5,6,7-tetrahydro-1H-pyrazolo[4,3-c]pyridine-3-carboxylate hydrochloride (P20)

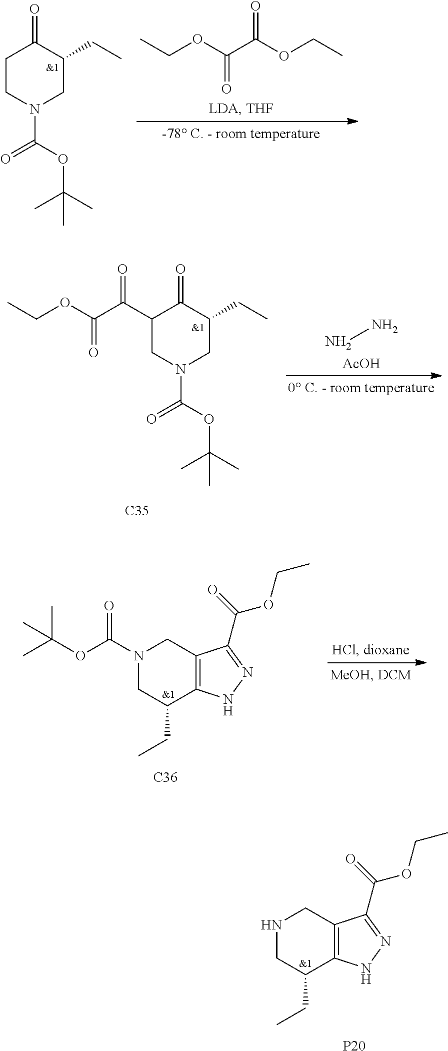

### Step 1. Preparation of rac-tert-butyl (5R)-3-(2-ethoxy-2-oxoacetyl)-5-ethyl-4-oxopiperidine-1-carboxylate (C35)

At −78° C. under nitrogen gas in a 250 mL three-neck round bottom flask, LDA (4.24 g, 39.6 mmol) was added to THF (25 mL). To the solution at −78° C., tert-butyl 3-ethyl-4-oxopiperidine-1-carboxylate (CAS: 117565-57-8; 9.00 g, 39.6 mmol) in THF (60 mL) was added dropwise then stirred at −78° C. for 1 h. After the stir, a solution of diethyl oxalate (5.79 g, 39.6 mmol) in THF (20 mL) was added dropwise at −78° C. then stirred at −78° C. for 1 h. The reaction mixture warmed to room temperature then stirred for 16 h. The suspension was cooled to 0° C. then quenched with 1M aqueous KHSO₄ (150 mL) and filtered. The filtrate was extracted with EtOAc (2×200 mL). The combined organic layer was dried over Na₂SO₄, filtered, and concentrated in vacuo to provide C35 (13.0 g, crude) as a yellow gum. The gum was used directly in the next step without further purification. (LC/MS) m/z (M-tert-butyl group)⁺=272.0. ¹H NMR (400 MHz, CDCl₃) δ 15.42 (br s, 1H), 4.53-4.16 (m, 4H), 3.68-3.32 (m, 2H), 2.44-2.32 (m, 1H), 1.84-1.67 (m, 1H), 1.53-1.42 (m, 2H), 1.41-1.39 (m, 9H), 1.31 (t, 3H), 0.97 (t, 3H).

### Step 2. Preparation of rac-5-(tert-butyl) 3-ethyl (R)-7-ethyl-1,4,6,7-tetrahydro-5H-pyrazolo[4,3-c]pyridine-3,5-dicarboxylate (C36)

The same procedure was followed from Preparation 18, step 1 with C35 (6.00 g, 18.3 mmol) to provide C36 (8.89 g, crude) as a yellow gum, which was used directly in the next step without further purification. (LC/MS) m/z (M+H)⁺=324.1. ¹H NMR (400 MHz, CDCl₃) δ 8.00 (br s, 1H), 4.93-4.44 (m, 2H), 4.37 (q, 2H), 3.83-3.68 (m, 1H), 3.57-3.41 (m, 1H), 2.88-2.71 (m, 1H), 1.84-1.70 (m, 1H), 1.62-1.51 (m, 2H), 1.50-1.48 (m, 9H), 1.38 (t, 3H), 1.05 (t, 3H).

### Step 3. Preparation of rac-ethyl (R)-7-ethyl-4,5,6,7-tetrahydro-1H-pyrazolo[4,3-c]pyridine-3-carboxylate hydrochloride (P20)

To a suspension of C36 (0.400 g, 1.24 mmol) in DCM (6 mL) and MeOH (2 mL) was added 2M HCl in dioxane (3 mL). The reaction mixture was stirred at room temperature for 2 h then an additional portion of 2M HCl in dioxane (3 mL) was added. The suspension was stirred for 16 h at room temperature then concentrated in vacuo to provide P20 (0.276 g, crude) as a yellow solid. The solid was used directly in the next step without further purification. ¹H NMR (400 MHz, (CD₃)₂SO) δ 9.68 (br s, 2H), 4.37-4.11 (m, 4H), 3.52-3.44 (m, 1H), 3.14-3.06 (m, 1H), 3.05-2.94 (m, 1H), 1.99-1.85 (m, 1H), 1.67-1.53 (m, 1H), 1.29 (t, 3H), 0.94 (t, 3H).

### Preparation 21

### rac-(R)-7-Ethyl-1-methyl-4,5,6,7-tetrahydro-1H-pyrazolo[4,3-c]pyridine-3-carboxylic acid hydrochloride (P21)

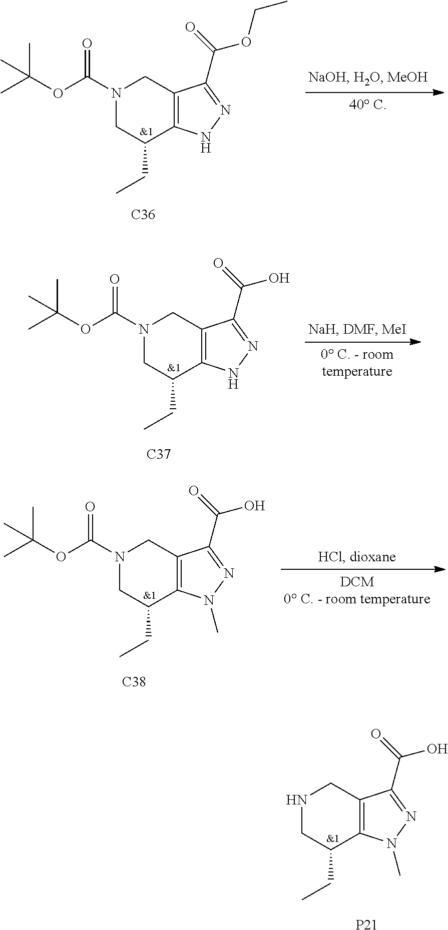

### Step 1. Preparation of rac-(R)-5-(tert-butoxycarbonyl)-7-ethyl-4,5,6,7-tetrahydro-1H-pyrazolo[4,3-c]pyridine-3-carboxylic acid (C37)

To a suspension of C36 (2.00 g, 6.18 mmol) in MeOH (6.6 mL) and H₂O (6.6 mL) was added NaOH (0.495 g, 12.4 mmol). The reaction mixture was stirred at 40° C. for 2 h then was diluted with H₂O (20 mL). The diluted reaction mixture was cooled to 0° C. then acidified with 1M aqueous HCl to pH˜3 and filtered. The filter cake was collected to provide C37 (1.60 g, crude) as a white solid. The solid was used directly in the next step without further purification. (LC/MS) m/z (M+H)⁺=296.1. ¹H NMR (400 MHz, (CD₃)₂SO) δ 13.15 (br s, 1H), 4.73-4.24 (m, 2H), 3.74-3.41 (m, 2H), 2.78-2.67 (m, 1H), 1.70 (br s, 1H), 1.49-1.40 (m, 10H), 1.02 (t, 3H).

### Step 2. Preparation of rac-(R)-5-(tert-butoxycarbonyl)-7-ethyl-1-methyl-4,5,6,7-tetrahydro-1H-pyrazolo[4,3-c]pyridine-3-carboxylic acid (C38)

The same procedure was followed from Preparation 3, step 2 with C37 (1.00 g, 3.39 mmol) and Mel (0.577 g, 4.06 mmol) to provide C38 (0.850 g, crude) as a white solid, which was used directly in the next step without further purification. (LC/MS) m/z (M+H)⁺=310.1. ¹H NMR (400 MHz, (CD₃)₂SO) δ 12.65 (br s, 1H), 5.01-4.75 (m, 1H), 4.34-3.88 (m, 2H), 3.79 (s, 3H), 3.07-2.67 (m, 2H), 1.63-1.50 (m, 1H), 1.43 (s, 9H), 1.36-1.23 (m, 1H), 1.08-0.95 (m, 3H).

### Step 3. Preparation of rac-(R)-7-ethyl-1-methyl-4,5,6,7-tetrahydro-1H-pyrazolo[4,3-c]pyridine-3-carboxylic acid hydrochloride (P21)

At 0° C., to a solution of C38 (0.300 g, 0.970 mmol) in DCM (1 mL) was added HCl in dioxane (2 mL). The reaction mixture was warmed to room temperature and stirred for 16 h then concentrated in vacuo to provide P21 (0.236 g, crude) as a yellow solid. ¹H NMR (400 MHz, (CD₃)₂SO) δ 12.97 (br s, 1H), 9.70 (br s, 1H), 9.13 (br s, 1H), 4.27-4.03 (m, 2H), 3.86 (s, 3H), 3.32-3.08 (m, 3H), 1.81-1.66 (m, 2H), 0.97 (t, 3H).

### Preparation 22

### 2-Chloro-5-(1-methyl-1H-imidazol-2-yl)pyridine (P22)

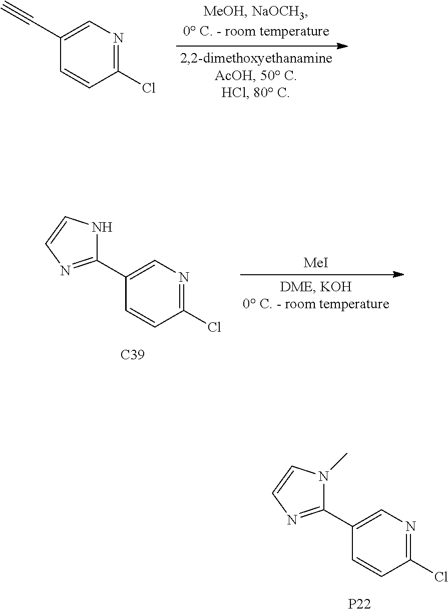

### Step 1. Preparation of 2-chloro-5-(1H-imidazol-2-yl)pyridine (C39)

At 0° C., to a solution of 2-chloro-5-ethynylpyridine (CAS: 263012-63-1, 94.0 g, 680. mmol) in MeOH (1.5 L) was added NaOCH₃ (5.50 g, 102 mmol). The reaction was stirred at room temperature for 1 h before 2,2-dimethoxyethanamine (71.3 g, 678 mmol) and AcOH (81.5 g, 1.36 mol) were added. The reaction was stirred at 50° C. for 1 h then cooled to room temperature before 6M HCl (74.2 g, 2.04 mol) was added. The reaction was stirred at 80° C. for 5 h then concentrated in vacuo. The residue was extracted with EtOAc (2×500 mL). The aqueous layer was adjusted to pH=10 with saturated aqueous Na₂CO₃ solution which caused white solid precipitate to form. The suspension was filtered then the filter cake was washed with H₂O (3×100 mL). The filter cake was collected then lyophilized to provide C39 (88.0 g, 72.2% yield) as a white solid. The solid was used directly in the next step without additional purification. (LC/MS) m/z (M+H)⁺=180.0. ¹H NMR (400 MHz, (CD₃)₂SO) δ 12.77 (br s, 1H), 8.94 (d, 1H), 8.31 (dd, 1H), 7.62 (d, 1H), 7.22 (s, 2H).

### Step 2. Preparation of 2-chloro-5-(1-methyl-1H-imidazol-2-yl)pyridine (P22)

To a solution of C39 (44.0 g, 204 mmol) in DME (440 mL) was added KOH (24.0 g, 428 mmol) at room temperature then stirred for 1 h and 40 min. The reaction mixture was cooled to 0° C. before Mel (57.8 g, 407 mmol) was added then stirred at 0° C. for 1 h. The white suspension was filtrated then concentrated in vacuo. The residue was washed with H₂O (300 mL) and stirred for 30 min. The reaction mixture was filtered, and the filter cake was concentrated in vacuo to provide P22 (34.8 g, 88.8% yield) as a white solid. The solid was used directly in the next step without additional purification. (LC/MS) m/z (M+H)⁺=194.0. ¹H NMR (400 MHz, (CD₃)₂SO) δ 8.79-8.76 (m, 1H), 8.20 (dd, 1H), 7.68-7.64 (m, 1H), 7.37 (d, 1H), 7.08 (d, 1H), 3.83 (s, 3H).

### Preparation 23

### rac-Ethyl (R)-1-ethyl-7-methyl-4,5,6,7-tetrahydro-1H-pyrazolo[4,3-c]pyridine-3-carboxylate trifluoroacetate (P23)

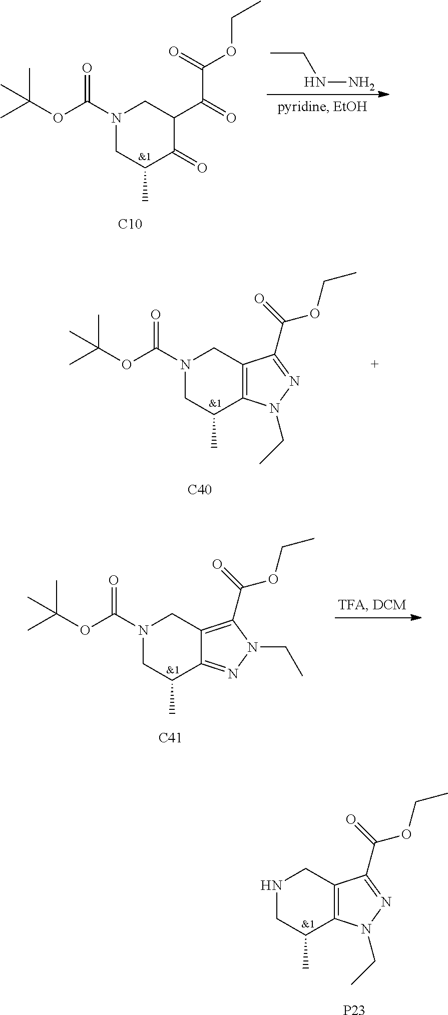

### Step 1. Preparation of rac-5-(tert-butyl) 3-ethyl (R)-1-ethyl-7-methyl-1,4,6,7-tetrahydro-5H-pyrazolo[4,3-c]pyridine-3,5-dicarboxylate (C40) and rac-5-(tert-butyl) 3-ethyl (R)-2-ethyl-7-methyl-2,4,6,7-tetrahydro-5H-pyrazolo[4,3-c]pyridine-3,5-dicarboxylate (C41)

A suspension of C10 (1.00 g, 3.19 mmol) and ethylhydrazine dihydrochloride (CAS: 49540-34-3; 0.425 g, 3.19 mmol) in EtOH (20 mL) and pyridine (1.11 g, 14.0 mmol) was stirred at room temperature for 16 h. The reaction mixture was concentrated in vacuo then purified by column chromatography (silica gel, 0-40% EtOAc:PE) to provide C41 (0.290 g, 26.9% yield) as a light-yellow solid and C40 (0.700 g, 65.0% yield) as a light-yellow gum.

C40: (LC/MS) m/z (M+H)⁺=338.2; ¹H NMR (400 MHz, CDCl₃) δ 5.20-4.88 (m, 1H), 4.44-4.32 (m, 2H), 4.29-3.97 (m, 4H), 3.16 (d, 1H), 3.00-2.88 (m, 1H), 1.52-1.44 (m, 12H), 1.38 (t, 3H), 1.25 (d, 3H).

C41: (LC/MS) m/z (M+H)⁺=338.2; ¹H NMR (400 MHz, CDCl₃) δ 4.75-4.46 (m, 4H), 4.33 (q, 2H), 3.96-3.64 (m, 1H), 3.36-3.08 (m, 1H), 3.00-2.91 (m, 1H), 1.50-1.44 (m, 9H), 1.39 (q, 6H), 1.26 (d, 3H).

### Step 2. Preparation of rac-ethyl (R)-1-ethyl-7-methyl-4,5,6,7-tetrahydro-1H-pyrazolo[4,3-c]pyridine-3-carboxylate trifluoroacetate (P23)

To a solution of C40 (0.250 g, 0.741 mmol) in DCM (4 mL) was added TFA (2 mL). The reaction mixture was stirred at room temperature for 2 h then concentrated in vacuo to provide P23 (0.260 g, crude) as a colorless gum, which was used directly in the next step without further purification.

### Preparation 24

### rac-Ethyl (4R,8S)-1-methyl-4,5,6,7,8,9-hexahydro-1H-4,8-epiminocycloocta[c]pyrazole-3-carboxylate (P24)

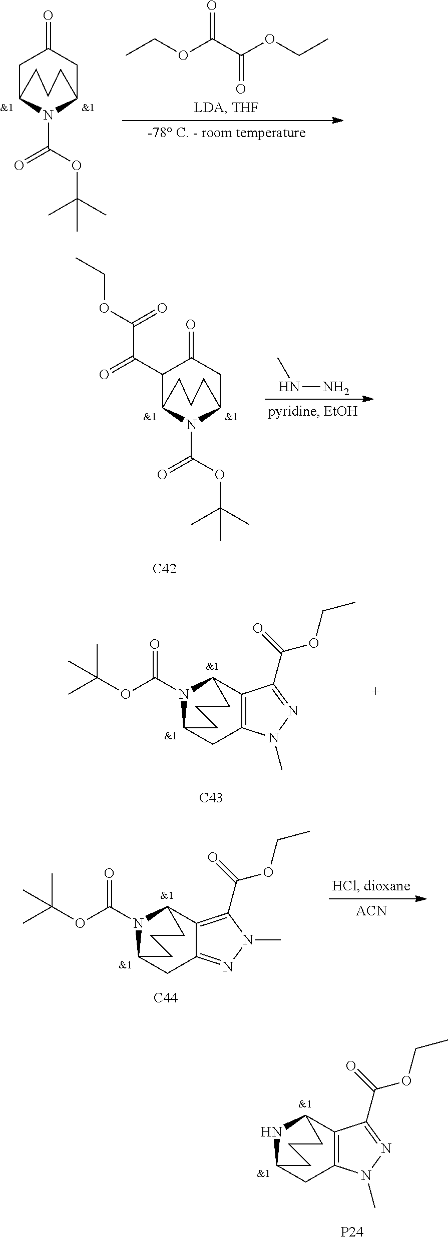

### Step 1. Preparation of rac-tert-butyl (1R,5S)-2-(2-ethoxy-2-oxoacetyl)-3-oxo-9-azabicyclo[3.3.1]nonane-9-carboxylate (C42)

At −78° C., LDA (1.07 g, 10.0 mmol) was added to THF (50 mL). To the solution at −78° C., tert-butyl (1R,5S)-3-oxo-9-azabicyclo[3.3.1]nonane-9-carboxylate (CAS: 512822-27-4; 2.00 g, 8.36 mmol) in THF (50 mL) was added dropwise and then stirred at −78° C. for 1 h. After the stir, a solution of diethyl oxalate (1.28 g, 8.78 mmol) in THF (20 mL) was added dropwise at −78° C. and then stirred at −78° C. for 30 min. The reaction mixture was warmed to room temperature, stirred for 1 h, and then concentrated in vacuo. The residue was purified by column chromatography (silica gel, 2-16% EtOAc:PE) to provide C42 (0.750 g, 26.4% yield) as a light yellow oil. (LC/MS) m/z (M+H)⁺=340.1 ¹H NMR (400 MHz, CDCl₃) δ 5.49 (d, 1H), 4.77-4.48 (m, 2H), 4.36 (q, 2H), 2.98-2.81 (m, 1H), 2.44-2.31 (m, 1H), 1.86-1.72 (m, 3H), 1.69-1.58 (m, 3H), 1.50-1.42 (m, 9H), 1.39 (t, 3H).

### Step 2. Preparation of rac-10-(tert-butyl) 3-ethyl (4R,8S)-1-methyl-4,5,6,7,8,9-hexahydro-1H-4,8-epiminocycloocta[c]pyrazole-3,10-dicarboxylate (C43) and rac-10-(tert-butyl) 3-ethyl (4R,8S)-2-methyl-4,5,6,7,8,9-hexahydro-2H-4,8-epiminocycloocta[c]pyrazole-3,10-dicarboxylate (C44)

To a solution of C42 (0.650 g, 2.17 mmol) in EtOH (20 mL) was added methylhydrazine dihydrochloride (CAS: 55330-60-4; 0.179 g, 2.17 mmol) and pyridine (0.859 g, 10.9 mmol). The reaction mixture was stirred at room temperature for 16 h and then concentrated in vacuo. The residue was purified by column chromatography (silica gel, 12-100% EtOAc:PE) to provide C43 (0.390 g, 51.4% yield) as an oil and C44 (0.150 g, 19.8% yield).

C43: (LC/MS) m/z (M+H)⁺=350.1. ¹H NMR (400 MHz, CDCl₃) δ 5.68-5.53 (m, 1H), 4.79-4.57 (m, 1H), 4.46-4.28 (m, 2H), 3.80 (s, 3H), 3.11-2.95 (m, 1H), 2.43 (d, 1H), 1.89-1.69 (m, 4H), 1.63-1.55 (m, 1H), 1.50-1.41 (m, 10H), 1.40-1.34 (m, 3H).

C44: (LC/MS) m/z (M+H)⁺=350.2. ¹H NMR (400 MHz, CDCl₃) δ 5.68-5.48 (m, 1H), 4.78-4.53 (m, 1H), 4.45-4.19 (m, 2H), 4.15-4.07 (m, 3H), 3.19-3.00 (m, 1H), 2.63-2.52 (m, 1H), 1.90-1.58 (m, 5H), 1.44 (s, 10H), 1.38 (t, 3H).

### Step 3. Preparation of rac-ethyl (4R,8S)-1-methyl-4,5,6,7,8,9-hexahydro-1H-4,8-epiminocycloocta[c]pyrazole-3-carboxylate (P24)

To a solution of C43 (0.222 g, 0.636 mmol) in ACN (6 mL) was added HCl in dioxane (0.116 g, 3.18 mmol). The reaction mixture was stirred at room temperature for 16 h then concentrated in vacuo to provide P24 (0.150 g, crude) as a white solid. The solid was used directly in the next step without further purification.

### Preparation P25

### Ethyl 7,7-difluoro-1-methyl-4,5,6,7-tetrahydro-1H-pyrazolo[4,3-c]pyridine-3-carboxylate hydrochloride (P25)

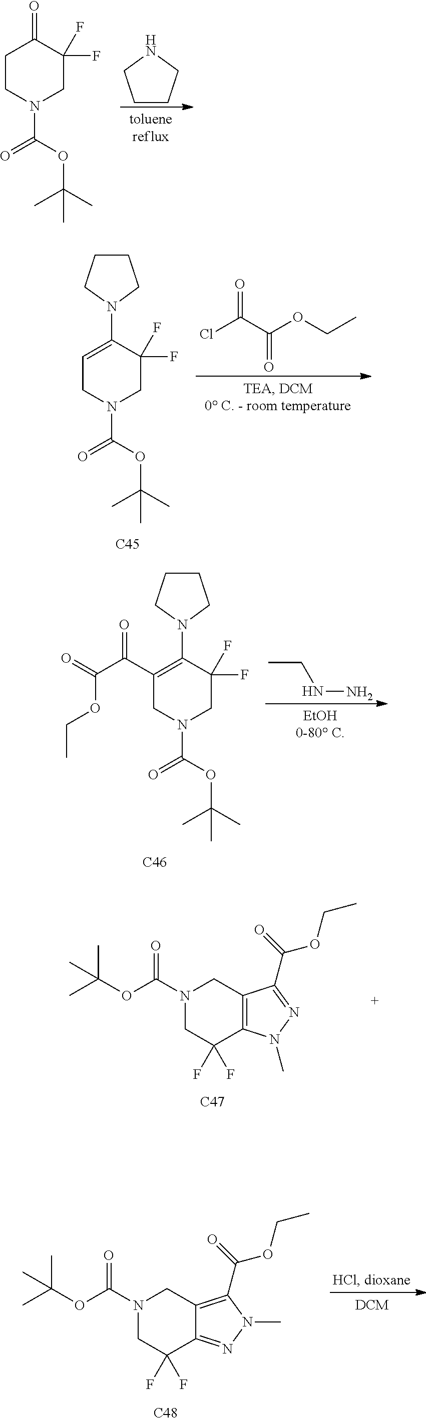

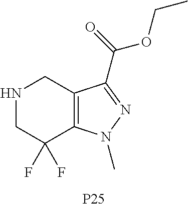

### Step 1. Preparation of tert-butyl 3,3-difluoro-4-(pyrrolidin-1-yl)-3,6-dihydropyridine-1(2H)-carboxylate (C45)

To a solution of tert-butyl 3,3-difluoro-4-oxopiperidine-1-carboxylate (CAS: 1215071-17-2; 3.00 g, 11.8 mmol) in toluene (40 mL) was added pyrrolidine (0.927 g, 13.0 mmol). The reaction mixture was heated for 20 h using a Deans and Stark trap to remove the H₂O liberated during the reaction, then concentrated in vacuo to provide C45 (3.42 g, crude) as a brown solid. The solid was used directly in the next step without further purification. ¹H NMR (400 MHz, CDCl₃) δ 4.66-4.46 (m, 1H), 4.14-4.05 (m, 2H), 3.90 (t, 2H), 3.20-3.13 (m, 4H), 1.97-1.88 (m, 4H), 1.53 (s, 9H).

### Step 2. Preparation of tert-butyl 5-(2-ethoxy-2-oxoacetyl)-3,3-difluoro-4-(pyrrolidin-1-yl)-3,6-dihydropyridine-1(2H)-carboxylate (C46)

A solution of C45 (3.42 g, 11.9 mmol) in DCM (50 mL) was degassed with nitrogen gas for 10 min. The reaction mixture was cooled to 0° C. before ethyl oxalyl chloride (1.94 g, 14.2 mmol) was added dropwise over 15 min. The temperature was maintained between (0-5) ° C. throughout the addition. Following the addition, TEA (1.80 g, 17.8 mmol) was added dropwise over 5 min. The reaction mixture was warmed to room temperature and stirred for 5 h, then diluted with H₂O (300 mL). The diluted reaction mixture was extracted with DCM (3×100 mL). The combined organic layer was dried with Na₂SO₄, filtered, and concentrated in vacuo. The residue was purified by column chromatography (silica gel, 0-25% EtOAc:PE) to provide C46 (2.98 g, 64.7% yield) as a yellow solid. (LC/MS) m/z (M+H)⁺=389.2. ¹H NMR (400 MHz, CDCl₃) δ 4.41 (s, 2H), 4.30 (q, 2H), 3.89 (t, 2H), 3.49-3.41 (m, 4H), 2.00-1.94 (m, 4H), 1.48 (s, 9H), 1.35 (t, 3H).

### Step 3. Preparation of 5-(tert-butyl) 3-ethyl 7,7-difluoro-1-methyl-1,4,6,7-tetrahydro-5H-pyrazolo[4,3-c]pyridine-3,5-dicarboxylate (C47) and 2-((3-(ethoxycarbonyl)-7,7-difluoro-2-methyl-4,5,6,7-tetrahydro-2H-pyrazolo[4,3-c]pyridine-5-carbonyl)oxy)-2-methylpropan-1-ylium (C48)

The reaction was conducted in three batches and then combined for further purification. At 0° C., to a solution of C46 (1.09 g, 2.81 mmol) in EtOH (10 mL) was added methylhydrazine dihydrochloride (CAS: 55330-60-4; 0.402 g, 3.38 mmol) dropwise over 5 min then the reaction was sealed and stirred at 80° C. for 2 h to form the first batch.

The second batch of the same reaction was conducted with C46 (0.500 g, 1.29 mmol), and the third batch was conducted with C46 (0.100 g, 0.257 mmol). The batches were combined and then diluted with H₂O (60 mL). The diluted reaction mixture was extracted with EtOAc (3×50 mL). The combined organic layer was dried over Na₂SO₄, filtered, and concentrated in vacuo. The residue was purified by column chromatography (silica gel, 0-15% EtOAc:PE) to provide C48 (0.756 g, 50.4% yield) as brown oil and an impure C47 as a residue. The residue, C47, was purified by reverse phase HPLC (C₁₈ 150 mm×40 mm×5 μm column, Mobile Phase A: H₂O (0.05% NH₄OH—NH₄HCO₃)/Mobile Phase B: ACN, 32 to 72% of Mobile Phase B over 9 min, 100% of Mobile Phase B hold 2 min, flow rate=60 mL/min) and lyophilized to provide C47 (0.416 g, 27.7% yield) as a colorless oil.

C47: (LC/MS) m/z (M-tert-butyl group)⁺=290.2; ¹H NMR (400 MHz, CDCl₃) δ 4.69 (br s, 2H), 4.41 (q, 2H), 4.09-3.96 (m, 5H), 1.49 (s, 9H), 1.40 (t, 3H).

C48: (LC/MS) m/z (M-tert-butyl group)⁺=290.2; ¹H NMR (400 MHz, CDCl₃) δ 4.73-4.63 (m, 2H), 4.37 (q, 2H), 4.23 (s, 3H), 4.06-3.95 (m, 2H), 1.49 (s, 9H), 1.40 (t, 3H).

### Step 4. Preparation of ethyl 7,7-difluoro-1-methyl-4,5,6,7-tetrahydro-1H-pyrazolo[4,3-c]pyridine-3-carboxylate hydrochloride (P25)

To a reaction mixture of C47 (0.416 g, 1.20 mmol) in DCM (5 mL) was added HCl in dioxane (1.76 g, 48.2 mmol) and then stirred at room temperature for 2.5 h. The reaction mixture was concentrated in vacuo to provide P25 (295 mg, crude) as a light-yellow solid. The solid was used directly in the next step without further purification. (LC/MS) m/z (M+H)⁺=246.1. ¹H NMR (400 MHz, CD₃OD) δ 4.55 (t, 2H), 4.40 (q, 2H), 4.15-4.02 (m, 5H), 1.39 (t, 3H).

### Preparation 26

### rac-Ethyl (4R,8S)-1-methyl-1,4,5,7,8,9-hexahydro-4,8-epiminooxocino[5,4-c]pyrazole-3-carboxylate hydrochloride (P26)

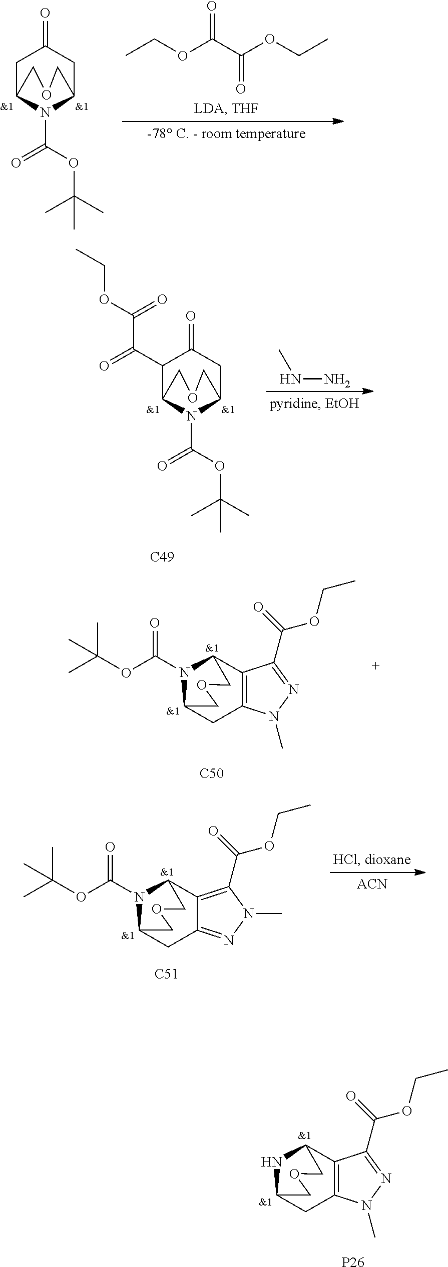

### Step 1. Preparation of rac-tert-butyl (1R,5S)-6-(2-ethoxy-2-oxoacetyl)-7-oxo-3-oxa-9-azabicyclo[3.3.1]nonane-9-carboxylate (C49)

The same procedure was followed from Preparation 20, step 1 with tert-butyl 7-oxo-3-oxa-9-azabicyclo[3.3.1]nonane-9-carboxylate (CAS: 280761-97-9; 1.80 g, 7.46 mmol) to provide C49 (2.50 g, crude) as an orange oil, which was used directly in the next step without further purification.

### Step 2. Preparation of rac-10-(tert-butyl) 3-ethyl (4R,8S)-1-methyl-1,4,5,7,8,9-hexahydro-4,8-epiminooxocino[5,4-c]pyrazole-3,10-dicarboxylate (C50) and rac-10-(tert-butyl) 3-ethyl (4R,8S)-2-methyl-2,4,5,7,8,9-hexahydro-4,8-epiminooxocino[5,4-c]pyrazole-3,10-dicarboxylate (C51)

The reaction was conducted in two batches and then combined. A suspension of C49 (2.50 g, 7.32 mmol) and methylhydrazine dihydrochloride (CAS: 55330-60-4; 0.871 g, 7.32 mmol) in EtOH (40 mL) and pyridine (2.55 g, 32.2 mmol) was stirred at room temperature for 3 h to form the first batch. The reaction mixture of the first batch was checked by LCMS, which showed the major isomer of C50 [LC/MS) m/z (M+H)⁺=352.3 at 0.827 min retention time (analytical LCMS conditions: Chromolith Flash Reverse Phase-18e 25-3 mm column; Mobile Phase A: 1.5 mL/4 L TFA in H₂O/Mobile Phase B: 0.75 mL/4 L TFA in ACN, gradient 5-95% of Mobile Phase B over 0.7 min then held at 95% of Mobile Phase B for 0.4 min; flow rate: 1.5 mL/min column temperature: 50° C.)] and the minor isomer of C51 [LC/MS) m/z (M+H)⁺=352.3 at 0.880 min retention time (analytical LCMS conditions: Chromolith Flash Reverse Phase-18e 25-3 mm column; Mobile Phase A: 1.5 mL/4 L TFA in H₂O/Mobile Phase B: 0.75 mL/4 L TFA in ACN, gradient 5-95% of Mobile Phase B over 0.7 min then held at 95% of Mobile Phase B for 0.4 min; flow rate: 1.5 mL/min column temperature: 50° C.)].

The second batch of the same reaction was conducted with C49 (0.200 g, 0.586 mmol). The two batches were combined, concentrated in vacuo, and purified by column chromatography (silica gel, 0-50% EtOAc:PE) to provide C50 (0.750 g, 27.0% yield) as a yellow solid. (LC/MS) m/z (M+H)⁺=352.3. ¹H NMR (400 MHz, CDCl₃) δ 5.55-5.30 (m, 1H), 4.53-4.27 (m, 3H), 3.91-3.64 (m, 7H), 3.21-2.92 (m, 1H), 2.71-2.59 (m, 1H), 1.51-1.30 (m, 12H).

### Step 3. Preparation of rac-ethyl (4R,8S)-1-methyl-1,4,5,7,8,9-hexahydro-4,8-epiminooxocino[5,4-c]pyrazole-3-carboxylate hydrochloride (P26)

To a solution of C50 (0.750 g, 2.14 mmol) in DCM (5 mL) was added 2M HCl in dioxane (0.390 g, 10.7 mmol). The reaction mixture was stirred at room temperature for 2 h and then concentrated in vacuo. The residue was dissolved in DCM (5 mL) and then concentrated in vacuo (2×) to provide P26 (0.675 g, crude) as a yellow solid. The solid was used directly in the next step without further purification. ¹H NMR (400 MHz, CDCl₃) δ 10.83 (s, 1H), 9.85 (s, 1H), 5.07 (s, 1H), 4.45-4.20 (m, 4H), 4.01-3.84 (m, 5H), 3.82-3.73 (m, 1H), 3.66-3.55 (m, 1H), 2.98-2.86 (m, 1H), 1.38 (t, 3H).

### Preparation 27

### Ethyl 1-propyl-4,5,6,7-tetrahydro-1H-pyrazolo[4,3-c]pyridine-3-carboxylate (P27)

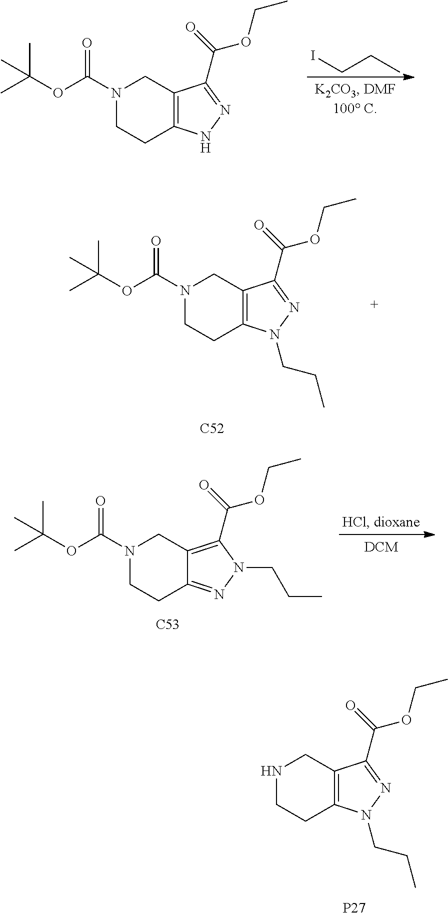

### Step 1. Preparation of 5-(tert-butyl) 3-ethyl 1-propyl-1,4,6,7-tetrahydro-5H-pyrazolo[4,3-c]pyridine-3,5-dicarboxylate (C52) and 2-((3-(ethoxycarbonyl)-2-propyl-4,5,6,7-tetrahydro-2H-pyrazolo[4,3-c]pyridine-5-carbonyl)oxy)-2-methylpropan-1-ylium (C53)

To a solution of 5-(tert-butyl) 3-ethyl 1,4,6,7-tetrahydro-5H-pyrazolo[4,3-c]pyridine-3,5-dicarboxylate (CAS: 518990-23-3; 0.81 g, 4.7 mmol) and 1-iodopropane (1.0 g, 3.4 mmol) in DMF (13 mL) was added K₂CO₃ (0.94 g, 6.7 mmol). The reaction mixture was stirred at 100° C. for 4 h, cooled to room temperature, and diluted with (1:1) EtOAc:H₂O (50 mL). The aqueous layer was extracted with EtOAc (3×25 mL). The combined organic layer was dried with Na₂SO₄, filtered, and concentrated in vacuo. The residue was purified by column chromatography (silica gel, 0-39% EtOAc:PE) to provide C53 (0.48 g, 42% yield) as a white solid and C52 (0.38 g, 33% yield) as a white solid.

C52: (LC/MS) m/z (M+H)⁺=338.3; ¹H NMR (400 MHz, CDCl₃) δ 4.61 (s, 2H), 4.39 (q, 2H), 4.03 (t, 2H), 3.76-3.67 (m, 2H), 2.73-2.63 (m, 2H), 1.92-1.80 (m, 2H), 1.48 (s, 9H), 1.39 (t, 3H), 0.91 (t, 3H).

C53: (LC/MS) m/z (M+H)⁺=338.3; ¹H NMR (400 MHz, CDCl₃) δ 4.65-4.56 (m, 2H), 4.49-4.43 (m, 2H), 4.33 (q, 2H), 3.68 (s, 2H), 2.74 (s, 2H), 1.90-1.78 (m, 2H), 1.49 (s, 9H), 1.39 (t, 3H), 0.92 (t, 3H).

### Step 2. Preparation of ethyl 1-propyl-4,5,6,7-tetrahydro-1H-pyrazolo[4,3-c]pyridine-3-carboxylate (P27)

To a reaction mixture of C52 (0.370 g, 1.10 mmol) in DCM (3 mL) was added 2M HCl in dioxane (0.400 g, 11.0 mmol) and then stirred at room temperature for 1 h and 40 min. The reaction mixture was concentrated in vacuo to provide P27 (0.300 g, crude) as a white solid. The solid was used directly in the next step without further purification. ¹H NMR (400 MHz, (CD₃)₂SO) δ 9.54 (s, 2H), 4.25 (q, 2H), 4.19 (s, 2H), 4.06 (t, 2H), 3.40-3.35 (m, 2H), 2.97 (t, 2H), 1.80-1.68 (m, 2H), 1.27 (t, 3H), 0.82 (t, 3H).

### Preparation 28

### Ethyl 1-(tetrahydro-2H-pyran-4-yl)-4,5,6,7-tetrahydro-1H-pyrazolo[4,3-c]pyridine-3-carboxylate hydrochloride (P28)

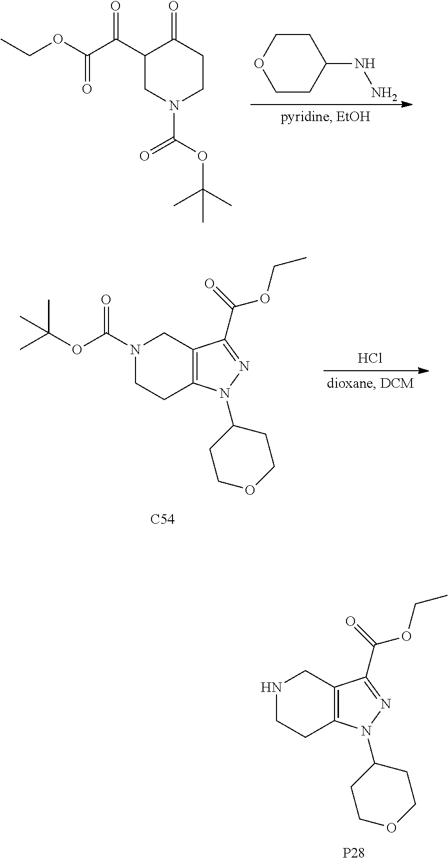

### Step 1. Preparation of 5-(tert-butyl) 3-ethyl 1-(tetrahydro-2H-pyran-4-yl)-1,4,6,7-tetrahydro-5H-pyrazolo[4,3-c]pyridine-3,5-dicarboxylate (C54)

The same procedure was followed from Preparation 2, step 1 with tert-butyl 3-(2-ethoxy-2-oxoacetyl)-4-oxopiperidine-1-carboxylate (CAS: 518990-24-4; 0.500 g, 1.67 mmol) and (tetrahydro-2H-pyran-4-yl)hydrazine hydrochloride (0.255 g, 1.67 mmol). The residue was purified by column chromatography (silica gel, 0-40% THF:PE) to provide C54 (0.492 g, 77.6% yield) as a yellow gum. (LC/MS) m/z (M+H)⁺=380.3. ¹H NMR (400 MHz, CDCl₃) δ 4.60 (s, 2H), 4.38 (q, 2H), 4.31-4.21 (m, 1H), 4.15-4.07 (m, 2H), 3.77-3.68 (m, 2H), 3.54-3.44 (m, 2H), 2.73 (t, 2H), 2.41-2.25 (m, 2H), 1.89-1.80 (m, 2H), 1.51-1.40 (m, 9H), 1.38 (t, 3H).

### Step 2. Preparation of ethyl 1-(tetrahydro-2H-pyran-4-yl)-4,5,6,7-tetrahydro-1H-pyrazolo[4,3-c]pyridine-3-carboxylate hydrochloride (P28)

To a solution of C54 (0.470 g, 1.24 mmol) in DCM (10 mL) was added 2M HCl in dioxane (0.973 g, 26.7 mmol). The reaction mixture was stirred at room temperature for 4 h then concentrated in vacuo to provide P28 (0.370 g, crude) as a yellow solid. The solid was used directly in the next step without further purification. (LC/MS) m/z (M+H)⁺=280.1. ¹H NMR (400 MHz, (CD₃)₂SO) δ 9.37 (s, 1H), 4.56-4.45 (m, 1H), 4.33-4.19 (m, 4H), 4.03-3.93 (m, 2H), 3.52-3.41 (m, 4H), 3.04 (t, 2H), 2.07-1.92 (m, 2H), 1.86-1.77 (m, 2H), 1.29 (t, 3H).

### Preparation 29

### rac-Ethyl (R)-1-(sec-butyl)-4,5,6,7-tetrahydro-1H-pyrazolo[4,3-c]pyridine-3-carboxylate hydrochloride (P29)

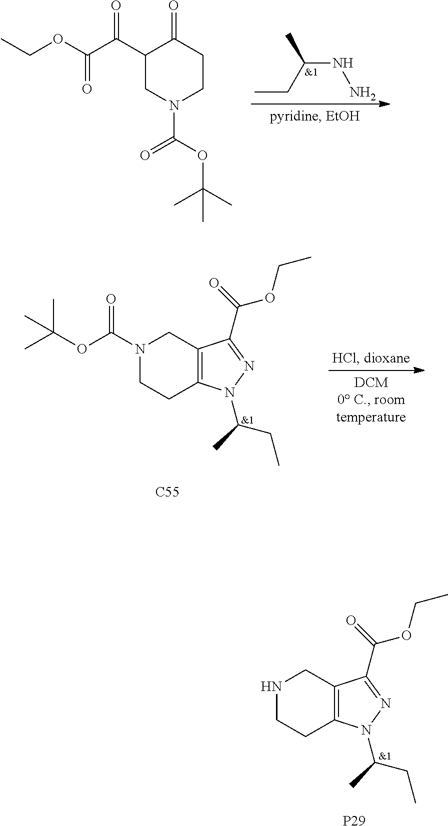

### Step 1. Preparation of rac-5-(tert-butyl) 3-ethyl (R)-1-(sec-butyl)-1,4,6,7-tetrahydro-5H-pyrazolo[4,3-c]pyridine-3,5-dicarboxylate (C55)

The same procedure was followed from Preparation 2, step 1 with tert-butyl 3-(2-ethoxy-2-oxoacetyl)-4-oxopiperidine-1-carboxylate (CAS: 518990-24-4; 0.400 g, 1.34 mmol) and sec-butylhydrazine dihydrochloride (CAS: 1177361-36-2, 0.215 g, 1.34 mmol). The residue was purified by column chromatography (silica gel, 50% EtOAc:PE) to provide C55 (0.382 g, 81.3% yield) as a gum. (LC/MS) m/z (M+H)⁺=352.1. ¹H NMR (400 MHz, (CD₃)₂SO) δ 4.53-4.40 (m, 2H), 4.30-4.17 (m, 3H), 3.68-3.54 (m, 2H), 2.71 (q, 2H), 1.87-1.66 (m, 2H), 1.41 (s, 9H), 1.37 (d, 3H), 1.29 (t, 3H), 0.69 (t, 3H).

### Step 2. Preparation of rac-ethyl (R)-1-(sec-butyl)-4,5,6,7-tetrahydro-1H-pyrazolo[4,3-c]pyridine-3-carboxylate hydrochloride (P29)

At 0° C., to a solution of C55 (0.382 g, 1.09 mmol) in DCM (2 mL) was added HCl in dioxane (4 mL). The reaction mixture was stirred at room temperature for 16 h then concentrated in vacuo to provide P29 (0.245 g, crude) as a solid. The solid was used directly in the next step without further purification.

### Preparation 30

### rac-Ethyl (4R,7S)-1-methyl-1,4,5,6,7,8-hexahydro-4,7-epiminocyclohepta[c]pyrazole-3-carboxylate (P30)

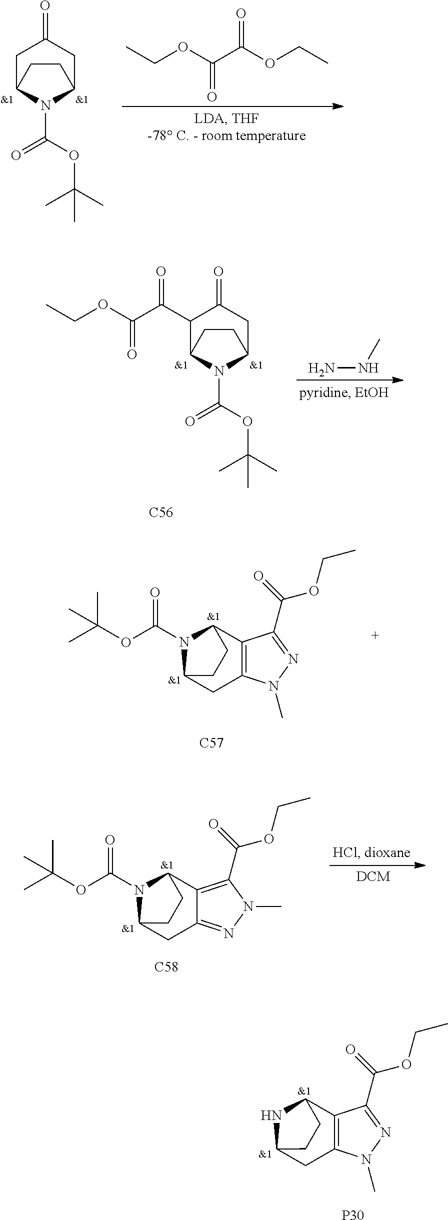

### Step 1. Preparation of rac-tert-butyl (1R,5S)-2-(2-ethoxy-2-oxoacetyl)-3-oxo-8-azabicyclo[3.2.1]octane-8-carboxylate (C56)

The same procedure was followed from Preparation 20, step 1, with tert-butyl (1R,5S)-8-oxo-3-azabicyclo[3.2.1]octane-3-carboxylate (CAS: 637301-19-0; 5.10 g, 22.6 mmol), with an additional purification added. The residue was purified by column chromatography (silica gel, 0-10% EtOAc:PE) to provide C56 (4.69 g, 63.7% yield) as a yellow oil.

### Step 2. Preparation of rac-9-(tert-butyl) 3-ethyl (4R,7S)-1-methyl-1,4,5,6,7,8-hexahydro-4,7-epiminocyclohepta[c]pyrazole-3,9-dicarboxylate (C57) and rac-9-(tert-butyl) 3-ethyl (4R,7S)-2-methyl-2,4,5,6,7,8-hexahydro-4,7-epiminocyclohepta[c]pyrazole-3,9-dicarboxylate (C58)

The same procedure was followed from Preparation 2, step 1 with C56 (4.69 g, 14.4 mmol) and methylhydrazine sulfate (CAS: 302-15-8, 2.08 g, 14.4 mmol). The reaction mixture was checked by LCMS, which showed the major isomer of C57 [LC/MS) m/z (M+H)⁺=336.2 at 0.846 min retention time (analytical LCMS conditions: Chromolith Flash Reverse Phase-18e 25-3 mm column; Mobile Phase A: 1.5 mL/4 L TFA in H₂O/Mobile Phase B: 0.75 mL/4 L TFA in ACN, gradient 5-95% of Mobile Phase B over 0.7 min then held at 95% of Mobile Phase B for 0.4 min; flow rate: 1.5 mL/min; column temperature: 50° C.)] and the minor isomer of C58 [LC/MS) m/z (M+H)⁺=336.2 at 0.904 min retention time (analytical LCMS conditions: Chromolith Flash Reverse Phase-18e 25-3 mm column; Mobile Phase A: 1.5 mL/4 L TFA in H₂O/Mobile Phase B: 0.75 mL/4 L TFA in ACN, gradient 5-95% of Mobile Phase B over 0.7 min then held at 95% of Mobile Phase B for 0.4 min; flow rate: 1.5 mL/min; column temperature: 50° C.)]. The purification was altered. The residue was purified by column chromatography (silica gel, 0-50% THF:PE) to provide C57 (1.40 g, 29.0% yield) as a light-yellow oil. ¹H NMR (400 MHz, CDCl₃) δ 5.42-5.33 (m, 1H), 4.64-4.35 (m, 3H), 3.77 (s, 3H), 3.29-3.08 (m, 1H), 2.41-2.23 (m, 2H), 2.21-2.10 (m, 1H), 1.93-1.85 (m, 1H), 1.55-1.48 (m, 1H), 1.44-1.35 (m, 12H).

### Step 3. Preparation of ethyl 1-methyl-1,4,5,6,7,8-hexahydro-4,7-epiminocyclohepta[c]pyrazole-3-carboxylate (P30)

To a solution of C57 (0.240 g, 0.716 mmol) in DCM (1 mL), 2M HCl in dioxane (3 mL) was added. The reaction mixture was stirred at room temperature for 2.5 h and then concentrated in vacuo to provide P30 (0.168 g, crude) as a grey solid. The solid was used directly in the next step without further purification.

### Preparation 31

### Ethyl 1-(cyclopropylmethyl)-4,5,6,7-tetrahydro-1H-pyrazolo[4,3-c]pyridine-3-carboxylate (P31)

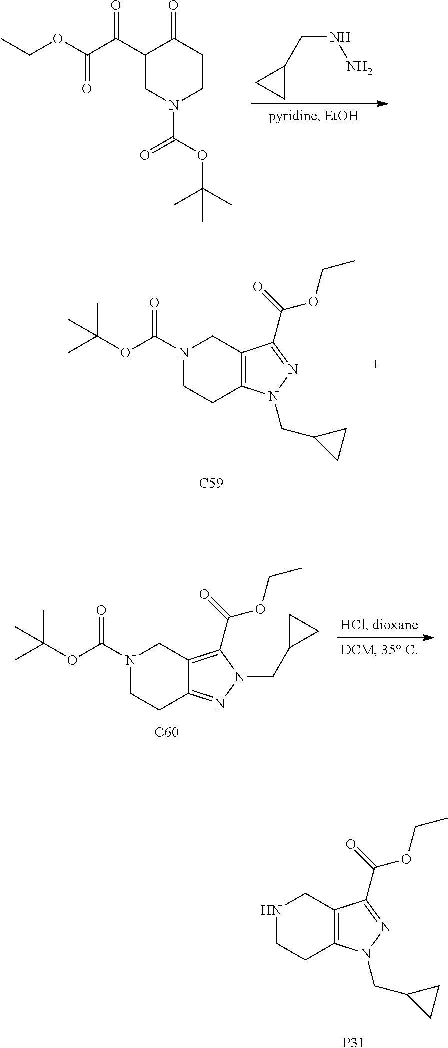

### Step 1. Preparation of 5-(tert-butyl) 3-ethyl 1-(cyclopropylmethyl)-1,4,6,7-tetrahydro-5H-pyrazolo[4,3-c]pyridine-3,5-dicarboxylate (C59) and 2-((2-(cyclopropylmethyl)-3-(ethoxycarbonyl)-4,5,6,7-tetrahydro-2H-pyrazolo[4,3-c]pyridine-5-carbonyl)oxy)-2-methylpropan-1-ylium (C60)

The same procedure was followed from Preparation 2, step 1 with tert-butyl 3-(2-ethoxy-2-oxoacetyl)-4-oxopiperidine-1-carboxylate (CAS: 518990-24-4; 2.00 g, 6.68 mmol) and (cyclopropylmethyl)hydrazine hydrochloride (CAS: 1181457-83-9, 0.655 g, 5.35 mmol). The residue was purified by column chromatography (silica gel, 0-60% EtOAc:PE) to provide an impure C60 (0.800 g) as a yellow oil and C59 (0.940 g, 40.3% yield) as a light-brown solid. C60 (0.800 g) was repurified by column chromatography (silica gel, 0-10% EtOAc:DCM) to provide C60 (0.450 g, 19.3% yield) as a grey gum.

C59: (LC/MS) m/z (M+H)⁺=350.1; ¹H NMR (400 MHz, (CD₃)₂SO) δ 4.49 (s, 2H), 4.26 (q, 2H), 3.96 (d, 2H), 3.61 (t, 2H), 2.73 (t, 2H), 1.44-1.40 (m, 9H), 1.29 (t, 3H), 1.24-1.16 (m, 1H), 0.54-0.47 (m, 2H), 0.37-0.31 (m, 2H).

C60: (LC/MS) m/z (M+H)⁺=350.0; ¹H NMR (400 MHz, CDCl₃) δ 4.62 (s, 2H), 4.41-4.30 (m, 4H), 3.76-3.61 (m, 2H), 2.81-2.71 (m, 2H), 1.50-1.46 (m, 9H), 1.42-1.30 (m, 4H), 0.54-0.47 (m, 2H), 0.43-0.37 (m, 2H).

### Step 2. Preparation of ethyl 1-(cyclopropylmethyl)-4,5,6,7-tetrahydro-1H-pyrazolo[4,3-c]pyridine-3-carboxylate (P31)

To a solution of C59 (0.200 g, 0.572 mmol) in DCM (0.5 mL), 2M HCl in dioxane (2.0 mL) was added. The reaction mixture was stirred at 35° C. for 3 h and then concentrated in vacuo to provide P31 (0.143 g, crude) as a grey solid. The solid was used directly in the next step without further purification.

### Preparation 32

### rac-Ethyl (R)-1-(1-methoxypropan-2-yl)-4,5,6,7-tetrahydro-1H-pyrazolo[4,3-c]pyridine-3-carboxylate hydrochloride (P32)

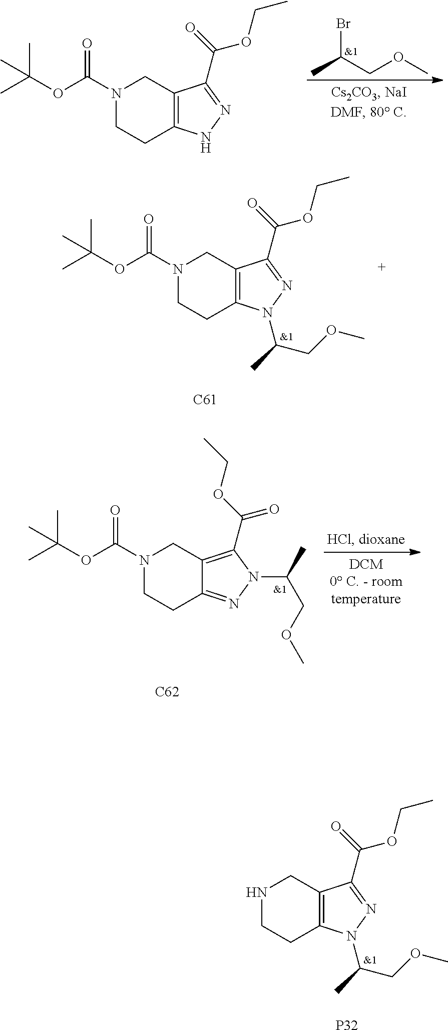

### Step 1. Preparation of rac-5-(tert-butyl) 3-ethyl (R)-1-(1-methoxypropan-2-yl)-1,4,6,7-tetrahydro-5H-pyrazolo[4,3-c]pyridine-3,5-dicarboxylate (C61) and rac-(R)-2-((3-(ethoxycarbonyl)-2-(1-methoxypropan-2-yl)-4,5,6,7-tetrahydro-2H-pyrazolo[4,3-c]pyridine-5-carbonyl)oxy)-2-methylpropan-1-ylium (C62)

The reaction was conducted in two batches then combined for purification. A solution of rac-(R)-2-bromo-1-methoxypropane (CAS: 22461-48-9; 0.829 g, 5.42 mmol), 5-(tert-butyl) 3-ethyl 1,4,6,7-tetrahydro-5H-pyrazolo[4,3-c]pyridine-3,5-dicarboxylate (CAS: 518990-23-3; 0.800 g, 2.71 mmol), Cs₂CO₃ (1.77 g, 5.42 mmol) and NaI (40.6 mg, 0.271 mmol) in DMF (13.5 mL) was stirred at 80° C. for 16 h to form the first batch.

The second batch of the same reaction was conducted with 5-(tert-butyl) 3-ethyl 1,4,6,7-tetrahydro-5H-pyrazolo[4,3-c]pyridine-3,5-dicarboxylate (CAS: 518990-23-3; 0.100 g, 0.339 mmol). The two batches were combined then diluted with H₂O (20 mL) and extracted with EtOAc (3×10 mL). The combined organic layer was dried Na₂SO₄, filtered, and concentrated in vacuo. The residue was purified by column chromatography (silica gel, 0-50% EtOAc:PE) to provide C62 (0.630 g, 56.2% yield) as an oil and C61 (0.430 g, 38.4% yield) as an oil.

C62: (LC/MS) m/z (M+H)⁺=368.2; ¹H NMR (400 MHz, CDCl₃) δ 5.72-5.60 (m, 1H), 4.68-4.55 (m, 2H), 4.33 (q, 2H), 3.85 (t, 1H), 3.74-3.63 (m, 2H), 3.55 (dd, 1H), 3.31 (s, 3H), 2.79-2.71 (m, 2H), 1.48 (s, 9H), 1.45-1.36 (m, 6H).

C61: (LC/MS) m/z (M+H)⁺=368.2; ¹H NMR (400 MHz, CDCl₃) δ 4.60 (s, 2H), 4.43-4.33 (m, 3H), 3.82-3.75 (m, 1H), 3.73-3.66 (m, 2H), 3.58 (dd, 1H), 3.24 (s, 3H), 2.83-2.62 (m, 2H), 1.52-1.46 (m, 12H), 1.38 (t, 3H).

### Step 2. Preparation of rac-ethyl (R)-1-(1-methoxypropan-2-yl)-4,5,6,7-tetrahydro-1H-pyrazolo[4,3-c]pyridine-3-carboxylate hydrochloride (P32)

At 0° C., to a suspension of C61 (0.430 g, 1.17 mmol) in DCM (2 mL) was added HCl in dioxane (8 mL) in batches. The reaction mixture was stirred at room temperature for 2 h then concentrated in vacuo to provide P32 (0.356 g, crude) as a yellow solid. The solid was used directly in the next step without further purification. (LC/MS) m/z (M+H)⁺=268.1. ¹H NMR (400 MHz, (CD₃)₂SO) δ 9.28-9.05 (m, 2H), 4.66-4.55 (m, 1H), 4.36-4.13 (m, 4H), 3.67-3.57 (m, 1H), 3.55-3.51 (m, 1H), 3.48-3.38 (m, 2H), 3.18 (s, 3H), 3.08-2.86 (m, 2H), 1.36 (d, 3H), 1.29 (t, 3H).

### Preparation 33

### Ethyl 1-(oxetan-3-yl)-4,5,6,7-tetrahydro-1H-pyrazolo[4,3-c]pyridine-3-carboxylate

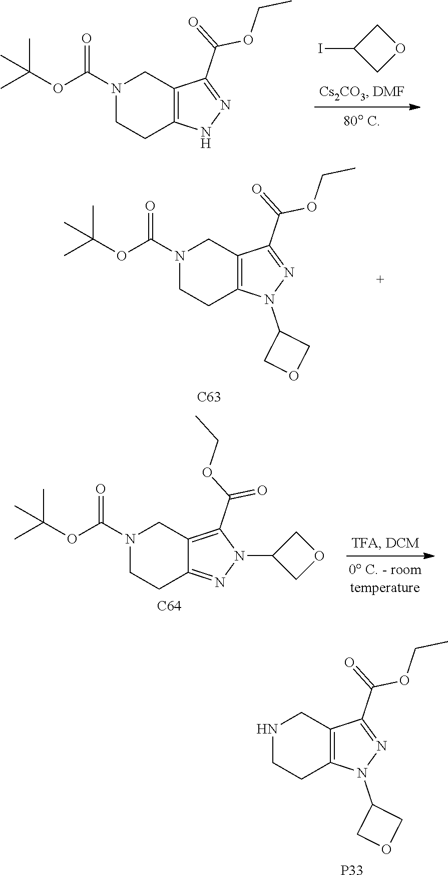

### Step 1. Preparation of 5-(tert-butyl) 3-ethyl 1-(oxetan-3-yl)-1,4,6,7-tetrahydro-5H-pyrazolo[4,3-c]pyridine-3,5-dicarboxylate (C63) and 2-((3-(ethoxycarbonyl)-2-(oxetan-3-yl)-4,5,6,7-tetrahydro-2H-pyrazolo[4,3-c]pyridine-5-carbonyl)oxy)-2-methylpropan-1-ylium (C64)

The reaction was conducted in two batches then combined for purification. To a solution of 5-(tert-butyl) 3-ethyl 1,4,6,7-tetrahydro-5H-pyrazolo[4,3-c]pyridine-3,5-dicarboxylate (CAS: 518990-23-3; 0.600 g, 2.03 mmol) in DMF (10 mL) was added 3-iodooxetane (CAS: 26272-85-5; 0.748 g, 4.06 mmol) and Cs₂CO₃ (1.32 g, 4.06 mmol) then stirred at 80° C. for 16 h to form the first batch.

The second batch of the same reaction conducted with 5-(tert-butyl) 3-ethyl 1,4,6,7-tetrahydro-5H-pyrazolo[4,3-c]pyridine-3,5-dicarboxylate (CAS: 518990-23-3; 0.100 g, 0.339 mmol) then the two batches were combined then diluted in ice H₂O (40 mL). The diluted reaction mixture was extracted with EtOAc (3×10 mL). The combined organic layer was washed with brine (2×10 mL) then concentrated in vacuo. The residue was purified by column chromatography (silica gel, 0-80% EtOAc:PE) to provide C63 (0.180 g, 25.2% yield) as an oil and C64 (0.288 g, 40.3% yield) as an oil.

C63 (Lot-001): (LC/MS) m/z (M+H)⁺=352.1; ¹H NMR (400 MHz, (CD₃)₂SO) δ 5.61-5.51 (m, 1H), 4.89 (d, 4H), 4.49 (s, 2H), 4.33-4.24 (m, 2H), 3.58 (t, 2H), 2.68 (t, 2H), 1.41 (s, 9H), 1.31 (t, 3H).

C64: (LC/MS) m/z (M-tert-butyl group)⁺=296.1; ¹H NMR (400 MHz, (CD₃)₂SO) δ 6.08-5.95 (m, 1H), 4.97-4.84 (m, 4H), 4.56-4.50 (m, 2H), 4.28 (q, 2H), 3.62 (t, 2H), 2.70 (t, 2H), 1.42 (s, 9H), 1.30 (t, 3H).

### Step 2. Preparation of ethyl 1-(oxetan-3-yl)-4,5,6,7-tetrahydro-1H-pyrazolo[4,3-c]pyridine-3-carboxylate trifluoroacetate (P33)

At 0° C., to a suspension of C63 (0.180 g, 0.512 mmol) in DCM (3 mL) was added TFA (3 mL). The reaction mixture was stirred at room temperature for 3 h and then concentrated in vacuo to provide P33 (0.187 g, crude) as a gum. The gum was used directly in the next step without further purification. (LC/MS) m/z (M+H)⁺=252.3. ¹H NMR (400 MHz, (CD₃)₂SO) δ 9.18-8.94 (m, 2H), 5.69-5.58 (m, 1H), 4.98-4.82 (m, 4H), 4.37-4.23 (m, 4H), 3.44-3.35 (m, 2H), 3.04-2.91 (m, 2H), 1.35-1.26 (m, 3H).

### Preparation 34

### Ethyl 1-(cyclobutylmethyl)-4,5,6,7-tetrahydro-1H-pyrazolo[4,3-c]pyridine-3-carboxylate hydrochloride (P34)

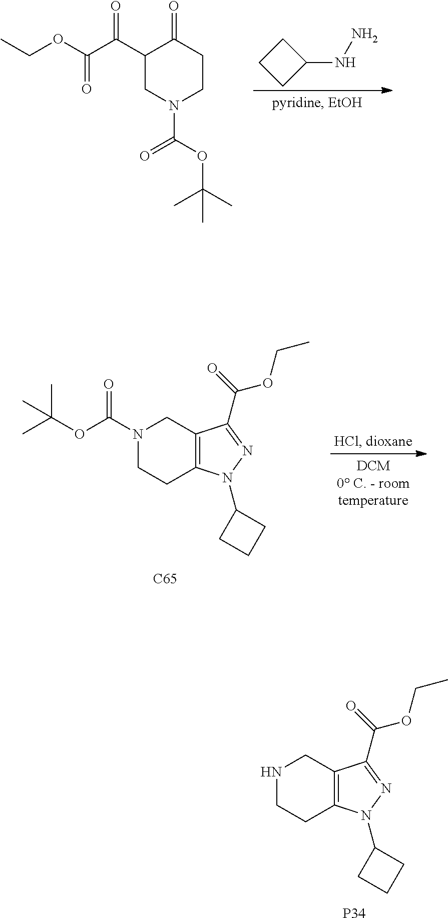

### Step 1. Preparation of 5-(tert-butyl) 3-ethyl 1-(cyclobutylmethyl)-1,4,6,7-tetrahydro-5H-pyrazolo[4,3-c]pyridine-3,5-dicarboxylate (C65)

The same procedure was followed from Preparation 2, step 1 with tert-butyl 3-(2-ethoxy-2-oxoacetyl)-4-oxopiperidine-1-carboxylate (CAS: 518990-24-4; 0.400 g, 1.34 mmol) and 1-cyclobutylhydrazine hydrochloride (CAS: 158001-21-9, 0.164 g, 1.34 mmol) to provide C65 (0.280 g, 15.0% yield) as a yellow gum. (LC/MS) m/z (M+H)⁺=350.2. ¹H NMR (400 MHz, (CD₃)₂SO) δ 4.90-4.75 (m, 1H), 4.48 (s, 2H), 4.28 (q, 2H), 3.60 (t, 2H), 2.73-2.66 (m, 2H), 2.40-2.30 (m, 4H), 1.87-1.74 (m, 2H), 1.46-1.35 (m, 9H), 1.30 (t, 3H).

### Step 2. Preparation of ethyl 1-(cyclobutylmethyl)-4,5,6,7-tetrahydro-1H-pyrazolo[4,3-c]pyridine-3-carboxylate hydrochloride (P34)

At 0° C., HCl was added to a suspension of C65 (0.280 g, 0.801 mmol) in DCM (5 mL) in dioxane (10 mL). The reaction mixture was stirred at room temperature for 3 h, concentrated in vacuo, and then lyophilized to provide P34 (0.150 g, crude) as a brown solid. The solid was used directly in the next step without further purification. (LC/MS) m/z (M+H)⁺=250.1. ¹H NMR (400 MHz, (CD₃)₂SO) δ 9.37 (s, 2H), 4.94-4.80 (m, 1H), 4.29 (q, 2H), 4.21 (s, 2H), 3.42-3.36 (m, 2H), 2.95 (t, 2H), 2.48-2.44 (m, 1H), 2.41-2.31 (m, 2H), 1.87-1.75 (m, 2H), 1.30 (t, 3H).

### Preparation 35

### rac-Ethyl (R)-1-(1,1,1-trifluoropropan-2-yl)-4,5,6,7-tetrahydro-1H-pyrazolo[4,3-c]pyridine-3-carboxylate (P35)

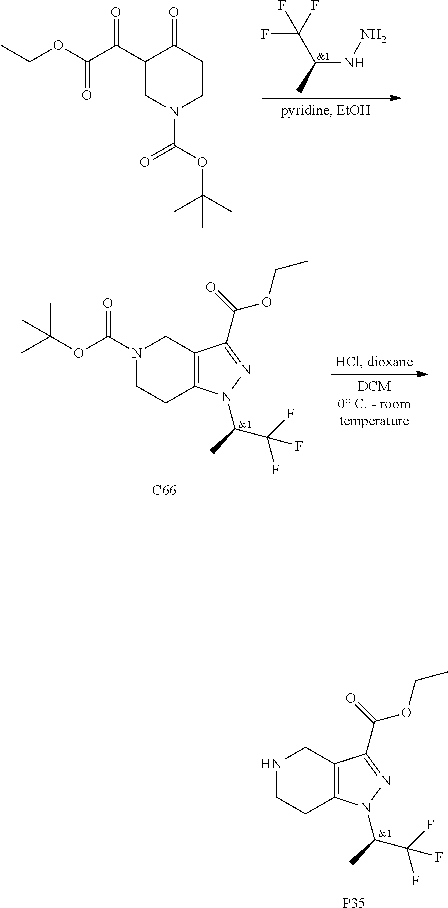

### Step 1. Preparation of rac-5-(tert-butyl) 3-ethyl (R)-1-(1,1,1-trifluoropropan-2-yl)-1,4,6,7-tetrahydro-5H-pyrazolo[4,3-c]pyridine-3,5-dicarboxylate (C66)

To a solution of tert-butyl 3-(2-ethoxy-2-oxoacetyl)-4-oxopiperidine-1-carboxylate (CAS: 518990-24-4; 0.400 g, 1.34 mmol) and (1,1,1-trifluoropropan-2-yl)hydrazine hydrochloride (CAS: 1453472-98-4; 0.269 g, 1.34 mmol) in EtOH (7 mL) was added pyridine (0.465 g, 5.88 mmol). The reaction mixture was stirred at room temperature for 5 h and then concentrated in vacuo. The residue was purified by column chromatography (silica gel, 0-25% EtOAc:PE) to provide C66 (0.415 g, 79.3% yield) as a yellow gum. (LC/MS) m/z (M+H)⁺=392.2. ¹H NMR (400 MHz, (CD₃)₂SO) δ 5.47-5.35 (m, 1H), 4.60-4.38 (m, 2H), 4.29 (q, 2H), 3.78-3.67 (m, 1H), 3.55-3.46 (m, 1H), 2.92-2.81 (m, 1H), 2.69-2.56 (m, 1H), 1.67 (d, 3H), 1.41 (s, 9H), 1.30 (t, 3H).

### Step 2. Preparation of rac-ethyl (R)-1-(1,1,1-trifluoropropan-2-yl)-4,5,6,7-tetrahydro-1H-pyrazolo[4,3-c]pyridine-3-carboxylate (P35)

At 0° C., to a suspension of C66 (0.415 g, 1.06 mmol) in DCM (4 mL) was added HCl in dioxane (8 mL) in batches. The reaction mixture was stirred at room temperature for 16 h then concentrated in vacuo to provide P35 (0.306 g, crude) as a yellow solid. The solid was used directly in the next step without further purification.

### Preparation 36

### Ethyl 1,7,7-trimethyl-4,5,6,7-tetrahydro-1H-pyrazolo[4,3-c]pyridine-3-carboxylate hydrochloride (P36)

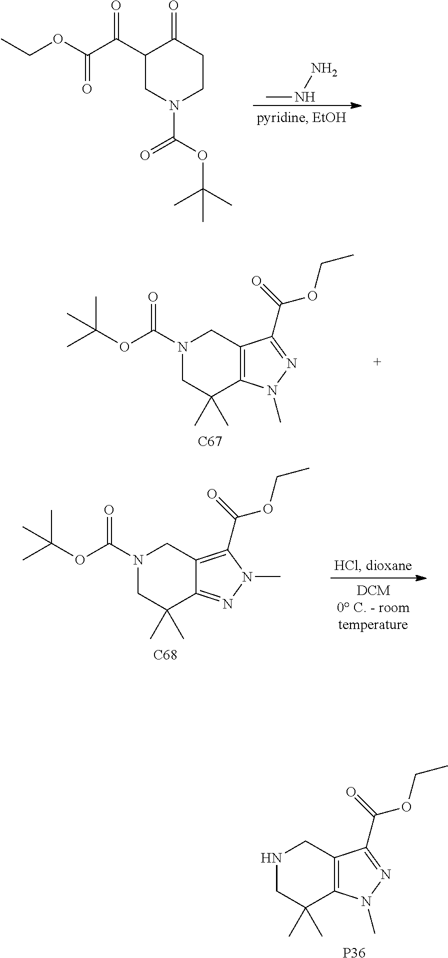

### Step 1. Preparation of 5-(tert-butyl) 3-ethyl 1,7,7-trimethyl-1,4,6,7-tetrahydro-5H-pyrazolo[4,3-c]pyridine-3,5-dicarboxylate (C67) and 2-((3-(ethoxycarbonyl)-2,7,7-trimethyl-4,5,6,7-tetrahydro-2H-pyrazolo[4,3-c]pyridine-5-carbonyl)oxy)-2-methylpropan-1-ylium (C68)

The same procedure was followed from Preparation 2, step 1 with tert-butyl 5-(2-ethoxy-2-oxoacetyl)-3,3-dimethyl-4-oxopiperidine-1-carboxylate (CAS: 2171295-98-8, 1.28 g, 3.91 mmol) and methylhydrazine (CAS: 60-34-4, 1.08 g, 9.38 mmol) to provide C68 (0.563 g, 42.7% yield) as a light-yellow gum and C67 (0.160 g, 12.1% yield) as a light-yellow gum.

C67: (LC/MS) m/z (M+H)⁺=338.2; ¹H NMR (400 MHz, CDCl₃) δ 4.66-4.54 (m, 2H), 4.44-4.30 (m, 2H), 4.00-3.92 (m, 3H), 3.45-3.34 (m, 2H), 1.51-1.45 (m, 9H), 1.41-1.35 (m, 3H), 1.34-1.29 (m, 6H).

C68: (LC/MS) m/z (M+H)⁺=338.1; ¹H NMR (400 MHz, CDCl₃) δ 4.66-4.56 (m, 2H), 4.38-4.26 (m, 2H), 4.16-4.05 (m, 3H), 3.45-3.32 (m, 2H), 1.49-1.46 (m, 9H), 1.37 (t, 3H), 1.28-1.24 (m, 6H).

### Step 2. Preparation of ethyl 1,7,7-trimethyl-4,5,6,7-tetrahydro-1H-pyrazolo[4,3-c]pyridine-3-carboxylate hydrochloride (P36)

At 0° C., to a suspension of C67 (0.130 g, 0.385 mmol) in DCM (3 mL) was added HCl in dioxane (3 mL). The reaction mixture was stirred at room temperature for 2 h and then concentrated in vacuo to provide P36 (0.110 g, crude) as a yellow solid. The solid was used directly in the next step without further purification. (LC/MS) m/z (M+H)⁺=238.1. ¹H NMR (400 MHz, (CD₃)₂SO) δ 9.58 (s, 2H), 4.27 (q, 2H), 4.18 (s, 2H), 3.98 (s, 3H), 3.21-3.16 (m, 2H), 1.42 (s, 6H), 1.31-1.22 (m, 3H).

### Preparation 37

### Ethyl 1′-methyl-1,4′,5′,6′-tetrahydrospiro[cyclopropane-1,7′-pyrazolo[4,3-c]pyridine]-3′-carboxylate hydrochloride (P37)

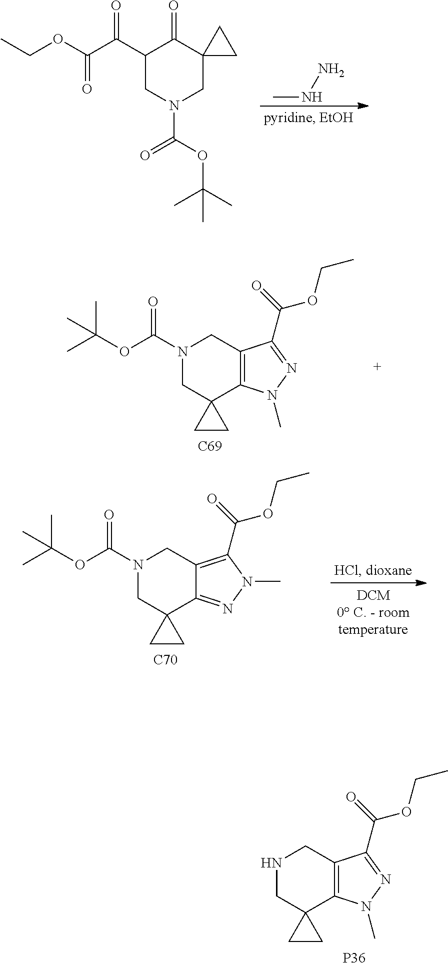

### Step 1. Preparation of 5′-(tert-butyl) 3′-ethyl 1′-methyl-1′,4′-dihydrospiro[cyclopropane-1,7′-pyrazolo[4,3-c]pyridine]-3′,5′(6′H)-dicarboxylate (C69) and 2-((3′-(ethoxycarbonyl)-2′-methyl-2′,4′,5′,6′-tetrahydrospiro[cyclopropane-1,7′-pyrazolo[4,3-c]pyridine]-5′-carbonyl)oxy)-2-methylpropan-1-ylium (C70)

The same procedure was followed from Preparation 2, step 1 with tert-butyl 7-(2-ethoxy-2-oxoacetyl)-8-oxo-5-azaspiro[2.5]octane-5-carboxylate (CAS: 2494304-89-9; 0.620 g, 1.91 mmol) and methylhydrazine sulfate (CAS: 302-15-8, 0.302 g, 2.10 mmol). The purification was altered. The residue was purified by column chromatography (silica gel, 0-30% THF:PE) to provide C70 (0.327 g, 51.2% yield) as a white solid and C69 (0.290 g, 45.4% yield) as a white gum.

C69: (LC/MS) m/z (M+H)⁺=336.3; ¹H NMR (400 MHz, CDCl₃) δ 4.70 (s, 2H), 4.39 (q, 2H), 3.74 (s, 3H), 3.44 (s, 2H), 1.48 (s, 9H), 1.39 (t, 3H), 1.31 (t, 2H), 1.08-0.94 (m, 2H).

C70: (LC/MS) m/z (M+H)⁺=336.3; ¹H NMR (400 MHz, CDCl₃) δ 4.75-4.65 (m, 2H), 4.34 (q, 2H), 4.08 (s, 3H), 3.53-3.47 (m, 2H), 1.48 (s, 9H), 1.39 (t, 3H), 1.12 (q, 2H), 0.97-0.86 (m, 2H).

### Step 2. Preparation of ethyl 1′-methyl-1,4′,5′,6′-tetrahydrospiro[cyclopropane-1,7′-pyrazolo[4,3-c]pyridine]-3′-carboxylate hydrochloride (P37)

At 0° C., to a suspension of C69 (0.272 g, 0.811 mmol) in DCM (6 mL) was added HCl in dioxane (0.591 g, 16.2 mmol). The reaction mixture was stirred at room temperature for 3 h then concentrated in vacuo to provide P37 (0.219 g, crude) as a white solid. The solid was used directly in the next step without further purification.

### Preparation 38

### rac-Ethyl (R)-1-isopropyl-7-methyl-4,5,6,7-tetrahydro-1H-pyrazolo[4,3-c]pyridine-3-carboxylate hydrochloride (P38)

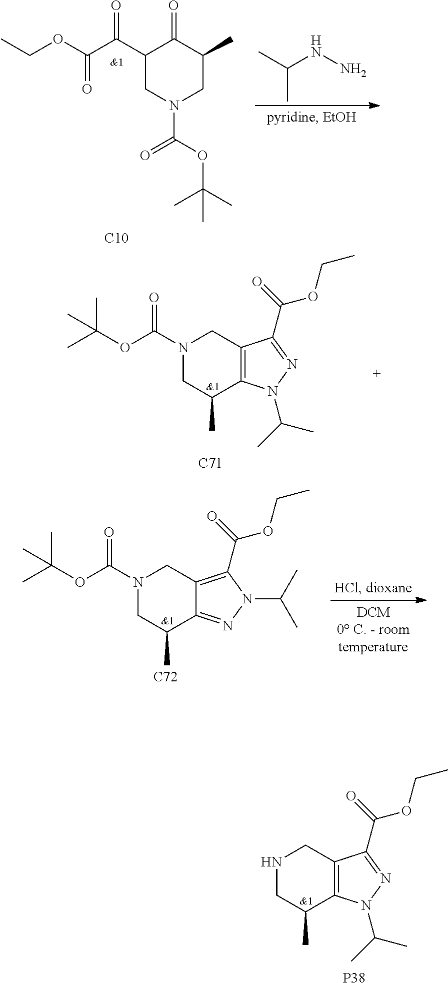

### Step 1. Preparation of rac-5-(tert-butyl) 3-ethyl (R)-1-isopropyl-7-methyl-1,4,6,7-tetrahydro-5H-pyrazolo[4,3-c]pyridine-3,5-dicarboxylate (C71) and rac-5-(tert-butyl) 3-ethyl (R)-2-isopropyl-7-methyl-2,4,6,7-tetrahydro-5H-pyrazolo[4,3-c]pyridine-3,5-dicarboxylate (C72)

The same procedure was followed from Preparation 2, step 1 with C10 (3.00 g, 9.57 mmol) and isopropylhydrazine hydrochloride (CAS: 16726-41-3, 1.06 g, 9.57 mmol). The purification was altered. The residue was purified by reverse phase HPLC (Sunfire C₁₈ 150 mm×40 mm×5 μm column, Mobile Phase A: H₂O (0.05% NH₄OH—NH₄HCO₃)/Mobile Phase B: ACN, Mobile Phase B: 42 to 82% over 9.0 min, 100% of Mobile Phase B hold 2.0 min, flow rate=30 mL/min) to provide C71 (1.80 g, 53.5% yield) as a pale yellow solid and C72 (33.9 mg, 1.01% yield) as a pale yellow solid.

C71: (LC/MS) m/z (M+H)⁺=352.4. ¹H NMR (400 MHz, CDCl₃) δ 5.05 (dd, 1H), 4.46-4.31 (m, 3H), 4.13 (dd, 2H), 3.20-3.04 (m, 1H), 2.99-2.89 (m, 1H), 1.54 (d, 3H), 1.51-1.45 (m, 12H), 1.36 (t, 3H), 1.23 (d, 3H).

C72: (LC/MS) m/z (M+H)⁺=352.4. ¹H NMR (400 MHz, CDCl₃) δ 5.56-5.44 (m, 1H), 4.69-4.45 (m, 2H), 4.32 (q, 2H), 3.90-3.61 (m, 1H), 3.41-3.13 (m, 1H), 3.02-2.92 (m, 1H), 1.51-1.44 (m, 15H), 1.38 (t, 3H), 1.26 (d, 3H).

### Step 2. Preparation of rac-ethyl (R)-1-isopropyl-7-methyl-4,5,6,7-tetrahydro-1H-pyrazolo[4,3-c]pyridine-3-carboxylate hydrochloride (P38)

At 0° C., to a suspension of C71 (0.200 g, 0.569 mmol) in DCM (1 mL) was added HCl in dioxane (4 mL). The reaction mixture was stirred at room temperature for 2 h then concentrated in vacuo to provide P38 (0.180 g, crude) as a yellow solid. The solid was used directly in the next step without further purification. (LC/MS) m/z (M+H)⁺=252.1. ¹H NMR (400 MHz, (CD₃)₂SO) δ 9.98-9.83 (m, 1H), 9.34-9.25 (m, 1H), 4.67-4.54 (m, 1H), 4.33-4.05 (m, 4H), 3.43-3.26 (m, 3H), 1.45-1.40 (m, 3H), 1.39-1.33 (m, 6H), 1.32-1.26 (m, 3H).

### Preparation 39

### 2-Fluoro-5-(1-methyl-1H-1,2,4-triazol-5-yl)pyridine (P39)

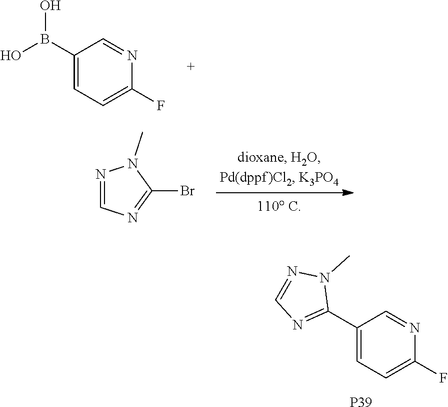

### Step 1. Preparation of 2-fluoro-5-(1-methyl-1H-1,2,4-triazol-5-yl)pyridine (P39)

To a suspension of (6-fluoropyridin-3-yl)boronic acid (CAS: 351019-18-6; 1.10 g, 7.81 mmol) and 5-bromo-1-methyl-1H-1,2,4-triazole (CAS: 16681-72-4; 1.52 g, 9.37 mmol) in dioxane (10 mL) and H₂O (2 mL) was added Pd(dppf)Cl₂ (0.457 g, 0.625 mmol) and K₃PO₄ (4.97 g, 23.4 mmol). The reaction mixture was degassed with nitrogen gas, then stirred at 110° C. for 4 h and purified by column chromatography (silica gel, 0-40% EtOAc:PE) to provide P39 (1.20 g, 86.3% yield) as a white solid. (LC/MS) m/z (M+H)⁺=179.1. ¹H NMR (400 MHz, (CD₃)₂SO) δ 8.67-8.62 (m, 1H), 8.42-8.34 (m, 1H), 8.05 (s, 1H), 7.40 (dd, 1H), 3.97 (s, 3H).

### Preparation 40

### 2-Fluoro-5-(1-methyl-1H-imidazol-2-yl)pyridine (P40)

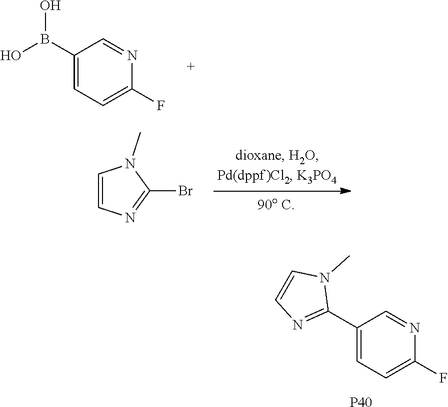

### Step 1. Preparation of 2-fluoro-5-(1-methyl-1H-imidazol-2-yl)pyridine (P40)

Under nitrogen gas, the reaction mixture of 2-bromo-1-methyl-1H-imidazole (0.700 g, 4.35 mmol), (6-fluoropyridin-3-yl)boronic acid (1.20 g, 8.70 mmol) and Pd(dppf)Cl₂ (0.178 g, 0.217 mmol) suspended in dioxane (10 mL) and H₂O (2 mL) was stirred at 90° C. for 1 h. The reaction mixture was extracted with EtOAc, and then the organic layer was concentrated in vacuo. The residue was dissolved in DCM and then purified by column chromatography (silica gel, 0-10% MeOH:DCM) to provide P40 (0.688 g, 89.3% yield) as an orange oil. (LC/MS) m/z (M+H)⁺=178.1.

### Preparation 41

### 2-(4-(Aminomethyl)phenyl)pyrimidin-4(3H)-one hydrochloride (P41)

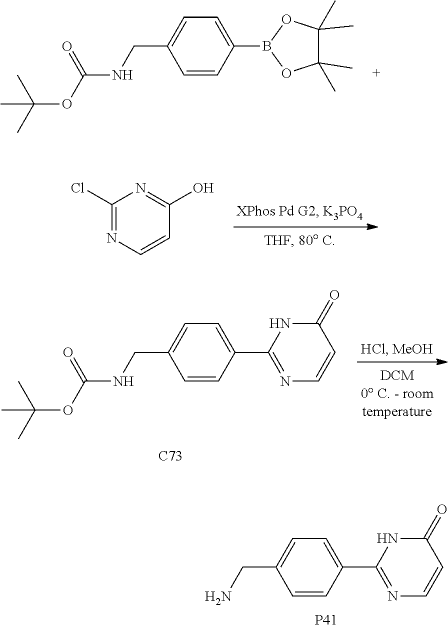

### Step 1. Preparation of tert-butyl (4-(6-oxo-1,6-dihydropyrimidin-2-yl)benzyl)carbamate (C73)

To a solution of 2-chloropyrimidin-4-ol (CAS: 55873-09-1; 0.100 g, 0.766 mmol) and tert-butyl (4-(4,4,5,5-tetramethyl-1,3,2-dioxaborolan-2-yl)benzyl)carbamate (CAS: 330794-35-9; 0.255 g, 0.766 mmol) in THF (7.7 mL) was added 2M K₃PO₄ (0.325 g, 1.53 mmol). The reaction mixture was degassed with nitrogen gas for 3 min, and then XPhos Pd G2 (60.3 mg, 0.0766 mmol) was added. The suspension was degassed with nitrogen gas for 3 min and then stirred at 80° C. for 4 h. The reaction solution was diluted with H₂O (10 mL) and then extracted with MeOH:DCM (1:10, 3×5 mL). The combined organic layer was dried with Na₂SO₄, filtered, and the filtrate was concentrated in vacuo. The residue was purified by column chromatography (silica gel, 0-100% EtOAc:PE) to provide C73 (0.115 g, 49.8% yield) as a white solid. (LC/MS) m/z (M+H)⁺=302.1. ¹H NMR (400 MHz, (CD₃)₂SO) δ 12.75 (br s, 1H), 8.16-7.92 (m, 3H), 7.51-7.44 (m, 1H), 7.36 (d, 2H), 6.36-6.24 (m, 1H), 4.18 (d, 2H), 1.39 (s, 9H).

### Step 2. Preparation of 2-(4-(aminomethyl)phenyl)pyrimidin-4(3H)-one hydrochloride (P41)

At 0° C., to a stirred solution of C73 (0.115 g, 0.382 mmol) in DCM (1 mL) was added HCl in MeOH (3 mL). The reaction mixture was stirred at room temperature for 2 h and then concentrated in vacuo to provide P41 (91.0 mg, crude) as a white solid. The solid was used directly in the next step without further purification. ¹H NMR (400 MHz, (CD₃)₂SO) δ 11.05-10.97 (m, 1H), 10.88-10.81 (m, 1H), 8.60-8.47 (m, 2H), 8.17-8.08 (m, 2H), 7.66-7.61 (m, 2H), 7.39 (dd, 1H), 6.40-6.36 (m, 1H), 5.46-5.42 (m, 1H), 4.10 (q, 2H).

### Preparation 42

### 6-(4-(Aminomethyl)phenyl)pyridin-2(1H)-one hydrochloride (P42)

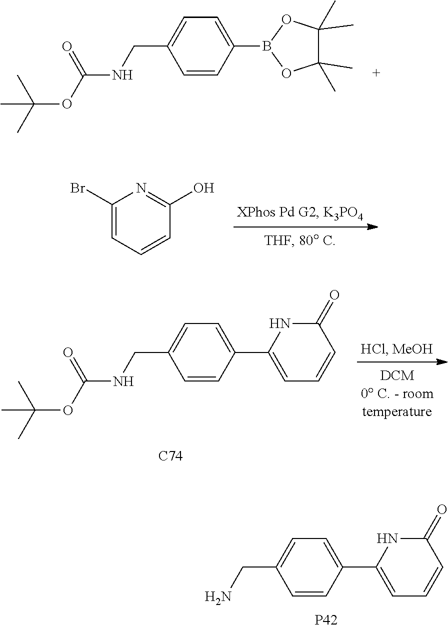

### Step 1. Preparation of tert-butyl (4-(6-oxo-1,6-dihydropyridin-2-yl)benzyl)carbamate (C74)

To a solution of 6-bromopyridin-2-ol (CAS: 27992-32-1; 0.100 g, 0.575 mmol) and tert-butyl (4-(4,4,5,5-tetramethyl-1,3,2-dioxaborolan-2-yl)benzyl)carbamate (CAS: 330794-35-9; 0.192 g, 0.575 mmol) in THF (5.7 mL) was added 2M K₃PO₄ (0.244 g, 1.15 mmol). The reaction mixture was degassed with nitrogen gas for 3 min, then XPhos Pd G2 (45.2 mg, 0.0575 mmol) was added. The suspension was degassed with nitrogen gas for 3 min and then stirred at 80° C. for 4 h. The reaction solution was diluted with H₂O (10 mL) and then extracted with EtOAc (3×5 mL). The combined organic layer was dried with Na₂SO₄, filtered, and the filtrate was concentrated in vacuo. The residue was purified by column chromatography (silica gel, 0-100% EtOAc:PE) to provide C74 (0.110 g, 63.7% yield) as a white solid. (LC/MS) m/z (M+H)⁺=301.1. ¹H NMR (400 MHz, (CD₃)₂SO) δ 11.71 (brs, 1H), 7.73 (d, 2H), 7.58-7.51 (m, 1H), 7.49-7.44 (m, 1H), 7.33 (d, 2H), 6.71-6.57 (m, 1H), 6.37 (d, 1H), 4.18 (d, 2H), 1.41 (s, 9H).

### Step 2. Preparation of 6-(4-(aminomethyl)phenyl)pyridin-2(1F)-one hydrochloride (P42)

At 0° C., to a stirred solution of C74 (0.110 g, 0.366 mmol) in DCM (1 mL) was added HCl in MeOH (3 mL). The reaction mixture was stirred at room temperature for 2 h and then concentrated in vacuo to provide P42 (87.0 mg, crude) as a white solid. The solid was used directly in the next step without further purification. ¹H NMR (400 MHz, (CD₃)₂SO) δ 8.46-8.36 (m, 3H), 7.84 (d, 2H), 7.61-7.54 (m, 3H), 6.80-6.70 (m, 1H), 6.43 (d, 1H), 4.07 (q, 2H).

### Preparation 43

### 3-(4-(Aminomethyl)phenyl-2,3,5,6-d₄)-1,1-dimethylurea hydrochloride (P43)

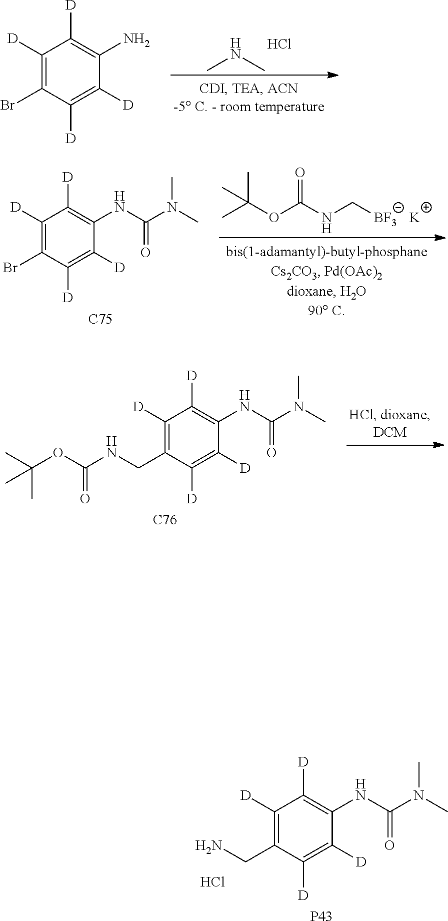

### Step 1. Preparation of 3-(4-bromophenyl-2,3,5,6-d₄)-1,1-dimethylurea (C75)

To a solution of CDI (6.59 g, 40.6 mmol) in ACN (50 mL) was added 4-bromobenzen-2,3,5,6-d₄-amine (CAS: 61357-76-4; 5.50 g, 31.2 mmol) at (−5 to 5) ° C. The reaction mixture was stirred from (0 to 5) ° C. for 3 h before TEA (6.32 g, 62.5 mmol) and dimethylamine hydrochloride (CAS: 506-59-2; 5.10 g, 62.5 mmol) were added slowly at (0 to 5) ° C. The reaction mixture was warmed to room temperature and stirred for 16 h. The suspension was poured into H₂O (100 mL) and extracted with EtOAc (3×100 mL). The combined organic layer was washed with brine (200 mL), dried over MgSO4, and concentrated in vacuo. The residue was slurried with MTBE:DCM (10:1, 100 mL) at room temperature for 3 h and then filtered. The filter cake was dried in vacuo and then collected to provide C75 (6.10 g, 76.2% yield) as a white solid. ¹H NMR (400 MHz, (CD₃)₂SO) δ 8.40 (s, 1H), 2.91 (s, 6H).

### Step 2. Preparation of tert-butyl (4-(3,3-dimethylureido)2,3,5,6-d₄-benzyl)carbamate (C76)

A reaction mixture of C75 (3.0 g, 12 mmol), potassium [[(tert-butoxycarbonyl)amino]methyl]trifluoroborate (4.3 g, 18 mmol) and Cs₂CO₃ (7.9 g, 24 mmol) in H₂O (6 mL) and dioxane (40 mL) was degassed and purged with nitrogen gas 3 times. To the reaction mixture were added bis(1-adamantyl)-butyl-phosphane (0.87 g, 2.4 mmol) and Pd(OAc)₂ (0.27 g, 1.2 mmol). The suspension was degassed and purged with nitrogen gas 3 times again, then stirred at 90° C. for 16 h under nitrogen gas. The reaction mixture was cooled to room temperature and then poured into H₂O (50 mL) and filtered. The filtrate was extracted with EtOAc (3×50 mL). The combined organic layer was washed with brine (100 mL), dried over MgSO4, and concentrated in vacuo. The residue was slurried with DCM (100 mL) at room temperature for 4 h and then filtered. The filter cake was dried in vacuo and then collected to provide C76 (2.0 g, 30.% yield) as a light-yellow solid.

### Step 3. Preparation of 3-(4-(aminomethyl)phenyl-2,3,5,6-d₄)-1,1-dimethylurea hydrochloride (P43)

To a solution of C76 (2.0 g, 6.7 mmol) in DCM (30 mL) was added 2M HCl in dioxane (25 mL). The reaction mixture was stirred at room temperature for 16 h and then concentrated in vacuo. The residue was purified by reverse phase HPLC (Welch Xtimate C₁₈ 250 mm×50 mm×10 μm column, Mobile Phase A: H₂O with HCl/Mobile Phase B: ACN, 0 to 20% of Mobile Phase B over 20 min) then lyophilized to provide P43 (1.1 g, 67% yield) as a white solid. ¹H NMR (400 MHz, (CD₃)₂SO) δ 8.43 (s, 1H), 8.37 (br s, 2H), 3.90 (q, 2H), 2.92 (s, 6H).

### Preparation 44

### rac-Ethyl (R)-1-isopropyl-7-(trifluoromethyl)-4,5,6,7-tetrahydro-1H-pyrazolo[4,3-c]pyridine-3-carboxylate hydrochloride (P44)

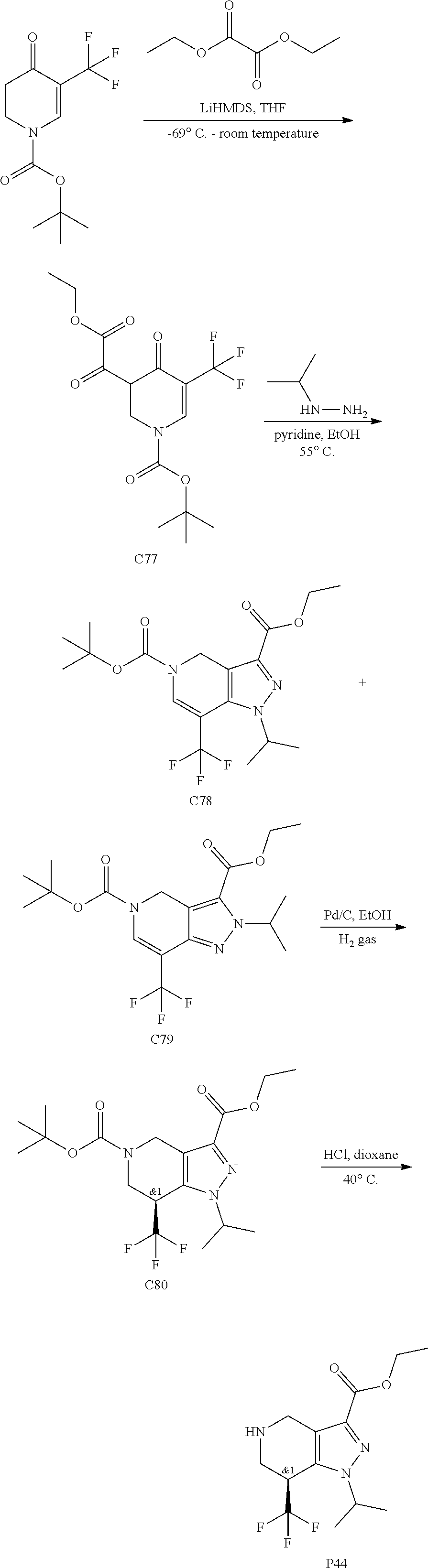

### Step 1. Preparation of tert-butyl 3-(2-ethoxy-2-oxoacetyl)-4-oxo-5-(trifluoromethyl)-3,4-dihydropyridine-1(2H)-carboxylate (C77)

A yellow solution of 1M LiHMDS in THF (18.4 mL) was diluted with THF (10 mL) then cooled to −69° C. with a dry ice/acetone bath. To the cooled solution was added tert-butyl 4-oxo-5-(trifluoromethyl)-3,4-dihydropyridine-1(2H)-carboxylate (CAS: 1667744-92-4; 4.07 g, 15.4 mmol) in THF (20 mL) dropwise over 15 min. The substrate flask was rinsed with THF (2×1 mL) and added to the reaction mixture. During the addition, the solution became a brownish-orange color. The reaction was stirred at −69° C. for 45 min after the addition.

A solution of diethyl oxalate (2.52 mL) in THF (5 mL) was added dropwise to the reaction mixture over 8 min. The source vial was rinsed with THF (1.5 mL), which was then added to the reaction mixture, which changed the solution to a brownish-orange color. The reaction mixture was stirred in the dry ice/acetone bath for another 7 min, then removed and replaced with an ice water bath. The reaction mixture was quenched with aqueous 2N HCl (21 mL), which changed the color of the suspension to an orange color. The suspension was diluted with H₂O and extracted with EtOAc (2×15 mL). The combined organic layers were washed with saturated NaHCO₃ solution (1×20 mL, 2×10 mL). The organic layers were set aside. The aqueous layers were combined and washed with MTBE (3×20 mL). The combined organic layers were set aside. The aqueous layer was acidified with 6N HCl (˜6.5 mL) to pH=1. The acidic aqueous layer was extracted with MTBE (2×40 mL). The combined organic layer from the acidic aqueous layer extraction was dried with Na₂SO₄, filtered, and concentrated in vacuo, then dried further by high vacuum to provide C77 (1.52 g) as an orange-yellow residue.

The organic layers that were set aside were combined. The combined organic layer was dried with Na₂SO₄, filtered, and concentrated in vacuo to give a deep orange oil. The oil was dissolved in MTBE (40 mL) and then washed with saturated NaHCO₃ solution (1×32 mL, 1×10 mL). The aqueous layers were combined and acidified with aqueous 6N HCl. The acidic aqueous layer was extracted with MTBE (30 mL) and then MTBE (20 mL). The organic layers were combined and then dried with Na₂SO₄, filtered, concentrated in vacuo, and dried further by high vacuum to provide C77 (1.92 g) as an orange residue.

The two batches of products were combined to form C77 (3.44 g) and then purified by column chromatography (silica gel, 0-85% EtOAc:heptane) to provide C77 (2.32 g, 41.3% yield) as a yellow oil. (LC/MS) m/z (M+H)⁺=366.5. 1H NMR (400 MHz, CDCl₃) δ 15.18 (s, 1H), 8.28 (s, 1H), 5.03 (s, 2H), 4.38 (q, 2H), 1.58 (s, 9H), 1.40 (t, 3H).

### Step 2. Preparation of 5-(tert-butyl) 3-ethyl 1-isopropyl-7-(trifluoromethyl)-1,4,6,7-tetrahydro-5H-pyrazolo[4,3-c]pyridine-3,5-dicarboxylate (C78) and 2-((3-(ethoxycarbonyl)-2-isopropyl-7-(trifluoromethyl)-4,5,6,7-tetrahydro-2H-pyrazolo[4,3-c]pyridine-5-carbonyl)oxy)-2-methylpropan-1-ylium (C79)

To a solution of C77 (408 mg, 1.12 mmol) in EtOH (4.5 mL) was added isopropylhydrazine hydrochloride (CAS: 16726-41-3; 148 mg, 1.34 mmol) and then pyridine (132 mg, 1.67 mmol). The reaction mixture was stirred at 55° C. for 20 h and then concentrated in vacuo to give a yellow-orange residue. The residue was diluted with EtOAc (20 mL) and washed with NaHCO₃ (20 mL). The aqueous layer was back-extracted with EtOAc (2×10 mL). The combined organic layers were washed with brine, dried with Na₂SO₄, filtered, and concentrated in vacuo to give an orange oil. The oil was dissolved in DCM then preabsorbed on (1:1) silica gel: Celite and purified by column chromatography (silica gel, 0-100% EtOAc:heptane) to provide C78 (0.315 g, 69.9% yield) as a gum and C79 (7.70 mg, 1.71% yield) as a light-yellow solid.

C78: (LC/MS) m/z (M+H)⁺=404.6; ¹H NMR (400 MHz, CDCl₃) δ 7.67-7.46 (m, 1H), 5.02 (s, 2H), 4.73-4.63 (m, 1H), 4.39 (q, 2H), 1.55 (s, 9H), 1.50 (d, 6H), 1.39 (t, 3H).

C79: (LC/MS) m/z (M+H)⁺=404.5; ¹H NMR (500 MHz, CDCl₃) δ 7.63-7.33 (m, 1H), 5.51-5.49 (m, 1H), 4.97 (s, 2H), 4.35 (q, 2H), 1.55 (s, 9H), 1.48 (d, 6H), 1.39 (t, 3H).

### Step 3. Preparation of rac-5-(tert-butyl) 3-ethyl (R)-1-isopropyl-7-(trifluoromethyl)-1,4,6,7-tetrahydro-5H-pyrazolo[4,3-c]pyridine-3,5-dicarboxylate (C80)

To a suspension of Pd/C (CAS: 7440-05-3; 83 mg, 5% wt, 0.039 mmol) in EtOH (0.5 mL) was added a light-yellow solution of C78 (0.30 g, 0.75 mmol) in EtOH (1.5 mL). The source vial was rinsed with EtOH (0.5 mL×2), and then the rinses were added to the reaction mixture for a total volume of EtOH (3 mL). The reaction mixture was stirred at room temperature overnight in a Hastelloy reactor at H₂ gas (50 psi). The suspension was filtered through Celite (0.5 g) with a prepacked plastic filter funnel. The filter cake was eluted with EtOH. The grey filtrate was concentrated in vacuo to give a grey residue.

The same reaction was set up again. To a reaction mixture of Pd/C (98 mg, 5 wt %, 0.046 mmol) in EtOH (0.5 mL) was added the solution of the crude residue dissolved in EtOH (1.5 mL). The reaction mixture was stirred at room temperature overnight in a Hastelloy reactor at H₂ gas (50 psi). The suspension was filtered through Celite (0.5 g) with a prepacked plastic filter funnel. The filter cake was eluted with EtOH. The grey filtrate was concentrated in vacuo to provide a grey solid. The solid was purified by column chromatography (silica gel, 0-50% EtOAc:heptane) to provide C80 (0.24 g, 78% yield) as a white solid. (LC/MS) m/z (M+H)⁺=406.6; ¹H NMR (400 MHz, CDCl₃) δ 5.35-5.23 (m, 1H), 5.13-4.76 (m, 2H), 4.59-4.47 (m, 1H), 4.44-4.34 (m, 3H), 4.29-4.08 (m, 2H), 3.60-3.44 (m, 1H), 3.08 (dd, 1H), 1.59-1.55 (m, 9H), 1.43-1.35 (m, 6H).

### Step 4. Preparation of rac-ethyl (R)-1-isopropyl-7-(trifluoromethyl)-4,5,6,7-tetrahydro-1H-pyrazolo[4,3-c]pyridine-3-carboxylate hydrochloride (P44)

To C80 (0.234 g, 0.577 mmol) was added 4N HCl in dioxane (1.44 mL) dropwise. The reaction mixture was stirred at 40° C. for 40 min then concentrated under nitrogen gas then dried further under high vacuum to provide P44 (0.210 g, crude) as a white solid. (LC/MS) m/z (M+H)⁺=306.5; ¹H NMR (400 MHz, (CD₃)₂SO) δ 10.41 (br s, 1H), 8.55 (br s, 1H), 4.91-4.80 (m, 1H), 4.76-4.65 (m, 1H), 4.40-4.20 (m, 3H), 3.92 (d, 1H), 3.74-3.64 (m, 1H), 3.55-3.45 (m, 1H), 1.44 (d, 3H), 1.36 (d, 3H), 1.30 (t, 3H).

### Preparation 45

### 1-(6-Fluoropyridin-3-yl)pyrrolidin-2-one (P45)

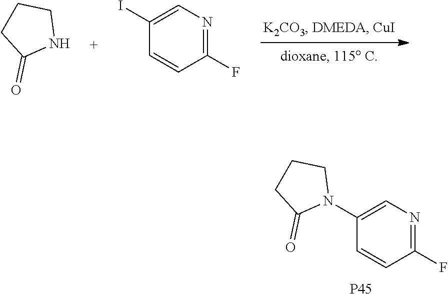 [SMILES: O=C1CCCN1 ; Fc1ccc(I)cn1 ; "P45" ; O=C1CCCN1c1ccc(F)nc1]

### Step 1. Preparation of 1-(6-fluoropyridin-3-yl)pyrrolidin-2-one (P45)

A reaction mixture of 2-fluoro-5-iodopyridine (CAS: 171197-80-1; 2.00 g, 8.97 mmol), pyrrolidin-2-one (CAS: 616-45-5; 0.763 g, 8.97 mmol), K₂CO₃ (3.72 g, 26.9 mmol), DMEDA (0.158 g, 1.79 mmol) and CuI (0.342 g, 1.79 mmol) in dioxane (40 mL) was heated to 115° C. for 16 h under nitrogen gas. The suspension was filtered, and the filtrate was concentrated in vacuo. The residue was purified by column chromatography (silica gel, 0-60% EtOAc:PE) to provide P45 (1.40 g, 86.6% yield) as a light-yellow solid. (LC/MS) m/z (M+H)⁺=181.0; ¹H NMR (400 MHz, (CD₃)₂SO) δ 8.46-8.43 (m, 1H), 8.36-8.29 (m, 1H), 7.22 (dd, 1H), 3.89-3.83 (m, 2H), 2.54-2.51 (m, 1H), 2.50-2.48 (m, 1H), 2.14-2.04 (m, 2H).

### Preparation 46

### Ethyl (R)-1-ethyl-6-methyl-4,5,6,7-tetrahydro-1H-pyrazolo[4,3-c]pyridine-3-carboxylate hydrochloride (P46)

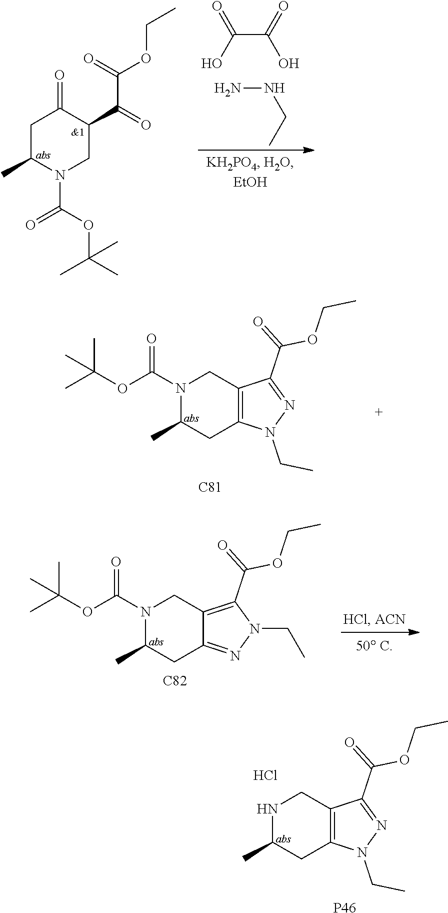

### Step 1. Preparation of 5-(tert-butyl) 3-ethyl (R)-1-ethyl-6-methyl-1,4,6,7-tetrahydro-5H-pyrazolo[4,3-c]pyridine-3,5-dicarboxylate (C81) and 5-(tert-butyl) 3-ethyl (R)-2-ethyl-6-methyl-2,4,6,7-tetrahydro-5H-pyrazolo[4,3-c]pyridine-3,5-dicarboxylate (C82)

A reaction mixture of H₂O (8.8 mL), EtOH (8.8 mL), tert-butyl (2S,5RS)-5-(2-ethoxy-2-oxoacetyl)-2-methyl-4-oxopiperidine-1-carboxylate (CAS: 2171296-27-6; 1.65 g, 5.27 mmol), ethylhydrazine oxalate (0.949 g, 6.32 mmol) and KH₂PO₄ (1.00 g, 7.37 mmol) was stirred at room temperature for 2 h. The yellow slurry was checked by LCMS, which showed C81 (LC/MS) m/z (M+H)⁺=338.3 at 1.69 min retention time and C82 (LC/MS) m/z (M+H)⁺=338.3 at 1.94 min retention time. The reaction mixture was diluted with H₂O and extracted with DCM (3×). The combined organic layer was dried with Na₂SO₄ and then purified by column chromatography (silica gel, 0-100% EtOAc:heptane) to provide C81 (1.53 g, 86.0% yield) and C82 (30.0 mg, 1.70% yield).

C81: ¹H NMR (400 MHz, (CD₃)₂SO) δ 4.88 (d, 1H), 4.78-4.65 (m, 1H), 4.33-4.17 (m, 2H), 4.14-3.94 (m, 3H), 2.85 (dd, 1H), 2.68-2.60 (m, 1H), 1.42 (s, 9H), 1.33-1.26 (m, 6H), 1.03 (d, 3H).

C82: ¹H NMR (400 MHz, (CD₃)₂SO) δ 4.91 (d, 1H), 4.78-4.63 (m, 1H), 4.54-4.36 (m, 2H), 4.35-4.15 (m, 2H), 4.09-3.96 (m, 2H), 3.38-3.26 (m, 1H), 2.82-2.65 (m, 2H), 2.56-2.51 (m, 1H), 2.47-2.36 (m, 1H), 2.28-2.12 (m, 2H), 1.35-1.27 (m, 6H), 1.10-1.06 (m, 3H), 1.04-0.99 (m, 3H).

### Step 2. Preparation of ethyl (R)-1-ethyl-6-methyl-4,5,6,7-tetrahydro-1H-pyrazolo[4,3-c]pyridine-3-carboxylate hydrochloride (P46)

To a solution of C81 (1.00 g, 2.96 mmol) in ACN (10 mL) was added 12.1M HCl (0.216 g, 5.93 mmol). The reaction mixture was stirred at 50° C. for 25 h and 15 min, then concentrated in vacuo. The residue was diluted with ACN and then concentrated in vacuo. The residue was diluted with DCM and concentrated in vacuo (2×) to provide P46 (0.730 g, crude) as a yellow foam. The yellow foam was used directly in the next step without further purification. (LC/MS) m/z (M+H)⁺=238.2. ¹H NMR (600 MHz, (CD₃)₂SO) δ 9.33-9.24 (m, 1H), 9.07-8.93 (m, 1H), 4.38-4.10 (m, 6H), 3.48-3.39 (m, 1H), 3.16 (dd, 1H), 2.71 (dd, 1H), 1.41-1.37 (m, 3H), 1.31 (dt, 6H).

### Preparation 47

### Methyl 3-methyl-5,6,7,8-tetrahydroimidazo[1,5-a]pyrazine-1-carboxylate (47)

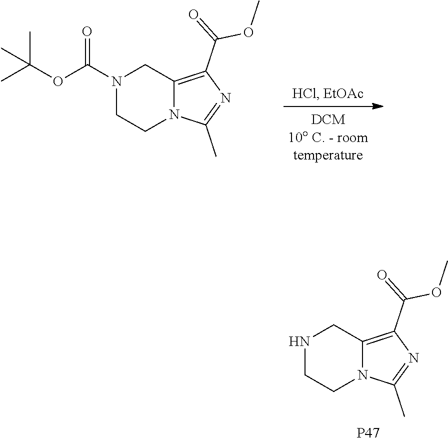 [SMILES: COC(=O)c1nc(C)n2c1CN(C(=O)OC(C)(C)C)CC2 ; "P47" ; COC(=O)c1nc(C)n2c1CNCC2]

### Step 1. Preparation of methyl 3-methyl-5,6,7,8-tetrahydroimidazo[1,5-a]pyrazine-1-carboxylate hydrochloride (P47)

To a solution of 7-(tert-butyl) 1-methyl 3-methyl-5,6-dihydroimidazo[1,5-a]pyrazine-1,7(8H)-dicarboxylate (CAS: 1359655-89-2, 0.190 g, 0.643 mmol) in DCM (5.0 mL) was added 2M HCl in EtOAc (5.0 mL) at 10° C. The reaction mixture was stirred at room temperature for 16 h. The reaction mixture became a white suspension concentrated in vacuo to provide the P47 (0.149 g, crude) as a white solid. This was used in the next step without further purification. ¹H NMR (400 MHz, CD₃OD) δ 4.80 (s, 2H), 4.51 (t, 2H), 4.00 (s, 3H), 3.84 (t, 2H), 2.71 (s, 3H).

### Preparation 48

### Ethyl 3-isopropyl-5,6,7,8-tetrahydroimidazo[1,5-a]pyrazine-1-carboxylate hydrochloride (P48)

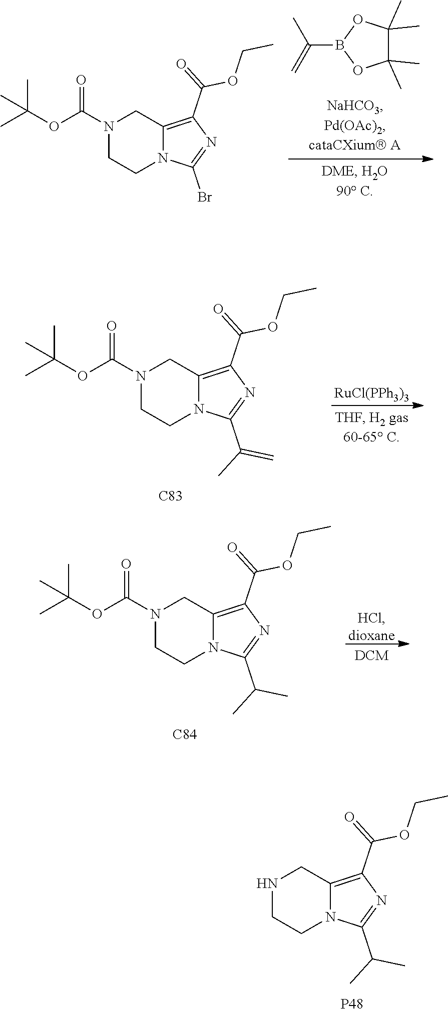

### Step 1. Preparation of 7-(tert-butyl) 1-ethyl 3-(prop-1-en-2-yl)-5,6-dihydroimidazo[1,5-a]pyrazine-1,7(8H)-dicarboxylate (C83)

A solution of 7-(tert-butyl) 1-ethyl 3-bromo-5,6-dihydroimidazo[1,5-a]pyrazine-1,7(8H)-dicarboxylate (CAS: 2108354-93-2; 1.00 g, 2.67 mmol), 4,4,5,5-tetramethyl-2-(prop-1-en-2-yl)-1,3,2-dioxaborolane (CAS: 126726-62-3, 0.808 g, 4.81 mmol), NaHCO₃ (0.673 g, 8.02 mmol), cataCXium® A (0.192 g, 0.534 mmol) and Pd(OAc)₂ (60.0 mg, 0.267 mmol) in (4:1) DME:H₂O (16 mL: 4 mL) was degassed with nitrogen gas for 5 min. The reaction solution was stirred at 90° C. for 16 h and then concentrated in vacuo. The residue was dissolved in DCM (50 mL) and then extracted with EtOAc (3×50 mL). The combined organic layer was dried with Na₂SO₄, filtered, and then concentrated in vacuo to give a residue. The residue was purified by column chromatography (silica gel, 0-20% EtOAc:PE) to provide C83 (0.856 g, 95.5% yield) as a light-yellow solid. (LC/MS) m/z (M+H)⁺=336.2.

### Step 2. Preparation of 7-(tert-butyl) 1-ethyl 3-isopropyl-5,6-dihydroimidazo[1,5-a]pyrazine-1,7(8H)-dicarboxylate (C84)

To the suspension of C83 (0.86 g, 2.5 mmol) in THF (20 mL) was added RuCl(PPh₃)₃ (0.24 g, 0.25 mmol). The reaction mixture was degassed with hydrogen gas and then stirred under a hydrogen atmosphere (50 psi) from 60-65° C. for 48 h. The suspension was concentrated in vacuo, and then the residue was purified by column chromatography (silica gel, 0-20% THF:PE) to provide C84 (0.67 g, 77% yield) as a brown solid. (LC/MS) m/z (M+H)⁺=338.3. ¹H NMR (400 MHz, CDCl₃) δ 4.87 (s, 2H), 4.36 (q, 2H), 3.92 (t, 2H), 3.82 (t, 2H), 3.03-2.89 (m, 1H), 1.50 (s, 9H), 1.43-1.32 (m, 9H).

### Step 3. Preparation of ethyl 3-isopropyl-5,6,7,8-tetrahydroimidazo[1,5-a]pyrazine-1-carboxylate hydrochloride (P48)

To a solution of C84 (0.67 g, 2.0 mmol) in DCM (10 mL) was added 2M HCl in dioxane (20 mL). The reaction solution was stirred at room temperature for 2 h then was concentrated in vacuo and was dried further by high vacuo to provide P48 (0.54 g, crude) as a solid. The solid was used directly in the next step without further purification.

### Preparation 49

### 5,7-Di-tert-butyl-2-(oxetan-3-yloxy)-3-phenyl-2,3-dihydrobenzo[d]oxazole (P49)

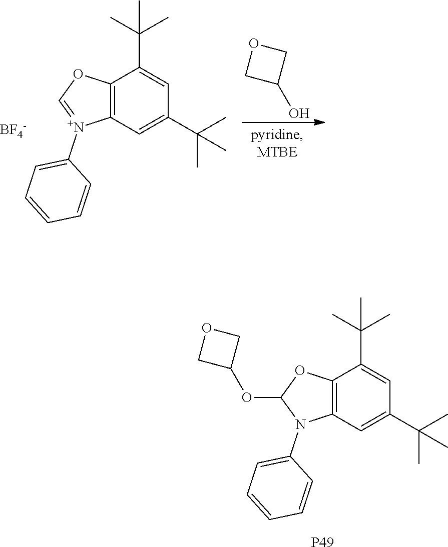 [SMILES: "BF4-" ; CC(C)(C)c1cc(C(C)(C)C)c2oc[n+](-c3ccccc3)c2c1 ; OC1COC1 ; "P49" ; CC(C)(C)c1cc2c(c(C(C)(C)C)c1)OC(OC1COC1)N2c1ccccc1]

### Step 1. Preparation of 5,7-di-tert-butyl-2-(oxetan-3-yloxy)-3-phenyl-2,3-dihydrobenzo[d]oxazole (P49)

A reaction mixture of 3-hydroxyoxetane (CAS: 7748-36-9; 41 mg, 0.55 mmol) and deoxazole (CAS: 1207294-92-5; 0.22 g, 0.55 mmol) in MTBE (3 mL) was degassed with nitrogen gas (2×). The suspension was stirred at room temperature for 5 min before adding pyridine (43 mg, 0.55 mmol). The reaction mixture was stirred for 30 min to provide a solution of P49 (0.21 g, crude). The solution was used directly in the next step without further purification.

### Preparation 50

### rac-Methyl (4R)-1-isopropyl-4-methyl-3a,4,5,6,7,7a-hexahydro-1H-pyrazolo[4,3-c]pyridine-3-carboxylate (P50)

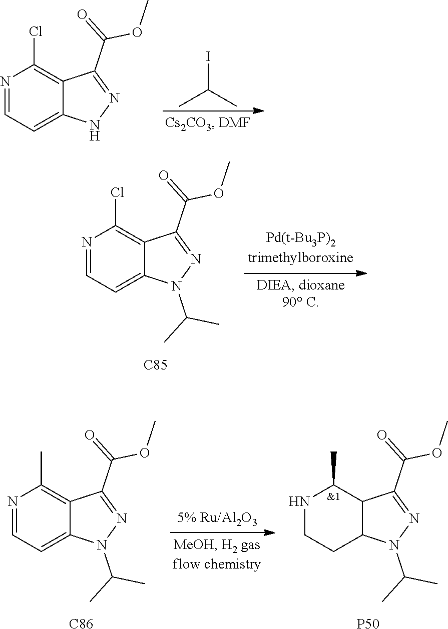

### Step 1. Preparation of methyl 4-chloro-1-isopropyl-1H-pyrazolo[4,3-c]pyridine-3-carboxylate P (C85)

To a solution of methyl 4-chloro-1H-pyrazolo[4,3-c]pyridine-3-carboxylate (CAS: 1658466-48-8; 6.00 g, 28.3 mmol) in DMF (120 mL) was added Cs₂CO₃ (18.5 g, 56.7 mmol) and 2-iodopropane (CAS: 75-30-9; 7.23 g, 42.5 mmol). The reaction mixture was stirred at room temperature for 3 h, diluted with EtOAc (100 mL), and washed with brine (3×300 mL). The organic layer was dried with Na₂SO₄ and then concentrated in vacuo. The yellow oil was purified by column chromatography (silica gel, 0-37% EtOAc:hexane) to provide C85 (3.30 g, 45.9% yield). (LC/MS) m/z (M+H)⁺=254.0. ¹H NMR (400 MHz, (CD₃)₂SO) δ 8.26 (d, 1H), 7.93 (d, 1H), 5.22-5.07 (m, 1H), 3.93 (s, 3H), 1.49 (d, 6H).

### Step 2. Preparation of methyl 1-isopropyl-4-methyl-1H-pyrazolo[4,3-c]pyridine-3-carboxylate (C86)

To a solution of C85 (2.10 g, 8.28 mmol), trimethylboroxine (CAS: 823-96-1; 8.31 g, 33.1 mmol) in dioxane (60 mL) was added DIEA (4.28 g, 33.1 mmol) and Pd(t-Bu₃P)₂ (0.635 g, 1.24 mmol). The reaction mixture was degassed with nitrogen gas and then stirred at 90° C. for 12 h. The suspension was diluted with EtOAc (100 mL), washed with brine (2×150 mL), dried with Na₂SO₄, filtered, and concentrated in vacuo. The residue was purified by column chromatography (silica gel, 0-50% EtOAc:hexane) to provide C86 (2.10 g, crude) as an orange gum. The gum was used directly in the next step without further purification. (LC/MS) m/z (M+H)⁺=234.0. ¹H NMR (400 MHz, CDCl₃) δ 8.33 (d, 1H), 7.24 (d, 1H), 4.96-4.83 (m, 1H), 4.03 (s, 3H), 3.06 (s, 3H), 1.65-1.61 (m, 6H).

### Step 3. Preparation of rac-methyl (4R)-1-isopropyl-4-methyl-3a,4,5,6,7,7a-hexahydro-1H-pyrazolo[4,3-c]pyridine-3-carboxylate (P50)

The reaction was carried out via flow chemistry. C86 (1.80 g, 7.72 mmol) was dissolved in MeOH (40 mL). The solution was pumped with a flow rate of 0.3 mL/min, and the H₂ had a 30 mL/min rate. The solution passed through the fixed bed (¼″ mm) packed with granular catalyst 5% Ru/Al₂O₃ with a 5.0 mL volume and was hydrogenated at 60° C. via flow at 2.0 MPa H₂. The reaction mixture was collected from the reactor output to provide P50 (1.85 g, crude) as a black oil. The oil was used directly in the next step without further purification. (LC/MS) m/z (M+H)⁺=238.2. ¹H NMR (400 MHz, (CD₃)₂SO) δ 4.61-4.42 (m, 2H), 4.24-4.01 (m, 1H), 3.86-3.75 (m, 3H), 3.04-2.77 (m, 4H), 1.49 (d, 3H), 1.38 (dd, 6H).

### Preparation 51

### rac-Methyl (4R)-4-ethyl-1-isopropyl-3a,4,5,6,7,7a-hexahydro-1H-pyrazolo[4,3-c]pyridine-3-carboxylate (P51)

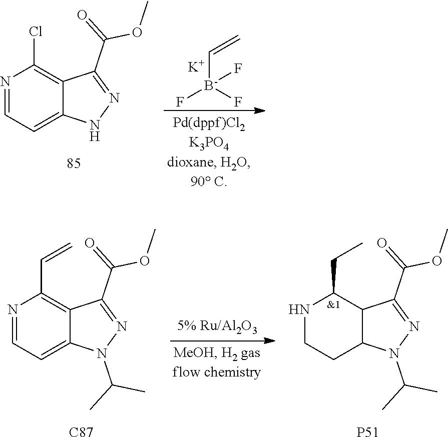

### Step 1. Preparation of methyl 1-isopropyl-4-vinyl-1H-pyrazolo[4,3-c]pyridine-3-carboxylate (C87)

To a reaction mixture of C85 (1.00 g, 3.94 mmol), potassium vinyltrifluoroborate (CAS: 13682-77-4; 0.792 g, 5.91 mmol) and K₃PO₄ (2.09 g, 9.85 mmol) in dioxane (10.0 mL) and H₂O (3.5 mL) was added Pd(dppf)Cl₂ (0.288 g, 0.394 mmol). The suspension was degassed with nitrogen gas for 3 min and then stirred at 90° C. for 2 h. The reaction mixture was filtered, and then the filter cake was washed with EtOAc (50 mL). The filtrate was concentrated in vacuo. The yellow gum was purified by column chromatography (silica gel, 0-40% EtOAc:hexane) to provide C87 (0.700 g, 72.4% yield) as a light yellow solid. (LC/MS) m/z (M+H)⁺=246.1. ¹H NMR (400 MHz, (CD₃)₂SO) δ 8.45 (d, 1H), 8.07 (dd, 1H), 7.79 (d, 1H), 6.51 (dd, 1H), 5.59 (dd, 1H), 5.19-5.08 (m, 1H), 3.93 (s, 3H), 1.50 (d, 6H).

### Step 2. Preparation of rac-methyl (4R)-4-ethyl-1-isopropyl-3a,4,5,6,7,7a-hexahydro-1H-pyrazolo[4,3-c]pyridine-3-carboxylate (P51)

The reaction was carried out via flow chemistry. C87 (0.300 g, 1.22 mmol) was dissolved in MeOH (20 mL). The solution was pumped with a flow rate of 0.3 mL/min, and the H₂ had a 30 mL/min rate. The solution passed through the fixed bed (¼″ mm) packed with granular catalyst 5% Ru/Al₂O₃ with a 5.0 mL volume and was hydrogenated at 60° C. via flow at 2.0 MPa H₂. The reaction mixture was collected from the reactor output to provide P51 (0.280 g, crude) as a brown oil. The oil was used directly in the next step without further purification. (LC/MS) m/z (M+H)⁺=252.0. ¹H NMR (400 MHz, (CD₃)₂SO) δ 9.75 (s, 1H), 9.23 (s, 1H), 4.59-4.50 (m, 1H), 3.78 (s, 3H), 3.07-2.89 (m, 2H), 2.45-2.41 (m, 2H), 2.00-1.75 (m, 2H), 1.35 (dd, 6H), 1.02 (t, 3H).

### Preparation 52

### rac-Ethyl (6R,7R)-1-isopropyl-6,7-dimethyl-4,5,6,7-tetrahydro-1H-pyrazolo[4,3-c]pyridine-3-carboxylate (P52)

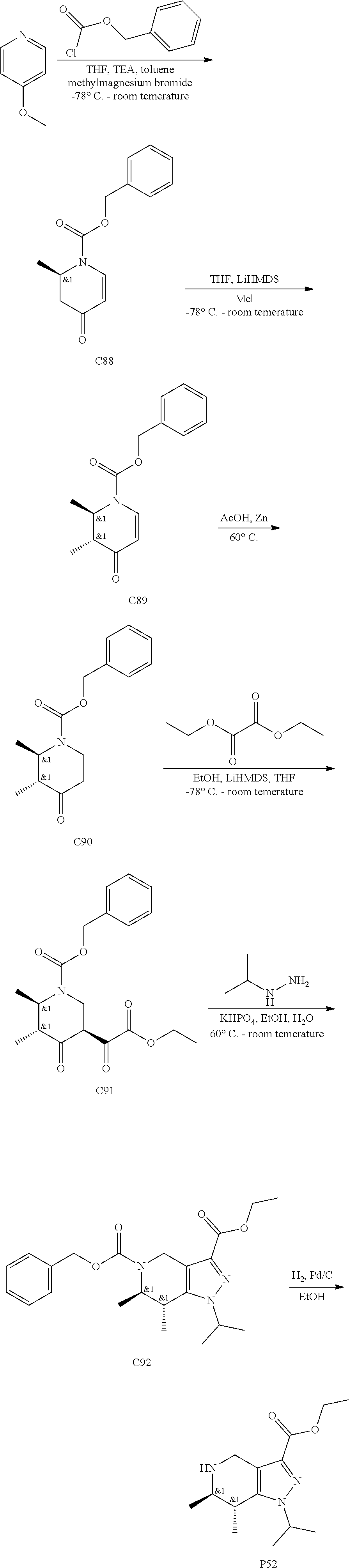

### Step 1. Preparation of rac-benzyl (R)-2-methyl-4-oxo-3,4-dihydropyridine-1(2H)-carboxylate (C88)

To a round bottom flask was added 4-methoxypyridine (CAS: 620-08-6; 32.6 mL), THF (641.4 mL), and TEA (4.5 mL) sequentially. The solution was cooled to −78° C., and then 3M benzyl chloroformate in toluene (109.0 mL) was added dropwise to give a white slurry. 3.4M methylmagnesium bromide solution (55.6 mL) dropwise was added to the suspension. The reaction mixture was stirred at −78° C. and slowly warmed to room temperature over 24 h. The reaction mixture was cooled to 0° C. and quenched via a portion-wise addition of 1M aqueous HCl. The aqueous mixture was extracted with DCM (3×). The organic layer was combined, then dried with Na₂SO₄ and purified by column chromatography (silica gel, 0-50% EtOAc:heptane) to provide C88 (51.5 g, 65.5% yield) as a clear oil. (LC/MS) m/z (M+H)⁺=246.4. ¹H NMR (400 MHz, (CD₃)₂SO) δ 7.78 (dd, 1H), 7.48-7.28 (m, 5H), 5.33-5.21 (m, 3H), 4.68-4.56 (m, 1H), 2.89 (dd, 1H), 2.24-2.14 (m, 1H), 1.15 (d, 3H).

### Step 2. Preparation of rac-benzyl (2R,3R)-2,3-dimethyl-4-oxo-3,4-dihydropyridine-1(2H)-carboxylate (C89)

A reaction mixture of C88 (10.0 g, 40.8 mmol) in THF (102 mL) was cooled to −78° C., then 1M LiHMDS in THF (42.8 mL) was added dropwise and stirred at −78° C. for 10 min. After stirring for a few min, Mel (3.1 mL) was added dropwise at −78° C. to the reaction mixture and then warmed to room temperature over 30 min. The suspension was stirred at room temperature for 9 h and then quenched with brine. The pH of the aqueous layer was adjusted to pH=1 using 12M aqueous HCl. The reaction mixture was extracted with EtOAc (2×), and the organic layers were combined and then dried with Na₂SO₄. The mixture was purified by column chromatography (silica gel, 0-40% MTBE:heptane) to provide C89 (7.80 g, 73.6% yield) as a yellow oil. ¹H NMR (400 MHz, CD₃OD) δ 7.84 (dd, 1H), 7.47-7.32 (m, 5H), 5.36-5.20 (m, 3H), 4.40 (q, 1H), 2.27 (q, 1H), 1.21 (d, 3H), 1.15 (d, 3H).

### Step 3. Preparation of rac-benzyl (2R,3R)-2,3-dimethyl-4-oxopiperidine-1-carboxylate (C90)

To a round bottom flask was added C89 (12.8 g, 49.4 mmol), AcOH (82.3 mL), and Zn (12.9 g, 197 mmol) sequentially, then stirred at 60° C. for 5 h. The reaction mixture was concentrated in vacuo, then saturated NaHCO₃ and extracted with DCM (3×). The combined organic layer was purified by column chromatography (silica gel, 0-50% EtOAc:heptane) to provide C90 (10.1 g, 78.3% yield) as a clear oil. ¹H NMR (400 MHz, (CD₃)₂SO) δ 7.44-7.28 (m, 5H), 5.17-5.06 (m, 2H), 4.13 (dd, 1H), 4.07-3.96 (m, 1H), 3.53-3.41 (m, 1H), 2.57-2.44 (m, 2H), 2.33-2.23 (m, 1H), 1.21 (d, 3H), 1.05 (d, 3H).

### Step 4. Preparation of rac-benzyl (2R,3R,5R)-5-(2-ethoxy-2-oxoacetyl)-2,3-dimethyl-4-oxopiperidine-1-carboxylate (C91)

A reaction mixture of C90 (0.430 g, 1.65 mmol), EtOH (3.29 mL), and diethyl oxalate (0.24 mL) was cooled to −78° C., then 1M LiHMDS in THF (1.89 mL) was added. The suspension was stirred at room temperature for 17 h and then quenched with a mixture of 1M aqueous HCl, brine, and DCM. The aqueous layer was extracted with (2:1, MTBE:DCM, 2×). The organic layers were combined and then dried with Na₂SO₄ and concentrated in vacuo to provide C91 (0.590 g, crude) as a yellow oil. The oil was used directly in the next step without further purification.

### Step 5. Preparation of rac-5-benzyl 3-ethyl (6R,7R)-1-isopropyl-6,7-dimethyl-1,4,6,7-tetrahydro-5H-pyrazolo[4,3-c]pyridine-3,5-dicarboxylate (C92)

A reaction mixture of C91 (0.590 g, 1.63 mmol), KHPO₄ (0.311 g, 2.29 mmol) and isopropylhydrazine HCl (217 mg, 1.96 mmol) in EtOH (2.7 mL) and H₂O (2.7 mL) was stirred at 60° C. for 2.5 h then stirred at room temperature for 24 h. The suspension was diluted with H₂O and then extracted with DCM (3×). The organic layers were combined and then purified with column chromatography (silica gel, 0-60% EtOAc:heptane) to provide C92 (0.322 g, 49.4% yield) as a clear oil.

### Step 6. Preparation of rac-ethyl (6R,7R)-1-isopropyl-6,7-dimethyl-4,5,6,7-tetrahydro-1H-pyrazolo[4,3-c]pyridine-3-carboxylate (P52)

In a reactor was added Pd/C (10 wt %, 0.213 g, 2.00 mmol) in EtOH (1 mL), then added solution of C92 (0.800 g, 2.00 mmol) in EtOH (5 mL). The suspension was degassed with hydrogen gas and then stirred under a hydrogen atmosphere (120 psi) at room temperature for 1 h. The reaction mixture was filtered through Celite and then washed with tert-amyl alcohol. The filtrate was concentrated in vacuo to provide P52 (0.531 g, crude) as a yellow oil. The oil was used directly in the next step without further purification. ¹H NMR (400 MHz, (CD₃)₂SO) δ 4.57-4.46 (m, 1H), 4.23 (q, 2H), 3.82-3.68 (m, 2H), 2.84-2.74 (m, 1H), 2.62-2.55 (m, 1H), 1.43 (d, 3H), 1.39-1.31 (m, 6H), 1.22-1.18 (m, 3H), 1.05-1.01 (m, 3H).

### Preparation 53

### rac-Ethyl (R)-3-isopropyl-5-methyl-5,6,7,8-tetrahydroimidazo[1,5-a]pyrazine-1-carboxylate hydrochloride (P53)

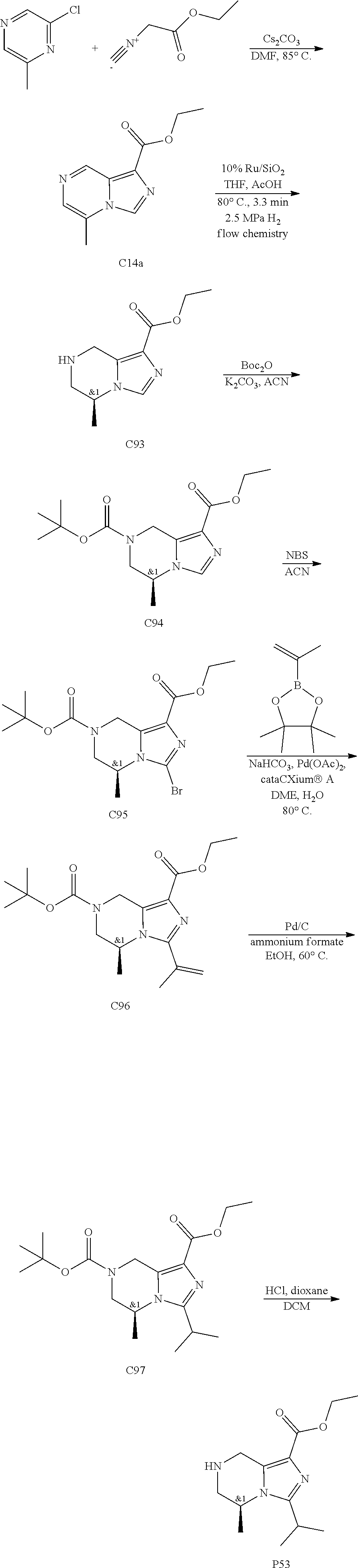

### Step 1. Preparation of ethyl 5-methylimidazo[1,5-a]pyrazine-1-carboxylate (C14a)

To a mixture of 2-chloro-6-methylpyrazine (CAS: 38557-71-0, 10.0 g, 77.8 mmol) in DMF (150.0 mL) was added Cs₂CO₃ (38.0 g, 117 mmol) and ethyl 2-isocyanoacetate (CAS: 2999-46-4, 17.8 g, 156 mmol) at 15° C. The reaction was heated to 85° C. for 16 h, then diluted with EtOAc (300 mL). The light brown reaction mixture was filtered, and the filtrate was concentrated in vacuo. The brown residue was diluted with (1:1, EtOAc:THF) and filtered. The filter cake was dissolved in (1:1, H₂O:EtOAc) and then extracted with EtOAc (150 mL). The combined organic phase was dried with Na₂SO₄ and concentrated in vacuo. The residue was purified by column chromatography (silica gel, 0-100% EtOAC:PE) to provide C14a (7.71 g, 48.3% yield) as a light-brown solid. (LC/MS) m/z (M+H)⁺=206.1. ¹H NMR (400 MHz, CDCl₃) δ 9.54 (s, 1H), 8.16 (s, 1H), 7.66 (s, 1H), 4.52 (q, 2H), 2.65 (s, 3H), 1.48 (t, 3H).

### Step 2. Preparation of rac-ethyl (R)-5-methyl-5,6,7,8-tetrahydroimidazo[1,5-a]pyrazine-1-carboxylate acetate (C93)

The reaction was carried out via flow chemistry. C14a (7.71 g, 37.6 mmol) was dissolved in THF (155 mL) and AcOH (40 mL) and then was pumped with a flow rate of 0.3 mL/min, and the H₂ had a rate of 30 mL/min. The solution passed through the fixed bed ((¼″) mm) packed with granular catalyst 10% Ru/SiO₂ (2.80 g, 2.76 mmol) with a 5.0 mL volume and was hydrogenated for 3.3 min at 80° C. via flow at 2.5 MPa H₂. The flow rate of H₂ was 100 mL/min. The reaction mixture was collected after 10 min and then concentrated in vacuo to remove THF. The suspension was stirred for 1-2 h and then filtered. The filtrate was concentrated in vacuo to provide C93 (5.50 g, crude) as a light-brown oil. The oil was used directly in the next step without further purification. (LC/MS) m/z (M+H)⁺=210.1.

### Step 3. Preparation of rac-7-(tert-butyl) 1-ethyl (R)-5-methyl-5,6-dihydroimidazo[1,5-a]pyrazine-1,7(8H)-dicarboxylate (C94)

To a solution of C93 (7.50 g, 19.3 mmol) in ACN (100 mL) was added Boc₂O (6.31 g, 28.9 mmol) and K₂CO₃ (10.7 g, 77.1 mmol) at room temperature. The reaction was stirred at room temperature for 16 h and then diluted with EtOAc (100 mL). The suspension was filtered then the filtrate was concentrated in vacuo. The residue was purified by column chromatography (silica gel; 0-100% EtOAc:PE) to provide C94 (5.30 g, 93.1% yield) as an off-white solid. (LC/MS) m/z (M+H)⁺=310.2. ¹H NMR (400 MHz, CDCl₃) δ 7.53 (s, 1H), 4.96-4.78 (m, 2H), 4.41-4.22 (m, 3H), 3.95-3.90 (m, 1H), 3.48-3.43 (m, 1H), 1.55-1.49 (m, 12H), 1.39 (t, 3H).

### Step 4. Preparation of rac-7-(tert-butyl) 1-ethyl (R)-3-bromo-5-methyl-5,6-dihydroimidazo[1,5-a]pyrazine-1,7(8H)-dicarboxylate (C95)

To a solution of C94 (3.20 g, 10.8 mmol) in ACN (30.0 mL) was added a solution of NBS (2.89 g, 16.3 mmol) in ACN (20.0 mL). The reaction mixture was stirred at room temperature for 16 h and then concentrated in vacuo. The residue was purified by column chromatography (silica gel, 0-40% EtOAc:PE) to provide C95 (2.57 g, 64.0% yield) as a white solid. (LC/MS) m/z (M+2H)⁺=390.0. ¹H NMR (400 MHz, CDCl₃) δ 5.59-5.25 (m, 1H), 4.51-4.25 (m, 5H), 3.40-3.12 (m, 1H), 1.51 (s, 9H), 1.45-1.29 (m, 6H).

### Step 5. Preparation of rac-7-(tert-butyl) 1-ethyl (R)-3-isopropyl-5-methyl-5,6-dihydroimidazo[1,5-a]pyrazine-1,7(8H)-dicarboxylate (C96)

A solution of C95 (1.00 g, 2.58 mmol), 4,4,5,5-tetramethyl-2-(prop-1-en-2-yl)-1,3,2-dioxaborolane (CAS: 126726-62-3, 0.779 g, 4.64 mmol), NaHCO₃ (0.649 g, 7.73 mmol), cataCXium® A (0.185 g, 0.515 mmol) and Pd(OAc)₂ (57.8 mg, 0.258 mmol) in (4:1) DME:H₂O (16 mL: 4 mL) was degassed with nitrogen gas for 5 min. The reaction solution was stirred at 80° C. for 15 h, then filtered and concentrated in vacuo. The residue was purified by column chromatography (silica gel, 0-35% EtOAc:PE) to provide C96 (0.810 g, 90.0% yield) as a gum. (LC/MS) m/z (M+H)⁺=350.2. ¹H NMR (400 MHz, (CD₃)₂SO) δ 5.49-5.38 (m, 2H), 5.26-5.05 (m, 1H), 4.79-4.65 (m, 1H), 4.58-4.36 (m, 1H), 4.33-3.97 (m, 4H), 2.10-2.06 (m, 3H), 1.43 (s, 9H), 1.31-1.24 (m, 3H), 1.23-1.16 (m, 3H).

### Step 6. Preparation of rac-7-(tert-butyl) 1-ethyl (R)-5-methyl-3-(prop-1-en-2-yl)-5,6-dihydroimidazo[1,5-a]pyrazine-1,7(8H)-dicarboxylate (C97)

To a solution of C96 (0.810 g, 2.32 mmol) in EtOH were added ammonium formate (2.19 g, 34.8 mmol) and Pd/C (0.370 g, 0.348 mmol). The reaction mixture was stirred at 60° C. under argon gas for 1 h and then filtered through celite. The filter cake was washed with EtOH (3×30 mL). The filtrate was concentrated in vacuo and then dissolved in DCM (50 mL). The solution was washed with brine (3×50 mL). The organic layer was concentrated in vacuo to provide C97 (0.715 g, crude). The crude was used directly into the next step without further purification. (LC/MS) m/z (M+H)⁺=352.2.

### Step 7. Preparation of rac-ethyl (R)-3-isopropyl-5-methyl-5,6,7,8-tetrahydroimidazo[1,5-a]pyrazine-1-carboxylate hydrochloride (P53)

To a solution of C97 (0.715 g, 2.03 mmol) in DCM (10 mL) was added 2M HCl in dioxane (20 mL). The reaction mixture was stirred for 2 h at room temperature, then concentrated in vacuo and lyophilized to provide P53 (0.710 g, crude) as a gum. The gum was used directly in the next step without further purification. (LC/MS) m/z (M+H)⁺=252.2.

### Example 1

### N-(4-(3,3-dimethylureido)benzyl)-1-isopropyl-5-(5-(2-oxopyrrolidin-1-yl)pyridin-2-yl)-4,5,6,7-tetrahydro-1H-pyrazolo[4,3-c]pyridine-3-carboxamide (1)

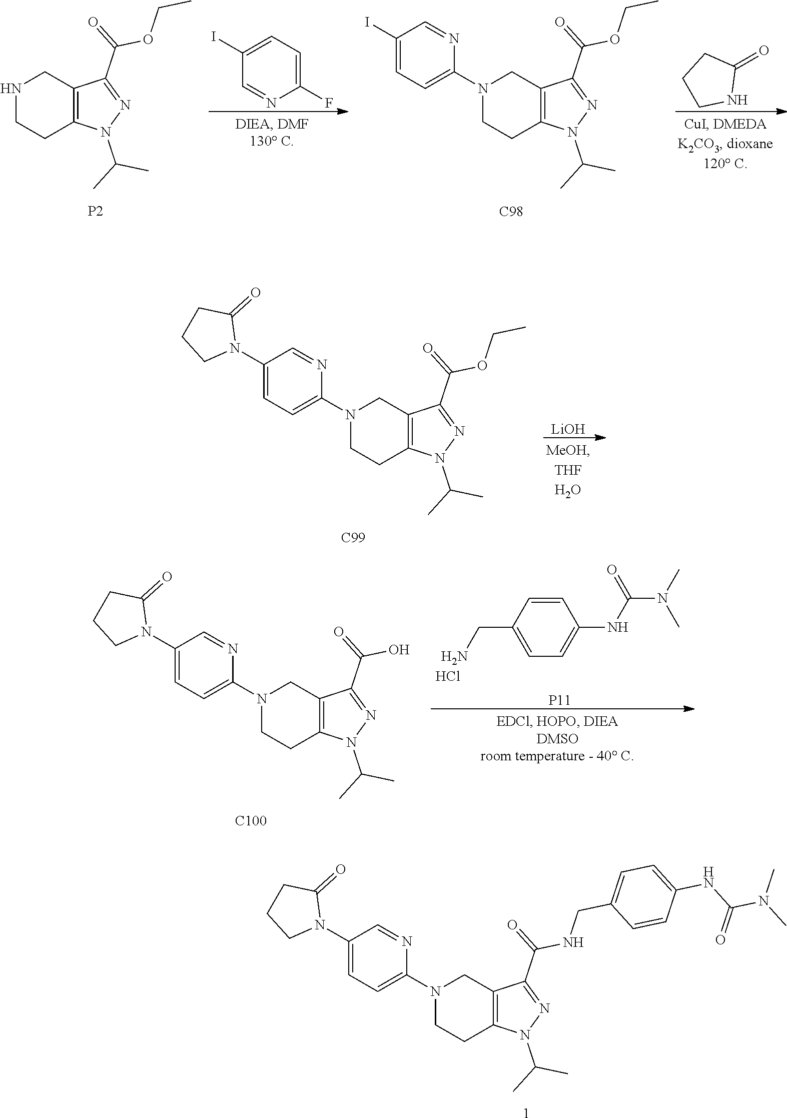

### Step 1. Preparation of ethyl 5-(5-iodopyridin-2-yl)-1-isopropyl-4,5,6,7-tetrahydro-1H-pyrazolo[4,3-c]pyridine-3-carboxylate (C98)

A mixture of P2 (1.36 g, 4.98 mmol), 2-fluoro-5-iodopyridine (CAS: 171197-80-1, 1.22 g, 5.48 mmol), and DIEA (1.93 g, 14.9 mmol) in DMF (25.0 mL) was stirred at 130° C. for 16 h. The brown reaction mixture was concentrated in vacuo and purified by column chromatography (silica gel, 0-20% EtOAc:PE) to provide C98 (0.61 g, 27.8%) as a white solid. (LC/MS) m/z (M+H)⁺=441.2. ¹H NMR (400 MHz, CDCl₃) δ 8.32-8.31 (m, 1H), 7.68 (dd, 1H), 6.58 (d, 1H), 4.60 (s, 2H), 4.52-4.38 (m, 3H), 4.03 (t, 2H), 2.80 (t, 2H), 1.52 (d, 6H), 1.44-1.40 (m, 3H).

### Step 2. Preparation of ethyl 1-isopropyl-5-(5-(2-oxopyrrolidin-1-yl)pyridin-2-yl)-4,5,6,7-tetrahydro-1H-pyrazolo[4,3-c]pyridine-3-carboxylate (C99)

A mixture of C98 (0.400 g, 0.909 mmol), pyrrolidine-2-one (0.116 g, 1.36 mmol), CuI (34.6 mg, 0.182 mmol), DMEDA (16.0 mg, 0.182 mmol) and K₂CO₃ (0.377 g, 2.73 mmol) in dioxane (10.0 mL) was heated at 120° C. for 16 h under nitrogen. The reaction was concentrated in vacuo, and then the residue was purified by column chromatography (silica gel, 0-30% THF:PE) to provide C99 (0.36 g, 99.7% yield) as a white solid. (LC/MS) m/z (M+H)⁺=398.2.

### Step 3. Preparation of 1-isopropyl-5-(5-(2-oxopyrrolidin-1-yl)pyridin-2-yl)-4,5,6,7-tetrahydro-1H-pyrazolo[4,3-c]pyridine-3-carboxylic acid (C100)

To a solution of C99 (0.360 g, 0.906 mmol) in THF (5.00 mL), MeOH (1.25 mL) and H₂O (2.50 mL) was added LiOH (57.5 mg, 2.40 mmol) then stirred at room temperature for 1 h and 30 min. The solution was concentrated in vacuo and then acidified to pH=5-6 with 2N HCl. An off-white solid precipitated was filtered, then washed with H₂O (3×50 mL), dried, then lyophilized to provide C100 (0.27 g, 80.7%) as a white solid. ¹H NMR (400 MHz, CD₃OD) δ 8.29 (d, 1H), 7.82 (dd, 1H), 6.91 (d, 1H), 4.67 (s, 2H), 4.60-6.45 (m, 1H), 3.96 (t, 2H), 3.88-3.83 (m, 2H), 2.86 (t, 2H), 2.60-2.52 (m, 2H), 2.24-2.13 (m, 2H), 1.47 (d, 6H).

### Step 4. Preparation of N-(4-(3,3-dimethylureido)benzyl)-1-isopropyl-5-(5-(2-oxopyrrolidin-1-yl)pyridin-2-yl)-4,5,6,7-tetrahydro-1H-pyrazolo[4,3-c]pyridine-3-carboxamide (1)

A solution of C100 (80.0 mg, 0.217 mmol), HOPO (71.0 mg, 0.639 mmol), EDCl (99.6 mg, 0.520 mmol) and DIEA (0.140 g, 1.08 mmol) in DMSO (2.0 mL) were stirred at room temperature for 10 min. to the reaction mixture was added P11 (74.6 mg, 0.325 mmol) then heated to 40° C. and stirred for 2 h. The residue was purified by reverse phase HPLC (C₁₈ 150 mm×30 mm×5 μm column, Mobile Phase A: H₂O/Mobile Phase B: ACN (NH₄OH—NH₄HCO₃), 18 to 58% of Mobile Phase B over 9 min, 100% of Mobile Phase B hold 2 min, flow rate: 30 mL/min) and lyophilized to provide 1 (72.2 mg, 61.2% yield) as a white solid. (LC/MS) m/z (M+H)⁺=545.4. ¹H NMR (400 MHz, (CD₃)₂SO) δ 8.33-8.27 (m, 2H), 8.23 (s, 1H), 7.89 (dd, 1H), 7.42-7.37 (m, 2H), 7.20-7.15 (m, 2H), 6.90 (d, 1H), 4.65 (s, 2H), 4.53-4.42 (m, 1H), 4.35 (d, 2H), 3.87 (t, 2H), 3.76 (t, 2H), 2.91 (s, 6H), 2.80 (t, 2H), 2.43 (t, 2H), 2.10-2.00 (m, 2H), 1.39 (d, 6H).

### Example 2

### 5-(5-(Dimethylcarbamoyl)pyridin-2-yl)-N-(4-(3,3-dimethylureido)benzyl)-1-isopropyl-4,5,6,7-tetrahydro-1H-pyrazolo[4,3-c]pyridine-3-carboxamide (2)

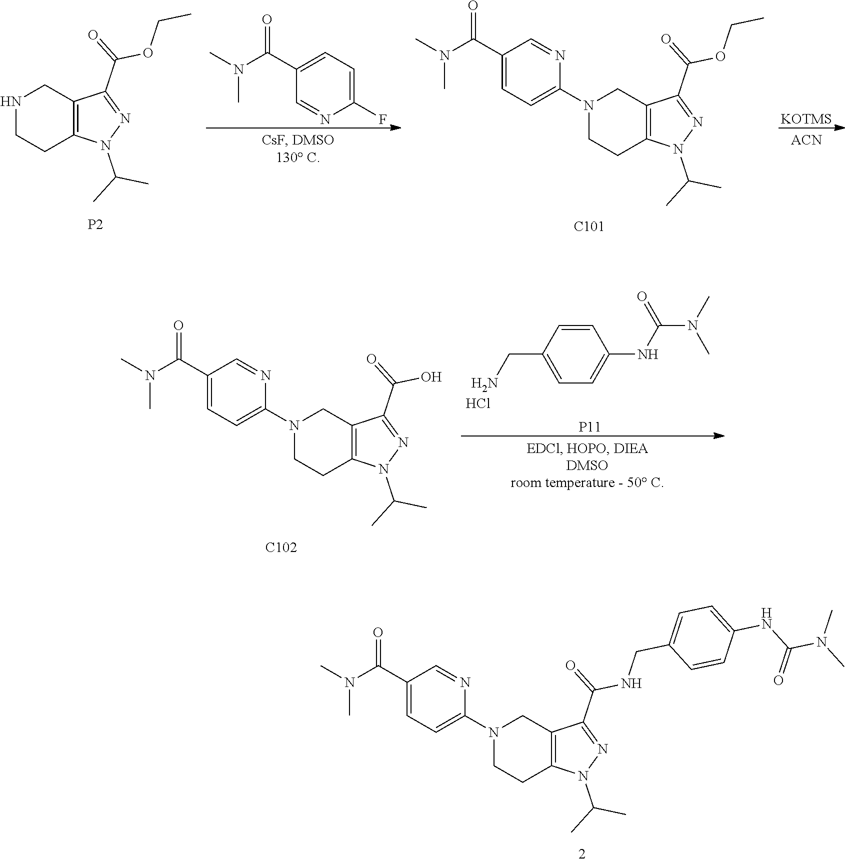

### Step 1. Preparation of ethyl 5-(5-(dimethylcarbamoyl)pyridin-2-yl)-1-isopropyl-4,5,6,7-tetrahydro-1H-pyrazolo[4,3-c]pyridine-3-carboxylate (C101)

To a mixture of P2 (70.0 g, 256 mmol) and CsF (97.1 g, 639 mmol) in DMSO (0.7 L) was added 6-fluoro-N,N-dimethylnicotinamide (CAS: 1032251-82-3, 57.3 g, 256 mmol). The reaction was stirred at 130° C. for 8 h. The reaction was diluted with H₂O (6 L) and then extracted with MeOH:DCM (1:10, 3×2 L). The combined organic layer was washed with brine (2×3 L), dried with Na₂SO₄, filtered, and concentrated in vacuo. The residue was purified by column chromatography (silica gel, 0-100% EtOAc:PE) to provide C101 (72.0 g, 73.1% yield) as a pale yellow solid. (LC/MS) m/z (M+H)⁺=386.2. ¹H NMR (600 MHz, (CD₃)₂SO) δ 8.25 (d, 1H), 7.65 (dd, 1H), 6.91 (d, 1H), 4.74 (s, 2H), 4.58-4.47 (m, 1H), 4.30 (q, 2H), 3.96 (t, 2H), 2.97 (s, 6H), 2.83 (t, 2H), 1.38 (d, 6H), 1.32 (t, 3H).

### Step 2. Preparation of 5-(5-(dimethylcarbamoyl)pyridin-2-yl)-1-isopropyl-4,5,6,7-tetrahydro-1H-pyrazolo[4,3-c]pyridine-3-carboxylic acid (C102)

The reaction was conducted in two batches then combined for purification. At room temperature under nitrogen, a mixture of C101 (42.0 g, 109 mmol) in ACN (420 mL) was added KOTMS (28.0 g, 218 mmol) then stirred at room temperature for 1 h to afford the first batch. The first batch was acidified with 2M HCl in dioxane (109 mL, 7.95 g, 218 mmol) to pH=5-6 then concentrated in vacuo.

The first batch's residue was combined with the second batch of the same reaction utilizing C101 (43.0 g, 112 mmol). The combined reaction mixture was diluted with brine (500 mL) and extracted with MeOH:DCM (1:10, 3×1 L). The combined organic layers were dried with Na₂SO₄, filtered, and then concentrated in vacuo to provide C102 (66.0 g, 83.7% yield) as a white solid. This was used in the next step without further purification. ¹H NMR (600 MHz, (CD₃)₂SO) δ 8.24 (d, 1H), 7.63 (dd, 1H), 6.92 (d, 1H), 4.70 (s, 2H), 4.54-4.37 (m, 1H), 3.94 (t, 2H), 2.97 (s, 6H), 2.79 (t, 2H), 1.37 (t, 6H).

### Step 3. Preparation of 5-(5-(dimethylcarbamoyl)pyridin-2-yl)-N-(4-(3,3-dimethylureido)benzyl)-1-isopropyl-4,5,6,7-tetrahydro-1H-pyrazolo[4,3-c]pyridine-3-carboxamide (2)

To C102 (70 mg, 0.20 mmol) dissolved in ACN (1.0 mL) and H₂O (0.1 mL) was added HOPO (26 mg, 0.24 mmol) and EDCl (60 mg, 0.31 mmol). The reaction was stirred for 20 min at 50° C. and then cooled to room temperature before DIEA (76 mg, 0.59 mmol) and P11 (54 mg, 0.24 mmol) were added to the reaction. The mixture was stirred at room temperature for 1 h and 20 min. The reaction was concentrated in vacuo, dissolved in DMSO, and acidified by TFA. The acidic residue was purified by reverse phase HPLC (Sunfire C₁₈ 100 mm×19 mm×5 μm column, Mobile Phase A: H₂O/Mobile Phase B: ACN (0.05% TFA), 15 to 95% of Mobile Phase B over 9.0 min, 95% of Mobile Phase B hold 1.0 min, flow rate: 25 mL/min) to provide 2 (30 mg, 25% yield). (LC/MS) m/z (M+H)⁺=533.4. ¹H NMR (600 MHz, (CD₃)₂SO) δ=8.27 (t, 1H), 8.18-8.12 (m, 2H), 7.58 (dd, 1H), 7.34-7.30 (m, 2H), 7.11 (d, 2H), 6.84 (d, 1H), 4.68 (s, 2H), 4.45-4.38 (m, 1H), 4.28 (d, 2H), 3.89 (t, 2H), 2.93-2.88 (m, 6H), 2.84 (s, 6H), 2.76 (t, 2H), 1.33 (d, 6H).

### Example 3

### 5-(5-(Dimethylcarbamoyl)pyridin-2-yl)-N-(4-(3,3-dimethylureido)benzyl)-1-ethyl-4,5,6,7-tetrahydro-1H-pyrazolo[4,3-c]pyridine-3-carboxamide (3)

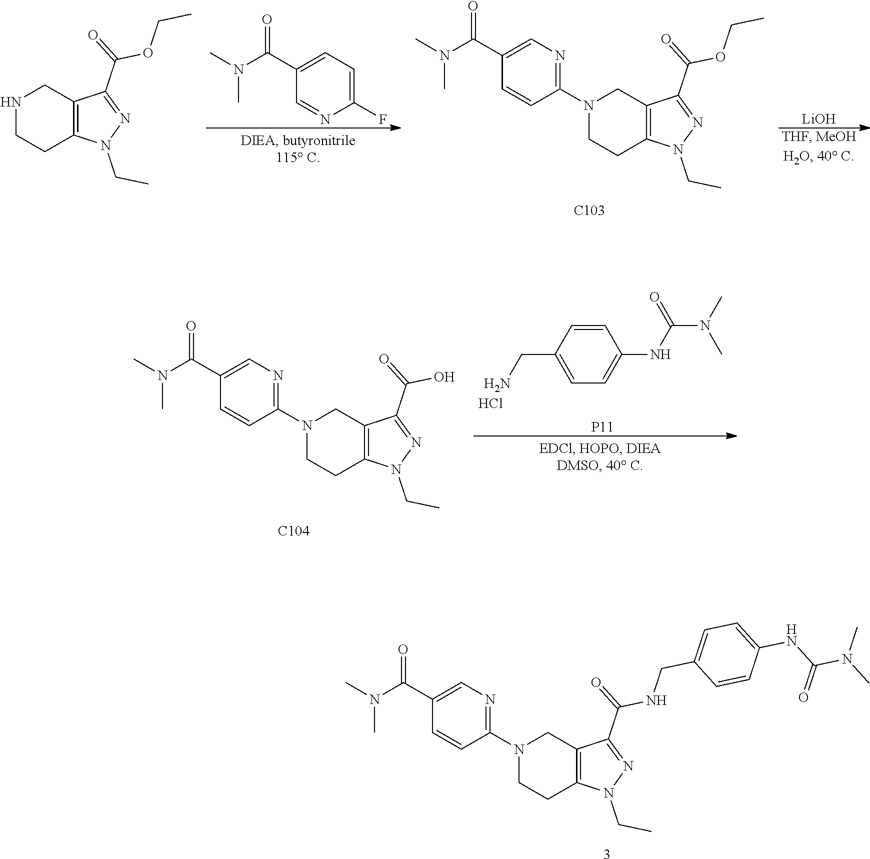

### Step 1. Preparation of ethyl 5-(5-(dimethylcarbamoyl)pyridin-2-yl)-1-ethyl-4,5,6,7-tetrahydro-1H-pyrazolo[4,3-c]pyridine-3-carboxylate (C103)

To a mixture of ethyl 1-ethyl-4,5,6,7-tetrahydro-1H-pyrazolo[4,3-c]pyridine-3-carboxylate hydrochloride (prepared using similar methods to Preparation 1, 0.350 g, 1.35 mmol) in butyronitrile (5.0 mL) and DIEA (0.697 g, 5.39 mmol) was added 6-fluoro-N,N-dimethylnicotinamide (CAS: 1032251-82-3, 0.249 g, 1.48 mmol) and reaction heated at 115° C. for 17 h. The solution was concentrated in vacuo, then redissolved in DCM, and purified by column chromatography (silica gel, 0-5% MeOH:DCM) to provide C103 (0.209 g, 41.7% yield) as a yellow oil. (LC/MS) m/z (M+H)⁺=372.4. ¹H NMR (600 MHz, (CD₃)₂SO) δ 8.23 (d, 1H), 7.64 (dd, 1H), 6.91 (d, 1H), 4.73 (s, 2H), 4.28 (q, 2H), 4.09 (q, 2H), 3.95 (t, 2H), 2.96 (s, 6H), 2.81 (t, 2H), 1.34-1.28 (m, 6H).

### Step 2. Preparation of 5-(5-(dimethylcarbamoyl)pyridin-2-yl)-1-ethyl-4,5,6,7-tetrahydro-1H-pyrazolo[4,3-c]pyridine-3-carboxylic acid (C104)

To C103 (0.250 g, 0.673 mmol) in THF (2.0 mL), H₂O (2.0 mL), MeOH (0.2 mL) was added LiOH (80.6 mg, 3.37 mmol) at 15° C. The reaction was heated to 40° C. and stirred for 1 h. The reaction was concentrated in vacuo to remove the organic layer, and the aqueous layer was acidified to pH=3 by 2N HCl. The acidic aqueous phase was concentrated in vacuo then purified by reverse phase HPLC (C₁₈ 150 mm×30 mm×5 μm column, Mobile Phase A: H₂O/Mobile Phase B: ACN (0.05% formic acid), 0 to 32% of Mobile Phase B over 9 min, 100% then Mobile Phase B hold 2 min, flow rate: 30 mL/min) and lyophilized to provide C104 (0.150 g, 64.9% yield) as a white solid. (LC/MS) m/z (M+H)⁺=344.3. ¹H NMR (400 MHz, CD₃OD) δ 8.31-8.25 (m, 1H), 7.68 (dd, 1H), 6.91 (d, 1H), 4.77 (s, 2H), 4.15 (q, 2H), 4.06 (t, 2H), 3.09 (s, 6H), 2.86 (t, 2H), 1.40 (t, 3H).

### Step 3. Preparation of 5-(5-(dimethylcarbamoyl)pyridin-2-yl)-N-(4-(3,3-dimethylureido)benzyl)-1-ethyl-4,5,6,7-tetrahydro-1H-pyrazolo[4,3-c]pyridine-3-carboxamide (3)

The reaction was conducted in two batches and then combined for purification. To a solution of C104 (0.100 g, 0.291 mmol) in DMSO (3.0 mL) was added HOPO (97.1 mg, 0.874 mmol), DIEA (0.188 g, 1.46 mmol), and EDCl (0.134 g, 0.699 mmol) to form the first batch. The reaction mixture of the first batch was stirred at 40° C. for 15 min before P11 (73.6 mg, 0.320 mmol) was added. The suspension of the first batch was stirred at 40° C. for 2 h.

The reaction mixture of the first batch was combined with a second batch of the same reaction utilizing C104 (0.563 g, 1.64 mmol). The combined reaction was purified by reverse phase HPLC (C₁₈ 150 mm×40 mm×5 μm column, Mobile Phase A: H₂O/Mobile Phase B: ACN (NH₄OH—NH₄HCO₃), 3 to 43% of Mobile Phase B over 9 min, 100% of Mobile Phase B hold 2 min, flow rate: 60 mL/min) and lyophilized to provide 3 (0.467 g, 46.7% yield). (LC/MS) m/z (M+H)⁺=519.3. ¹H NMR (400 MHz, CDCl₃) δ 8.32 (d, 1H), 7.62 (dd, 1H), 7.35 (d, 2H), 7.31-7.27 (m, 2H), 7.09 (t, 1H), 6.77 (d, 1H), 6.31 (s, 1H), 4.75 (s, 2H), 4.55 (d, 2H), 4.15 (t, 2H), 4.03 (q, 2H), 3.08 (s, 6H), 3.03 (s, 6H), 2.81-2.74 (m, 2H), 1.40 (t, 3H).

### Example 4

### 5-(4-(Dimethylcarbamoyl)phenyl)-1-methyl-N-(4-(methylcarbamoyl)benzyl)-4,5,6,7-tetrahydro-1H-pyrazolo[4,3-c]pyridine-3-carboxamide (4)

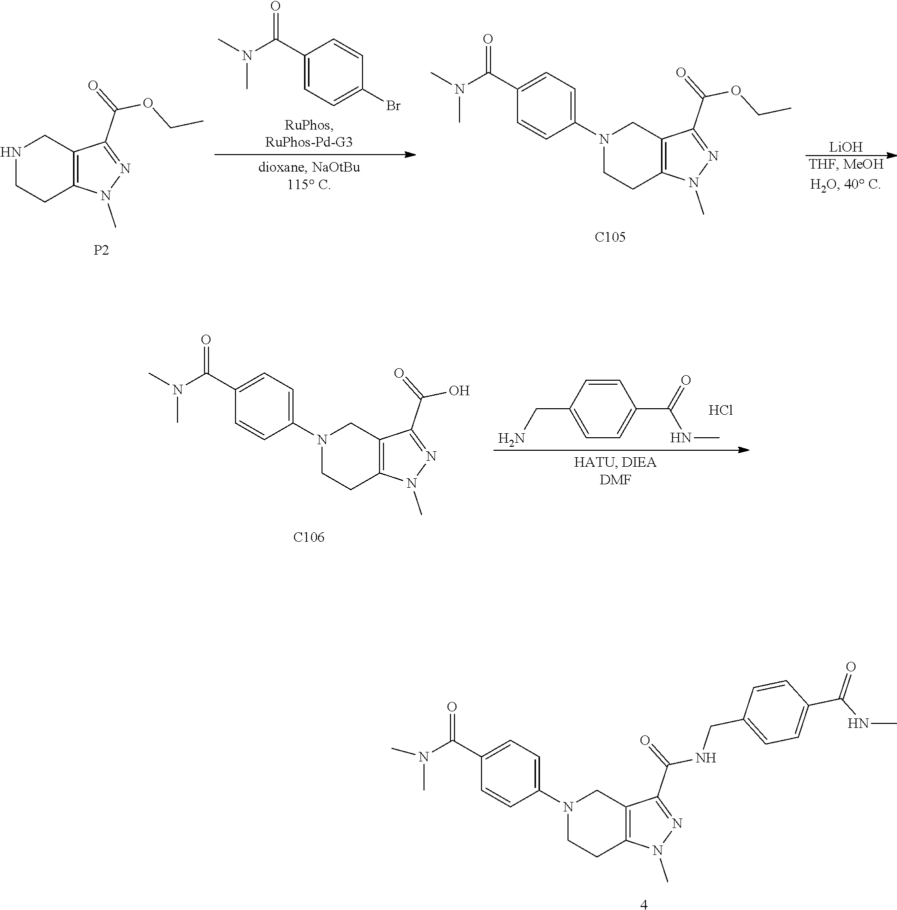

### Step 1. Preparation of ethyl 5-(4-(dimethylcarbamoyl)phenyl)-1-methyl-4,5,6,7-tetrahydro-1H-pyrazolo[4,3-c]pyridine-3-carboxylate (C105)

The reaction was conducted in two batches and then combined for purification. To a solution of P4 (50.0 mg, 0.239 mmol) and 4-bromo-N,N-dimethylbenzamide (CAS: 18469-37-9, 54.5 mg, 0.239 mmol) in dioxane (3.0 mL) was added RuPhos (11.2 mg, 0.0239 mmol), RuPhos Pd G3 (20.0 mg, 0.0239 mmol) and NaOtBu (45.9 mg, 0.478 mmol) at room temperature to form the first batch. The reaction mixture of the first batch was degassed with nitrogen for 1 min and heated at 115° C. for 20 h.

The first batch's reaction mixture was combined with the second batch of the same reaction utilizing P4 (0.400 g, 1.91 mmol). The combined reaction was purified by HPLC (C₁₈ 150 mm×30 mm×5 μm column, Mobile Phase A: H₂O/Mobile Phase B: ACN (0.05% NH₄OH—NH₄HCO₃), 6 to 46% of Mobile Phase B over 9 min, 100% of Mobile Phase B hold 2 min, flow rate: 30 mL/min) and lyophilized to provide C105 (50.0 mg, 6.53% yield) as a white solid. (LC/MS) m/z (M+H)⁺=357.2.

### Step 2. Preparation of 5-(4-(dimethylcarbamoyl)phenyl)-1-methyl-4,5,6,7-tetrahydro-1H-pyrazolo[4,3-c]pyridine-3-carboxylic acid hydrochloride (C106)

To a solution of C105 (50.0 mg, 0.140 mmol) in THF (3.0 mL) and H₂O (0.5 mL) was added LiOH (17.7 mg, 0.421 mmol) at room temperature and then stirred at 40° C. for 16 h. The reaction was concentrated in vacuo, washed with H₂O (2×10 mL), and then acidified with 2M HCl until pH=5-6; the resulting solid was filtered to provide C106 (46.0 mg, 89.8% yield) as a white solid. This was used in the next step without further purification. (LC/MS) m/z (M+H)⁺=329.2.

### Step 3. Preparation of 5-(4-(dimethylcarbamoyl)phenyl)-1-methyl-N-(4-(methylcarbamoyl)benzyl)-4,5,6,7-tetrahydro-1H-pyrazolo[4,3-c]pyridine-3-carboxamide (4)

To a solution of C106 (46.0 mg, 0.140 mmol) in DMF (5.0 mL) was added HATU (79.9 mg, 0.210 mmol), DIEA (54.3 mg, 0.420 mmol), and 4-(aminomethyl)-N-methylbenzamide hydrochloride (CAS: 1158467-80-1, 28.1 mg, 0.140 mmol). The reaction was stirred at room temperature for 12 h. The residue was purified by reverse phase HPLC (C₁₈ 150 mm×30 mm×5 μm, Mobile Phase A: H₂O/Mobile Phase B: ACN (0.05% formic acid), 8 to 33% of Mobile Phase B over 9 min, 100% of Mobile Phase B hold 2 min, flow rate: 30 mL/min) and lyophilized to provide an impure 4 (25.0 mg) as a white solid. The solid was re-purified by reverse phase HPLC (C₁₈ 150 mm×30 mm×5 μm, Mobile Phase A: H₂O/Mobile Phase B: ACN (0.05% NH₄OH—NH₄HCO₃), 4 to 44% of Mobile Phase B over 9 min, 100% of Mobile Phase B hold 2 min, flow rate: 30 mL/min) and lyophilized to provide 4 (12.8 mg, 19.3% yield) as a white solid. (LC/MS) m/z (M+H)⁺=475.2. ¹H NMR (400 MHz, CD₃OD) δ 7.80-7.74 (m, 2H), 7.46-7.42 (m, 2H), 7.38-7.33 (m, 2H), 7.08-7.02 (m, 2H), 4.60 (s, 2H), 4.50 (s, 2H), 3.80 (s, 3H), 3.74 (t, 2H), 3.06 (s, 6H), 2.91 (s, 3H), 2.84 (t, 2H).

### Example 5

### 7-(5-(Dimethylcarbamoyl)pyridin-2-yl)-N-(4-(3,3-dimethylureido)benzyl)-3-methyl-5,6,7,8-tetrahydroimidazo[1,5-a]pyrazine-1-carboxamide (5)

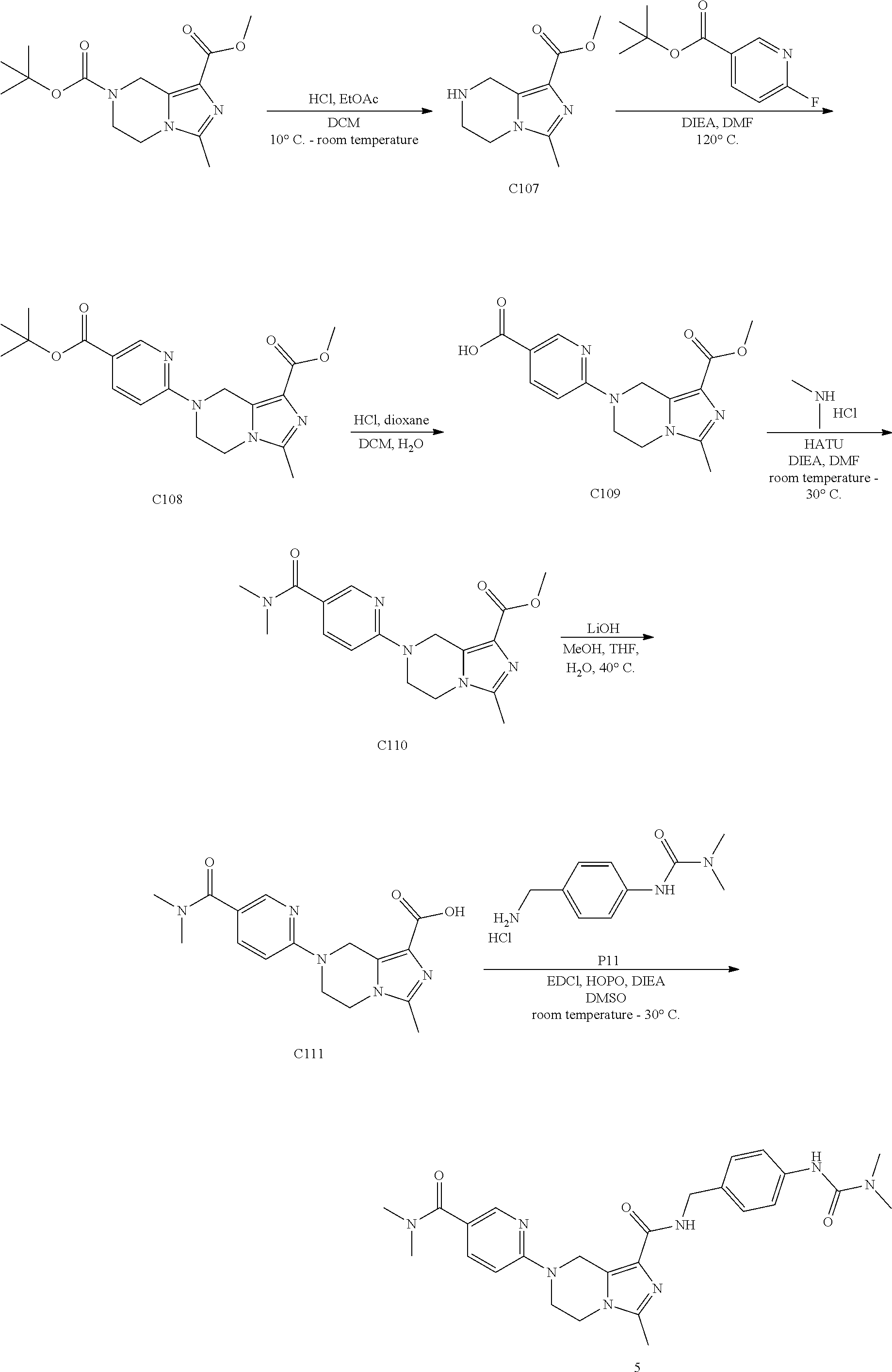

### Step 1. Preparation of methyl 3-methyl-5,6,7,8-tetrahydroimidazo[1,5-a]pyrazine-1-carboxylate hydrochloride (C107)

To a solution of 7-(tert-butyl) 1-methyl 3-methyl-5,6-dihydroimidazo[1,5-a]pyrazine-1,7(8H)-dicarboxylate (CAS: 1359655-89-2, 0.190 g, 0.643 mmol) in DCM (5.0 mL) was added 2M HCl in EtOAc (5.0 mL) at 10° C. The reaction mixture was stirred at room temperature for 16 h. The resulting white suspension was concentrated in vacuo to provide the C107 (0.149 g, crude) as a white solid. This was used in the next step without further purification. ¹H NMR (400 MHz, CD₃OD) δ 4.80 (s, 2H), 4.51 (t, 2H), 4.00 (s, 3H), 3.84 (t, 2H), 2.71 (s, 3H).

### Step 2. Preparation of methyl 7-(5-(tert-butoxycarbonyl)pyridin-2-yl)-3-methyl-5,6,7,8-tetrahydroimidazo[1,5-a]pyrazine-1-carboxylate (C108)

A mixture of C107 (90.0 mg, 0.388 mmol), tert-butyl 6-fluoronicotinate (CAS: 676560-01-3, 152 mg, 0.777 mmol), and DIEA (251 mg, 1.94 mmol) in DMF (4.0 mL) was stirred at 120° C. for 5 h. The light brown reaction mixture was concentrated in vacuo, and the residue was purified by column chromatography (silica gel, 0-100% EtOAc:PE) to provide C108 (87.0 mg, 60.0% yield) as an off-white solid. (LC/MS) m/z (M+H)⁺=373.1. ¹H NMR (400 MHz, CDCl₃) δ 8.80-8.78 (m, 1H), 8.08 (dd, 1H), 6.75 (d, 1H), 5.00 (s, 2H), 4.31 (t, 2H), 4.02 (t, 2H), 3.93 (s, 3H), 2.43 (s, 3H), 1.58 (s, 9H).

### Step 3. Preparation of 6-(1-(methoxycarbonyl)-3-methyl-5,6-dihydroimidazo[1,5-a]pyrazin-7(8H)-yl)nicotinic acid hydrochloride (C109)

To a solution of C108 (87.0 mg, 0.234 mmol) in DCM (2.0 mL) and 2M HCl in dioxane (2.0 mL) was added H₂O (0.1 mL), then the reaction was stirred for 6 h at room temperature. The light-yellow reaction was concentrated in vacuo and then dried under high vacuum for 3 h to provide C109 (73.9 mg, crude) as a light brown residue. This was used in the next step without further purification. (LC/MS) m/z (M+H)⁺=317.1.

### Step 4. Preparation of methyl 7-(5-(dimethylcarbamoyl)pyridin-2-yl)-3-methyl-5,6,7,8-tetrahydroimidazo[1,5-a]pyrazine-1-carboxylate (C110)

To a solution of C109 (73.0 mg, 0.231 mmol) in DMF (5.0 mL) was added HATU (0.132 g, 0.346 mmol), DIEA (0.149 g, 1.15 mmol), and dimethylamine hydrochloride (CAS: 506-59-2; 28.2 mg, 0.346 mmol) at room temperature. The mixture was stirred at room temperature for 4 h, then heated to 30° C. and stirred for 16 h. The reaction mixture was concentrated in vacuo then the residue was purified by column chromatography (silica gel, 0-100% PE:EtOAc to 3.3% MeOH:DCM to provide C110 (71.0 mg, 78.9% yield) as an off-white solid.

### Step 5. Preparation of 7-(5-(dimethylcarbamoyl)pyridin-2-yl)-3-methyl-5,6,7,8-tetrahydroimidazo[1,5-a]pyrazine-1-carboxylic acid (C111)

To a solution of C110 (71.0 mg, 0.207 mmol) in THF (5.0 mL), MeOH (0.5 mL), H₂O (1.0 mL) was added LiOH (15.5 mg, 0.369 mmol) at room temperature. The reaction was stirred at 40° C. for 4 h and then acidified with formic acid to pH=5. The resulting mixture was concentrated in vacuo and then purified by reverse phase HPLC (C₁₈ 150 mm×30 mm×5 μm column, Mobile Phase A: H₂O/Mobile Phase B: ACN (0.05% formic acid), 0 to 16% of Mobile Phase B over 9 min, 100% of Mobile Phase B hold 2 min, flow rate: 30 mL/min) and lyophilized to provide C111 (41.0 mg, 60.2% yield) as a white solid. (LC/MS) m/z (M+H)⁺=330.1.

### Step 6. Preparation of 7-(5-(dimethylcarbamoyl)pyridin-2-yl)-N-(4-(3,3-dimethylureido)benzyl)-3-methyl-5,6,7,8-tetrahydroimidazo[1,5-a]pyrazine-1-carboxamide (5)

A mixture of C111 (40.0 mg, 0.121 mmol), HOPO (40.5 mg, 0.364 mmol), EDCl (55.9 mg, 0.291 mmol), and DIEA (78.5 mg, 0.607 mmol) in DMSO (2.0 mL) was stirred at room temperature for 20 min. P11 (30.7 mg, 0.134 mmol) was added, and the light brown reaction mixture was stirred at 30° C. for 3 h and 30 min. The residue was purified by reverse phase HPLC (C₁₈ 150 mm×30 mm×5 μm column, Mobile Phase A: H₂O/Mobile Phase B: ACN (0.05% formic acid), 0 to 36% of Mobile Phase B over 9 min, 100% of Mobile Phase B hold 2 min, flow rate: 30 mL/min) and lyophilized to provide 5 (28.4 mg, 46.4% yield) as a white solid. (LC/MS) m/z (M+H)⁺=505.4. ¹H NMR (400 MHz, (CD₃)₂SO) δ 8.29-8.26 (m, 1H), 8.21-8.15 (m, 2H), 7.69 (dd, 1H), 7.40-7.35 (m, 2H), 7.17-7.13 (m, 2H), 6.95 (d, 1H), 5.02 (s, 2H), 4.32 (d, 2H), 4.09-3.98 (m, 4H), 2.97 (s, 6H), 2.90 (s, 6H), 2.28 (s, 3H).

### Example 6

### rac-(R)-5-(5-(Dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide (6)

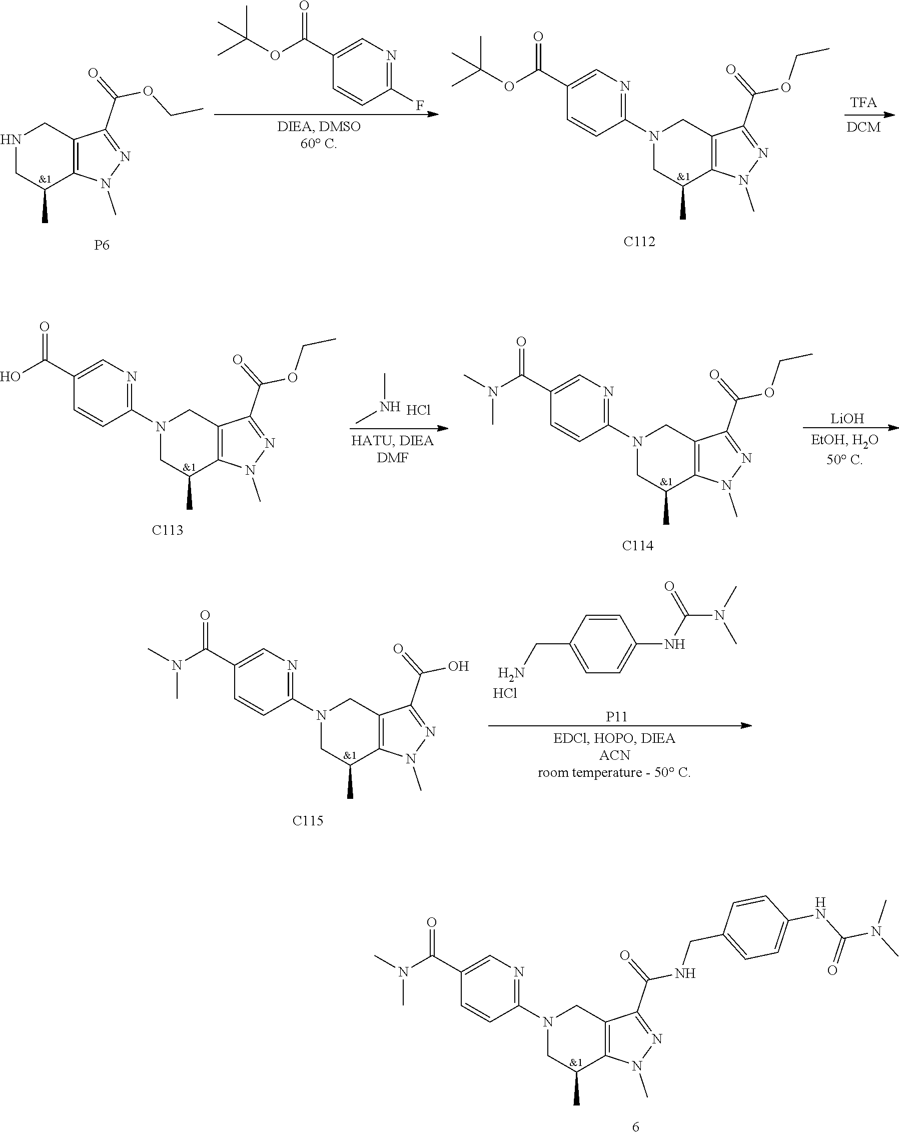

### Step 1: Preparation of rac-ethyl (R)-5-(5-(tert-butoxycarbonyl)pyridin-2-yl)-1,7-dimethyl-4,5,6,7-tetrahydro-1H-pyrazolo[4,3-c]pyridine-3-carboxylate (C112)

To a solution of P6 (0.426 g, 1.64 mmol), tert-butyl 6-fluoronicotinate (CAS: 676560-01-3, 0.356 g, 1.80 mmol) in DMSO (3.4 mL) was added DIEA (1.06 g, 8.20 mmol). The reaction mixture was stirred at 60° C. for 16 h, poured into H₂O (60 mL), and extracted with EtOAc (2×10 mL). The combined organic layers were washed with brine (2×10 mL), dried over Na₂SO₄, filtered, and concentrated in vacuo to give a residue. The residue was purified by column chromatography (silica gel, 0-100% EtOAc:heptane) to provide C112 (0.566 g, 86.2% yield) as a white crystal solid. (LC/MS) m/z (M+H)⁺=401.40. ¹H NMR (400 MHz, CDCl₃) δ 8.77 (dd, 1H), 8.01 (dd, 1H), 6.69 (dd, 1H), 4.95 (d, 1H), 4.72 (dd, 1H), 4.50-4.36 (m, 3H), 3.89 (s, 3H), 3.46 (dd, 1H), 3.13-3.05 (m, 1H), 1.57 (s, 9H), 1.43 (t, 3H), 1.28 (d, 3H).

### Step 2: Preparation of rac-(R)-6-(3-(ethoxycarbonyl)-1,7-dimethyl-1,4,6,7-tetrahydro-5H-pyrazolo[4,3-c]pyridin-5-yl)nicotinic acid (C113)

TFA (2.81 g, 24.7 mmol) was added to a stirred solution of C112 (0.353 g, 0.881 mmol) in DCM (8.0 mL). The reaction was stirred at room temperature for 4 h and then concentrated in vacuo. The resulting gum was sonicated with heptane to yield a solid. The solvent was concentrated in vacuo then the white solid was dissolved in EtOAc and concentrated in vacuo to provide C113 (0.304 g, crude) as an off-white solid. This was used in the next step without further purification. (LC/MS) m/z (M+H)⁺=345.40.

### Step 3: Preparation of rac-ethyl (R)-5-(5-(dimethylcarbamoyl)pyridin-2-yl)-1,7-dimethyl-4,5,6,7-tetrahydro-1H-pyrazolo[4,3-c]pyridine-3-carboxylate (C114)

A mixture of C113 (0.304 g, 0.883 mmol), dimethylamine hydrochloride (CAS: 506-59-2; 86.4 mg, 1.06 mmol), HATU (0.369 g, 0.971 mmol), and DIEA (0.342 g, 2.65 mmol) in DMF (1.2 mL) was stirred at room temperature for 4 h. The reaction mixture was quenched with H₂O (30 mL) and extracted with MeOH in DCM (1:9; 4×100 mL). The combined organic layer was dried over Na₂SO₄ and then concentrated in vacuo. The residue was purified twice by column chromatography. The first purification was by column chromatography (silica gel, 0-100% EtOAc:heptane), and the second purification was by column chromatography (silica gel, 0-15% MeOH:DCM) to provide C114 (0.208 g, 63.4% yield) as a white solid. (LC/MS) m/z (M+H)⁺=372.40. ¹H NMR (400 MHz, CDCl₃) δ 8.32-8.30 (m, 1H), 7.65 (dd, 1H), 6.74 (d, 1H), 4.92 (d, 1H), 4.58 (dd, 1H), 4.46-4.35 (m, 3H), 3.90-3.86 (m, 3H), 3.74-3.64 (m, 1H), 3.46 (dd, 1H), 3.09 (s, 6H), 1.44-1.38 (m, 3H), 1.29 (d, 3H).

### Step 4: Preparation of rac-(R)-5-(5-(dimethylcarbamoyl)pyridin-2-yl)-1,7-dimethyl-4,5,6,7-tetrahydro-1H-pyrazolo[4,3-c]pyridine-3-carboxylic acid (C115)

A flask was charged with C114 (0.106 g, 0.285 mmol), EtOH (2.3 mL), H₂O (0.9 mL), and LiOH (85.5 mg, 1.14 mmol) and then heated to 50° C. The reaction mixture became a solution and was stirred at 50° C. for 1 h and 40 min. The reaction was acidified with 12.1M HCl (0.123 g, 3.37 mmol) then concentrated in vacuo and dried further under high vacuum to provide C115 (0.110 g, crude) as a white solid. This was used in the next step without further purification. (LC/MS) m/z (M+H)⁺=344.40.

### Step 5: Preparation of rac-(R)-5-(5-(dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide (6)

To a solution of C115 (0.110 g, 0.348 mmol) in ACN (1.7 mL) was added HOPO (46.4 mg, 0.417 mmol) and EDCl (0.233 g, 1.22 mmol). The reaction was heated to 50° C. and stirred for 2 h and 20 min. The solution was cooled to room temperature, and DIEA (0.180 g, 1.39 mmol) was added to give a clear yellow solution. P11 (87.9 mg, 0.383 mmol) was added to the reaction and stirred for 1 h at room temperature. The reaction was concentrated in vacuo to give an oil, then dissolved in DCM for purification by column chromatography (silica gel, 0-20% MeOH:DCM) to give an impure 6 (65.0 mg) as a yellow oil. The yellow oil was purified again by reverse phase HPLC (Prep PG-45 MOD10 column, Mobile Phase A: H₂O with 2% ACN/Mobile Phase B: ACN, 5 to 60% of Mobile Phase B over 20 min, 95% of Mobile Phase B hold 5 min, flow rate: 30 mL/min) to provide 6 (34.6 mg, 19.2% yield). (LC/MS) m/z (M+H)⁺=519.2. ¹H NMR (400 MHz, CDCl₃) δ 8.31 (d, 1H), 7.62 (dd, 1H), 7.37-7.32 (m, 2H), 7.28-7.24 (m, 2H), 7.08 (t, 1H), 6.76 (d, 1H), 6.39 (s, 1H), 4.93 (d, 1H), 4.65-4.43 (m, 5H), 3.79 (s, 3H), 3.47 (dd, 1H), 3.08 (s, 6H), 3.02 (s, 6H), 1.28 (d, 3H).

### Examples 7 and 8

### rel-(R or S)-5-(5-(Dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide (7) and (8)

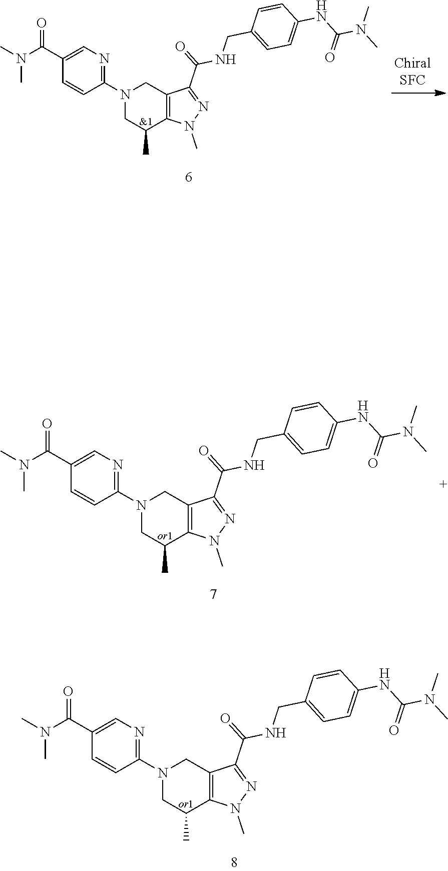

### Step 1. Preparation of rel-(R or S)-5-(5-(dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide (7) and (8)

Chiral SFC purification of compound 6 (44 mg, 0.085 mmol) [Column: Phenomenex Cellulose-1 250 mm×10 mm×5 μm; Mobile phase: 60% CO₂/40% (1:1 MeOH/ACN); flow rate: 25 mL/min; backpressure: 100 bar; column temperature: 40° C.] provided the first eluting isomer as 7 (4.887 min, 22 mg, 50% yield, 100% ee): (LC/MS) m/z (M+H)⁺=519.3 and the second eluting isomer as 8 (5.377 min, 22 mg, 50% yield, 100% ee): (LC/MS) m/z (M+H)⁺=519.4.

### Example 9

### rac-(R)-7-(5-(Dimethylcarbamoyl)pyridin-2-yl)-N-(4-(3,3-dimethylureido)benzyl)-3,5-dimethyl-5,6,7,8-tetrahydroimidazo[1,5-a]pyrazine-1-carboxamide formate (9)

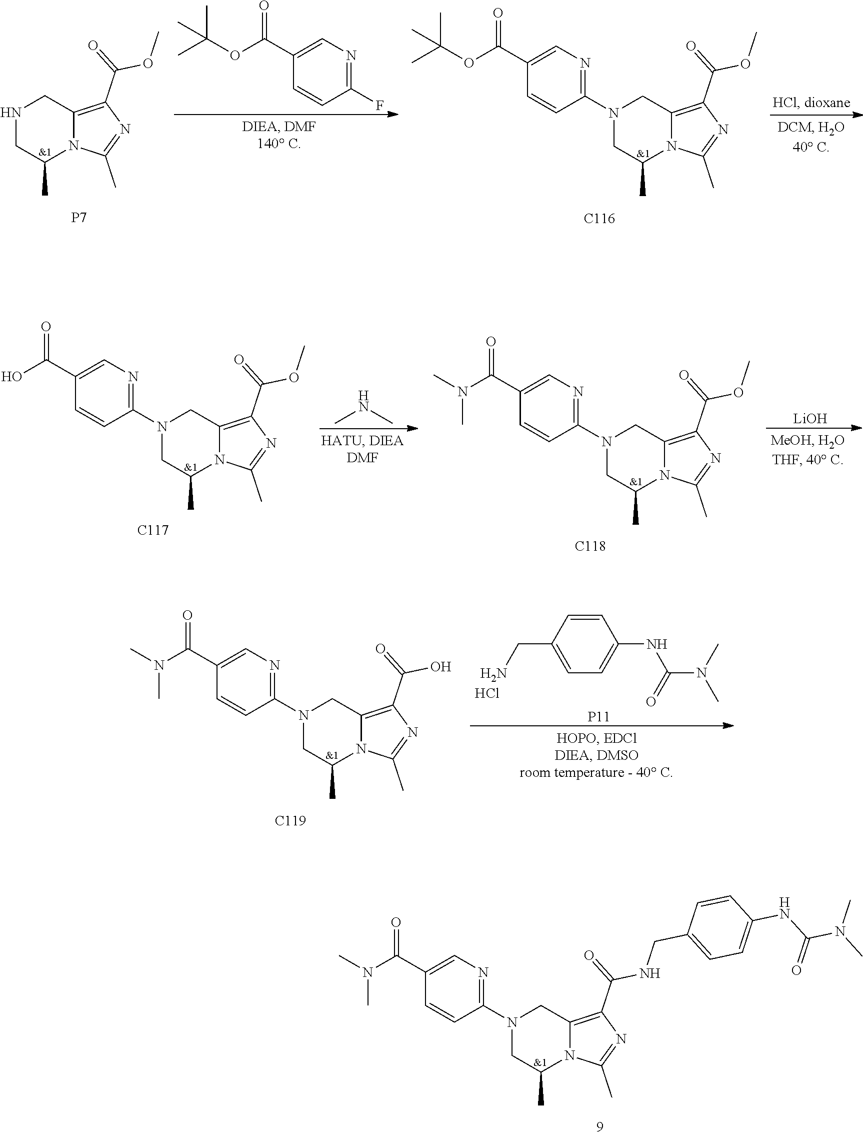

### Step 1: Preparation of rac-methyl (R)-7-(5-(tert-butoxycarbonyl)pyridin-2-yl)-3,5-dimethyl-5,6,7,8-tetrahydroimidazo[1,5-a]pyrazine-1-carboxylate (C116)

A mixture of P7 (0.111 g, 0.452 mmol), tert-butyl 6-fluoronicotinate (CAS: 676560-01-3, 0.223 g, 1.13 mmol), and DIEA (0.292 g, 2.26 mmol) in DMF (5.0 mL) was stirred at 140° C. for 2 h. The light brown reaction mixture was concentrated in vacuo. The residue was purified by column chromatography (silica gel, 0-100% EtOAc:PE then 2% MeOH:EtOAc) to provide C116 (45.0 mg, 25.7% yield) as a light brown solid. (LC/MS) m/z (M+H)⁺=387.1.

### Step 2. Preparation of rac-(R)-6-(1-(methoxycarbonyl)-3,5-dimethyl-5,6-dihydroimidazo[1,5-a]pyrazin-7(8H)-yl)nicotinic acid hydrochloride (C117)

To a solution of C116 (45.0 mg, 0.116 mmol) in DCM (3.0 mL) and 2M HCl in dioxane (10.0 mL) was added H₂O (0.02 mL) at room temperature. The reaction was stirred at 40° C. for 2 h and then concentrated in vacuo to provide C117 (42.7 mg, crude) as an off-white solid. This was used in the next step without further purification. (LC/MS) m/z (M+H)⁺=331.1.

### Step 3. Preparation of rac-methyl (R)-7-(5-(dimethylcarbamoyl)pyridin-2-yl)-3,5-dimethyl-5,6,7,8-tetrahydroimidazo[1,5-a]pyrazine-1-carboxylate (C118)

The reaction was conducted in three batches and then combined for purification. To a solution of C117 (53.0 mg, 0.144 mmol) in DMF (3.0 mL) was added HATU (82.4 mg, 0.217 mmol), DIEA (93.4 mg, 0.722 mmol) and dimethylamine hydrochloride (CAS: 506-59-2; 17.7 mg, 0.217 mmol) at room temperature and then heated to 40° C. and stirred for 16 h. The light brown reaction of the first batch was combined with the second batch of the same reaction utilizing C117 (42.0 mg, 0.116 mmol) and the third batch of the same reaction utilizing C117 (70.0 mg, 0.191 mmol). The combined reaction was concentrated in vacuo then purified by column chromatography (silica gel; 0-100% EtOAc:PE then 2.5% MeOH:EtOAc) to provide C118 (75.0 mg, 41.9% yield) as a light brown solid. (LC/MS) m/z (M+H)⁺=358.1.

### Step 4. Preparation of rac-(R)-7-(5-(dimethylcarbamoyl)pyridin-2-yl)-3,5-dimethyl-5,6,7,8-tetrahydroimidazo[1,5-a]pyrazine-1-carboxylic acid (C119)

The reaction was conducted in two batches and then combined for purification. To a solution of C118 (75.0 mg, 0.210 mmol) in THF (4.0 mL), MeOH (1.0 mL), and H₂O (2.0 mL) was added LiOH (17.6 mg, 0.420 mmol) at room temperature, which was then heated at 40° C. for 2 h. Another portion of LiOH (8.8 mg, 0.21 mmol) was added and stirred at 40° C. for 3 h.

The reaction of the first batch was combined with the second batch of the same reaction utilizing C118 (33.0 mg, 0.0923 mmol), then acidified with formic acid to pH=˜5 and concentrated in vacuo. The residue was purified by reverse phase HPLC (C₁₈ 150 mm×30 mm×5 μm column, Mobile Phase A: H₂O/Mobile Phase B: ACN (0.05% formic acid), 0 to 25% of Mobile Phase B over 9 min, 100% of Mobile Phase B hold 2 min, flow rate: 30 mL/min) and lyophilized to provide C119 (59.0 mg, 56.7% yield) as a light brown solid. (LC/MS) m/z (M+H)⁺=344.1.

### Step 5. Preparation of rac-(R)-7-(5-(dimethylcarbamoyl)pyridin-2-yl)-N-(4-(3,3-dimethylureido)benzyl)-3,5-dimethyl-5,6,7,8-tetrahydroimidazo[1,5-a]pyrazine-1-carboxamide formate (9)

A mixture of C119 (59.0 mg, 0.172 mmol), HOPO (57.3 mg, 0.515 mmol), EDCl (79.1 mg, 0.412 mmol) and DIEA (111 mg, 0.859 mmol) in DMSO (3.0 mL) was stirred for 30 min at 40° C., then P11 (59.2 mg, 0.258 mmol) was added at room temperature. The light brown reaction mixture was stirred at 40° C. for 3 h and 30 min then the residue was purified by reverse phase HPLC (C₁₈ 150 mm×30 mm×5 μm column, Mobile Phase A: H₂O/Mobile Phase B: ACN (0.05% formic acid), 0 to 39% of Mobile Phase B over 9 min, 100% of Mobile Phase B hold 2 min, flow rate: 30 mL/min) and lyophilized to provide 9 (58.2 mg, 43.2% yield) as a white solid. (LC/MS) m/z (M+H)⁺=519.5. ¹H NMR (400 MHz, CD₃OD) δ 8.31 (d, 1H), 7.74 (dd, 1H), 7.36-7.30 (m, 2H), 7.26 (d, 2H), 6.93 (d, 1H), 5.39 (d, 1H), 4.67-4.41 (m, 5H), 3.41 (dd, 1H), 3.10 (s, 6H), 3.04-2.99 (m, 6H), 2.41 (s, 3H), 1.40 (d, 3H).

### Example 10

### 1-Cyclopropyl-5-(5-(dimethylcarbamoyl)pyridin-2-yl)-N-(4-(3,3-dimethylureido)benzyl)-4,5,6,7-tetrahydro-1H-pyrazolo[4,3-c]pyridine-3-carboxamide trifluoroacetate (10)

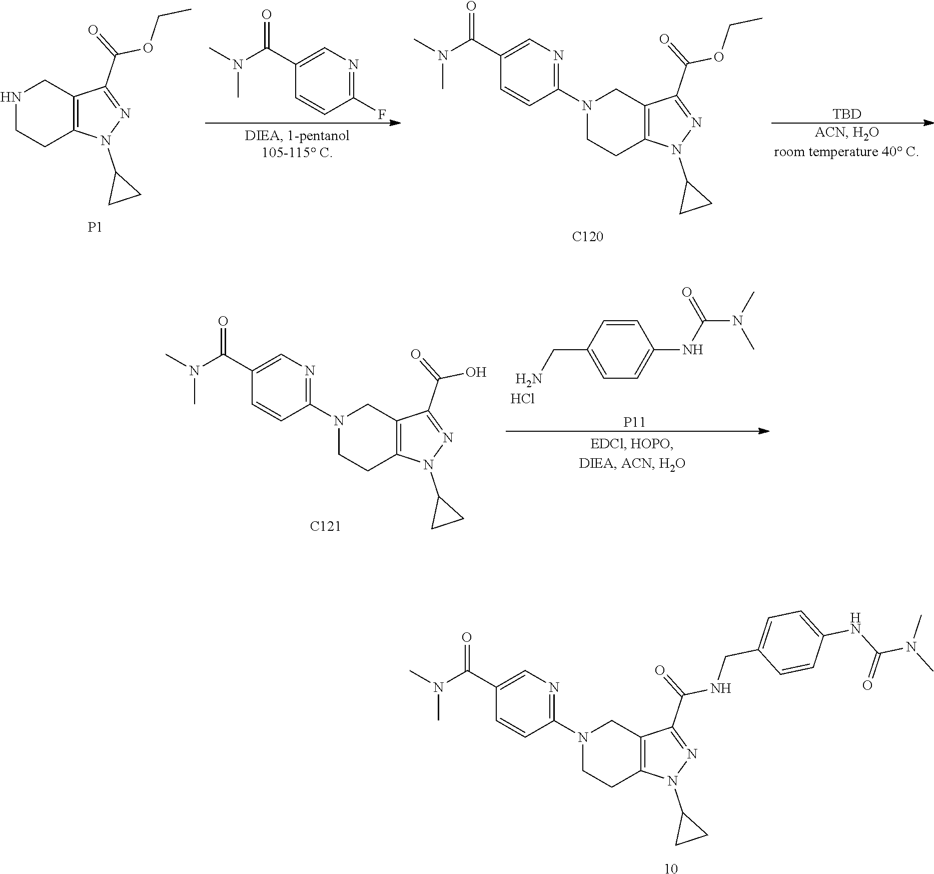

### Step 1. Preparation of ethyl 1-cyclopropyl-5-(5-(dimethylcarbamoyl)pyridin-2-yl)-4,5,6,7-tetrahydro-1H-pyrazolo[4,3-c]pyridine-3-carboxylate (C120)

To a solution of P1 (0.070 g, 0.26 mmol) in 1-pentanol (1 mL) and DIEA (0.20 g, 1.5 mmol) was added 6-fluoro-N,N-dimethylnicotinamide (CAS: 1032251-82-3; 65 mg, 0.39 mmol).

The reaction mixture was heated at 105° C. for 24 h, then the temperature was increased to 115° C. and stirred for 23 h. The reaction mixture was concentrated in vacuo and then dissolved with DCM. The solution was purified by column chromatography (silica gel, 0-5% MeOH:DCM) to provide C120 (0.10 g, crude) as a yellow oil. The oil was used directly in the next step without further purification.

### Step 2. Preparation of 1-cyclopropyl-5-(5-(dimethylcarbamoyl)pyridin-2-yl)-4,5,6,7-tetrahydro-1H-pyrazolo[4,3-c]pyridine-3-carboxylic acid (C121)

To a solution of C120 (99 mg, 0.26 mmol) in ACN (1 mL) and H₂O (93 μL) was added TBD (0.11 g, 0.77 mmol) and then stirred for 40 min at room temperature followed by 40° C. for 2 h. The reaction was then cooled to room temperature, and 12.1M HCl (24 mg, 0.65 mmol) was added. The acidic reaction mixture was concentrated in vacuo to provide C121 (0.090 g, crude) as a yellow oil. The oil was used directly in the next step without further purification. (LC/MS) m/z (M+H)⁺=356.2.

### Step 3. Preparation of 1-cyclopropyl-5-(5-(dimethylcarbamoyl)pyridin-2-yl)-N-(4-(3,3-dimethylureido)benzyl)-4,5,6,7-tetrahydro-1H-pyrazolo[4,3-c]pyridine-3-carboxamide trifluoroacetate (10)

To a solution of C121 (0.090 g, 0.25 mmol) in ACN (1.0 mL) and H₂O (0.2 mL) was added HOPO (34 mg, 0.30 mmol) and EDCl (78 mg, 0.41 mmol). To the reaction mixture was added an additional portion of EDCl (78 mg, 0.41 mmol) and then was stirred at room temperature for 20 min before DIEA (98 mg, 0.76 mmol) and P11 (0.070 g, 0.30 mmol) were added. The suspension was stirred at room temperature for 30 min and then was concentrated in vacuo. The residue was diluted with DMSO then acidified with TFA and purified with reverse phase HPLC (Sunfire C₁₈ 100 mm×19 mm×5 μm column, Mobile Phase A: H₂O (0.05% TFA)/Mobile Phase B: ACN (0.05% TFA), 5 to 95% of Mobile Phase B over 9 min, 95% of Mobile Phase B hold 1 min, flow rate=25 mL/min) and lyophilized to provide 10 (24 mg, 18% yield). (LC/MS) m/z (M+H)⁺=531.5. ¹H NMR (600 MHz, (CD₃)₂SO) δ 8.46 (t, 1H), 8.22-8.21 (m, 2H), 7.68 (dd, 1H), 7.40-7.34 (m, 2H), 7.15 (d, 2H), 6.96 (d, 1H), 4.75 (s, 2H), 4.32 (d, 2H), 3.96 (t, 2H), 3.56 (tt, 1H), 2.97 (s, 6H), 2.91-2.86 (m, 8H), 1.12-1.05 (m, 2H), 1.01-0.95 (m, 2H).

### Example 12

### N-(4-(3,3-Dimethylureido)benzyl)-1-methyl-5-(5-(1-methyl-1H-imidazol-2-yl)pyridin-2-yl)-4,5,6,7-tetrahydro-1H-pyrazolo[4,3-c]pyridine-3-carboxamide (12)

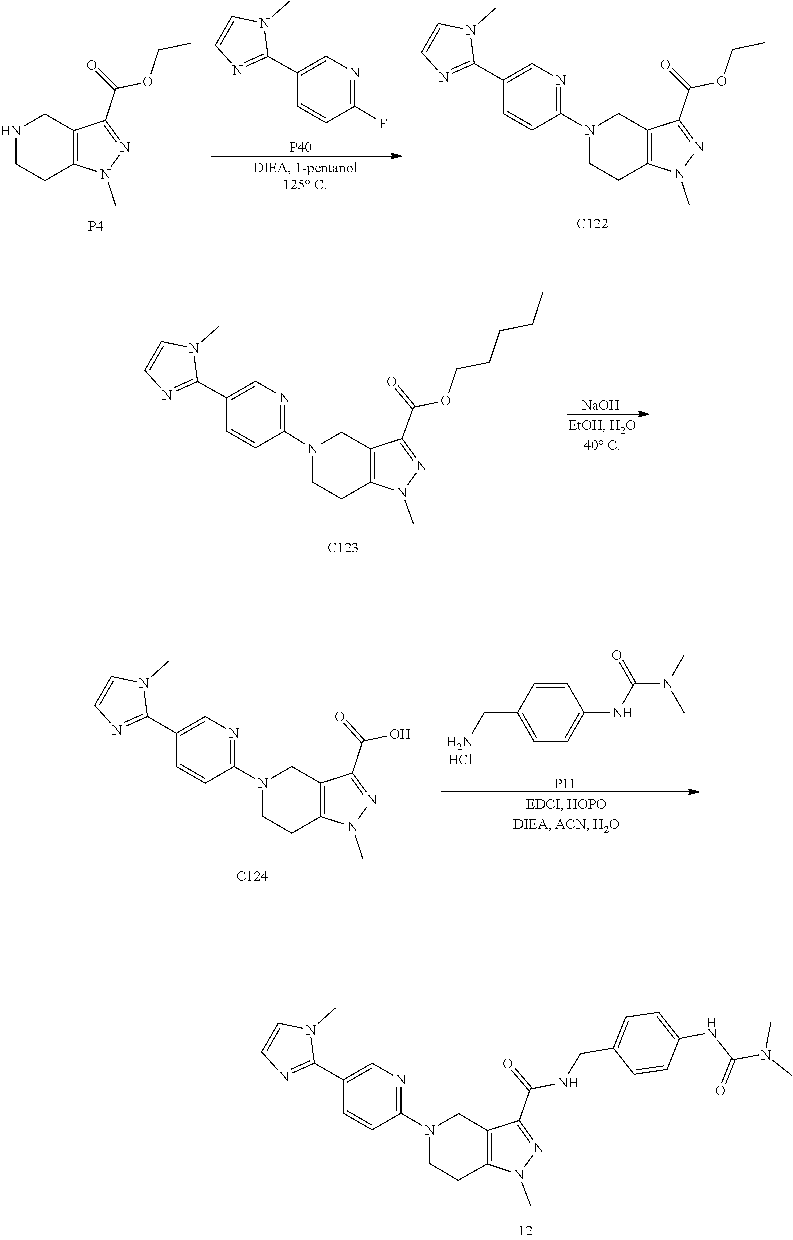

### Step 1. Preparation of ethyl 1-methyl-5-(5-(1-methyl-1H-imidazol-2-yl)pyridin-2-yl)-4,5,6,7-tetrahydro-1H-pyrazolo[4,3-c]pyridine-3-carboxylate (C122) and pentyl 1-methyl-5-(5-(1-methyl-1H-imidazol-2-yl)pyridin-2-yl)-4,5,6,7-tetrahydro-1H-pyrazolo[4,3-c]pyridine-3-carboxylate (C123)

To a suspension of P4 (0.60 g, 2.4 mmol) in 1-pentanol (5 mL) was added DIEA (1.9 g, 15 mmol) and P40 (0.47 g, 2.7 mmol), then heated at 125° C. for 20 h. The reaction mixture was checked by LCMS which showed 30% of C122 (LC/MS) m/z (M+H)⁺=367.3 and 32% of C123 at (LC/MS) m/z (M+H)⁺=409.3 was formed due to transesterification with 1-pentanol. The suspension was concentrated in vacuo then dissolved in DCM and heptane. The solution was purified by column chromatography (silica gel, 0-10% MeOH:DCM) to form a mixture of C122 and C123 (1.1 g, crude) as a brown oil. The brown oil was used directly in the next step without further purification.

### Step 2. Preparation of 1-methyl-5-(5-(1-methyl-1H-imidazol-2-yl)pyridin-2-yl)-4,5,6,7-tetrahydro-1H-pyrazolo[4,3-c]pyridine-3-carboxylic acid (C124)

To a reaction mixture of C122 and C123 (0.80 g, 2.2 mmol) in EtOH (6 mL) and H₂O (4 mL) was added NaOH (0.35 g, 8.7 mmol) then stirred at 40° C. for 50 min. The suspension was concentrated in vacuo and acidified with 12.1M HCl (0.40 g, 11 mmol) to pH˜1. The acidic reaction mixture was extracted with DCM then the organic layer was concentrated in vacuo to form C124 (0.30 g, crude) as a brown oil. The oil was used directly in the next step without further purification.

### Step 3. Preparation of N-(4-(3,3-dimethylureido)benzyl)-1-methyl-5-(5-(1-methyl-1H-imidazol-2-yl)pyridin-2-yl)-4,5,6,7-tetrahydro-1H-pyrazolo[4,3-c]pyridine-3-carboxamide (12)

To a reaction mixture of C124 (0.30 g, 0.89 mmol) in ACN (3.0 mL) and H₂O (0.5 mL) was added HOPO (0.12 g, 1.1 mmol) and EDCl (0.27 g, 1.4 mmol). The suspension was stirred at room temperature for 35 min before adding DIEA (0.34 g, 2.7 mmol) and P11 (0.24 g, 1.1 mmol). The reaction mixture was stirred at room temperature for 30 min and then was concentrated in vacuo. The residue was split into two portions for purification.

The first portion of the residue was diluted with DMSO and then purified with reverse-phase HPLC (Sunfire C₁₈ 100 mm×19 mm×5 μm column, Mobile Phase A: H₂O (0.05% TFA)/Mobile Phase B: ACN (0.05% TFA), 5 to 95% of Mobile Phase B over 9 min, 95% of Mobile Phase B hold 1 min, flow rate=25 mL/min) and lyophilized to provide 12 (8.5 mg).

The second portion of the residue was diluted with DCM and then purified by column chromatography (silica gel, 0-5% MeOH:DCM) to provide an impure residue. The impure residue was diluted with MeOH then purified by SFC (Princeton Methanesulfamide Column 250 mm×30 mm×5 μm, 70% CO₂/30% MeOH (0.2% 7N NH₃ in MeOH), backpressure=100 bar, temperature=40° C., flow rate=80 mL/min) to provide 12 (62 mg) as a tan solid.

The two purified batches were combined to form 12 (0.070 g, 15% yield) as a solid. (LC/MS) m/z (M+H)⁺=514.4. ¹H NMR (600 MHz, (CD₃)₂SO) δ 8.47 (t, 1H), 8.43 (d, 1H), 8.22 (s, 1H), 7.84 (dd, 1H), 7.42-7.34 (m, 2H), 7.22 (s, 1H), 7.16 (d, 2H), 7.01-6.93 (m, 2H), 4.77 (s, 2H), 4.32 (d, 2H), 3.96 (t, 2H), 3.76 (s, 3H), 3.71 (s, 3H), 2.90 (s, 6H), 2.80 (t, 2H).

### Example 18

### 5-(5-(Dimethylcarbamoyl)pyridin-2-yl)-N-(4-(3,3-dimethylureido)benzyl)-4,5,6,7-tetrahydro-1H-pyrazolo[4,3-c]pyridine-3-carboxamide (18)

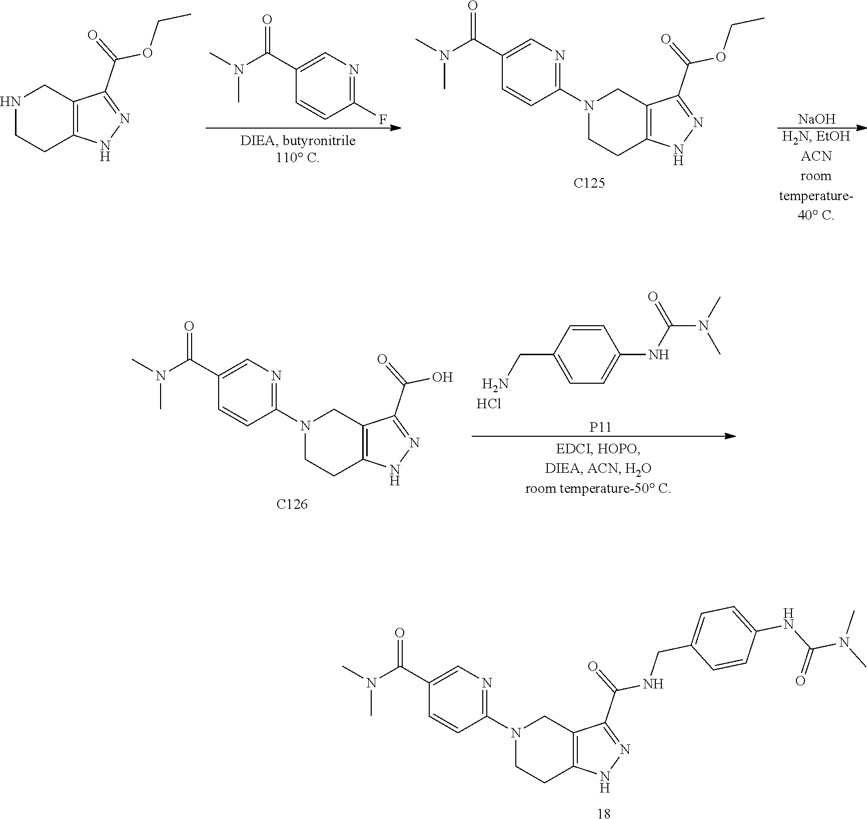

### Step 1. Preparation of ethyl 5-(5-(dimethylcarbamoyl)pyridin-2-yl)-4,5,6,7-tetrahydro-1H-pyrazolo[4,3-c]pyridine-3-carboxylate (C125)

To a reaction mixture of ethyl 4,5,6,7-tetrahydro-1H-pyrazolo[4,3-c]pyridine-3-carboxylate hydrochloride (CAS: 1211512-51-4; 0.380 g, 1.64 mmol) in butyronitrile (10 mL) and DIEA (0.848 g, 6.56 mmol) was added 6-fluoro-N,N-dimethylnicotinamide (CAS: 1032251-82-3, 0.290 g, 1.72 mmol) then stirred at 110° C. for 23 h. The solution was concentrated in vacuo and then redissolved in DCM. The solution was purified by column chromatography (silica gel, 0-7% MeOH:DCM) to provide C125 (0.439 g, 80.0% yield) as a yellow oil. (LC/MS) m/z (M+H)⁺=344.3.

### Step 2. Preparation of 5-(5-(dimethylcarbamoyl)pyridin-2-yl)-4,5,6,7-tetrahydro-1H-pyrazolo[4,3-c]pyridine-3-carboxylic acid (C126)

To a solution of C125 (0.439 g, 1.28 mmol) in ACN (5 mL), H₂O (2 mL), and EtOH (5 mL) was added NaOH (102 mg, 2.56 mmol). The solution was stirred at room temperature for 2 h then heated to 40° C. and stirred for 3 h and 20 min. The reaction solution was acidified with 12.1M HCl (0.140 g, 3.84 mmol) then concentrated in vacuo to provide C126 (0.384 g, crude) as an off-white solid. The solid was used directly in the next step without further purification. (LC/MS) m/z (M+H)⁺=316.3.

### Step 3. Preparation of 5-(5-(dimethylcarbamoyl)pyridin-2-yl)-N-(4-(3,3-dimethylureido)benzyl)-4,5,6,7-tetrahydro-1H-pyrazolo[4,3-c]pyridine-3-carboxamide (18)

To a solution of C126 (0.200 g, 0.634 mmol) in ACN (5 mL) was added HOPO (84.6 mg, 0.761 mmol) and EDCl (0.195 g, 1.01 mmol). The reaction mixture was stirred at 50° C. for 20 min, then H₂O (1 mL), DIEA (0.246 g, 1.90 mmol), and P11 (0.160 g, 0.698 mmol) were added. The reaction mixture was stirred at room temperature for 15 min then concentrated in vacuo. The residue was dissolved in DCM and then purified by column chromatography (silica gel, 0-15% MeOH:DCM) to form an impure 18 (0.386 g).

A portion of the impure 18 (0.0400 g) was purified by reverse phase HPLC (Sunfire C18 100 mm×19 mm×5 μm column, Mobile Phase A: H₂O/Mobile Phase B: ACN (0.05% TFA), 5 to 95% of Mobile Phase B over 9 min, 95% oh Mobile Phase B hold 1 min, flow rate=25 mL/min) and lyophilized to provide 18 (9.90 mg).

The remaining of the portion of the impure 18 (0.346 g) was purified by SFC (Princeton CN 250 mm×30 mm×5 μm, 70% CO₂/25% MeOH (7N NH₃ in MeOH), backpressure=100 bar, temperature=40° C., flow rate=80 mL/min) to provide 18 (55.9 mg).

The two final batches were combined to provide 18 (65.8 mg, 21.1% yield) as a solid. (LC/MS) m/z (M+H)⁺=491.4. ¹H NMR (400 MHz, CD₃OD) δ 8.26 (d, 1H), 7.67 (dd, 1H), 7.35-7.25 (m, 4H), 6.89 (d, 1H), 4.78 (s, 2H), 4.50 (s, 2H), 4.05 (t, 2H), 3.08 (s, 6H), 3.00 (s, 6H), 2.84 (t, 2H).

### Example 23

### N-(4-(3,3-Dimethylureido)benzyl)-1-ethyl-5-(5-(1-methyl-1H-1,2,4-triazol-5-yl)pyridin-2-yl)-4,5,6,7-tetrahydro-1H-pyrazolo[4,3-c]pyridine-3-carboxamide (23)

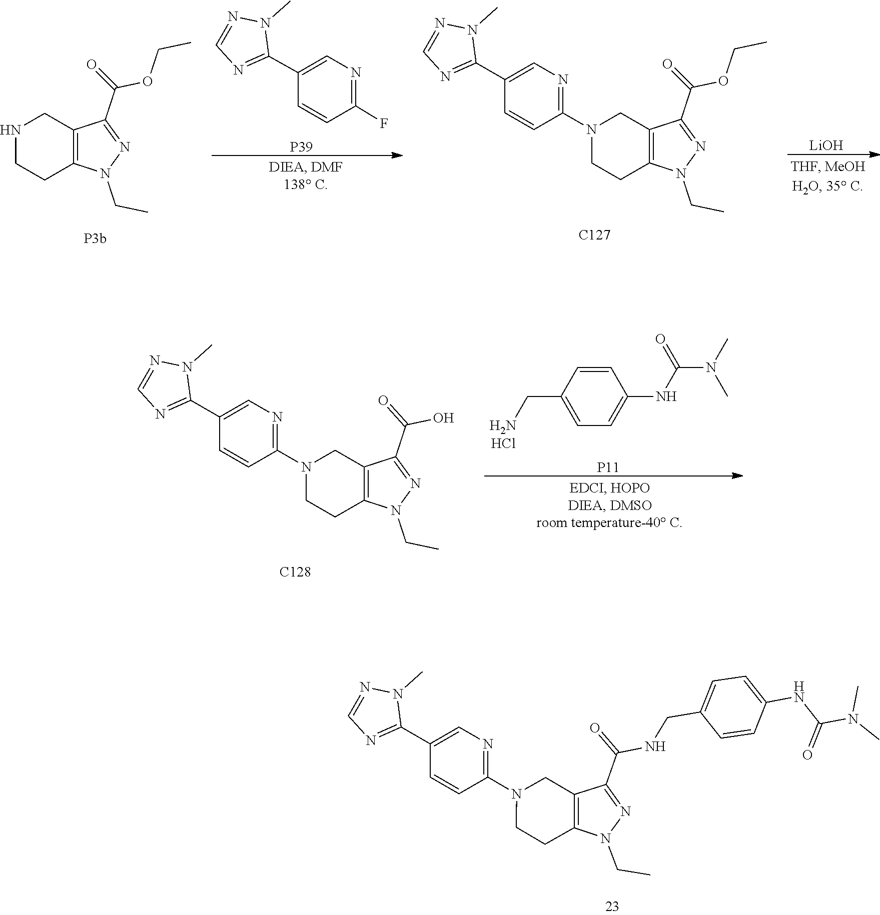

### Step 1. Preparation of ethyl 1-ethyl-5-(5-(1-methyl-1H-1,2,4-triazol-5-yl)pyridin-2-yl)-4,5,6,7-tetrahydro-1H-pyrazolo[4,3-c]pyridine-3-carboxylate (C127)

To a reaction mixture of P3b (0.150 g, 0.842 mmol) and P39 (0.219 g, 0.842 mmol) in DMF (4.2 mL) was added DIEA (0.544 g, 4.21 mmol) and then stirred at 138° C. for 48 h. The suspension was diluted with EtOAc (30 mL), and then the organic layer was washed with H₂O (20 mL) and brine (20 mL). The organic layer was dried with Na₂SO₄ and then concentrated in vacuo. The residue was purified by column chromatography (silica gel, 0-58% THF:PE) to provide C127 (0.200 g, 62.3% yield) as a white solid. (LC/MS) m/z (M+H)⁺=382.2.

### Step 2. Preparation of 1-ethyl-5-(5-(1-methyl-1H-1,2,4-triazol-5-yl)pyridin-2-yl)-4,5,6,7-tetrahydro-1H-pyrazolo[4,3-c]pyridine-3-carboxylic acid (C128)

To a solution of C127 (0.200 g, 0.524 mmol) in THF (2.0 mL), H₂O (0.5 mL), and MeOH (0.5 mL) was added LiOH (66.0 mg, 1.57 mmol) at room temperature. The reaction mixture was heated to 35° C. and stirred for 2 h. The suspension was extracted with EtOAc (2×10 mL), and then the combined organic layer was washed with H₂O (10 mL). The aqueous layer was acidified to pH=3 with citric acid, which caused precipitation to form. The suspension was filtered, and then the filter cake was collected and lyophilized to provide C128 (0.150 g, crude) as a white solid. The solid was used directly in the next step without further purification. (LC/MS) m/z (M+H)⁺=354.2.

### Step 3. Preparation of N-(4-(3,3-dimethylureido)benzyl)-1-ethyl-5-(5-(1-methyl-1H-1,2,4-triazol-5-yl)pyridin-2-yl)-4,5,6,7-tetrahydro-1H-pyrazolo[4,3-c]pyridine-3-carboxamide (23)

The reaction was conducted in two batches and then combined for purification. To a solution of C128 (0.060 g, 0.17 mmol) in DMSO (1.0 mL) was added HOPO (56 mg, 0.50 mmol), DIEA (0.11 g, 0.85 mmol), and EDCl (78 mg, 0.41 mmol). The reaction mixture was stirred at room temperature for 5 min, and then P11 (58 mg, 0.25 mmol) was added to form the first batch. The reaction mixture of the first batch was stirred at 40° C. for 2 h.

A second batch of the same reaction was conducted with C128 (15 mg, 0.043 mmol). The two batches were combined then purified by reverse phase HPLC (Boston Prime C₁₈ 150 mm×30 mm×5 μm column, Mobile Phase A: H₂O/Mobile Phase B: ACN (NH₄OH—NH₄HCO₃), 20 to 50% of Mobile Phase B over 9 min, 100% of Mobile Phase B hold 3 min, flow rate=50 mL/min) and lyophilized to provide 23 (62 mg, 55% yield) as a white solid. (LC/MS) m/z (M+H)⁺=529.3. ¹H NMR (400 MHz, (CD₃)₂SO) δ 8.56 (d, 1H), 8.44 (t, 1H), 8.23 (s, 1H), 7.99-7.92 (m, 2H), 7.45-7.34 (m, 2H), 7.22-7.17 (m, 2H), 7.05 (d, 1H), 4.83 (s, 2H), 4.36 (d, 2H), 4.11 (q, 2H), 4.02 (t, 2H), 3.97 (s, 3H), 2.93 (s, 6H), 2.86 (t, 2H), 1.36 (t, 3H).

### Example 26

### rac-(R)-5-(4-(Dimethylcarbamoyl)phenyl)-N-(4-(3,3-dimethylureido)benzyl)-1-methyl-4,5,6,7-tetrahydro-1H-indazole-3-carboxamide (26)

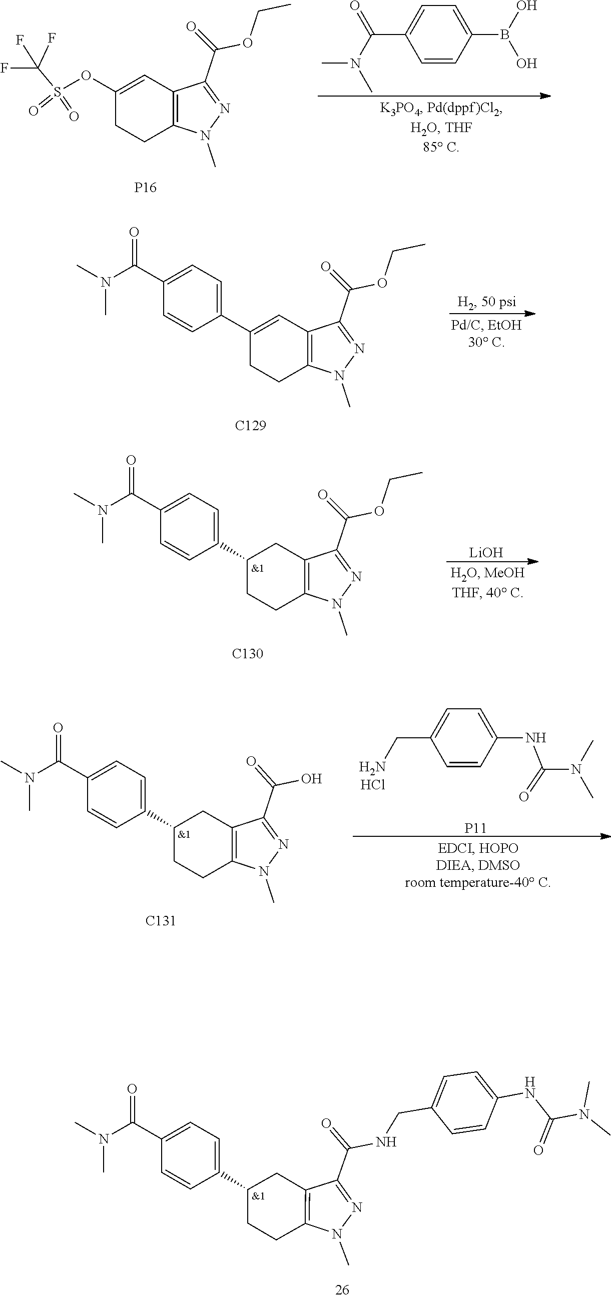

### Step 1. Preparation of ethyl 5-(4-(dimethylcarbamoyl)phenyl)-1-methyl-6,7-dihydro-1H-indazole-3-carboxylate (C129)

Under nitrogen gas, to a mixture of P16 (0.500 g, 1.41 mmol) and (4-(dimethylcarbamoyl)phenyl)boronic acid (0.409 g, 2.12 mmol) in H₂O (1 mL) and THF (10 mL) was added Pd(dppf)Cl₂ (0.103 g, 0.141 mmol) and K₃PO₄ (0.899 g, 4.23 mmol). The reaction mixture was stirred at 85° C. for 16 h and then concentrated in vacuo. The residue was purified by column chromatography (silica gel, 0-62% THF:PE) to provide C129 (0.470 g, 94.2% yield) as a pink solid. (LC/MS) m/z (M+H)⁺=354.1. ¹H NMR (400 MHz, (CD₃)₂SO) δ 7.62-7.55 (m, 2H), 7.45-7.39 (m, 2H), 7.15-7.12 (m, 1H), 4.33-4.24 (m, 2H), 3.85-3.82 (m, 3H), 3.76-3.54 (m, 2H), 3.01-2.94 (m, 6H), 2.93-2.85 (m, 2H), 1.31 (t, 3H).

### Step 2. Preparation of rac-ethyl (R)-5-(4-(dimethylcarbamoyl)phenyl)-1-methyl-4,5,6,7-tetrahydro-1H-indazole-3-carboxylate (C130)

A solution of C129 (0.050 g, 0.14 mmol) was added in a thick-walled pressure vial in EtOH (5 mL). Pd/C (45 mg, 0.042 mmol) was added to the solution. The vial was evacuated under vacuum then purged with H₂ gas. The reaction mixture was stirred under H₂ atmosphere (50 psi) at 30° C. for 16 h then concentrated in vacuo to provide C130 (45 mg, 89% yield) as a yellow oil. ¹H NMR (400 MHz, CDCl₃) δ 7.39-7.35 (m, 2H), 7.30-7.26 (m, 2H), 4.37 (q, 2H), 3.83 (s, 3H), 3.19 (dd, 1H), 3.14-2.98 (m, 6H), 2.97-2.90 (m, 1H), 2.81-2.61 (m, 3H), 2.20-2.12 (m, 1H), 2.05-1.92 (m, 1H), 1.37 (t, 3H).

### Step 3. Preparation of rac-(R)-5-(4-(dimethylcarbamoyl)phenyl)-1-methyl-4,5,6,7-tetrahydro-1H-indazole-3-carboxylic acid (C131)

To a solution of C130 (45 mg, 0.13 mmol) in THF (1.00 mL), H₂O (0.25 mL), and MeOH (0.25 mL) was added LiOH (16 mg, 0.38 mmol). The reaction mixture was stirred at 40° C. for 2 h, diluted with EtOAc (2×10 mL), and washed with H₂O (10 mL). The aqueous phase was separated and adjusted with citric acid to pH˜3, then extracted with DCM (5×50 mL). The combined organic layer was dried with Na₂SO₄ and concentrated in vacuo to provide C131 (35 mg, 84% yield) as a brown solid. (LC/MS) m/z (M+H)⁺=328.1.

### Step 4. Preparation of rac-(R)-5-(4-(dimethylcarbamoyl)phenyl)-N-(4-(3,3-dimethylureido)benzyl)-1-methyl-4,5,6,7-tetrahydro-1H-indazole-3-carboxamide (26)

To a solution of C131 (35 mg, 0.11 mmol) in DMSO (1 mL) was added HOPO (35 mg, 0.31 mmol), EDCl (49 mg, 0.26 mmol), and DIEA (69 mg, 0.53 mmol). The reaction mixture was stirred at room temperature for 5 min, and then P11 (37 mg, 0.16 mmol) was added. The suspension was stirred at 40° C. for 1 h then was purified by reverse phase HPLC (C₁₈ 150 mm×mm×5 μm column, Mobile Phase A: H₂O (NH₄OH—NH₄HCO₃)/Mobile Phase B: ACN, 14 to 44% of Mobile Phase B over 10 min, 100% of Mobile Phase B hold 3 min, flow rate=50 mL/min) and lyophilized to provide 26 (11 mg, 20.% yield) as a white solid. (LC/MS) m/z (M+H)⁺=503.4. ¹H NMR (400 MHz, (CD₃)₂SO) δ 8.31 (t, 1H), 8.22 (s, 1H), 7.42-7.32 (m, 6H), 7.17-7.11 (m, 2H), 4.31 (d, 2H), 3.76 (s, 3H), 3.14-3.04 (m, 1H), 3.00-2.89 (m, 12H), 2.85-2.58 (m, 4H), 2.12-1.88 (m, 2H).

### Example 30

### N-(4-(3,3-Dimethylureido)benzyl)-1-ethyl-5-(5-(1-methyl-1H-imidazol-2-yl)pyridin-2-yl)-4,5,6,7-tetrahydro-1H-pyrazolo[4,3-c]pyridine-3-carboxamide (30)

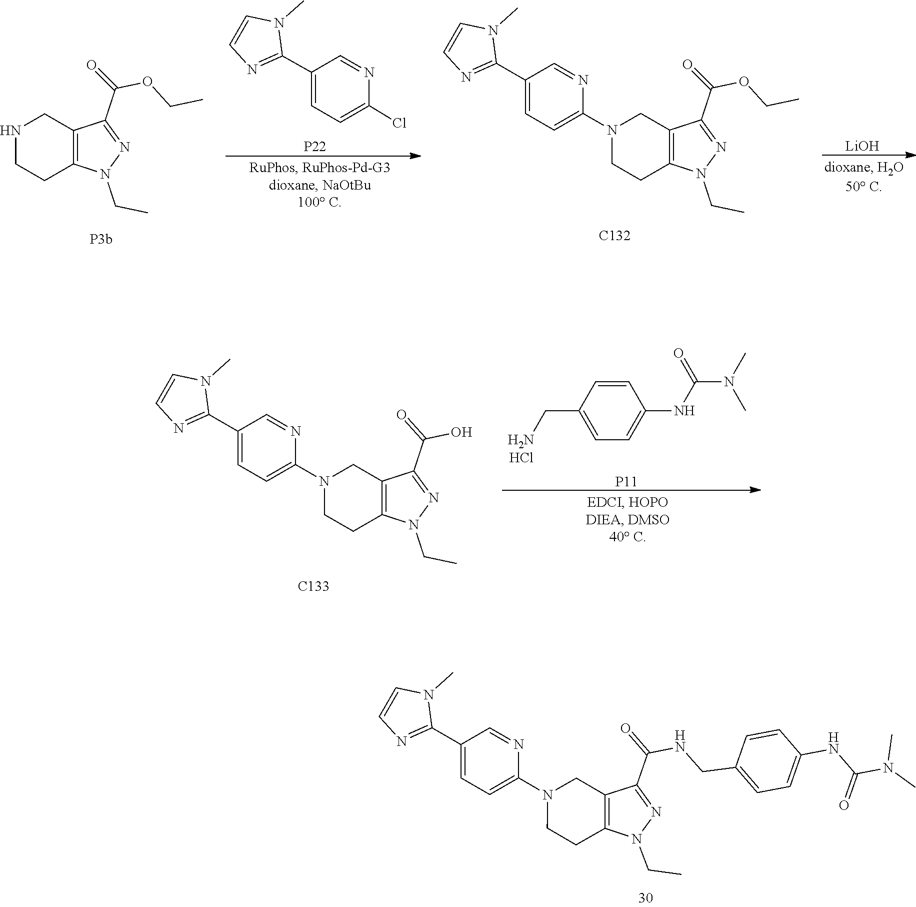

### Step 1. Preparation of ethyl 1-ethyl-5-(5-(1-methyl-1H-imidazol-2-yl)pyridin-2-yl)-4,5,6,7-tetrahydro-1H-pyrazolo[4,3-c]pyridine-3-carboxylate (C132)

To a solution of P3b (0.760 g, 2.93 mmol), P22 (0.680 g, 3.51 mmol) and NaOtBu (0.843 g, 8.78 mmol) in dioxane (15 mL) was added RuPhos (0.273 g, 0.585 mmol) and RuPhos Pd G3 (0.489 g, 0.585 mmol) at room temperature. The reaction mixture was degassed with nitrogen gas and then stirred at 100° C. for 4 h to provide C132 (1.11 g, crude) as a solution. The solution was used directly in the next step without further purification. (LC/MS) m/z (M-ethyl)⁺=353.2.

### Step 2. Preparation of 1-ethyl-5-(5-(1-methyl-1H-imidazol-2-yl)pyridin-2-yl)-4,5,6,7-tetrahydro-1H-pyrazolo[4,3-c]pyridine-3-carboxylic acid (C133)

To a solution of C132 (1.26 g, 3.31 mmol) in dioxane (15 mL) and H₂O (5 mL) was added LiOH (0.397 g, 16.6 mmol) at room temperature then stirred at 50° C. for 5 h. The reaction was purified by reverse phase HPLC (C₁₈ 150 mm×40 mm×5 μm column, Mobile Phase A: H₂O (0.05% formic acid)/Mobile Phase B: ACN, 0 to 35% of Mobile Phase B over 9 min, 100% of Mobile Phase B hold 2 min, flow rate=60 mL/min) and lyophilized to provide C133 (0.200 g, 17.1% yield) as a yellow solid. (LC/MS) m/z (M+H)⁺=353.1. ¹H NMR (400 MHz, CDCl₃) δ 8.43 (d, 1H), 8.22 (s, 1H), 8.02 (dd, 1H), 7.37 (d, 1H), 6.98 (d, 1H), 6.89 (d, 1H), 4.76 (s, 2H), 4.20-4.05 (m, 4H), 3.80 (s, 3H), 2.82 (t, 2H), 1.45 (t, 3H).

### Step 3. Preparation of N-(4-(3,3-dimethylureido)benzyl)-1-ethyl-5-(5-(1-methyl-1H-imidazol-2-yl)pyridin-2-yl)-4,5,6,7-tetrahydro-1H-pyrazolo[4,3-c]pyridine-3-carboxamide (30)

To a solution of C133 (0.080 g, 0.23 mmol) in DMSO (2 mL) was added EDCl (0.10 g, 0.54 mmol), HOPO (76 mg, 0.68 mmol) and DIEA (0.15 g, 1.1 mmol) and stirred for 30 min at 40° C. then P11 (78 mg, 0.34 mmol) was added. The reaction was stirred at 40° C. for 1 h. The residue was purified by reverse phase HPLC (C₁₈ 150 mm×30 mm×5 μm column, Mobile Phase A: H₂O (0.05% NH₄OH—NH₄HCO₃)/Mobile Phase B: ACN, 20 to 60% of Mobile Phase B over 15 min, 100% of Mobile Phase B hold 2 min, flow rate=30 mL/min) and lyophilized to provide 30 (44 mg, 37% yield) as a white solid. (LC/MS) m/z (M+H)⁺=528.4. ¹H NMR (400 MHz, CD₃OD) δ 8.37 (d, 1H), 7.80 (dd, 1H), 7.36-7.31 (m, 2H), 7.30-7.26 (m, 2H), 7.15 (d, 1H), 7.01 (d, 1H), 6.98 (d, 1H), 4.78 (s, 2H), 4.50 (s, 2H), 4.16-4.04 (m, 4H), 3.74 (s, 3H), 3.01 (s, 6H), 2.85 (t, 2H), 1.41 (t, 3H).

### Example 34

### N-(4-(3,3-Dimethylureido)benzyl)-1-isopropyl-5-(5-(1-methyl-1H-imidazol-2-yl)pyrimidin-2-yl)-4,5,6,7-tetrahydro-1H-pyrazolo[4,3-c]pyridine-3-carboxamide (34)

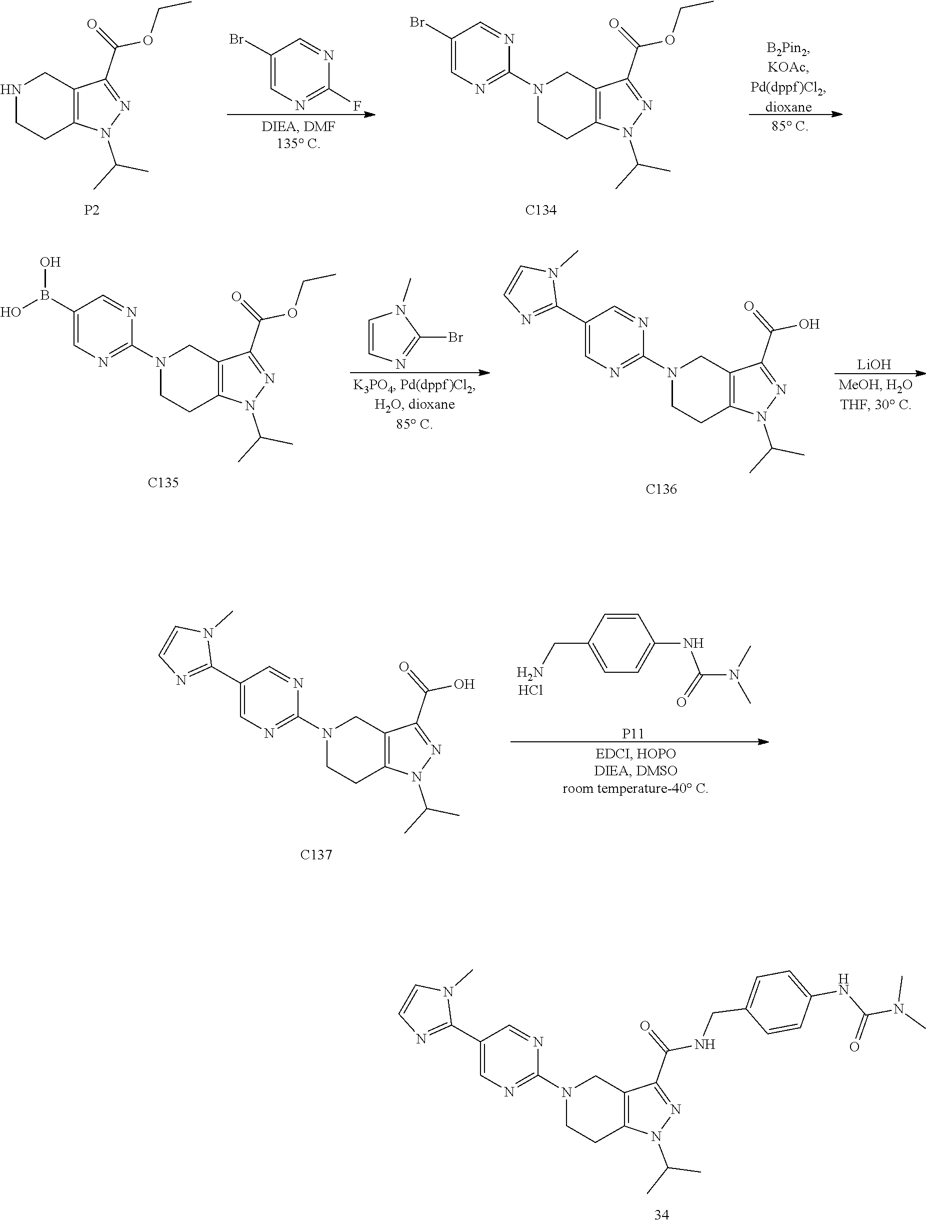

### Step 1. Preparation of ethyl 5-(5-bromopyrimidin-2-yl)-1-isopropyl-4,5,6,7-tetrahydro-1H-pyrazolo[4,3-c]pyridine-3-carboxylate (C134)

The reaction was conducted in two batches and then combined. To a solution of P2 (0.541 g, 1.98 mmol) and 5-bromo-2-fluoropyrimidine (CAS: 62802-38-4; 0.350 g, 1.98 mmol) in DMF (5 mL) was added DIEA (1.28 g, 9.89 mmol) and the reaction heated at 135° C. for 16 h to form the first batch. A second batch of the same reaction was conducted with P2 (77.4 mg, 0.283 mmol). The two batches were combined and diluted with EtOAc (50 mL). The suspension was washed with H₂O (3×30 mL) and then brine (20 mL). The organic layer was dried with Na₂SO₄ and then concentrated in vacuo. The residue was purified with column chromatography (silica gel, 0-23% EtOAc:PE) to provide C134 (0.750 g, 84.2% yield) as a white solid. (LC/MS) m/z (M+H)⁺=394.1. ¹H NMR (400 MHz, (CD₃)₂SO) δ 8.50 (s, 2H), 4.85 (s, 2H), 4.56-4.46 (m, 1H), 4.29 (q, 2H), 4.05 (t, 2H), 2.81 (t, 2H), 1.39-1.35 (m, 6H), 1.31 (t, 3H).

### Step 2. Preparation of (2-(3-(ethoxycarbonyl)-1-isopropyl-1,4,6,7-tetrahydro-5H-pyrazolo[4,3-c]pyridin-5-yl)pyrimidin-5-yl)boronic acid (C135)

To a reaction mixture of C134 (0.350 g, 0.888 mmol) and B₂Pin₂ (0.271 g, 1.07 mmol) in dioxane (5 mL) was added KOAc (0.261 g, 2.66 mmol) and Pd(dppf)Cl₂ (52.0 mg, 0.0710 mmol). The suspension was purged with nitrogen gas for 3 min and then stirred at 85° C. for 16 h to provide C135 (0.319 g, crude) as a reaction mixture, which was used directly in the next step without further purification. (LC/MS) m/z (M+H)⁺=360.0.

### Step 3. Preparation of ethyl 1-isopropyl-5-(5-(1-methyl-1H-imidazol-2-yl)pyrimidin-2-yl)-4,5,6,7-tetrahydro-1H-pyrazolo[4,3-c]pyridine-3-carboxylate (C136)

The same reaction was conducted in two batches and then combined. To a reaction mixture of C135 (0.319 g, 0.888 mmol) and 2-bromo-1-methyl-1H-imidazole (0.214 g, 1.33 mmol) in dioxane (2.00 mL) and H₂O (0.25 mL) was added Pd(dppf)Cl₂ (65.0 mg, 0.0888 mmol) and K₃PO₄ (0.566 g, 2.66 mmol) to form the first batch. The suspension of the first batch was purged with nitrogen gas then stirred at 85° C. for 2 h.

A second batch of the same reaction was conducted with C135 (46.0 mg, 0.128 mmol). The two batches were combined and diluted with EtOAc (30 mL). The suspension was washed with H₂O (2×20 mL) and then brine (20 mL). The organic layer was dried with Na₂SO₄ and then concentrated in vacuo. The residue was purified with column chromatography (silica gel, 0-50% THF:PE) to provide C136 (0.214 g, 53.1% yield) as a brown oil. (LC/MS) m/z (M+H)⁺=396.2.

### Step 4. Preparation of 1-isopropyl-5-(5-(1-methyl-1H-imidazol-2-yl)pyrimidin-2-yl)-4,5,6,7-tetrahydro-1H-pyrazolo[4,3-c]pyridine-3-carboxylic acid (C137)

To a solution of C136 (0.214 g, 0.541 mmol) in THF (1.00 mL), H₂O (0.25 mL), and MeOH (0.25 mL) was added LiOH (68.1 mg, 1.62 mmol). The reaction mixture was stirred at 30° C. for 2 h and then extracted with EtOAc (2×10 mL). The combined organic layer was washed with H₂O (5 mL). The aqueous layer was adjusted with citric acid to pH=3 and then extracted with DCM (3×5 mL). The combined organic layer was dried with Na₂SO₄ and then concentrated in vacuo. The residue was purified by reverse phase HPLC (C₁₈ 150 mm×40 mm×5 μm column, Mobile Phase A: H₂O (NH₄OH—NH₄HCO₃)/Mobile Phase B: ACN, 8 to 48% of Mobile Phase B over 9 min, 100% of Mobile Phase B hold 2 min, flow rate=60 mL/min) and lyophilized to provide C137 (90.0 mg, 45.3% yield) as a white solid. (LC/MS) m/z (M+H)⁺=368.2.

### Step 5. Preparation of N-(4-(3,3-dimethylureido)benzyl)-1-isopropyl-5-(5-(1-methyl-1H-imidazol-2-yl)pyrimidin-2-yl)-4,5,6,7-tetrahydro-1H-pyrazolo[4,3-c]pyridine-3-carboxamide (34)

The same reaction was conducted in two batches and then combined. To a solution of C137 (0.070 g, 0.19 mmol) in DMSO (1 mL) was added HOPO (63 mg, 0.56 mmol), EDCl (88 mg, 0.46 mmol), and DIEA (0.12 g, 0.95 mmol). The reaction mixture was stirred at room temperature for 5 min, and then P11 (66 mg, 0.29 mmol) was added to form the first batch. The suspension of the first batch was stirred at 40° C. for 2 h.

A second batch of the same reaction was conducted with C137 (0.020 g, 0.054 mmol). The two batches were combined then purified with reverse phase HPLC (Boston Prime C₁₈ 150 mm×30 mm×5 μm column, Mobile Phase A: H₂O (NH₄OH—NH₄HCO₃)/Mobile Phase B: ACN, 18 to 48% of Mobile Phase B over 9 min, 100% of Mobile Phase B hold 3 min, flow rate=25 mL/min) and lyophilized to provide 34 (68 mg, 51% yield) as a white solid. (LC/MS) m/z (M+H)+=543.5. ¹H NMR (400 MHz, (CD₃)₂SO) δ 8.69 (s, 2H), 8.34 (t, 1H), 8.23 (s, 1H), 7.41-7.36 (m, 2H), 7.24 (d, 1H), 7.19-7.14 (m, 2H), 6.95 (d, 1H), 4.96 (s, 2H), 4.52-4.43 (m, 1H), 4.34 (d, 2H), 4.14 (t, 2H), 3.71 (s, 3H), 2.90 (s, 6H), 2.83 (t, 2H), 1.39 (d, 6H).

### Example 35

### 5-(5-(Dimethylcarbamoyl)pyrimidin-2-yl)-N-(4-(3,3-dimethylureido)benzyl)-1-isopropyl-4,5,6,7-tetrahydro-1H-pyrazolo[4,3-c]pyridine-3-carboxamide (35)

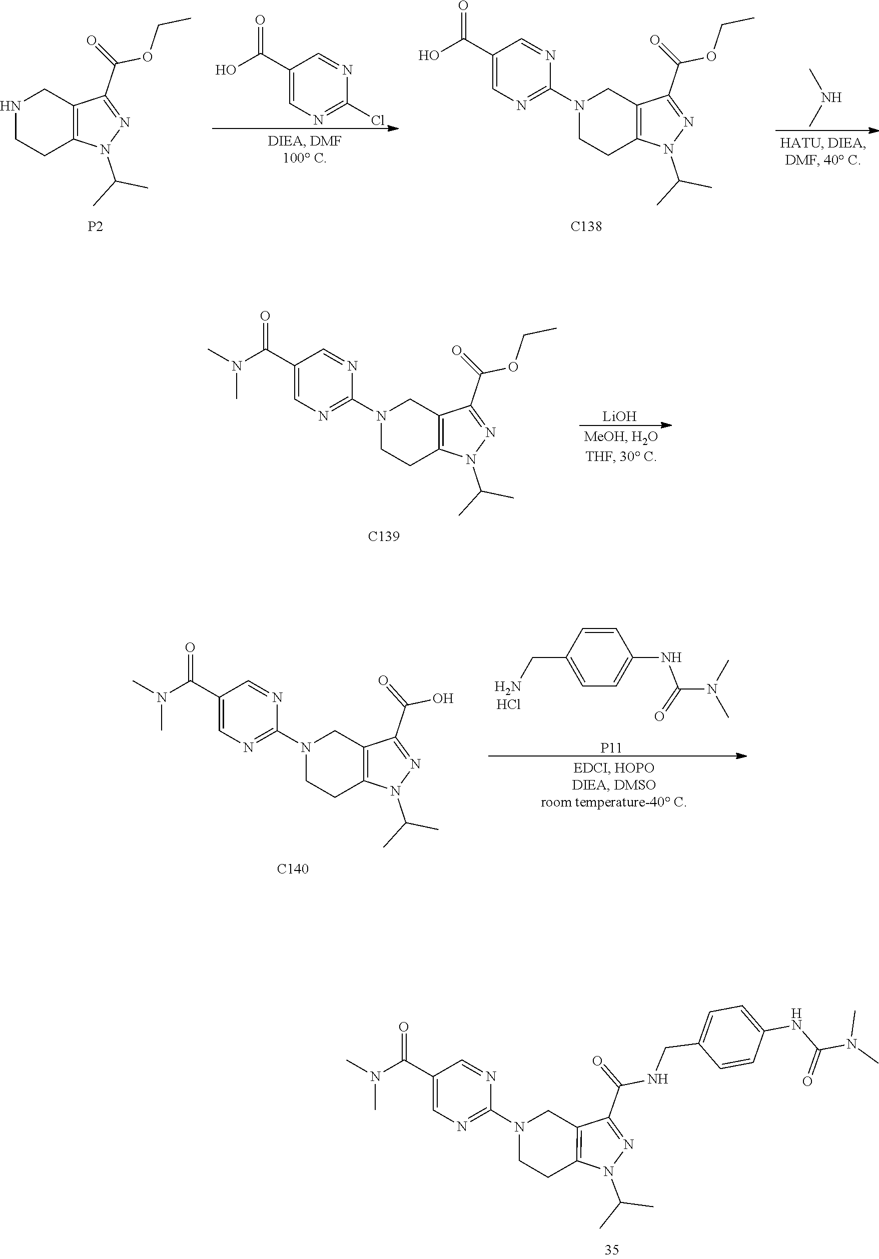

### Step 1. Preparation of 2-(3-(ethoxycarbonyl)-1-isopropyl-1,4,6,7-tetrahydro-5H-pyrazolo[4,3-c]pyridin-5-yl)pyrimidine-5-carboxylic acid (C138)

To a solution of P2 (0.350 g, 1.28 mmol) and 2-chloropyrimidine-5-carboxylic acid (CAS: 374068-01-6; 0.203 g, 1.28 mmol) in DMF (5 mL) was added DIEA (0.826 g, 6.39 mmol). The reaction mixture was stirred at 100° C. for 16 h to provide C138 (0.459 g, crude) as a suspension. The reaction mixture was used directly in the next step without further purification. (LC/MS) m/z (M+H)⁺=360.2.

### Step 2. Preparation of ethyl 5-(5-(dimethylcarbamoyl)pyrimidin-2-yl)-1-isopropyl-4,5,6,7-tetrahydro-1H-pyrazolo[4,3-c]pyridine-3-carboxylate (C139)

The same reaction was conducted in two batches and then combined. To a solution of C138 (0.459 g, 1.28 mmol) and dimethylamine hydrochloride (CAS: 506-59-2; 0.156 g, 1.91 mmol) in DMF (6 mL) was added DIEA (0.825 g, 6.39 mmol) and HATU (0.728 g, 1.92 mmol) at room temperature to form the first batch. The reaction mixture of the first batch was stirred at 40° C. for 1 h.

A second batch of the same reaction was conducted with C138 (39.0 mg, 0.108 mmol). The two batches were combined and diluted with EtOAc (50 mL). The suspension was washed with H₂O (3×30 mL) and then brine (30 mL). The organic layer was dried with Na₂SO₄ and then concentrated in vacuo. The residue was purified with column chromatography (silica gel, 0-54% THF:PE) to provide C139 (0.500 g, 89.4% yield) as an off-white solid. (LC/MS) m/z (M+H)⁺=387.3. ¹H NMR (400 MHz, (CD₃)₂SO) δ 8.54 (s, 2H), 4.95 (s, 2H), 4.59-4.48 (m, 1H), 4.30 (q, 2H), 4.15 (t, 2H), 2.99 (br s, 6H), 2.84 (t, 2H), 1.41-1.35 (m, 6H), 1.32 (t, 3H).

### Step 3. Preparation of 5-(5-(dimethylcarbamoyl)pyrimidin-2-yl)-1-isopropyl-4,5,6,7-tetrahydro-1H-pyrazolo[4,3-c]pyridine-3-carboxylic acid (C140)

The same reaction was conducted in two batches and then combined. To a solution of C139 (0.180 g, 0.466 mmol) in THF (1.00 mL), H₂O (0.25 mL), and MeOH (0.25 mL) was added LiOH (39.1 mg, 0.932 mmol) to form the first batch. The reaction mixture of the first batch was stirred at 30° C. for 2 h.

A second batch of the same reaction was conducted with C139 (20.0 mg, 0.0517 mmol). The two batches were combined and extracted with EtOAc (2×10 mL). The combined organic layer was washed with H₂O (10 mL). The aqueous layer was adjusted with citric acid to pH=3 and then extracted with DCM (5×50 mL). The combined organic layer was dried with Na₂SO₄ and then concentrated in vacuo to provide C140 (0.145 g, crude) as an off-yellow solid. The solid was used directly in the next step without further purification. (LC/MS) m/z (M+H)⁺=359.3.

### Step 4. Preparation of 5-(5-(dimethylcarbamoyl)pyrimidin-2-yl)-N-(4-(3,3-dimethylureido)benzyl)-1-isopropyl-4,5,6,7-tetrahydro-1H-pyrazolo[4,3-c]pyridine-3-carboxamide (35)

The same reaction was conducted in two batches and then combined. To a solution of C140 (0.060 g, 0.17 mmol) in DMSO (1 mL) was added HOPO (55 mg, 0.49 mmol), EDCl (77 mg, 0.40 mmol), and DIEA (0.11 g, 0.84 mmol). The reaction mixture was stirred at room temperature for 5 min, and then P11 (58 mg, 0.25 mmol) was added to form the first batch. The suspension of the first batch was stirred at 40° C. for 2 h.

A second batch of the same reaction was conducted with C140 (0.020 g, 0.056 mmol). The two batches were combined then purified with reverse phase HPLC (Boston Prime C₁₈ 150 mm×30 mm×5 μm column, Mobile Phase A: H₂O (NH₄OH—NH₄HCO₃)/Mobile Phase B: ACN, 15 to 45% of Mobile Phase B over 9 min, 100% of Mobile Phase B hold 3 min, flow rate=25 mL/min) and lyophilized to provide 35 (0.040 g, 38% yield) as a white solid. (LC/MS) m/z (M+H)⁺=534.5. ¹H NMR (400 MHz, (CD₃)₂SO) δ 8.51 (s, 2H), 8.34 (t, 1H), 8.23 (s, 1H), 7.41-7.35 (m, 2H), 7.16 (d, 2H), 4.94 (s, 2H), 4.53-4.42 (m, 1H), 4.33 (d, 2H), 4.12 (t, 2H), 2.97 (br s, 6H), 2.90 (s, 6H), 2.81 (t, 2H), 1.38 (d, 6H).

### Example 36

### N-(4-(3,3-Dimethylureido)benzyl)-1-ethyl-5-(5-(2-oxopyrrolidin-1-yl)pyridin-2-yl)-4,5,6,7-tetrahydro-1H-pyrazolo[4,3-c]pyridine-3-carboxamide (36)

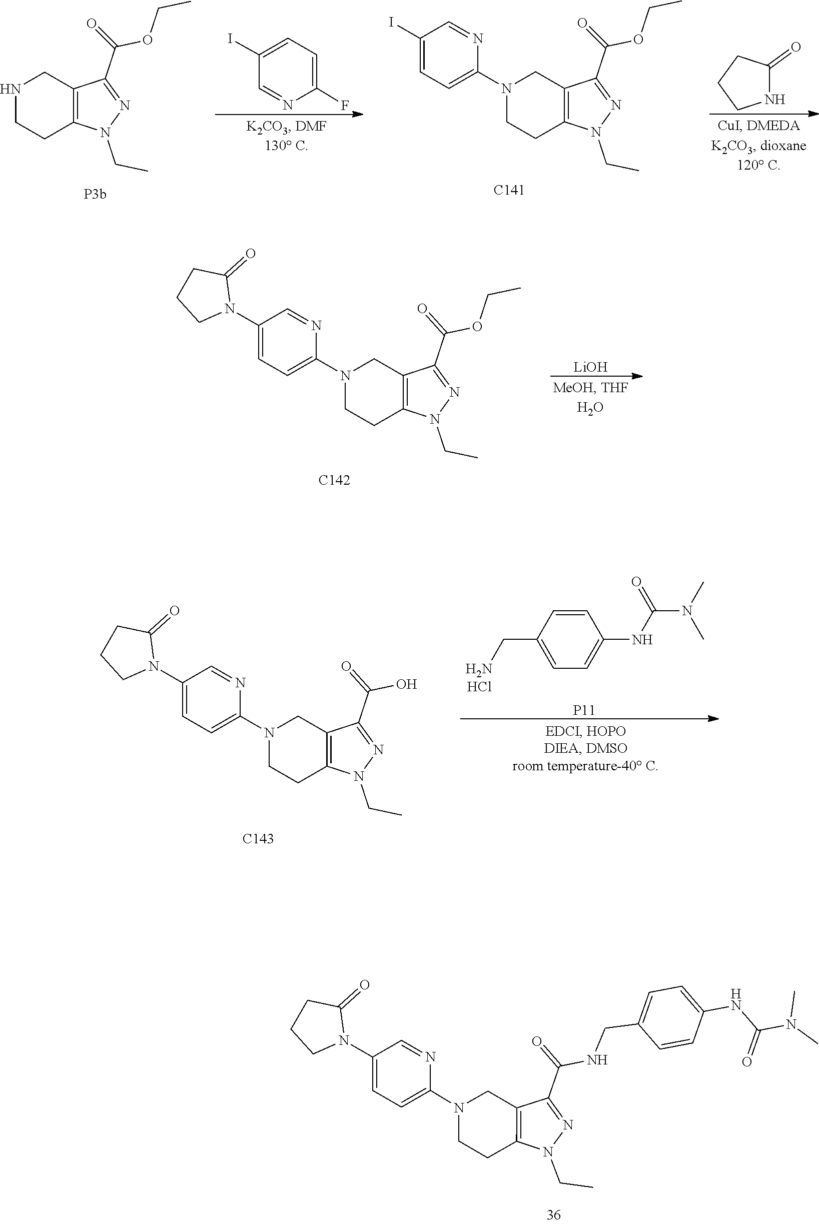

### Step 1. Preparation of ethyl 1-ethyl-5-(5-iodopyridin-2-yl)-4,5,6,7-tetrahydro-1H-pyrazolo[4,3-c]pyridine-3-carboxylate (C141)

A reaction mixture of P3b (0.803 g, 3.09 mmol), 2-fluoro-5-iodopyridine (CAS: 171197-80-1, 0.896 g, 4.02 mmol), and K₂CO₃ (1.28 g, 9.27 mmol) in DMF (20 mL) was stirred at 130° C. for 21 h. The brown reaction mixture was concentrated in vacuo and purified by column chromatography (silica gel, 0-40% EtOAc:PE) to provide C141 (0.632 g, 48.0% yield) as a white solid. (LC/MS) m/z (M+H)⁺=427.2. ¹H NMR (400 MHz, CDCl₃) δ 8.32 (d, 1H), 7.68 (dd, 1H), 6.61-6.55 (m, 1H), 4.61 (s, 2H), 4.43 (q, 2H), 4.18-4.11 (m, 2H), 4.03 (t, 2H), 2.78 (t, 2H), 1.47-1.38 (m, 6H).

### Step 2. Preparation of ethyl 1-ethyl-5-(5-(2-oxopyrrolidin-1-yl)pyridin-2-yl)-4,5,6,7-tetrahydro-1H-pyrazolo[4,3-c]pyridine-3-carboxylate (C142)

Under nitrogen gas, a reaction mixture of C141 (0.380 g, 0.891 mmol), pyrrolidine-2-one (0.114 g, 1.34 mmol), CuI (34.0 mg, 0.178 mmol), DMEDA (15.7 mg, 0.178 mmol) and K₂CO₃ (0.370 g, 2.68 mmol) in dioxane (10.0 mL) was heated at 120° C. for 16 h. The suspension was concentrated in vacuo and then purified by column chromatography (silica gel, 0-70% THF:PE) to provide C142 (0.337 g, 98.6% yield) as a white solid. (LC/MS) m/z (M+H)⁺=384.2.

### Step 3. Preparation of 1-ethyl-5-(5-(2-oxopyrrolidin-1-yl)pyridin-2-yl)-4,5,6,7-tetrahydro-1H-pyrazolo[4,3-c]pyridine-3-carboxylic acid (C143)

To a solution of C142 (0.300 g, 0.782 mmol) in THF (5.00 mL), MeOH (1.25 mL) and H₂O (2.50 mL) was added LiOH (49.7 mg, 2.07 mmol) then stirred at room temperature for 1 h. The solution was concentrated in vacuo to remove the organic solvents, then the resultant light-yellow aqueous solution was acidified to pH=3-4 with 2N HCl, which caused an off-white solid precipitate to form. The suspension was filtered, and then the filter cake was washed with H₂O (3×50 mL) and dried under high vacuo. The filter cake was collected and then lyophilized to provide C143 (0.170 g, 61.1% yield) as a white solid. ¹H NMR (400 MHz, CD₃OD) δ 8.29 (d, 1H), 7.82 (dd, 1H), 6.91 (d, 1H), 4.67 (s, 2H), 4.14 (q, 2H), 3.97 (t, 2H), 3.89-3.81 (m, 2H), 2.84 (t, 2H), 2.60-2.52 (m, 2H), 2.23-2.13 (m, 2H), 1.40 (t, 3H).

### Step 4. Preparation of N-(4-(3,3-dimethylureido)benzyl)-1-ethyl-5-(5-(2-oxopyrrolidin-1-yl)pyridin-2-yl)-4,5,6,7-tetrahydro-1H-pyrazolo[4,3-c]pyridine-3-carboxamide (36)

A solution of C143 (0.080 mg, 0.23 mmol), HOPO (74 mg, 0.66 mmol), EDCl (0.10 g, 0.54 mmol), and DIEA (0.14 g, 1.1 mmol) in DMSO (2.5 mL) was stirred at room temperature for 10 min, then P11 (62 mg, 0.27 mmol) was added and reaction mixture was stirred at 40° C. for 2 h. The residue was purified by reverse phase HPLC (C₁₈ 150 mm×30 mm×5 μm column, Mobile Phase A: H₂O/Mobile Phase B: ACN (NH₄OH—NH₄HCO₃), 15 to 55% of Mobile Phase B over 9 min, 100% of Mobile Phase B hold 2 min, flow rate=30 mL/min) and lyophilized to provide 36 (28 mg, 23% yield) as a white solid. (LC/MS) m/z (M+H)⁺=531.5. ¹H NMR (400 MHz, (CD₃)₂SO) δ 8.42 (t, 1H), 8.30 (d, 1H), 8.23 (s, 1H), 7.90 (dd, 1H), 7.43-7.36 (m, 2H), 7.21-7.15 (m, 2H), 6.92 (d, 1H), 4.67 (s, 2H), 4.34 (d, 2H), 4.08 (q, 2H), 3.88 (t, 2H), 3.78 (t, 2H), 2.92 (s, 6H), 2.83-2.77 (m, 2H), 2.44 (t, 2H), 2.11-2.01 (m, 2H), 1.33 (t, 3H).

### Example 37

### 5-(1-Acetyl-1,2,3,4-tetrahydroquinolin-6-yl)-N-(4-(3,3-dimethylureido)benzyl)-1-isopropyl-4,5,6,7-tetrahydro-1H-pyrazolo[4,3-c]pyridine-3-carboxamide (37)

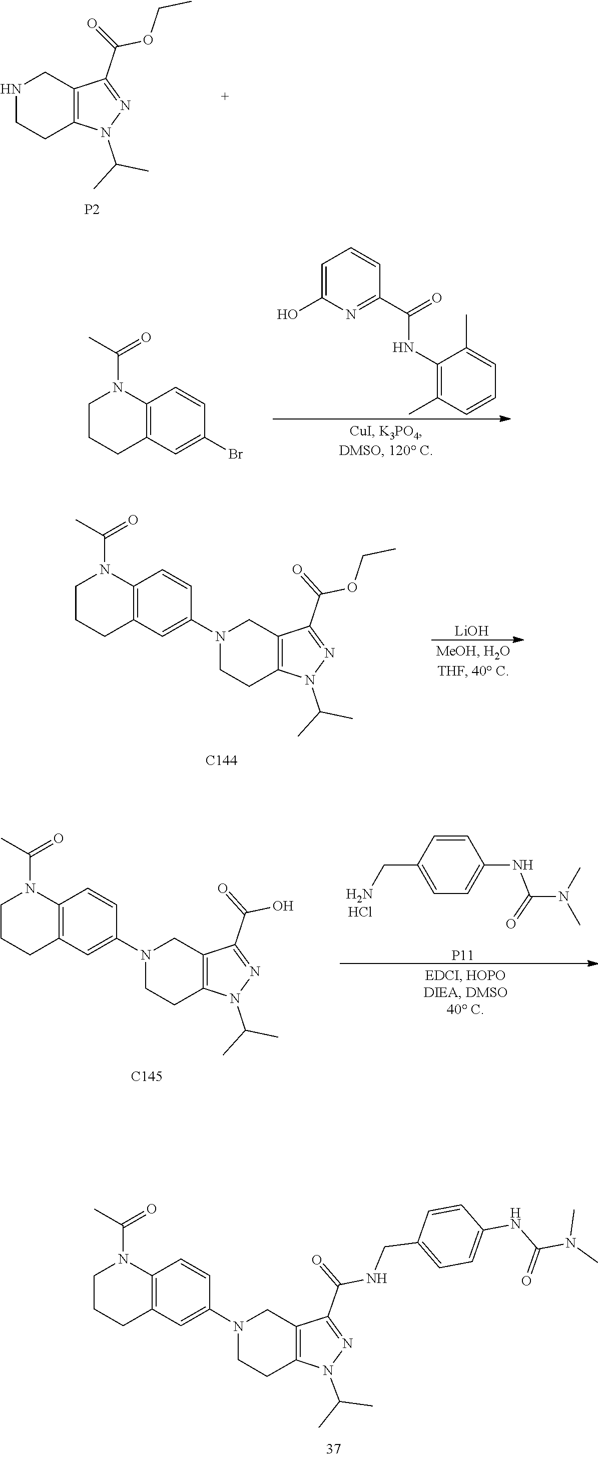

### Step 1. Preparation of ethyl 5-(1-acetyl-1,2,3,4-tetrahydroquinolin-6-yl)-1-isopropyl-4,5,6,7-tetrahydro-1H-pyrazolo[4,3-c]pyridine-3-carboxylate (C144)

The same reaction was conducted in two batches and then combined. To a solution of P2 (0.450 g, 1.90 mmol), N-(2,6-dimethylphenyl)-6-hydroxypyridine-2-carboxamide (CAS: 1982260-18-3; 0.122 g, 0.506 mmol) and 1-(6-bromo-3,4-dihydroquinolin-1(2H)-yl)ethan-1-one (CAS: 22190-40-5; 0.483 g, 1.90 mmol) in DMSO (20 mL) was added K₃PO₄ (1.07 g, 5.06 mmol) and CuI (48.2 mg, 0.253 mmol) at room temperature to form the first batch. The reaction mixture of the first batch was purged with nitrogen gas for 3 min and then stirred at 120° C. for 13 h.

A second batch of the same reaction was conducted with P2 (50.0 mg, 0.211 mmol). The two batches were combined and then concentrated in vacuo. The residue was purified with column chromatography (silica gel, 0-64% EtOAc:PE) to provide C144 (0.120 g, 15.4% yield) as a yellow solid. (LC/MS) m/z (M+H)⁺=411.1.

### Step 2. Preparation of 5-(1-acetyl-1,2,3,4-tetrahydroquinolin-6-yl)-1-isopropyl-4,5,6,7-tetrahydro-1H-pyrazolo[4,3-c]pyridine-3-carboxylic acid (C145)

To a solution C144 (0.120 g, 0.292 mmol) in THF (4 mL), MeOH (1 mL), and H₂O (2 mL), LiOH (24.5 mg, 0.585 mmol) was added at room temperature. The reaction mixture was stirred at 40° C. for 1 h and then was acidified with 2M HCl to pH=5. The acidic suspension was concentrated in vacuo and then lyophilized to provide an off-white solid. The solid was purified with reverse phase HPLC (C₁₈ 150 mm×30 mm×5 μm column, Mobile Phase A: H₂O (formic acid)/Mobile Phase B: ACN, 17 to 57% of Mobile Phase B over 15 min, 100% of Mobile Phase B hold 2 min, flow rate=30 mL/min) and lyophilized to provide C145 (70.0 mg, 62.5% yield) as a yellow solid. (LC/MS) m/z (M+H)⁺=383.3. ¹H NMR (400 MHz, CD₃OD) δ 7.15-7.04 (m, 1H), 6.93-6.83 (m, 2H), 4.58-4.49 (m, 1H), 4.37 (s, 2H), 3.74 (t, 2H), 3.62 (t, 2H), 2.87 (t, 2H), 2.75-2.64 (m, 2H), 2.27-2.11 (m, 3H), 2.00-1.87 (m, 2H), 1.48 (d, 6H).

### Step 3. Preparation of 5-(1-acetyl-1,2,3,4-tetrahydroquinolin-6-yl)-N-(4-(3,3-dimethylureido)benzyl)-1-isopropyl-4,5,6,7-tetrahydro-1H-pyrazolo[4,3-c]pyridine-3-carboxamide (37)

To a reaction mixture of C145 (0.070 g, 0.18 mmol), HOPO (61 mg, 0.55 mmol), and EDCl (84 mg, 0.44 mmol) in DMSO (2 mL) was added P11 (0.050 g, 0.22 mmol) and DIEA (0.14 g, 1.1 mmol). The suspension was stirred at room temperature for 10 min, and then the light-brown solution was stirred at 40° C. for 2 h. The light-brown reaction mixture was purified with reverse phase HPLC (C₁₈ 150 mm×30 mm×5 μm column, Mobile Phase A: H₂O (NH₄OH—NH₄HCO₃)/Mobile Phase B: ACN, 22 to 62% of Mobile Phase B over 9 min, 100% of Mobile Phase B hold 2 min) and lyophilized to provide 37 (0.055 g, 54% yield) as a white solid. (LC/MS) m/z (M+H)⁺=558.5. ¹H NMR (400 MHz, CD₃OD) δ 7.38-7.33 (m, 2H), 7.32-7.27 (m, 2H), 7.15-7.06 (m, 1H), 6.92 (d, 2H), 4.56-4.48 (m, 3H), 4.42 (s, 2H), 3.75 (t, 2H), 3.63 (t, 2H), 3.02 (s, 6H), 2.87 (t, 2H), 2.74-2.65 (m, 2H), 2.29-2.15 (m, 3H), 2.00-1.88 (m, 2H), 1.48 (d, 6H).

### Example 39

### N-(4-(3,3-Dimethylureido)benzyl)-5-(5-((4-(3,3-dimethylureido)benzyl)carbamoyl)pyridin-2-yl)-1-isopropyl-4,5,6,7-tetrahydro-1H-pyrazolo[4,3-c]pyridine-3-carboxamide (39)

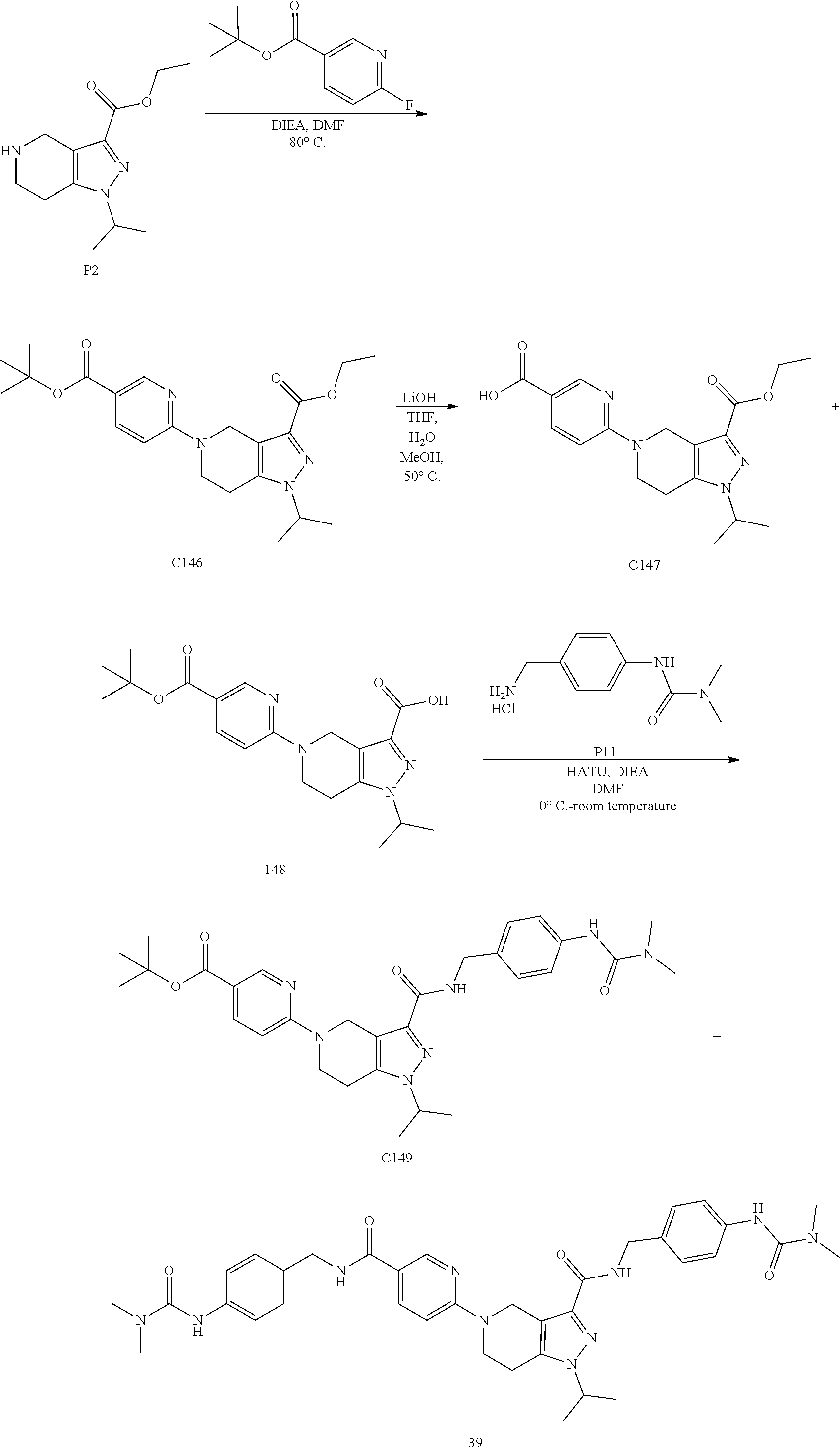

### Step 1: Preparation of ethyl 5-(5-(tert-butoxycarbonyl)pyridin-2-yl)-1-isopropyl-4,5,6,7-tetrahydro-1H-pyrazolo[4,3-c]pyridine-3-carboxylate (C146)

A reaction mixture of P2 (2.00 g, 7.30 mmol), tert-butyl 6-fluoronicotinate (CAS: 676560-01-3, 1.44 g, 7.30 mmol), and DIEA (2.83 g, 21.9 mmol) in DMF (60 mL) was stirred at 80° C. for 16 h. The reaction mixture was diluted with H₂O (200 mL) and then extracted with EtOAc (3×100 mL). The combined organic layer was washed with brine (2×100 mL) and then dried with Na₂SO₄ and filtered. The filtrate was concentrated in vacuo and then purified by column chromatography (silica gel, 0-25% EtOAc:PE) to provide C146 (1.80 g, 59.4% yield) as a white solid. ¹H NMR (400 MHz, (CD₃)₂SO) δ 8.64 (d, 1H), 7.94 (dd, 1H), 6.94 (d, 1H), 4.79 (s, 2H), 4.59-4.47 (m, 1H), 4.31 (q, 2H), 4.08-4.00 (m, 2H), 2.85 (t, 2H), 1.53 (s, 9H), 1.39 (d, 6H), 1.34 (t, 3H).

### Step 2. Preparation of 6-(3-(ethoxycarbonyl)-1-isopropyl-1,4,6,7-tetrahydro-5H-pyrazolo[4,3-c]pyridin-5-yl)nicotinic acid (C147) and 5-(5-(tert-butoxycarbonyl)pyridin-2-yl)-1-isopropyl-4,5,6,7-tetrahydro-1H-pyrazolo[4,3-c]pyridine-3-carboxylic acid (C148)

To a solution of C146 (1.80 mg, 4.34 mmol) in THF (10 mL), MeOH (5 mL) and H₂O (5 mL) was added LiOH (0.456 g, 10.9 mmol) at room temperature and then was stirred at 50° C. for 1 h. The LCMS of the reaction mixture showed 14.7% of C147 (LC/MS) m/z (M+H)⁺=331.1 and 84.6% of C148 (LC/MS) m/z (M+H)⁺=387.1 had formed. The reaction mixture was kept in the refrigerator for 48 h and then was concentrated in vacuo. The residue was diluted with H₂O (10 mL) and then acidified with 1N HCl to pH˜5, which caused a white solid precipitate. The suspension was filtered, and the filter cake was washed with H₂O (2×5 mL). The filter cake was collected then concentrated in vacuo to provide, according to LCMS, a mixture of 24% C147 and 76% C148 (1.60 g, crude) as a white solid. The solid was used directly in the next step without further purification.

C147: (LC/MS) m/z (M+H)⁺=331.2

C148 (LC/MS) m/z (M+H)⁺=387.1.

### Step 5. Preparation of N-(4-(3,3-dimethylureido)benzyl)-5-(5-((4-(3,3-dimethylureido)benzyl)carbamoyl)pyridin-2-yl)-1-isopropyl-4,5,6,7-tetrahydro-1H-pyrazolo[4,3-c]pyridine-3-carboxamide (39)

At 0° C., to a solution of C147 and C148 (0.500 g, 1.29 mmol) in DMF (10 mL) was added HATU (0.738 g, 1.94 mmol). The reaction mixture was stirred at 0° C. for 20 min then DIEA (0.669 g, 5.18 mmol) and P11 (0.446 g, 1.94 mmol) were added. After the addition, the reaction mixture was warmed to room temperature and stirred for 16 h. The LCMS of the reaction mixture showed 23.2% of 39 (LC/MS) m/z (M+H)⁺=681.4 and 43.8% of C149 (LC/MS) m/z (M+H)⁺=562.3 had formed. The suspension was diluted with H₂O (50 mL) and extracted with MeOH:DCM (1:10, 3×30 mL). The combined organic layer was washed with brine, dried with Na₂SO₄, and filtered. The filtrate was concentrated in vacuo then diluted with DMF (5 mL) and was purified by reverse phase HPLC (C₁₈ 150 mm×40 mm×5 μm column, Mobile Phase A: H₂O/Mobile Phase B: ACN (NH₄OH—NH₄HCO₃), 20 to 60% of Mobile Phase B over 9 min, 100% of Mobile Phase B hold 5 min, flow rate=30 mL/min) and lyophilized to provide 39 (108 mg, 12% yield) as a white solid. (LC/MS) m/z (M+H)⁺=681.5. ¹H NMR (400 MHz, (CD₃)₂SO) δ 8.75 (t, 1H), 8.65 (d, 1H), 8.34 (t, 1H), 8.23 (d, 2H), 8.01 (dd, 1H), 7.41-7.35 (m, 4H), 7.20-7.12 (m, 4H), 6.90 (d, 1H), 4.76 (s, 2H), 4.54-4.42 (m, 1H), 4.35 (dd, 4H), 3.97 (t, 2H), 2.81 (t, 2H), 1.39 (d, 6H).

### Example 41

### N-(4-(3,3-Dimethylureido)benzyl)-1-isopropyl-5-(5-(5-methyl-1H-pyrazol-1-yl)pyridin-2-yl)-4,5,6,7-tetrahydro-1H-pyrazolo[4,3-c]pyridine-3-carboxamide (41)

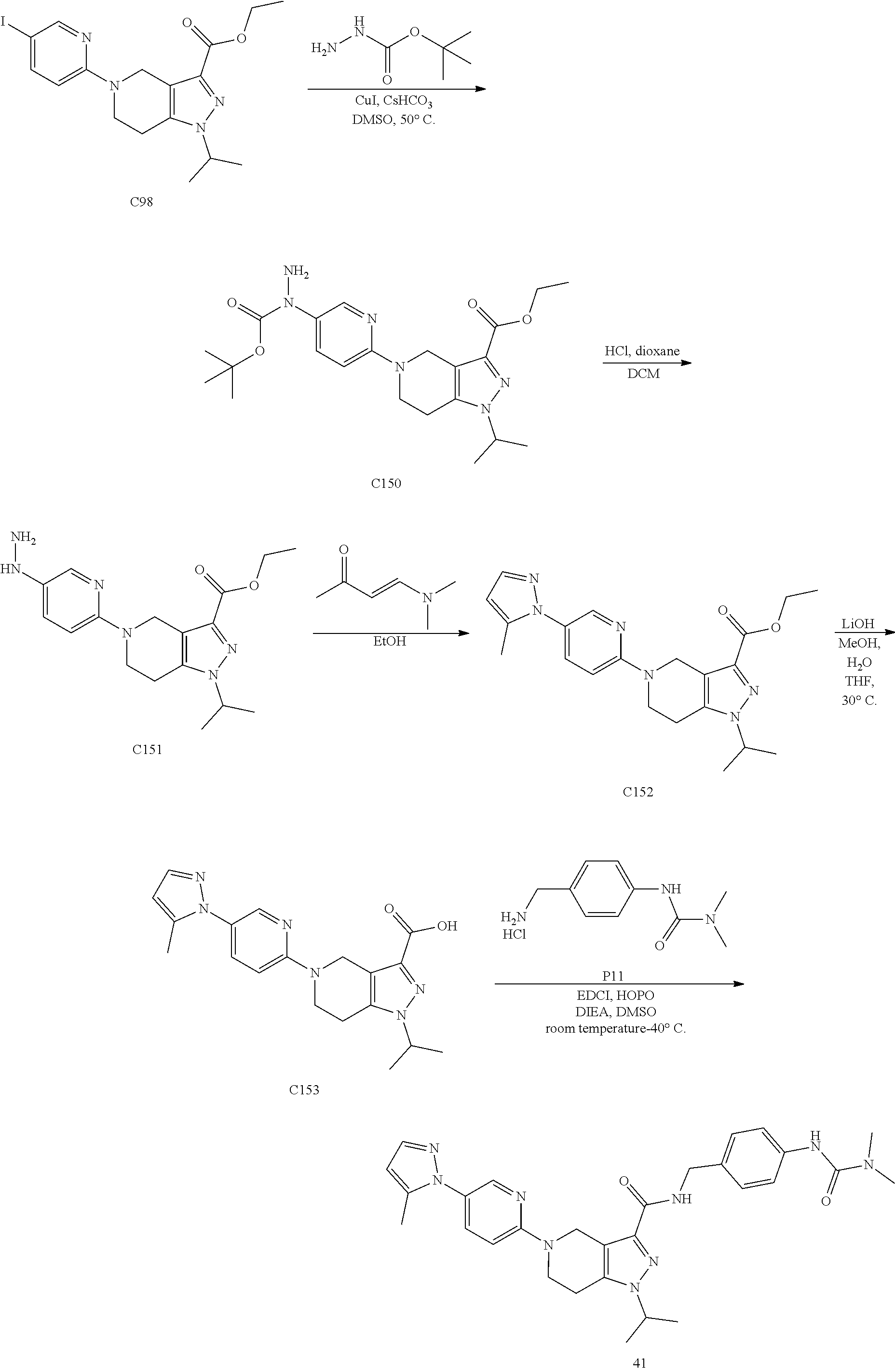

### Step 1. Preparation of ethyl 5-(5-(1-(tert-butoxycarbonyl)hydrazineyl)pyridin-2-yl)-1-isopropyl-4,5,6,7-tetrahydro-1H-pyrazolo[4,3-c]pyridine-3-carboxylate (C150)

Under nitrogen gas, to a reaction mixture of C98 (0.170 g, 0.386 mmol) in DMSO (1 mL) was added CuI (14.7 mg, 0.0772 mmol) and CsHCO₃ (0.377 g, 1.16 mmol) at room temperature. The suspension was stirred at 50° C. for 3 h and filtered through celite. The filtrate was concentrated in vacuo and then purified by column chromatography (silica gel, 0-54% THF:PE) to provide C150 (0.100 g, 58.3% yield) as a yellow oil. (LC/MS) m/z (M+H)⁺=445.2.

### Step 2. Preparation of ethyl 5-(5-hydrazineylpyridin-2-yl)-1-isopropyl-4,5,6,7-tetrahydro-1H-pyrazolo[4,3-c]pyridine-3-carboxylate hydrochloride (C151)

To a reaction mixture of C150 (0.10 g, 0.22 mmol) in DCM (1 mL) was added HCl in dioxane (8.2 mg, 0.22 mmol) and then stirred at room temperature for 16 h. The suspension was concentrated in vacuo to provide C151 (0.080 g, crude) as a solid. The solid was used directly in the next step without further purification. ¹H NMR (400 MHz, (CD₃)₂SO) δ 10.35 (br s, 2H), 7.90 (d, 1H), 7.76 (d, 1H), 7.44-7.25 (m, 1H), 6.87 (s, 1H), 4.70 (s, 2H), 4.59-4.49 (m, 1H), 4.31 (q, 2H), 3.96 (t, 2H), 2.95-2.87 (m, 2H), 1.40-1.36 (m, 6H), 1.35-1.30 (m, 3H).

### Step 3. Preparation of ethyl 1-isopropyl-5-(5-(5-methyl-1H-pyrazol-1-yl)pyridin-2-yl)-4,5,6,7-tetrahydro-1H-pyrazolo[4,3-c]pyridine-3-carboxylate (C152)

To a reaction mixture of C151 (0.080 g, 0.21 mmol) in EtOH (1 mL) was added 4-(dimethylamino)but-3-en-2-one (CAS: 1190-91-6, 24 mg, 0.21 mmol) and then stirred at room temperature for 16 h. The suspension was filtered through celite, and the filtrate was concentrated in vacuo. The residue was purified by column chromatography (silica gel, 0-30% EtOAc:PE) to provide C152 (28 mg, 31% yield) as a yellow oil. ¹H NMR (400 MHz, CDCl₃) δ 8.24 (d, 1H), 7.63-7.57 (m, 1H), 7.56 (d, 1H), 6.81 (d, 1H), 6.18 (s, 1H), 4.70 (s, 2H), 4.55-4.36 (m, 3H), 4.16-4.12 (m, 2H), 2.86 (t, 2H), 2.30 (s, 3H), 1.53 (d, 6H), 1.43 (t, 3H).

### Step 4. Preparation 1-isopropyl-5-(5-(5-methyl-1H-pyrazol-1-yl)pyridin-2-yl)-4,5,6,7-tetrahydro-1H-pyrazolo[4,3-c]pyridine-3-carboxylic acid (C153)

To a solution of C152 (25 mg, 0.063 mmol) in THF (1.00 mL), H₂O (0.25 mL), and MeOH (0.25 mL) was added LiOH (5.3 mg, 0.13 mmol). The reaction mixture was stirred at 30° C. for 2 h. The suspension's pH was adjusted with citric acid to pH=3 and concentrated in vacuo to provide C153 (23 mg, crude) as a yellow solid. The solid was used directly in the next step without further purification.

### Step 5. Preparation N-(4-(3,3-dimethylureido)benzyl)-1-isopropyl-5-(5-(5-methyl-1H-pyrazol-1-yl)pyridin-2-yl)-4,5,6,7-tetrahydro-1H-pyrazolo[4,3-c]pyridine-3-carboxamide (41)

To a solution of C153 (25 mg, 0.068 mmol) in DMSO (1 mL) was added HOPO (22 mg, 0.20 mmol), EDCl (31 mg, 0.16 mmol), and DIEA (44 mg, 0.34 mmol). The suspension was stirred at room temperature for 5 min, and then P11 (23 mg, 0.10 mmol) was added. The reaction mixture was stirred at 40° C. for 2 h then purified with reverse phase HPLC (Boston Prime C₁₈ 150 mm×30 mm×5 μm column, Mobile Phase A: H₂O (NH₄OH—NH₄HCO₃)/Mobile Phase B: ACN, 24 to 54% of Mobile Phase B over 9 min, 100% of Mobile Phase B hold 3 min, flow rate=50 mL/min) and lyophilized to provide 41 (26 mg, 71% yield) as a white solid. (LC/MS) m/z (M+H)⁺=542.4. ¹H NMR (400 MHz, CDCl₃) δ 8.22 (d, 1H), 7.58-7.53 (m, 2H), 7.38-7.33 (m, 2H), 7.30 (d, 2H), 7.15 (t, 1H), 6.86 (d, 1H), 6.31 (s, 1H), 6.17 (s, 1H), 4.76 (s, 2H), 4.56 (d, 2H), 4.36-4.31 (m, 1H), 4.15 (t, 2H), 3.03 (s, 6H), 2.82 (t, 2H), 2.29 (s, 3H), 1.45 (d, 6H).

### Example 42

### N-(4-(3,3-Dimethylureido)benzyl)-7-(5-(1-methyl-1H-imidazol-2-yl)pyridin-2-yl)-3-(trifluoromethyl)-5,6,7,8-tetrahydroimidazo[1,5-a]pyrazine-1-carboxamide (42)

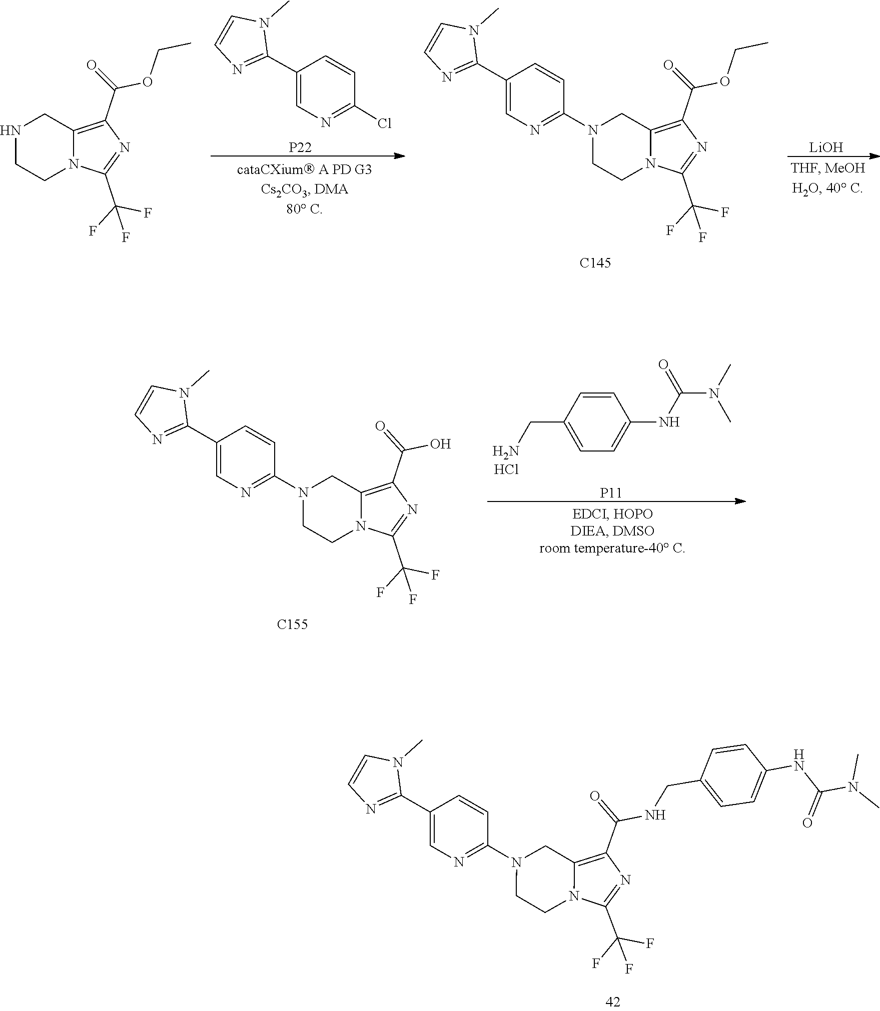

### Step 1. Preparation of ethyl 7-(5-(1-methyl-1H-imidazol-2-yl)pyridin-2-yl)-3-(trifluoromethyl)-5,6,7,8-tetrahydroimidazo[1,5-a]pyrazine-1-carboxylate (C154)

To the reaction mixture of ethyl 5,6,7,8-tetrahydro-3-(trifluoromethyl)imidazo[1,5-a]pyrazine-1-carboxylate (CAS: 1598419-92-1; 0.300 g, 1.14 mmol) and P22 (0.243 g, 1.25 mmol) in DMA (10 mL) was added cataCXium® A Pd G3 (83.0 mg, 0.114 mmol) and Cs₂CO₃ (1.11 g, 3.42 mmol). The suspension was degassed with nitrogen gas and then stirred at 80° C. for 16 h. The reaction mixture was diluted with DCM (3×50 mL). The combined organic layer was washed with H₂O (50 mL), then brine (50 mL), and dried with Na₂SO₄. The suspension was concentrated in vacuo and then purified by column chromatography (silica gel, 4% MeOH:EtOAc) to provide C154 (0.258 g, 53.8% yield) as a yellow gum. (LC/MS) m/z (M+H)⁺=421.2.

### Step 2. Preparation of 7-(5-(1-methyl-1H-imidazol-2-yl)pyridin-2-yl)-3-(trifluoromethyl)-5,6,7,8-tetrahydroimidazo[1,5-a]pyrazine-1-carboxylic acid (C155)

To C154 (0.258 g, 0.614 mmol) in THF (4.0 mL), H₂O (2.0 mL), and MeOH (1.0 mL) was added LiOH (44.1 mg, 1.84 mmol) at room temperature. The reaction mixture was stirred at 40° C. for 1.5 h and then was concentrated in vacuo to remove the organic layer. The resultant aqueous layer was acidified to pH=5-6 with 2N HCl, which caused an off-white solid precipitate. The suspension was filtered, and then the filter cake was washed with H₂O (3×10 mL) and dried under high vacuo. The filter cake was collected then lyophilized to provide C155 (0.120 g, 49.8% yield) as a yellow solid. ¹H NMR (400 MHz, (CD₃)₂SO) δ 12.48 (br s, 1H), 8.51 (d, 1H), 7.95 (dd, 1H), 7.24 (s, 1H), 7.10 (d, 1H), 6.97 (s, 1H), 5.12 (s, 2H), 4.31 (t, 2H), 4.14 (t, 2H), 3.73 (s, 3H)

### Step 3. Preparation of N-(4-(3,3-dimethylureido)benzyl)-7-(5-(1-methyl-1H-imidazol-2-yl)pyridin-2-yl)-3-(trifluoromethyl)-5,6,7,8-tetrahydroimidazo[1,5-a]pyrazine-1-carboxamide (42)

To a solution of C155 (0.050 g, 0.13 mmol) in DMSO (2.0 mL) was added HOPO (42 mg, 0.38 mmol), DIEA (82 mg, 0.64 mmol), and EDCl (59 mg, 0.31 mmol). The reaction mixture was stirred for 10 min at room temperature, and then P11 (38 mg, 0.17 mmol) was added. The reaction mixture was stirred at 40° C. for 3 h then purified by reverse phase HPLC (Phenomenex Gemini NX C₁₈ 150 mm×30 mm×5 μm column, Mobile Phase A: H₂O (NH₄OH—NH₄HCO₃)/Mobile Phase B: ACN, 22 to 62% of Mobile Phase B over 9 min, 100% of Mobile Phase B hold 2 min, flow rate=60 mL/min) and lyophilized to provide 42 (0.030 mg, 42% yield) as a white solid. (LC/MS) m/z (M+H)⁺=568.3. ¹H NMR (400 MHz, CD₃OD) δ 8.43 (d, 1H), 7.86 (dd, 1H), 7.35-7.30 (m, 2H), 7.29-7.24 (m, 2H), 7.16 (d, 1H), 7.07-7.00 (m, 2H), 5.15 (s, 2H), 4.49 (s, 2H), 4.34 (t, 2H), 4.21 (t, 2H), 3.74 (s, 3H), 3.00 (s, 6H).

### Example 43

### 5-(5-(Dimethylcarbamoyl)pyridin-2-yl)-1-isopropyl-N-(4-(6-oxo-1,6-dihydropyrimidin-2-yl)benzyl)-4,5,6,7-tetrahydro-1H-pyrazolo[4,3-c]pyridine-3-carboxamide (43)

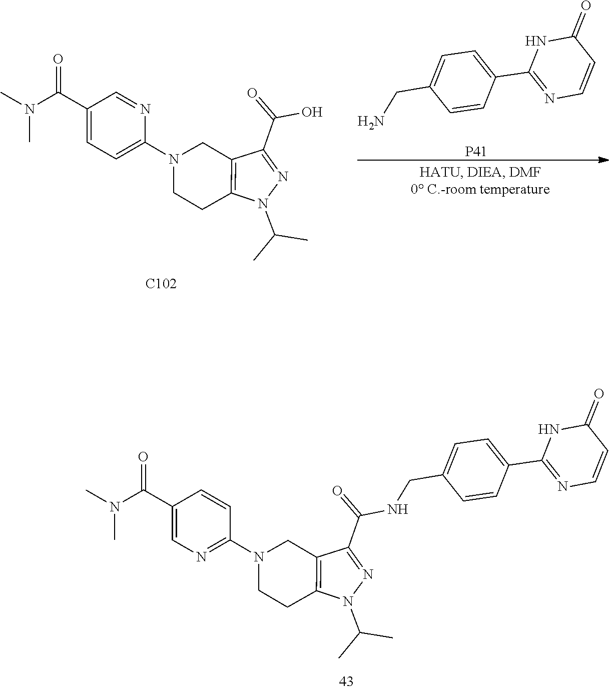 [SMILES: CC(C)n1nc(C(=O)O)c2c1CCN(c1ccc(C(=O)N(C)C)cn1)C2 ; "P41" ; NCc1ccc(-c2nccc(=O)[nH]2)cc1 ; "C102" ; CC(C)n1nc(C(=O)NCc2ccc(-c3nccc(=O)[nH]3)cc2)c2c1CCN(c1ccc(C(=O)N(C)C)cn1)C2]

### Step 1. Preparation of 5-(5-(dimethylcarbamoyl)pyridin-2-yl)-1-isopropyl-N-(4-(6-oxo-1,6-dihydropyrimidin-2-yl)benzyl)-4,5,6,7-tetrahydro-1H-pyrazolo[4,3-c]pyridine-3-carboxamide (43)

Under nitrogen gas at 0° C., to a solution of C102 (91 mg, 0.38 mmol), P41 (0.14 g, 0.38 mmol) and HATU (0.15 g, 0.40 mmol) in DMF (1.9 mL) was added DIEA (0.22 g, 1.7 mmol). The reaction mixture was stirred at room temperature for 2 h then purified by reverse phase HPLC (Boston Prime C₁₈ 150 mm×30 mm×5 μm column, Mobile Phase A: H₂O (0.05% formic acid)/Mobile Phase B: ACN, 15 to 35% of Mobile Phase B over 11.0 min, 95% of Mobile Phase B hold 2.0 min, flow rate=35 mL/min) to provide 43 (0.010 g, 4.8% yield) as a white solid. (LC/MS) m/z (M+H)⁺=541.3. ¹H NMR (400 MHz, (CD₃)₂SO) δ 8.57 (t, 1H), 8.23 (d, 1H), 8.09-8.02 (m, 3H), 7.63 (dd, 1H), 7.43 (d, 2H), 6.88 (d, 1H), 6.29 (d, 1H), 4.74 (s, 2H), 4.57-4.41 (m, 3H), 3.95 (t, 2H), 2.96 (s, 6H), 2.82 (t, 2H), 1.40 (d, 6H).

### Example 45

### N-(4-(3,3-Dimethylureido)benzyl)-1-isopropyl-5-(5-(1-methyl-1H-imidazol-2-yl)pyridin-2-yl)-4,5,6,7-tetrahydro-1H-pyrazolo[4,3-c]pyridine-3-carboxamide (45)

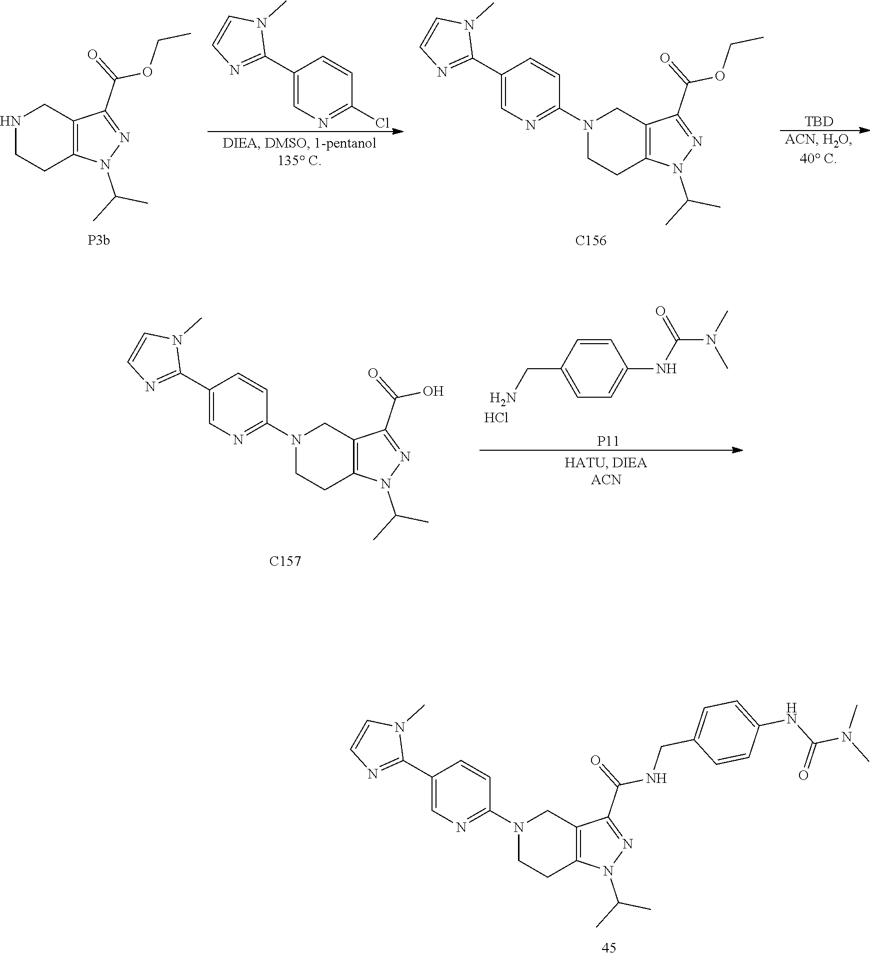

### Step 1. Preparation of ethyl 1-isopropyl-5-(5-(1-methyl-1H-imidazol-2-yl)pyridin-2-yl)-4,5,6,7-tetrahydro-1H-pyrazolo[4,3-c]pyridine-3-carboxylate (C156)

A reaction mixture of P3b (0.100 g, 0.421 mmol), P22 (97.9 mg, 0.506 mmol), and DIEA (0.218 g, 1.69 mmol) in DMSO (1 mL) and 1-pentanol (1 mL) was heated at 135° C. for 17 h. The solution was diluted with DCM and brine and then concentrated in vacuo. The residue was redissolved in DCM and then purified by column chromatography (silica gel, 0-10% MeOH:DCM) to provide C156 (0.130 g, 78.3% yield) as a yellow oil. (LC/MS) m/z (M+H)⁺=395.3.

### Step 2. Preparation of 1-isopropyl-5-(5-(1-methyl-1H-imidazol-2-yl)pyridin-2-yl)-4,5,6,7-tetrahydro-1H-pyrazolo[4,3-c]pyridine-3-carboxylic acid (C157)

To a solution of C156 (0.050 g, 0.13 mmol) in ACN (1 mL) and H₂O (46 μL) was added TBD (53 mg, 0.38 mmol). The reaction mixture was stirred at 40° C. for 1 h and 20 min, and then NaOH (0.020 g, 0.51 mmol) was added. The suspension was stirred at room temperature for 15 min, and then 12.1M HCl (18 mg, 0.51 mmol) was added. The reaction mixture was concentrated in vacuo to provide C157 (0.046 g, crude) as a yellow oil. The oil was used directly in the next step without further purification.

### Step 3. Preparation of N-(4-(3,3-dimethylureido)benzyl)-1-isopropyl-5-(5-(1-methyl-1H-imidazol-2-yl)pyridin-2-yl)-4,5,6,7-tetrahydro-1H-pyrazolo[4,3-c]pyridine-3-carboxamide (45)

A reaction mixture of C157 (46 mg, 0.13 mmol), P11 (35 mg, 0.15 mmol), DIEA (49 mg, 0.38 mmol), and HATU (48 mg, 0.13 mmol) in ACN (1 mL) was stirred at room temperature for 21 h and then concentrated in vacuo. The residue was dissolved in DMSO then acidified with TFA and purified by reverse phase HPLC (Sunfire C₁₈ 100 mm×19 mm×5 μm column, Mobile Phase A: H₂O/Mobile Phase B: ACN (0.05% TFA), 5 to 95% of Mobile Phase B over 9 min, 95% of Mobile Phase B hold 1 min, flow rate=25 mL/min) and lyophilized to provide 45 (4.3 mg, 6.3% yield). (LC/MS) m/z (M+H)⁺=542.3. ¹H NMR (600 MHz, (CD₃)₂SO) δ 8.44 (d, 1H), 8.34 (t, 1H), 8.23 (s, 1H), 7.86 (dd, 1H), 7.42-7.37 (m, 2H), 7.24 (br s, 1H), 7.20-7.16 (m, 2H), 6.98 (d, 2H), 4.77 (s, 2H), 4.55-4.47 (m, 1H), 4.35 (d, 2H), 3.97 (t, 2H), 3.72 (s, 3H) 2.91 (s, 6H), 2.85 (t, 2H), 1.41 (d, 6H).

### Example 77

### rac-(R)-5-(5-(Dimethylcarbamoyl)pyridin-2-yl)-N-(4-(3,3-dimethylureido)benzyl)-7-ethyl-1-methyl-4,5,6,7-tetrahydro-1H-pyrazolo[4,3-c]pyridine-3-carboxamide (77)

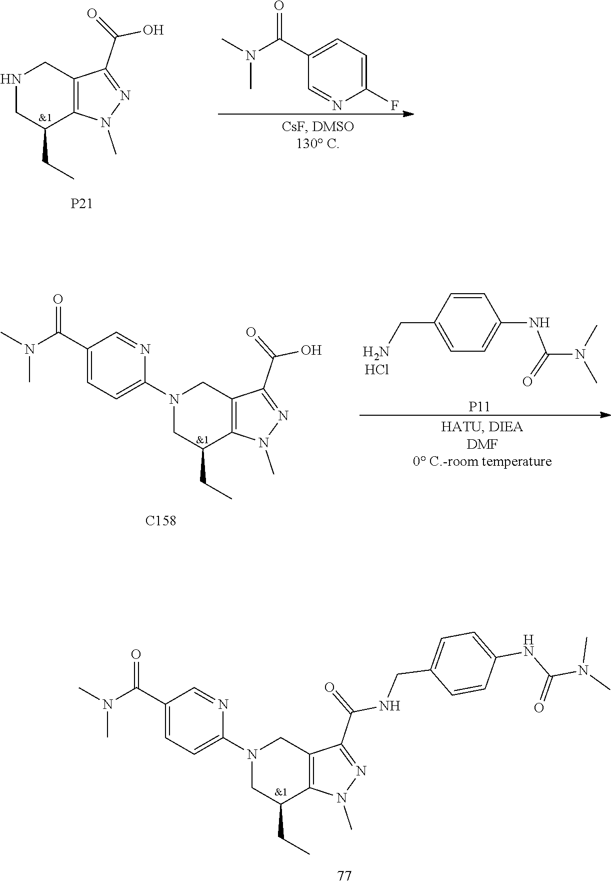

### Step 1. Preparation of rac-(R)-5-(5-(dimethylcarbamoyl)pyridin-2-yl)-7-ethyl-1-methyl-4,5,6,7-tetrahydro-1H-pyrazolo[4,3-c]pyridine-3-carboxylic acid (C158)

To a reaction mixture of P21 (0.186 g, 0.757 mmol) and CsF (0.287 g, 1.89 mmol) in DMSO (3 mL) was added 6-fluoro-N,N-dimethylnicotinamide (CAS: 1032251-82-3; 0.127 g, 0.757 mmol). The suspension was stirred at 130° C. for 8 h and then was diluted with H₂O (20 mL). The reaction mixture was cooled to 0° C., and then aqueous Na₂CO₃ was added to pH=11 and extracted with EtOAc (2×25 mL). The aqueous layer was collected and adjusted to pH=3 with 1M HCl. The acidic aqueous phase was extracted with (1:10, MeOH:DCM, 3×20 mL). The combined organic layer was washed with brine (2×25 mL), dried over Na₂SO₄, filtered, and concentrated in vacuo to provide C158 (0.271 g, crude) as a yellow oil. The oil was used directly in the next step without further purification. ¹H NMR (400 MHz, (CD₃)₂SO) δ 12.67 (s, 1H), 8.22 (d, 1H), 7.61 (dd, 1H), 6.82 (d, 1H), 4.97 (d, 1H), 4.64 (d, 1H), 4.16 (d, 1H), 3.83-3.74 (m, 3H), 3.19-3.12 (m, 1H), 3.00-2.90 (m, 7H), 1.62-1.51 (m, 1H), 1.46-1.33 (m, 1H), 0.99 (t, 3H).

### Step 2. Preparation of rac-(R)-5-(5-(dimethylcarbamoyl)pyridin-2-yl)-N-(4-(3,3-dimethylureido)benzyl)-7-ethyl-1-methyl-4,5,6,7-tetrahydro-1H-pyrazolo[4,3-c]pyridine-3-carboxamide (77)

At 0° C., to a solution of C158 (0.27 g, 0.68 mmol) in DMF (5 mL) was added HATU (0.28 g, 0.75 mmol). The reaction mixture was stirred at 0° C. for 20 min, and then DIEA (0.35 g, 2.7 mmol) and P11 (0.16 g, 0.68 mmol) were added at 0° C. The suspension was stirred at room temperature for 2 h then diluted with H₂O (50 mL) and extracted with (1:10, MeOH:DCM, 3×40 mL). The combined organic layer was dried with Na₂SO₄, filtered, and concentrated in vacuo to give a yellow gum. The residue was purified by column chromatography (silica gel; 0-10% MeOH:DCM) to provide 77 (0.32 g, 88% yield) as a yellow solid. (LC/MS) m/z (M+H)⁺=533.4. ¹H NMR (400 MHz, (CD₃)₂SO) δ 8.51 (t, 1H), 8.27-8.20 (m, 2H), 7.65 (dd, 1H), 7.41-7.35 (m, 2H), 7.18-7.12 (m, 2H), 6.83 (d, 1H), 5.01 (d, 1H), 4.73 (d, 1H), 4.40-4.18 (m, 3H), 3.82 (s, 3H), 3.23-3.09 (m, 1H), 3.04-2.94 (m, 7H), 2.93-2.87 (m, 6H), 1.66-1.53 (m, 1H), 1.49-1.36 (m, 1H), 1.03 (t, 3H).

### Example 58 and 78

### rel-(R or S)-5-(5-(Dimethylcarbamoyl)pyridin-2-yl)-N-(4-(3,3-dimethylureido)benzyl)-7-ethyl-1-methyl-4,5,6,7-tetrahydro-1H-pyrazolo[4,3-c]pyridine-3-carboxamide (58 and 78)

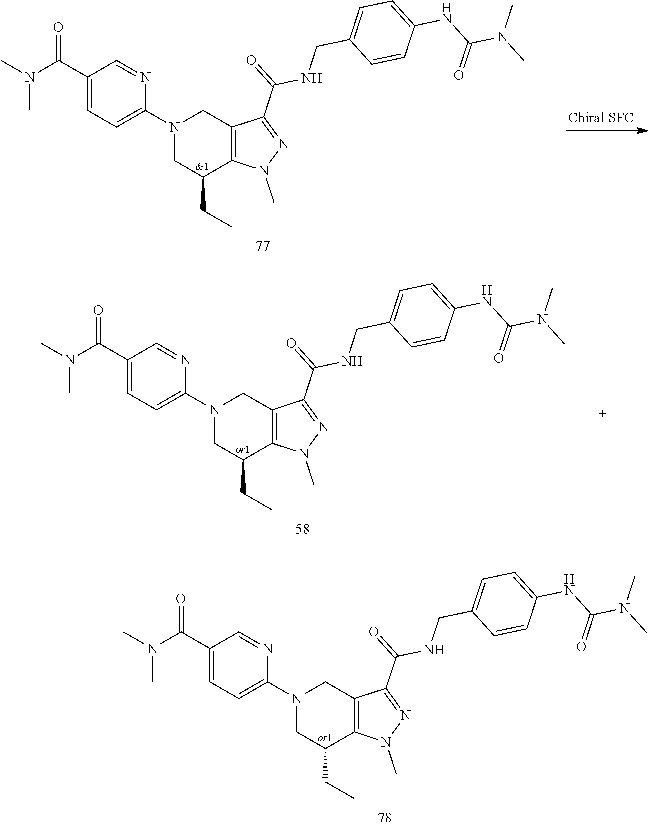

### Step 1. Preparation of rel-(R or S)-5-(5-(dimethylcarbamoyl)pyridin-2-yl)-N-(4-(3,3-dimethylureido)benzyl)-7-ethyl-1-methyl-4,5,6,7-tetrahydro-1H-pyrazolo[4,3-c]pyridine-3-carboxamide (58 and 78)

Example 77 (0.29 g, 0.81 mmol) was separated using chiral SFC [Column: DAICEL CHIRALCEL OD 250 mm×30 mm×10 μm; Mobile phase: 40% CO₂/60% MeOH (0.1% NH₄OH); flow rate: 80 mL/min; gradient time=45 min] to provide 58 (87 mg, 30% yield) as a white solid and 78 (83 mg, 29% yield) as a white solid. The chiral purity was determined using SFC analytical method on Chiralcel OD-3 50 mm×4.6 mm×3 μm column; Mobile phase A: CO₂; Mobile phase B: MeOH with 0.2% of NH₃; isocratic: 40% of Mobile Phase B; flow rate: 3.5 mL/min; backpressure: 1500 psi; column temperature: 35° C. to provide Example 58: peak 1 ((SFC-MS) m/z (M+H)⁺=533.41 at retention time: 0.778 min, 100% ee). ¹H NMR (400 MHz, (CD₃)₂SO) δ 8.52 (t, 1H), 8.27-8.21 (m, 2H), 7.65 (dd, 1H), 7.40-7.34 (m, 2H), 7.18-7.11 (m, 2H), 6.83 (d, 1H), 5.00 (d, 1H), 4.76-4.69 (m, 1H), 4.42-4.16 (m, 3H), 3.81 (s, 3H), 3.24-3.15 (m, 1H), 3.02-2.93 (m, 7H), 2.92-2.87 (m, 6H), 1.66-1.53 (m, 1H), 1.50-1.36 (m, 1H), 1.02 (t, 3H) and Example 78: peak 2 ((SFC-MS) m/z (M+H)⁺=533.41 at retention time: 1.392 min, 100% ee). ¹H NMR (400 MHz, (CD₃)₂SO) δ 8.49 (t, 1H), 8.24-8.16 (m, 2H), 7.61 (dd, 1H), 7.36-7.31 (m, 2H), 7.16-7.09 (m, 2H), 6.80 (d, 1H), 4.97 (d, 1H), 4.69 (d, 1H), 4.35-4.15 (m, 3H), 3.78 (s, 3H), 3.20-3.10 (m, 1H), 2.98-2.91 (m, 7H), 2.87 (m, 6H), 1.62-1.49 (m, 1H), 1.46-1.32 (m, 1H), 0.99 (t, 3H).

### Example 102

### N-(4-Aminobenzyl)-5-(5-(dimethylcarbamoyl)pyridin-2-yl)-1-isopropyl-4,5,6,7-tetrahydro-1H-pyrazolo[4,3-c]pyridine-3-carboxamide (102)

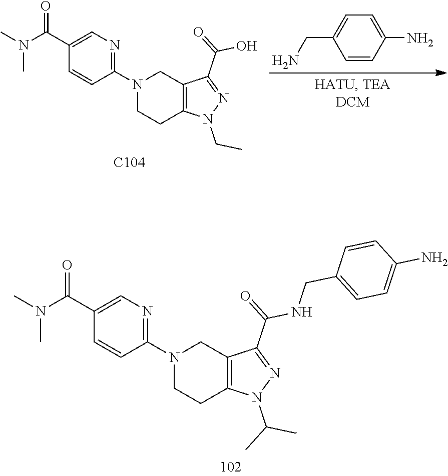 [SMILES: CCn1nc(C(=O)O)c2c1CCN(c1ccc(C(=O)N(C)C)cn1)C2 ; NCc1ccc(N)cc1 ; "C104" ; CC(C)n1nc(C(=O)NCc2ccc(N)cc2)c2c1CCN(c1ccc(C(=O)N(C)C)cn1)C2]

### Step 1. Preparation of N-(4-aminobenzyl)-5-(5-(dimethylcarbamoyl)pyridin-2-yl)-1-isopropyl-4,5,6,7-tetrahydro-1H-pyrazolo[4,3-c]pyridine-3-carboxamide (102)

To a reaction mixture of C104 (1.00 g, 2.80 mmol) in DCM (15 mL) were added 4-aminobenzylamine (CAS: 4403-71-8; 0.431 g, 3.53 mmol), HATU (2.13 g, 5.60 mmol), and TEA (0.581 g, 5.74 mmol). The suspension was stirred at room temperature for 2 h, then diluted with DCM and washed with H₂O. The organic layer was dried with Na₂SO₄, filtered, and concentrated in vacuo. The residue was purified by column chromatography (silica gel, 0-10% MeOH:DCM) to provide 102 (1.58 g, >95.0% yield) as a yellow foam. (LCMS) m/z (M+H)⁺=462.6. ¹H NMR (500 MHz, (CD₃)₂SO) δ 8.24 (d, 1H), 8.11 (t, 1H), 7.64 (dd, 1H), 6.99 (d, 2H), 6.88 (d, 1H), 6.50 (d, 2H), 4.94 (s, 2H), 4.74 (s, 2H), 4.54-4.43 (m, 1H), 4.24 (d, 2H), 3.95 (t, 2H), 2.97 (s, 6H), 2.81 (t, 2H), 1.38 (d, 6H).

### Example 113

### (R)-5-(5-(Dimethylcarbamoyl)pyridin-2-yl)-N-(4-(3,3-dimethylureido)benzyl)-1-ethyl-6-methyl-4,5,6,7-tetrahydro-1H-pyrazolo[4,3-c]pyridine-3-carboxamide (113)

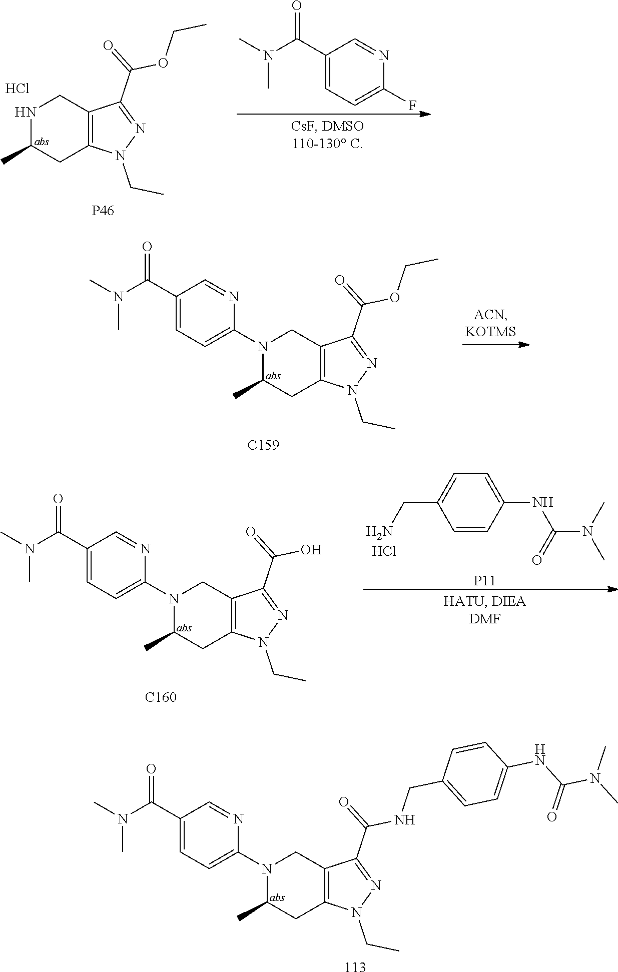

### Step 1. Preparation of ethyl (R)-5-(5-(dimethylcarbamoyl)pyridin-2-yl)-1-ethyl-6-methyl-4,5,6,7-tetrahydro-1H-pyrazolo[4,3-c]pyridine-3-carboxylate (C159)

To a solution of P46 (0.380 g, 1.39 mmol) and 6-fluoro-N,N-dimethylnicotinamide (CAS: 1032251-82-3; 233 mg, 1.39 mmol) in DMSO (5 mL) was added CsF (527 mg, 3.47 mmol). The reaction mixture was heated at 130° C. for 8 h, and then an additional portion of 6-fluoro-N,N-dimethylnicotinamide (CAS: 1032251-82-3; 233 mg, 1.39 mmol) was added. The suspension was heated at 110° C. for 24 h, and then an additional portion of CsF (274 mg, 1.80 mmol) was added. The reaction mixture was stirred at 130° C. for 16 h and then was diluted with H₂O (10 mL). The suspension was extracted with EtOAc (3×10 mL). The combined organic layer was washed with brine (2×10 mL), dried over Na₂SO₄, filtered, and concentrated in vacuo. The residue was purified by column chromatography (silica gel, 0-100% EtOAc:PE) to provide C159 (0.250 g, 46.7% yield) as a yellow gum. (LC/MS) m/z (M+H)⁺=386.2. ¹H NMR (600 MHz, (CD₃)₂SO) δ 8.30-8.27 (m, 1H), 7.68 (dd, 1H), 6.89 (d, 1H), 5.33-5.28 (m, 1H), 5.14 (d, 1H), 4.31 (q, 2H), 4.18-4.09 (m, 3H), 3.04-2.93 (m, 7H), 2.77 (d, 1H), 1.36-1.30 (m, 6H), 1.08 (d, 3H).

### Step 2. Preparation of (R)-5-(5-(dimethylcarbamoyl)pyridin-2-yl)-1-ethyl-6-methyl-4,5,6,7-tetrahydro-1H-pyrazolo[4,3-c]pyridine-3-carboxylic acid (C160)

To a reaction mixture of C159 (0.250 g, 0.649 mmol) in ACN (5 mL) was added KOTMS (166 mg, 1.30 mmol) at room temperature under nitrogen gas and then stirred for 1 h. The solution was acidified with HCl in dioxane (47.3 mg, 1.30 mmol) to a pH of 5-6, then concentrated in vacuo to provide C160 (232 mg, crude) as a yellow gum. The gum was used directly in the next step without further purification. (LC/MS) m/z (M+H)⁺=358.1. ¹H NMR (600 MHz, (CD₃)₂SO) δ 8.26 (d, 1H), 7.71 (d, 1H), 6.95 (d, 1H), 5.25-5.20 (m, 1H), 5.12 (d, 1H), 4.17-4.06 (m, 3H), 2.98 (s, 7H), 2.77 (d, 1H), 1.32 (t, 3H), 1.08 (d, 3H).

### Step 3. Preparation of (R)-5-(5-(dimethylcarbamoyl)pyridin-2-yl)-N-(4-(3,3-dimethylureido)benzyl)-1-ethyl-6-methyl-4,5,6,7-tetrahydro-1H-pyrazolo[4,3-c]pyridine-3-carboxamide (113)

To a reaction mixture of C160 (232 mg, 0.649 mmol) and P11 (125 mg, 0.649 mmol) in DMF (2.5 mL) were added HATU (271 mg, 0.714 mmol) and DIEA (336 mg, 2.60 mmol). The suspension was stirred at room temperature for 16 h, diluted with H₂O (15 mL), and extracted with EtOAc (3×15 mL). The combined organic layer was washed with H₂O (2×10 mL), 1M HCl (2×10 mL), 1M Na₂CO₃ (2×10 mL), and then brine (1×). The organic layer was dried with Na₂SO₄, filtered, and concentrated in vacuo to provide a yellow solid. The solid was purified by reverse phase HPLC (Boston Prime C₁₈ 150 mm×30 mm×5 μm column, Mobile Phase A: H₂O (NH₄OH—NH₄HCO₃)/Mobile Phase B: ACN, 18 to 58% of Mobile Phase B over 10.0 min, 100% of Mobile Phase B hold 2.5 min, flow rate=30 mL/min) then lyophilized to provide 113 (134 mg, 38.8% yield) as a white solid. (LC/MS) m/z (M+H)⁺=533.3. ¹H NMR (400 MHz, (CD₃)₂SO) δ 8.47 (t, 1H), 8.26 (d, 1H), 8.23 (s, 1H), 7.65 (dd, 1H), 7.41-7.34 (m, 2H), 7.21-7.15 (m, 2H), 6.85 (d, 1H), 5.31-5.22 (m, 1H), 5.14 (d, 1H), 4.33 (d, 2H), 4.17-4.01 (m, 3H), 3.01-2.93 (m, 7H), 2.90 (s, 6H), 2.77-2.69 (m, 1H), 1.32 (t, 3H), 1.06 (d, 3H). The chiral purity was determined using SFC analytical method on Chiralpak AD-3 50 mm×4.6 mm×3 μm column; Mobile phase A: CO₂/Mobile phase B: EtOH with 0.2% of NH₃; isocratic gradient: 40% of Mobile Phase B; backpressure: 1500 psi; flow rate: 3.5 mL/min; column temperature: 35° C. to provide 113: peak 1 ((SFC-MS) m/z (M+H)⁺=533.43 at retention time: 1.503 min, 100% ee).

### Example 125

### (S)-5-(5-(Dimethylcarbamoyl)pyrimidin-2-yl)-N-(4-(3,3-dimethylureido)benzyl)-1-ethyl-7-methyl-4,5,6,7-tetrahydro-1H-pyrazolo[4,3-c]pyridine-3-carboxamide (125)

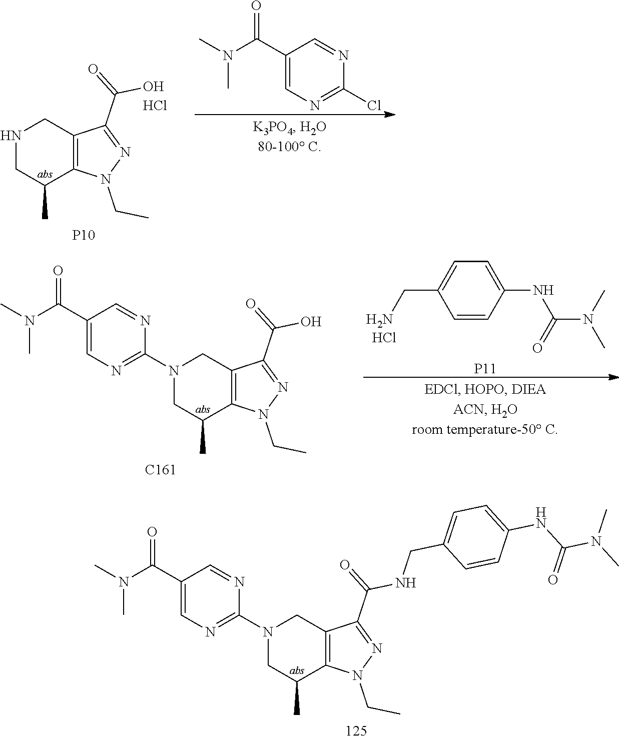

### Step 1. Preparation of (S)-5-(5-(dimethylcarbamoyl)pyrimidin-2-yl)-1-ethyl-7-methyl-4,5,6,7-tetrahydro-1H-pyrazolo[4,3-c]pyridine-3-carboxylic acid hydrochloride (C161)

To a solution of P10 (6.60 g, 26.9 mmol) and 2-chloro-N,N-dimethylpyrimidine-5-carboxamide (CAS: 1637587-62-2; 4.98 g, 26.9 mmol) in H₂O (130 mL) was added K₃PO₄ (17.1 g, 80.6 mmol) at room temperature. The reaction mixture was stirred between 80-100° C. for 18 h and then cooled to room temperature. The suspension was extracted MeOH:DCM (100 mL), and then the organic layer was discarded. The aqueous layer was acidified to pH<5 with concentrated HCl and then extracted with (1:10) MeOH:DCM (150 mL×3). The combined organic layer was dried over Na₂SO₄ and concentrated in vacuo to provide C161 (8.20 g, crude) as a yellow solid. The solid was used directly in the next step without further purification. (LCMS) m/z (M+H)⁺=359.2. ¹H NMR (400 MHz, (CD₃)₂SO) δ 12.74 (s, 1H), 8.53 (s, 2H), 5.38 (d, 1H), 4.69 (dd, 1H), 4.34 (d, 1H), 4.18-4.03 (m, 2H), 3.46 (dd, 1H), 3.28-3.19 (m, 1H), 2.99 (br s, 6H), 1.37 (t, 3H), 1.15 (d, 3H). The chiral purity was determined using Chiral HPLC analytical method on Chiralcel OJ-RH 150 mm×4.6 mm×5 μm column; Mobile phase A: H₂O (1.5 mL TFA)/Mobile phase B: ACN (1.5 mL TFA); 10-80% of Mobile phase B in 8 min then from 80-10% of Mobile phase B in 1 min and held at 10% of Mobile phase B for 6 min; flow rate: 0.8 mL/min; column temperature: 35° C. to provide C161: peak 1 (retention time: 7.659 min, 100% ee).

### Step 2. Preparation of (S)-5-(5-(dimethylcarbamoyl)pyrimidin-2-yl)-N-(4-(3,3-dimethylureido)benzyl)-1-ethyl-7-methyl-4,5,6,7-tetrahydro-1H-pyrazolo[4,3-c]pyridine-3-carboxamid (125)

To a solution of C161 (2.70 g, 6.84 mmol), EDCl (1.64 g, 8.55 mmol), and HOPO (0.950 g, 8.55 mmol) in ACN (35 mL) was added DIEA (2.21 g, 17.1 mmol). The solution was stirred at 50° C. for 3.5 h, which caused precipitate to form. The reaction mixture was cooled to room temperature and stirred for 15 min. Then, P11 (1.65 g, 7.18 mmol), H₂O (0.123 g, 6.84 mmol), and an additional portion of DIEA (3.53 g, 27.3 mmol) were added. The reaction mixture was stirred at 50° C. for 2 h, diluted with EtOAc (50 mL), and washed with H₂O (2×50 mL). The aqueous layer was re-extracted with EtOAc (50 mL). The combined organic layer was washed with saturated NaHCO₃ (2×50 mL) and brine (50 mL). The organic layer was dried over Na₂SO₄, filtered, and concentrated in vacuo to give a yellow solid. The yellow solid was purified by column chromatography (silica gel; 0-5% MeOH:DCM) then lyophilized to give 125 (3.39 g, 92.9% yield) as a white solid. (LCMS) m/z (M+H)⁺=534.3. ¹H NMR (400 MHz, (CD₃)₂SO) δ 8.52 (s, 2H), 8.45 (t, 1H), 8.23 (s, 1H), 7.41-7.35 (m, 2H), 7.19-7.13 (m, 2H), 5.41 (d, 1H), 4.67 (dd, 1H), 4.42-4.26 (m, 3H), 4.16-4.04 (m, 2H), 3.48 (dd, 1H), 3.28-3.19 (m, 1H), 2.99 (br s, 6H), 2.90 (s, 6H), 1.38 (t, 3H), 1.16 (d, 3H). The chiral purity was determined using SFC analytical method on Chiralcel OD-3 50 mm×4.6 mm×3 μm column; Mobile phase A: CO₂/Mobile phase B: EtOH with 0.2% of NH₃; gradient: 5-40% of Mobile Phase B in 1.5 min then held at 40% of Mobile phase B for 1.0 min then held at 5% of Mobile phase B for 0.5 min; backpressure: 1500 psi; flow rate: 4.0 mL/min; column temperature: 35° C. to provide 125: peak 1 ((SFC-MS) m/z (M+H)⁺=534.52 at retention time: 1.866 min, 100% ee). [α]²⁰_{D}=−154.134 (c=8.0 (g/L) in MeOH.

### Example 126

### (S)-5-(5-(Dimethylcarbamoyl)pyridin-2-yl)-N-(4-(3,3-dimethylureido)benzyl)-1-ethyl-7-methyl-4,5,6,7-tetrahydro-1H-pyrazolo[4,3-c]pyridine-3-carboxamide (126)

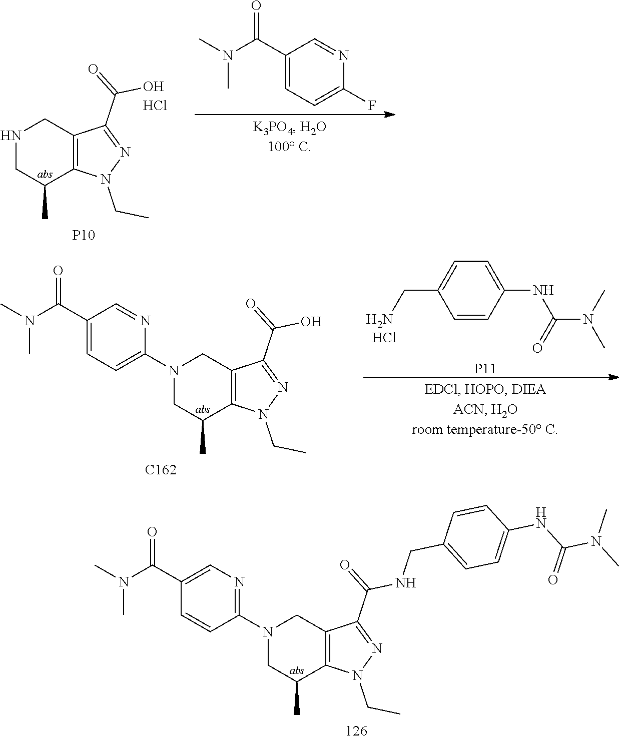

### Step 1. Preparation of (S)-5-(5-(dimethylcarbamoyl)pyridin-2-yl)-1-ethyl-7-methyl-4,5,6,7-tetrahydro-1H-pyrazolo[4,3-c]pyridine-3-carboxylic acid (C162)

To a solution of 6-fluoro-N,N-dimethylnicotinamide (CAS: 1032251-82-3; 3.61 g, 21.5 mmol) and P10 (5.56 g, 22.6 mmol) in H₂O (110 mL) was added K₃PO₄ (14.4 g, 67.9 mmol) at room temperature. The reaction mixture was stirred at 100° C. for 48 h and then cooled to room temperature. The suspension was extracted with MTBE (20 mL), and then the organic layer was discarded. The aqueous phase was acidified to pH˜5-6 with concentrated HCl and then extracted with (1:10) MeOH:DCM (40 mL×3). The combined organic layer was dried over Na₂SO₄ and concentrated in vacuo to provide C162 (6.35 g, crude) as a yellow solid. The solid was used directly in the next step without further purification. (LCMS) m/z (M+H)⁺=358.0. ¹H NMR (400 MHz, CDCl₃) δ 9.28 (br s, 1H), 8.43 (d, 1H), 7.71 (dd, 1H), 6.77 (d, 1H), 5.01 (d, 1H), 4.46-4.34 (m, 2H), 4.22-4.04 (m, 2H), 3.49-3.40 (m, 1H), 3.15-3.04 (m, 7H), 1.47 (t, 3H), 1.29 (d, 3H). The chiral purity was determined using SFC analytical method on Chiralcel OD-3 50 mm×4.6 mm×3 μm column; Mobile phase A: CO₂/Mobile phase B: EtOH with 0.2% of NH₃; gradient: 5-40% of Mobile Phase B in 1.5 min then held at 40% of Mobile phase B for 1.0 min then held at 5% of Mobile phase B for 0.5 min; backpressure: 1500 psi; flow rate: 4.0 mL/min; column temperature: 35° C. to provide C162: peak 1 ((SFC-MS) m/z (M+H)⁺=358.15 at retention time: 1.497 min, 100% ee).

### Step 2. Preparation of (S)-5-(5-(dimethylcarbamoyl)pyridin-2-yl)-N-(4-(3,3-dimethylureido)benzyl)-1-ethyl-7-methyl-4,5,6,7-tetrahydro-1H-pyrazolo[4,3-c]pyridine-3-carboxamide (126)

To a solution of C162 (3.00 g, 8.39 mmol), EDCl (2.01 g, 10.5 mmol), and HOPO (1.17 g, 10.5 mmol) in ACN (42 mL) was added DIEA (2.71 g, 21.0 mmol). The solution was stirred at 50° C. for 2 h, which caused precipitate to form. The reaction mixture was cooled to room temperature and stirred for 15 min. Then, P11 (2.02 g, 8.81 mmol), H₂O (0.151 g, 8.39 mmol), and an additional portion of DIEA (4.34 g, 33.6 mmol) were added. The reaction mixture was stirred at 50° C. for 4 h, diluted with EtOAc (30 mL), and washed with H₂O (2×30 mL). The aqueous layer was re-extracted with EtOAc (10 mL). The combined organic layer was washed with saturated NaHCO₃ (2×30 mL) and brine (30 mL). The organic layer was dried over Na₂SO₄, filtered, and concentrated in vacuo to give a yellow solid. The solid was purified by column chromatography (silica gel; 0-10% MeOH:DCM) and then lyophilized to give 126 (2.20 g, 49.2% yield) as a yellow solid. (LCMS) m/z (M+H)⁺=533.4. ¹H NMR (400 MHz, (CD₃)₂SO) δ 8.45 (t, 1H), 8.25-8.21 (m, 2H), 7.64 (dd, 1H), 7.41-7.35 (m, 2H), 7.19-7.13 (m, 2H), 6.89 (d, 1H), 5.05 (d, 1H), 4.41-4.25 (m, 4H), 4.17-4.04 (m, 2H), 3.43-3.36 (m, 1H), 3.26-3.17 (m, 1H), 2.98 (br s, 6H), 2.90 (s, 6H), 1.38 (t, 3H), 1.20 (d, 3H). The chiral purity was determined using SFC analytical method on Chiralcel OD-3 50 mm×4.6 mm×3 μm column; Mobile phase A: CO₂/Mobile phase B: EtOH with 0.2% of NH₃; Isocratic: 40% Mobile phase B; backpressure: 1500 psi; flow rate: 3.5 mL/min; column temperature: 35° C. to provide 126: peak 1 ((SFC-MS) m/z (M+H)⁺=533.61 at retention time: 1.530 min, 100% ee). [α]²⁰_{D}=−121.791 (c=5.5 (g/L) in MeOH.

### Example 127

### (S)-5-(5-Dimethylcarbamoyl)pyrimidin-2-yl)-N-(4-(3,3-dimethylureido)benzyl)-1-isopropyl-7-methyl-4,5,6,7-tetrahydro-1H-pyrazolo[4,3-c]pyridine-3-carboxamide (127)

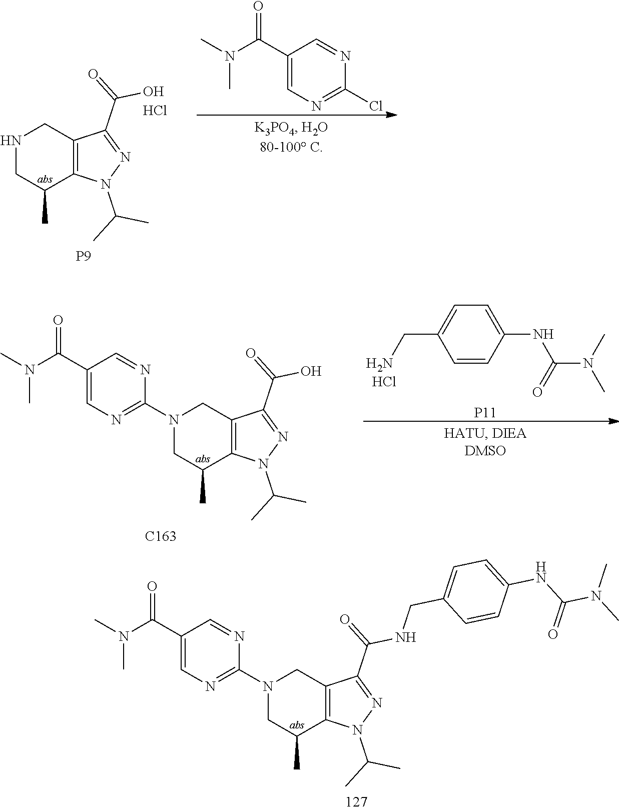

### Step 1. Preparation of (S)-5-(5-(dimethylcarbamoyl)pyrimidin-2-yl)-1-isopropyl-7-methyl-4,5,6,7-tetrahydro-1H-pyrazolo[4,3-c]pyridine-3-carboxylic acid (C163)

To a solution of 2-chloro-N,N-dimethylpyrimidine-5-carboxamide (CAS: 1637587-62-2; 3.86 g, 20.8 mmol) and P9 (5.40 g, 20.8 mmol) in H₂O (108 mL) was added K₃PO₄ (13.2 g, 62.4 mmol) at room temperature. The reaction mixture was heated between 80-100° C. for 18 h and then cooled to room temperature. The reaction mixture was extracted with EtOAc (50 mL×2), and the combined organic layer was discarded. The aqueous layer was acidified to pH=5-6 with aqueous HCl and then extracted with (1:10) MeOH:DCM (50 mL×4). The combined organic layer was dried over Na₂SO₄ and concentrated in vacuo to give C163 (7.10 g, crude) as a light yellow solid. The solid was used directly in the next step without further purification. (LCMS) m/z (M+H)⁺=373.2. ¹H NMR (400 MHz, (CD₃)₂SO) δ 12.73 (br s, 1H), 8.53 (s, 2H), 5.43 (d, 1H), 4.80-4.73 (m, 1H), 4.61-4.44 (m, 1H), 4.28 (d, 1H), 3.44-3.37 (m, 1H), 3.29-3.18 (m, 1H), 2.99 (br s, 6H), 1.40 (dd, 6H), 1.17-1.11 (m, 3H). The chiral purity was determined using SFC analytical method on Chiralpak AD-3 100 mm×4.6 mm×3 μm; Mobile phase A: CO₂/Mobile phase B: 40% EtOH with 0.2% of NH₃; backpressure: 1500 psi; flow rate: 2.8 mL/min; column temperature: 35° C. to provide C163: peak 1 ((SFC-MS) m/z (M+H)⁺=373.19 at retention time: 1.096 min, 100% ee).

### Step 2. Preparation of (S)-5-(5-(dimethylcarbamoyl)pyrimidin-2-yl)-N-(4-(3,3-dimethylureido)benzyl)-1-isopropyl-7-methyl-4,5,6,7-tetrahydro-1H-pyrazolo[4,3-c]pyridine-3-carboxamide (127)

To a solution of C163 (0.060 g, 0.16 mmol) in DMSO (1 mL) was added DIEA (0.062 g, 0.48 mmol), P11 (0.041 g, 0.18 mmol), and HATU (0.092 g, 0.24 mmol). The reaction was stirred at room temperature for 10 min and then was filtered. The filtrate was purified by reverse phase HPLC (Sunfire C₁₈ 100 mm×19 mm×5 μm column, Mobile Phase A: H₂O (0.05% TFA)/Mobile Phase B: ACN, 15 to 95% of Mobile Phase B over 9.0 min, 95% of Mobile Phase B hold 1.0 min, flow rate: 25 mL/min) to provide 127 (15 mg, 17% yield) as a solid. (LCMS) m/z (M+H)⁺=548.9. ¹H NMR (400 MHz, (CD₃)₂SO) δ 8.52 (s, 2H), 8.34 (t, 1H), 8.23 (s, 1H), 7.41-7.36 (m, 2H), 7.19-7.14 (m, 2H), 5.47 (d, 1H), 4.75 (dd, 1H), 4.58-4.46 (m, 1H), 4.40-4.29 (m, 3H), 3.42 (dd, 1H), 3.29-3.23 (m, 1H) 3.04-2.94 (m, 6H), 2.91 (s, 6H), 1.38 (dd, 6H), 1.11 (d, 3H). The chiral purity was determined using SFC analytical method on Chiralpak AD-3 50 mm×4.6 mm×3 μm; Mobile phase A: CO₂/Mobile phase B: isopropyl alcohol with 0.2% NH₃; Isocratic: 40% Mobile phase B; backpressure: 1500 psi; flow rate: 3.5 mL/min; column temperature: 35° C. to provide 127: peak 1 ((SFC-MS) m/z (M+H)⁺=548.70 at retention time: 1.882 min, 100% ee). [α]²⁰_{D}=−149.239 (c=1.5 (g/L) in MeOH).

### Example 128

### (S)-5-(5-(Dimethylcarbamoyl)pyridin-2-yl)-N-(4-(3,3-dimethylureido)benzyl)-1-isopropyl-7-methyl-4,5,6,7-tetrahydro-1H-pyrazolo[4,3-c]pyridine-3-carboxamide (128)

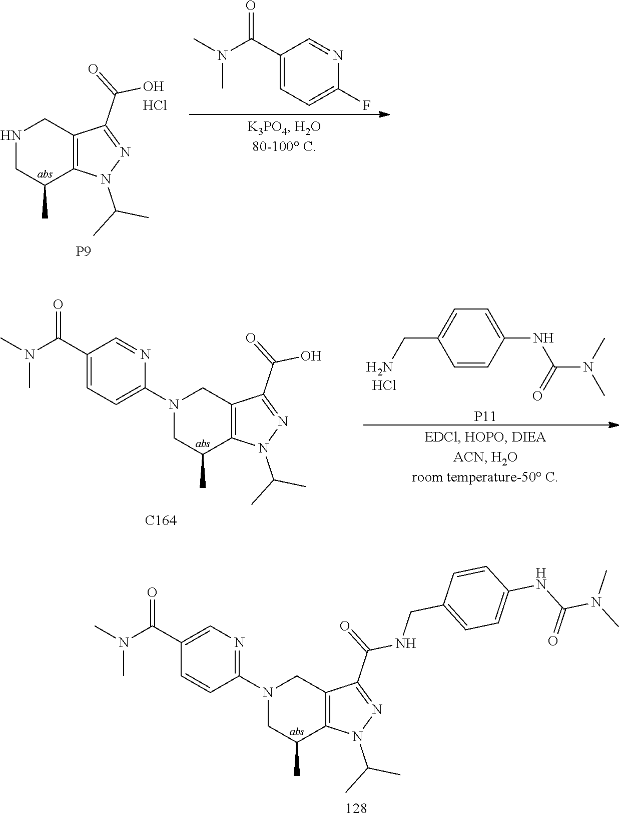

### Step 1. Preparation of (S)-5-(5-(dimethylcarbamoyl)pyridin-2-yl)-1-isopropyl-7-methyl-4,5,6,7-tetrahydro-1H-pyrazolo[4,3-c]pyridine-3-carboxylic acid (C164)

To a solution of 6-fluoro-N,N-dimethylnicotinamide (CAS: 1032251-82-3; 2.91 g, 17.3 mmol) and P9 (5.00 g, 19.2 mmol) in H₂O (100 mL) was added K₃PO₄ (12.3 g, 57.7 mmol) at room temperature. The reaction mixture was stirred between 80-100° C. for 18 h and then cooled to room temperature. To the suspension was added another portion of 6-fluoro-N,N-dimethylnicotinamide (CAS: 1032251-82-3; 0.227 g, 1.35 mmol), then the reaction mixture was stirred between 80-100° C. for 48 h. The suspension was cooled to room temperature and extracted with EtOAc (100 mL×4). The combined organic layer was discarded. The aqueous layer was acidified to pH=5-6 with aqueous HCl then extracted with (1:10) MeOH:DCM (50 mL×9). The combined organic layer was dried over Na₂SO₄ and concentrated in vacuo to give C164 (5.80 g, crude) as a light yellow solid. The solid was used directly in the next step without further purification. (LCMS) m/z (M+H)⁺=372.1. ¹H NMR (400 MHz, (CD₃)₂SO) δ 12.70 (br s, 1H), 8.26-8.24 (m, 1H), 7.64 (dd, 1H), 6.91 (d, 1H), 5.08 (d, 1H), 4.60-4.49 (m, 1H), 4.43-4.36 (m, 1H), 4.19 (d, 1H), 3.37-3.35 (m, 1H), 3.26-3.21 (m, 1H), 2.98 (s, 6H), 1.40 (dd, 6H), 1.20 (d, 3H). The chiral purity was determined using SFC analytical method on Chiralpak AD-3 100 mm×4.6 mm×3 μm; Mobile phase A: CO₂/Mobile phase B: 40% EtOH with 0.2% of NH₃; backpressure: 1500 psi; flow rate: 2.8 mL/min; column temperature: 35° C. to provide C164: peak 1 ((SFC-MS) m/z (M+H)⁺=372.17 at retention time: 1.254 min, 100% ee).

### Step 2. Preparation of (S)-5-(5-(dimethylcarbamoyl)pyridin-2-yl)-N-(4-(3,3-dimethylureido)benzyl)-1-isopropyl-7-methyl-4,5,6,7-tetrahydro-1H-pyrazolo[4,3-c]pyridine-3-carboxamide (128)

To a solution of C164 (3.20 g, 8.61 mmol), EDCl (2.06 g, 10.8 mmol), and HOPO (1.20 g, 10.8 mmol) in ACN (50 mL) was added DIEA (4.45 g, 34.4 mmol). The solution was stirred at 50° C. for 2 h, which caused precipitate to form. The reaction mixture was cooled to room temperature, then P11 (2.18 g, 9.47 mmol), H₂O (0.155 g, 8.61 mmol), and an additional portion of DIEA (4.45 g, 34.4 mmol) were added. The reaction mixture was stirred at 50° C. for 6 h and then diluted with H₂O (20 mL). The suspension was concentrated in vacuo to remove the solvent and leave the aqueous layer. The aqueous layer was extracted with (1:10) MeOH:DCM (3×70 mL). The combined organic layer was washed with saturated NaHCO₃ (15 mL) and brine (15 mL). The organic layer was dried over Na₂SO₄, filtered, and concentrated in vacuo to give a yellow solid. The yellow solid was purified by column chromatography (silica gel; 0-10% MeOH:DCM) and then lyophilized to give 128 (1.75 g, 37.2% yield) as a white solid. (LCMS) m/z (M+H)⁺=547.3. ¹H NMR (400 MHz, (CD₃)₂SO) δ 8.29 (t, 1H), 8.18-8.16 (m, 2H), 7.57 (dd, 1H), 7.34-7.29 (m, 2H), 7.14-7.08 (m, 2H), 6.82 (d, 1H), 5.03 (d, 1H), 4.50-4.41 (m, 1H), 4.39-4.23 (m, 3H), 4.17 (d, 1H), 3.36-3.35 (m, 1H), 3.18-3.12 (m, 1H), 2.91 (s, 6H), 2.84 (s, 6H), 1.35 (dd, 6H), 1.13 (d, 3H). The chiral purity was determined using SFC analytical method on Chiralcel OD-3 50 mm×4.6 mm×3 μm; Mobile phase A: CO₂/Mobile phase B: EtOH with 0.2% NH₃; Isocratic 40% of Mobile phase B; backpressure: 1500 psi; flow rate: 4.0 mL/min; column temperature: 35° C. to provide 128: peak 1 ((SFC-MS) m/z (M+H)⁺=547.54 at retention time: 1.035 min, 99% ee). [α]²⁵_{D}=−12.573 (c=2.6 (g/L) in MeOH).

### Example 132

### (S)-5-(4-(Dimethylcarbamoyl)phenyl)-1-isopropyl-7-methyl-N-(4-(4-methylpiperazine-1-carboxamido)benzyl)-4,5,6,7-tetrahydro-1H-pyrazolo[4,3-c]pyridine-3-carboxamide (132)

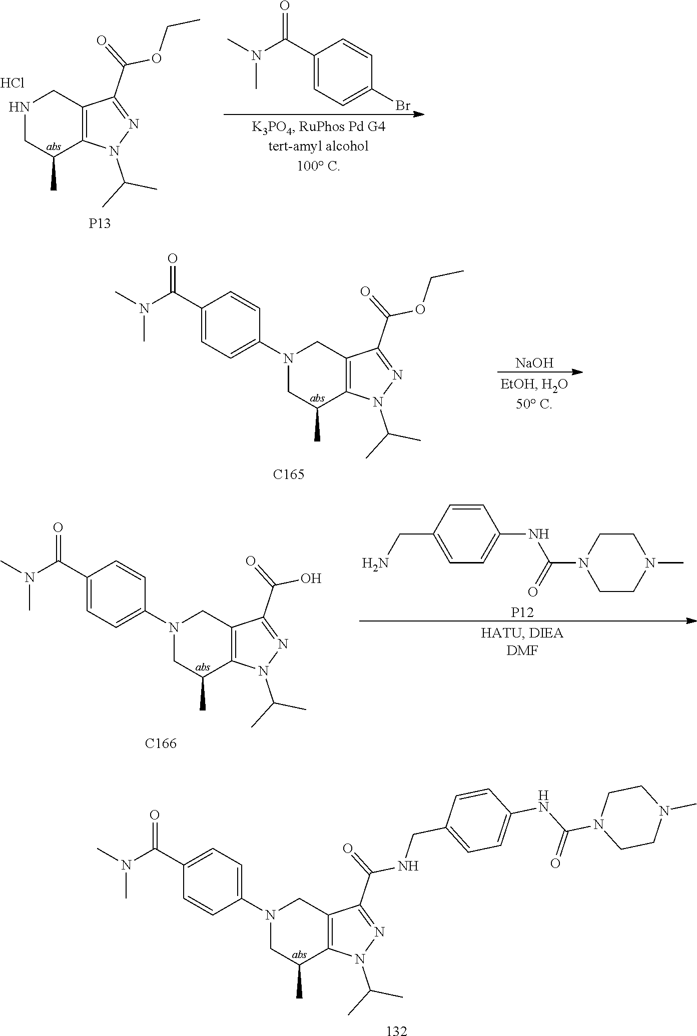

### Step 1. Preparation of ethyl (S)-5-(4-(dimethylcarbamoyl)phenyl)-1-isopropyl-7-methyl-4,5,6,7-tetrahydro-1H-pyrazolo[4,3-c]pyridine-3-carboxylate (C165)

A reaction mixture of 4-bromo-N,N-dimethylbenzamide (0.344 g, 1.51 mmol), K₃PO₄ (0.667 g, 3.14 mmol) and RuPhos Pd G4 (0.107 g, 0.126 mmol) was degassed with nitrogen gas before a solution of P13 (0.362 g, 1.26 mmol) in tert-amyl alcohol (3 mL) was added. The suspension was stirred at 100° C. for 25 h, then filtered and concentrated in vacuo. The residue was dissolved in DCM and purified by column chromatography (silica gel, 20-100% EtOAc:heptane) to provide C165 (0.512 g, crude) as a yellow oil. The oil was used directly in the next step without further purification. (LC/MS) m/z (M+H)⁺=399.6. ¹H NMR (600 MHz, (CD₃)₂SO) δ 7.38-7.34 (m, 2H), 7.03-6.98 (m, 2H), 4.64-4.56 (m, 1H), 4.53 (d, 1H), 4.31 (q, 2H), 4.11 (d, 1H), 3.88-3.83 (m, 1H), 3.32-3.22 (m, 2H), 2.98 (s, 6H), 1.43 (dd, 6H), 1.34 (t, 3H), 1.31 (d, 3H).

### Step 2. Preparation of (S)-5-(4-(dimethylcarbamoyl)phenyl)-1-isopropyl-7-methyl-4,5,6,7-tetrahydro-1H-pyrazolo[4,3-c]pyridine-3-carboxylic acid (C166)

To a solution of C165 (0.500 g, 1.25 mmol) in EtOH (3 mL) was added NaOH (0.151 g, 3.76 mmol) and H₂O (1 mL). The reaction mixture was stirred at 50° C. for 25 h, then acidified with HCl and concentrated in vacuo to provide C166 (0.465 g, crude) as a yellow solid. The solid was used directly in the next step without further purification. (LC/MS) m/z (M+H)⁺=371.4. ¹H NMR (600 MHz, (CD₃)₂SO) δ 7.37-7.31 (m, 2H), 7.01-6.95 (m, 2H), 4.61-4.52 (m, 1H), 4.50 (d, 1H), 4.11-4.05 (m, 1H), 3.82 (dd, 1H), 3.29 (dd, 1H), 3.24-3.18 (m, 1H), 2.96 (s, 6H), 1.44-1.39 (m, 6H), 1.29 (d, 3H).

### Step 3. Preparation of (S)-5-(4-(dimethylcarbamoyl)phenyl)-1-isopropyl-7-methyl-N-(4-(4-methylpiperazine-1-carboxamido)benzyl)-4,5,6,7-tetrahydro-1H-pyrazolo[4,3-c]pyridine-3-carboxamide (132)

To a solution of C166 (37 mg, 0.10 mmol) and P12 (32 mg, 0.10 mmol) in DMF (1 mL) was added HATU (38 mg, 0.10 mmol). The suspension was stirred at room temperature for 2 min, and then DIEA (64 mg, 0.50 mmol) was added. The yellow solution was stirred at room temperature for 2 h, then diluted with MeOH and purified by reverse phase HPLC (Phenomenex Gemini NX C₁₈ 150 mm×30 mm×5 μm column, Mobile Phase A: H₂O (NH₄OH—NH₄HCO₃)/Mobile Phase B: ACN, 26-66% of Mobile Phase B over 9 min, 100% of Mobile Phase B hold for 2 min, flow rate: 60 mL/min) and lyophilized to provide 132 (0.020 g, 34% yield) as a white solid. (LCMS) m/z (M+H)⁺=601.3. ¹H NMR (400 MHz, (CD₃)₂SO) δ 8.46 (s, 1H), 8.36 (t, 1H), 7.40-7.28 (m, 4H), 7.21-7.12 (m, 2H), 7.00-6.91 (m, 2H), 4.59-4.48 (m, 2H), 4.36-4.30 (m, 2H), 4.10 (d, 1H), 3.85-3.77 (m, 1H), 3.41 (t, 4H), 3.28-3.16 (m, 2H), 2.97-2.91 (m, 6H), 2.29 (t, 4H), 2.18 (s, 3H), 1.47-1.37 (m, 6H), 1.28 (d, 3H). The chiral purity was determined using SFC analytical method on Chiralcel OD-3 50 mm×4.6 mm×3 μm column; Mobile phase A: CO₂/Mobile phase B: EtOH (0.2% NH₃); 5 to 40% of Mobile phase B in 1.5 min and held at 40% of Mobile phase B for 1.0 min then 5% of Mobile phase B for 0.5 min; backpressure: 1500 psi; flow rate: 4.0 mL/min; column temperature: 35° C. to provide peak 1: ((SFC-MS) m/z (M+H)⁺=601.32 at retention time: 1.830 min, 0.27% ee) and 132: peak 2 ((SFC-MS) m/z (M+H)⁺=601.31 at retention time: 2.105 min, 99.73% ee).

### Example 136

### 5-(5-(Dimethylcarbamoyl)pyridin-2-yl)-1-isopropyl-N-(4-(pyrrolidine-1-carboxamido)benzyl)-4,5,6,7-tetrahydro-1H-pyrazolo[4,3-c]pyridine-3-carboxamide (136)

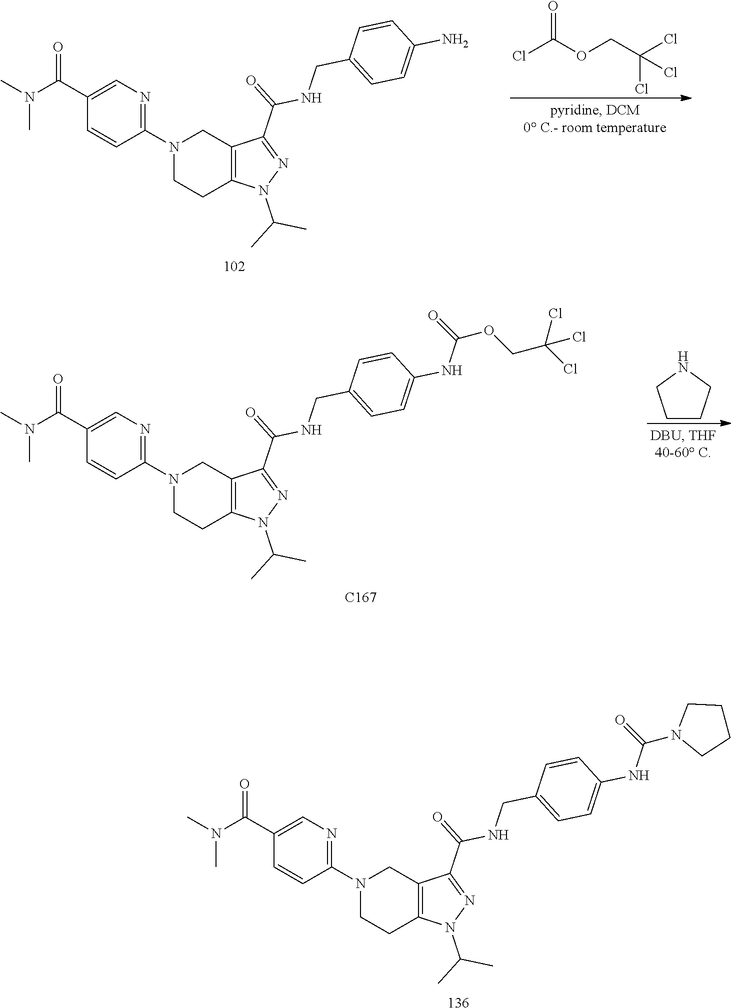

### Step 1. Preparation of 2,2,2-trichloroethyl (4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)carbamate (C167)

At 0° C., to a solution of 102 (1.3 g, 2.8 mmol) in DCM (13 mL) was added pyridine (0.49 g, 6.2 mmol) followed by a solution of 2,2,2-trichloroethoxycarbonylchloride (0.77 g, 3.6 mmol) in DCM (1 mL) dropwise. The reaction mixture was stirred for 30 min at 0° C., then warmed to room temperature and concentrated in vacuo. The residue was dissolved in MeOH:DCM then purified by column chromatography (silica gel, 0-10% MeOH:DCM) to provide C167 (1.7 g, 93% yield) as a white foam. (LCMS) m/z (M+H)⁺=638.6.

### Step 2. Preparation of 5-(5-(dimethylcarbamoyl)pyridin-2-yl)-1-isopropyl-N-(4-(pyrrolidine-1-carboxamido)benzyl)-4,5,6,7-tetrahydro-1H-pyrazolo[4,3-c]pyridine-3-carboxamide (136)

In a gold para-Dox® plate was added a reaction mixture of pyrrolidine (0.070 mL) and DBU (0.030 mL) in THF (0.25 mL) and then shaken at 40° C. for 2 h. A solution of C167 (6.4 mg, 0.010 mmol) in THF (0.023 mL) was added to the suspension, and then the reaction mixture was sealed and shaken at 60° C. overnight. The suspension was dissolved in (1:1, MeOH:DCE, 0.35 mL) and then filtered. The filter cake was rinsed with (1:1, MeOH:DCE, 0.35 mL×2). The filtrate was concentrated in vacuo and then dissolved in (1:1, MeOH:DCE, 0.20 mL) and shaken at room temperature for 2 h. The solution was purified by reverse phase HPLC (C₁₈ 50 mm×10 mm×5 μm column; Mobile Phase A: H₂O/Mobile Phase B: ACN (0.05% formic acid), 5 to 95% of Mobile Phase B over 5 min, flow rate: 4.0 mL/min) to provide 136 (1.1 mg, 20% yield). (LCMS) m/z (M+H)⁺=559.4.

### Example 193

### (S)-5-(5-(3-Acetamidopyrrolidine-1-carbonyl)pyridin-2-yl)-N-(4-(3,3-dimethylureido)benzyl)-1-isopropyl-4,5,6,7-tetrahydro-1H-pyrazolo[4,3-c]pyridine-3-carboxamide (193)

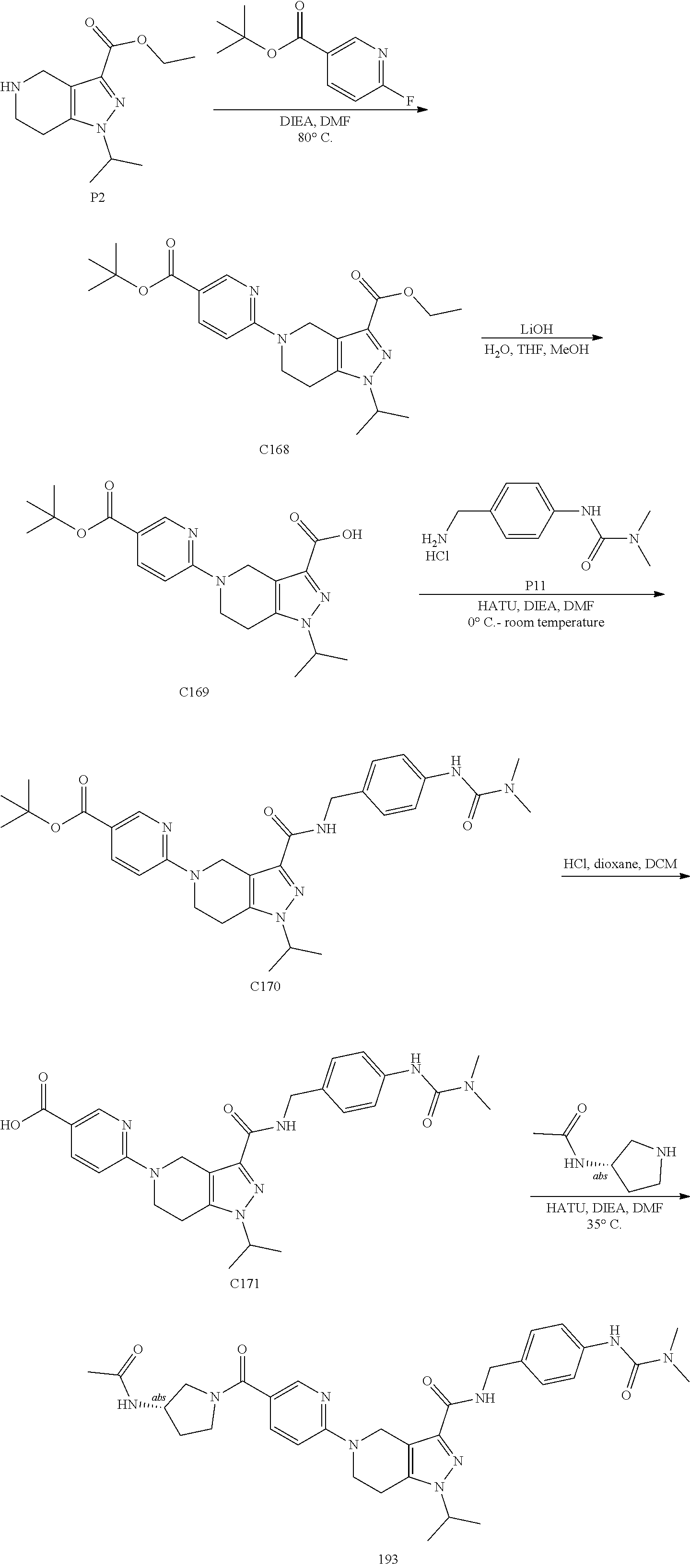

### Step 1. Preparation of ethyl 5-(5-(tert-butoxycarbonyl)pyridin-2-yl)-1-isopropyl-4,5,6,7-tetrahydro-1H-pyrazolo[4,3-c]pyridine-3-carboxylate (C168)

A suspension of P2 (1.85 g, 6.76 mmol) in DMF (40 mL) was added DIEA (2.62 g, 20.3 mmol) and tert-butyl 6-fluoronicotinate (CAS: 676560-01-3, 1.33 g, 6.76 mmol). The reaction mixture was stirred at 80° C. for 16 h, and then an additional portion of tert-butyl 6-fluoronicotinate (CAS: 676560-01-3,133 mg, 0.676 mmol) was added. The reaction mixture was stirred at 80° C. for 4 h and then was diluted with H₂O (300 mL). The suspension was extracted with EtOAc (3×100 mL). The combined organic layer was washed with brine (2×100 mL), dried with Na₂SO₄, filtered, and concentrated in vacuo. The residue was purified by column chromatography (silica gel, 0-25% EtOAc:PE) to provide C168 (1.86 g, 66.4%) as a white solid.

### Step 2. Preparation of 5-(5-(tert-butoxycarbonyl)pyridin-2-yl)-1-isopropyl-4,5,6,7-tetrahydro-1H-pyrazolo[4,3-c]pyridine-3-carboxylic acid (C169)

The same reaction was conducted in two batches and then combined for acidification. To a solution of C168 (1.76 g, 4.25 mmol) in THF (10 mL), MeOH (5 mL) and H₂O (10 mL) was added LiOH (0.445 g, 10.6 mmol) to form the first batch then stirred for 16 h.

A second batch of the same reaction was conducted with C168 (0.100 g, 0.241 mmol). The two batches were combined and then concentrated in vacuo. The residue was diluted with H₂O (15 mL) and acidified with 1M HCl to pH˜4-5, which caused precipitate to form. The suspension was filtered, and the filter cake was washed with H₂O (2×10 mL). The filter cake was collected and dried by high vacuum to provide C169 (1.73 g, crude) as an off-white solid. The solid was used directly in the next step without further purification. (LCMS) m/z (M+H)⁺=387.1.

### Step 3. Preparation of tert-butyl 6-(3-((4-(3,3-dimethylureido)benzyl)carbamoyl)-1-isopropyl-1,4,6,7-tetrahydro-5H-pyrazolo[4,3-c]pyridin-5-yl)nicotinate (C170)

At 0° C., to a solution of C169 (1.73 g, 4.49 mmol) in DMF (30 mL) was added HATU (1.88 g, 4.94 mmol) and stirred for 20 min; then, DIEA (2.32 g, 17.9 mmol) and P11 (1.13 g, 4.94 mmol) were added. The reaction mixture was stirred at room temperature for 16 h and then diluted with H₂O (200 mL). The suspension was extracted with (1:10, MeOH:DCM, 3×100 mL). The combined organic layer was washed with brine (2×150 mL), dried with Na₂SO₄, filtered, and concentrated in vacuo. The residue was purified by column chromatography (silica gel, 0-5% MeOH:DCM) to provide impure C170 (4.00 g), which was re-purified again by column chromatography (silica gel, 0-2.5% MeOH:DCM) to provide C170 (3.5 g, >95.0% yield) as a white solid. (LCMS) m/z (M+H)⁺=562.2. ¹H NMR (400 MHz, (CD₃)₂SO) δ 8.62 (d, 1H), 8.35 (t, 1H), 8.26-8.21 (m, 1H), 7.92 (dd, 1H), 7.44-7.36 (m, 2H), 7.21-7.14 (m, 2H), 6.91 (d, 1H), 4.80 (s, 2H), 4.54-4.43 (m, 1H), 4.35 (d, 2H), 4.04-3.94 (m, 2H), 2.96-2.89 (m, 6H), 2.86-2.79 (m, 2H), 1.52 (s, 9H), 1.40 (d, 6H).

### Step 4. Preparation of 6-(3-((4-(3,3-dimethylureido)benzyl)carbamoyl)-1-isopropyl-1,4,6,7-tetrahydro-5H-pyrazolo[4,3-c]pyridin-5-yl)nicotinic acid (C171)

To a solution of C170 (3.50 g, 6.23 mmol) in DCM (70 mL) was added 2M HCl in dioxane (35 mL) and then stirred for 16 h, and an additional portion of 2M HCl in dioxane (30 mL) was added. The reaction mixture was stirred for 2 h and then was diluted with H₂O (20 mL) and concentrated in vacuo to remove the organic layer. The aqueous layer was basified with NaHCO₃ until pH=5, which caused white precipitation to form. The mixture was filtered, and then the filter cake was washed with H₂O (2×30 mL), dried under a high vacuum, and then lyophilized to provide C171 (2.22 g, 69.8% yield) as a white solid (LCMS) m/z (M+H)⁺=506.4. ¹H NMR (400 MHz, (CD₃)₂SO) δ 12.51 (s, 1H), 8.65 (d, 1H), 8.35 (t, 1H), 8.23 (s, 1H), 7.96 (dd, 1H), 7.42-7.36 (m, 2H), 7.20-7.14 (m, 2H), 6.91 (d, 1H), 4.80 (s, 2H), 4.58-4.43 (m, 1H), 4.34 (d, 2H), 4.04-3.97 (m, 2H), 2.91 (s, 6H), 2.86-2.80 (m, 2H), 1.39 (d, 6H).

### Step 5. Preparation of (S)-5-(5-(3-acetamidopyrrolidine-1-carbonyl)pyridin-2-yl)-N-(4-(3,3-dimethylureido)benzyl)-1-isopropyl-4,5,6,7-tetrahydro-1H-pyrazolo[4,3-c]pyridine-3-carboxamide (193)

A solution of (S)—N-(pyrrolidin-3-yl)acetamide (CAS: 114636-31-6; 1.9 mg, 15 μmol) in (1:9, DIEA:DMF, 5.0 μL:45 μL) was shaken at 30° C. for 1.5 h.

In a gold para-Dox® plate, a solution of C171 (66 mg, 0.13 mmol), HATU (74 mg, 0.19 mmol), and DIEA (0.065 mL) in DMF (0.58 mL) was stirred for 20 min and then shaken at 35° C. for 19 h. The solution was concentrated under nitrogen gas. The residue was dissolved in (1:1, MeOH:DCE) and then shaken at 30° C. for 10 min. The suspension was filtered, and then the filter cake was rinsed with (1:1, MeOH:DCE, 0.60 mL). The filtrate was concentrated with nitrogen gas. The residue was dissolved in DMSO (0.15 mL) and then shaken at 30° C. for 30 min. TFA (2.0 μL) and H₂O (0.50 mL) were added to the suspension. The suspension was concentrated with nitrogen gas then dissolved in DMSO (0.15 mL) and purified by reverse phase HPLC (C18 50 mm×10 mm×5 μm column; Mobile Phase A: H₂O/Mobile Phase B: ACN (0.05% formic acid), 50 to 80% of Mobile Phase B over 5 min, flow rate: 4.0 mL/min) to provide 193 (2.1 mg, 34% yield). (LCMS) m/z (M+H)⁺=616.4.

### Example 236

### Ethyl (4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)carbamate (236)

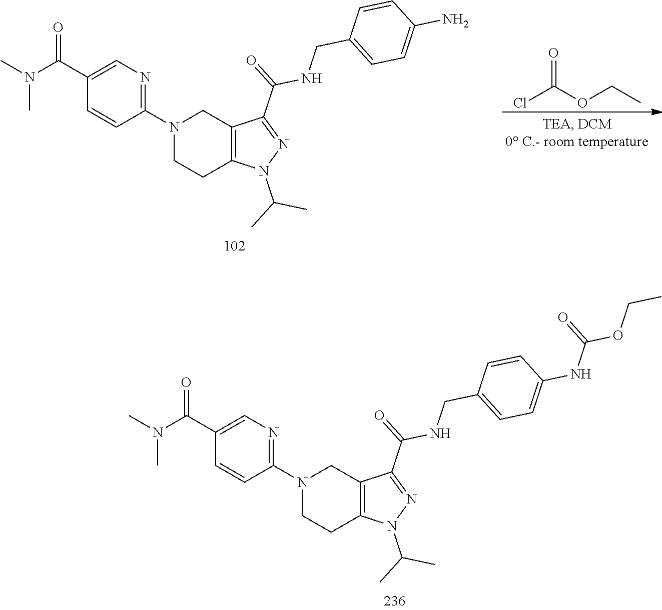

### Step 1. Preparation of ethyl (4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)carbamate (236)

At 0° C., to a reaction mixture of 102 (0.200 g, 0.433 mmol) in DCM (2 mL) was added TEA (0.132 g, 1.30 mmol), then a solution of ethyl carbonochloridate (CAS: 541-41-3; 0.310 g, 2.86 mmol) in DCM (1 mL) dropwise. The reaction mixture was sealed and stirred at room temperature for 16 h. The suspension was diluted with H₂O (10 mL) and stirred at 0° C. for 30 min, and then the mixture's pH was adjusted to pH=8. The reaction mixture was extracted with DCM (2×10 mL). The organic layers were combined, dried with Na₂SO₄, and concentrated in vacuo. The residue was purified by reverse phase HPLC (Welch Xtimate C₁₈ 150 mm×25 mm×5 μm column, Mobile Phase A: H₂O (NH₄OH—NH₄HCO₃)/Mobile Phase B: ACN, 38 to 58% of Mobile Phase B over 11 min, 100% of Mobile Phase B hold 2 min, flow rate: 60 mL/min) and lyophilized to provide 236 (84.0 mg, 36.4% yield) as a white solid. (LCMS) m/z (M+H)⁺=534.3. ¹H NMR (400 MHz, (CD₃)₂SO) δ 9.46 (s, 1H), 8.28 (t, 1H), 8.16-8.14 (m, 1H), 7.55 (dd, 1H), 7.30 (d, 2H), 7.14 (d, 2H), 6.80 (d, 1H), 4.66 (s, 2H), 4.46-4.36 (m, 1H), 4.27 (d, 2H), 4.02 (q, 2H), 3.86 (t, 2H), 2.88 (s, 6H), 2.73 (t, 2H), 1.31 (d, 6H), 1.14 (t, 3H).

### Example 252

### 6-(10-(4-(3,3-Dimethylureido)benzyl)-11-oxo-1,3,4,7,8,9,10,11-octahydro-2H-pyrido[4′,3′:3,4]pyrazolo[1,5-a][1,4]diazepin-2-yl)-N,N-dimethylnicotinamide (252)

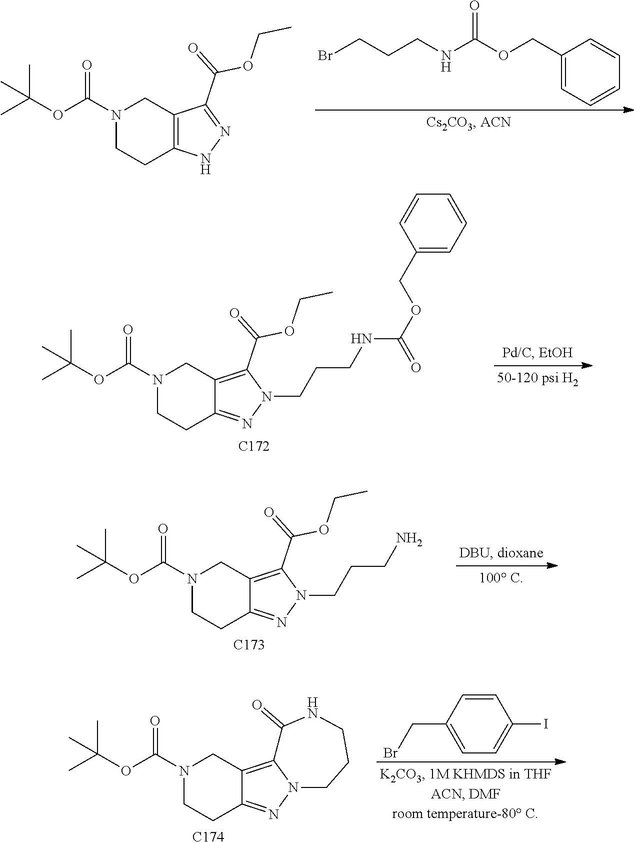

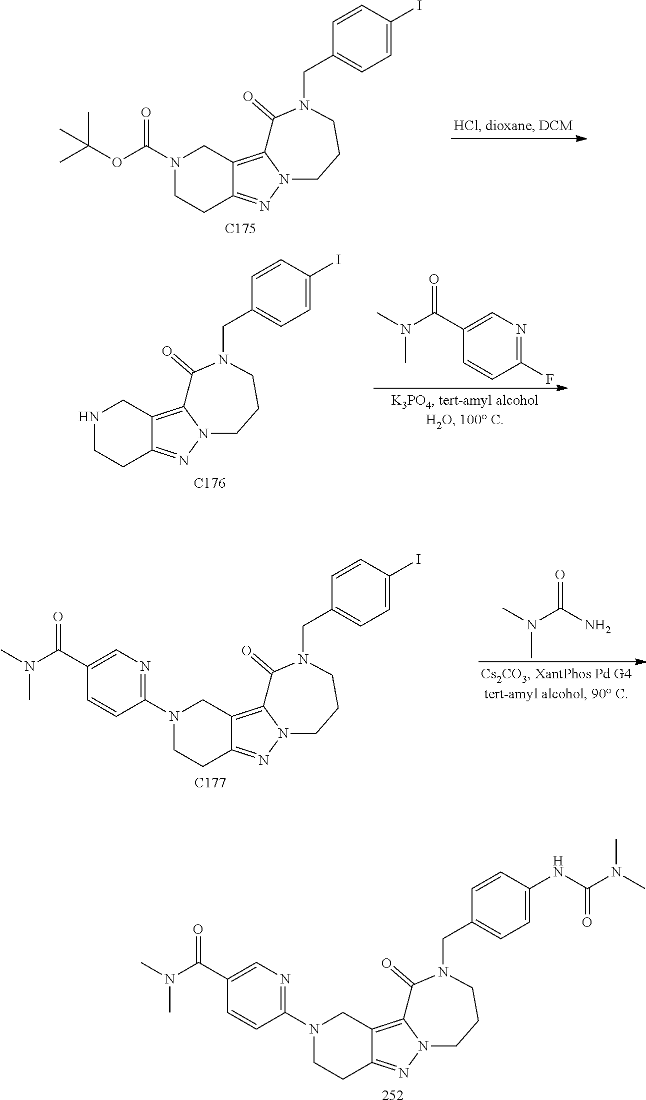

### Step 1. Preparation of 5-(tert-butyl) 3-ethyl 2-(3-(((benzyloxy)carbonyl)amino)propyl)-2,4,6,7-tetrahydro-5H-pyrazolo[4,3-c]pyridine-3,5-dicarboxylate (C172)

A reaction mixture of 5-(tert-butyl) 3-ethyl 1,4,6,7-tetrahydro-5H-pyrazolo[4,3-c]pyridine-3,5-dicarboxylate (CAS: 518990-23-3; 1.00 g, 3.39 mmol), benzyl (3-bromopropyl)carbamate (CAS: 39945-54-5; 967 mg, 3.56 mmol) and Cs₂CO₃ (1.65 g, 5.08 mmol) in ACN (20 mL) was stirred at room temperature for ˜72 h. The reaction mixture was filtered, and the filtrate was concentrated in vacuo. The residue was dissolved in DCM and then purified by column chromatography (silica gel, 0-60% EtOAc:heptane) to provide C172 (1.02 g, 61.9% yield) as a clear oil. (LCMS) m/z (M+H)⁺=487.3. ¹H NMR (600 MHz, (CD₃)₂SO) δ 7.41-7.26 (m, 6H), 5.01 (s, 2H), 4.53 (s, 2H), 4.45 (t, 2H), 4.32-4.28 (m, 2H), 3.60 (t, 2H), 3.01 (q, 2H), 2.64 (t, 2H), 1.93-1.85 (m, 2H), 1.44-1.41 (m, 9H), 1.32 (t, 3H).

### Step 2. Preparation of 5-(tert-butyl) 3-ethyl 2-(3-aminopropyl)-2,4,6,7-tetrahydro-5H-pyrazolo[4,3-c]pyridine-3,5-dicarboxylate (C173)

Pd/C (10 wt %, 0.153 g, 1.44 mmol) and C172 (1.00 g, 2.06 mmol) were added to a pressure vessel in EtOH (10 mL). The reaction mixture was stirred at 50 psi of H₂ gas for ˜24 h. An additional portion of Pd/C (10 wt %, 0.153 g, 1.44 mmol) was added and stirred at 120 psi of H₂ gas for ˜24 h. The reaction mixture was filtered through celite, and then the filtrate was concentrated in vacuo to provide C173 (0.745 g, crude) as a clear oil. The oil was used directly in the next step without further purification. ¹H NMR (400 MHz, (CD₃)₂SO) δ 4.55-4.41 (m, 3H), 4.37-4.26 (m, 3H), 3.60 (t, 2H), 3.50-3.40 (m, 2H), 2.64 (t, 2H), 2.57-2.52 (m, 2H), 1.92-1.77 (m, 2H), 1.44-1.41 (m, 9H), 1.33 (t, 3H).

### Step 3. Preparation of tert-butyl 11-oxo-1,3,4,7,8,9,10,11-octahydro-2H-pyrido[4′,3′:3,4]pyrazolo[1,5-a][1,4]diazepine-2-carboxylate (C174)

To a solution of C173 (0.724 g, 2.05 mmol) in dioxane (10 mL) was added DBU (0.109 g, 0.719 mmol) and then stirred at 100° C. for 23 h. The reaction mixture was concentrated in vacuo to provide C174 (0.820 g, crude) as a yellow oil. The oil was used directly in the next step without further purification. ¹H NMR (400 MHz, (CD₃)₂SO) δ 7.89 (s, 1H), 4.24-4.20 (m, 2H), 4.09 (t, 2H), 3.03-2.87 (m, 4H), 2.42-2.33 (m, 2H), 1.91-1.83 (m, 2H), 1.19-1.13 (m, 9H).

### Step 4. Preparation of tert-butyl 10-(4-iodobenzyl)-11-oxo-1,3,4,7,8,9,10,11-octahydro-2H-pyrido[4′,3′:3,4]pyrazolo[1,5-a][1,4]diazepine-2-carboxylate (C175)

To a solution of C174 (0.629 g, 2.05 mmol) in ACN (10 mL) was added 1-(bromomethyl)-4-iodobenzene (CAS: 589-87-7; 0.610 g, 2.05 mmol) and K₂CO₃ (426 mg, 3.08 mmol). The reaction mixture was stirred at 80° C. for 2 h and dissolved in THF (10 mL). The solution was treated with 1M KHMDS in THF (2.0 mL) at room temperature and then diluted with DMF (1 mL). The reaction mixture was stirred at room temperature for 2.5 h and then concentrated in vacuo. The residue was suspended with H₂O and extracted with EtOAc. The organic layer was concentrated in vacuo, and then the residue was dissolved in DCM. The suspension was purified by column chromatography (silica gel, 0-80% EtOAc:heptane) to provide C175 (0.496 g, 46.3% yield) as a white foam. ¹H NMR (400 MHz, (CD₃)₂SO) δ 7.76-7.64 (m, 2H), 7.22-7.13 (m, 2H), 4.62 (s, 2H), 4.47 (s, 2H), 4.26 (t, 2H), 3.60 (t, 2H), 3.39-3.34 (m, 2H), 2.62 (t, 2H), 2.07-1.98 (m, 2H), 1.42 (s, 9H).

### Step 5. Preparation of 10-(4-iodobenzyl)-1,2,3,4,7,8,9,10-octahydro-11H-pyrido[4′,3′:3,4]pyrazolo[1,5-a][1,4]diazepin-11-one hydrochloride (C176)

To a solution of C175 (0.496 g, 0.949 mmol) in DCM (10 mL) was added 4M HCl in dioxane (0.95 mL). The reaction mixture was concentrated in vacuo to provide C176 (0.436 g, crude) as a white solid. The solid was used directly in the next step without further purification. (LCMS) m/z (M+H)⁺=423.3. ¹H NMR (400 MHz, (CD₃)₂SO) δ 9.12 (s, 1H), 7.75-7.69 (m, 2H), 7.21-7.14 (m, 2H), 4.65-4.60 (m, 2H), 4.35-4.29 (m, 2H), 4.27-4.21 (m, 2H), 3.43-3.34 (m, 4H), 2.91-2.83 (m, 2H), 2.12-2.01 (m, 2H).

### Step 6. Preparation of 6-(10-(4-iodobenzyl)-11-oxo-1,3,4,7,8,9,10,11-octahydro-2H-pyrido[4′,3′:3,4]pyrazolo[1,5-a][1,4]diazepin-2-yl)-N,N-dimethylnicotinamide (C177)

To a mixture of C176 (0.436 g, 0.950 mmol), K₃PO₄ (0.605 g, 2.85 mmol), H₂O (4 mL), and tert-amyl alcohol (4 mL), 6-fluoro-N,N-dimethylnicotinamide (CAS: 1032251-82-3; 176 mg, 1.05 mmol) was added. The reaction mixture was stirred at 100° C. for ˜72 h, diluted with H₂O, and extracted with EtOAc. The organic layer was washed with H₂O and concentrated in vacuo to provide C177 (0.632 g, crude) as a yellow oil. The oil was used directly in the next step without further purification.

### Step 7. Preparation of 6-(10-(4-(3,3-dimethylureido)benzyl)-11-oxo-1,3,4,7,8,9,10,11-octahydro-2H-pyrido[4′,3′:3,4]pyrazolo[1,5-a][1,4]diazepin-2-yl)-N,N-dimethylnicotinamide (252)

A reaction mixture of C177 (0.080 g, 0.14 mmol), 1,1-dimethylurea (CAS: 598-94-7; 37 mg, 0.42 mmol), Cs₂CO₃ (0.14 g, 0.42 mmol) and XantPhos Pd G4 (13 mg, 0.014 mmol) was degassed with nitrogen gas then tert-amyl alcohol (1 mL) was added. The suspension was stirred at 90° C. for 2 h and then filtered, and the filtrate was concentrated in vacuo. The residue was suspended in DMSO, filtered and purified by reverse phase HPLC (Sunfire C₁₈ 100 mm×19 mm×5 μm column, Mobile Phase A: H₂O/Mobile Phase B: ACN (0.05% TFA), 5 to 95% of Mobile Phase B over 10 min, flow rate: 25 mL/min) to provide 252 (4.6 mg, 6.2% yield). (LC/MS) m/z (M+H)⁺=531.4. ¹H NMR (600 MHz, (CD₃)₂SO) δ 8.28 (s, 1H), 8.24 (d, 1H), 7.64 (dd, 1H), 7.48-7.44 (m, 2H), 7.27-7.23 (m, 2H), 6.92 (d, 1H), 4.74 (s, 2H), 4.60 (s, 2H), 4.24 (t, 2H), 3.94 (t, 2H), 3.33 (t, 2H), 2.97 (s, 6H), 2.92 (s, 6H), 2.73 (t, 2H), 1.99-1.92 (m, 2H).

### Example 286

### 5-(5-(2-(Dimethylamino)-2-oxoethyl)pyridin-2-yl)-N-(4-(3,3-dimethylureido)benzyl)-1-isopropyl-4,5,6,7-tetrahydro-1H-pyrazolo[4,3-c]pyridine-3-carboxamide (286)

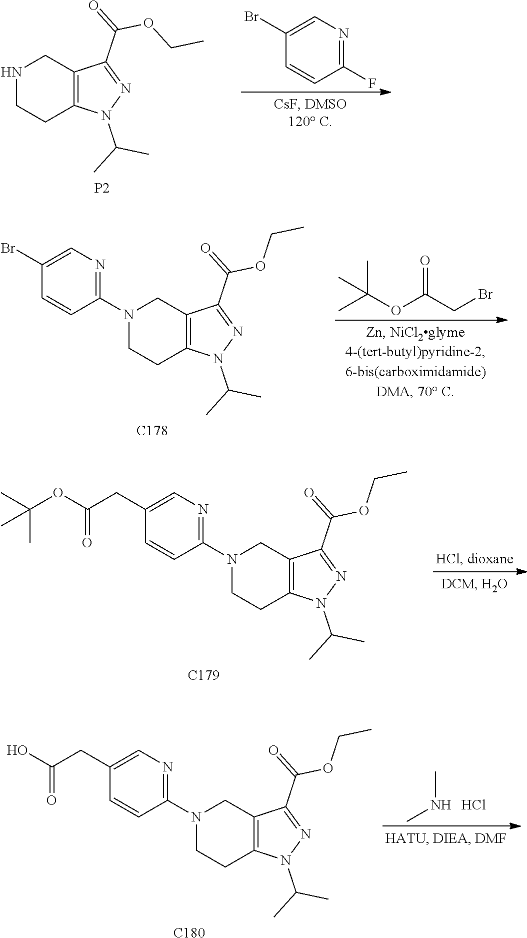

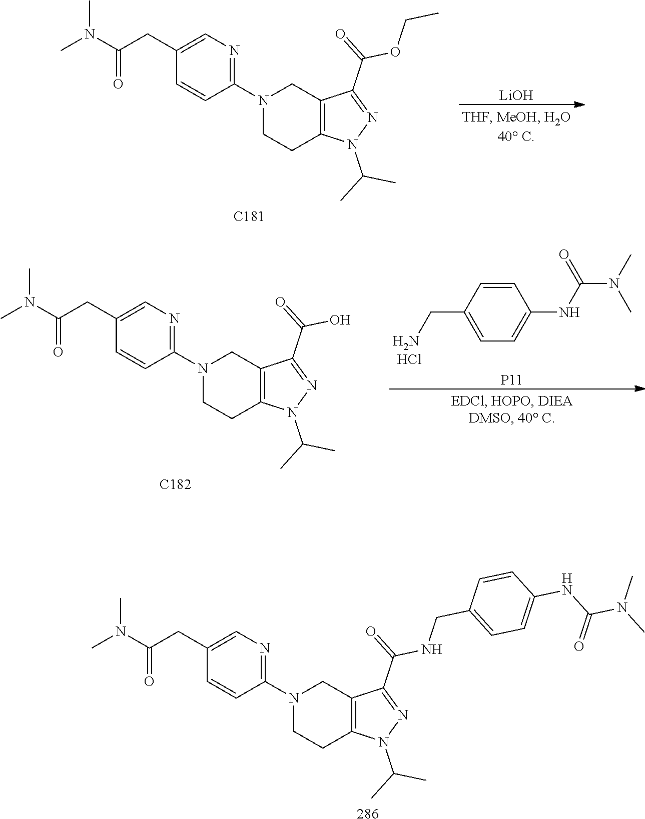

### Step 1. Preparation of ethyl 5-(5-bromopyridin-2-yl)-1-isopropyl-4,5,6,7-tetrahydro-1H-pyrazolo[4,3-c]pyridine-3-carboxylate (C178)

To a solution of P2 (4.10 g, 15.0 mmol) and CsF (5.69 g, 37.4 mmol) in DMSO (50 mL) was added 5-bromo-2-fluoropyridine (CAS: 766-11-0; 2.64 g, 15.0 mmol). The reaction mixture was stirred at 120° C. for 5 h. The solution was diluted with H₂O (40 mL) and extracted with EtOAc (3×50 mL). The organic layer was washed with brine (50 mL), dried with Na₂SO₄, and concentrated in vacuo. The residue was purified by column chromatography (silica gel, 0-25% EtOAc:PE) to provide C178 (3.20 g, 54.3% yield) as a yellow solid. ¹H NMR (400 MHz, (CD₃)₂SO) δ 8.18 (d, 1H), 7.70 (dd, 1H), 6.90 (d, 1H), 4.64 (s, 2H), 4.56-4.45 (m, 1H), 4.28 (q, 2H), 3.87 (t, 2H), 2.80 (t, 2H), 1.36 (d, 6H), 1.30 (t, 3H).

### Step 2. Preparation of ethyl 5-(5-(2-(tert-butoxy)-2-oxoethyl)pyridin-2-yl)-1-isopropyl-4,5,6,7-tetrahydro-1H-pyrazolo[4,3-c]pyridine-3-carboxylate (C179)

Under nitrogen gas, to C178 (0.58 g, 1.5 mmol) was added tert-butyl bromoacetate (CAS: 1211526-62-3; 0.29 g, 1.5 mmol), Zn (0.27 g, 3.7 mmol), NiCl₂·glyme (32 mg, 0.15 mmol), 4-(tert-butyl)pyridine-2,6-bis(carboximidamide) (CAS: 3003061-14-8; 43 mg, 0.15 mmol) and DMA (10 mL). The solution was degassed with nitrogen gas for 2 min and then stirred at 70° C. for 16 h. The reaction mixture was filtered, and the filtrate was concentrated in vacuo. The residue was diluted with EtOAc (20 mL) and then washed with H₂O (3×10 mL) followed by brine (2×10 mL). The organic layer was dried over Na₂SO₄, filtered, and concentrated in vacuo. The residue was purified by column chromatography (silica gel, 0-30% EtOAc:PE) to provide C179 (87 mg, 14% yield) as a colorless oil. (LC/MS) m/z (M+H)⁺=429.2.

### Step 3. Preparation of 2-(6-(3-(ethoxycarbonyl)-1-isopropyl-1,4,6,7-tetrahydro-5H-pyrazolo[4,3-c]pyridin-5-yl)pyridin-3-yl)acetic acid (C180)

To a solution of C179 (87 mg, 0.20 mmol) in DCM (3.0 mL), 2M HCl in dioxane (0.20 g, 6.0 mmol) and H₂O (0.1 mL) were added. The reaction mixture was stirred at room temperature for 2 h and then concentrated in vacuo to provide C180 (76 mg, crude) as a colorless oil. The oil was used directly in the next step without further purification.

### Step 4. Preparation of ethyl 5-(5-(2-(dimethylamino)-2-oxoethyl)pyridin-2-yl)-1-isopropyl-4,5,6,7-tetrahydro-1H-pyrazolo[4,3-c]pyridine-3-carboxylate (C181)

To a solution of C180 (78 mg, 0.21 mmol) in DMF (5.0 mL) was added HATU (0.13 g, 0.34 mmol), DIEA (0.15 g, 1.1 mmol), and dimethylamine hydrochloride (CAS: 506-59-2; 28 mg, 0.34 mmol). The reaction mixture was stirred at room temperature for 1 h, diluted with H₂O (4 mL), and extracted with EtOAc (3×5 mL). The organic layer was washed with brine (5 mL), dried over Na₂SO₄, and concentrated in vacuo. The residue was purified by column chromatography (silica gel, 0-80% EtOAc:PE) to provide C181 (0.12 g, 87% yield) as a yellow oil. (LC/MS) m/z (M+H)⁺=400.3.

### Step 5. Preparation of 5-(5-(2-(dimethylamino)-2-oxoethyl)pyridin-2-yl)-1-isopropyl-4,5,6,7-tetrahydro-1H-pyrazolo[4,3-c]pyridine-3-carboxylic acid (C182)

To a solution of C181 (0.12 g, 0.30 mmol) in THF (4.0 mL), MeOH (0.5 mL), and H₂O (1.0 mL) was added LiOH (22 mg, 0.90 mmol). The reaction mixture was then stirred at 40° C. for 3 h then was purified by reverse phase HPLC (C₁₈ 150 mm×30 mm×5 μm column, Mobile Phase A: H₂O (0.05% formic acid)/Mobile Phase B: ACN, 0 to 34% of Mobile Phase B over 9 min, 100% of Mobile Phase B hold 2 min, flow rate: 30 mL/min) and lyophilized to provide C182 (35 mg, 31% yield) as a white solid.

### Step 6. Preparation of 5-(5-(2-(dimethylamino)-2-oxoethyl)pyridin-2-yl)-N-(4-(3,3-dimethylureido)benzyl)-1-isopropyl-4,5,6,7-tetrahydro-1H-pyrazolo[4,3-c]pyridine-3-carboxamide (286)

To a solution of C182 (35 mg, 0.094 mmol) in DMSO (3.0 mL) was added DIEA (61 mg, 0.47 mmol), EDCl (43 mg, 0.23 mmol), and HOPO (31 mg, 0.28 mmol). The reaction mixture was stirred at 40° C. for 15 min, and then P11 (26 mg, 0.11 mmol) was added. The suspension was stirred at 40° C. for 12 h then purified by HPLC (C₁₈ 150 mm×40 mm×5 μm column, Mobile Phase A: H₂O (NH₄OH—NH₄HCO₃)/Mobile Phase B: ACN, 5 to 45% of Mobile Phase B over 9 min, 100% of Mobile Phase B hold 2 min, flow rate: 60 mL/min) to provide 286 (26 mg, 50% yield) as a white solid. (LC/MS) m/z (M+H)⁺=547.3. ¹H NMR (400 MHz, (CD₃)₂SO) δ 8.33-8.16 (m, 2H), 7.91 (s, 1H), 7.35 (d, 3H), 7.14 (d, 2H), 6.79 (d, 1H), 4.60 (s, 2H), 4.50-4.37 (m, 1H), 4.35-4.27 (m, 2H), 3.89-3.76 (m, 2H), 3.53-3.44 (m, 2H), 2.97 (s, 3H), 2.87 (s, 6H), 2.80-2.70 (m, 5H), 1.35 (d, 6H).

### Example 322

### rac-(R)—N-(4-(3,3-Dimethylureido)benzyl)-1-isopropyl-5-(5-(tetrahydrofuran-2-yl)pyridin-2-yl)-4,5,6,7-tetrahydro-1H-pyrazolo[4,3-c]pyridine-3-carboxamide (322)

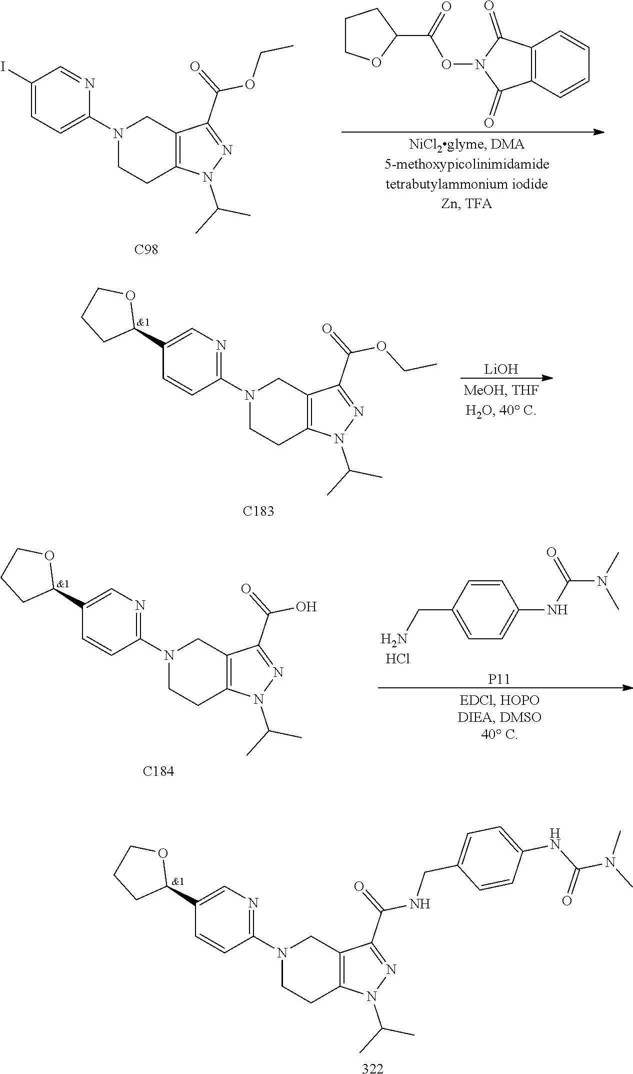

### Step 1. Preparation of rac-ethyl (R)-1-isopropyl-5-(5-(tetrahydrofuran-2-yl)pyridin-2-yl)-4,5,6,7-tetrahydro-1H-pyrazolo[4,3-c]pyridine-3-carboxylate (C183)

A reaction mixture of NiCl₂·glyme (59.9 mg, 0.273 mmol) and 5-methoxypicolinimidamide (41.2 mg, 0.273 mmol) in DMA (1.4 mL) was stirred at room temperature for 2 min before C98 (0.120 g, 0.273 mmol) and 1,3-dioxoisoindolin-2-yl tetrahydrofuran-2-carboxylate (CAS: 1872262-69-5; 0.107 g, 0.409 mmol) were added. The reaction mixture was stirred for 2 min then tetrabutylammonium iodide (CAS: 311-28-4; 0.101 g, 0.273 mmol), preactivated Zn (0.125 g, 1.91 mmol), and TFA (15.5 mg, 0.136 mmol) were added. The suspension was stirred at room temperature for 16 h and then diluted with H₂O (30 mL) and EtOAc (20 mL). The mixture was filtered through Celite. The filtrate was concentrated in vacuo and the residue was purified by reverse phase HPLC (C18 150 mm×40 mm×5 μm column, Mobile Phase A: H₂O (NH₄OH—NH₄HCO₃)/Mobile Phase B: ACN, 39 to 59% of Mobile Phase B over 9 min, 100% of Mobile Phase B hold 2 min, flow rate: 60 mL/min) to provide C183 (25.1 mg, 26.6% yield) as a gum. (LC/MS) m/z (M+H)⁺=385.2. ¹H NMR (400 MHz, CD₃OD) δ 8.09 (d, 1H), 7.59 (dd, 1H), 6.89 (d, 1H), 4.81-4.73 (m, 1H), 4.67 (s, 2H), 4.63-4.48 (m, 1H), 4.40 (q, 2H), 4.11-4.01 (m, 1H), 3.96 (t, 2H), 3.93-3.84 (m, 1H), 2.86 (t, 2H), 2.35-2.23 (m, 1H), 2.12-1.98 (m, 2H), 1.87-1.73 (m, 1H), 1.52-1.36 (m, 9H).

### Step 2. Preparation of rac-(R)-1-isopropyl-5-(5-(tetrahydrofuran-2-yl)pyridin-2-yl)-4,5,6,7-tetrahydro-1H-pyrazolo[4,3-c]pyridine-3-carboxylic acid (C184)

To a solution of C183 (23.7 mg, 0.616 mmol) in THF (1.0 mL), MeOH (0.5 mL), and H₂O (0.5 mL) was added LiOH (7.76 mg, 0.185 mmol). The reaction mixture was stirred at 40° C. for 3 h, and then 2M HCl was added to pH˜5. The acidic yellow solution was concentrated in vacuo then lyophilized to form C184 (22.0 mg, crude) as a light brown solid. The solid was used directly in the next step without further purification. (LC/MS) m/z (M+H)⁺=357.1

### Step 3. Preparation of rac-(R)—N-(4-(3,3-dimethylureido)benzyl)-1-isopropyl-5-(5-(tetrahydrofuran-2-yl)pyridin-2-yl)-4,5,6,7-tetrahydro-1H-pyrazolo[4,3-c]pyridine-3-carboxamide (322)

A reaction mixture of C184 (22.0 mg, 0.0617 mmol), HOPO (20.6 mg, 0.185 mmol), EDCl (28.4 mg, 0.148 mmol), and DIEA (39.9 mg, 0.309 mmol) in DMSO (1.0 mL) was stirred at 40° C. for 30 min. The suspension was added P11 (21.3 mg, 0.0926 mmol), and then the reaction mixture was stirred at 40° C. for 6 h. The yellow reaction mixture was purified with reverse phase HPLC (C18 150 mm×30 mm×5 μm column, Mobile Phase A: H₂O (NH₄OH—NH₄HCO₃)/Mobile Phase B: ACN, 16 to 56% of Mobile Phase B over 9 min, 100% of Mobile Phase B hold 2 min, flow rate: 30 mL/min) and lyophilized to provide 322 (18.4 mg, 55.9% yield) as a white solid. (LC/MS) m/z (M+H)⁺=532.2. ¹H NMR (400 MHz, CD₃OD) δ 8.10 (d, 1H), 7.61 (dd, 1H), 7.39-7.35 (m, 2H), 7.34-7.28 (m, 2H), 6.91 (d, 1H), 4.81-4.76 (m, 1H), 4.70 (s, 2H), 4.56-4.50 (m, 3H), 4.12-3.87 (m, 4H), 3.05-3.03 (m, 6H), 2.87 (t, 2H), 2.36-2.24 (m, 1H), 2.15-2.00 (m, 2H), 1.90-1.77 (m, 1H), 1.49 (d, 6H). The chiral purity was determined using SFC analytical method on Chiralcel OD-3 50 mm×4.6 mm×3 μm column; Mobile phase A: CO₂; Mobile phase B: EtOH with 0.2% of NH₃; isocratic: 40% of Mobile Phase B; flow rate: 4.0 mL/min; backpressure: 1500 psi; column temperature: 35° C. to show a mixture of peak 1 ((SFC-MS) m/z (M+H)⁺=532.16 at retention time: 0.734 min, 49.42% ee) and peak 2 ((SFC-MS) m/z (M+H)⁺=532.17 at retention time: 1.055 min, 50.58% ee).

### Example 338

### N-(4-(3,3-Dimethylureido)benzyl)-1-isopropyl-5-(5-(oxetan-3-yl)pyridin-2-yl)-4,5,6,7-tetrahydro-1H-pyrazolo[4,3-c]pyridine-3-carboxamide (338)

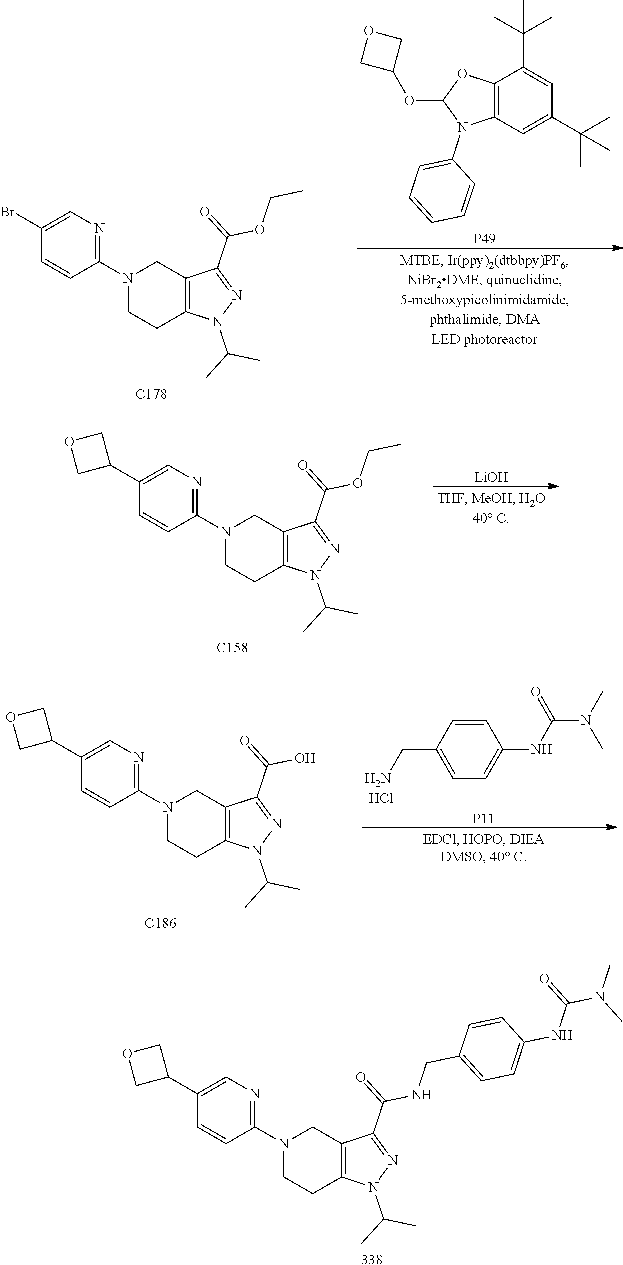

### Step 1. Preparation of ethyl 1-isopropyl-5-(5-(oxetan-3-yl)pyridin-2-yl)-4,5,6,7-tetrahydro-1H-pyrazolo[4,3-c]pyridine-3-carboxylate (C185)

In a glovebox, to C178 (0.100 g, 0.254 mmol) was added Ir(ppy)₂(dtbbpy)PF₆ (CAS: 676525-77-2; 3.49 mg, 0.00381 mmol), NiBr₂·DME (7.85 mg, 0.0254 mmol), 5-methoxypicolinimidamide (CAS: 1179532-65-0; 3.84 mg, 0.0254 mmol), phthalimide (9.35 mg, 0.0636 mmol), quinuclidine (49.5 mg, 0.445 mmol), and DMA (3 mL). A solution of P49 (194 mg, 0.509 mmol) in MTBE (3 mL) was added to the suspension via a syringe with a filter. The reaction vessel was sealed and irradiated with a photoreactor (maximum fan speed; stirred speed: 1500 rpm; 100% light intensity LED) for 16 h. The reaction mixture was concentrated in vacuo, then basified with aqueous NH₄OH and diluted with MeOH (1 mL). The solution was purified by reverse phase HPLC (C18 150 mm×40 mm×5 μm column, Mobile Phase A: H₂O (NH₄OH—NH₄HCO₃)/Mobile Phase B: ACN, 21 to 61% of Mobile Phase B over 9 min, 100% of Mobile Phase B hold 2 min, flow rate: 60 mL/min) to provide C185 (25.1 mg, 26.6% yield) as a gum. (LC/MS) m/z (M+H)⁺=371.3. ¹H NMR (400 MHz, CD₃OD) δ 8.05 (d, 1H), 7.78 (dd, 1H), 6.93 (d, 1H), 5.03 (dd, 2H), 4.71-4.62 (m, 4H), 4.60-4.46 (m, 1H), 4.38 (q, 2H), 4.23-4.11 (m, 1H), 3.94 (t, 2H), 2.84 (t, 2H), 1.45 (d, 6H), 1.41 (t, 3H).

### Step 2. Preparation of 1-isopropyl-5-(5-(oxetan-3-yl)pyridin-2-yl)-4,5,6,7-tetrahydro-1H-pyrazolo[4,3-c]pyridine-3-carboxylic acid (C186)

To a solution of C185 (25.1 mg, 0.0678 mmol) in THF (1.0 mL), MeOH (0.5 mL), and H₂O (0.5 mL) was added LiOH (8.53 mg, 0.203 mmol). The reaction mixture was then stirred at 40° C. for 2.5 h and then concentrated in vacuo. The residue was diluted with H₂O (0.5 mL), and then the pH was adjusted with diluted formic acid in H₂O to pH=6. The resulting yellow solution was lyophilized under high vacuum to provide C186 (23.2 mg, crude) as a light brown solid. This solid was used directly in the next step without further purification. (LC/MS) m/z (M+H)⁺=343.2.

### Step 3. Preparation of N-(4-(3,3-dimethylureido)benzyl)-1-isopropyl-5-(5-(oxetan-3-yl)pyridin-2-yl)-4,5,6,7-tetrahydro-1H-pyrazolo[4,3-c]pyridine-3-carboxamide (338)

A reaction mixture of C186 (23.2 mg, 0.0678 mmol), HOPO (22.6 mg, 0.203 mmol), EDCl (31.2 mg, 0.163 mmol), and DIEA (43.8 mg, 0.339 mmol) in DMSO (2.0 mL) was stirred at 40° C. for 30 min. P11 (23.4 mg, 0.102 mmol) was added to the suspension, and then the reaction mixture was stirred at 40° C. for 8 h. The yellow reaction mixture was purified with reverse phase HPLC (C₁₈ 150 mm×30 mm×5 μm column, Mobile Phase A: H₂O (NH₄OH—NH₄HCO₃)/Mobile Phase B: ACN, 9 to 52% of Mobile Phase B over 9 min, 100% of Mobile Phase B hold 2 min, flow rate: 30 mL/min) and lyophilized to provide 338 (6.51 mg, 18.6% yield) as a white solid. (LC/MS) m/z (M+H)⁺=518.3. ¹H NMR (400 MHz, CD₃OD) δ 8.06 (d, 1H), 7.79 (dd, 1H), 7.36-7.31 (m, 2H), 7.30-7.25 (m, 2H), 6.94 (d, 1H), 5.08-5.01 (m, 2H), 4.72-4.65 (m, 4H), 4.55-4.47 (m, 3H), 4.23-4.14 (m, 1H), 3.97 (t, 2H), 3.00 (s, 6H), 2.84 (t, 2H), 1.45 (d, 6H).

### Example 395

### (S)—N-(4-Carbamoylbenzyl)-5-(5-(6,7-dihydro-5H-pyrrolo[3,4-b]pyridine-6-carbonyl)pyridin-2-yl)-1-isopropyl-7-methyl-4,5,6,7-tetrahydro-1H-pyrazolo[4,3-c]pyridine-3-carboxamide (395)

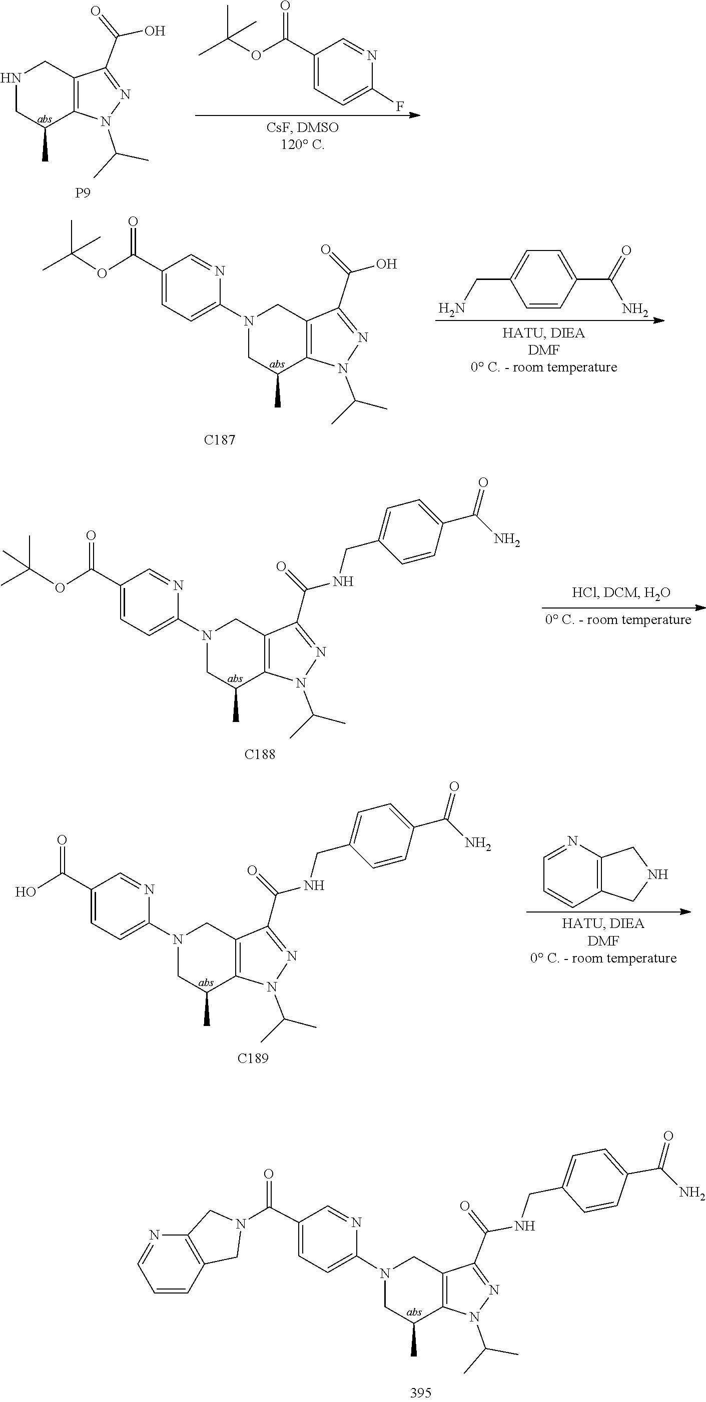

### Step 1. Preparation of (S)-5-(5-(tert-butoxycarbonyl)pyridin-2-yl)-1-isopropyl-7-methyl-4,5,6,7-tetrahydro-1H-pyrazolo[4,3-c]pyridine-3-carboxylic acid (C187)

To a reaction mixture of P9 (0.10 g, 0.45 mmol) and CsF (0.17 g, 1.1 mmol) in DMSO (4 mL) was added tert-butyl 6-fluoronicotinate (CAS: 676560-01-3, 88 mg, 0.45 mmol) and then stirred at 120° C. for 16 h. The reaction mixture was diluted with H₂O (5 mL) and then extracted with (1:10, MeOH:DCM, 3×2 mL). The combined organic layer was washed with brine (2×3 mL), dried with Na₂SO₄, filtered, and then concentrated in vacuo. The residue was purified by column chromatography (silica gel, 0-20% EtOAc:PE) to provide C187 (55 mg, 31% yield) as a yellow oil. (LCMS) m/z (M+H)⁺=401.2.

### Step 2. Preparation of tert-butyl (S)-6-(3-((4-carbamoylbenzyl)carbamoyl)-1-isopropyl-7-methyl-1,4,6,7-tetrahydro-5H-pyrazolo[4,3-c]pyridin-5-yl)nicotinate (C188)

At 0° C., to a solution of C187 (55 mg, 0.14 mmol) in DMF (2 mL) was added HATU (52 mg, 0.14 mmol) and then stirred for 30 min. Then DIEA (71 mg, 0.55 mmol) and P11 (26 mg, 0.14 mmol) were added. The reaction mixture was stirred at room temperature for 2 h and then poured into ice water (4 mL). The suspension was filtered, and the filter cake was collected to provide C188 (65 mg, 89% yield) as a solid. The solid was used directly in the next step without further purification. (LCMS) m/z (M+H)⁺=533.3.

### Step 3. Preparation of (S)-6-(3-((4-carbamoylbenzyl)carbamoyl)-1-isopropyl-7-methyl-1,4,6,7-tetrahydro-5H-pyrazolo[4,3-c]pyridin-5-yl)nicotinic acid hydrochloride (C189)

At 0° C., to a solution of C188 (0.14 g, 0.26 mmol) in DCM (2 mL) was added 2N HCl in dioxane (2 mL) and then stirred for 4 h. The reaction mixture was concentrated in vacuo to provide C189 (67 mg, crude) as a white solid. The solid was used directly in the next step without further purification. (LCMS) m/z (M+H)⁺=477.1.

### Step 4. Preparation of (S)—N-(4-carbamoylbenzyl)-5-(5-(6,7-dihydro-5H-pyrrolo[3,4-b]pyridine-6-carbonyl)pyridin-2-yl)-1-isopropyl-7-methyl-4,5,6,7-tetrahydro-1H-pyrazolo[4,3-c]pyridine-3-carboxamide (395)

To a solution of C189 (67 mg, 0.13 mmol) in DMF (2 mL) was added HATU (0.050 g, 0.13 mmol). The reaction mixture was stirred at 0° C. for 30 min, then DIEA (67 mg, 0.52 mmol) and 6,7-dihydro-5H-pyrrolo[3,4-b]pyridine (CAS: 147739-88-6; 16 mg, 0.13 mmol) were added.

The suspension was stirred for 2 h at room temperature, poured into ice water (8 mL), and extracted with EtOAc (3×5 mL). The combined organic layer was washed with Na₂CO₃ (2×5 mL) and brine (10 mL), dried with Na₂SO₄, and then concentrated in vacuo. The residue was dissolved in DMF then purified by reverse phase HPLC (Boston Prime C₁₈ 150 mm×30 mm×5 μm column; Mobile Phase A: H₂O (0.05% formic acid)/Mobile Phase B: ACN, 25 to 45% of Mobile Phase B over 10 min, held at 100% of Mobile Phase B for 2 min; flow rate: 35 mL/min) and lyophilized to provide 395 (19 mg, 25% yield) as a white solid. (LCMS) m/z (M+H)⁺=579.4. ¹H NMR (400 MHz, (CD₃)₂SO) δ 8.55 (t, 1H), 8.50-8.41 (m, 2H), 7.92-7.75 (m, 5H), 7.36 (d, 2H), 7.32-7.26 (m, 2H), 6.95-6.90 (m, 1H), 5.12 (d, 1H), 4.99-4.78 (m, 4H), 4.60-4.40 (m, 4H), 4.25 (d, 1H), 3.28-3.20 (m, 2H), 1.47-1.38 (m, 6H), 1.20 (d, 3H). The chiral purity was determined using SFC analytical method on Chiralpak AS-3 50×4.6 mm×3 μm column; Mobile phase A: CO₂/Mobile phase B: EtOH containing 0.2% NH₃; Isocratic: 40% of Mobile phase B; backpressure: 1500 psi; flow rate: 3.0 mL/min; column temperature: 35° C. to provide Example 395: peak 1 ((SFC-MS) m/z (M+H)⁺=579.19 at retention time: 1.586 minutes, 98% ee)

### Example 469

### rac-(R)—N-(4-(3,3-Dimethylureido)benzyl)-1-isopropyl-5-(5-(2-oxopyrrolidin-3-yl)pyridin-2-yl)-4,5,6,7-tetrahydro-1H-pyrazolo[4,3-c]pyridine-3-carboxamide (469)

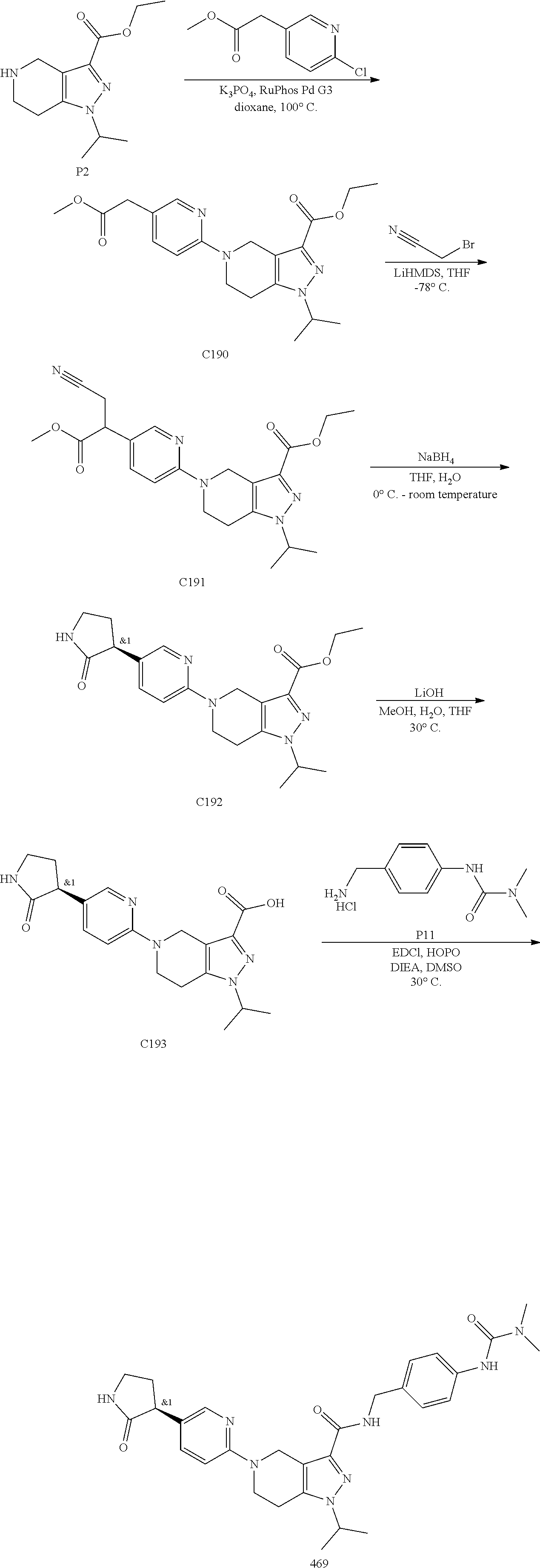

### Step 1. Preparation of ethyl 1-isopropyl-5-(5-(2-methoxy-2-oxoethyl)pyridin-2-yl)-4,5,6,7-tetrahydro-1H-pyrazolo[4,3-c]pyridine-3-carboxylate (C190)

A reaction mixture of methyl 2-(6-chloropyridin-3-yl)acetate (CAS: 717106-69-9; 1.40 g, 7.54 mmol), P2 (2.17 g, 7.92 mmol), K₃PO₄ (4.80 g, 22.6 mmol) and RuPhos Pd G3 (0.631 g, 0.754 mmol) in dioxane (50 mL) was degassed with nitrogen gas for 2 min. The reaction mixture was stirred at 100° C. for 24 h and then filtered. The filter cake was washed with EtOAc. The filtrate was purified by column chromatography (silica gel, 0-62% EtOAc:PE) to give C190 (2.22 g, 76.2% yield) as a yellow solid. ¹H NMR (400 MHz, CDCl₃) δ 8.07 (d, 1H), 7.45 (dd, 1H), 6.72 (d, 1H), 4.62 (s, 2H), 4.54-4.38 (m, 3H), 4.08-4.03 (m, 2H), 3.68 (s, 3H), 3.50 (s, 2H), 2.81 (t, 2H), 1.52 (d, 6H), 1.42 (t, 3H).

### Step 2. Preparation of ethyl 5-(5-(3-cyano-1-methoxy-1-oxopropan-2-yl)pyridin-2-yl)-1-isopropyl-4,5,6,7-tetrahydro-1H-pyrazolo[4,3-c]pyridine-3-carboxylate (C191)

At −78° C. under nitrogen gas, to a solution of C190 (1.72 g, 4.45 mmol) in THF (22 mL) was added LiHMDS (1.12 g, 6.68 mmol). The reaction mixture was stirred at −78° C. for 30 min under nitrogen gas, and then 2-bromoacetonitrile (CAS: 590-17-0; 0.667 g, 5.56 mmol) was added dropwise. The suspension was stirred at −78° C. for 2 h and quenched with NH₄Cl (20 mL). The reaction mixture was extracted with EtOAc (3×). The combined organic phase was washed with brine and then dried with Na₂SO₄ and filtered. The filtrate was concentrated in vacuo then purified by column chromatography (silica gel, 0-50% EtOAc:PE) to provide C191 (1.70 g, 69.6% yield) as a yellow gum. (LCMS) m/z (M+H)⁺=426.2. ¹H NMR (400 MHz, CDCl₃) δ 8.10 (d, 1H), 7.43 (dd, 1H), 6.74 (d, 1H), 4.65 (s, 2H), 4.55-4.38 (m, 3H), 4.17-4.05 (m, 2H), 3.89-3.80 (m, 1H), 3.72 (s, 3H), 2.98 (dd, 1H), 2.86-2.74 (m, 3H), 1.52 (d, 6H), 1.42 (t, 3H).

### Step 3. Preparation of rac-ethyl (R)-1-isopropyl-5-(5-(2-oxopyrrolidin-3-yl)pyridin-2-yl)-4,5,6,7-tetrahydro-1H-pyrazolo[4,3-c]pyridine-3-carboxylate (C192)

The reaction was conducted in two batches combined for the work-up and purification. At 0° C., under nitrogen gas, to a solution of C191 (1.50 g, 3.52 mmol) in THF (20 mL) and H₂O (3 mL), NaBH₄ (0.534 g, 14.1 mmol) was slowly added to form the first batch. The reaction mixture of the first batch was stirred for 3 h at room temperature and then quenched with saturated NH₄Cl (20 mL) and filtered through Celite.

A second batch of the same reaction was conducted with C191 (0.200 g, 0.470 mmol). The two batches were combined and extracted with EtOAc (3×50 mL). The combined organic layer was washed with brine (10 mL), dried over Na₂SO₄, filtered, and concentrated in vacuo. The residue was purified by column chromatography (silica gel, 0-65% EtOAc:PE) to provide C192 (0.920 g, 58.0% yield) as a white solid. (LCMS) m/z (M+H)⁺=398.2. ¹H NMR (400 MHz, CDCl₃) δ 8.10 (d, 1H), 7.45 (dd, 1H), 6.75 (d, 1H), 4.64 (s, 2H), 4.54-4.38 (m, 3H), 4.07 (t, 2H), 3.92-3.80 (m, 2H), 3.16-3.05 (m, 1H), 2.87-2.77 (m, 3H), 2.69 (dd, 1H), 1.70-1.66 (m, 1H), 1.52 (d, 6H), 1.43 (t, 3H).

### Step 4. Preparation of rac-(R)-1-isopropyl-5-(5-(2-oxopyrrolidin-3-yl)pyridin-2-yl)-4,5,6,7-tetrahydro-1H-pyrazolo[4,3-c]pyridine-3-carboxylic acid (C193)

The same reaction was conducted in two batches and combined for the work-up. To a solution of C192 (0.13 g, 0.33 mmol) in THF (20.0 mL), H₂O (3.0 mL), and MeOH (0.5 mL) was added LiOH (69 mg, 1.6 mmol) to form the first batch. The reaction mixture of the first batch was stirred at 30° C. for 3 h, and then the second batch of the same reaction was conducted with C192 (0.020 g, 0.050 mmol). The two batches were combined then the pH was adjusted to pH˜3 with 2M aqueous HCl. The aqueous phase was concentrated in vacuo to provide C193 (0.14 g, crude) as a white solid. The solid was used directly in the next step without further purification. (LCMS) m/z (M+H)⁺=370.1. ¹H NMR (400 MHz, (CD₃)₂SO) δ 12.64 (br s, 1H), 7.98 (d, 1H), 7.46 (dd, 1H), 6.81 (d, 1H), 5.00 (t, 1H), 4.56 (s, 2H), 4.50-4.37 (m, 1H), 3.80 (t, 2H), 3.49-3.38 (m, 2H), 2.92-2.78 (m, 2H), 2.77-2.66 (m, 3H), 1.30 (d, 6H).

### Step 5. Preparation of rac-(R)—N-(4-(3,3-dimethylureido)benzyl)-1-isopropyl-5-(5-(2-oxopyrrolidin-3-yl)pyridin-2-yl)-4,5,6,7-tetrahydro-1H-pyrazolo[4,3-c]pyridine-3-carboxamide (469)

The same reaction was conducted in two batches and then combined for purification. To a reaction mixture of C193 (0.11 g, 0.30 mmol) in DMSO (3 mL) was added DIEA (0.38 g, 3.0 mmol), HOPO (99 mg, 0.89 mmol), and EDCl (0.14 g, 0.71 mmol) for the first batch. The suspension was stirred at 30° C. for 15 min, and then P11 (0.10 g, 0.45 mmol) was added to form the first batch. The reaction mixture of the first batch was stirred at 30° C. for 5 h.

The second batch of the same reaction was conducted with C193 (0.020 g, 0.054 mmol). The two batches were combined then purified reverse phase HPLC (C₁₈ 150 mm×40 mm×5 μm column, Mobile Phase A: H₂O (0.05% formic acid)/Mobile Phase B: ACN, 5 to 35% of Mobile Phase B over 9 min then held at 100% of Mobile Phase B for 2 min; flow rate=60 mL/min) and lyophilized to provide 469 (0.080 g, 41.7% yield) as a white solid. (LC/MS) m/z (M+H)⁺=545.3. ¹H NMR (400 MHz, CDCl₃) δ 8.07 (d, 1H), 7.40 (dd, 1H), 7.37-7.33 (m, 2H), 7.31-7.27 (m, 2H), 7.16 (t, 1H), 6.79 (d, 1H), 6.32 (s, 1H), 4.70 (s, 2H), 4.55 (d, 2H), 4.42-4.31 (m, 1H), 4.08 (t, 2H), 3.93-3.76 (m, 2H), 3.12-3.00 (m, 7H), 2.86-2.75 (m, 3H), 2.71-2.60 (m, 1H), 1.44 (d, 6H). The chiral purity was determined using SFC analytical method on Chiralpak AD-3 50×4.6 mm×3 μm column; Mobile phase A: CO₂/Mobile phase B: EtOH containing 0.2% NH₃; Isocratic: 40% of Mobile phase B; backpressure: 1500 psi; flow rate: 4.0 mL/min; column temperature: 35° C. to show a mixture of peak 1 ((SFC-MS) m/z (M+H)⁺=545.55 at retention time: 1.006 minutes, 49.54% ee) and peak 2 ((SFC-MS) m/z (M+H)⁺=545.55 at retention time: 1.470 min, 50.46% ee).

### Example 485

### (S)-5-(4-(Dimethylcarbamoyl)phenyl)-1-isopropyl-7-methyl-N-(4-(4-(piperidin-4-yloxy)piperidine-1-carboxamido)benzyl)-4,5,6,7-tetrahydro-1H-pyrazolo[4,3-c]pyridine-3-carboxamide hydrochloride (485)

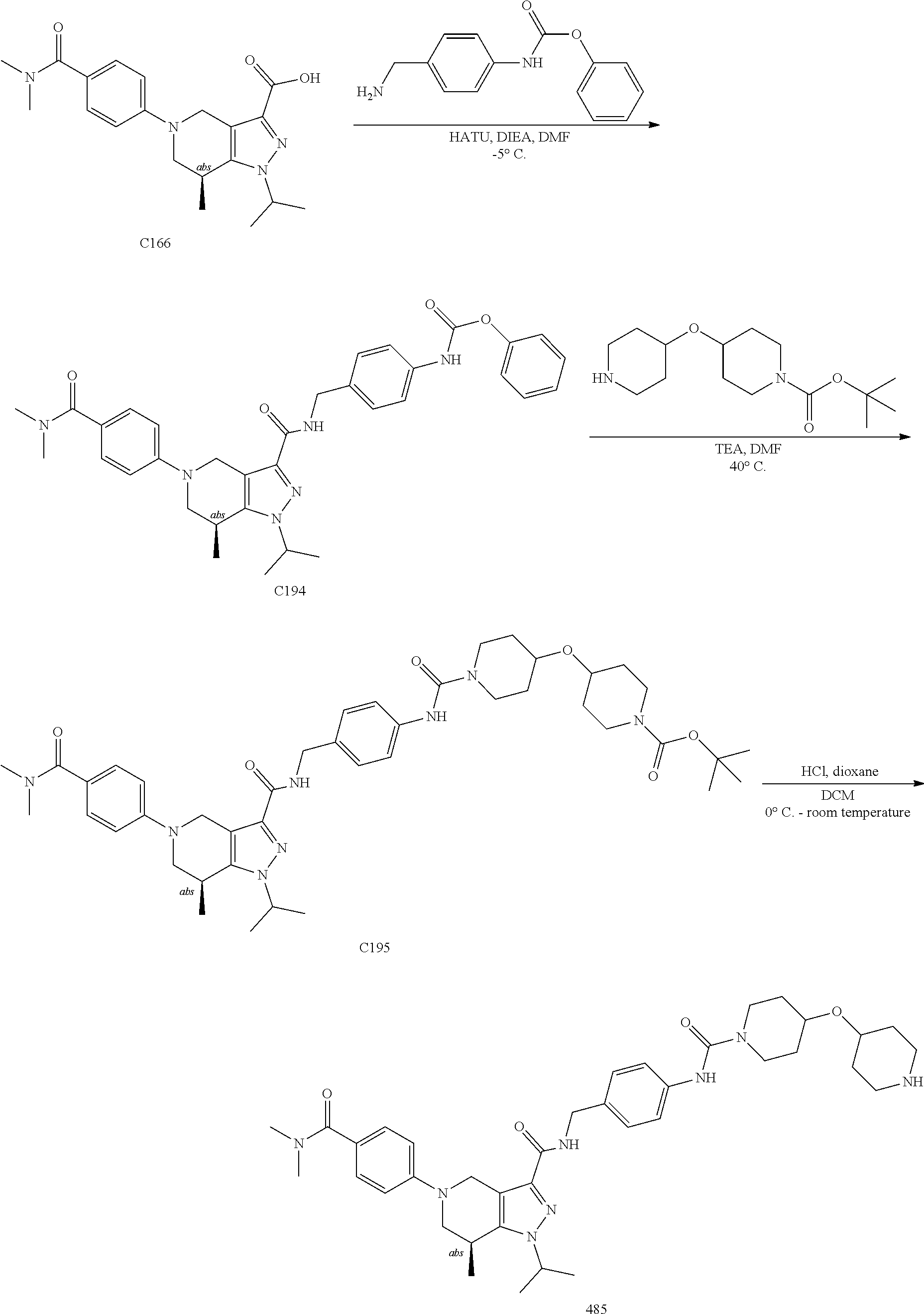

### Step 1. Preparation of phenyl (S)-(4-((5-(4-(dimethylcarbamoyl)phenyl)-1-isopropyl-7-methyl-4,5,6,7-tetrahydro-1H-pyrazolo[4,3-c]pyridine-3-carboxamido)methyl)phenyl)carbamate (C194)

At −5° C., to a reaction mixture of C166 (1.20 g, 3.24 mmol) and phenyl (4-(aminomethyl)phenyl)carbamate (CAS: 1019455-66-3; 0.957 g, 3.43 mmol) in DMF (16 mL) was added HATU (1.29 g, 3.40 mmol). The reaction mixture was stirred at −5° C. for 10 min, and then DIEA (1.67 g, 13.0 mmol) was added. The suspension was stirred at −5° C. for 1 h then was poured into ice water and filtered. The filter cake was washed with H₂O (3×15 mL) and lyophilized to provide C194 (1.73 g, 90.0% yield) as a white solid. (LC/MS) m/z (M+H)⁺=595.8. ¹H NMR (400 MHz, (CD₃)₂SO) δ 10.20-10.15 (m, 1H), 8.45-8.37 (m, 1H), 7.47-7.37 (m, 4H), 7.36-7.17 (m, 7H), 6.96 (d, 2H), 4.58-4.50 (m, 2H), 4.37 (d, 2H), 4.10 (d, 1H), 3.85-3.77 (m, 1H), 3.22-3.17 (m, 2H), 2.95 (s, 6H), 1.42 (dd, 6H), 1.28 (d, 3H).

### Step 2. Preparation of tert-butyl (S)-4-((1-((4-((5-(4-(dimethylcarbamoyl)phenyl)-1-isopropyl-7-methyl-4,5,6,7-tetrahydro-1H-pyrazolo[4,3-c]pyridine-3-carboxamido)methyl)phenyl)carbamoyl)piperidin-4-yl)oxy)piperidine-1-carboxylate (C195)

To a solution of C194 (0.300 g, 0.504 mmol) in DMF (3 mL) were added TEA (0.204 g, 2.02 mmol) and tert-butyl 4-(piperidin-4-yloxy)piperidine-1-carboxylate (CAS: 845305-83-1; 0.143 g, 0.504 mmol). The reaction mixture was stirred at 40° C. for 2 h then poured into H₂O and filtered. The filter cake was washed with H₂O (3×15 mL) and lyophilized to provide C195 (0.360 g, 90.9% yield) as a white solid. (LC/MS) m/z (M+H)⁺=785.5.

### Step 3. Preparation of (S)-5-(4-(dimethylcarbamoyl)phenyl)-1-isopropyl-7-methyl-N-(4-(4-(piperidin-4-yloxy)piperidine-1-carboxamido)benzyl)-4,5,6,7-tetrahydro-1H-pyrazolo[4,3-c]pyridine-3-carboxamide hydrochloride (485)

At 0° C., to a solution of C195 (0.361 g, 0.460 mmol) in DCM (5 mL) was added 2M HCl in dioxane (15 mL). The reaction mixture was stirred at room temperature for 2 h then concentrated in vacuo. The residue was diluted with H₂O then purified by reverse phase HPLC (Boston Green ODS 150 mm×30 mm×5 μm column; Mobile phase: A: H₂O (0.05% aqueous HCl)/Mobile Phase B: ACN; 15-55% of Mobile Phase B over 9.0 min; flow rate: 30 mL/min) and lyophilized to provide 485 (18.7 mg, 37.3% yield) as a yellow solid. (LC/MS) m/z (M+H)⁺=685.5. ¹H NMR (400 MHz, (CD₃)₂SO) δ 8.70-8.50 (m, 2H), 8.42-8.34 (m, 1H), 8.24 (t, 1H), 7.31-7.19 (m, 4H), 7.12-7.05 (m, 2H), 6.89-6.83 (m, 2H), 4.50-4.38 (m, 2H), 4.31-4.19 (m, 2H), 4.00 (d, 1H), 3.76-3.59 (m, 4H), 3.58-3.50 (m, 1H), 3.21-2.93 (m, 6H), 2.92-2.80 (m, 8H), 1.86-1.77 (m, 2H), 1.74-1.66 (m, 2H), 1.60-1.48 (m, 2H), 1.37-1.25 (m, 7H), 1.18 (d, 3H).

### Example 498

### (S)-2-(3-((4-(3,3-Dimethylureido)benzyl)carbamoyl)-1-isopropyl-7-methyl-1,4,6,7-tetrahydro-5H-pyrazolo[4,3-c]pyridin-5-yl)pyrimidine-5-carboxylic acid (498)

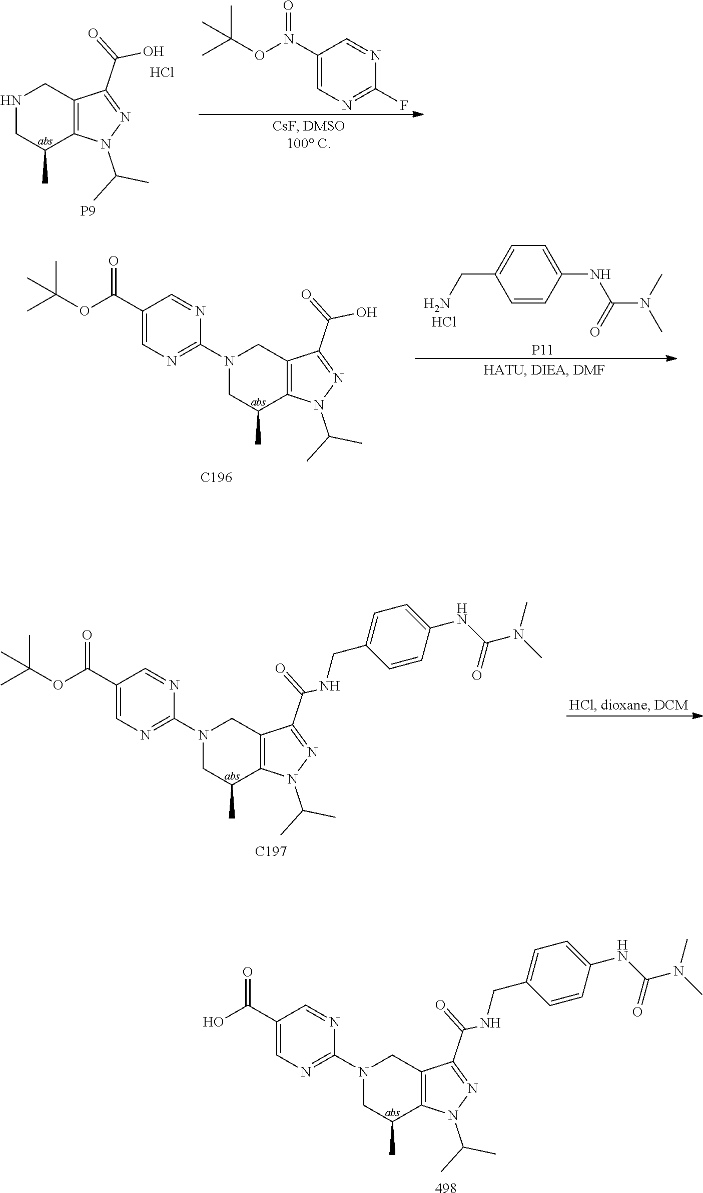

### Step 1. Preparation of (S)-5-(5-(tert-butoxycarbonyl)pyrimidin-2-yl)-1-isopropyl-7-methyl-4,5,6,7-tetrahydro-1H-pyrazolo[4,3-c]pyridine-3-carboxylic acid (C196)

To a solution of P9 (0.400 g, 1.54 mmol) and CsF (585 mg, 3.85 mmol) in DMSO (15 mL) was added tert-butyl 2-chloropyrimidine-5-carboxylate (CAS: 1864223-07-3; 331 mg, 1.54 mmol). The reaction mixture was stirred at 100° C. for 2 h and then diluted with H₂O (15 mL). The suspension was extracted with EtOAc (3×15 mL). The combined organic layer was washed with brine (3×40 mL) and then dried with Na₂SO₄ and concentrated in vacuo to provide C196 (0.610 g, crude) as a white solid. The solid was used directly in the next step without further purification. (LC/MS) m/z (M+H)⁺=402.2.

### Step 2. Preparation of tert-butyl (S)-2-(3-((4-(3,3-dimethylureido)benzyl)carbamoyl)-1-isopropyl-7-methyl-1,4,6,7-tetrahydro-5H-pyrazolo[4,3-c]pyridin-5-yl)pyrimidine-5-carboxylate (C197)

To a solution of C196 (0.610 g, 1.52 mmol) and P11 (349 mg, 1.52 mmol) in DMF (15 mL) was added HATU (607 mg, 1.60 mmol) and DIEA (786 mg, 6.08 mmol). The reaction mixture was stirred at room temperature for 30 min then was diluted with H₂O (20 mL) and extracted with EtOAc (3×20 mL). The combined organic layers were washed with brine (3×60 mL), dried with Na₂SO₄ and concentrated in vacuo. The resulting residue was purified by column chromatography (silica gel, 0-70% EtOAc:hexane) then lyophilized to provide C197 (0.780 g, 89.0% yield) as a solid. ¹H NMR (400 MHz, (CD₃)₂SO) δ 8.76 (s, 2H), 8.36 (t, 1H), 8.22 (s, 1H), 7.44-7.32 (m, 2H), 7.23-7.12 (m, 2H), 5.54 (d, 1H), 4.58-4.46 (m, 1H), 4.42-4.25 (m, 3H), 3.47-3.38 (m, 3H), 2.95-2.88 (m, 6H), 1.51 (s, 9H), 1.40 (dd, 6H), 1.10 (d, 3H).

### Step 3. Preparation of (S)-2-(3-((4-(3,3-dimethylureido)benzyl)carbamoyl)-1-isopropyl-7-methyl-1,4,6,7-tetrahydro-5H-pyrazolo[4,3-c]pyridin-5-yl)pyrimidine-5-carboxylic acid (498)

To a solution of C197 (0.780 g, 1.35 mmol) in DCM (3 mL) was added 2 M HCl in dioxane (20 mL). The reaction mixture was then stirred at room temperature for 5 h then concentrated in vacuo and lyophilized to provide 498 (0.701 g, crude) as a solid. The solid was used directly in the next step without further purification. (LC/MS) m/z (M+H)⁺=521.3. ¹H NMR (400 MHz, (CD₃)₂SO) δ 8.80 (s, 2H), 8.36 (t, 1H), 8.24 (s, 1H), 7.42-7.36 (m, 2H), 7.20-7.14 (m, 2H), 5.54 (d, 1H), 4.81 (d, 1H), 4.57-4.46 (m, 1H), 4.40-4.27 (m, 3H), 3.49-3.41 (m, 1H), 3.32-3.22 (m, 1H), 2.94-2.86 (m, 6H), 1.41 (dd, 6H), 1.13 (d, 3H). The chiral purity was determined using SFC analytical method on Chiralcel OJ-3 50 mm×4.6 mm×3 μm column; Mobile phase A: CO₂; Mobile phase B: EtOH with 0.2% of NH₃; gradient: 5-40% of Mobile Phase B over 1.5 min then held at 40% of Mobile Phase B for 1.0 min then 5% of Mobile Phase B for 0.5 min; flow rate: 3.0 mL/min; backpressure: 1500 psi; column temperature: 35° C. to provide Example 498: peak 1 ((SFC-MS) m/z (M+H)⁺=521.48 at retention time: 1.543 min, 100% ee).

### Example 515

### (S)-1-Isopropyl-7-methyl-5-(5-(1-methyl-1H-imidazol-2-yl)pyrimidin-2-yl)-N-(4-(3-methylureido)benzyl)-4,5,6,7-tetrahydro-1H-pyrazolo[4,3-c]pyridine-3-carboxamide (515)

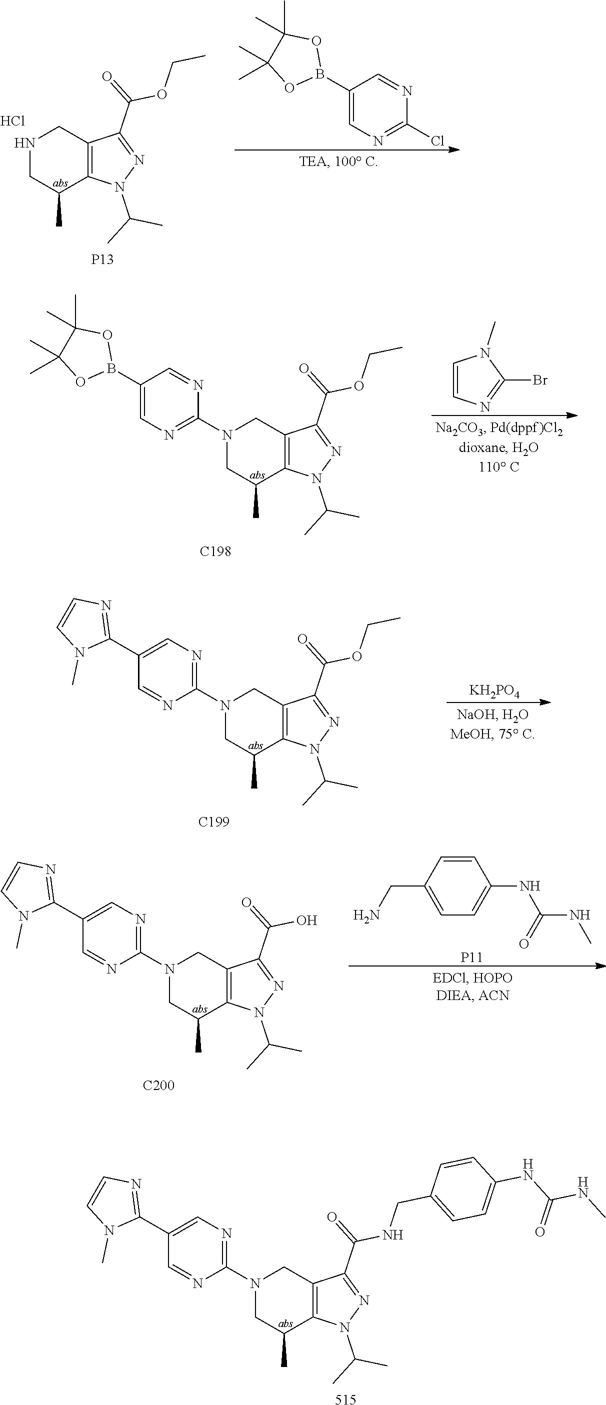

### Step 1. Preparation of ethyl (S)-1-isopropyl-7-methyl-5-(5-(4,4,5,5-tetramethyl-1,3,2-dioxaborolan-2-yl)pyrimidin-2-yl)-4,5,6,7-tetrahydro-1H-pyrazolo[4,3-c]pyridine-3-carboxylate (C198)

To a reaction mixture of P13 (0.598 g, 2.08 mmol) and TEA (0.463 g, 4.57 mmol) in dioxane (5.0 mL) was added 2-chloro-5-(4,4,5,5-tetramethyl-1,3,2-dioxaborolan-2-yl)pyrimidine (CAS: 1003845-08-6; 0.500 g, 2.08 mmol). The suspension was stirred at 100° C. for 2 h and then filtered. The filter cake was rinsed with dioxane (2.5 mL). The filtrate was diluted with saturated NaHCO₃ and extracted with EtOAc (2×25 mL). The combined organic layer was washed with brine (25 mL) then dried with Na₂SO₄ and concentrated in vacuo to give C198 (0.865 g, crude), which was used directly in the next step without further purification. (LC/MS) m/z (M+H)⁺=456.4.

### Step 2. Preparation of ethyl (S)-1-isopropyl-7-methyl-5-(5-(1-methyl-1H-imidazol-2-yl)pyrimidin-2-yl)-4,5,6,7-tetrahydro-1H-pyrazolo[4,3-c]pyridine-3-carboxylate (C199)

In a 2-neck round bottom flask was attaced a reflux condenser, to a reaction mixture of C198 (0.865 g, 1.90 mmol) in dioxane (17.6 mL) and H₂O (2.3 mL) was added Na₂CO₃ (0.604 g, 5.70 mmol) and 2-bromo-N-methylimidazole (CAS: 16681-59-7; 0.352 g, 2.18 mmol), reaction was purged with nitrogen gas for a few minutes. The reaction mixture was added Pd(dppf)Cl₂ (0.155 g, 0.190 mmol) and degassed with nitrogen gas. The suspension was stirred at 110° C. for ˜24 h and filtered over celite. The filter cake was washed with EtOAc (40 mL). The filtrate was concentrated in vacuo then purified by column chromatography (silica gel, 0-10% MeOH:DCM) then dried further over high vacuum to provide C199 (0.779 g, >95.0% yield). (LC/MS) m/z (M+H)⁺=410.3.

### Step 3. Preparation of (S)-1-isopropyl-7-methyl-5-(5-(1-methyl-1H-imidazol-2-yl)pyrimidin-2-yl)-4,5,6,7-tetrahydro-1H-pyrazolo[4,3-c]pyridine-3-carboxylic acid (C200)

To a stirred solution of C199 (0.779 g, 1.90 mmol) in MeOH (2.1 mL) was added 1M NaOH in H₂O (4.2 mL). The reaction mixture was stirred at 75° C. for 20 h before KH₂PO₄ (0.828 g, 6.09 mmol) was added and then cooled to room temperature. The suspension was concentrated in vacuo to remove the MeOH. Brine (10 mL) was added to the aqueous layer extracted with DCM (2×20 mL). The combined organic layer was dried with Na₂SO₄ and then concentrated in vacuo to provide C200 (0.419 g, 57.7% yield) as a brown oil. (LC/MS) m/z (M+H)⁺=382.3.

### Step 4. Preparation of (S)-1-isopropyl-7-methyl-5-(5-(1-methyl-1H-imidazol-2-yl)pyrimidin-2-yl)-N-(4-(3-methylureido)benzyl)-4,5,6,7-tetrahydro-1H-pyrazolo[4,3-c]pyridine-3-carboxamide (515)

To a reaction mixture of P11 (51.7 mg, 0.288 mmol), HOPO (35.0 mg, 0.315 mmol), EDCl (75.4 mg, 0.393 mmol), C200 (0.100 g, 0.262 mmol) in ACN (1.3 mL) was added DIEA (0.102 g, 0.786 mmol) then stirred at room temperature for ˜24 h. The suspension was filtered. The filter cake and filtrate were combined then concentrated in vacuo. The residue was purified reverse phase HPLC (Sunfire C₁₈ 100 mm×19 mm×5 μm column, Mobile Phase A: H₂O (0.05% TFA)/Mobile Phase B: ACN (0.05% TFA), 70 to 80% of Mobile Phase B over 10 min, flow rate=25 mL/min) and lyophilized to provide 515 (36.4 mg, 25.6% yield). (LC/MS) m/z (M+H)⁺=543.5. ¹H NMR (600 MHz, (CD₃)₂SO) δ 8.77 (s, 1H), 8.44 (s, 1H), 8.38 (t, 1H), 7.81-7.77 (m, 2H), 7.34-7.30 (m, 2H), 7.19-7.15 (m, 2H), 5.97-5.93 (m, 1H), 5.55 (d, 1H), 4.82 (d, 1H), 4.57-4.50 (m, 1H), 4.41-4.28 (m, 3H), 3.87-3.85 (m, 3H), 3.50-3.47 (m, 2H), 3.34-3.28 (m, 1H), 2.62 (d, 3H), 1.43 (dd, 6H), 1.16 (d, 3H).

### Example 523

### (4RS,7S)-5-(5-(Dimethylcarbamoyl)pyridin-2-yl)-N-(4-(3,3-dimethylureido)benzyl)-4-hydroxy-1-isopropyl-7-methyl-4,5,6,7-tetrahydro-1H-pyrazolo[4,3-c]pyridine-3-carboxamide (523)

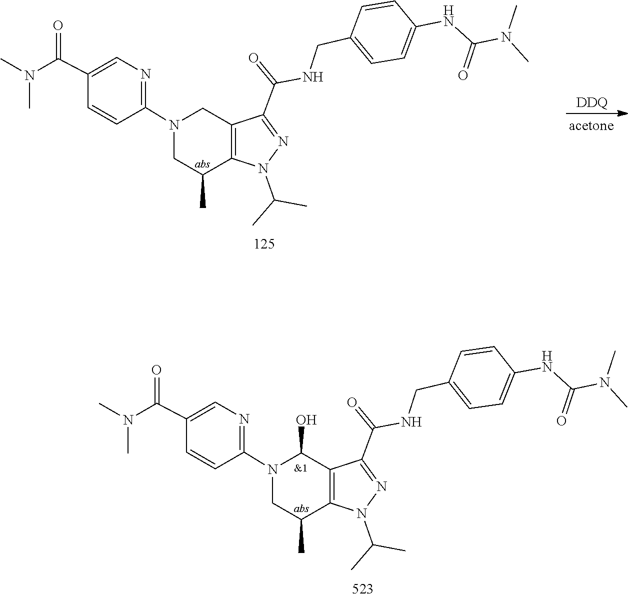

### Step 1. Preparation of (4RS,7S)-5-(5-(dimethylcarbamoyl)pyridin-2-yl)-N-(4-(3,3-dimethylureido)benzyl)-4-hydroxy-1-isopropyl-7-methyl-4,5,6,7-tetrahydro-1H-pyrazolo[4,3-c]pyridine-3-carboxamide (523)

To a reaction mixture of 125 (0.21 g, 0.38 mmol) in acetone (1.9 mL) was added DDQ (90 mg, 0.40 mmol). The suspension was stirred at room temperature under air for a few minutes and then diluted with saturated NaHCO₃ (40 mL). The mixture was extracted with DCM (40 mL), and then the organic layer was concentrated in vacuo. The residue was purified with SFC (Chiral Technologies OJ-H 250 mm×30 mm×5 μm column, Mobile Phase A: 80% CO₂/Mobile Phase B: 20% MeOH; flow rate: 80 mL/min; backpressure: 100 bar; column temperature: 40° C.) to provide 523 (0.090 g, 42% yield). ¹H NMR (600 MHz, (CD₃)₂SO) δ 8.61 (t, 1H), 8.28-8.22 (m, 2H), 7.71-7.63 (m, 1H), 7.44-7.38 (m, 2H), 7.23-7.16 (m, 2H), 7.10-7.01 (m, 1H), 6.62 (s, 1H), 5.98-5.95 (m, 1H), 4.69-4.64 (m, 1H), 4.58-4.51 (m, 1H), 4.46-4.34 (m, 2H), 3.49-3.43 (m, 1H), 3.22-3.19 (m, 1H), 3.03-2.95 (m, 6H), 2.92 (s, 6H), 1.50-1.34 (m, 6H), 1.14-1.05 (m, 3H).

### Example 524

### (4RS,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (524)

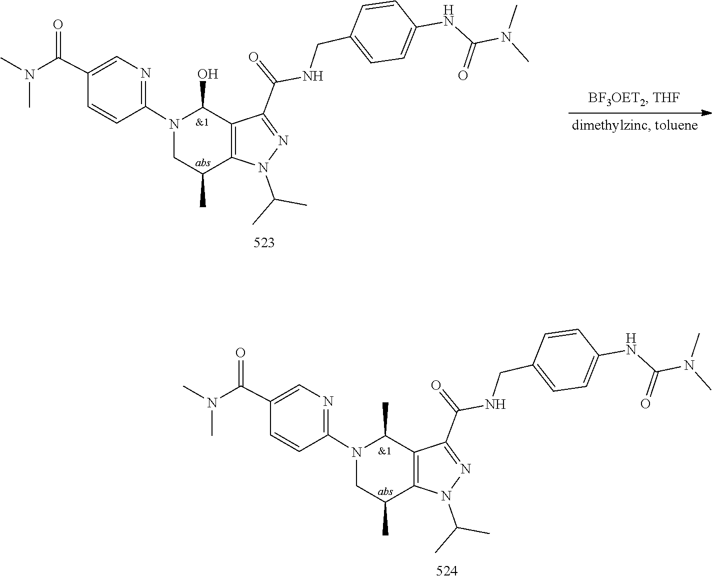

### Step 1. Preparation of (4RS,7S)-5-(5-(dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (524)

To a reaction mixture of 523 (0.100 g, 0.178 mmol) in THF (0.89 mL) was added BF₃OEt₂ (50.4 mg, 0.355 mmol). The suspension was stirred at room temperature for 15 min before 2M dimethylzinc in toluene (0.18 mL) was added. The reaction mixture was stirred at room temperature for ˜72 h then quenched with H₂O. The suspension was extracted with DCM (3×). The combined organic layer was concentrated in vacuo then purified by reverse phase HPLC (Sunfire C₁₈ 100 mm×19 mm×5 μm column, Mobile Phase A: H₂O (0.05% TFA)/Mobile Phase B: ACN (0.05% TFA), 65 to 85% of Mobile Phase B over 10 min, flow rate=25 mL/min) and lyophilized to provide 524 (0.100 g, 100% yield). LC/MS m/z (M+H)⁺=561.4. ¹H NMR (600 MHz, (CD₃)₂SO) δ 8.34 (t, 1H), 8.24-8.19 (m, 2H), 7.64 (d, 1H), 7.41-7.36 (m, 2H), 7.20-7.15 (m, 2H), 6.93 (d, 1H), 5.73 (s, 1H), 4.52-4.46 (m, 2H), 4.43-4.37 (m, 1H), 4.34-4.27 (m, 1H), 3.42 (d, 1H), 3.23-3.16 (m, 1H), 2.98 (s, 6H), 2.90 (s, 6H), 1.43-1.36 (m, 9H), 1.13 (d, 3H).

### Example 529

### rel-(4S*,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (C201) and (4R,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (529)

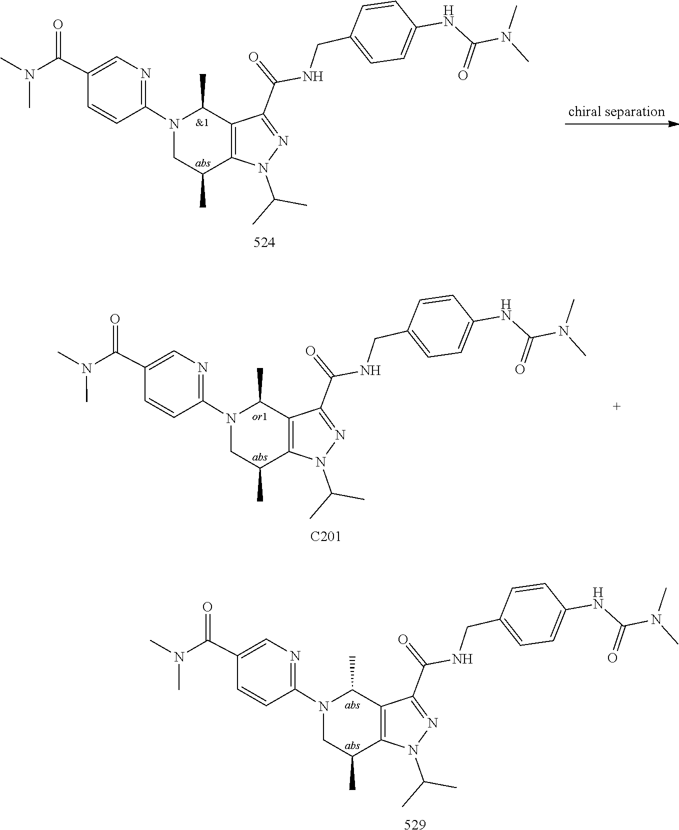

### Step 1. Preparation of rel-(4S*,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (C201) and (4R,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (529)

Example 524 (0.100 g, 0.178 mmol) was purified with SFC [Column: Phenomenex Lux Cellulose 1, 250 mm×21 mm×5 μm; Mobile phase: 30% MeOH (0.2% NH₄OH)/70% ACN; flow rate=75 mL/min, gradient time=20 min, backpressure=120 bar] to provide the minor first eluting isomer as Example C201 (0.30 mg, 3.0% yield) and the second eluting isomer as Example 529 (38 mg, 38% yield). LC/MS m/z (M+H)⁺=561.2. ¹H NMR (600 MHz, (CD₃)₂SO) δ 8.29 (t, 1H), 8.21-8.17 (m, 2H), 7.57 (dd, 1H), 7.37-7.31 (m, 2H), 7.18-7.10 (m, 2H), 6.83 (d, 1H), 5.70 (s, 1H), 4.50-4.40 (m, 2H), 4.39-4.30 (m, 1H), 4.29-4.20 (m, 1H), 3.31-3.27 (m, 1H), 3.18-3.08 (m, 1H), 2.94 (s, 6H), 2.86 (s, 6H), 1.39-1.27 (m, 9H), 1.08 (d, 3H). The chiral purity was determined using SFC analytical method on Lux Cellulose 1, 100 mm×4.6 mm×5 μm column; Mobile phase A: MeOH (0.2% NH₄OH); Mobile phase B: ACN; isocratic gradient: 60% of Mobile Phase B over 5 min; flow rate: 5.0 mL/min; backpressure: 120 bar to provide C201: peak 1 ((SFC-MS) m/z (M+H)⁺=561.2 at retention time: 3.02 min) and Example 529: peak 2 ((SFC-MS) m/z (M+H)⁺=561.2 at retention time: 3.28 min, 99% ee).

### Example 530

### (S)—N-(4-(3,3-Dimethylureido)benzyl)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxamide (530)

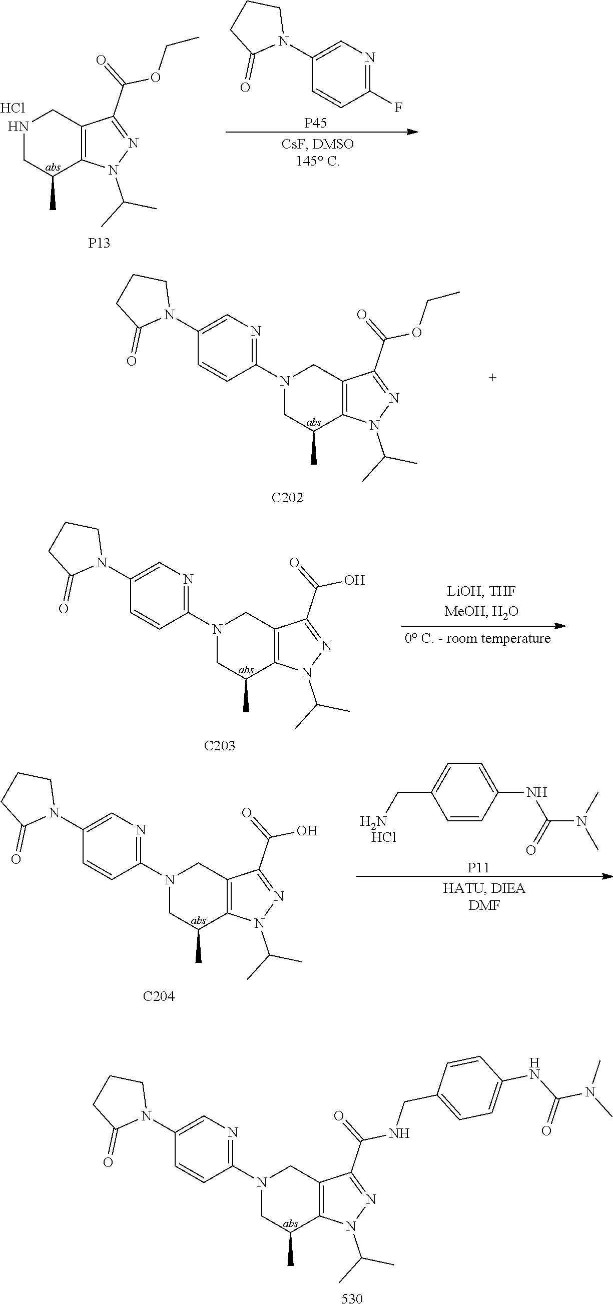

### Step 1. Preparation of ethyl (S)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxylate (C202) and (S)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxylic acid (C203)

To a solution of P45 (0.169 g, 0.938 mmol) and P13 (0.270 g, 0.938 mmol) in DMSO (3 mL) was added CsF (0.855 g, 5.63 mmol). The reaction mixture was stirred at 145° C. for 48 h then diluted with H₂O (10 mL) and acidified with aqueous 1M HCl to pH=4. The suspension was extracted with EtOAc (4×30 mL). The combined organic layer was washed with brine (20 mL) then dried with Na₂SO₄ and concentrated in vacuo to provide (61:22 according to LCMS) C203 (LC/MS) m/z (M+H)⁺=384.2: C202 (LC/MS) m/z (M+H)⁺=412.3 (0.290 g, crude) as a yellow gum. The gum was used directly in the next step without further purification.

### Step 2. Preparation of (S)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxylic acid (C204)

At 0° C., to the solution of a mixture of C202 and C203 (0.29 g, 0.17 mmol) in MeOH (2 mL) and THF (2 mL) were added LiOH (15 mg, 0.35 mmol) in H₂O (1 mL). The reaction mixture was stirred at room temperature for 16 h then concentrated in vacuo and diluted with H₂O (10 mL). The suspension was extracted with EtOAc (2×10 mL). The aqueous layer was acidified with 1M aqueous HCl to pH=3. The acidic aqueous phase was extracted with (1:10) (MeOH:DCM) (2×20 mL). The combined organic layer was washed with brine (2×20 mL) then dried with Na₂SO₄ and concentrated in vacuo to provide C204 (0.11 g, crude) as a yellow gum. The gum was used directly in the next step without further purification. (LC/MS) m/z (M+H)⁺=384.2

### Step 3. Preparation of (S)—N-(4-(3,3-dimethylureido)benzyl)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxamide (530)

To a solution of C204 (0.055 g, 0.14 mmol) and P11 (0.033 g, 0.14 mmol) in DMF (1 mL) was added DIEA (0.074 g, 0.57 mmol) and HATU (0.057 g, 0.15 mmol). The reaction was stirred at room temperature for 16 h then was filtered. The filtrate was purified by reverse phase HPLC (Boston Prime C₁₈ 150 mm×30 mm×5 μm column, Mobile Phase A: H₂O (0.05% TFA)/Mobile Phase B: ACN, 23 to 53% of Mobile Phase B over 12 min, 100% of Mobile Phase B hold 3 min, flow rate=35 mL/min) and lyophilized to provide 530 (19 mg, 24% yield) as a white solid. (LCMS) m/z (M+H)⁺=559.2. ¹H NMR (400 MHz, (CD₃)₂SO) δ 8.33 (t, 1H), 8.29 (d, 1H), 8.23 (s, 1H), 7.88 (dd, 1H), 7.43-7.35 (m, 2H), 7.21-7.15 (m, 2H), 6.90 (d, 1H), 4.95 (d, 1H), 4.59-4.48 (m, 1H), 4.40-4.28 (m, 3H), 4.17 (d, 1H), 3.78 (t, 2H), 3.32-3.18 (m, 2H), 2.91 (s, 6H), 2.44 (t, 2H), 2.12-2.00 (m, 2H), 1.47-1.38 (m, 6H), 1.21 (d, 3H). The chiral purity was determined using SFC analytical method on Chiralpak AS-3 50×4.6 mm×3 μm column; Mobile phase A: CO₂/Mobile phase B: EtOH containing 0.2% NH₃; Isocratic: 40% of Mobile phase B; backpressure: 1500 psi; flow rate: 3.0 mL/min; column temperature: 35° C. to provide Example 530: peak 1 ((SFC-MS) m/z (M+H)⁺=559.57 at retention time: 1.165 minutes, 99.53% ee)

### Example 537

### rac-(R)-7-(4-(Dimethylcarbamoyl)phenyl)-N-(4-(3,3-dimethylureido)benzyl)-3-isopropyl-5-methyl-5,6,7,8-tetrahydroimidazo[1,5-a]pyrazine-1-carboxamide (537)

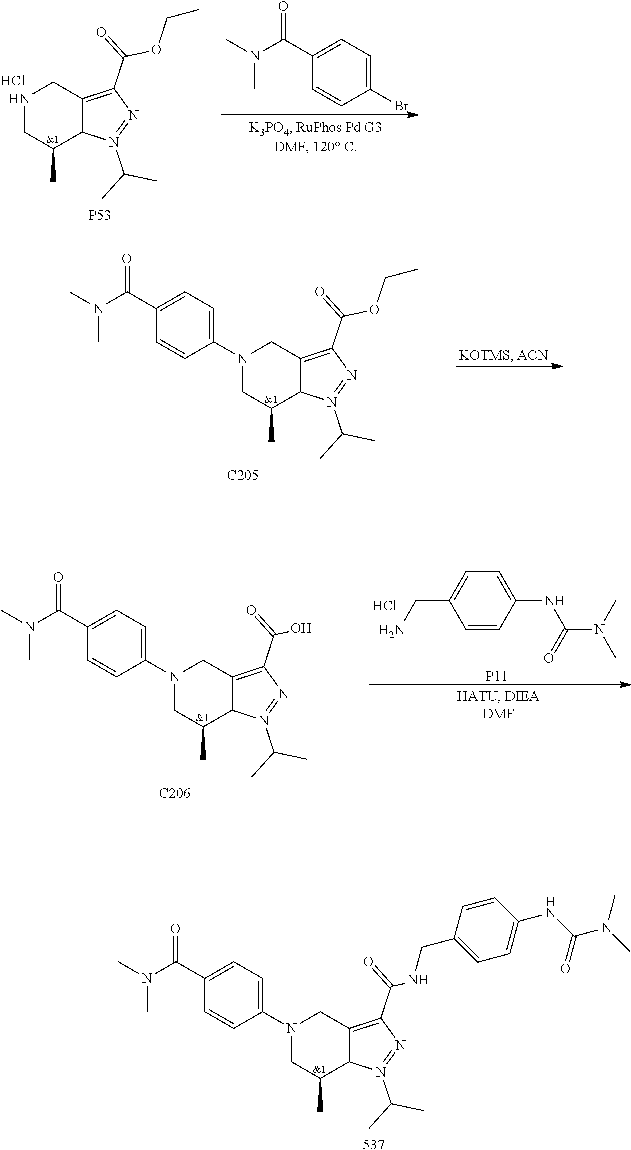

### Step 1. Preparation of rac-ethyl (R)-7-(4-(dimethylcarbamoyl)phenyl)-3-isopropyl-5-methyl-5,6,7,8-tetrahydroimidazo[1,5-a]pyrazine-1-carboxylate (C205)

To a reaction mixture of P53 (0.400 g, 1.39 mmol) in DMF (10 mL) was added 4-bromo-N,N-dimethylbenzamide (CAS: 18469-37-9; 0.396 g, 1.74 mmol), K₃PO₄ (0.885 g, 4.17 mmol) and RuPhos Pd G3 (0.119 g, 0.142 mmol). The suspension was stirred at 120° C. under nitrogen gas for 20 h. The reaction mixture was dissolved in MeOH and filtered. The filtrate was poured into H₂O (30 mL) and extracted with EtOAc (5×30 mL). The combined organic layers were washed with brine (3×100 mL), dried with Na₂SO₄, filtered and the filtrate was concentrated in vacuo to give a brown gum. The gum was purified by column chromatography (silica gel, 0-4% MeOH:DCM) to provide C205 (0.360 g, 65.0% yield) as yellow gum. (LC/MS) m/z (M+H)⁺=399.2. ¹H NMR (400 MHz, (CD₃)₂SO) δ 7.80-7.74 (m, 1H), 7.55-7.44 (m, 1H), 7.42-7.33 (m, 2H), 7.04-6.97 (m, 2H), 4.89 (d, 1H), 4.64-4.59 (m, 1H), 4.34 (d, 1H), 4.24 (q, 2H), 3.15-3.04 (m, 1H), 2.97 (s, 6H), 1.42 (d, 3H), 1.34-1.26 (m, 6H), 1.25-1.20 (m, 3H).

### Step 2. Preparation of rac-(R)-7-(4-(dimethylcarbamoyl)phenyl)-3-isopropyl-5-methyl-5,6,7,8-tetrahydroimidazo[1,5-a]pyrazine-1-carboxylic acid (C206)

Under nitrogen gas, to a reaction mixture of C205 (0.330 g, 0.828 mmol) in ACN (4 mL) was added KOTMS (0.319 g, 2.48 mmol). The suspension was stirred at room temperature for 2 h then acidified with 2M HCl in dioxane (0.7 mL) and concentrated in vacuo. The residue was diluted with brine (10 mL) and extracted with DCM (3×10 mL). The combined organic layers were dried over Na₂SO₄, filtered and the filtrate was concentrated in vacuo to provide C206 (0.280 g, 91.3% yield) as a yellow gum. (LC/MS) m/z (M+H)⁺=371.2.

### Step 3. Preparation of rac-(R)-7-(4-(dimethylcarbamoyl)phenyl)-N-(4-(3,3-dimethylureido)benzyl)-3-isopropyl-5-methyl-5,6,7,8-tetrahydroimidazo[1,5-a]pyrazine-1-carboxamide (537)

To a solution of C206 (0.280 g, 0.756 mmol) and P11 (0.139 g, 0.605 mmol) in DMF (4 mL) was added DIEA (0.391 g, 3.02 mmol) and HATU (0.244 g, 0.642 mmol). The reaction was stirred at room temperature for 1 h then diluted with DMF and ACN. The solution was purified by reverse phase HPLC (C18 150 mm×30 mm×5 μm column, Mobile Phase A: H₂O (0.05% formic acid)/Mobile Phase B: ACN (0.05% formic acid), 24 to 44% of Mobile Phase B over 10 min, 100% of Mobile Phase B hold 2.5 min, flow rate=30 mL/min) and lyophilized to provide 537 (0.160 g, 38.8% yield). (LCMS) m/z (M+H)⁺=546.3. ¹H NMR (400 MHz, (CD₃)₂SO) δ 8.22 (s, 1H), 8.04 (t, 1H), 7.42-7.35 (m, 4H), 7.21-7.14 (m, 2H), 7.03-6.96 (m, 2H), 4.93 (d, 1H), 4.64-4.58 (m, 1H), 4.43-4.31 (m, 3H), 4.02 (d, 1H), 3.41-3.38 (m, 1H), 3.15-3.04 (m, 1H), 2.97 (s, 6H), 2.91 (s, 6H), 1.42 (d, 3H), 1.33-1.21 (m, 6H). The chiral purity was determined using SFC analytical method on Chiralcel OD-3 50×4.6 mm×3 μm column; Mobile phase A: CO₂/Mobile phase B: EtOH containing 0.2% NH₃; Isocratic: 40% of Mobile phase B; backpressure: 1500 psi; flow rate: 4 mL/min; column temperature: 35° C. to show a mixture of peak 1 ((SFC-MS) m/z (M+H)⁺=546.25 at retention time: 0.609 minutes, 50.54% ee) and peak 2 ((SFC-MS) m/z (M+H)⁺=546.22 at retention time: 1.040 minutes, 49.46% ee).

### Example 538 and 539

### rel-(R or S)-7-(4-(Dimethylcarbamoyl)phenyl)-N-(4-(3,3-dimethylureido)benzyl)-3-isopropyl-5-methyl-5,6,7,8-tetrahydroimidazo[1,5-a]pyrazine-1-carboxamide (538 and 539)

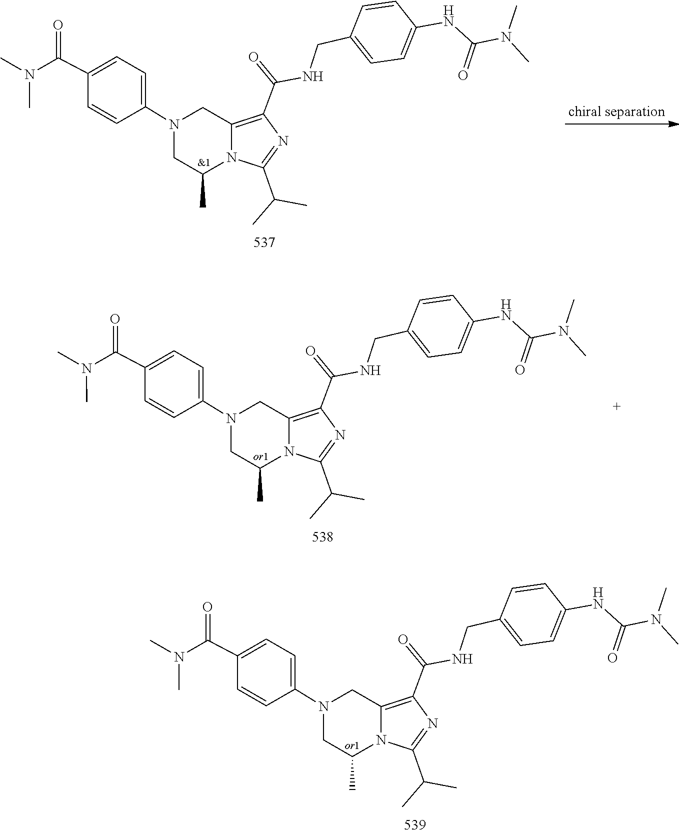

### Step 1. Separation of rel-(R or S)-7-(4-(dimethylcarbamoyl)phenyl)-N-(4-(3,3-dimethylureido)benzyl)-3-isopropyl-5-methyl-5,6,7,8-tetrahydroimidazo[1,5-a]pyrazine-1-carboxamide and rel-(R or S)-7-(4-(dimethylcarbamoyl)phenyl)-N-(4-(3,3-dimethylureido)benzyl)-3-isopropyl-5-methyl-5,6,7,8-tetrahydroimidazo[1,5-a]pyrazine-1-carboxamide (538 and 539)

Example 537 (0.130 g, 0.238 mmol) was purified with chiral SFC [Column: Daicel chiralcel OD C₁₈ 250 mm×30 mm×10 μm, Mobile phase: CO₂/EtOH (0.1% NH₄OH), 60% isocratic over 45 min, flow rate=80 mL/min] and lyophilized to provide the first eluting isomer as 539 (28.6 mg, 22.0% yield) and the second eluting isomer as 538 (27.3 mg, 21.0% yield). The chiral purity was determined using SFC analytical method on Chiralcel OD-3 50 mm×4.6 mm×3 μm column; Mobile phase A: CO₂; Mobile phase B: EtOH (0.2% NH₃); isocratic gradient: 40% of Mobile Phase B; flow rate: 4.0 mL/min; column temperature: 35° C., backpressure: 1500 psi to provide Example 539: peak 1 ((SFC-MS) m/z (M+H)⁺=546.24 at retention time: 0.607 min, 100% ee). ¹H NMR (400 MHz, (CD₃)₂SO) δ 8.19 (s, 1H), 8.02 (t, 1H), 7.38-7.28 (m, 4H), 7.18-7.07 (m, 2H), 7.00-6.91 (m, 2H), 4.88 (d, 1H), 4.60-4.52 (m, 1H), 4.38-4.26 (m, 3H), 3.97 (d, 1H), 3.42-3.38 (m, 1H), 3.11-3.00 (m, 1H), 2.92 (s, 6H), 2.86 (s, 6H), 1.36 (d, 3H), 1.27-1.17 (m, 6H) and Example 538: peak 2 ((SFC-MS) m/z (M+H)⁺=546.22 at retention time: 1.034 min, 100% ee). ¹H NMR (400 MHz, (CD₃)₂SO) δ 8.19 (s, 1H), 8.02 (t, 1H), 7.38-7.31 (m, 4H), 7.17-7.10 (m, 2H), 6.99-6.92 (m, 2H), 4.89 (d, 1H), 4.62-4.52 (m, 1H), 4.40-4.24 (m, 3H), 3.98 (d, 1H), 3.39-3.35 (m, 1H), 3.13-3.00 (m, 1H), 2.93 (s, 6H), 2.87 (s, 6H), 1.37 (d, 3H), 1.29-1.17 (m, 6H).

### Example 546

### rac-(R)-5-(5-(Dimethylcarbamoyl)pyridin-2-yl)-N-(4-(3,3-dimethylureido)benzyl)-1-isopropyl-7-(trifluoromethyl)-4,5,6,7-tetrahydro-1H-pyrazolo[4,3-c]pyridine-3-carboxamide (546)

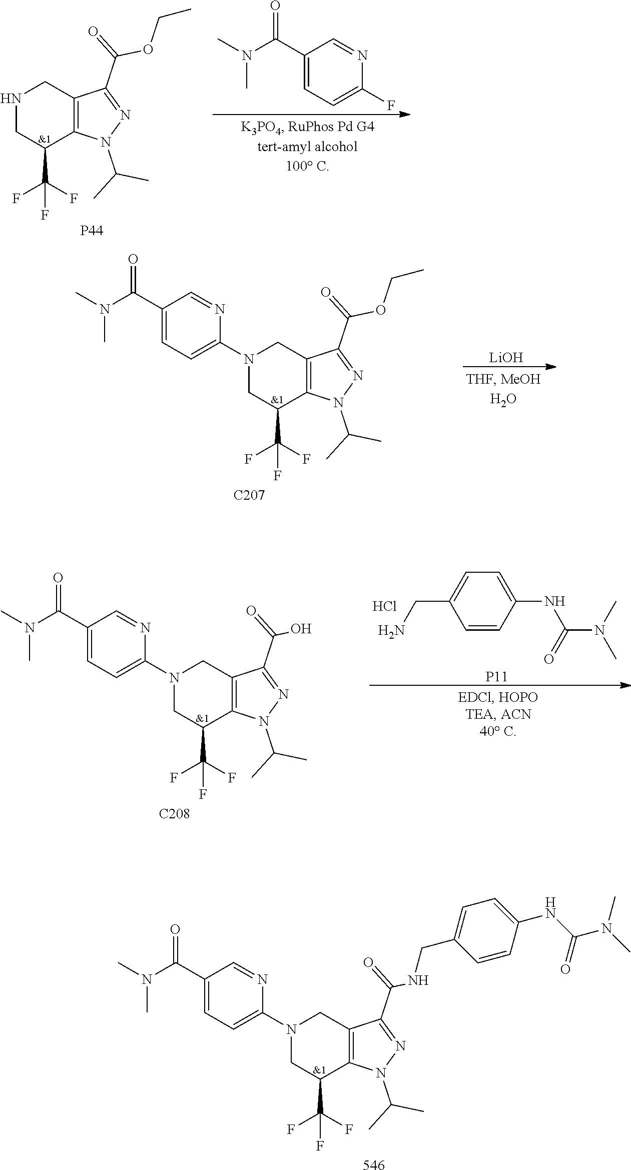

### Step 1. Preparation of rac-ethyl (R)-5-(5-(dimethylcarbamoyl)pyridin-2-yl)-1-isopropyl-7-(trifluoromethyl)-4,5,6,7-tetrahydro-1H-pyrazolo[4,3-c]pyridine-3-carboxylate (C207)

A reaction mixture of P44 (197 mg, 0.576 mmol), 6-chloro-N,N-dimethylnicotinamide (367 mg, 1.99 mmol) and K₃PO₄ (367 mg, 1.73 mmol) in tert-amyl alcohol (3.8 mL) was degassed with nitrogen gas for 5 min. To the suspension was added RuPhos Pd G4 (49.0 mg, 57.6 μmol). The reaction mixture was degassed with nitrogen gas for 5 min then stirred at 100° C. for 16 h. The suspension was diluted with EtOAc (10 mL) then filtered through Celite (1.5 g) prepacked on a plastic filter funnel. The filter cake was eluted with EtOAc. The filtrate was concentrated in vacuo. The residue was dissolved in EtOAc and heptane then concentrated in vacuo (2×). The solid was dissolved in DCM then purified by column chromatography (silica gel, 0-100% EtOAc:heptane) to provide C207 (145 mg, 55.4% yield) as a light-yellow solid. (LC/MS) m/z (M+H)⁺=454.6.

### Step 2. Preparation of rac-(R)-5-(5-(dimethylcarbamoyl)pyridin-2-yl)-1-isopropyl-7-(trifluoromethyl)-4,5,6,7-tetrahydro-1H-pyrazolo[4,3-c]pyridine-3-carboxylic acid

To a solution of C207 (0.140 g, 0.308 mmol) in THF (1.47 mL), MeOH (0.15 mL) and H₂O (1.47 mL) was added LiOH (39.6 mg, 1.65 mmol) at room temperature for 2 h. The reaction mixture was quenched with 1N HCl (1.4 mL) to pH=5-6 then stirred at room temperature for 1.5 h. The reaction mixture was acidified to pH=2-3 with 1N HCl (1.7 mL) then extracted with DCM (20 mL×1, 2×8 mL). The organic layers were combined then dried with Na₂SO₄, filtered, concentrated in vacuo and dried further under high vacuum to provide C208 (0.144 g, crude) as a light-yellow solid. (LC/MS) m/z (M+H)⁺=426.8. ¹H NMR (400 MHz, CDCl₃) δ 8.40 (d, 1H), 7.73 (dd, 1H), 6.78 (d, 1H), 4.66-4.55 (m, 1H), 4.40 (d, 1H), 3.79-3.66 (m, 3H), 3.31-3.23 (m, 1H), 3.11 (s, 6H), 1.60 (d, 3H), 1.52 (d, 3H).

### Step 3. Preparation of rac-(R)-5-(5-(dimethylcarbamoyl)pyridin-2-yl)-N-(4-(3,3-dimethylureido)benzyl)-1-isopropyl-7-(trifluoromethyl)-4,5,6,7-tetrahydro-1H-pyrazolo[4,3-c]pyridine-3-carboxamide (546)

A reaction mixture of C208 (0.060 g, 0.14 mmol), HOPO (19 mg, 0.17 mmol) and EDCl (41 mg, 0.21 mmol) in ACN (0.94 mL) was stirred at 40° C. for 30 min. After the stir, P11 (36 mg, 0.16 mmol) and TEA (43 mg, 0.42 mmol) were added. The reaction mixture was stirred at room temperature for 20 h. The residue was diluted with saturated NaHCO₃ (8 mL) and extracted with EtOAc (3×8 mL). The organic layers were combined then washed with brine then dried with Na₂SO₄, filtered and the filtrate was concentrated in vacuo. The residue was purified by column chromatography (silica gel, 0-55% MeOH:EtOAc) to give 546 (73 mg, 86% yield) as a white solid.

A portion of 546 (16 mg) was re-purified with achiral reverse phase HPLC (XBridge C₁₈ 100 mm×19 mm×5 μm column, Mobile Phase A: H₂O (0.05% TFA)/Mobile Phase B: ACN (0.05% TFA), 5 to 95% of Mobile Phase B over 9 min, 100% of Mobile Phase B hold 1 min, flow rate=25 mL/min) and lyophilized to provide 546 (14 mg, 85% yield). (LC/MS) m/z (M+H)⁺=601.9. ¹H NMR (600 MHz, (CD₃)₂SO) δ 8.57-8.52 (m, 1H), 8.29-8.27 (m, 1H), 8.26-8.22 (m, 1H), 7.72-7.67 (m, 1H), 7.43-7.38 (m, 2H), 7.23-7.15 (m, 2H), 6.87-6.82 (m, 1H), 4.72-4.65 (m, 1H), 4.56-4.50 (m, 1H), 4.41-4.33 (m, 2H), 4.31-4.26 (m, 1H), 4.13-4.07 (m, 1H), 3.22-3.15 (m, 2H), 2.99 (s, 6H), 2.93-2.90 (m, 6H), 1.48-1.40 (m, 6H).

### Example 547 and 548

### rel-(R or S)-5-(5-(Dimethylcarbamoyl)pyridin-2-yl)-N-(4-(3,3-dimethylureido)benzyl)-1-isopropyl-7-(trifluoromethyl)-4,5,6,7-tetrahydro-1H-pyrazolo[4,3-c]pyridine-3-carboxamide (547 and 548)

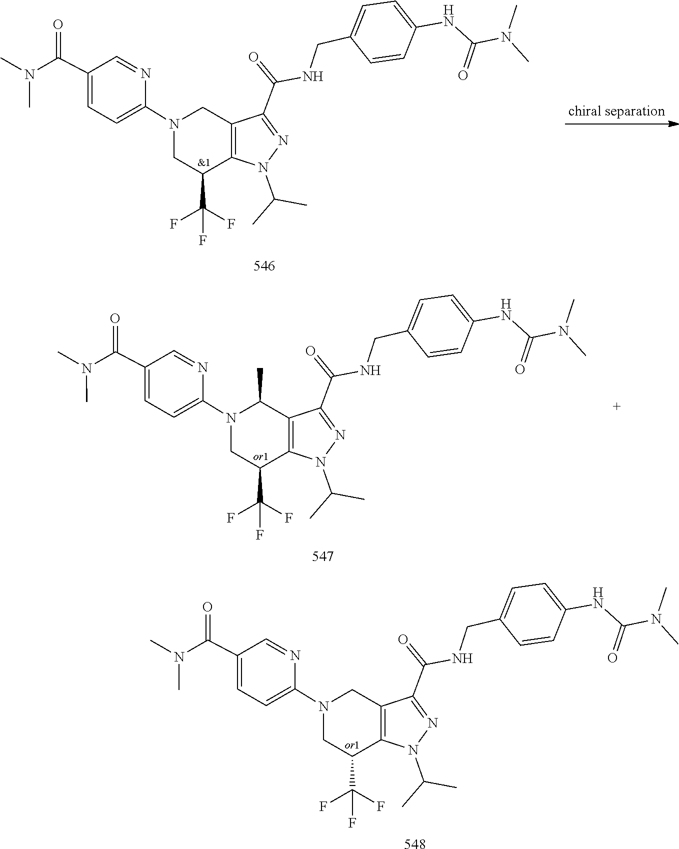

### Step 1. Separation of rel-(R or S)-5-(5-(dimethylcarbamoyl)pyridin-2-yl)-N-(4-(3,3-dimethylureido)benzyl)-1-isopropyl-7-(trifluoromethyl)-4,5,6,7-tetrahydro-1H-pyrazolo[4,3-c]pyridine-3-carboxamide (547 and 548)

546 (56 mg) was purified with chiral SFC [Column: Chiral Technologies OD-H C₁₈ 250 mm×21 mm×5 μm, Mobile phase: (3:2) CO₂/MeOH (0.2% NH₄OH), isocratic over 10 min, flow rate=75 mL/min; backpressure: 120 bar] and lyophilized to provide the first eluting isomer as 548 (22 mg, 38% yield) and the second eluting isomer as 547 (21 mg, 38% yield). The chiral purity was determined using SFC analytical method on Chiral Technologies OD-H 100 mm×4.6 mm×5 μm column; Mobile phase A: CO₂; Mobile phase B: MeOH (0.2% NH₄OH); isocratic gradient: 50% of Mobile Phase B; flow rate: 5.0 mL/min; backpressure: 120 bar to provide Example 548: peak 1 ((SFC-MS) m/z (M+H)⁺=601.3 at retention time: 1.99 min). and Example 547: peak 2 ((SFC-MS) m/z (M+H)⁺=601.3 at retention time: 3.94 min).

547: (LC/MS) m/z (M+H)⁺=601.3. ¹H NMR (600 MHz, (CD₃)₂SO) δ 8.55 (t, 1H), 8.29-8.27 (m, 1H), 8.24 (s, 1H), 7.69 (dd, 1H), 7.42-7.38 (m, 2H), 7.21-7.17 (m, 2H), 6.84 (d, 1H), 4.72-4.65 (m, 1H), 4.57-4.49 (m, 1H), 4.42-4.32 (m, 2H), 4.31-4.25 (m, 1H), 4.16-4.06 (m, 1H), 3.30-3.27 (m, 1H), 3.18-3.15 (m, 1H), 2.99 (s, 6H), 2.91 (s, 6H), 1.44 (dd, 6H).

### Example 561

### 5-(5-(Dimethylcarbamoyl)pyridin-2-yl)-N-(4-(3,3-dimethylureido) 2,3,5,6-d₄-benzyl)-1-isopropyl-4,5,6,7-tetrahydro-1H-pyrazolo[4,3-c]pyridine-3-carboxamide (561)

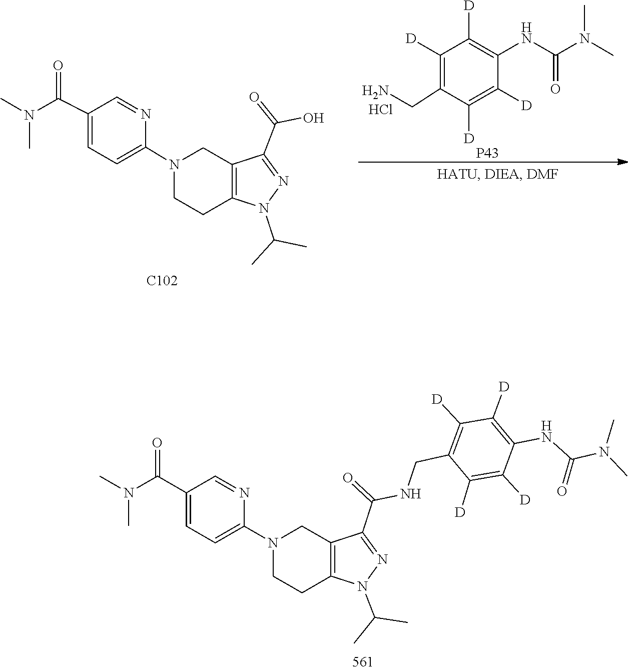

### Step 1. Preparation of 5-(5-(dimethylcarbamoyl)pyridin-2-yl)-N-(4-(3,3-dimethylureido) 2,3,5,6-d₄-benzyl)-1-isopropyl-4,5,6,7-tetrahydro-1H-pyrazolo[4,3-c]pyridine-3-carboxamide (561)

To a solution of C102 (0.940 g, 2.63 mmol) and P43 (1.05 g, 3.16 mmol) in DMF (10 mL) were added DIEA (1.36 g, 10.5 mmol) and HATU (1.10 g, 2.89 mmol). The reaction mixture was stirred at room temperature for 2 h then poured into H₂O (30 mL) and extracted with EtOAc:THF (2:1, 4×30 mL). The combined organic layer was washed with brine (3×50 mL) then dried with MgSO₄ and concentrated in vacuo. The residue was purified by column chromatography (silica gel, 5% MeOH:DCM). The resultant residue was slurried with MeOH (20 mL) at room temperature for 30 min then filtered. The filter cake was dried in vacuo then collected to provide 561 (0.509 g, 34.5% yield) as a white solid. (LC/MS) m/z (M+H)⁺=537.3.

¹H NMR (400 MHz, (CD₃)₂SO) δ 8.32 (t, 1H), 8.26-8.19 (m, 2H), 7.63 (dd, 1H), 6.88 (d, 1H), 4.75 (s, 2H), 4.55-4.43 (m, 1H), 4.35 (d, 2H), 3.95 (t, 2H), 2.97 (s, 6H), 2.91 (s, 6H), 2.82 (t, 2H), 1.39 (d, 6H).

### Example 567

### (S)-5-(4-(Dimethylcarbamoyl)phenyl)-1-isopropyl-7-methyl-N-(4-(4-methyl-1,4-diazepane-1-carboxamido)benzyl)-4,5,6,7-tetrahydro-1H-pyrazolo[4,3-c]pyridine-3-carboxamide (567)

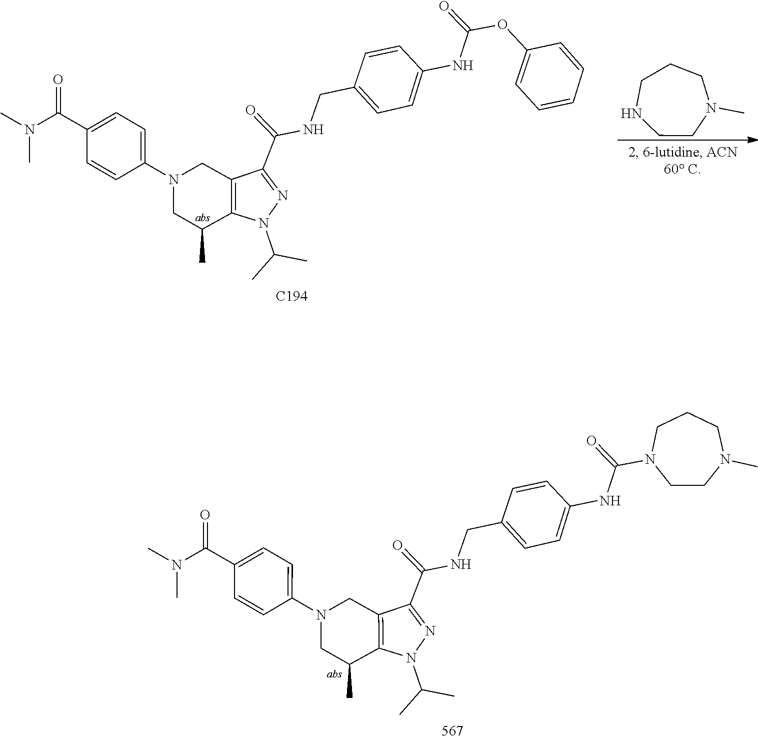

### Step 1. Preparation of (S)-5-(4-(dimethylcarbamoyl)phenyl)-1-isopropyl-7-methyl-N-(4-(4-methyl-1,4-diazepane-1-carboxamido)benzyl)-4,5,6,7-tetrahydro-1H-pyrazolo[4,3-c]pyridine-3-carboxamide (567)

To a reaction mixture of C194 (0.050 g, 0.079 mmol) and 1-methylhomopiperazine (CAS: 4318-37-0; 18 mg, 0.16 mmol) in ACN (0.8 mL) was added 2,6-lutidine (25 mg, 0.24 mmol). The suspension was stirred at 60° C. for 2 h then concentrated in vacuo. The residue was dissolved in DMSO then filtered and purified by reverse phase HPLC (Sunfire C18 100 mm×19 mm×5 μm column, Mobile Phase A: H₂O (0.05% TFA)/Mobile Phase B: ACN (0.05% TFA), 5 to 95% of Mobile Phase B over 10 min, flow rate=25 mL/min) and lyophilized to provide 567 (37 mg, 76% yield). (LC/MS) m/z (M+H)⁺=615.6.

### Examples 10-757

The compounds in Table 1 were prepared using similar chemistry as described above in the selected Examples 1-567 that are commercially available or that can be synthesized by literature methods to form the resultant compounds of the disclosure.

Lengthy table referenced here
US20250320213A1-20251016-T00001
Please refer to the end of the specification for access instructions.

### Chiral Purification Data:

Example 50\hat{ }: The chiral purity was determined using SFC analytical method on Chiralpak AD-3, 50 mm×4.6 mm×3 μm column; Mobile phase A: CO₂; Mobile phase B: EtOH with 0.2% of NH₃; isocratic 40% of Mobile Phase B; flow rate: 3.5 mL/min; column temperature: 35° C., backpressure: 1500 psi to provide Example 50 ((SFC-MS) m/z (M+H)⁺=519.37 at retention time: 2.047 min, 100% ee).

Example 51\hat{ }: A crude mixture was separated into its component isomers using SFC purification [Column: DAICEL CHIRALCEL OD 250 mm×30 mm×10 μm; Mobile phase: 45% CO₂/55% EtOH (0.1% NH₄OH); flow rate: 80 mL/min; gradient time=45 min]. The chiral purity was determined using SFC analytical method on Chiralcel OJ-3 50 mm×4.6 mm×3 μm column; Mobile phase A: CO₂; Mobile phase B: EtOH with 0.2% of NH₃; gradient: 5-40% of Mobile Phase B in 1.5 min and hold 40% for 1.0 min then 5% of Mobile Phase B for 0.5 min; flow rate: 3.0 mL/min; column temperature: 35° C., backpressure: 1500 psi to provide Example 51 ((SFC-MS) m/z (M+H)⁺=519.46 at retention time: 1.735 min, 100% ee).

Example 53 and 54\hat{ }: A mixture of Example 53 and Example 54 were separated into its component isomers using SFC [Column: DAICEL CHIRALCEL OD 250 mm×30 mm×10 μm; Mobile phase: 40% CO₂/60% isopropyl alcohol with 0.1% of NH₄OH; flow rate: 80 mL/min; gradient time: 45 min]. The chiral purity was determined using SFC analytical method on Chiralcel OD-3 50 mm×4.6 mm×3 μm column; Mobile phase A: CO₂; Mobile phase B: isopropanol with 0.05% of DEA; isocratic: 40% of Mobile Phase B; flow rate: 4.0 mL/min; backpressure: 1500 psi; column temperature: 35° C. to provide Example 54: peak 1 ((SFC-MS) m/z (M+H)⁺=519.30 at retention time: 2.019 min, 100% ee) and Example 53: peak 2 ((SFC-MS) m/z (M+H)⁺=519.28 at retention time: 3.607 min, 100% ee).

Example 58 and 78\hat{ }: Example 77 was separated into its component isomers using SFC [Column: DAICEL CHIRALCEL OD 250 mm×30 mm×10 μm; Mobile phase: 40% CO₂/60% MeOH (0.1% NH₄OH); flow rate: 80 mL/min; gradient time=45 min]. The chiral purity was determined using SFC analytical method on Chiralcel OD-3 50 mm×4.6 mm×3 μm column; Mobile phase A: CO₂; Mobile phase B: MeOH with 0.2% of NH₃; isocratic: 40% of Mobile Phase B; flow rate: 3.5 mL/min; backpressure: 1500 psi; column temperature: 35° C. to provide Example 58: peak 1 ((SFC-MS) m/z (M+H)⁺=533.41 at retention time: 0.778 min, 100% ee) and Example 78: peak 2 ((SFC-MS) m/z (M+H)⁺=533.41 at retention time: 1.392 min, 100% ee).

Example 63\hat{ }: A mixture was separated into its component isomers using SFC [Column: DAICEL CHIRALCEL OD 250 mm×30 mm×10 μm; Mobile phase: 40% CO₂/60% EtOH with 0.1% of NH₄OH; flow rate: 60 mL/min; gradient time=45 min]. The chiral purity was determined using SFC analytical method on Chiralcel OD-3 50 mm×4.6 mm×3 μm column; Mobile phase A: CO₂; Mobile phase B: EtOH with 0.05% of DEA; isocratic: 40% of Mobile Phase B; flow rate: 4.0 mL/min; backpressure: 1500 psi; column temperature: 35° C. to provide Example 63: peak 1 (retention time: 0.710 min, 100% ee) and peak 2 (retention time: 1.175 min, 100% ee).

Example 82 and 83\hat{ }: A crude material was purified using SFC [Column: DAICEL CHIRALPAK AD 250 mm×30 mm×10 μm; Mobile phase: 45% CO₂/55% isopropyl alcohol (0.1% NH₄OH); flow rate=80 mL/min]. The chiral purity was determined using SFC analytical method on Chiralpak AD-3 50 mm×4.6 mm×3 μm column; Mobile phase A: CO₂; Mobile phase B: isopropyl alcohol with 0.05% of DEA; isocratic: 40% of Mobile Phase B; flow rate: 4.0 mL/min; backpressure: 1500 psi; column temperature: 35° C. to provide Example 82: peak 1 (retention time: 1.233 min, 100% ee) and Example 83: peak 2 (retention time: 1.622 min, 97% ee).

Example 87 and 237\hat{ }: Example 62 was separated into its component isomers using SFC [Column: DAICEL CHIRALPAK AD 250 mm×30 mm×10 μm; Mobile phase: 50% CO₂/50% isopropyl alcohol (0.1% NH₄OH); flow rate=150 mL/min]. The chiral purity was determined using SFC analytical method on Chiralcel OD-3, 50 mm×4.6 mm×3 μm; Mobile phase A: CO₂; Mobile phase B: EtOH with 0.2% NH₃; isocratic gradient: 40% of Mobile Phase B; flow rate: 3.5 mL/min; column temperature: 35° C., backpressure: 1500 psi to provide Example 237: peak 1 ((SFC-MS) retention time: 2.293 min, 100% ee) and Example 87: peak 2 ((SFC-MS) retention time: 3.434 min, 100% ee)

Example 111\hat{ }: A crude material was purified using SFC [Column: DAICEL CHIRALCEL OD 250 mm×30 mm×10 μm; Mobile phase: 45% CO₂/55% EtOH (0.1% NH₄OH); flow rate=80 mL/min; gradient time=45 min]. The chiral purity was determined using SFC analytical method on Chiralpak AD-3, 50 mm×4.6 mm×3 μm column; Mobile phase A: CO₂; Mobile phase B: isopropyl alcohol with 0.05% DEA; isocratic gradient: 40% of Mobile Phase B; flow rate: 4.0 mL/min; column temperature: 35° C., backpressure: 1500 psi to provide Example 111: peak 1 ((SFC-MS) retention time: 4.224 min, 91% ee).

Example 112 and 128\hat{ }: Example 95 was separated into its component isomers using SFC [Column: DAICEL CHIRALCEL OD 250 mm×30 mm×10 μm; Mobile phase: 40% CO₂/60% EtOH (0.1% NH₄OH); flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH with 0.2% NH₃; isocratic gradient: 40% of Mobile Phase B; flow rate: 3.5 mL/min; column temperature: 35° C., backpressure: 1500 psi to provide Example 112: peak 1 ((SFC-MS) m/z (M+H)+=547.43 at retention time: 0.764 min, 100% ee) and Example 128: peak 2 ((SFC-MS) m/z (M+H)+=547.44 at retention time: 1.380 min, 100% ee).

Example 117 and 118\hat{ }: Example 116 was separated into its component isomers using SFC [Column: Chiral Technologies OJ-H 250 mm×21 mm×5 μm; Mobile phase A: 85% CO₂; Mobile Phase B: 15% MeOH (0.2% NH₄OH); isocratic: 15% of Mobile Phase B; flow rate=75 mL/min, gradient time=20 min, backpressure=120 bar]. The chiral purity was determined using SFC analytical method on Chiral Technologies OJ-H 100 mm×4.6 mm×5 μm column; Mobile phase A: 75% CO₂, Mobile phase B: 25% MeOH with 0.2% of NH₄OH; isocratic: 25% of Mobile Phase B; flow rate: 5.0 mL/min; backpressure: 120 bar, gradient time=5 min to provide Example 117: peak 1 ((SFC-MS) m/z (M+H)⁺=547.3 at retention time: 2.61 min) and Example 118: peak 2 ((SFC-MS) m/z (M+H)⁺=547.3 at retention time: 2.98 min).

Example 119 and 557\hat{ }: A crude material was purified using SFC [Column: DAICEL CHIRALCEL OD 250 mm×30 mm×10 μm; Mobile phase: 40% CO₂/60% EtOH with 0.1% of NH₄OH; flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH with 0.05% of DEA; isocratic: 40% of Mobile Phase B; flow rate: 4.0 mL/min; backpressure: 1500 psi; column temperature: 35° C. to provide Example 557: peak 1 (retention time: 0.784 min, 100% ee) and Example 119: peak 2 (retention time: 1.425 min, 100% ee).

Example 122\hat{ }: A crude material was purified using SFC [Column: DAICEL CHIRALCEL OJ 250 mm×30 mm×10 μm; Mobile phase: 65% CO₂/35% EtOH (0.1% NH₄OH); flow rate=80 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 50% of Mobile Phase B; flow rate: 3.5 mL/min; column temperature: 35° C., backpressure: 1500 psi to provide Example 122: peak 1 ((SFC-MS) m/z (M+H)⁺=528.44 at retention time: 1.045 min, 100% ee).

Example 123 and 124\hat{ }: A crude material was separated into its component isomers using SFC [Column: Chiral Technologies OJ-H 250 mm×21 mm×5 μm; Mobile phase A: 80% CO₂; Mobile Phase B: 20% MeOH with 0.2% of NH₄OH; isocratic: 20% of Mobile Phase B; flow rate=75 mL/min, gradient time=10 min, backpressure=120 bar]. The chiral purity was determined using SFC analytical method on Chiral Technologies OJ-H 100 mm×4.6 mm×5 μm column; Mobile phase A: 80% CO₂, Mobile phase B: 20% MeOH with 0.2% of NH₄OH; isocratic: 20% of Mobile Phase B; flow rate: 5.0 mL/min; backpressure: 120 bar, gradient time=5 min to provide Example 123: peak 1 ((SFC-MS) m/z (M+H)⁺=581.3 at retention time: 2.37 min) and Example 124: peak 2 ((SFC-MS) m/z (M+H)⁺=581.3 at retention time: 4.00 min).

Example 127 and 552\hat{ }: Example 532 was purified using SFC [Column: DAICEL CHIRALPAK AD 250 mm×30 mm×10 μm; Mobile phase: 50% CO₂ with ACN/50% MeOH with 0.1% NH₄OH; flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35° C., backpressure: 1500 psi to provide Example 127: peak 1 ((SFC-MS) m/z (M+H)⁺=548.51 at retention time: 1.085 min, 100% ee) and Example 552: peak 2 ((SFC-MS) m/z (M+H)⁺=548.51 at retention time: 1.489 min, 100% ee).

Example 131 and 336\hat{ }: Example 130 was purified using SFC [Column: DAICEL CHIRALCEL OD 250 mm×30 mm×10 μm; Mobile phase: 40% CO₂/60% EtOH with 0.1% of NH₄OH; flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH with 0.2% of NH₃; isocratic: 40% of Mobile Phase B; flow rate: 3.5 mL/min; backpressure: 1500 psi; column temperature: 35° C. to provide Example 131: peak 1 ((SFC-MS) m/z (M+H)⁺=533.44 at retention time: 0.973 min, 99% ee) and Example 336: peak 2 ((SFC-MS) m/z (M+H)⁺=533.45 at retention time: 1.841 min, 100% ee).

Example 190 and 191\hat{ }: Example 90 was separated into its component isomers using SFC [Column: DAICEL CHIRALPAK IG 250 mm×30 mm×10 μm; Mobile phase: 80% MeOH/20% ACN; flow rate=100 mL/min]. The chiral purity was determined using SFC analytical method on Chiralpak IG-3 50 mm×4.6 mm×3 μm column; Mobile phase A: MeOH (0.05% of DEA); Mobile phase B: ACN; isocratic gradient: 20% of Mobile Phase B; flow rate: 1.0 mL/min; column temperature: 35° C. to provide Example 190: peak 1 ((SFC-MS) retention time: 3.431 min, 100% ee) and Example 191: peak 2 ((SFC-MS) retention time: 4.646 min, 100% ee).

Example 203 and 204\hat{ }: Example 59 was separated into its component isomers using SFC [Column: DAICEL CHIRALCEL OD 250 mm×30 mm×10 μm; Mobile phase: 45% CO₂/55% EtOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: EtOH (0.05% of DEA); gradient: 5-40% of Mobile Phase B in 1.5 min and hold at 40% of Mobile Phase B for 1.0 min then 5% of Mobile phase B for 0.5 min; flow rate: 4.0 mL/min; column temperature: 35° C., backpressure: 1500 psi to provide Example 204: peak 1 ((SFC-MS) m/z (M+H)+=528.17 at retention time: 2.106 min, 100% ee) and Example 203: peak 2 ((SFC-MS) m/z (M+H)+=528.17 at retention time: 2.407 min, 99% ee).

Example 230 and 231\hat{ }: A mixture was separated into its component isomers using SFC [Column: Lux Cellulose 1 250 mm×21 mm×5 μm; Mobile phase: 35% MeOH (0.2% NH₄OH)/65% ACN; flow rate=75 mL/min, gradient time=20 min, backpressure=120 bar]. The chiral purity was determined using SFC analytical method on Lux Cellulose 1 100 mm×4.6 mm×5 μm column; Mobile phase A: MeOH (0.2% NH₄OH); Mobile phase B: ACN; isocratic gradient: 50% of Mobile Phase B; flow rate: 5.0 mL/min; backpressure: 120 bar, gradient time: 5 min to provide Example 230: peak 1 ((SFC-MS) m/z (M+H)⁺=545.2 at retention time: 2.92 min) and Example 231: peak 2 ((SFC-MS) m/z (M+H)⁺=545.3 at retention time: 3.87 min).

Example 232 and 233\hat{ }: Example 205 was separated into its component isomers using SFC [Column: DAICEL CHIRALCEL OD 250 mm×30 mm×10 μm; Mobile phase: 40% CO₂/60% EtOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: EtOH (0.05% of DEA); isocratic gradient: 40% of Mobile Phase B; flow rate: 4.0 mL/min; column temperature: 35° C., backpressure: 1500 psi to provide Example 233: peak 1 ((SFC-MS) retention time: 0.883 min, 100% ee) and Example 232: peak 2 ((SFC-MS) retention time: 1.583 min, 100% ee).

Example 234 and 235\hat{ }: Example 85 was separated into its component isomers using SFC [Column: Chiral Technologies AD-H 250 mm×30 mm×5 μm; Mobile phase: 55% CO₂/45% 2-propanol (0.2% isopropylamine); flow rate=80 mL/min, backpressure=120 bar, temperature=40° C.]. The chiral purity was determined using SFC analytical method on Chiral Technologies AD-H 250 mm×4.6 mm×5 μm column; Mobile phase A: CO₂; Mobile phase B: 2-propanol (0.2% isopropylamine); gradient: 5-60% of Mobile Phase B over 8.0 min then 5% of Mobile Phase B over 0.50 min; flow rate: 3.0 mL/min; column temperature: 40° C., backpressure: 120 bar to provide Example 234: peak 1 ((SFC-MS) retention time: 7.391 min, 100% ee) and Example 235: peak 2 ((SFC-MS) retention time: 7.794 min, 97% ee).

Example 250 and 251\hat{ }: A mixture was separated into its component isomers using SFC [Column: Lux Cellulose 3 250 mm×21 mm×5 μm; Mobile phase: 20% MeOH (0.2% NH₄OH)/80% ACN; flow rate=75 mL/min, gradient time=10 min, backpressure=120 bar]. The chiral purity was determined using SFC analytical method on Lux Cellulose 3 100 mm×4.6 mm×5 μm column; Mobile phase A: MeOH (0.2% NH₄OH); Mobile phase B: ACN; isocratic gradient: 75% of Mobile Phase B; flow rate: 5.0 mL/min; backpressure: 120 bar, gradient time: 5 min to provide Example 250: peak 1 ((SFC-MS) m/z (M+H)⁺=587.2 at retention time: 2.12 min) and Example 251: peak 2 ((SFC-MS) m/z (M+H)⁺=587.2 at retention time: 2.97 min).

Example 258 and 556\hat{ }: A crude material was purified using SFC [Column: DAICEL CHIRALCEL OD 250 mm×30 mm×10 μm; Mobile phase: 40% CO₂/60% EtOH (0.1% NH₄OH); flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH (0.05% DEA); isocratic gradient: 40% of Mobile Phase B; flow rate: 4.0 mL/min; column temperature: 35° C., backpressure: 1500 psi to provide Example 556: peak 1 ((SFC-MS) m/z (M+H)⁺=589.23 at retention time: 0.998 min, 100% ee) and Example 258: peak 2 ((SFC-MS) m/z (M+H)⁺=589.19 at retention time: 1.724 min, 100% ee).

Example 262 and 553\hat{ }: A crude material was purified using SFC [Column: DAICEL CHIRALCEL OD 250 mm×30 mm×10 μm; Mobile phase: 40% CO₂/60% EtOH (0.1% NH₄OH); flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH (0.05% DEA); isocratic gradient: 40% of Mobile Phase B; flow rate: 4.0 mL/min; column temperature: 35° C., backpressure: 1500 psi to provide Example 553: peak 1 ((SFC-MS) retention time: 0.487 min, 100% ee) and Example 262: peak 2 ((SFC-MS) retention time: 0.799 min, 100% ee).

Example 267 and 268\hat{ }: Example 249 was separated into its component isomers using SFC [Column: Pirkle Covalent (R,R) Whelk-01 250 mm×21.1 mm×5 μm; Mobile phase: 40% MeOH (0.2% NH₄OH)/60% ACN; flow rate=75 mL/min, gradient time=40 min, backpressure=120 bar]. The chiral purity was determined using SFC analytical method on Regis (R,R) Whelk-01 50 mm×5 mm×3 μm column; Mobile phase A: MeOH (0.2% NH₄OH); Mobile phase B: ACN; isocratic gradient: 55% of Mobile Phase B; flow rate: 5.0 mL/min; backpressure: 120 bar, gradient time: 10 min to provide Example 267: peak 1 ((SFC-MS) m/z (M+H)⁺=561.2 at retention time: 3.23 min) and Example 268: peak 2 ((SFC-MS) m/z (M+H)⁺=561.2 at retention time: 2.72 min).

Example 269 and 270\hat{ }: Example 260 was purified using SFC [Column: DAICEL CHIRALPAK IF 250 mm×30 mm×10 μm; Mobile phase: 30% heptane/70% (4:1, isopropyl alcohol:ACN) with 0.1% NH₄OH; flow rate=80 mL/min]. The chiral purity was determined using SFC analytical method on Chiralpak IF 100 mm×4.6 mm×3 μm column; Mobile phase A: hexane (0.1% DEA); Mobile phase B: (2:1, isopropyl alcohol:ACN); isocratic gradient: 60% of Mobile Phase B; flow rate: 1.0 mL/min; column temperature: 35° C. to provide Example 269: peak 1 ((SFC-MS) retention time: 3.315 min, 100% ee) and Example 270: peak 2 ((SFC-MS) retention time: 6.412 min, 100% ee) Example 271 and 272\hat{ }: Example 265 was purified using SFC [Column: DAICEL CHIRALCEL OD 250 mm×30 mm×10 μm; Mobile phase: 40% CO₂/60% EtOH (0.1% NH₄OH); flow rate=80 mL/min]. The chiral purity was determined using SFC analytical method on Chiralpak AD-3 50 mm×4.6 mm×3 μm column; Mobile phase A: CO₂; Mobile phase B: EtOH (0.2% NH₃); isocratic gradient: 40% of Mobile Phase B; flow rate: 4.0 mL/min; column temperature: 35° C., backpressure: 1500 psi to provide Example 271: peak 1 ((SFC-MS) m/z (M+H)⁺=547.21 at retention time: 1.545 min, 100% ee) and Example 272: peak 2 ((SFC-MS) m/z (M+H)⁺=547.21 at retention time: 3.340 min, 100% ee).

Example 273\hat{ }: A crude material was purified using SFC [Column: DAICEL CHIRALPAK AD 250 mm×30 mm×10 μm; Mobile phase: 50% CO₂/50% isopropyl alcohol (0.1% NH₄OH); flow rate=150 mL/min; gradient time=4 min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35° C., backpressure: 1500 psi to provide Example 273: peak 1 ((SFC-MS) m/z (M+H)⁺=520.15 at retention time: 1.272 min, 100% ee).

Example 274 and 275\hat{ }: Example 68 was purified using SFC [Column: CHIRALPAK IH 250 mm×30 mm×10 μm; Mobile phase: 45% CO₂/55% EtOH (0.1% NH₄OH); flow rate=140 mL/min, gradient time=4 min]. The chiral purity was determined using SFC analytical method on Chiralpak AS-3 50 mm×4.6 mm×3 μm column; Mobile phase A: CO₂; Mobile phase B: EtOH (0.2% NH₃); gradient: 5-40% of Mobile Phase B in 2.0 min then hold for 40% of Mobile Phase B in 1.2 min then 5% of Mobile Phase B for 0.8 min; flow rate: 3.0 mL/min; column temperature: 35° C., backpressure: 1500 psi to provide Example 275: peak 1 ((SFC-MS) m/z (M+H)⁺=545.16 at retention time: 2.593 min, 99% ee) and Example 274: peak 2 ((SFC-MS) m/z (M+H)⁺=545.17 at retention time: 3.311 min, 100% ee).

Example 276 and 277\hat{ }: Example 255 was separated into its component isomers using SFC [Column: Lux Cellulose 3 250 mm×21 mm×5 μm; Mobile phase: 15% MeOH (0.2% NH₄OH)/85% ACN; flow rate=75 mL/min, gradient time=20 min, backpressure=120 bar]. The chiral purity was determined using SFC analytical method on Lux Cellulose 3 100 mm×4.6 mm×5 μm column; Mobile phase A: MeOH (0.2% NH₄OH); Mobile phase B: ACN; isocratic gradient: 80% of Mobile Phase B; flow rate: 5.0 mL/min; backpressure: 120 bar, gradient time: 5 min to provide Example 276: peak 1 ((SFC-MS) m/z (M+H)⁺=601.2 at retention time: 2.90 min) and Example 277: peak 2 ((SFC-MS) m/z (M+H)⁺=601.2 at retention time: 3.90 min).

Example 280 and 281\hat{ }: A mixture was separated into its component isomers using SFC [Column: Lux Cellulose 1 250 mm×21 mm×5 μm; Mobile phase: 40% MeOH (0.2% NH₄OH)/60% ACN; flow rate=75 mL/min, gradient time=40 min, backpressure=120 bar]. The chiral purity was determined using SFC analytical method on Lux Cellulose 1 100 mm×4.6 mm×5 μm column; Mobile phase A: MeOH (0.2% NH₄OH); Mobile phase B: ACN; isocratic gradient: 40% of Mobile Phase B; flow rate: 5.0 mL/min; backpressure: 120 bar, gradient time: 10 min to provide Example 280: peak 1 ((SFC-MS) m/z (M+H)⁺=531.2 at retention time: 4.84 min) and Example 281: peak 2 ((SFC-MS) m/z (M+H)⁺=531.2 at retention time: 6.81 min).

Example 285 and 551\hat{ }: A mixture was purified using SFC [Column: YMC-IB 250 mm×30 mm×10 μm; Mobile phase: 70% heptane/30% (4:1, isopropyl alcohol:ACN) with 0.1% NH₄OH; flow rate=100 mL/min]. The chiral purity was determined using SFC analytical method on Chiralpak IB-N 100 mm×4.6 mm×3 μm column; Mobile phase A: hexane (0.1% DEA); Mobile phase B: (2:1, isopropyl alcohol:ACN); isocratic gradient: 40% of Mobile Phase B; flow rate: 1.0 mL/min; column temperature: 35° C. to provide Example 551: peak 1 ((SFC-MS) retention time: 4.066 min, 100% ee) and Example 285: peak 2 ((SFC-MS) retention time: 4.601 min, 99% ee).

Example 287\hat{ }: A crude material was purified using SFC [Column: DAICEL CHIRALCEL OD 250 mm×30 mm×10 μm; Mobile phase: 40% CO₂/60% EtOH (0.1% NH₄OH); flow rate=80 mL/min; gradient time=45 min] to provide Example 287. The chiral purity was determined using SFC analytical method on Chiralcel OD-3 50 mm×4.6 mm×3 μm column; Mobile phase A: CO₂; Mobile phase B: EtOH with 0.2% of NH₃; gradient: 5-40% of Mobile Phase B over 2.0 min and held at 40% of Mobile Phase B for 1.2 min then % of Mobile Phase B for 0.8 min; flow rate: 4.0 mL/min; backpressure: 1500 psi; column temperature: 35° C. to provide Example 287: peak 1 ((SFC-MS) m/z (M+H)⁺=559.20 at retention time: 2.903 min, 100% ee).

Example 303 and 304\hat{ }: Example 282 was separated into its component isomers using SFC [Column: Chiral Technologies OJ-H 250 mm×21 mm×5 μm; Mobile phase: 25% MeOH (0.2% NH₄OH)/75% ACN; flow rate=75 mL/min, gradient time=10 min, backpressure=120 bar]. The chiral purity was determined using SFC analytical method on Chiral Technologies OJ-H 100 mm×4.6 mm×5 μm column; Mobile phase A: MeOH (0.2% NH₄OH); Mobile phase B: ACN; isocratic gradient: 70% of Mobile Phase B; flow rate: 5.0 mL/min; backpressure: 120 bar, gradient time: 5 min to provide Example 303: peak 1 ((SFC-MS) m/z (M+H)⁺=587.2 at retention time: 1.92 min) and Example 304: peak 2 ((SFC-MS) m/z (M+H)⁺=587.2 at retention time: 2.74 min).

Example 307 and 550\hat{ }: A mixture was purified using SFC [Column: DAICEL CHIRALCEL OD 250 mm×30 mm×10 μm; Mobile phase: 50% CO₂/50% MeOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: MeOH (0.2% NH₃); gradient: 5-40% of Mobile Phase B in 1.5 min and hold at 40% of Mobile Phase B for 1.0 min then 5% of Mobile phase B for 0.5 min; flow rate: 4.0 mL/min; column temperature: 35° C., backpressure: 1500 psi to provide Example 550: peak 1 ((SFC-MS) m/z (M+H)⁺=587.19 at retention time: 1.669 min, 100% ee) and Example 307: peak 2 ((SFC-MS) m/z (M+H)⁺=587.16 at retention time: 1.945 min, 100% ee).

Example 310 and 311\hat{ }: Example 246 was separated into its component isomers using SFC [Column: Chiral Technologies IB 250 mm×21 mm×5 μm; Mobile phase: 40% MeOH (0.2% NH₄OH)/60% ACN; flow rate=75 mL/min, gradient time=10 min, backpressure=120 bar]. The chiral purity was determined using SFC analytical method on Chiral Technologies IB 100 mm×4.6 mm×5 μm column; Mobile phase A: MeOH (0.2% NH₄OH); Mobile phase B: ACN; isocratic gradient: 50% of Mobile Phase B; flow rate: 5.0 mL/min; backpressure: 120 bar, gradient time: 5 min to provide Example 310: peak 1 ((SFC-MS) m/z (M+H)⁺=561.3 at retention time: 2.14 min) and Example 311: peak 2 ((SFC-MS) m/z (M+H)⁺=561.3 at retention time: 3.12 min).

Example 312 and 313\hat{ }: Example 115 was separated into its component isomers using SFC [Column: Chiral Technologies IB 250 mm×21 mm×5 μm; Mobile phase: 40% MeOH (0.2% NH₄OH)/60% ACN; flow rate=75 mL/min, gradient time=10 min, backpressure=120 bar]. The chiral purity was determined using SFC analytical method on Chiral Technologies IB 100 mm×4.6 mm×5 μm column; Mobile phase A: MeOH (0.2% NH₄OH); Mobile phase B: ACN; isocratic gradient: 50% of Mobile Phase B; flow rate: 5.0 mL/min; backpressure: 120 bar, gradient time: 5 min to provide Example 312: peak 1 ((SFC-MS) m/z (M+H)⁺=547.3 at retention time: 2.40 min) and Example 313: peak 2 ((SFC-MS) m/z (M+H)⁺=547.3 at retention time: 3.61 min).

Example 316 and 317\hat{ }: Example 314 was separated into its component isomers using SFC [Column: Lux Cellulose 1 250 mm×21 mm×5 μm; Mobile phase: 35% MeOH (0.2% NH₄OH)/65% ACN; flow rate=75 mL/min, gradient time=10 min, backpressure=120 bar]. The chiral purity was determined using SFC analytical method on Lux Cellulose 1 100 mm×4.6 mm×5 μm column; Mobile phase A: MeOH (0.2% NH₄OH); Mobile phase B: ACN; isocratic gradient: 60% of Mobile Phase B; flow rate: 5.0 mL/min; backpressure: 120 bar, gradient time: 5 min to provide Example 316: peak 2 ((SFC-MS) m/z (M+H)⁺=547.2 at retention time: 2.40 min) and Example 317: peak 3 ((SFC-MS) m/z (M+H)⁺=547.2 at retention time: 2.94 min).

Example 318 and 319\hat{ }: A crude material was purified using SFC [Column: DAICEL CHIRALPAK AD 250 mm×30 mm×10 μm; Mobile phase: 50% CO₂/50% isopropyl alcohol (0.1% NH₄OH); flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35° C., backpressure: 1500 psi to provide Example 318: peak 1 ((SFC-MS) m/z (M+H)⁺=561.20 at retention time: 1.188 min, 100% ee) and Example 319: peak 2 ((SFC-MS) m/z (M+H)⁺=561.22 at retention time: 1.900 min, 99% ee).

Example 335 and 549\hat{ }: A crude material was purified using SFC [Column: DAICEL CHIRALPAK AD 250 mm×30 mm×10 μm; Mobile phase: 50% CO₂/50% EtOH (0.1% NH₄OH); flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35° C., backpressure: 1500 psi to provide Example 549: peak 1 ((SFC-MS) m/z (M+H)⁺=528.18 at retention time: 0.811 min, 99% ee) and Example 335: peak 2 ((SFC-MS) m/z (M+H)⁺=528.25 at retention time: 1.497 min, 100% ee).

Example 347 and 348\hat{ }: Example 358 was purified using SFC [Column: DAICEL CHIRALPAK AD 250 mm×30 mm×10 μm; Mobile phase: 50% CO₂/50% isopropyl alcohol (0.1% NH₄OH); flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35° C., backpressure: 1500 psi to provide Example 347: peak 1 ((SFC-MS) m/z (M+H)⁺=558.17 at retention time: 1.067 min, 100% ee) and Example 348: peak 2 ((SFC-MS) m/z (M+H)⁺=558.21 at retention time: 1.503 min, 98% ee).

Example 352 and 353\hat{ }: The crude mixture was separated into its component isomers using SFC [Column: Chiral Technologies IH 250 mm×21 mm×5 μm; Mobile phase: 35% MeOH (0.2% NH₄OH)/65% ACN; flow rate=75 mL/min, gradient time=10 min, backpressure=120 bar]. The chiral purity was determined using SFC analytical method on Chiral Technologies IH 100 mm×4.6 mm×5 μm column; Mobile phase A: MeOH (0.2% NH₄OH); Mobile phase B: ACN; isocratic gradient: 60% of Mobile Phase B; flow rate: 5.0 mL/min; backpressure: 120 bar, gradient time: 5 min to provide Example 352: peak 1 ((SFC-MS) m/z (M+H)⁺=547.2 at retention time: 1.97 min) and Example 353: peak 2 ((SFC-MS) m/z (M+H)⁺=547.3 at retention time: 3.05 min).

Example 354 and 355\hat{ }: Example 315 was separated into its component isomers using SFC [Column: Chiral Technologies OD-H 250 mm×21 mm×5 μm; Mobile phase: 25% MeOH (0.2% NH₄OH)/75% ACN; flow rate=75 mL/min, gradient time=20 min, backpressure=120 bar]. The chiral purity was determined using SFC analytical method on Chiral Technologies OD-H 100 mm×4.6 mm×5 μm column; Mobile phase A: MeOH (0.2% NH₄OH); Mobile phase B: ACN; isocratic gradient: 60% of Mobile Phase B; flow rate: 5.0 mL/min; backpressure: 120 bar, gradient time: 5 min to provide Example 354: peak 1 ((SFC-MS) m/z (M+H)⁺=561.4 at retention time: 2.67 min) and Example 355: peak 2 ((SFC-MS) m/z (M+H)⁺=561.3 at retention time: 3.19 min).

Example 356 and 357\hat{ }: The crude mixture was separated into its component isomers using SFC [Column: Chiral Technologies OJ-H 250 mm×21 mm×5 μm; Mobile phase: 20% MeOH (0.2% NH₄OH)/80% ACN; flow rate=75 mL/min, gradient time=10 min, backpressure=120 bar]. The chiral purity was determined using SFC analytical method on Chiral Technologies OJ-H 100 mm×4.6 mm×5 μm column; Mobile phase A: MeOH (0.2% NH₄OH); Mobile phase B: ACN; isocratic gradient: 75% of Mobile Phase B; flow rate: 5.0 mL/min; backpressure: 120 bar, gradient time: 5 min to provide Example 356: peak 1 ((SFC-MS) m/z (M+H)⁺=572.2 at retention time: 1.78 min) and Example 357: peak 2 ((SFC-MS) m/z (M+H)⁺=572.2 at retention time: 3.51 min).

Example 359 and 360\hat{ }: Example 351 was purified using SFC [Column: DAICEL CHIRALPAK IF 250 mm×30 mm×10 μm; Mobile phase: 50% heptane/50% (4:1, isopropyl alcohol:ACN); flow rate=100 mL/min]. The chiral purity was determined using SFC analytical method on Chiralpak IF 100 mm×4.6 mm×3 μm column; Mobile phase A: hexane (0.1% DEA); Mobile phase B: (2:1, isopropyl alcohol:ACN); isocratic gradient: 60% of Mobile Phase B; flow rate: 1.0 mL/min; column temperature: 35° C. to provide Example 359: peak 1 ((SFC-MS) retention time: 3.568 min, 100% ee) and Example 360: peak 2 ((SFC-MS) retention time: 4.561 min, 98% ee).

Example 361 and 362\hat{ }: Example 322 was purified using SFC [Column: DAICEL CHIRALCEL OD 250 mm×30 mm×10 μm; Mobile phase: 45% CO₂/55% EtOH (0.1% NH₄OH); flow rate=80 mL/min]. The chiral purity was determined using SFC analytical method on Chiralcel OD-3 50 mm×4.6 mm×3 μm column; Mobile phase A: CO₂; Mobile phase B: EtOH with 0.2% of NH₃; isocratic: 40% of Mobile Phase B; flow rate: 4.0 mL/min; backpressure: 1500 psi; column temperature: 35° C. to provide Example 361: peak 1 ((SFC-MS) m/z (M+H)⁺=532.18 at retention time: 0.818 min, 100% ee) and Example 362: peak 2 ((SFC-MS) m/z (M+H)⁺=532.21 at retention time: 1.180 min, 99% ee).

Example 391 and 392\hat{ }: The crude mixture was separated into its component isomers using SFC [Column: Chiral Technologies OJ-H 250 mm×21 mm×5 μm; Mobile phase: 30% MeOH (0.2% NH₄OH)/70% ACN; flow rate=75 mL/min, gradient time=10 min, backpressure=120 bar]. The chiral purity was determined using SFC analytical method on Chiral Technologies OJ-H 100 mm×4.6 mm×5 μm column; Mobile phase A: MeOH (0.2% NH₄OH); Mobile phase B: ACN; isocratic gradient: 65% of Mobile Phase B; flow rate: 5.0 mL/min; backpressure: 120 bar, gradient time: 5 min to provide Example 391: peak 1 ((SFC-MS) m/z (M+H)⁺=573.2 at retention time: 1.93 min) and Example 392: peak 2 ((SFC-MS) m/z (M+H)⁺=573.2 at retention time: 3.20 min).

Example 393 and 394\hat{ }: Example 339 was purified using SFC [Column: DAICEL CHIRALCEL OJ 250 mm×30 mm×10 μm; Mobile phase: 45% CO₂/55% EtOH (0.1% NH₄OH); flow rate=80 mL/min]. The chiral purity was determined using SFC analytical method on Chiralcel OJ-3 50 mm×4.6 mm×3 μm column; Mobile phase A: CO₂; Mobile phase B: EtOH (0.2% NH₃); isocratic gradient: 40% of Mobile Phase B; flow rate: 3.0 mL/min; column temperature: 35° C., backpressure: 1500 psi to provide Example 393: peak 1 ((SFC-MS) m/z (M+H)⁺=546.64 at retention time: 0.895 min, 100% ee) and Example 394: peak 2 ((SFC-MS) m/z (M+H)⁺=546.63 at retention time: 1.939 min, 99% ee).

Example 428 and 429\hat{ }: Example 399 was purified using SFC [Column: DAICEL CHIRALCEL OD 250 mm×30 mm×10 μm; Mobile phase: 45% CO₂/55% EtOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: EtOH (0.2% NH₃); isocratic gradient: 40% of Mobile Phase B; flow rate: 4.0 mL/min; column temperature: 35° C., backpressure: 1500 psi to provide Example 428: peak 1 ((SFC-MS) m/z (M+H)⁺=532.22 at retention time: 0.636 min, 100% ee) and Example 429: peak 2 ((SFC-MS) m/z (M+H)⁺=532.21 at retention time: 1.130 min, 100% ee).

Example 444 and 445\hat{ }: Example 55 was purified using SFC [Column: ChiralPak IH 250 mm×30 mm×10 μm; Mobile phase: 50% CO₂/50% EtOH (0.1% NH₄OH); flow rate=150 mL/min, gradient time=4 min]. The chiral purity was determined using SFC analytical method on Chiralpak AS-3 50 mm×4.6 mm×3 μm column; Mobile phase A: CO₂; Mobile phase B: EtOH (0.2% NH₃); isocratic gradient: 40% of Mobile Phase B; flow rate: 3.0 mL/min; column temperature: 35° C., backpressure: 1500 psi to provide Example 444: peak 1 ((SFC-MS) m/z (M+H)⁺=519.62 at retention time: 1.191 min, 100% ee) and Example 445: peak 2 ((SFC-MS) m/z (M+H)⁺=519.62 at retention time: 1.728 min, 99% ee).

Example 446 and 447\hat{ }: The crude mixture was separated into its component isomers using SFC [Column: Chiral Technologies IA 250 mm×21 mm×5 μm; Mobile phase: 40% MeOH (0.2% NH₄OH)/60% ACN; flow rate=75 mL/min, gradient time=20 min, backpressure=120 bar]. The chiral purity was determined using SFC analytical method on Chiral Technologies IA 100 mm×4.6 mm×5 μm column; Mobile phase A: MeOH (0.2% NH₄OH); Mobile phase B: ACN; isocratic gradient: 45% of Mobile Phase B; flow rate: 5.0 mL/min; backpressure: 120 bar, gradient time: 10 min to provide Example 446: peak 1 ((SFC-MS) m/z (M+H)⁺=548.3 at retention time: 2.33 min) and Example 447: peak 2 ((SFC-MS) m/z (M+H)⁺=548.3 at retention time: 6.75 min).

Example 448 and 449\hat{ }: The crude mixture was separated into its component isomers using SFC [Column: Chiral Technologies IA 250 mm×21 mm×5 μm; Mobile phase: 35% MeOH (0.2% NH₄OH)/65% ACN; flow rate=75 mL/min, gradient time=20 min, backpressure=120 bar]. The chiral purity was determined using SFC analytical method on Chiral Technologies IA 100 mm×4.6 mm×5 μm column; Mobile phase A: MeOH (0.2% NH₄OH); Mobile phase B: ACN; isocratic gradient: 45% of Mobile Phase B; flow rate: 5.0 mL/min; backpressure: 120 bar, gradient time: 10 min to provide Example 448: peak 1 ((SFC-MS) m/z (M+H)⁺=562.3 at retention time: 1.96 min) and Example 449: peak 2 ((SFC-MS) m/z (M+H)⁺=562.3 at retention time: 4.38 min).

Example 461 and 462\hat{ }: Example 459 was separated into its component isomers using chiral HPLC purification [Column: C₁₈ 150 mm×40 mm; Mobile phase: H₂O (NH₄OH—NH₄HCO₃)/ACN; gradient=13-53% of Mobile Phase B; flow rate=60 mL/min, gradient time=9 min]. The chiral purity was determined using SFC analytical method on Chiralpak AD-3 50 mm×4.6 mm×3 μm column; Mobile phase A: CO₂; Mobile phase B: EtOH (0.2% NH₃); isocratic gradient: 40% of Mobile Phase B; flow rate: 3.5 mL/min; column temperature: 35° C., backpressure: 1500 psi to provide Example 461: peak 1 ((SFC-MS) m/z (M+H)⁺=602.71 at retention time: 0.837 min, 100% ee) and Example 462: peak 2 ((SFC-MS) m/z (M+H)⁺=602.70 at retention time: 1.799 min, 100% ee).

Example 464 and 465\hat{ }: Example 460 was purified using SFC [Column: DAICEL CHIRALPAK AD 250 mm×30 mm×10 μm; Mobile phase: 50% CO₂/50% EtOH (0.1% NH₄OH); flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 3.5 mL/min; column temperature: 35° C., backpressure: 1500 psi to provide Example 464: peak 1 ((SFC-MS) m/z (M+H)⁺=588.69 at retention time: 0.715 min, 100% ee) and Example 465: peak 2 ((SFC-MS) m/z (M+H)⁺=588.67 at retention time: 2.128 min, 98% ee).

Example 467 and 468\hat{ }: Example 463 was purified using SFC [Column: DAICEL CHIRALCEL OD 250 mm×30 mm×10 μm; Mobile phase: 45% CO₂/55% MeOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: MeOH (0.2% NH₃); isocratic gradient: 40% of Mobile Phase B; flow rate: 4.0 mL/min; column temperature: 35° C., backpressure: 1500 psi to provide Example 467: peak 1 ((SFC-MS) m/z (M+H)⁺=558.47 at retention time: 0.408 min, 100% ee) and Example 468: peak 2 ((SFC-MS) m/z (M+H)⁺=558.48 at retention time: 0.707 min, 99% ee).

Example 474 and 475\hat{ }: Example 472 was purified using SFC [Column: DAICEL CHIRALPAK AD 250 mm×30 mm×10 μm; Mobile phase: 40% CO₂/60% MeOH (0.1% NH₄OH); flow rate=80 mL/min, gradient time=45 min]. The chiral purity was determined using SFC analytical method on Chiralpak AD-3 50 mm×4.6 mm×3 μm column; Mobile phase A: CO₂; Mobile phase B: MeOH (0.2% NH₃); isocratic gradient: 40% of Mobile Phase B; flow rate: 3.5 mL/min; column temperature: 35° C., backpressure: 1500 psi to provide Example 474: peak 1 ((SFC-MS) m/z (M+H)⁺=549.68 at retention time: 0.836 min, 100% ee) and Example 475: peak 2 ((SFC-MS) m/z (M+H)⁺=549.68 at retention time: 2.047 min, 100% ee).

Example 493 and 494\hat{ }: The crude mixture was purified using SFC [Column: DAICEL CHIRALCEL OD 250 mm×30 mm×10 μm; Mobile phase: 40% CO₂/60% MeOH (0.1% NH₄OH); flow rate=70 mL/min, gradient time=45 min, column temperature=40° C., backpressure=100 bar]. The chiral purity was determined using SFC analytical method on Chiralcel OD-3 50 mm×4.6 mm×3 μm column; Mobile phase A: CO₂; Mobile phase B: MeOH (0.2% NH₃); isocratic gradient: 40% of Mobile Phase B; flow rate: 3.5 mL/min; column temperature: 35° C., backpressure: 1500 psi to provide Example 493: peak 1 ((SFC-MS) m/z (M+H)⁺=583.71 at retention time: 0.784 min, 100% ee) and Example 494: peak 2 ((SFC-MS) m/z (M+H)⁺=583.69 at retention time: 1.855 min, 100% ee).

Example 506 and 507\hat{ }: The crude mixture was purified using SFC [Column: DAICEL CHIRALPAK AS 250 mm×30 mm×10 μm; Mobile phase: 50% CO₂/50% (4:1, EtOH:ACN) (0.1% NH₄OH); flow rate=150 mL/min, gradient time=40 min]. The chiral purity was determined using SFC analytical method on Chiralpak AS-3 100 mm×4.6 mm×3 μm column; Mobile phase A: CO₂; Mobile phase B: EtOH (0.2% NH₃); isocratic gradient: 40% of Mobile Phase B; flow rate: 2.8 mL/min; column temperature: 35° C., backpressure: 1500 psi to provide Example 506: peak 1 ((SFC-MS) m/z (M+H)⁺=547.25 at retention time: 1.151 min, 100% ee) and Example 507: peak 2 ((SFC-MS) m/z (M+H)⁺=547.22 at retention time: 2.043 min, 100% ee).

Example 508 and 509\hat{ }: The crude mixture was purified using SFC [Column: DAICEL CHIRALPAK AS 250 mm×30 mm×10 μm; Mobile phase: 50% CO₂/50% (4:1, EtOH:ACN) (0.1% NH₄OH); flow rate=150 mL/min, gradient time=60 min]. The chiral purity was determined using SFC analytical method on Chiralpak AS-3 100 mm×4.6 mm×3 μm column; Mobile phase A: CO₂; Mobile phase B: EtOH (0.2% NH₃); isocratic gradient: 40% of Mobile Phase B; flow rate: 2.8 mL/min; column temperature: 35° C., backpressure: 1500 psi to provide Example 508: peak 1 ((SFC-MS) m/z (M+H)⁺=548.26 at retention time: 1.285 min, 100% ee) and Example 509: peak 2 ((SFC-MS) m/z (M+H)⁺=548.31 at retention time: 3.523 min, 100% ee).

Example 510 and 511\hat{ }: The crude mixture was purified using SFC [Column: DAICEL CHIRALPAK AS 250 mm×30 mm×10 μm; Mobile phase: 45% CO₂/55% (1:1, EtOH:ACN) (0.1% NH₄OH); flow rate=140 mL/min, column temperature=40° C., backpressure=100 bar]. The chiral purity was determined using SFC analytical method on Chiralpak AS-3 50 mm×4.6 mm×3 μm column; Mobile phase A: CO₂; Mobile phase B: EtOH (0.2% NH₃); isocratic gradient: 50% of Mobile Phase B; flow rate: 2.5 mL/min; column temperature: 35° C., backpressure: 1500 psi to provide Example 510: peak 1 ((SFC-MS) m/z (M+H)⁺=534.55 at retention time: 0.702 min, 100% ee) and Example 511: peak 2 ((SFC-MS) m/z (M+H)⁺=534.58 at retention time: 1.561 min, 100% ee).

Example 514 and 515\hat{ }: The crude mixture was purified using SFC [Column: DAICEL CHIRALPAK AS 250 mm×30 mm×10 μm; Mobile phase: 45% CO₂/55% EtOH (0.1% NH₄OH); flow rate=140 mL/min, column temperature=40° C., backpressure=100 bar]. The chiral purity was determined using SFC analytical method on Chiralpak AS-3 50 mm×4.6 mm×3 μm column; Mobile phase A: CO₂; Mobile phase B: EtOH (0.2% NH₃); isocratic gradient: 40% of Mobile Phase B; flow rate: 3.0 mL/min; column temperature: 35° C., backpressure: 1500 psi to provide Example 514: peak 1 ((SFC-MS) m/z (M+H)⁺=533.56 at retention time: 0.979 min, 100% ee) and Example 515: peak 2 ((SFC-MS) m/z (M+H)⁺=533.56 at retention time: 1.597 min, 99% ee).

Example 527 and 528\hat{ }: A crude material was purified using SFC [Column: DAICEL CHIRALPAK AS 250 mm×30 mm×10 μm; Mobile phase: 45% CO₂/55% EtOH (0.1% NH₄OH); flow rate=140 mL/min, backpressure=100 bar, column temperature=40° C.]. The chiral purity was determined using SFC analytical method on Chiralpak AS-3 50 mm×4.6 mm×3 μm column; Mobile phase A: CO₂; Mobile phase B: EtOH (0.2% NH₃); isocratic gradient: 50% of Mobile Phase B; flow rate: 2.5 mL/min; column temperature: 35° C., backpressure: 1500 psi to provide Example 527: peak 1 ((SFC-MS) m/z (M+H)⁺=561.59 at retention time: 0.509 min, 100% ee) and Example 528: peak 2 ((SFC-MS) m/z (M+H)⁺=561.59 at retention time: 1.377 min, 100% ee).

Example 529\hat{ }: Example 524 was separated into its component isomers using SFC [Column: Phenomenex Lux Cellulose 1 250 mm×21 mm×5 μm; Mobile phase: 30% MeOH/70% ACN; flow rate=75 mL/min, gradient time=20 min, backpressure=120 bar, column temperature=50° C.]. The chiral purity was determined using SFC analytical method on Lux Cellulose 1 100 mm×4.6 mm×5 μm column; Mobile phase A: MeOH; Mobile phase B: ACN; isocratic gradient: 60% of Mobile Phase B; flow rate: 5.0 mL/min; backpressure: 120 bar, gradient time: 5 min to provide peak 1 ((SFC-MS) m/z (M+H)⁺=561.2 at retention time: 3.28 min) and Example 529: peak 2 ((SFC-MS) m/z (M+H)⁺=561.2 at retention time: 3.40 min).

Example 538 and 539\hat{ }: Example 537 was purified using SFC [Column: DAICEL CHIRALCEL OD 250 mm×30 mm×10 μm; Mobile phase: 40% CO₂/60% EtOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: EtOH (0.2% NH₃); isocratic gradient: 40% of Mobile Phase B; flow rate: 4.0 mL/min; column temperature: 35° C., backpressure: 1500 psi to provide Example 539: peak 1 ((SFC-MS) m/z (M+H)⁺=546.24 at retention time: 0.607 min, 100% ee) and Example 538: peak 2 ((SFC-MS) m/z (M+H)⁺=546.22 at retention time: 1.034 min, 100% ee).

Example 547 and 548\hat{ }: Example 546 was separated into its component isomers using SFC [Column: Chiral Technologies OD-H 250 mm×21 mm×5 μm; Mobile phase: 40% CO₂/60% MeOH (0.2% NH₄OH); flow rate=75 mL/min, backpressure=120 bar, gradient time=10 min]. The chiral purity was determined using SFC analytical method on Chiral Technologies OD-H 100 mm×4.6 mm×5 μm column; Mobile phase A: CO₂; Mobile phase B: MeOH (0.2% NH₄OH); isocratic gradient: 50% of Mobile Phase B; flow rate: 5.0 mL/min; backpressure: 120 bar to provide Example 548: peak 1 ((SFC-MS) m/z (M+H)⁺=601.3 at retention time: 1.99 min). and Example 547: peak 2 ((SFC-MS) m/z (M+H)⁺=601.3 at retention time: 3.94 min).

Example 667 and 668\hat{ }: A mixture was separated into its component isomers using SFC [Column: Chiral Technologies AD-H 250 mm×30 mm×5 μm; Mobile phase: 60% CO₂/40% 2-propanol (0.2% isopropylamine); flow rate=100 mL/min, backpressure=100 bar, temperature=40° C.]. The chiral purity was determined using SFC analytical method on Chiral Technologies AD-H 250 mm×4.6 mm×5 μm column; Mobile phase A: CO₂; Mobile phase B: 2-propanol (0.2% isopropylamine); gradient: 5-100% of Mobile Phase B over 7.50 min then 5% of Mobile Phase B over 0.50 min; flow rate: 3.0 mL/min; column temperature: 40° C., backpressure: 100 bar to provide Example 668: peak 1 ((SFC-MS) retention time: 6.547 min, 99% ee) and Example 667: peak 2 ((SFC-MS) retention time: 6.951 min, 96% ee).

Example 670 and 671\hat{ }: A mixture was separated into its component isomers using SFC [Column: Chiral Technologies AS-H 250 mm×21 mm×5 μm; Mobile phase A: 25% CO₂; Mobile Phase B: 75% MeOH (0.2% NH₄OH); isocratic: 75% of Mobile Phase B; flow rate=75 mL/min, gradient time=20 min, backpressure=120 bar]. The chiral purity was determined using SFC analytical method on Chiral Technologies AS-H 100 mm×4.6 mm×5 μm column; Mobile phase A: 25% CO₂, Mobile phase B: 75% MeOH with 0.2% of NH₄OH; isocratic: 75% of Mobile Phase B; flow rate: 5.0 mL/min; backpressure: 120 bar, gradient time=20 min to provide Example 671: peak 1 ((SFC-MS) m/z (M+H)⁺=645.4 at retention time: 8.71 min) and Example 670: peak 2 ((SFC-MS) m/z (M+H)⁺=645.3 at retention time: 13.48 min).

Example 672 and 673\hat{ }: Example 710 was separated into its component isomers using SFC [Column: Chiral Technologies IB 250 mm×21 mm×5 μm; Mobile phase A: 35% CO₂; Mobile Phase B: 65% MeOH (0.2% NH₄OH); isocratic: 65% of Mobile Phase B; flow rate=75 mL/min, gradient time=20 min, backpressure=120 bar]. The chiral purity was determined using SFC analytical method on Chiral Technologies IB 100 mm×4.6 mm×5 μm column; Mobile phase A: 40% CO₂, Mobile phase B: 60% MeOH with 0.2% of NH₄OH; isocratic: 60% of Mobile Phase B; flow rate: 5.0 mL/min; backpressure: 120 bar, gradient time=10 min to provide Example 673: peak 1 ((SFC-MS) m/z (M+H)⁺=631.3 at retention time: 5.53 min) and Example 672: peak 2 ((SFC-MS) m/z (M+H)⁺=631.3 at retention time: 6.86 min).

Example 692 and 693\hat{ }: Example 701 was separated into its component isomers using SFC [Column: DAICEL CHIRALCEL OD 250 mm×30 mm×10 μm; Mobile phase: 50% CO₂/50% EtOH (0.1% NH₄OH); isocratic: 50% of Mobile Phase B; flow rate: 80 mL/min; backpressure=100 bar, column temperature=40° C.]. The chiral purity was determined using SFC analytical method on Chiralcel OD-3 50 mm×4.6 mm×3 μm column; Mobile phase A: CO₂; Mobile phase B: EtOH with 0.2% of NH₃; gradient: 5-40% of Mobile Phase B in 2.0 min and hold 40% for 1.2 min then 5% of Mobile Phase B for 0.8 min; flow rate: 4.0 mL/min; backpressure: 1500 psi; column temperature: 35° C. to provide Example 693: peak 1 ((SFC-MS) m/z (M+H)⁺=656.34 at retention time: 2.201 min, 100% ee) and Example 692: peak 2 ((SFC-MS) m/z (M+H)⁺=656.35 at retention time: 2.705 min, 100% ee).

Example 694 and 695\hat{ }: Example 696 was separated into its component isomers using SFC [Column: DAICEL CHIRALCEL OD 250 mm×30 mm×10 μm; Mobile phase: 40% CO₂/60% EtOH (0.1% NH₄OH); isocratic: 60% of Mobile Phase B; flow rate: 80 mL/min; backpressure=100 bar, column temperature=40° C.]. The chiral purity was determined using SFC analytical method on Chiralcel OD-3 50 mm×4.6 mm×3 μm column; Mobile phase A: CO₂; Mobile phase B: EtOH with 0.2% of NH₃; isocratic: 40% of Mobile Phase B; flow rate: 3.5 mL/min; backpressure: 1500 psi; column temperature: 35° C. to provide Example 695: peak 1 ((SFC-MS) m/z (M+H)⁺=642.35 at retention time: 0.836 min, 100% ee) and Example 694: peak 2 ((SFC-MS) m/z (M+H)⁺=642.32 at retention time: 1.519 min, 100% ee).

Example 702 and 705\hat{ }: Example 703 was separated into its component isomers using SFC [Column: DAICEL CHIRALCEL OD 250 mm×30 mm×10 μm; Mobile phase: 50% CO₂/50% EtOH (0.1% NH₄OH); isocratic: 50% of Mobile Phase B; gradient time=30 min; flow rate: 80 mL/min]. The chiral purity was determined using SFC analytical method on IM-3 50 mm×4.6 mm×3 μm column; Mobile phase A: CO₂; Mobile phase B: EtOH with 0.2% of NH₃; isocratic: 50% of Mobile Phase B; flow rate: 3.5 mL/min; backpressure: 1500 psi; column temperature: 35° C. to provide Example 702: peak 1 ((SFC-MS) m/z (M+H)⁺=655.78 at retention time: 1.217 min, 100% ee) and Example 705: peak 2 ((SFC-MS) m/z (M+H)⁺=655.78 at retention time: 2.492 min, 100% ee).

Example 713 and 714\hat{ }: A mixture was separated into its component isomers using SFC [Column: Pirkle Covalent (R,R) Whelk-01 250 mm×21.1 mm×5 μm; Mobile phase: 40% MeOH (0.2% NH₄OH)/60% ACN; flow rate=75 mL/min, gradient time=70 min, backpressure=120 bar]. The chiral purity was determined using SFC analytical method on Regis (R,R) Whelk-01 50 mm×5 mm×3 μm column; Mobile phase A: MeOH (0.2% NH₄OH); Mobile phase B: ACN; isocratic gradient: 60% of Mobile Phase B; flow rate: 5.0 mL/min; backpressure: 120 bar, gradient time: 30 min to provide Example 714: peak 1 ((SFC-MS) m/z (M/2+H)⁺=308.2 at retention time: 16.09 min) and Example 713: peak 2 ((SFC-MS) m/z (M+H)⁺=615.4 at retention time: 16.43 min).

### Examples V1-V142

The compounds in Table 2 can be prepared using similar chemistry from Examples 1-758 that are commercially available or that can be synthesized by literature methods to form the resultant compounds of the disclosure.

TABLE 2

| Ex. | Structure | ChemDraw Name |
|---|---|---|
| V1 | 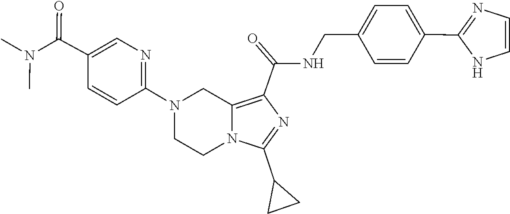 [SMILES: CN(C)C(=O)c1ccc(N2CCn3c(C4CC4)nc(C(=O)NCc4ccc(-c5ncc[nH]5)cc4)c3C2)nc1] | N-(4-(1H-imidazol-2-yl)benzyl)-3- cyclopropyl-7-(5- (dimethylcarbamoyl)pyridin-2-yl)- 5,6,7,8-tetrahydroimidazo[1,5- a]pyrazine-1-carboxamide |
| V2 | 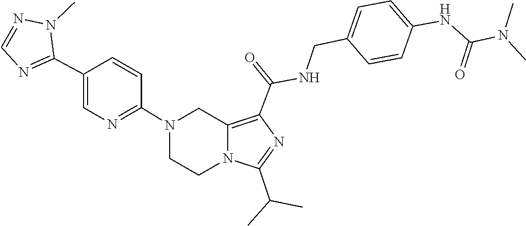 [SMILES: CC(C)c1nc(C(=O)NCc2ccc(NC(=O)N(C)C)cc2)c2n1CCN(c1ccc(-c3ncnn3C)cn1)C2] | N-(4-(3,3-dimethylureido)benzyl)- 3-isopropyl-7-(5-(1-methyl-1H- 1,2,4-triazol-5-yl)pyridin-2-yl)- 5,6,7,8-tetrahydroimidazo[1,5- a]pyrazine-1-carboxamide |
| V3 | 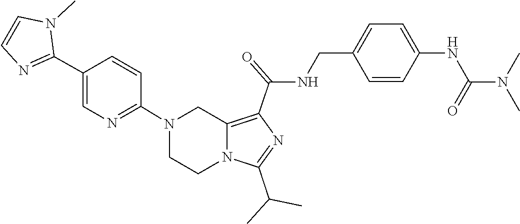 [SMILES: CC(C)c1nc(C(=O)NCc2ccc(NC(=O)N(C)C)cc2)c2n1CCN(c1ccc(-c3nccn3C)cn1)C2] | N-(4-(3,3-dimethylureido)benzyl)- 3-isopropyl-7-(5-(1-methyl-1H- imidazol-2-yl)pyridin-2-yl)-5,6,7,8- tetrahydroimidazo[1,5-a]pyrazine-1- carboxamide |
| V4 | 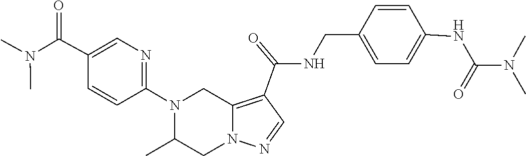 | 5-(5-(dimethylcarbamoyl)pyridin- 2-yl)-N-(4-(3,3- dimethylureido)benzyl)-6-methyl- 4,5,6,7-tetrahydropyrazolo[1,5- a]pyrazine-3-carboxamide |
| V5 | 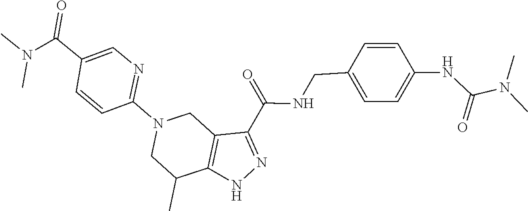 | 5-(5-(dimethylcarbamoyl)pyridin- 2-yl)-N-(4-(3,3- dimethylureido)benzyl)-7-methyl- 4,5,6,7-tetrahydro-1H- pyrazolo[4,3-c]pyridine-3- carboxamide |
| V6 | 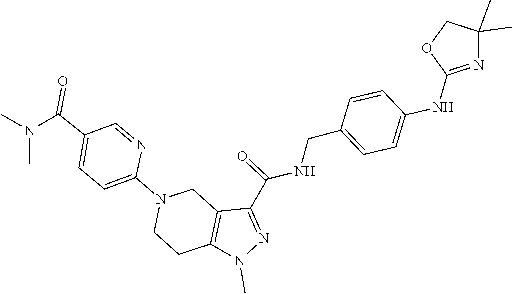 | N-(4-((4,4-dimethyl-4,5- dihydrooxazol-2- yl)amino)benzyl)-5-(5- (dimethylcarbamoyl)pyridin-2-yl)- 1-methyl-4,5,6,7-tetrahydro-1H- pyrazolo[4,3-c]pyridine-3- carboxamide |
| V7 | 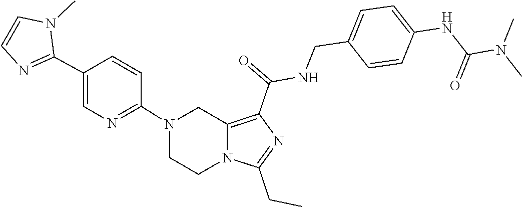 [SMILES: CCc1nc(C(=O)NCc2ccc(NC(=O)N(C)C)cc2)c2n1CCN(c1ccc(-c3nccn3C)cn1)C2] | N-(4-(3,3-dimethylureido)benzyl)- 3-ethyl-7-(5-(1-methyl-1H- imidazol-2-yl)pyridin-2-yl)-5,6,7,8- tetrahydroimidazo[1,5-a]pyrazine- 1-carboxamide |
| V8 | 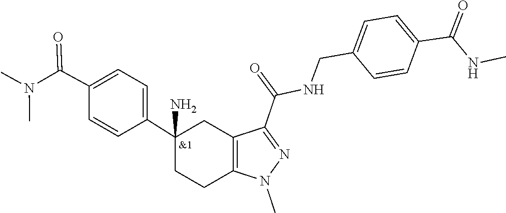 | rac-(R)-5-amino-5-(4- (dimethylcarbamoyl)phenyl)-1- methyl-N-(4- (methylcarbamoyl)benzyl)- 4,5,6,7-tetrahydro-1H-indazole-3- carboxamide |
| V9 | 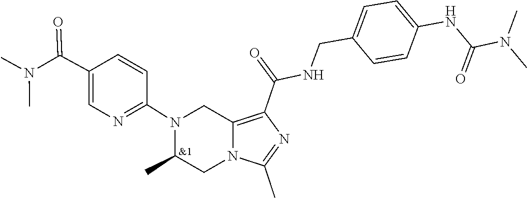 | rac-(R)-7-(5- (dimethylcarbamoyl)pyridin-2-yl)- N-(4-(3,3-dimethylureido)benzyl)- 3,6-dimethyl-5,6,7,8- tetrahydroimidazo[1,5-a]pyrazine- 1-carboxamide |
| V10 | 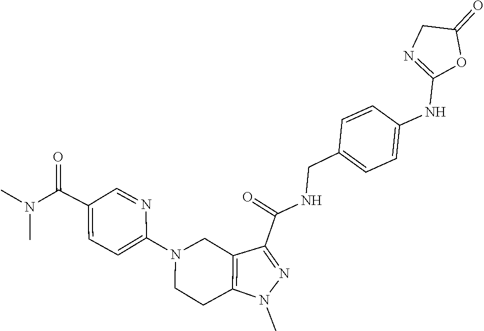 [SMILES: CN(C)C(=O)c1ccc(N2CCc3c(c(C(=O)NCc4ccc(NC5=NCC(=O)O5)cc4)nn3C)C2)nc1] | 5-(5-(dimethylcarbamoyl)pyridin- 2-yl)-1-methyl-N-(4-((5-oxo-4,5- dihydrooxazol-2- yl)amino)benzyl)-4,5,6,7- tetrahydro-1H-pyrazolo[4,3- c]pyridine-3-carboxamide |
| V11 | 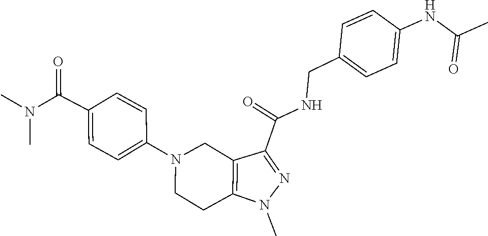 [SMILES: CC(=O)Nc1ccc(CNC(=O)c2nn(C)c3c2CN(c2ccc(C(=O)N(C)C)cc2)CC3)cc1] | N-(4-acetamidobenzyl)-5-(4- (dimethylcarbamoyl)phenyl)-1- methyl-4,5,6,7-tetrahydro-1H- pyrazolo[4,3-c]pyridine-3- carboxamide |
| V12 | 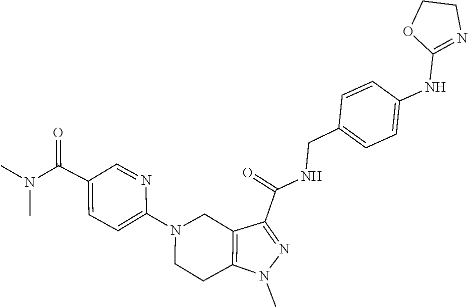 [SMILES: CN(C)C(=O)c1ccc(N2CCc3c(c(C(=O)NCc4ccc(NC5=NCCO5)cc4)nn3C)C2)nc1] | N-(4-((4,5-dihydrooxazol-2- yl)amino)benzyl)-5-(5- (dimethylcarbamoyl)pyridin-2-yl)- 1-methyl-4,5,6,7-tetrahydro-1H- pyrazolo[4,3-c]pyridine-3- carboxamide |
| V13 | 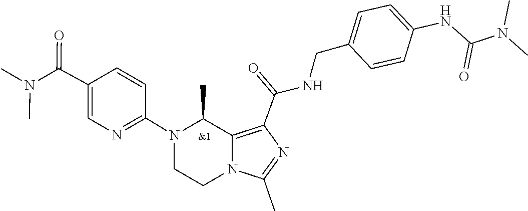 | rac-(R)-7-(5- (dimethylcarbamoyl)pyridin-2-yl)- N-(4-(3,3-dimethylureido)benzyl)- 3,8-dimethyl-5,6,7,8- tetrahydroimidazo[1,5-a]pyrazine- 1-carboxamide |
| V14 | 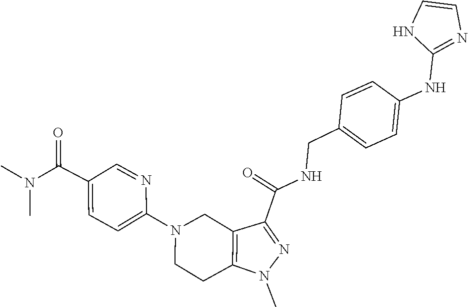 [SMILES: CN(C)C(=O)c1ccc(N2CCc3c(c(C(=O)NCc4ccc(Nc5ncc[nH]5)cc4)nn3C)C2)nc1] | N-(4-((1H-imidazol-2- yl)amino)benzyl)-5-(5- (dimethylcarbamoyl)pyridin-2-yl)- 1-methyl-4,5,6,7-tetrahydro-1H- pyrazolo[4,3-c]pyridine-3- carboxamide |
| V15 | 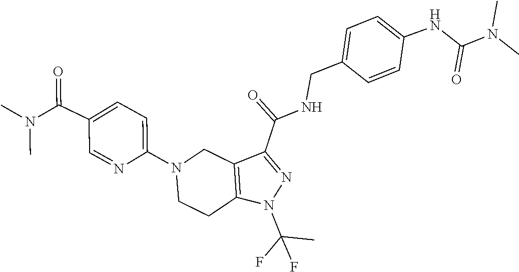 | 1-(1,1-difluoroethyl)-5-(5- (dimethylcarbamoyl)pyridin-2-yl)- N-(4-(3,3-dimethylureido)benzyl)- 4,5,6,7-tetrahydro-1H- pyrazolo[4,3-c]pyridine-3- carboxamide |
| V16 | 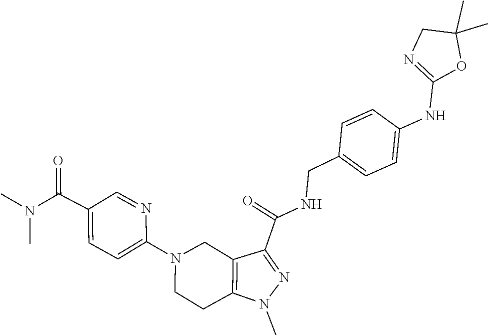 | N-(4-((5,5-dimethyl-4,5- dihydrooxazol-2- yl)amino)benzyl)-5-(5- (dimethylcarbamoyl)pyridin-2-yl)- 1-methyl-4,5,6,7-tetrahydro-1H- pyrazolo[4,3-c]pyridine-3- carboxamide |
| V17 | 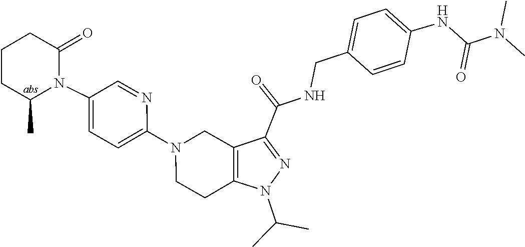 [SMILES: CC(C)n1nc(C(=O)NCc2ccc(NC(=O)N(C)C)cc2)c2c1CCN(c1ccc(N3C(=O)CCC[C@@H]3C)cn1)C2] | (S)-N-(4-(3,3- dimethylureido)benzyl)-1- isopropyl-5-(5-(2-methyl-6- oxopiperidin-1-yl)pyridin-2-yl)- 4,5,6,7-tetrahydro-1H- pyrazolo[4,3-c]pyridine-3- carboxamide |
| V18 | 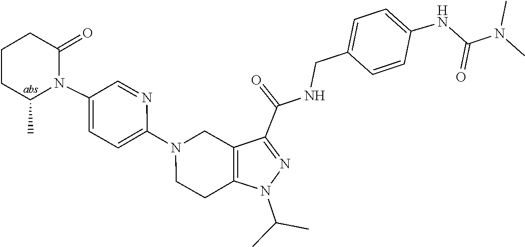 [SMILES: CC(C)n1nc(C(=O)NCc2ccc(NC(=O)N(C)C)cc2)c2c1CCN(c1ccc(N3C(=O)CCC[C@H]3C)cn1)C2] | (R)-N-(4-(3,3- dimethylureido)benzyl)-1- isopropyl-5-(5-(2-methyl-6- oxopiperidin-1-yl)pyridin-2-yl)- 4,5,6,7-tetrahydro-1H- pyrazolo[4,3-c]pyridine-3- carboxamide |
| V19 | 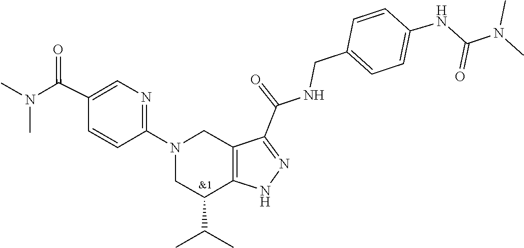 | rac-(R)-5-(5- (dimethylcarbamoyl)pyridin-2-yl)- N-(4-(3,3-dimethylureido)benzyl)- 7-isopropyl-4,5,6,7-tetrahydro- 1H-pyrazolo[4,3-c]pyridine-3- carboxamide |
| V20 | 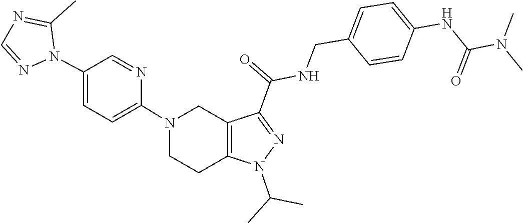 [SMILES: Cc1ncnn1-c1ccc(N2CCc3c(c(C(=O)NCc4ccc(NC(=O)N(C)C)cc4)nn3C(C)C)C2)nc1] | N-(4-(3,3-dimethylureido)benzyl)- 1-isopropyl-5-(5-(5-methyl-1H- 1,2,4-triazol-1-yl)pyridin-2-yl)- 4,5,6,7-tetrahydro-1H- pyrazolo[4,3-c]pyridine-3- carboxamide |
| V21 | 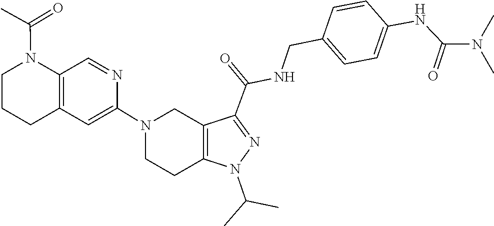 [SMILES: CC(=O)N1CCCc2cc(N3CCc4c(c(C(=O)NCc5ccc(NC(=O)N(C)C)cc5)nn4C(C)C)C3)ncc21] | 5-(1-acetyl-1,2,3,4-tetrahydro- 1,7-naphthyridin-6-yl)-N-(4-(3,3- dimethylureido)benzyl)-1- isopropyl-4,5,6,7-tetrahydro-1H- pyrazolo[4,3-c]pyridine-3- carboxamide |
| V22 | 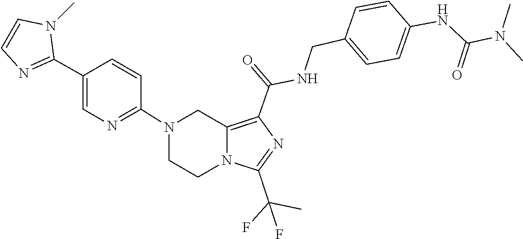 [SMILES: CN(C)C(=O)Nc1ccc(CNC(=O)c2nc(C(C)(F)F)n3c2CN(c2ccc(-c4nccn4C)cn2)CC3)cc1] | 3-(1,1-difluoroethyl)-N-(4-(3,3- dimethylureido)benzyl)-7-(5-(1- methyl-1H-imidazol-2-yl)pyridin- 2-yl)-5,6,7,8- tetrahydroimidazo[1,5-a]pyrazine- 1-carboxamide |
| V23 | 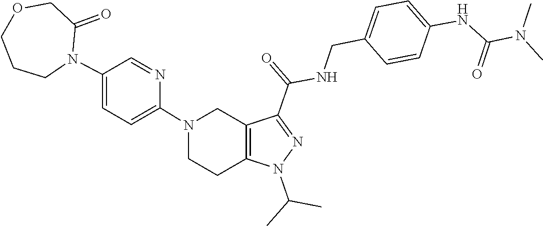 [SMILES: CC(C)n1nc(C(=O)NCc2ccc(NC(=O)N(C)C)cc2)c2c1CCN(c1ccc(N3CCCOCC3=O)cn1)C2] | N-(4-(3,3-dimethylureido)benzyl)- 1-isopropyl-5-(5-(3-oxo-1,4- oxazepan-4-yl)pyridin-2-yl)- 4,5,6,7-tetrahydro-1H- pyrazolo[4,3-c]pyridine-3- carboxamide |
| V24 | 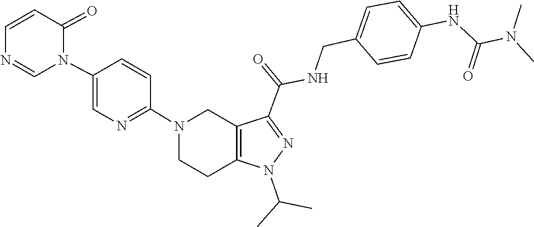 [SMILES: CC(C)n1nc(C(=O)NCc2ccc(NC(=O)N(C)C)cc2)c2c1CCN(c1ccc(-n3cnccc3=O)cn1)C2] | N-(4-(3,3-dimethylureido)benzyl)- 1-isopropyl-5-(5-(6-oxopyrimidin- 1(6H)-yl)pyridin-2-yl)-4,5,6,7-tetrahydro- 1H-pyrazolo[4,3-c]pyridine-3- carboxamide |
| V25 | 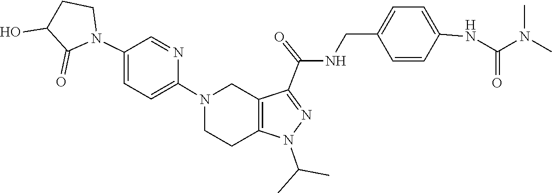 [SMILES: CC(C)n1nc(C(=O)NCc2ccc(NC(=O)N(C)C)cc2)c2c1CCN(c1ccc(N3CCC(O)C3=O)cn1)C2] | N-(4-(3,3-dimethylureido)benzyl)- 5-(5-(3-hydroxy-2-oxopyrrolidin-1- yl)pyridin-2-yl)-1-isopropyl- 4,5,6,7-tetrahydro-1H- pyrazolo[4,3-c]pyridine-3- carboxamide |
| V26 | 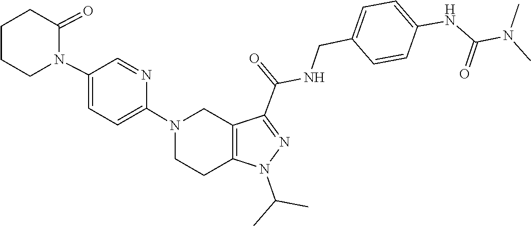 [SMILES: CC(C)n1nc(C(=O)NCc2ccc(NC(=O)N(C)C)cc2)c2c1CCN(c1ccc(N3CCCCC3=O)cn1)C2] | N-(4-(3,3-dimethylureido)benzyl)- 1-isopropyl-5-(5-(2-oxopiperidin- 1-yl)pyridin-2-yl)-4,5,6,7-tetrahydro- 1H-pyrazolo[4,3-c]pyridine-3- carboxamide |
| V27 | 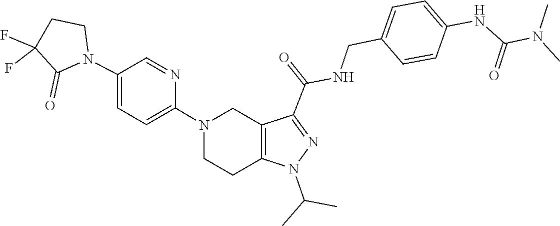 [SMILES: CC(C)n1nc(C(=O)NCc2ccc(NC(=O)N(C)C)cc2)c2c1CCN(c1ccc(N3CCC(F)(F)C3=O)cn1)C2] | 5-(5-(3,3-difluoro-2-oxopyrrolidin- 1-yl)pyridin-2-yl)-N-(4-(3,3- dimethylureido)benzyl)-1- isopropyl-4,5,6,7-tetrahydro-1H- pyrazolo[4,3-c]pyridine-3- carboxamide |
| V28 | 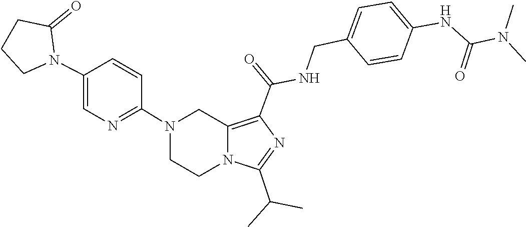 [SMILES: CC(C)c1nc(C(=O)NCc2ccc(NC(=O)N(C)C)cc2)c2n1CCN(c1ccc(N3CCCC3=O)cn1)C2] | N-(4-(3,3-dimethylureido)benzyl)- 3-isopropyl-7-(5-(2-oxopyrrolidin- 1-yl)pyridin-2-yl)-5,6,7,8- tetrahydroimidazo[1,5-a]pyrazine- 1-carboxamide |
| V29 | 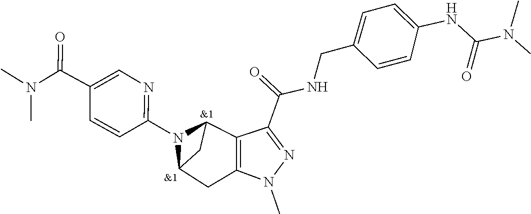 | rac-(4R,6S)-8-(5- (dimethylcarbamoyl)pyridin-2-yl)- N-(4-(3,3-dimethylureido)benzyl)- 1-methyl-4,5,6,7-tetrahydro-1H- 4,6-epiminoindazole-3- carboxamide |
| V30 | 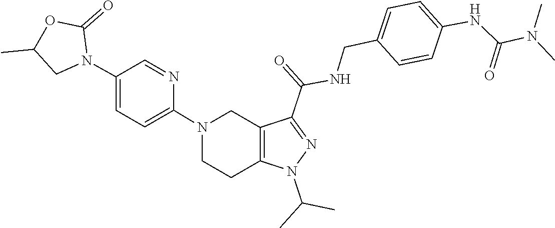 | N-(4-(3,3-dimethylureido)benzyl)- 1-isopropyl-5-(5-(5-methyl-2- oxooxazolidin-3-yl)pyridin-2-yl)- 4,5,6,7-tetrahydro-1H- pyrazolo[4,3-c]pyridine-3- carboxamide |
| V31 | 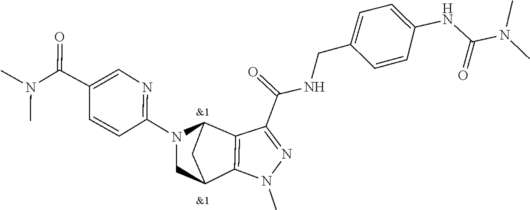 | rac-(4R,7R)-5-(5- (dimethylcarbamoyl)pyridin-2-yl)- N-(4-(3,3-dimethylureido)benzyl)- 1-methyl-4,5,6,7-tetrahydro-1H- 4,7-methanopyrazolo[4,3- c]pyridine-3-carboxamide |
| V32 | 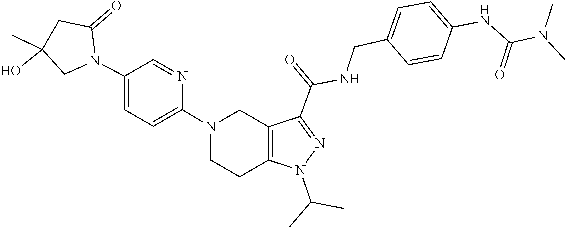 | N-(4-(3,3-dimethylureido)benzyl)- 5-(5-(4-hydroxy-4-methyl-2- oxopyrrolidin-1-yl)pyridin-2-yl)-1- isopropyl-4,5,6,7-tetrahydro-1H- pyrazolo[4,3-c]pyridine-3- carboxamide |
| V33 | 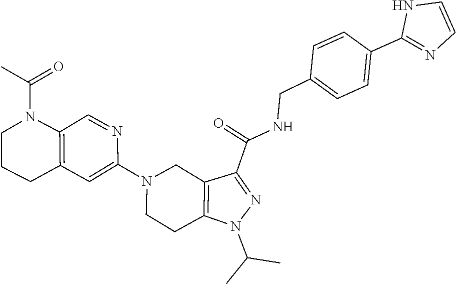 [SMILES: CC(=O)N1CCCc2cc(N3CCc4c(c(C(=O)NCc5ccc(-c6ncc[nH]6)cc5)nn4C(C)C)C3)ncc21] | N-(4-(1H-imidazol-2-yl)benzyl)-5- (1-acetyl-1,2,3,4-tetrahydro-1,7- naphthyridin-6-yl)-1-isopropyl- 4,5,6,7-tetrahydro-1H- pyrazolo[4,3-c]pyridine-3- carboxamide |
| V34 | 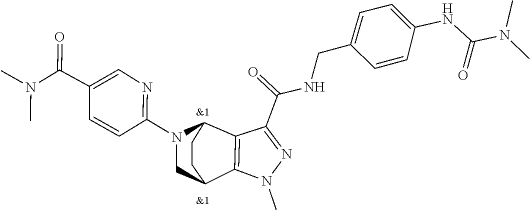 | rac-(4R,7R)-9-(5- (dimethylcarbamoyl)pyridin-2-yl)- N-(4-(3,3-dimethylureido)benzyl)- 1-methyl-4,5,6,7-tetrahydro-1H- 4,7-(epiminomethano)indazole-3- carboxamide |
| V35 | 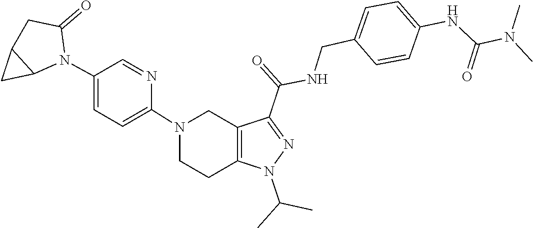 [SMILES: CC(C)n1nc(C(=O)NCc2ccc(NC(=O)N(C)C)cc2)c2c1CCN(c1ccc(N3C(=O)CC4CC43)cn1)C2] | N-(4-(3,3-dimethylureido)benzyl)- 1-isopropyl-5-(5-(3-oxo-2- azabicyclo[3.1.0]hexan-2- yl)pyridin-2-yl)-4,5,6,7-tetrahydro- 1H-pyrazolo[4,3-c]pyridine-3- carboxamide |
| V36 | 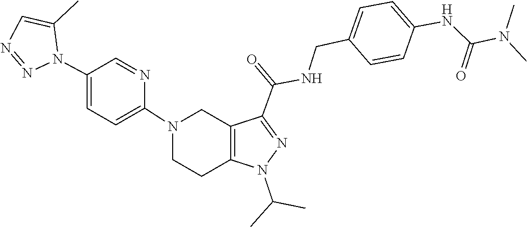 [SMILES: Cc1cnnn1-c1ccc(N2CCc3c(c(C(=O)NCc4ccc(NC(=O)N(C)C)cc4)nn3C(C)C)C2)nc1] | N-(4-(3,3-dimethylureido)benzyl)- 1-isopropyl-5-(5-(5-methyl-1H- 1,2,3-triazol-1-yl)pyridin-2-yl)- 4,5,6,7-tetrahydro-1H- pyrazolo[4,3-c]pyridine-3- carboxamide |
| V37 | 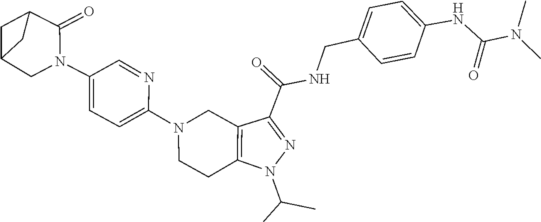 [SMILES: CC(C)n1nc(C(=O)NCc2ccc(NC(=O)N(C)C)cc2)c2c1CCN(c1ccc(N3CC4CC(C4)C3=O)cn1)C2] | N-(4-(3,3-dimethylureido)benzyl)- 1-isopropyl-5-(5-(2-oxo-3- azabicyclo[3.1.1]heptan-3- yl)pyridin-2-yl)-4,5,6,7-tetrahydro-1H- pyrazolo[4,3-c]pyridine-3- carboxamide |
| V38 | 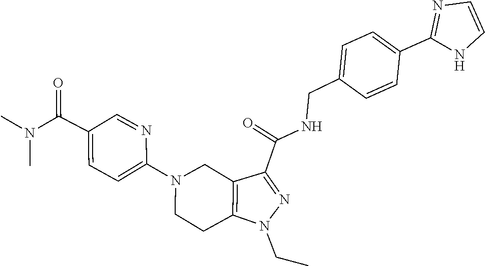 [SMILES: CCn1nc(C(=O)NCc2ccc(-c3ncc[nH]3)cc2)c2c1CCN(c1ccc(C(=O)N(C)C)cn1)C2] | N-(4-(1H-imidazol-2-yl)benzyl)-5- (5-(dimethylcarbamoyl)pyrimidin- 2-yl)-1-ethyl-4,5,6,7-tetrahydro-1H- pyrazolo[4,3-c]pyridine-3- carboxamide |
| V39 | 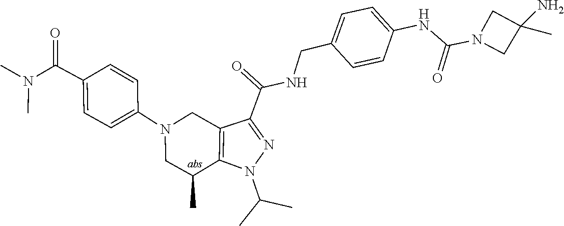 | (S)-N-(4-(3-amino-3- methylazetidine-1- carboxamido)benzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V40 | 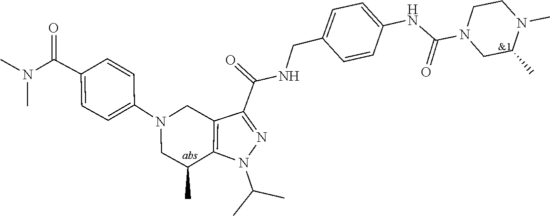 | (S)-5-(4- (dimethylcarbamoyl)phenyl)-N-(4- ((RS)-3,4-dimethylpiperazine-1- carboxamido)benzyl)-1-isopropyl- 7-methyl-4,5,6,7-tetrahydro-1H- pyrazolo[4,3-c]pyridine-3- carboxamide |
| V41 | 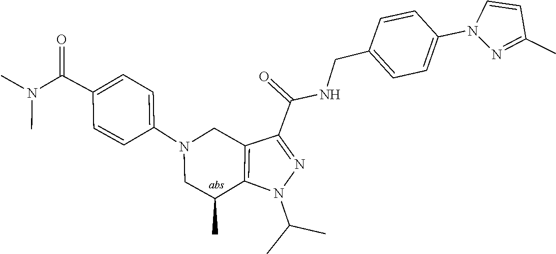 | (S)-5-(4- (dimethylcarbamoyl)phenyl)-1- isopropyl-7-methyl-N-(4-(3- methyl-1H-pyrazol-1-yl)benzyl)- 4,5,6,7-tetrahydro-1H- pyrazolo[4,3-c]pyridine-3- carboxamide |
| V42 | 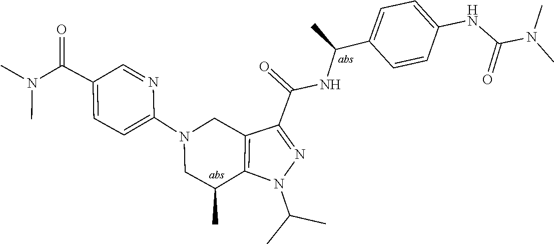 | (S)-5-(5- (dimethylcarbamoyl)pyridin-2-yl)- N-((S)-1-(4-(3,3- dimethylureido)phenyl)ethyl)-1- isopropyl-7-methyl- 4,5,6,7-tetrahydro-1H- pyrazolo[4,3-c]pyridine-3- carboxamide |
| V43 | 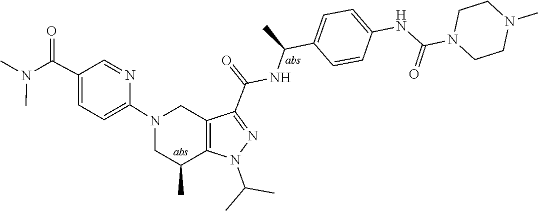 | (S)-5-(5- (dimethylcarbamoyl)pyridin-2-yl)- 1-isopropyl-7-methyl-N-((S)-1-(4- (4-methylpiperazine-1- carboxamido)phenyl)ethyl)- 4,5,6,7-tetrahydro-1H- pyrazolo[4,3-c]pyridine-3- carboxamide |
| V44 | 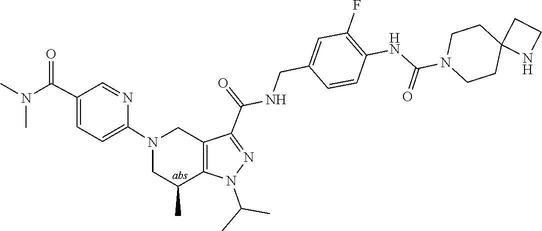 [SMILES: CC(C)n1nc(C(=O)NCc2ccc(NC(=O)N3CCC4(CCN4)CC3)c(F)c2)c2c1[C@@H](C)CN(c1ccc(C(=O)N(C)C)cn1)C2] | (S)-5-(4- (dimethylcarbamoyl)phenyl)-N-(3- fluoro-4-(1,7- diazaspiro[3.5]nonane-7- carboxamido)benzyl)-1-isopropyl- 7-methyl-4,5,6,7-tetrahydro-1H- pyrazolo[4,3-c]pyridine-3- carboxamide |
| V45 | 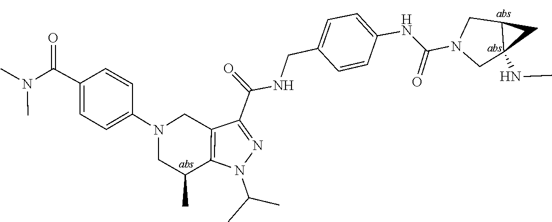 | (S)-5-(4- (dimethylcarbamoyl)phenyl)-1- isopropyl-7-methyl-N-(4-((1S,5R)- 1-(methylamino)-3- azabicyclo[3.1.0]hexane-3- carboxamido)benzyl)- 4,5,6,7-tetrahydro-1H- pyrazolo[4,3-c]pyridine-3- carboxamide |
| V46 | 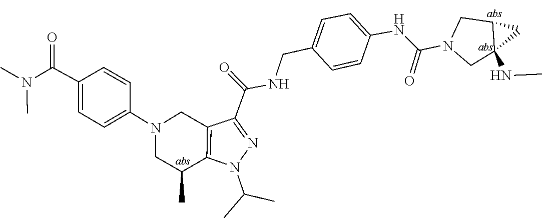 | (S)-5-(4- (dimethylcarbamoyl)phenyl)-1- isopropyl-7-methyl-N-(4-((1R,5S)- 1-(methylamino)-3- azabicyclo[3.1.0]hexane-3- carboxamido)benzyl)- 4,5,6,7-tetrahydro-1H- pyrazolo[4,3-c]pyridine-3- carboxamide |
| V47 | 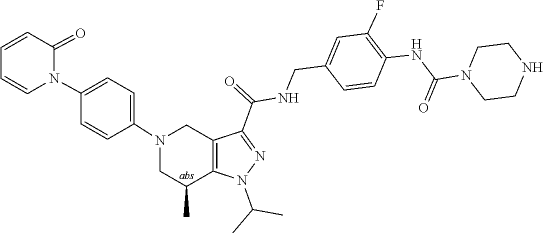 [SMILES: CC(C)n1nc(C(=O)NCc2ccc(NC(=O)N3CCNCC3)c(F)c2)c2c1[C@@H](C)CN(c1ccc(-n3ccccc3=O)cc1)C2] | (S)-N-(3-fluoro-4-(piperazine-1- carboxamido)benzyl)-1-isopropyl- 7-methyl-5-(4-(2-oxopyridin- 1(2H)-yl)phenyl)- 4,5,6,7-tetrahydro-1H- pyrazolo[4,3-c]pyridine-3- carboxamide |
| V48 | 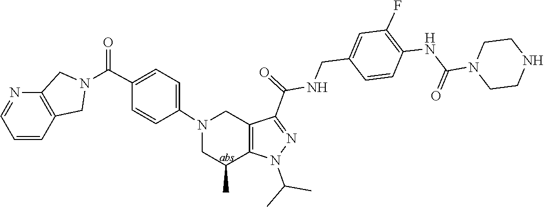 [SMILES: CC(C)n1nc(C(=O)NCc2ccc(NC(=O)N3CCNCC3)c(F)c2)c2c1[C@@H](C)CN(c1ccc(C(=O)N3Cc4cccnc4C3)cc1)C2] | (S)-5-(4-(6,7-dihydro-5H- pyrrolo[3,4-b]pyridine-6- carbonyl)phenyl)-N-(3-fluoro-4- (piperazine-1- carboxamido)benzyl)-1-isopropyl- 7-methyl-4,5,6,7-tetrahydro-1H- pyrazolo[4,3-c]pyridine-3- carboxamide |
| V49 | 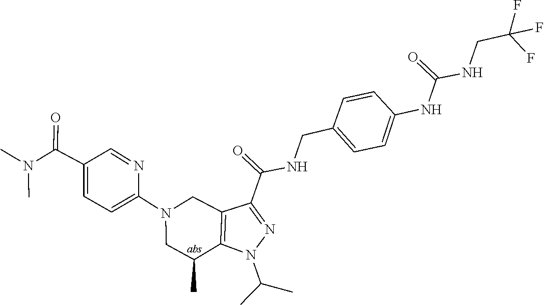 [SMILES: CC(C)n1nc(C(=O)NCc2ccc(NC(=O)NCC(F)(F)F)cc2)c2c1[C@@H](C)CN(c1ccc(C(=O)N(C)C)cn1)C2] | (S)-5-(5- (dimethylcarbamoyl)pyridin-2-yl)- 1-isopropyl-7-methyl-N-(4-(3- (2,2,2- trifluoroethyl)ureido)benzyl)- 4,5,6,7-tetrahydro-1H- pyrazolo[4,3-c]pyridine-3- carboxamide |
| V50 | 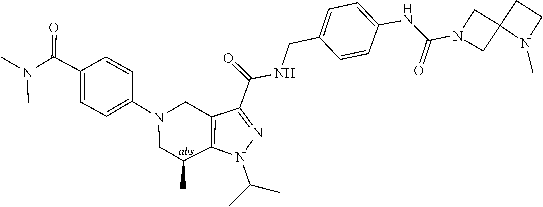 [SMILES: CC(C)n1nc(C(=O)NCc2ccc(NC(=O)N3CC4(CCN4C)C3)cc2)c2c1[C@@H](C)CN(c1ccc(C(=O)N(C)C)cc1)C2] | (S)-5-(4- (dimethylcarbamoyl)phenyl)-1- isopropyl-7-methyl-N-(4-(1- methyl-1,6- diazaspiro[3.3]heptane-6- carboxamido)benzyl)- 4,5,6,7-tetrahydro-1H- pyrazolo[4,3-c]pyridine-3- carboxamide |
| V51 | 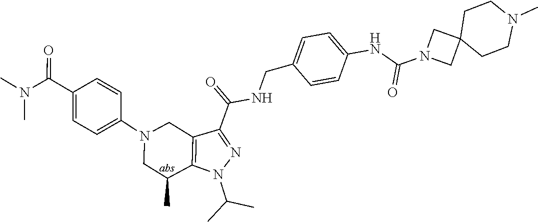 | (S)-5-(4- (dimethylcarbamoyl)phenyl)-1- isopropyl-7-methyl-N-(4-(7- methyl-2,7- diazaspiro[3.5]nonane-2- carboxamido)benzyl)- 4,5,6,7-tetrahydro-1H- pyrazolo[4,3-c]pyridine-3- carboxamide |
| V52 | 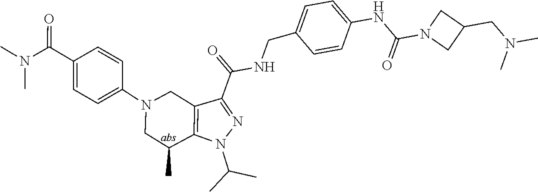 | (S)-N-(4-(3- ((dimethylamino)methyl)azetidine- 1-carboxamido)benzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V53 | 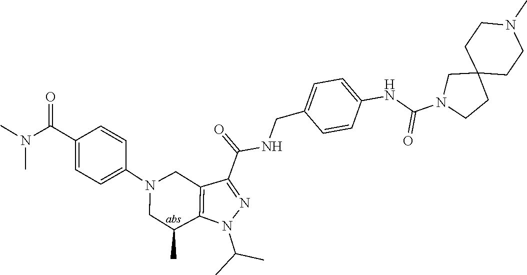 | (S)-N-(4-((5-(4- (dimethylcarbamoyl)phenyl)-1- isopropyl-7-methyl-4,5,6,7-tetrahydro- 1H-pyrazolo[4,3-c]pyridine-3- carboxamido)methyl)phenyl)-8- methyl-2,8- diazaspiro[4.5]decane-2- carboxamide |
| V54 | 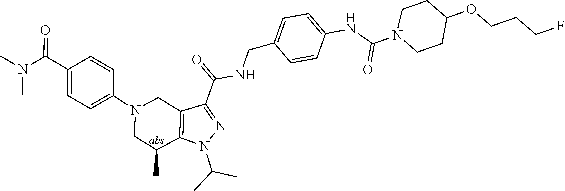 [SMILES: CC(C)n1nc(C(=O)NCc2ccc(NC(=O)N3CCC(OCCCF)CC3)cc2)c2c1[C@@H](C)CN(c1ccc(C(=O)N(C)C)cc1)C2] | (S)-5-(4- (dimethylcarbamoyl)phenyl)-N-(4- (4-(3-fluoropropoxy)piperidine-1- carboxamido)benzyl)-1-isopropyl- 7-methyl-4,5,6,7-tetrahydro-1H- pyrazolo[4,3-c]pyridine-3- carboxamide |
| V55 | 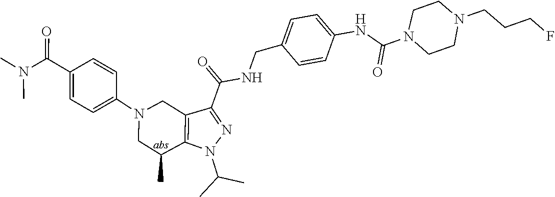 [SMILES: CC(C)n1nc(C(=O)NCc2ccc(NC(=O)N3CCN(CCCF)CC3)cc2)c2c1[C@@H](C)CN(c1ccc(C(=O)N(C)C)cc1)C2] | (S)-5-(4- (dimethylcarbamoyl)phenyl)-N-(4- (4-(3-fluoropropyl)piperazine-1- carboxamido)benzyl)-1-isopropyl- 7-methyl-4,5,6,7-tetrahydro-1H- pyrazolo[4,3-c]pyridine-3- carboxamide |
| V56 | 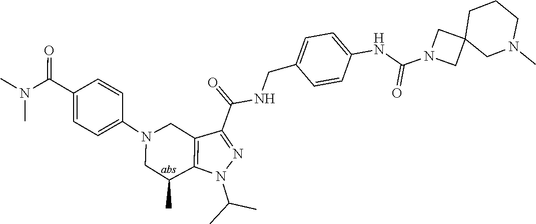 | (S)-N-(4-((5-(4- (dimethylcarbamoyl)phenyl)-1- isopropyl-7-methyl- 4,5,6,7-tetrahydro-1H- pyrazolo[4,3-c]pyridine-3- carboxamido)methyl)phenyl)-8- methyl-5-oxa-2,8- diazaspiro[3.5]nonane-2- carboxamide |
| V57 | 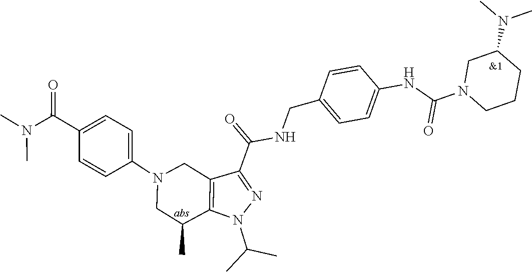 | (S)-N-(4-((RS)-3- (dimethylamino)piperidine-1- carboxamido)benzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V58 | 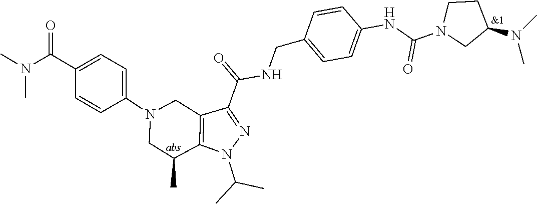 | (S)-N-(4-((RS)-3- (dimethylamino)pyrrolidine-1- carboxamido)benzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V59 | 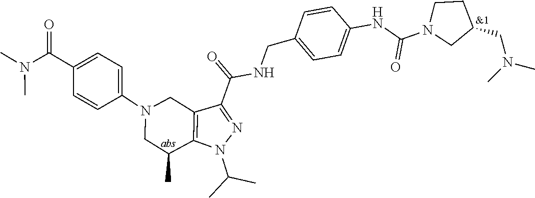 | (S)-N-(4-((RS)-3- ((dimethylamino)methyl)pyrrolidine- 1-carboxamido)benzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V60 | 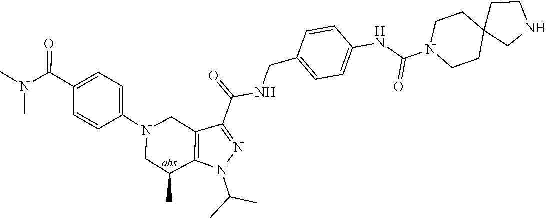 [SMILES: CC(C)n1nc(C(=O)NCc2ccc(NC(=O)N3CCC4(CCNC4)CC3)cc2)c2c1[C@@H](C)CN(c1ccc(C(=O)N(C)C)cc1)C2] | (S)-N-(4-((5-(4- (dimethylcarbamoyl)phenyl)-1- isopropyl-7-methyl- 4,5,6,7-tetrahydro-1H- pyrazolo[4,3-c]pyridine-3- carboxamido)methyl)phenyl)-2,8- diazaspiro[4.5]decane-8- carboxamide |
| V61 | 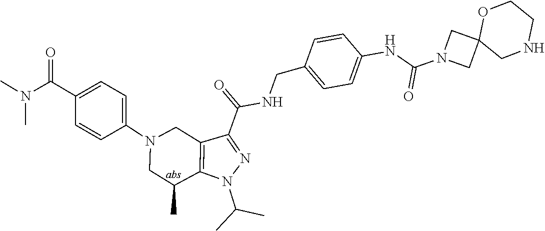 [SMILES: CC(C)n1nc(C(=O)NCc2ccc(NC(=O)N3CC4(CNCCO4)C3)cc2)c2c1[C@@H](C)CN(c1ccc(C(=O)N(C)C)cc1)C2] | (S)-N-(4-((5-(4- (dimethylcarbamoyl)phenyl)-1- isopropyl-7-methyl- 4,5,6,7-tetrahydro-1H- pyrazolo[4,3-c]pyridine-3- carboxamido)methyl)phenyl)-5- oxa-2,8-diazaspiro[3.5]nonane-2- carboxamide |
| V62 | 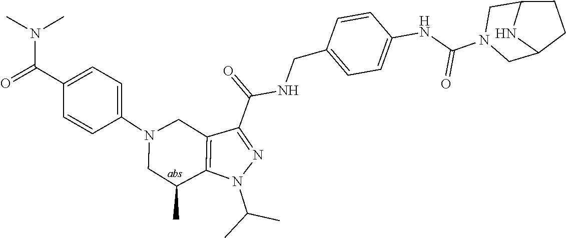 [SMILES: CC(C)n1nc(C(=O)NCc2ccc(NC(=O)N3CC4CCC(C3)N4)cc2)c2c1[C@@H](C)CN(c1ccc(C(=O)N(C)C)cc1)C2] | (7S)-N-(4-(3,8- diazabicyclo[3.2.1]octane-3- carboxamido)benzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V63 | 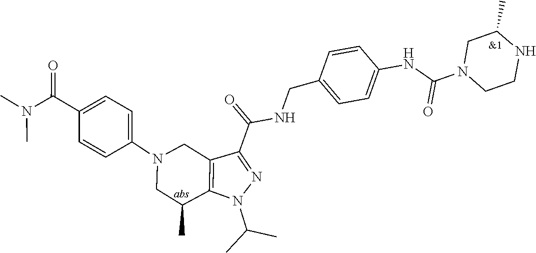 [SMILES: CC(C)n1nc(C(=O)NCc2ccc(NC(=O)N3CCN[C@@H](C)C3)cc2)c2c1[C@@H](C)CN(c1ccc(C(=O)N(C)C)cc1)C2] | (S)-5-(4- (dimethylcarbamoyl)phenyl)-1- isopropyl-7-methyl-N-(4-((RS)-3- methylpiperazine-1- carboxamido)benzyl)- 4,5,6,7-tetrahydro-1H- pyrazolo[4,3-c]pyridine-3- carboxamide |
| V64 | 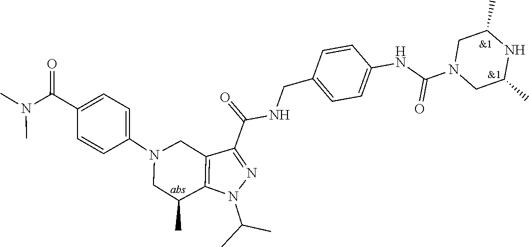 | (S)-5-(4- (dimethylcarbamoyl)phenyl)-N-(4- ((3RS,5SR)-3,5- dimethylpiperazine-1- carboxamido)benzyl)-1-isopropyl- 7-methyl- 4,5,6,7-tetrahydro-1H- pyrazolo[4,3-c]pyridine-3- carboxamide |
| V65 | 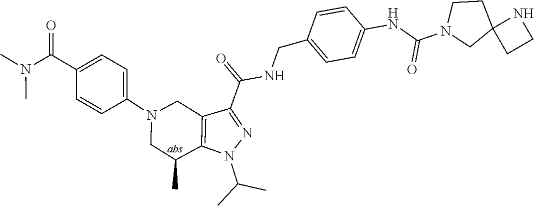 [SMILES: CC(C)n1nc(C(=O)NCc2ccc(NC(=O)N3CCC4(CCN4)C3)cc2)c2c1[C@@H](C)CN(c1ccc(C(=O)N(C)C)cc1)C2] | (7S)-N-(4-(1,6- diazaspiro[3.4]octane-6- carboxamido)benzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V66 | 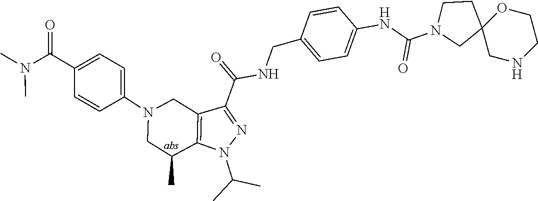 [SMILES: CC(C)n1nc(C(=O)NCc2ccc(NC(=O)N3CCC4(CNCCO4)C3)cc2)c2c1[C@@H](C)CN(c1ccc(C(=O)N(C)C)cc1)C2] | N-(4-(((S)-5-(4- (dimethylcarbamoyl)phenyl)-1- isopropyl-7-methyl- 4,5,6,7-tetrahydro-1H- pyrazolo[4,3-c]pyridine-3- carboxamido)methyl)phenyl)-6- oxa-2,9-diazaspiro[4.5]decane-2- carboxamide |
| V67 | 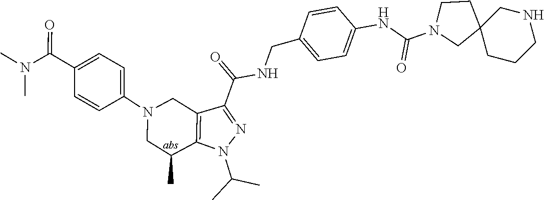 [SMILES: CC(C)n1nc(C(=O)NCc2ccc(NC(=O)N3CCC4(CCCNC4)C3)cc2)c2c1[C@@H](C)CN(c1ccc(C(=O)N(C)C)cc1)C2] | N-(4-(((S)-5-(4- (dimethylcarbamoyl)phenyl)-1- isopropyl-7-methyl- 4,5,6,7-tetrahydro-1H- pyrazolo[4,3-c]pyridine-3- carboxamido)methyl)phenyl)-2,7- diazaspiro[4.5]decane-2- carboxamide |
| V68 | 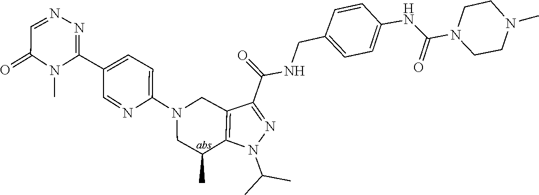 [SMILES: CC(C)n1nc(C(=O)NCc2ccc(NC(=O)N3CCN(C)CC3)cc2)c2c1[C@@H](C)CN(c1ccc(-c3nncc(=O)n3C)cn1)C2] | (S)-1-isopropyl-7-methyl-5-(5-(4- methyl5-oxo-4,5-dihydro-1,2,4- triazin-3-yl)pyridin-2-yl)-N-(4-(4- methylpiperazine-1- carboxamido)benzyl)- 4,5,6,7-tetrahydro-1H- pyrazolo[4,3-c]pyridine-3- carboxamide |
| V69 | 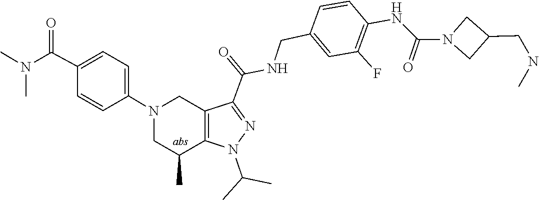 | (S)-N-(4-(3- ((dimethylamino)methyl)azetidine- 1-carboxamido)-3-fluorobenzyl)- 5-(4-(dimethylcarbamoyl)phenyl)- 1-isopropyl-7-methyl- 4,5,6,7-tetrahydro-1H- pyrazolo[4,3-c]pyridine-3- carboxamide |
| V70 | 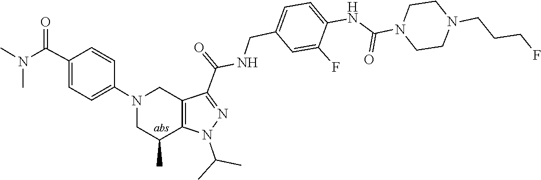 [SMILES: CC(C)n1nc(C(=O)NCc2ccc(NC(=O)N3CCN(CCCF)CC3)c(F)c2)c2c1[C@@H](C)CN(c1ccc(C(=O)N(C)C)cc1)C2] | (S)-5-(4- (dimethylcarbamoyl)phenyl)-N-(3- fluoro-4-(4-(3- fluoropropyl)piperazine-1- carboxamido)benzyl)-1-isopropyl- 7-methyl- 4,5,6,7-tetrahydro-1H- pyrazolo[4,3-c]pyridine-3- carboxamide |
| V71 | 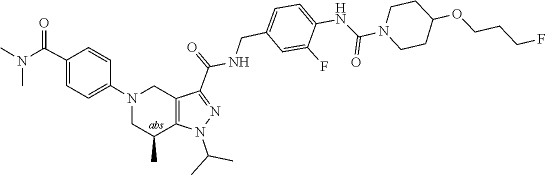 [SMILES: CC(C)n1nc(C(=O)NCc2ccc(NC(=O)N3CCC(OCCCF)CC3)c(F)c2)c2c1[C@@H](C)CN(c1ccc(C(=O)N(C)C)cc1)C2] | (S)-5-(4- (dimethylcarbamoyl)phenyl)-N-(3- fluoro-4-(4-(3- fluoropropoxy)piperidine-1- carboxamido)benzyl)-1-isopropyl- 7-methyl-4,5,6,7-tetrahydro-1H- pyrazolo[4,3-c]pyridine-3- carboxamide |
| V72 | 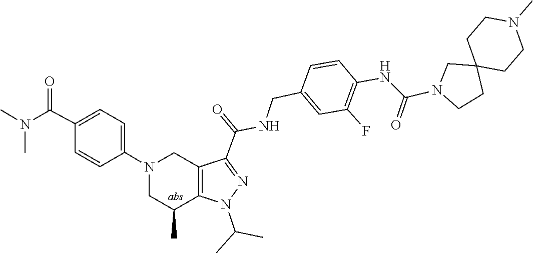 | (S)-N-(4-((5-(4- (dimethylcarbamoyl)phenyl)-1- isopropyl-7-methyl-4,5,6,7-tetrahydro- 1H-pyrazolo[4,3-c]pyridine-3- carboxamido)methyl)-2- fluorophenyl)-8-methyl-2,8- diazaspiro[4.5]decane-2- carboxamide |
| V73 | 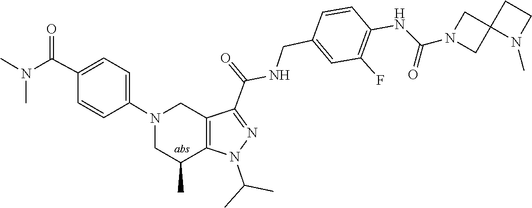 [SMILES: CC(C)n1nc(C(=O)NCc2ccc(NC(=O)N3CC4(CCN4C)C3)c(F)c2)c2c1[C@@H](C)CN(c1ccc(C(=O)N(C)C)cc1)C2] | (S)-5-(4- (dimethylcarbamoyl)phenyl)-N-(3- fluoro-4-(1-methyl-1,6- diazaspiro[3.3]heptane-6- carboxamido)benzyl)-1-isopropyl- 7-methyl-4,5,6,7-tetrahydro-1H- pyrazolo[4,3-c]pyridine-3- carboxamide |
| V74 | 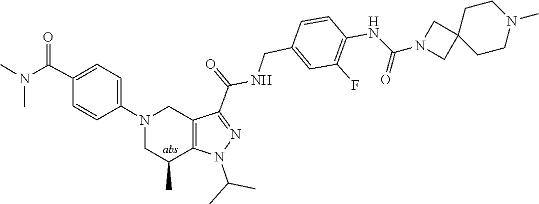 | (S)-5-(4- (dimethylcarbamoyl)phenyl)-N-(3- fluoro-4-(7-methyl-2,7- diazaspiro[3.5]nonane-2- carboxamido)benzyl)-1-isopropyl- 7-methyl-4,5,6,7-tetrahydro-1H- pyrazolo[4,3-c]pyridine-3- carboxamide |
| V75 | 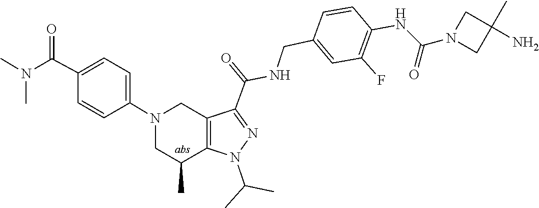 [SMILES: CC(C)n1nc(C(=O)NCc2ccc(NC(=O)N3CC(C)(N)C3)c(F)c2)c2c1[C@@H](C)CN(c1ccc(C(=O)N(C)C)cc1)C2] | (S)-N-(4-(3-amino-3- methylazetidine-1-carboxamido)- 3-fluorobenzyl)-5-(4- (dimethylcarbamoyl)phenyl)-1- isopropyl-7-methyl-4,5,6,7-tetrahydro- 1H-pyrazolo[4,3-c]pyridine-3- carboxamide |
| V76 | 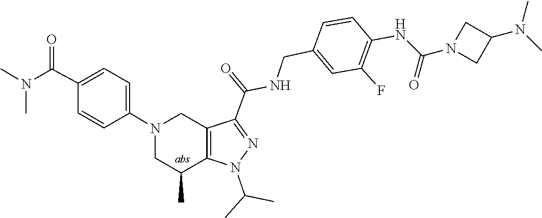 | (S)-N-(4-(3- (dimethylamino)azetidine-1- carboxamido)-3-fluorobenzyl)-5- (4-(dimethylcarbamoyl)phenyl)-1- isopropyl-7-methyl- 4,5,6,7-tetrahydro-1H- pyrazolo[4,3-c]pyridine-3- carboxamide |
| V77 | 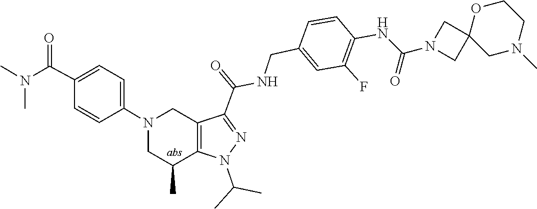 | (S)-N-(4-((5-(4- (dimethylcarbamoyl)phenyl)-1- isopropyl-7-methyl- 4,5,6,7-tetrahydro-1H- pyrazolo[4,3-c]pyridine-3- carboxamido)methyl)-2- fluorophenyl)-8-methyl-5-oxa-2,8- diazaspiro[3.5]nonane-2- carboxamide |
| V78 | 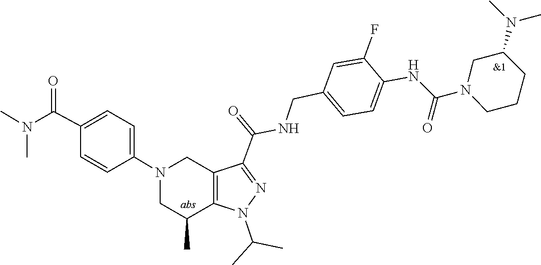 | (S)-N-(4-((RS)-3- (dimethylamino)piperidine-1- carboxamido)-3-fluorobenzyl)-5- (4-(dimethylcarbamoyl)phenyl)-1- isopropyl-7-methyl- 4,5,6,7-tetrahydro-1H- pyrazolo[4,3-c]pyridine-3- carboxamide |
| V79 | 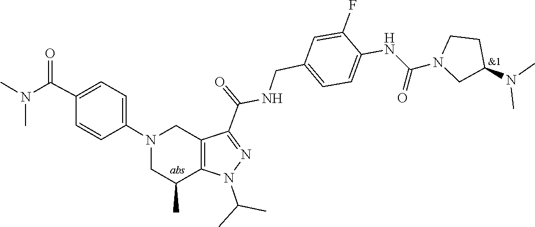 | (S)-N-(4-((RS)-3- (dimethylamino)pyrrolidine-1- carboxamido)-3-fluorobenzyl)-5- (4-(dimethylcarbamoyl)phenyl)-1- isopropyl-7-methyl- 4,5,6,7-tetrahydro-1H- pyrazolo[4,3-c]pyridine-3- carboxamide |
| V80 | 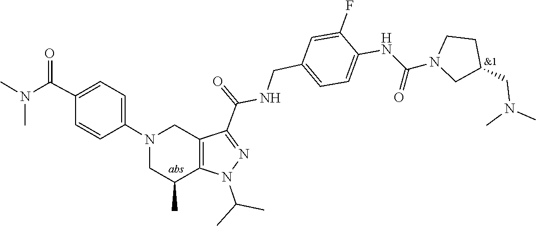 | (S)-N-(4-((RS)-3- ((dimethylamino)methyl)pyrrolidine-1- carboxamido)-3- fluorobenzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V81 | 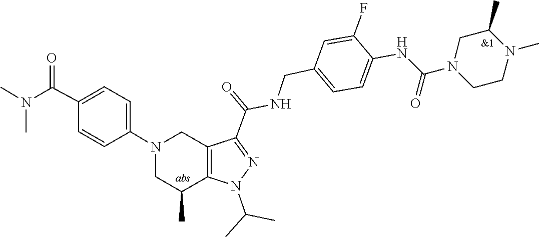 | (S)-5-(4- (dimethylcarbamoyl)phenyl)-N-(4- ((RS)-3,4-dimethylpiperazine-1- carboxamido)-3-fluorobenzyl)-1- isopropyl-7-methyl- 4,5,6,7-tetrahydro-1H- pyrazolo[4,3-c]pyridine-3- carboxamide |
| V82 | 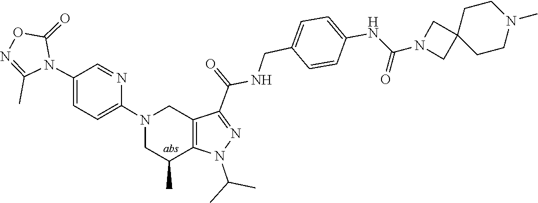 [SMILES: Cc1noc(=O)n1-c1ccc(N2Cc3c(C(=O)NCc4ccc(NC(=O)N5CC6(CCN(C)CC6)C5)cc4)nn(C(C)C)c3[C@@H](C)C2)nc1] | (S)-1-isopropyl-7-methyl-N-(4-(7- methyl-2,7- diazaspiro[3.5]nonane-2- carboxamido)benzyl)-5-(5-(3- methyl-5-oxo-1,2,4-oxadiazol- 4(5H)-yl)pyridin-2-yl)- 4,5,6,7-tetrahydro-1H- pyrazolo[4,3-c]pyridine-3- carboxamide |
| V83 | 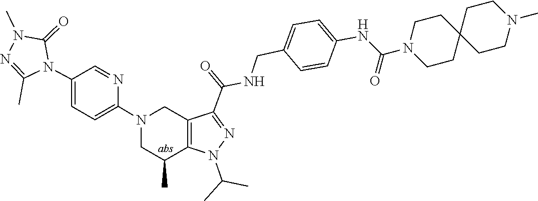 [SMILES: Cc1nn(C)c(=O)n1-c1ccc(N2Cc3c(C(=O)NCc4ccc(NC(=O)N5CCC6(CCN(C)CC6)CC5)cc4)nn(C(C)C)c3[C@@H](C)C2)nc1] | (S)-N-(4-((5-(5-(1,3-dimethyl-5- oxo-1,5-dihydro-4H-1,2,4-triazol- 4-yl)pyridin-2-yl)-1-isopropyl-7- methyl-4,5,6,7-tetrahydro-1H- pyrazolo[4,3-c]pyridine-3- carboxamido)methyl)phenyl)-9- methyl-3,9- diazaspiro[5.5]undecane-3- carboxamide |
| V84 | 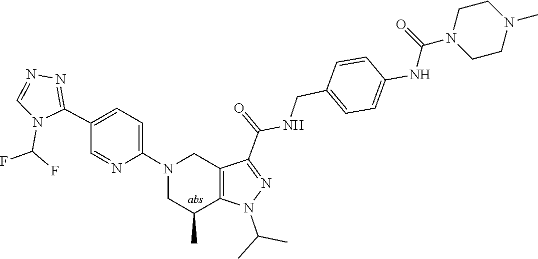 [SMILES: CC(C)n1nc(C(=O)NCc2ccc(NC(=O)N3CCN(C)CC3)cc2)c2c1[C@@H](C)CN(c1ccc(-c3nncn3C(F)F)cn1)C2] | (S)-5-(5-(4-(difluoromethyl)-4H- 1,2,4-triazol-3-yl)pyridin-2-yl)-1- isopropyl-7-methyl-N-(4-(4- methylpiperazine-1- carboxamido)benzyl)- 4,5,6,7-tetrahydro-1H- pyrazolo[4,3-c]pyridine-3- carboxamide |
| V85 | 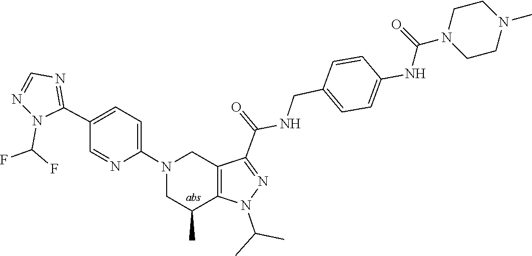 [SMILES: CC(C)n1nc(C(=O)NCc2ccc(NC(=O)N3CCN(C)CC3)cc2)c2c1[C@@H](C)CN(c1ccc(-c3ncnn3C(F)F)cn1)C2] | (S)-5-(5-(1-(difluoromethyl)-1H- 1,2,4-triazol-5-yl)pyridin-2-yl)-1- isopropyl-7-methyl-N-(4-(4- methylpiperazine-1- carboxamido)benzyl)- 4,5,6,7-tetrahydro-1H- pyrazolo[4,3-c]pyridine-3- carboxamide |
| V86 | 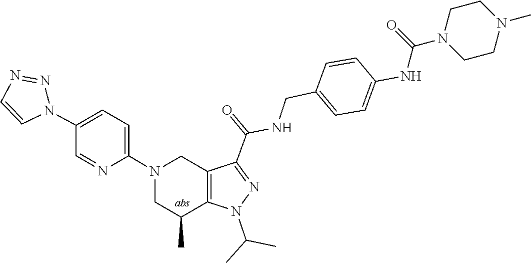 [SMILES: CC(C)n1nc(C(=O)NCc2ccc(NC(=O)N3CCN(C)CC3)cc2)c2c1[C@@H](C)CN(c1ccc(-n3ccnn3)cn1)C2] | (S)-5-(5-(1H-1,2,3-triazol-1- yl)pyridin-2-yl)-1-isopropyl-7- methyl-N-(4-(4-methylpiperazine- 1-carboxamido)benzyl)- 4,5,6,7-tetrahydro-1H- pyrazolo[4,3-c]pyridine-3- carboxamide |
| V87 | 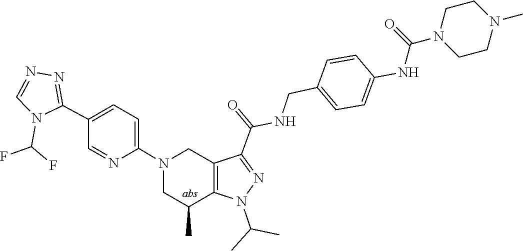 [SMILES: CC(C)n1nc(C(=O)NCc2ccc(NC(=O)N3CCN(C)CC3)cc2)c2c1[C@@H](C)CN(c1ccc(-c3nncn3C(F)F)cn1)C2] | (S)-5-(5-(5-(difluoromethyl)-1H- 1,2,3-triazol-1-yl)pyridin-2-yl)-1- isopropyl-7-methyl-N-(4-(4- methylpiperazine-1- carboxamido)benzyl)- 4,5,6,7-tetrahydro-1H- pyrazolo[4,3-c]pyridine-3- carboxamide |
| V88 | 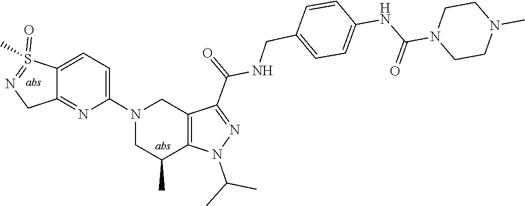 | (S)-1-isopropyl-7-methyl-5-((S)-1- methyl-1-oxido-3H-1l4- benzo[d]isothiazol-5-yl)-N-(4-(4- methylpiperazine-1- carboxamido)benzyl)- 4,5,6,7-tetrahydro-1H- pyrazolo[4,3-c]pyridine-3- carboxamide |
| V89 | 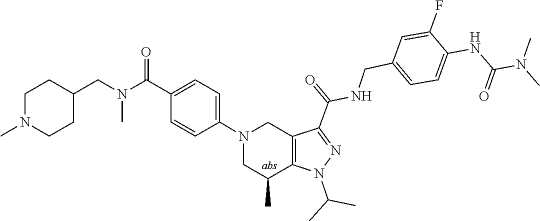 | (S)-N-(4-(3,3-dimethylureido)-3- fluorobenzyl)-1-isopropyl-7- methyl-5-(4-(methyl((1- methylpiperidin-4- yl)methyl)carbamoyl)phenyl)- 4,5,6,7-tetrahydro-1H- pyrazolo[4,3-c]pyridine-3- carboxamide |
| V90 | 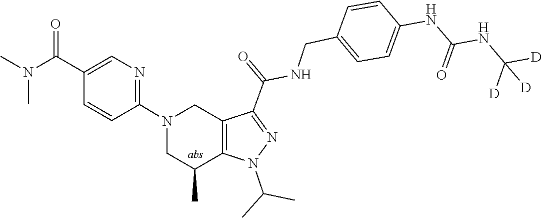 [SMILES: [2H]C([2H])([2H])NC(=O)Nc1ccc(CNC(=O)c2nn(C(C)C)c3c2CN(c2ccc(C(=O)N(C)C)cn2)C[C@@H]3C)cc1] | (S)-5-(5- (dimethylcarbamoyl)pyridin-2-yl)- 1-isopropyl-7-methyl-N-(4-(3- (methyl-d₃)ureido)benzyl)- 4,5,6,7-tetrahydro-1H- pyrazolo[4,3-c]pyridine-3- carboxamide |
| V91 | 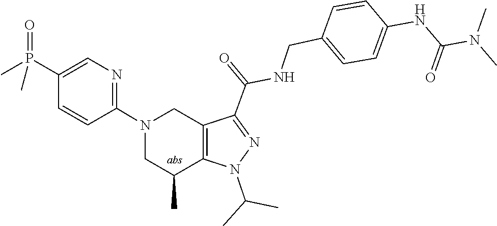 | (S)-5-(5- (dimethylphosphoryl)pyridin-2-yl)- N-(4-(3,3-dimethylureido)benzyl)- 1-isopropyl-7-methyl- 4,5,6,7-tetrahydro-1H- pyrazolo[4,3-c]pyridine-3- carboxamide |
| V92 | 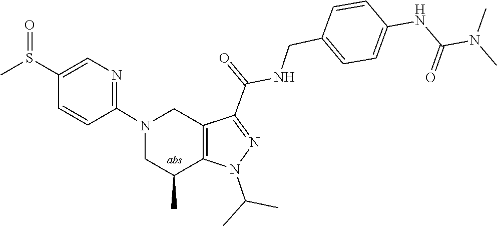 | (7S)-N-(4-(3,3- dimethylureido)benzyl)-1- isopropyl-7-methyl-5-(5- (methylsulfinyl)pyridin-2-yl)- 4,5,6,7-tetrahydro-1H- pyrazolo[4,3-c]pyridine-3- carboxamide |
| V93 | 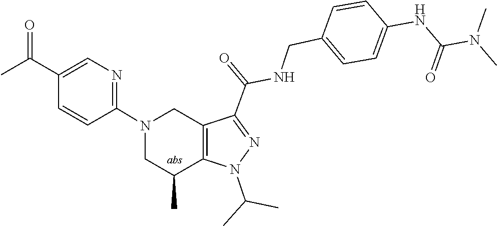 [SMILES: CC(=O)c1ccc(N2Cc3c(C(=O)NCc4ccc(NC(=O)N(C)C)cc4)nn(C(C)C)c3[C@@H](C)C2)nc1] | (S)-5-(5-acetylpyridin-2-yl)-N-(4- (3,3-dimethylureido)benzyl)-1- isopropyl-7-methyl- 4,5,6,7-tetrahydro-1H- pyrazolo[4,3-c]pyridine-3- carboxamide |
| V94 | 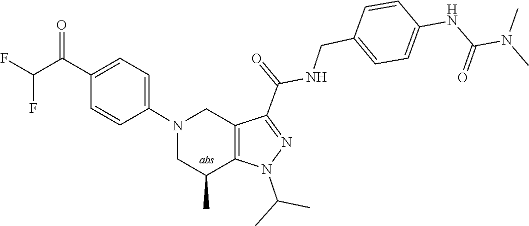 [SMILES: CC(C)n1nc(C(=O)NCc2ccc(NC(=O)N(C)C)cc2)c2c1[C@@H](C)CN(c1ccc(C(=O)C(F)F)cc1)C2] | (S)-5-(4-(2,2- difluoroacetyl)phenyl)-N-(4-(3,3- dimethylureido)benzyl)-1- isopropyl-7-methyl- 4,5,6,7-tetrahydro-1H- pyrazolo[4,3-c]pyridine-3- carboxamide |
| V95 | 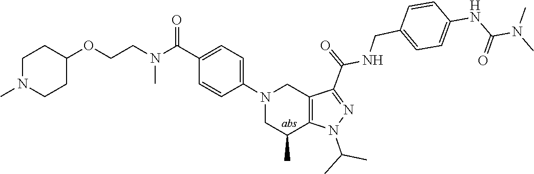 | (S)-N-(4-(3,3- dimethylureido)benzyl)-1- isopropyl-7-methyl-5-(4- (methyl(2-((1-methylpiperidin-4- yl)oxy)ethyl)carbamoyl)phenyl)- 4,5,6,7-tetrahydro-1H- pyrazolo[4,3-c]pyridine-3- carboxamide |
| V96 | 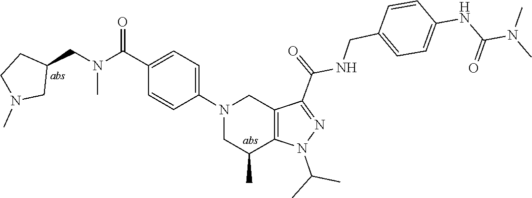 [SMILES: CC(C)n1nc(C(=O)NCc2ccc(NC(=O)N(C)C)cc2)c2c1[C@@H](C)CN(c1ccc(C(=O)N(C)C[C@@H]3CCN(C)C3)cc1)C2] | (S)-N-(4-(3,3- dimethylureido)benzyl)-1- isopropyl-7-methyl-5-(4- (methyl(((R)-1-methylpyrrolidin-3- yl)methyl)carbamoyl)phenyl)- 4,5,6,7-tetrahydro-1H- pyrazolo[4,3-c]pyridine-3- carboxamide |
| V97 | 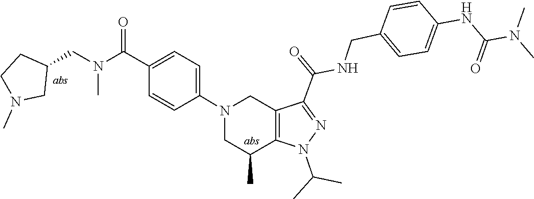 [SMILES: CC(C)n1nc(C(=O)NCc2ccc(NC(=O)N(C)C)cc2)c2c1[C@@H](C)CN(c1ccc(C(=O)N(C)C[C@H]3CCN(C)C3)cc1)C2] | (S)-N-(4-(3,3- dimethylureido)benzyl)-1- isopropyl-7-methyl-5-(4- (methyl(((S)-1-methylpyrrolidin-3- yl)methyl)carbamoyl)phenyl)- 4,5,6,7-tetrahydro-1H- pyrazolo[4,3-c]pyridine-3- carboxamide |
| V98 | 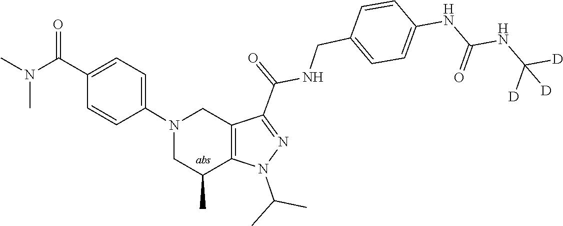 [SMILES: [2H]C([2H])([2H])NC(=O)Nc1ccc(CNC(=O)c2nn(C(C)C)c3c2CN(c2ccc(C(=O)N(C)C)cc2)C[C@@H]3C)cc1] | (S)-5-(4- (dimethylcarbamoyl)phenyl)-1- isopropyl-7-methyl-N-(4-(3- (methyl-d₃)ureido)benzyl)- 4,5,6,7-tetrahydro-1H- pyrazolo[4,3-c]pyridine-3- carboxamide |
| V99 | 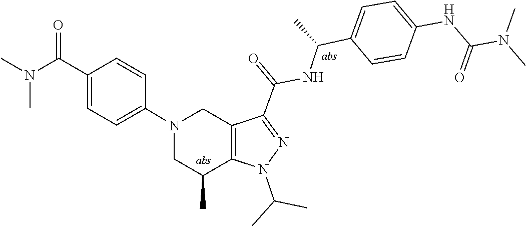 | (S)-5-(5- (dimethylcarbamoyl)pyridin-2-yl)- N-((R)-1-(4-(3,3- dimethylureido)phenyl)ethyl)-1- isopropyl-7-methyl- 4,5,6,7-tetrahydro-1H- pyrazolo[4,3-c]pyridine-3- carboxamide |
| V100 | 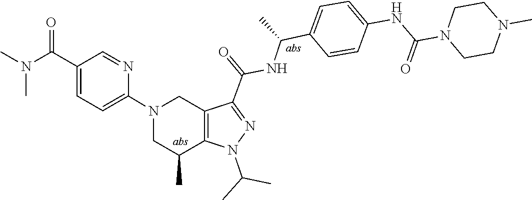 | (S)-5-(5- (dimethylcarbamoyl)pyridin-2-yl)- 1-isopropyl-7-methyl-N-((R)-1-(4- (4-methylpiperazine-1- carboxamido)phenyl)ethyl)- 4,5,6,7-tetrahydro-1H- pyrazolo[4,3-c]pyridine-3- carboxamide |
| V101 | 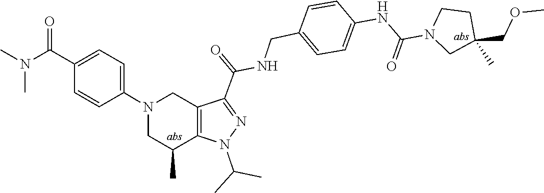 | (S)-N-(4-((S)-3-amino-3- (methoxymethyl)pyrrolidine-1- carboxamido)benzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V102 | 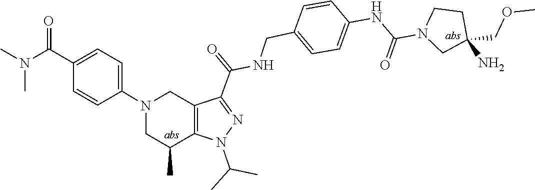 | (S)-N-(4-((R)-3-amino-3- (methoxymethyl)pyrrolidine-1- carboxamido)benzyl)-5-(4- (dimethylcarbamoyl)phenyl)-1- isopropyl-7-methyl- 4,5,6,7-tetrahydro-1H- pyrazolo[4,3-c]pyridine-3- carboxamide |
| V103 | 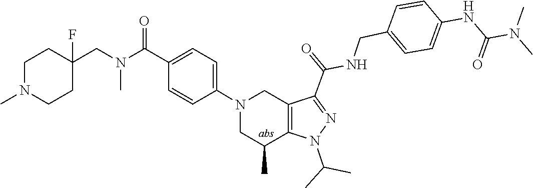 | (S)-N-(4-(3,3- dimethylureido)benzyl)-5-(4-(((4- fluoro-1-methylpiperidin-4- yl)methyl)(methyl)carbamoyl) phenyl)-1-isopropyl-7-methyl- 4,5,6,7-tetrahydro-1H- pyrazolo[4,3-c]pyridine-3- carboxamide |
| V104 | 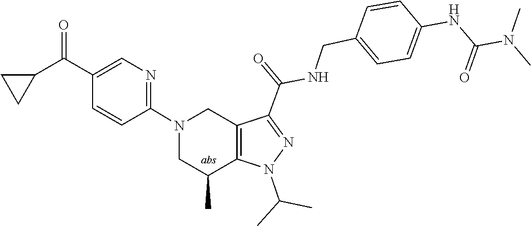 [SMILES: CC(C)n1nc(C(=O)NCc2ccc(NC(=O)N(C)C)cc2)c2c1[C@@H](C)CN(c1ccc(C(=O)C3CC3)cn1)C2] | (S)-5-(5- (cyclopropanecarbonyl)pyridin-2- yl)-N-(4-(3,3- dimethylureido)benzyl)-1- isopropyl-7-methyl- 4,5,6,7-tetrahydro-1H- pyrazolo[4,3-c]pyridine-3- carboxamide |
| V105 | 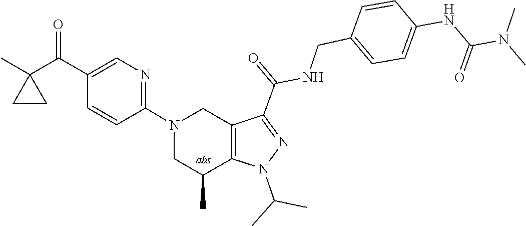 | (S)-N-(4-(3,3- dimethylureido)benzyl)-1- isopropyl-7-methyl-5-(5-(1- methylcyclopropane-1- carbonyl)pyridin-2-yl)- 4,5,6,7-tetrahydro-1H- pyrazolo[4,3-c]pyridine-3- carboxamide |
| V106 | 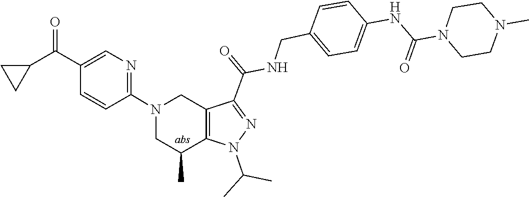 [SMILES: CC(C)n1nc(C(=O)NCc2ccc(NC(=O)N3CCN(C)CC3)cc2)c2c1[C@@H](C)CN(c1ccc(C(=O)C3CC3)cn1)C2] | (S)-5-(5- (cyclopropanecarbonyl)pyridin-2- yl)-1-isopropyl-7-methyl-N-(4-(4- methylpiperazine-1- carboxamido)benzyl)- 4,5,6,7-tetrahydro-1H- pyrazolo[4,3-c]pyridine-3- carboxamide |
| V107 | 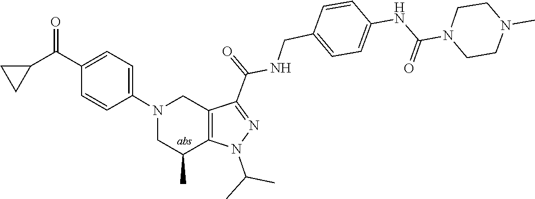 [SMILES: CC(C)n1nc(C(=O)NCc2ccc(NC(=O)N3CCN(C)CC3)cc2)c2c1[C@@H](C)CN(c1ccc(C(=O)C3CC3)cc1)C2] | (S)-5-(4- (cyclopropanecarbonyl)phenyl)-1- isopropyl-7-methyl-N-(4-(4- methylpiperazine-1- carboxamido)benzyl)- 4,5,6,7-tetrahydro-1H- pyrazolo[4,3-c]pyridine-3- carboxamide |
| V108 | 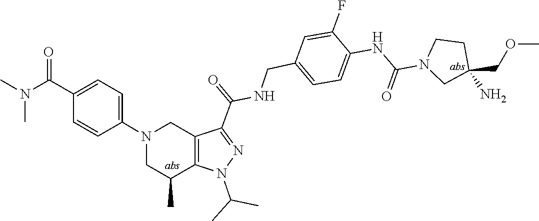 | (S)-N-(4-((S)-3-amino-3- (methoxymethyl)pyrrolidine-1- carboxamido)-3-fluorobenzyl)-5- (4-(dimethylcarbamoyl)phenyl)-1- isopropyl-7-methyl- 4,5,6,7-tetrahydro-1H- pyrazolo[4,3-c]pyridine-3- carboxamide |
| V109 | 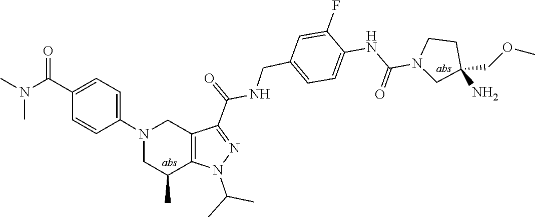 | (S)-N-(4-((R)-3-amino-3- (methoxymethyl)pyrrolidine-1- carboxamido)-3-fluorobenzyl)-5- (4-(dimethylcarbamoyl)phenyl)-1- isopropyl-7-methyl- 4,5,6,7-tetrahydro-1H- pyrazolo[4,3-c]pyridine-3- carboxamide |
| V110 | 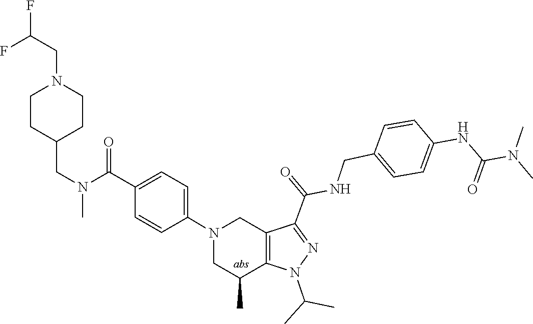 [SMILES: CC(C)n1nc(C(=O)NCc2ccc(NC(=O)N(C)C)cc2)c2c1[C@@H](C)CN(c1ccc(C(=O)N(C)CC3CCN(CC(F)F)CC3)cc1)C2] | (S)-5-(4-(((1-(2,2- difluoroethyl)piperidin-4- yl)methyl)(methyl)carbamoyl) phenyl)-N-(4-(3,3- dimethylureido)benzyl)-1- isopropyl-7-methyl- 4,5,6,7-tetrahydro-1H- pyrazolo[4,3-c]pyridine-3- carboxamide |
| V111 | 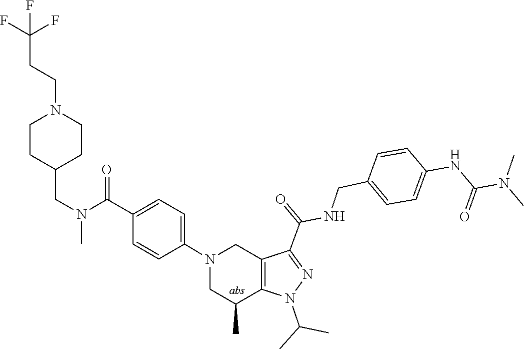 [SMILES: CC(C)n1nc(C(=O)NCc2ccc(NC(=O)N(C)C)cc2)c2c1[C@@H](C)CN(c1ccc(C(=O)N(C)CC3CCN(CCC(F)(F)F)CC3)cc1)C2] | (S)-N-(4-(3,3- dimethylureido)benzyl)-1- isopropyl-7-methyl-5-(4- (methyl((1-(3,3,3- trifluoropropyl)piperidin-4- yl)methyl)carbamoyl)phenyl)- 4,5,6,7-tetrahydro-1H- pyrazolo[4,3-c]pyridine-3- carboxamide |
| V112 | 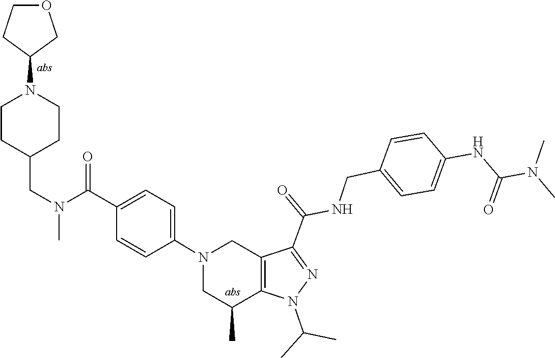 [SMILES: CC(C)n1nc(C(=O)NCc2ccc(NC(=O)N(C)C)cc2)c2c1[C@@H](C)CN(c1ccc(C(=O)N(C)CC3CCN([C@H]4CCOC4)CC3)cc1)C2] | (S)-N-(4-(3,3- dimethylureido)benzyl)-1- isopropyl-7-methyl-5-(4- (methyl((1-((S)-tetrahydrofuran-3- yl)piperidin-4- yl)methyl)carbamoyl)phenyl)- 4,5,6,7-tetrahydro-1H- pyrazolo[4,3-c]pyridine-3- carboxamide |
| V113 | 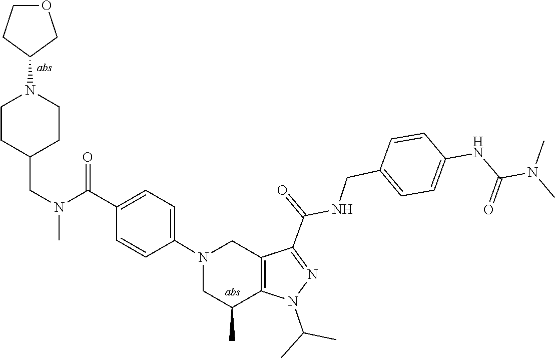 [SMILES: CC(C)n1nc(C(=O)NCc2ccc(NC(=O)N(C)C)cc2)c2c1[C@@H](C)CN(c1ccc(C(=O)N(C)CC3CCN([C@@H]4CCOC4)CC3)cc1)C2] | (S)-N-(4-(3,3- dimethylureido)benzyl)-1- isopropyl-7-methyl-5-(4- (methyl((1-((R)-tetrahydrofuran-3- yl)piperidin-4- yl)methyl)carbamoyl)phenyl)- 4,5,6,7-tetrahydro-1H- pyrazolo[4,3-c]pyridine-3- carboxamide |
| V114 | 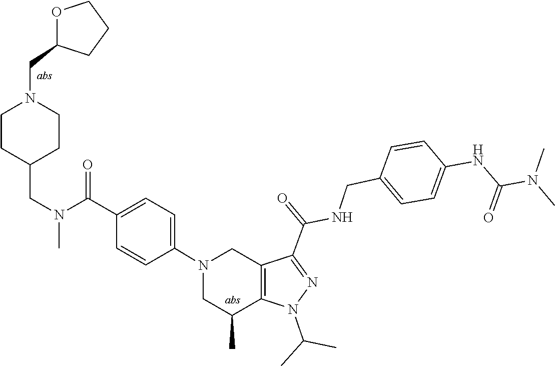 [SMILES: CC(C)n1nc(C(=O)NCc2ccc(NC(=O)N(C)C)cc2)c2c1[C@@H](C)CN(c1ccc(C(=O)N(C)CC3CCN(C[C@@H]4CCCO4)CC3)cc1)C2] | (S)-N-(4-(3,3- dimethylureido)benzyl)-1- isopropyl-7-methyl-5-(4- (methyl((1-(((S)-tetrahydrofuran- 2-yl)methyl)piperidin-4- yl)methyl)carbamoyl)phenyl)- 4,5,6,7-tetrahydro-1H- pyrazolo[4,3-c]pyridine-3- carboxamide |
| V115 | 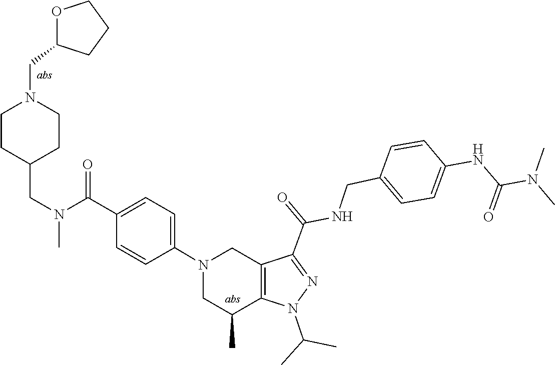 [SMILES: CC(C)n1nc(C(=O)NCc2ccc(NC(=O)N(C)C)cc2)c2c1[C@@H](C)CN(c1ccc(C(=O)N(C)CC3CCN(C[C@H]4CCCO4)CC3)cc1)C2] | (S)-N-(4-(3,3- dimethylureido)benzyl)-1- isopropyl-7-methyl-5-(4- (methyl((1-(((R)-tetrahydrofuran- 2-yl)methyl)piperidin-4- yl)methyl)carbamoyl)phenyl)- 4,5,6,7-tetrahydro-1H- pyrazolo[4,3-c]pyridine-3- carboxamide |
| V116 | 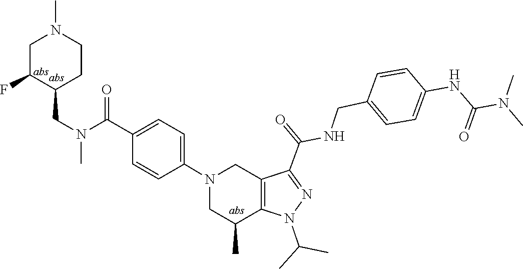 [SMILES: CC(C)n1nc(C(=O)NCc2ccc(NC(=O)N(C)C)cc2)c2c1[C@@H](C)CN(c1ccc(C(=O)N(C)C[C@@H]3CCN(C)C[C@@H]3F)cc1)C2] | (S)-N-(4-(3,3- dimethylureido)benzyl)-5-(4- ((((3R,4S)-3-fluoro-1- methylpiperidin-4- yl)methyl)(methyl)carbamoyl) phenyl)-1-isopropyl-7-methyl- 4,5,6,7-tetrahydro-1H- pyrazolo[4,3-c]pyridine-3- carboxamide |
| V117 | 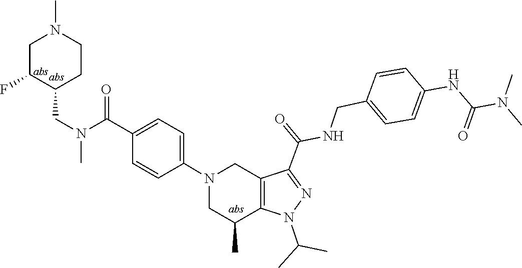 [SMILES: CC(C)n1nc(C(=O)NCc2ccc(NC(=O)N(C)C)cc2)c2c1[C@@H](C)CN(c1ccc(C(=O)N(C)C[C@H]3CCN(C)C[C@H]3F)cc1)C2] | (S)-N-(4-(3,3- dimethylureido)benzyl)-5-(4- ((((3S,4R)-3-fluoro-1- methylpiperidin-4- yl)methyl)(methyl)carbamoyl) phenyl)-1-isopropyl-7-methyl- 4,5,6,7-tetrahydro-1H- pyrazolo[4,3-c]pyridine-3- carboxamide |
| V118 | 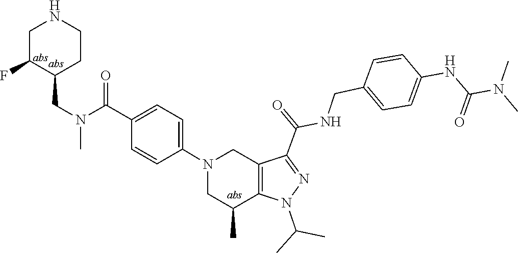 [SMILES: CC(C)n1nc(C(=O)NCc2ccc(NC(=O)N(C)C)cc2)c2c1[C@@H](C)CN(c1ccc(C(=O)N(C)C[C@@H]3CCNC[C@@H]3F)cc1)C2] | (S)-N-(4-(3,3- dimethylureido)benzyl)-5-(4- ((((3R,4S)-3-fluoropiperidin-4- yl)methyl)(methyl)carbamoyl) phenyl)-1-isopropyl-7-methyl- 4,5,6,7-tetrahydro-1H- pyrazolo[4,3-c]pyridine-3- carboxamide |
| V119 | 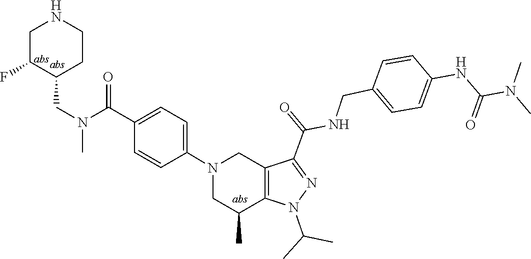 [SMILES: CC(C)n1nc(C(=O)NCc2ccc(NC(=O)N(C)C)cc2)c2c1[C@@H](C)CN(c1ccc(C(=O)N(C)C[C@H]3CCNC[C@H]3F)cc1)C2] | (S)-N-(4-(3,3- dimethylureido)benzyl)-5-(4- ((((3S,4R)-3-fluoropiperidin-4- yl)methyl)(methyl)carbamoyl) phenyl)-1-isopropyl-7-methyl- 4,5,6,7-tetrahydro-1H- pyrazolo[4,3-c]pyridine-3- carboxamide |
| V120 | 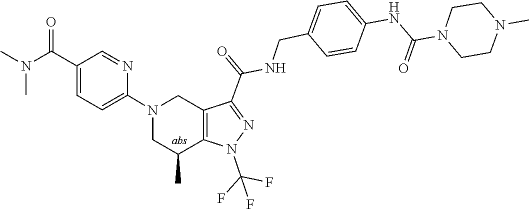 | (S)-5-(5- (dimethylcarbamoyl)pyridin-2-yl)- 7-methyl-N-(4-(4- methylpiperazine-1- carboxamido)benzyl)-1- (trifluoromethyl)- 4,5,6,7-tetrahydro-1H- pyrazolo[4,3-c]pyridine-3- carboxamide |
| V121 | 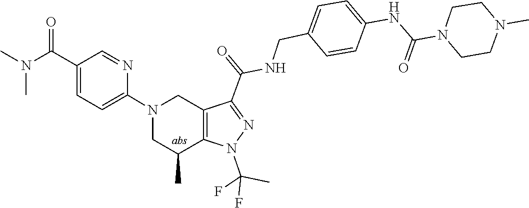 | (S)-1-(1,1-difluoroethyl)-5-(5- (dimethylcarbamoyl)pyridin-2-yl)- 7-methyl-N-(4-(4- methylpiperazine-1- carboxamido)benzyl)- 4,5,6,7-tetrahydro-1H- pyrazolo[4,3-c]pyridine-3- carboxamide |
| V122 | 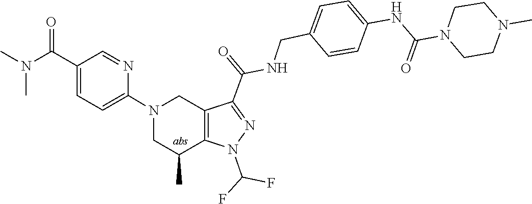 | (S)-1-(difluoromethyl)-5-(5- (dimethylcarbamoyl)pyridin-2-yl)- 7-methyl-N-(4-(4- methylpiperazine-1- carboxamido)benzyl)- 4,5,6,7-tetrahydro-1H- pyrazolo[4,3-c]pyridine-3- carboxamide |
| V123 | 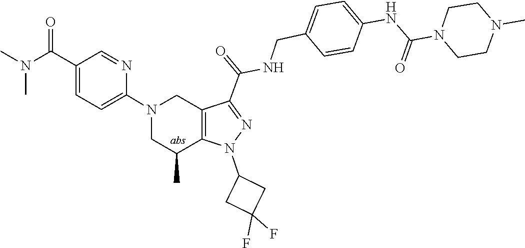 | (S)-1-(3,3-difluorocyclobutyl)-5- (5-(dimethylcarbamoyl)pyridin-2- yl)-7-methyl-N-(4-(4- methylpiperazine-1- carboxamido)benzyl)- 4,5,6,7-tetrahydro-1H- pyrazolo[4,3-c]pyridine-3- carboxamide |
| V124 | 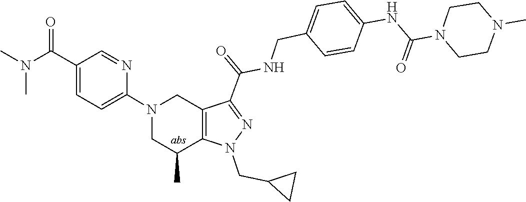 | (S)-1-(cyclopropylmethyl)-5-(5- (dimethylcarbamoyl)pyridin-2-yl)- 7-methyl-N-(4-(4- methylpiperazine-1- carboxamido)benzyl)- 4,5,6,7-tetrahydro-1H- pyrazolo[4,3-c]pyridine-3- carboxamide |
| V125 | 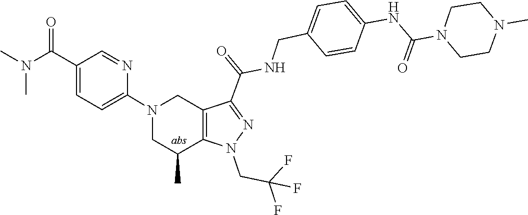 | (S)-5-(5- (dimethylcarbamoyl)pyridin-2-yl)- 7-methyl-N-(4-(4- methylpiperazine-1- carboxamido)benzyl)-1-(2,2,2- trifluoroethyl)- 4,5,6,7-tetrahydro-1H- pyrazolo[4,3-c]pyridine-3- carboxamide |
| V126 | 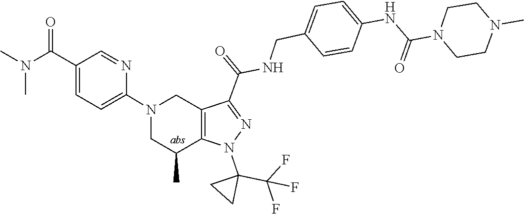 | (S)-5-(5- (dimethylcarbamoyl)pyridin-2-yl)- 7-methyl-N-(4-(4- methylpiperazine-1- carboxamido)benzyl)-1-(1- (trifluoromethyl)cyclopropyl)- 4,5,6,7-tetrahydro-1H- pyrazolo[4,3-c]pyridine-3- carboxamide |
| V127 | 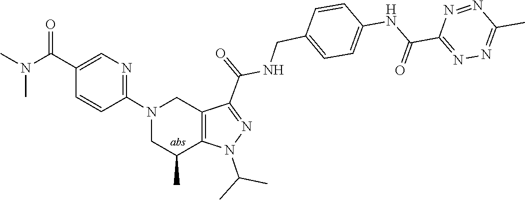 | (S)-5-(5- (dimethylcarbamoyl)pyridin-2-yl)- 1-isopropyl-7-methyl-N-(4-(6- methyl-1,2,4,5-tetrazin-3- yl)benzyl)-4,5,6,7-tetrahydro-1H- pyrazolo[4,3-c]pyridine-3- carboxamide |
| V128 | 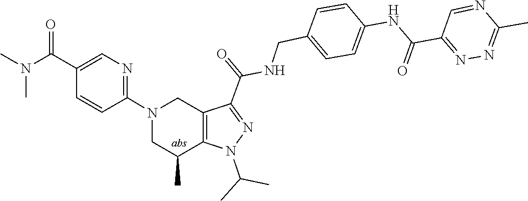 | (S)-5-(5- (dimethylcarbamoyl)pyridin-2-yl)- 1-isopropyl-7-methyl-N-(4-(3- methyl-1,2,4-triazin-6-yl)benzyl)- 4,5,6,7-tetrahydro-1H- pyrazolo[4,3-c]pyridine-3- carboxamide |
| V129 | 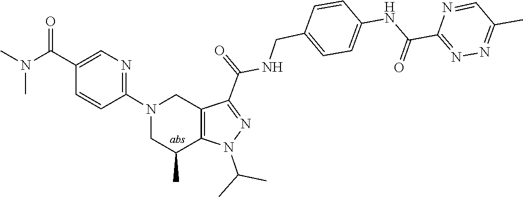 | (S)-5-(5- (dimethylcarbamoyl)pyridin-2-yl)- 1-isopropyl-7-methyl-N-(4-(6- methyl-1,2,4-triazin-3-yl)benzyl)- 4,5,6,7-tetrahydro-1H- pyrazolo[4,3-c]pyridine-3- carboxamide |
| V130 | 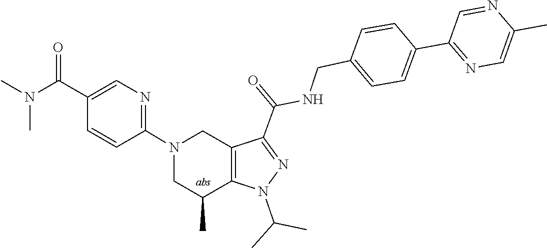 | (S)-5-(5- (dimethylcarbamoyl)pyridin-2-yl)- 1-isopropyl-7-methyl-N-(4-(5- methylpyrazin-2-yl)benzyl)- 4,5,6,7-tetrahydro-1H- pyrazolo[4,3-c]pyridine-3- carboxamide |
| V131 | 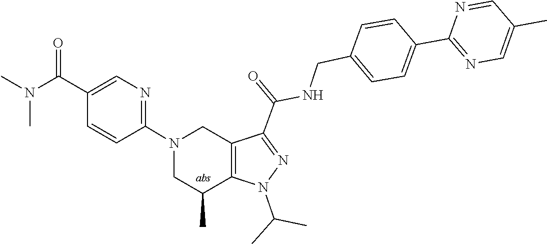 | (S)-5-(5- (dimethylcarbamoyl)pyridin-2-yl)- 1-isopropyl-7-methyl-N-(4-(5- methylpyrimidin-2-yl)benzyl)- 4,5,6,7-tetrahydro-1H- pyrazolo[4,3-c]pyridine-3- carboxamide |
| V132 | 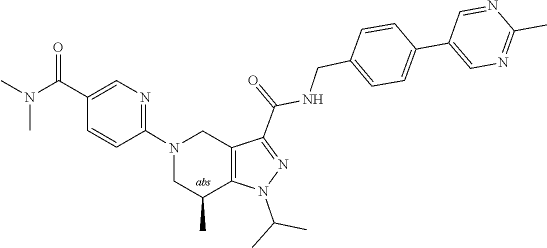 | (S)-5-(5- (dimethylcarbamoyl)pyridin-2-yl)- 1-isopropyl-7-methyl-N-(4-(2- methylpyrimidin-5-yl)benzyl)- 4,5,6,7-tetrahydro-1H- pyrazolo[4,3-c]pyridine-3- carboxamide |
| V133 | 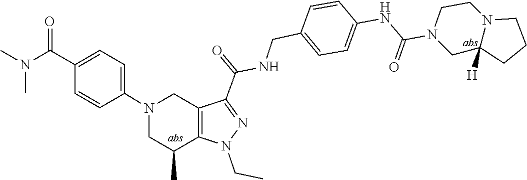 [SMILES: CCn1nc(C(=O)NCc2ccc(NC(=O)N3CCN4CCC[C@@H]4C3)cc2)c2c1[C@@H](C)CN(c1ccc(C(=O)N(C)C)cc1)C2] | (S)-5-(4- (dimethylcarbamoyl)phenyl)-1- ethyl-7-methyl-N-(4-((R)- octahydropyrrolo[1,2-a]pyrazine- 2-carboxamido)benzyl)- 4,5,6,7-tetrahydro-1H- pyrazolo[4,3-c]pyridine-3- carboxamide |
| V134 | 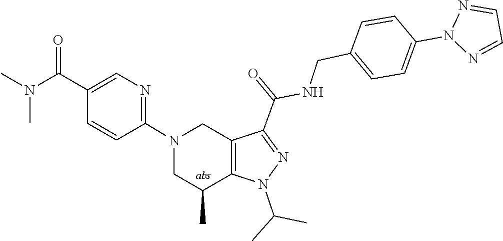 [SMILES: CC(C)n1nc(C(=O)NCc2ccc(-n3nccn3)cc2)c2c1[C@@H](C)CN(c1ccc(C(=O)N(C)C)cn1)C2] | (S)-N-(4-(2H-1,2,3-triazol-2- yl)benzyl)-5-(5- (dimethylcarbamoyl)pyridin-2-yl)- 1-isopropyl-7-methyl- 4,5,6,7-tetrahydro-1H- pyrazolo[4,3-c]pyridine-3- carboxamide |
| V135 | 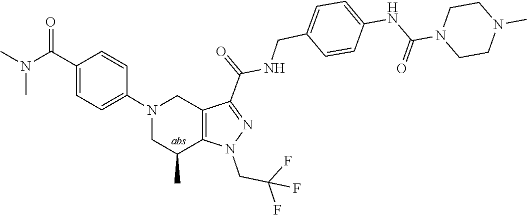 | (S)-5-(4- (dimethylcarbamoyl)phenyl)-7- methyl-N-(4-(4-methylpiperazine- 1-carboxamido)benzyl)-1-(2,2,2- trifluoroethyl)- 4,5,6,7-tetrahydro-1H- pyrazolo[4,3-c]pyridine-3- carboxamide |
| V136 | 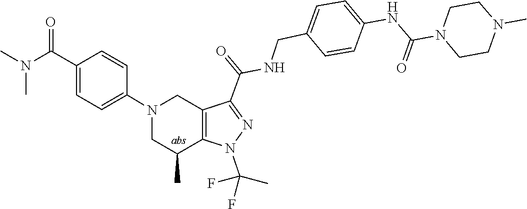 | (S)-1-(1,1-difluoroethyl)-5-(4- (dimethylcarbamoyl)phenyl)-7- methyl-N-(4-(4-methylpiperazine- 1-carboxamido)benzyl)- 4,5,6,7-tetrahydro-1H- pyrazolo[4,3-c]pyridine-3- carboxamide |
| V137 | 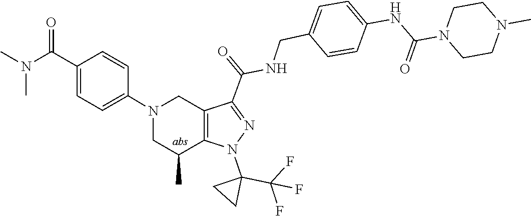 | (S)-5-(4- (dimethylcarbamoyl)phenyl)-7- methyl-N-(4-(4-methylpiperazine- 1-carboxamido)benzyl)-1-(1- (trifluoromethyl)cyclopropyl)- 4,5,6,7-tetrahydro-1H- pyrazolo[4,3-c]pyridine-3- carboxamide |
| V138 | 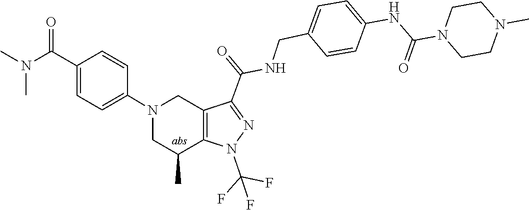 | (S)-5-(4- (dimethylcarbamoyl)phenyl)-7- methyl-N-(4-(4-methylpiperazine- 1-carboxamido)benzyl)-1- (trifluoromethyl)- 4,5,6,7-tetrahydro-1H- pyrazolo[4,3-c]pyridine-3- carboxamide |
| V139 | 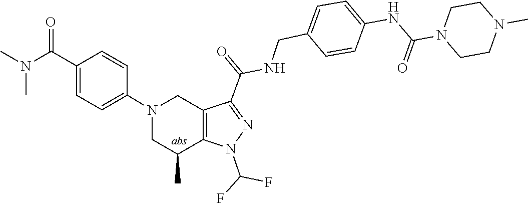 | (S)-1-(difluoromethyl)-5-(4- (dimethylcarbamoyl)phenyl)-7- methyl-N-(4-(4-methylpiperazine- 1-carboxamido)benzyl)- 4,5,6,7-tetrahydro-1H- pyrazolo[4,3-c]pyridine-3- carboxamide |
| V140 | 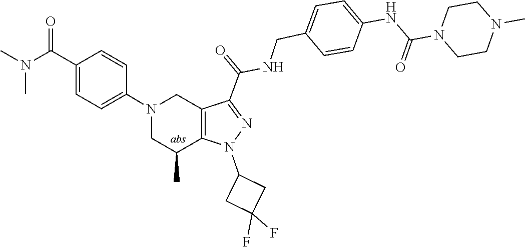 | (S)-1-(3,3-difluorocyclobutyl)-5- (4-(dimethylcarbamoyl)phenyl)-7- methyl-N-(4-(4-methylpiperazine- 1-carboxamido)benzyl)- 4,5,6,7-tetrahydro-1H- pyrazolo[4,3-c]pyridine-3- carboxamide |
| V141 | 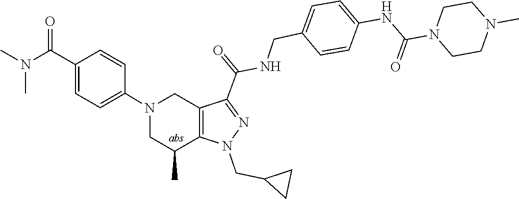 | (S)-1-(cyclopropylmethyl)-5-(4- (dimethylcarbamoyl)phenyl)-7- methyl-N-(4-(4-methylpiperazine- 1-carboxamido)benzyl)- 4,5,6,7-tetrahydro-1H- pyrazolo[4,3-c]pyridine-3- carboxamide |
| V142 | 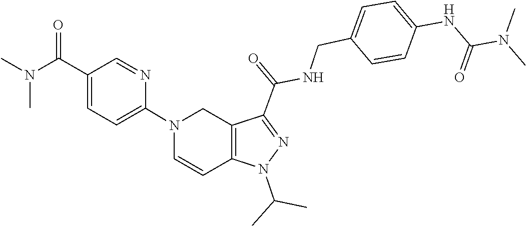 | 5-(5-(dimethylcarbamoyl)pyridin- 2-yl)-N-(4-(3,3- dimethylureido)benzyl)-1- isopropyl-4,5-dihydro-1H- pyrazolo[4,3-c]pyridine-3- carboxamide |

### Pharmacology Assays

### STAT6 Reporter Luciferase Assay

Pharmacological measurements of STAT6 transcriptional activity through the IL-13-IL-13 receptor/IL-4 receptor pathway were made with a stably transfected HaCaT keratinocyte cell line, which expresses NanoLuc luciferase reporter gene under the transcriptional control of the STAT6 responsive promoter for IL-13Ra2. The readout of the STAT6 activity utilized Promega Nano-Glo Luciferase assay kit.

The reporter cells were cultured in a growth media comprised of DMEM High Glucose, no calcium and no glutamine, 10% heat inactivated fetal bovine serum (HI FBS), 1% 100× Glutamax, and 100 U/mL Penicillin-Streptomycin (units of penicillin in 1 mL of streptomycin). Seven thousand cells were plated in 25 microliter (μL) per well in a 384-well, white bottom, tissue culture plate. Cells were cultured overnight at 37° C. The next day, test compounds (solubilized and serially diluted in DMSO, in an 11 point, 4-fold dilution series in duplicate) were spotted, 60 nanoliters (nL) per well, into a 384 well v-bottom polypropylene plate. Thirty μL per well of 37° C. dosing media (Dulbecco's modified eagle medium (DMEM) High Glucose, no calcium and no glutamine with 2% HI FBS) was added to the compound plate. Growth media was removed from the cell plate, and the cells were washed with DPBS. Twenty μL per well of compounds diluted in dosing media were transferred from the compound plate to the cell plate and the compounds were further diluted with an additional 15 μL of dosing media in the cell plate: the cell plate was incubated for two hours at 37° C. After two hours, STAT6 activity was stimulated with the addition of 5 μL per well of IL-13 (R&D Systems) in dosing media with a final concentration of 0.12 nM IL13. Cells were incubated at 37° C. for 4 hours to allow for reporter gene expression, then all media was removed from the plate. The cell plates were treated with assay buffer and Luciferase substrate from Nano-Glo Luciferase Assay Kit for 15 min and then measured for luminescence on an EnVision 2105 multilabel reader (PerkinElmer). EC₅₀ (half maximal effective concentration) values were determined from this data using a 4-parameter fit algorithm and are provided in Table 2, where the EC₅₀ values are the statistical mean values of n number of samples as denoted by n(EC₅₀).

### Human Whole Blood (HWB) CCL17 TR-FRET Assay

The IC₅₀ (half-maximal concentration required for inhibition) of compounds for the inhibition of CCL17 secretion in peripheral human whole blood, stimulated through the IL-13-IL-13 receptor/IL-4 receptor pathway was evaluated with a TR-FRET sandwich ELISA CCL17 (TARC) Assay Kit (Bioauxilium).

Test compounds (solubilized and serially diluted in DMSO, in an 11 point, 3-fold dilution series in duplicate) were spotted, 80 nL per well, into a 384 well v-bottom polypropylene plate. Human whole blood was collected from a peripheral vein, 714 units of sodium heparin was added for every 50 mL of blood collected. Sixty μL per well of blood was added to the compound plate. The blood plate was incubated for two hours at 37° C. After two hours, CCL17 production was stimulated with the addition of 20 μL per well of IL13 (R&D Systems) in HBSS with calcium and magnesium, with a final concentration of 10 ng/mL IL13. The plate containing blood, compound, and IL-13 was incubated at 37° C. for 48 hours, to allow for CCL17 production. After 48 hours, the plate was centrifuged at 1,500 revolutions per minute (RPM) for 7 minutes, with no braking. The CCL17 TR-FRET Assay Kit detection reagent was prepared according to kit instructions and 4 μL per well of the antibody cocktail was added to a 384-well white low volume non-binding coated plate (Corning). Sixteen μL of serum from the centrifuged blood plate was transferred to the detection plate containing the TR-FRET antibody cocktail. The plate was centrifuged briefly, then covered and incubated 16 hours at room temperature. The plate was then read on an EnVision 2105 multilabel reader (PerkinElmer) with settings for TR-FRET Ratio=10,000×(fluorescence intensity 665 nm/fluorescence intensity 615 nm). IC₅₀ values were determined from this data using a 4-parameter fit algorithm and are shown in Table 3, where the EC₅₀ values are the statistical mean values of n number of samples as denoted by n(IC₅₀).

TABLE 3

| | | | HWB | | |
|---|---|---|---|---|---|
| | Reporter | | CCL17 | | |
| Ex. | EC₅₀ (μM) | n (EC₅₀) | IC₅₀ (μM) | n (IC₅₀) | ChemDraw Name |
| 1 | 0.011 | 2 | 0.318 | 6 | N-(4-(3,3-dimethylureido)benzyl)-1- |
| | | | | | isopropyl-5-(5-(2-oxopyrrolidin-1-yl)pyridin- |
| | | | | | 2-yl)-4,5,6,7-tetrahydro-1H-pyrazolo[4,3- |
| | | | | | c]pyridine-3-carboxamide |
| 2 | 0.008 | 72 | 0.069 | 172 | 5-(5-(Dimethylcarbamoyl)pyridin-2-yl)-N-(4- |
| | | | | | (3,3-dimethylureido)benzyl)-1-isopropyl- |
| | | | | | 4,5,6,7-tetrahydro-1H-pyrazolo[4,3- |
| | | | | | c]pyridine-3-carboxamide |
| 3 | 0.010 | 6 | 0.104 | 10 | 5-(5-(Dimethylcarbamoyl)pyridin-2-yl)-N-(4- |
| | | | | | (3,3-dimethylureido)benzyl)-1-ethyl-4,5,6,7- |
| | | | | | tetrahydro-1H-pyrazolo[4,3-c]pyridine-3- |
| | | | | | carboxamide |
| 4 | 0.178 | 2 | 1.108 | 2 | 5-(4-(Dimethylcarbamoyl)phenyl)-1-methyl- |
| | | | | | N-(4-(methylcarbamoyl)benzyl)-4,5,6,7- |
| | | | | | tetrahydro-1H-pyrazolo[4,3-c]pyridine-3- |
| | | | | | carboxamide |
| 5 | 0.124 | 2 | 0.927 | 7 | 7-(5-(Dimethylcarbamoyl)pyridin-2-yl)-N-(4- |
| | | | | | (3,3-dimethylureido)benzyl)-3-methyl- |
| | | | | | 5,6,7,8-tetrahydroimidazo[1,5-a]pyrazine-1- |
| | | | | | carboxamide |
| 6 | 0.004 | 32 | 0.025 | 72 | rac-(R)-5-(5-(Dimethylcarbamoyl)pyridin-2- |
| | | | | | yl)-N-(4-(3,3-dimethylureido)benzyl)-1,7- |
| | | | | | dimethyl-4,5,6,7-tetrahydro-1H- |
| | | | | | pyrazolo[4,3-c]pyridine-3-carboxamide |
| 7 | 0.033 | 5 | 0.196 | 6 | rel-(R or S)-5-(5- |
| | | | | | (Dimethylcarbamoyl)pyridin-2-yl)-N-(4-(3,3- |
| | | | | | dimethylureido)benzyl)-1,7-dimethyl- |
| | | | | | 4,5,6,7-tetrahydro-1H-pyrazolo[4,3- |
| | | | | | c]pyridine-3-carboxamide |
| 8 | 0.003 | 4 | 0.014 | 6 | rel-(R or S)-5-(5- |
| | | | | | (Dimethylcarbamoyl)pyridin-2-yl)-N-(4-(3,3- |
| | | | | | dimethylureido)benzyl)-1,7-dimethyl- |
| | | | | | 4,5,6,7-tetrahydro-1H-pyrazolo[4,3- |
| | | | | | c]pyridine-3-carboxamide |
| 9 | 0.015 | 2 | 0.027 | 3 | rac-(R)-7-(5-(Dimethylcarbamoyl)pyridin-2- |
| | | | | | yl)-N-(4-(3,3-dimethylureido)benzyl)-3,5- |
| | | | | | dimethyl-5,6,7,8-tetrahydroimidazo[1,5- |
| | | | | | a]lpyrazine-1-carboxamide formate |
| 10 | 0.011 | 2 | 0.094 | 6 | 1-cyclopropyl-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 11 | 0.016 | 3 | 0.205 | 2 | N-(4-(1H-imidazol-2-yl)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 12 | 0.016 | 2 | 0.093 | 3 | N-(4-(3,3-dimethylureido)benzyl)-1-methyl- |
| | | | | | 5-(5-(1-methyl-1H-imidazol-2-yl)pyridin-2- |
| | | | | | yl)-4,5,6,7-tetrahydro-1H-pyrazolo[4,3- |
| | | | | | c]pyridine-3-carboxamide |
| 13 | 0.017 | 2 | 0.083 | 7 | 3-cyclopropyl-7-(5- |
| | | | | | (dimethylcarbamoyl)pyridin-2-yl)-N-(4-(3,3- |
| | | | | | dimethylureido)benzyl)-5,6,7,8- |
| | | | | | tetrahydroimidazo[1,5-a]pyrazine-1- |
| | | | | | carboxamide |
| 14 | 0.029 | 4 | 0.260 | 7 | 5-(5-(dimethylcarbamoyl)pyridin-2-yl)-N-(4- |
| | | | | | (3,3-dimethylureido)benzyl)-1-methyl- |
| | | | | | 4,5,6,7-tetrahydro-1H-pyrazolo[4,3- |
| | | | | | c]pyridine-3-carboxamide |
| 15 | 0.159 | 2 | 1.162 | 6 | 5-(5-(dimethylcarbamoyl)pyridin-2-yl)-1- |
| | | | | | methyl-N-(4-(methylcarbamoyl)benzyl)- |
| | | | | | 4,5,6,7-tetrahydro-1H-pyrazolo[4,3- |
| | | | | | c]pyridine-3-carboxamide |
| 16 | 0.261 | 2 | 0.791 | 2 | 3-cyclopropyl-7-(5- |
| | | | | | (dimethylcarbamoyl)pyridin-2-yl)-N-(4- |
| | | | | | (methylcarbamoyl)benzyl)-5,6,7,8- |
| | | | | | tetrahydroimidazo[1,5-a]pyrazine-1- |
| | | | | | carboxamide |
| 17 | 0.322 | 2 | 0.291 | 3 | 7-(5-(dimethylcarbamoyl)pyridin-2-yl)-N-(4- |
| | | | | | (3,3-dimethylureido)benzyl)-5,6,7,8- |
| | | | | | tetrahydroimidazo[1,5-a]pyrazine-1- |
| | | | | | carboxamide |
| 18 | 1.421 | 2 | 1.379 | 5 | 5-(5-(dimethylcarbamoyl)pyridin-2-yl)-N-(4- |
| | | | | | (3,3-dimethylureido)benzyl)-4,5,6,7- |
| | | | | | tetrahydro-1H-pyrazolo[4,3-c]pyridine-3- |
| | | | | | carboxamide |
| 19 | >10.000 | 1 | 3.364 | 2 | 2-cyclopropyl-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 2H-pyrazolo[4,3-c]pyridine-3-carboxamide |
| 20 | 0.006 | 2 | 0.072 | 3 | 5-(5-(dimethylcarbamoyl)pyridin-2-yl)-N-(4- |
| | | | | | (3,3-dimethylureido)benzyl)-1-(1- |
| | | | | | methylcyclopropyl)-4,5,6,7-tetrahydro-1H- |
| | | | | | pyrazolo[4,3-c]pyridine-3-carboxamide |
| 21 | 0.033 | 4 | 0.217 | 4 | N-(4-(1H-imidazol-2-yl)benzyl)-5-(5- |
| | | | | | (dimethylcarbamoyl)pyridin-2-yl)-1-ethyl- |
| | | | | | 4,5,6,7-tetrahydro-1H-pyrazolo[4,3- |
| | | | | | c]pyridine-3-carboxamide |
| 22 | 0.021 | 2 | 0.054 | 2 | 7-(5-(dimethylcarbamoyl)pyridin-2-yl)-N-(4- |
| | | | | | (3,3-dimethylureido)benzyl)-3- |
| | | | | | (trifluoromethyl)-5,6,7,8- |
| | | | | | tetrahydroimidazo[1,5-a]pyrazine-1- |
| | | | | | carboxamide |
| 23 | 0.006 | 2 | 0.062 | 2 | N-(4-(3,3-dimethylureido)benzyl)-1-ethyl-5- |
| | | | | | (5-(1-methyl-1H-1,2,4-triazol-5-yl)pyridin-2- |
| | | | | | yl)-4,5,6,7-tetrahydro-1H-pyrazolo[4,3- |
| | | | | | c]pyridine-3-carboxamide |
| 24 | 0.384 | 2 | 0.346 | 2 | 5-(5-(dimethylcarbamoyl)pyridin-2-yl)-N-(4- |
| | | | | | (3,3-dimethylureido)benzyl)-4,5,6,7- |
| | | | | | tetrahydro-[1,2,3]triazolo[1,5-a]pyrazine-3- |
| | | | | | carboxamide |
| 25 | 0.164 | 2 | 1.487 | 2 | 5-(5-(dimethylcarbamoyl)pyridin-2-yl)-N-(4- |
| | | | | | (3,3-dimethylureido)benzyl)-4,5,6,7- |
| | | | | | tetrahydrothieno[3,2-c]pyridine-3- |
| | | | | | carboxamide |
| 26 | 0.069 | 2 | 0.561 | 2 | rac-(R)-5-(4-(dimethylcarbamoyl)phenyl)-N- |
| | | | | | (4-(3,3-dimethylureido)benzyl)-1-methyl- |
| | | | | | 4,5,6,7-tetrahydro-1H-indazole-3- |
| | | | | | carboxamide |
| 27 | 0.006 | 2 | 0.091 | 2 | N-(4-(3,3-dimethylureido)benzyl)-1- |
| | | | | | isopropyl-5-(5-(1-methyl-1H-1,2,4-triazol-5- |
| | | | | | yl)pyridin-2-yl)-4,5,6,7-tetrahydro-1H- |
| | | | | | pyrazolo[4,3-c]pyridine-3-carboxamide |
| 28 | 0.046 | 2 | 0.313 | 5 | 5-(5-(dimethylcarbamoyl)pyridin-2-yl)-N-(4- |
| | | | | | (3,3-dimethylureido)benzyl)-4,5,6,7- |
| | | | | | tetrahydroisoxazolo[4,5-c]pyridine-3- |
| | | | | | carboxamide |
| 29 | 2.230 | 2 | 0.878 | 2 | 5-(5-(dimethylcarbamoyl)pyridin-2-yl)-N-(4- |
| | | | | | (3,3-dimethylureido)benzyl)-4,5,6,7- |
| | | | | | tetrahydropyrazolo[1,5-a]pyrazine-3- |
| | | | | | carboxamide |
| 30 | 0.005 | 2 | 0.087 | 5 | N-(4-(3,3-dimethylureido)benzyl)-1-ethyl-5- |
| | | | | | (5-(1-methyl-1H-imidazol-2-yl)pyridin-2-yl)- |
| | | | | | 4,5,6,7-tetrahydro-1H-pyrazolo[4,3- |
| | | | | | c]pyridine-3-carboxamide |
| 31 | 0.007 | 4 | 0.085 | 7 | 5-(5-(dimethylcarbamoyl)pyridin-2-yl)-N-(4- |
| | | | | | (3,3-dimethylureido)benzyl)-1-(2,2,2- |
| | | | | | trifluoroethyl)-4,5,6,7-tetrahydro-1H- |
| | | | | | pyrazolo[4,3-c]pyridine-3-carboxamide |
| 32 | 0.009 | 2 | 0.040 | 2 | 7-(5-(dimethylcarbamoyl)pyridin-2-yl)-N-(4- |
| | | | | | (3,3-dimethylureido)benzyl)-3-isopropyl- |
| | | | | | 5,6,7,8-tetrahydroimidazo[1,5-a]pyrazine-1- |
| | | | | | carboxamide |
| 33 | 0.002 | 2 | 0.035 | 3 | 5-(5-(6,7-dihydro-5H-pyrrolo[3,4-b]pyridine- |
| | | | | | 6-carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 34 | 0.008 | 2 | 0.071 | 3 | N-(4-(3,3-dimethylureido)benzyl)-1- |
| | | | | | isopropyl-5-(5-(1-methyl-1H-imidazol-2- |
| | | | | | yl)pyrimidin-2-yl)-4,5,6,7-tetrahydro-1H- |
| | | | | | pyrazolo[4,3-c]pyridine-3-carboxamide |
| 35 | 0.009 | 2 | 0.047 | 3 | 5-(5-(dimethylcarbamoyl)pyrimidin-2-yl)-N- |
| | | | | | (4-(3,3-dimethylureido)benzyl)-1-isopropyl- |
| | | | | | 4,5,6,7-tetrahydro-1H-pyrazolo[4,3- |
| | | | | | c]pyridine-3-carboxamide |
| 36 | 0.024 | 3 | 0.254 | 7 | N-(4-(3,3-dimethylureido)benzyl)-1-ethyl-5- |
| | | | | | (5-(2-oxopyrrolidin-1-yl)pyridin-2-yl)-4,5,6,7- |
| | | | | | tetrahydro-1H-pyrazolo[4,3-c]pyridine-3- |
| | | | | | carboxamide |
| 37 | 0.030 | 2 | 0.701 | 6 | 5-(1-acetyl-1,2,3,4-tetrahydroquinolin-6-yl)- |
| | | | | | N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 38 | 0.072 | 2 | 1.636 | 3 | N-(4-(1H-imidazol-2-yl)benzyl)-1-isopropyl- |
| | | | | | 5-(5-(2-oxopyrrolidin-1-yl)pyridin-2-yl)- |
| | | | | | 4,5,6,7-tetrahydro-1H-pyrazolo[4,3- |
| | | | | | c]pyridine-3-carboxamide |
| 39 | 0.748 | 2 | >30.000 | 1 | N-(4-(3,3-dimethylureido)benzyl)-5-(5-((4- |
| | | | | | (3,3- |
| | | | | | dimethylureido)benzyl)carbamoyl)pyridin-2- |
| | | | | | yl)-1-isopropyl-4,5,6,7-tetrahydro-1H- |
| | | | | | pyrazolo[4,3-c]pyridine-3-carboxamide |
| 40 | 0.038 | 2 | 0.324 | 2 | N-(4-(4H-1,2,4-triazol-3-yl)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 41 | 0.003 | 2 | 0.093 | 3 | N-(4-(3,3-dimethylureido)benzyl)-1- |
| | | | | | isopropyl-5-(5-(5-methyl-1H-pyrazol-1- |
| | | | | | yl)pyridin-2-yl)-4,5,6,7-tetrahydro-1H- |
| | | | | | pyrazolo[4,3-c]pyridine-3-carboxamide |
| 42 | 0.010 | 2 | 0.136 | 2 | N-(4-(3,3-dimethylureido)benzyl)-7-(5-(1- |
| | | | | | methyl-1H-imidazol-2-yl)pyridin-2-yl)-3- |
| | | | | | (trifluoromethyl)-5,6,7,8- |
| | | | | | tetrahydroimidazo[1,5-a]pyrazine-1- |
| | | | | | carboxamide |
| 43 | 0.286 | 2 | 1.305 | 2 | 5-(5-(dimethylcarbamoyl)pyridin-2-yl)-1- |
| | | | | | isopropyl-N-(4-(6-oxo-1,6-dihydropyrimidin- |
| | | | | | 2-yl)benzyl)-4,5,6,7-tetrahydro-1H- |
| | | | | | pyrazolo[4,3-c]pyridine-3-carboxamide |
| 44 | 0.503 | 2 | 5.402 | 2 | 5-(5-(dimethylcarbamoyl)pyridin-2-yl)-1- |
| | | | | | isopropyl-N-(4-(6-oxo-1,6-dihydropyridin-2- |
| | | | | | yl)benzyl)-4,5,6,7-tetrahydro-1H- |
| | | | | | pyrazolo[4,3-c]pyridine-3-carboxamide |
| 45 | 0.004 | 2 | 0.050 | 2 | N-(4-(3,3-dimethylureido)benzyl)-1- |
| | | | | | isopropyl-5-(5-(1-methyl-1H-imidazol-2- |
| | | | | | yl)pyridin-2-yl)-4,5,6,7-tetrahydro-1H- |
| | | | | | pyrazolo[4,3-c]pyridine-3-carboxamide |
| 46 | 0.009 | 2 | 0.110 | 3 | 1-cyclopropyl-N-(4-(3,3- |
| | | | | | dimethylureido)benzyl)-5-(5-(1-methyl-1H- |
| | | | | | imidazol-2-yl)pyridin-2-yl)-4,5,6,7- |
| | | | | | tetrahydro-1H-pyrazolo[4,3-c]pyridine-3- |
| | | | | | carboxamide |
| 47 | 1.177 | 2 | >30.000 | 1 | N-(4-(3,3-dimethylureido)benzyl)-1- |
| | | | | | isopropyl-5-(5-(3-methyl-1H-pyrazol-1- |
| | | | | | yl)pyridin-2-yl)-4,5,6,7-tetrahydro-1H- |
| | | | | | pyrazolo[4,3-c]pyridine-3-carboxamide |
| 48 | 0.952 | 2 | 3.957 | 5 | 5-(5-(dimethylcarbamoyl)pyridin-2-yl)-N-(4- |
| | | | | | (methylcarbamoyl)benzyl)-4,5,6,7- |
| | | | | | tetrahydrothieno[3,2-c]pyridine-3- |
| | | | | | carboxamide |
| 49 | 0.009 | 2 | 0.039 | 2 | rac-(R)-5-(5-(dimethylcarbamoyl)pyridin-2- |
| | | | | | yl)-N-(4-(3,3-dimethylureido)benzyl)-1,6- |
| | | | | | dimethyl-4,5,6,7-tetrahydro-1H- |
| | | | | | pyrazolo[4,3-c]pyridine-3-carboxamide |
| 50 | 0.035 | 2 | 0.151 | 1 | (R)-5-(5-(dimethylcarbamoyl)pyridin-2-yl)- |
| | | | | | N-(4-(3,3-dimethylureido)benzyl)-1,6- |
| | | | | | dimethyl-4,5,6,7-tetrahydro-1H- |
| | | | | | pyrazolo[4,3-c]pyridine-3-carboxamide |
| 51 | 0.004 | 2 | 0.030 | 2 | (S)-5-(5-(dimethylcarbamoyl)pyridin-2-yl)- |
| | | | | | N-(4-(3,3-dimethylureido)benzyl)-1,6- |
| | | | | | dimethyl-4,5,6,7-tetrahydro-1H- |
| | | | | | pyrazolo[4,3-c]pyridine-3-carboxamide |
| 52 | 0.009 | 2 | 0.064 | 2 | 3-(1,1-difluoroethyl)-7-(5- |
| | | | | | (dimethylcarbamoyl)pyridin-2-yl)-N-(4-(3,3- |
| | | | | | dimethylureido)benzyl)-5,6,7,8- |
| | | | | | tetrahydroimidazo[1,5-a]pyrazine-1- |
| | | | | | carboxamide |
| 53 | 0.008 | 2 | 0.019 | 3 | rel-(R or S)-7-(5- |
| | | | | | (dimethylcarbamoyl)pyridin-2-yl)-N-(4-(3,3- |
| | | | | | dimethylureido)benzyl)-3,5-dimethyl- |
| | | | | | 5,6,7,8-tetrahydroimidazo[1,5-a]pyrazine-1- |
| | | | | | carboxamide |
| 54 | 0.120 | 2 | 0.332 | 3 | rel-(R or S)-7-(5- |
| | | | | | (dimethylcarbamoyl)pyridin-2-yl)-N-(4-(3,3- |
| | | | | | dimethylureido)benzyl)-3,5-dimethyl- |
| | | | | | 5,6,7,8-tetrahydroimidazo[1,5-a]pyrazine-1- |
| | | | | | carboxamide |
| 55 | 0.010 | 3 | 0.012 | 1 | rac-(R)-5-(5-(dimethylcarbamoyl)pyridin-2- |
| | | | | | yl)-N-(4-(3,3-dimethylureido)benzyl)-1,4- |
| | | | | | dimethyl-4,5,6,7-tetrahydro-1H- |
| | | | | | pyrazolo[4,3-c]pyridine-3-carboxamide |
| 56 | 0.013 | 2 | 0.369 | 1 | N-(4-(3,3-dimethylureido)benzyl)-1- |
| | | | | | isopropyl-5-(5-(2-methyl-5-oxopyrrolidin-1- |
| | | | | | yl)pyridin-2-yl)-4,5,6,7-tetrahydro-1H- |
| | | | | | pyrazolo[4,3-c]pyridine-3-carboxamide |
| 57 | 0.098 | 2 | 0.182 | 2 | rac-(R)-5-(5-(dimethylcarbamoyl)pyridin-2- |
| | | | | | yl)-N-(4-(3,3-dimethylureido)benzyl)-7- |
| | | | | | ethyl-4,5,6,7-tetrahydro-1H-pyrazolo[4,3- |
| | | | | | c]pyridine-3-carboxamide |
| 58 | 0.024 | 2 | 0.170 | 3 | rel-(R or S)-5-(5- |
| | | | | | (dimethylcarbamoyl)pyridin-2-yl)-N-(4-(3,3- |
| | | | | | dimethylureido)benzyl)-7-ethyl-1-methyl- |
| | | | | | 4,5,6,7-tetrahydro-1H-pyrazolo[4,3- |
| | | | | | c]pyridine-3-carboxamide |
| 59 | 0.003 | 2 | 0.027 | 3 | rac-(R)-N-(4-(3,3-dimethylureido)benzyl)- |
| | | | | | 1,7-dimethyl-5-(5-(1-methyl-1H-imidazol-2- |
| | | | | | yl)pyridin-2-yl)-4,5,6,7-tetrahydro-1H- |
| | | | | | pyrazolo[4,3-c]pyridine-3-carboxamide |
| 60 | 0.032 | 2 | 0.910 | 1 | N-(4-(3,3-dimethylureido)benzyl)-1- |
| | | | | | isopropyl-5-(5-(3-methyl-2-oxoimidazolidin- |
| | | | | | 1-yl)pyridin-2-yl)-4,5,6,7-tetrahydro-1H- |
| | | | | | pyrazolo[4,3-c]pyridine-3-carboxamide |
| 61 | 0.075 | 2 | 2.586 | 1 | N-(4-(3,3-dimethylureido)benzyl)-1- |
| | | | | | isopropyl-5-(5-(3-methyl-2-oxopyrrolidin-1- |
| | | | | | yl)pyridin-2-yl)-4,5,6,7-tetrahydro-1H- |
| | | | | | pyrazolo[4,3-c]pyridine-3-carboxamide |
| 62 | 0.009 | 2 | 0.066 | 2 | rac-(R)-1-(1-cyclopropylethyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 63 | 0.014 | 2 | 0.105 | 2 | (R)-5-(5-(dimethylcarbamoyl)pyridin-2-yl)- |
| | | | | | N-(4-(3,3-dimethylureido)benzyl)-1-ethyl-7- |
| | | | | | methyl-4,5,6,7-tetrahydro-1H-pyrazolo[4,3- |
| | | | | | c]pyridine-3-carboxamide |
| 64 | 0.004 | 2 | 0.073 | 2 | 5-(5-(1H-1,2,3-triazol-1-yl)pyridin-2-yl)-N- |
| | | | | | (4-(3,3-dimethylureido)benzyl)-1-isopropyl- |
| | | | | | 4,5,6,7-tetrahydro-1H-pyrazolo[4,3- |
| | | | | | c]pyridine-3-carboxamide |
| 65 | 0.208 | 2 | 28.130 | 1 | N-(4-(3,3-dimethylureido)benzyl)-1- |
| | | | | | isopropyl-5-(5-(4-oxo-5- |
| | | | | | azaspiro[2.4]heptan-5-yl)pyridin-2-yl)- |
| | | | | | 4,5,6,7-tetrahydro-1H-pyrazolo[4,3- |
| | | | | | c]pyridine-3-carboxamide |
| 66 | 0.013 | 2 | 0.089 | 1 | (S)-N-(4-(3,3-dimethylureido)benzyl)-1- |
| | | | | | isopropyl-5-(5-(4-methyl-2-oxopyrrolidin-1- |
| | | | | | yl)pyridin-2-yl)-4,5,6,7-tetrahydro-1H- |
| | | | | | pyrazolo[4,3-c]pyridine-3-carboxamide |
| 67 | 0.064 | 2 | 5.021 | 1 | (R)-N-(4-(3,3-dimethylureido)benzyl)-1- |
| | | | | | isopropyl-5-(5-(4-methyl-2-oxopyrrolidin-1- |
| | | | | | yl)pyridin-2-yl)-4,5,6,7-tetrahydro-1H- |
| | | | | | pyrazolo[4,3-c]pyridine-3-carboxamide |
| 68 | 0.009 | 2 | 0.043 | 2 | rac-(4R,8S)-10-(5- |
| | | | | | (dimethylcarbamoyl)pyridin-2-yl)-N-(4-(3,3- |
| | | | | | dimethylureido)benzyl)-1-methyl- |
| | | | | | 4,5,6,7,8,9-hexahydro-1H-4,8- |
| | | | | | epiminocycloocta[c]pyrazole-3- |
| | | | | | carboxamide |
| 69 | 1.074 | 2 | 8.108 | 1 | (S)-N-(4-(3,3-dimethylureido)benzyl)-5-(5- |
| | | | | | (2-(hydroxymethyl)-5-oxopyrrolidin-1- |
| | | | | | yl)pyridin-2-yl)-1-isopropyl-4,5,6,7- |
| | | | | | tetrahydro-1H-pyrazolo[4,3-c]pyridine-3- |
| | | | | | carboxamide |
| 70 | 0.008 | 2 | 0.081 | 3 | 5-(5-(dimethylcarbamoyl)pyridin-2-yl)-N-(4- |
| | | | | | (3,3-dimethylureido)benzyl)-7,7-difluoro-1- |
| | | | | | methyl-4,5,6,7-tetrahydro-1H-pyrazolo[4,3- |
| | | | | | c]pyridine-3-carboxamide |
| 71 | 0.002 | 2 | 0.014 | 3 | rac-(R)-5-(5-(dimethylcarbamoyl)pyridin-2- |
| | | | | | yl)-N-(4-(3,3-dimethylureido)benzyl)-1- |
| | | | | | ethyl-7-methyl-4,5,6,7-tetrahydro-1H- |
| | | | | | pyrazolo[4,3-c]pyridine-3-carboxamide |
| 72 | 0.308 | 2 | 1.104 | 3 | rac-(4R,8S)-10-(5- |
| | | | | | (dimethylcarbamoyl)pyridin-2-yl)-N-(4-(3,3- |
| | | | | | dimethylureido)benzyl)-1-methyl- |
| | | | | | 1,4,5,7,8,9-hexahydro-4,8- |
| | | | | | epiminooxocino[5,4-c]pyrazole-3- |
| | | | | | carboxamide |
| 73 | 0.121 | 2 | 9.348 | 1 | N-(4-(3,3-dimethylureido)benzyl)-1- |
| | | | | | isopropyl-5-(5-(5-oxo-4- |
| | | | | | azaspiro[2.4]heptan-4-yl)pyridin-2-yl)- |
| | | | | | 4,5,6,7-tetrahydro-1H-pyrazolo[4,3- |
| | | | | | c]pyridine-3-carboxamide |
| 74 | 0.011 | 2 | 0.120 | 2 | 5-(5-(dimethylcarbamoyl)pyridin-2-yl)-N-(4- |
| | | | | | (3,3-dimethylureido)benzyl)-1-propyl- |
| | | | | | 4,5,6,7-tetrahydro-1H-pyrazolo[4,3- |
| | | | | | c]pyridine-3-carboxamide |
| 75 | 2.829 | 2 | 4.558 | 1 | (R)-N-(4-(3,3-dimethylureido)benzyl)-5-(5- |
| | | | | | (4-(hydroxymethyl)-2-oxopyrrolidin-1- |
| | | | | | yl)pyridin-2-yl)-1-isopropyl-4,5,6,7- |
| | | | | | tetrahydro-1H-pyrazolo[4,3-c]pyridine-3- |
| | | | | | carboxamide |
| 76 | 0.057 | 2 | 0.156 | 2 | 5-(5-(dimethylcarbamoyl)pyridin-2-yl)-N-(4- |
| | | | | | (3,3-dimethylureido)benzyl)-1-(tetrahydro- |
| | | | | | 2H-pyran-4-yl)-4,5,6,7-tetrahydro-1H- |
| | | | | | pyrazolo[4,3-c]pyridine-3-carboxamide |
| 77 | 0.004 | 2 | 0.045 | 3 | rac-(R)-5-(5-(dimethylcarbamoyl)pyridin-2- |
| | | | | | yl)-N-(4-(3,3-dimethylureido)benzyl)-7- |
| | | | | | ethyl-1-methyl-4,5,6,7-tetrahydro-1H- |
| | | | | | pyrazolo[4,3-c]pyridine-3-carboxamide |
| 78 | 0.003 | 2 | 0.014 | 3 | rel-(R or S)-5-(5- |
| | | | | | (dimethylcarbamoyl)pyridin-2-yl)-N-(4-(3,3- |
| | | | | | dimethylureido)benzyl)-7-ethyl-1-methyl- |
| | | | | | 4,5,6,7-tetrahydro-1H-pyrazolo[4,3- |
| | | | | | c]pyridine-3-carboxamide |
| 79 | 0.041 | 2 | 1.778 | 1 | (S)-N-(4-(3,3-dimethylureido)benzyl)-5-(5- |
| | | | | | (2-ethyl-5-oxopyrrolidin-1-yl)pyridin-2-yl)-1- |
| | | | | | isopropyl-4,5,6,7-tetrahydro-1H- |
| | | | | | pyrazolo[4,3-c]pyridine-3-carboxamide |
| 80 | >10.000 | 1 | >30.000 | 1 | rac-(R)-N-(4-(3,3-dimethylureido)benzyl)-5- |
| | | | | | (5-(5-(hydroxymethyl)-2-oxooxazolidin-3- |
| | | | | | yl)pyridin-2-yl)-1-isopropyl-4,5,6,7- |
| | | | | | tetrahydro-1H-pyrazolo[4,3-c]pyridine-3- |
| | | | | | carboxamide |
| 81 | 0.034 | 2 | 0.115 | 3 | rac-(R)-5-(5-(dimethylcarbamoyl)pyridin-2- |
| | | | | | yl)-N-(1-(4-(3,3- |
| | | | | | dimethylureido)phenyl)ethyl)-1-isopropyl- |
| | | | | | 4,5,6,7-tetrahydro-1H-pyrazolo[4,3- |
| | | | | | c]pyridine-3-carboxamide |
| 82 | 0.101 | 2 | 0.112 | 3 | rel-(R or S)-5-(5- |
| | | | | | (dimethylcarbamoyl)pyridin-2-yl)-N-(4-(3,3- |
| | | | | | dimethylureido)benzyl)-7-ethyl-4,5,6,7- |
| | | | | | tetrahydro-1H-pyrazolo[4,3-c]pyridine-3- |
| | | | | | carboxamide |
| 83 | 0.086 | 2 | 0.278 | 2 | rel-(R or S)-5-(5- |
| | | | | | (dimethylcarbamoyl)pyridin-2-yl)-N-(4-(3,3- |
| | | | | | dimethylureido)benzyl)-7-ethyl-4,5,6,7- |
| | | | | | tetrahydro-1H-pyrazolo[4,3-c]pyridine-3- |
| | | | | | carboxamide |
| 84 | 0.002 | 2 | 0.056 | 1 | (S)-N-(4-(3,3-dimethylureido)benzyl)-5-(5- |
| | | | | | (3-fluoro-2-oxopyrrolidin-1-yl)pyridin-2-yl)-1- |
| | | | | | isopropyl-4,5,6,7-tetrahydro-1H- |
| | | | | | pyrazolo[4,3-c]pyridine-3-carboxamide |
| 85 | 0.006 | 2 | 0.090 | 3 | rac-(R)-1-(sec-butyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 86 | >10.000 | 1 | >30.000 | 1 | rac-(4R,7S)-9-(5- |
| | | | | | (dimethylcarbamoyl)pyridin-2-yl)-N-(4-(3,3- |
| | | | | | dimethylureido)benzyl)-1-methyl- |
| | | | | | 1,4,5,6,7,8-hexahydro-4,7- |
| | | | | | epiminocyclohepta[c]pyrazole-3- |
| | | | | | carboxamide |
| 87 | 0.015 | 2 | 0.118 | 1 | rel-(R or S)-1-(1-cyclopropylethyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 88 | 0.424 | 2 | 2.078 | 1 | (R)-N-(4-(3,3-dimethylureido)benzyl)-5-(5- |
| | | | | | (2-(hydroxymethyl)-5-oxopyrrolidin-1- |
| | | | | | yl)pyridin-2-yl)-1-isopropyl-4,5,6,7- |
| | | | | | tetrahydro-1H-pyrazolo[4,3-c]pyridine-3- |
| | | | | | carboxamide |
| 89 | 0.012 | 2 | 0.149 | 2 | 1-(cyclopropylmethyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 90 | 0.025 | 2 | 0.067 | 2 | rac-(R)-5-(5-(dimethylcarbamoyl)pyridin-2- |
| | | | | | yl)-N-(4-(3,3-dimethylureido)benzyl)-1- |
| | | | | | (tetrahydrofuran-3-yl)-4,5,6,7-tetrahydro- |
| | | | | | 1H-pyrazolo[4,3-c]pyridine-3-carboxamide |
| 91 | 0.014 | 2 | 0.327 | 1 | N-(4-(3,3-dimethylureido)benzyl)-1- |
| | | | | | isopropyl-5-(5-(2-oxo-3- |
| | | | | | azabicyclo[3.1.0]hexan-3-yl)pyridin-2-yl)- |
| | | | | | 4,5,6,7-tetrahydro-1H-pyrazolo[4,3- |
| | | | | | c]pyridine-3-carboxamide |
| 92 | 0.020 | 2 | 0.062 | 2 | rac-(R)-5-(5-(dimethylcarbamoyl)pyridin-2- |
| | | | | | yl)-N-(4-(3,3-dimethylureido)benzyl)-1-(1- |
| | | | | | methoxypropan-2-yl)-4,5,6,7-tetrahydro- |
| | | | | | 1H-pyrazolo[4,3-c]pyridine-3-carboxamide |
| 93 | 0.525 | 2 | 2.270 | 1 | rac-(R)-N-(4-(3,3-dimethylureido)benzyl)-5- |
| | | | | | (5-(4-hydroxy-2-oxopyrrolidin-1-yl)pyridin-2- |
| | | | | | yl)-1-isopropyl-4,5,6,7-tetrahydro-1H- |
| | | | | | pyrazolo[4,3-c]pyridine-3-carboxamide |
| 94 | 0.049 | 2 | 0.089 | 3 | 5-(5-(dimethylcarbamoyl)pyridin-2-yl)-N-(4- |
| | | | | | (3,3-dimethylureido)benzyl)-1-(oxetan-3-yl)- |
| | | | | | 4,5,6,7-tetrahydro-1H-pyrazolo[4,3- |
| | | | | | c]pyridine-3-carboxamide |
| 95 | 0.001 | 2 | 0.006 | 6 | rac-(R)-5-(5-(dimethylcarbamoyl)pyridin-2- |
| | | | | | yl)-N-(4-(3,3-dimethylureido)benzyl)-1- |
| | | | | | isopropyl-7-methyl-4,5,6,7-tetrahydro-1H- |
| | | | | | pyrazolo[4,3-c]pyridine-3-carboxamide |
| 96 | 0.005 | 2 | 0.066 | 3 | 1-cyclobutyl-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 97 | 0.065 | 2 | 3.483 | 1 | N-(4-(3,3-dimethylureido)benzyl)-1- |
| | | | | | isopropyl-5-(5-(6-oxo-5- |
| | | | | | azaspiro[2.4]heptan-5-yl)pyridin-2-yl)- |
| | | | | | 4,5,6,7-tetrahydro-1H-pyrazolo[4,3- |
| | | | | | c]pyridine-3-carboxamide |
| 98 | 0.048 | 2 | 0.573 | 1 | (R)-N-(4-(3,3-dimethylureido)benzyl)-5-(5- |
| | | | | | (3-fluoro-2-oxopyrrolidin-1-yl)pyridin-2-yl)-1- |
| | | | | | isopropyl-4,5,6,7-tetrahydro-1H- |
| | | | | | pyrazolo[4,3-c]pyridine-3-carboxamide |
| 99 | 0.017 | 2 | 0.453 | 1 | N-(4-(3,3-dimethylureido)benzyl)-1- |
| | | | | | isopropyl-5-(5-(4-methyl-2-oxooxazolidin-3- |
| | | | | | yl)pyridin-2-yl)-4,5,6,7-tetrahydro-1H- |
| | | | | | pyrazolo[4,3-c]pyridine-3-carboxamide |
| 100 | 0.174 | 2 | 1.796 | 1 | N-(4-(3,3-dimethylureido)benzyl)-1- |
| | | | | | isopropyl-5-(5-(5-oxo-1,4-oxazepan-4- |
| | | | | | yl)pyridin-2-yl)-4,5,6,7-tetrahydro-1H- |
| | | | | | pyrazolo[4,3-c]pyridine-3-carboxamide |
| 101 | 0.005 | 2 | 0.053 | 2 | rac-(R)-5-(5-(dimethylcarbamoyl)pyridin-2- |
| | | | | | yl)-N-(4-(3,3-dimethylureido)benzyl)-1- |
| | | | | | (1,1,1-trifluoropropan-2-yl)-4,5,6,7- |
| | | | | | tetrahydro-1H-pyrazolo[4,3-c]pyridine-3- |
| | | | | | carboxamide |
| 103 | 0.047 | 2 | 0.535 | 1 | N-(4-(3,3-dimethylureido)benzyl)-1- |
| | | | | | isopropyl-5-(5-(3-oxomorpholino)pyridin-2- |
| | | | | | yl)-4,5,6,7-tetrahydro-1H-pyrazolo[4,3- |
| | | | | | c]pyridine-3-carboxamide |
| 104 | 0.007 | 2 | 0.107 | 3 | 1-(bicyclo[1.1.1]pentan-1-yl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 105 | 0.003 | 2 | 0.021 | 2 | 5-(5-(dimethylcarbamoyl)pyridin-2-yl)-N-(4- |
| | | | | | (3,3-dimethylureido)benzyl)-1,7,7-trimethyl- |
| | | | | | 4,5,6,7-tetrahydro-1H-pyrazolo[4,3- |
| | | | | | c]pyridine-3-carboxamide |
| 106 | 0.008 | 2 | 0.049 | 1 | 5′-(5-(dimethylcarbamoyl)pyridin-2-yl)-N-(4- |
| | | | | | (3,3-dimethylureido)benzyl)-1′-methyl- |
| | | | | | 1′,4′,5′,6′-tetrahydrospiro[cyclopropane-1,7′- |
| | | | | | pyrazolo[4,3-c]pyridine]-3′-carboxamide |
| 107 | 0.013 | 2 | 0.219 | 1 | N-(4-(3,3-dimethylureido)benzyl)-1- |
| | | | | | isopropyl-5-(5-(2-oxooxazolidin-3-yl)pyridin- |
| | | | | | 2-yl)-4,5,6,7-tetrahydro-1H-pyrazolo[4,3- |
| | | | | | c]pyridine-3-carboxamide |
| 108 | 0.030 | 2 | 0.536 | 4 | N-(4-(3,3-dimethylureido)benzyl)-1- |
| | | | | | isopropyl-5-(2-oxo-2H-[1,3′-bipyridin]-6′-yl)- |
| | | | | | 4,5,6,7-tetrahydro-1H-pyrazolo[4,3- |
| | | | | | c]pyridine-3-carboxamide |
| 109 | 0.117 | 2 | >30.000 | 1 | N-(4-(3,3-dimethylureido)benzyl)-1- |
| | | | | | isopropyl-5-(5-(3-oxo-2- |
| | | | | | azabicyclo[2.2.1]heptan-2-yl)pyridin-2-yl)- |
| | | | | | 4,5,6,7-tetrahydro-1H-pyrazolo[4,3- |
| | | | | | c]pyridine-3-carboxamide |
| 110 | 0.523 | 2 | 2.089 | 1 | N-(4-(3,3-dimethylureido)benzyl)-5-(5-(4- |
| | | | | | (hydroxymethyl)-2-oxooxazolidin-3- |
| | | | | | yl)pyridin-2-yl)-1-isopropyl-4,5,6,7- |
| | | | | | tetrahydro-1H-pyrazolo[4,3-c]pyridine-3- |
| | | | | | carboxamide |
| 111 | 0.003 | 4 | 0.013 | 4 | (S)-5-(5-(dimethylcarbamoyl)pyridin-2-yl)- |
| | | | | | N-(4-(3,3-dimethylureido)benzyl)-1-ethyl-6- |
| | | | | | methyl-4,5,6,7-tetrahydro-1H-pyrazolo[4,3- |
| | | | | | c]pyridine-3-carboxamide |
| 112 | 0.015 | 2 | 0.078 | 4 | (R)-5-(5-(dimethylcarbamoyl)pyridin-2-yl)- |
| | | | | | N-(4-(3,3-dimethylureido)benzyl)-1- |
| | | | | | isopropyl-7-methyl-4,5,6,7-tetrahydro-1H- |
| | | | | | pyrazolo[4,3-c]pyridine-3-carboxamide |
| 113 | 0.017 | 3 | 0.161 | 4 | (R)-5-(5-(dimethylcarbamoyl)pyridin-2-yl)- |
| | | | | | N-(4-(3,3-dimethylureido)benzyl)-1-ethyl-6- |
| | | | | | methyl-4,5,6,7-tetrahydro-1H-pyrazolo[4,3- |
| | | | | | c]pyridine-3-carboxamide |
| 114 | 0.020 | 3 | 0.211 | 4 | (R)-5-(5-(dimethylcarbamoyl)pyridin-2-yl)- |
| | | | | | N-(4-(3,3-dimethylureido)benzyl)-1- |
| | | | | | isopropyl-6-methyl-4,5,6,7-tetrahydro-1H- |
| | | | | | pyrazolo[4,3-c]pyridine-3-carboxamide |
| 115 | 0.002 | 2 | 0.012 | 2 | rac-(R)-5-(5-(dimethylcarbamoyl)pyridin-2- |
| | | | | | yl)-N-(4-(3,3-dimethylureido)benzyl)-1,7- |
| | | | | | diethyl-4,5,6,7-tetrahydro-1H-pyrazolo[4,3- |
| | | | | | c]pyridine-3-carboxamide |
| 116 | 0.010 | 2 | 0.061 | 1 | rac-(R)-5-(5-(dimethylcarbamoyl)pyridin-2- |
| | | | | | yl)-N-(4-(3,3-dimethylureido)benzyl)-1,6- |
| | | | | | diethyl-4,5,6,7-tetrahydro-1H-pyrazolo[4,3- |
| | | | | | c]pyridine-3-carboxamide |
| 117 | 0.003 | 2 | 0.013 | 2 | rel-(R or S)-5-(5- |
| | | | | | (dimethylcarbamoyl)pyridin-2-yl)-N-(4-(3,3- |
| | | | | | dimethylureido)benzyl)-1,6-diethyl-4,5,6,7- |
| | | | | | tetrahydro-1H-pyrazolo[4,3-c]pyridine-3- |
| | | | | | carboxamide |
| 118 | 0.025 | 2 | 0.116 | 1 | rel-(R or S)-5-(5- |
| | | | | | (dimethylcarbamoyl)pyridin-2-yl)-N-(4-(3,3- |
| | | | | | dimethylureido)benzyl)-1,6-diethyl-4,5,6,7- |
| | | | | | tetrahydro-1H-pyrazolo[4,3-c]pyridine-3- |
| | | | | | carboxamide |
| 119 | 0.0003 | 2 | 0.004 | 4 | (S)-1-cyclobutyl-5-(5- |
| | | | | | (dimethylcarbamoyl)pyridin-2-yl)-N-(4-(3,3- |
| | | | | | dimethylureido)benzyl)-7-methyl-4,5,6,7- |
| | | | | | tetrahydro-1H-pyrazolo[4,3-c]pyridine-3- |
| | | | | | carboxamide |
| 120 | 0.001 | 2 | 0.004 | 4 | (S)-1-(cyclopropylmethyl)-5-(5- |
| | | | | | (dimethylcarbamoyl)pyrimidin-2-yl)-N-(4- |
| | | | | | (3,3-dimethylureido)benzyl)-7-methyl- |
| | | | | | 4,5,6,7-tetrahydro-1H-pyrazolo[4,3- |
| | | | | | c]pyridine-3-carboxamide |
| 121 | 0.001 | 2 | 0.006 | 5 | rac-(R)-5-(5-(dimethylcarbamoyl)pyridin-2- |
| | | | | | yl)-N-((5-(3,3-dimethylureido)pyridin-2- |
| | | | | | yl)methyl)-1-isopropyl-7-(trifluoromethyl)- |
| | | | | | 4,5,6,7-tetrahydro-1H-pyrazolo[4,3- |
| | | | | | c]pyridine-3-carboxamide |
| 122 | 0.024 | 2 | 0.900 | 4 | (R)-N-(4-(3,3-dimethylureido)benzyl)-1,6- |
| | | | | | dimethyl-5-(5-(1-methyl-1H-imidazol-2- |
| | | | | | yl)pyridin-2-yl)-4,5,6,7-tetrahydro-1H- |
| | | | | | pyrazolo[4,3-c]pyridine-3-carboxamide |
| 123 | 0.041 | 2 | 1.012 | 3 | rel-(R or S)-N-(4-(1H-imidazol-2-yl)benzyl)- |
| | | | | | 5-(5-(dimethylcarbamoyl)pyridin-2-yl)-1- |
| | | | | | isopropyl-7-(trifluoromethyl)-4,5,6,7- |
| | | | | | tetrahydro-1H-pyrazolo[4,3-c]pyridine-3- |
| | | | | | carboxamide |
| 124 | 0.0003 | 2 | 0.005 | 5 | rel-(R or S)-N-(4-(1H-imidazol-2-yl)benzyl)- |
| | | | | | 5-(5-(dimethylcarbamoyl)pyridin-2-yl)-1- |
| | | | | | isopropyl-7-(trifluoromethyl)-4,5,6,7- |
| | | | | | tetrahydro-1H-pyrazolo[4,3-c]pyridine-3- |
| | | | | | carboxamide |
| 125 | 0.001 | 4 | 0.004 | 4 | (S)-5-(5-(dimethylcarbamoyl)pyrimidin-2- |
| | | | | | yl)-N-(4-(3,3-dimethylureido)benzyl)-1- |
| | | | | | ethyl-7-methyl-4,5,6,7-tetrahydro-1H- |
| | | | | | pyrazolo[4,3-c]pyridine-3-carboxamide |
| 126 | 0.001 | 3 | 0.005 | 4 | (S)-5-(5-(dimethylcarbamoyl)pyridin-2-yl)- |
| | | | | | N-(4-(3,3-dimethylureido)benzyl)-1-ethyl-7- |
| | | | | | methyl-4,5,6,7-tetrahydro-1H-pyrazolo[4,3- |
| | | | | | c]pyridine-3-carboxamide |
| 127 | 0.001 | 5 | 0.004 | 4 | (S)-5-(5-dimethylcarbamoyl)pyrimidin-2-yl)- |
| | | | | | N-(4-(3,3-dimethylureido)benzyl)-1- |
| | | | | | isopropyl-7-methyl-4,5,6,7-tetrahydro-1H- |
| | | | | | pyrazolo[4,3-c]pyridine-3-carboxamide |
| 128 | 0.0003 | 5 | 0.003 | 8 | (S)-5-(5-(dimethylcarbamoyl)pyridin-2-yl)- |
| | | | | | N-(4-(3,3-dimethylureido)benzyl)-1- |
| | | | | | isopropyl-7-methyl-4,5,6,7-tetrahydro-1H- |
| | | | | | pyrazolo[4,3-c]pyridine-3-carboxamide |
| 129 | 0.001 | 2 | 0.009 | 2 | (S)-N-(4-(3,3-dimethylureido)benzyl)-1- |
| | | | | | ethyl-7-methyl-5-(5-(2-oxopyrrolidin-1- |
| | | | | | yl)pyridin-2-yl)-4,5,6,7-tetrahydro-1H- |
| | | | | | pyrazolo[4,3-c]pyridine-3-carboxamide |
| 130 | 0.023 | 2 | 0.077 | 3 | rac-(R)-7-(5-(dimethylcarbamoyl)pyridin-2- |
| | | | | | yl)-N-(4-(3,3-dimethylureido)benzyl)-5- |
| | | | | | ethyl-3-methyl-5,6,7,8- |
| | | | | | tetrahydroimidazo[1,5-a]pyrazine-1- |
| | | | | | carboxamide |
| 131 | 0.103 | 2 | 0.256 | 3 | rel-(R or S)-7-(5- |
| | | | | | (dimethylcarbamoyl)pyridin-2-yl)-N-(4-(3,3- |
| | | | | | dimethylureido)benzyl)-5-ethyl-3-methyl- |
| | | | | | 5,6,7,8-tetrahydroimidazo[1,5-a]pyrazine-1- |
| | | | | | carboxamide |
| 132 | 0.0002 | 2 | 0.0036 | 3 | (S)-5-(4-(dimethylcarbamoyl)phenyl)-1- |
| | | | | | isopropyl-7-methyl-N-(4-(4- |
| | | | | | methylpiperazine-1-carboxamido)benzyl)- |
| | | | | | 4,5,6,7-tetrahydro-1H-pyrazolo[4,3- |
| | | | | | c]pyridine-3-carboxamide |
| 133 | 0.028 | 2 | 0.048 | 3 | 5-(5-(dimethylcarbamoyl)pyridin-2-yl)-1- |
| | | | | | isopropyl-N-(4-ureidobenzyl)-4,5,6,7- |
| | | | | | tetrahydro-1H-pyrazolo[4,3-c]pyridine-3- |
| | | | | | carboxamide |
| 134 | 0.007 | 2 | 0.046 | 3 | 5-(5-(dimethylcarbamoyl)pyridin-2-yl)-1- |
| | | | | | isopropyl-N-(4-(3-methylureido)benzyl)- |
| | | | | | 4,5,6,7-tetrahydro-1H-pyrazolo[4,3- |
| | | | | | c]pyridine-3-carboxamide |
| 135 | 0.136 | 2 | 1.268 | 2 | N-(4-(3,3-dimethylureido)benzyl)-1- |
| | | | | | isopropyl-5-(5-(methylcarbamoyl)pyridin-2- |
| | | | | | yl)-4,5,6,7-tetrahydro-1H-pyrazolo[4,3- |
| | | | | | c]pyridine-3-carboxamide |
| 136 | 0.012 | 4 | 0.196 | 3 | 5-(5-(dimethylcarbamoyl)pyridin-2-yl)-1- |
| | | | | | isopropyl-N-(4-(pyrrolidine-1- |
| | | | | | carboxamido)benzyl)-4,5,6,7-tetrahydro- |
| | | | | | 1H-pyrazolo[4,3-c]pyridine-3-carboxamide |
| 137 | 1.864 | 2 | 9.688 | 2 | 5-(5-carbamoylpyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 138 | 0.248 | 2 | 1.137 | 3 | N-(4-(3,3-dimethylureido)benzyl)-1- |
| | | | | | isopropyl-5-(5-(methylsulfonamido)pyridin- |
| | | | | | 2-yl)-4,5,6,7-tetrahydro-1H-pyrazolo[4,3- |
| | | | | | c]pyridine-3-carboxamide |
| 139 | 0.002 | 5 | 0.007 | 6 | (S)-5-(5-(dimethylcarbamoyl)pyridin-2-yl)- |
| | | | | | N-(4-(3,3-dimethylureido)benzyl)-1- |
| | | | | | isopropyl-6-methyl-4,5,6,7-tetrahydro-1H- |
| | | | | | pyrazolo[4,3-c]pyridine-3-carboxamide |
| 140 | 0.012 | 2 | 0.144 | 1 | N-(4-((3R,4S)-3,4-difluoropyrrolidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 141 | 0.022 | 2 | 0.078 | 1 | N-(4-(3-(dimethylamino)azetidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 142 | 0.015 | 2 | 0.039 | 1 | (1S,4S)-N-(4-((5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3- |
| | | | | | carboxamido)methyl)phenyl)-2-oxa-5- |
| | | | | | azabicyclo[2.2.2]octane-5-carboxamide |
| 143 | 0.011 | 2 | ND | ND | rac-N-(4-((1R,4S)-2- |
| | | | | | azabicyclo[2.2.1]heptane-2- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 144 | 0.007 | 2 | 0.292 | 1 | 5-(5-(dimethylcarbamoyl)pyridin-2-yl)-1- |
| | | | | | isopropyl-N-(4-(piperidine-1- |
| | | | | | carboxamido)benzyl)-4,5,6,7-tetrahydro- |
| | | | | | 1H-pyrazolo[4,3-c]pyridine-3-carboxamide |
| 145 | 0.006 | 2 | ND | ND | rac-(R)-N-(4-(3-(difluoromethyl)pyrrolidine- |
| | | | | | 1-carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 146 | 0.127 | 2 | 2.116 | 3 | N-(4-(3,3-dimethylureido)benzyl)-5-(5-(1,1- |
| | | | | | dioxidoisothiazolidin-2-yl)pyridin-2-yl)-1- |
| | | | | | isopropyl-4,5,6,7-tetrahydro-1H- |
| | | | | | pyrazolo[4,3-c]pyridine-3-carboxamide |
| 147 | 0.026 | 2 | 0.252 | 3 | N-(4-acetamidobenzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 148 | 0.010 | 2 | 0.176 | 3 | 1-(tert-butyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 149 | 0.005 | 1 | ND | ND | N-(4-(4-(cyanomethyl)piperidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 150 | 0.018 | 2 | 0.070 | 2 | N-(4-(3-(dimethylamino)-3-methylazetidine- |
| | | | | | 1-carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 151 | 0.009 | 1 | ND | ND | N-(4-(3-(dimethylamino)piperidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 152 | 0.031 | 2 | 0.155 | 2 | N-(4-((5-(5-(dimethylcarbamoyl)pyridin-2- |
| | | | | | yl)-1-isopropyl-4,5,6,7-tetrahydro-1H- |
| | | | | | pyrazolo[4,3-c]pyridine-3- |
| | | | | | carboxamido)methyl)phenyl)-2-oxa-6- |
| | | | | | azaspiro[3.4]octane-6-carboxamide |
| 153 | 0.011 | 2 | 0.108 | 1 | (S)-5-(5-(dimethylcarbamoyl)pyridin-2-yl)- |
| | | | | | N-(4-(3-fluoropyrrolidine-1- |
| | | | | | carboxamido)benzyl)-1-isopropyl-4,5,6,7- |
| | | | | | tetrahydro-1H-pyrazolo[4,3-c]pyridine-3- |
| | | | | | carboxamide |
| 154 | 0.008 | 2 | 0.449 | 2 | N-(4-(3-(cyclopropylmethyl)ureido)benzyl)- |
| | | | | | 5-(5-(dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 155 | 0.012 | 2 | 0.141 | 1 | rac-(R)-5-(5-(dimethylcarbamoyl)pyridin-2- |
| | | | | | yl)-1-isopropyl-N-(4-(3-((tetrahydrofuran-3- |
| | | | | | yl)methyl)ureido)benzyl)-4,5,6,7-tetrahydro- |
| | | | | | 1H-pyrazolo[4,3-c]pyridine-3-carboxamide |
| 156 | 0.006 | 2 | 0.058 | 1 | N-(4-((5-(5-(dimethylcarbamoyl)pyridin-2- |
| | | | | | yl)-1-isopropyl-4,5,6,7-tetrahydro-1H- |
| | | | | | pyrazolo[4,3-c]pyridine-3- |
| | | | | | carboxamido)methyl)phenyl)-2-oxa-7- |
| | | | | | azaspiro[3.5]nonane-7-carboxamide |
| 157 | 0.008 | 2 | 0.535 | 2 | N-(4-(3-cyclobutylureido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 158 | 0.015 | 2 | 0.130 | 1 | N-(4-(3-cyanoazetidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 159 | 0.016 | 2 | 0.059 | 1 | (S)-N-(4-((5-(5-(dimethylcarbamoyl)pyridin- |
| | | | | | 2-yl)-1-isopropyl-4,5,6,7-tetrahydro-1H- |
| | | | | | pyrazolo[4,3-c]pyridine-3- |
| | | | | | carboxamido)methyl)phenyl)-2- |
| | | | | | (hydroxymethyl)morpholine-4-carboxamide |
| 160 | 0.015 | 2 | 0.021 | 1 | rac-(R)-5-(5-(dimethylcarbamoyl)pyridin-2- |
| | | | | | yl)-N-(4-(3-hydroxy-3-methylpyrrolidine-1- |
| | | | | | carboxamido)benzyl)-1-isopropyl-4,5,6,7- |
| | | | | | tetrahydro-1H-pyrazolo[4,3-c]pyridine-3- |
| | | | | | carboxamide |
| 161 | 0.007 | 2 | 0.199 | 2 | N-(4-(4-(difluoromethyl)piperidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 162 | 0.011 | 2 | 0.066 | 1 | 5-(5-(dimethylcarbamoyl)pyridin-2-yl)-N-(4- |
| | | | | | (4-(2-hydroxyethoxy)piperidine-1- |
| | | | | | carboxamido)benzyl)-1-isopropyl-4,5,6,7- |
| | | | | | tetrahydro-1H-pyrazolo[4,3-c]pyridine-3- |
| | | | | | carboxamide |
| 163 | 0.016 | 2 | 0.030 | 1 | N-(4-(3-amino-3-methylazetidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 164 | 0.013 | 2 | 0.045 | 1 | rac-(R)-N-(4-((5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3- |
| | | | | | carboxamido)methyl)phenyl)-6-oxa-2,9- |
| | | | | | diazaspiro[4.5]decane-2-carboxamide |
| 165 | 0.014 | 2 | 0.156 | 1 | 5-(5-(dimethylcarbamoyl)pyridin-2-yl)-1- |
| | | | | | isopropyl-N-(4-(3-methoxy-3- |
| | | | | | methylazetidine-1-carboxamido)benzyl)- |
| | | | | | 4,5,6,7-tetrahydro-1H-pyrazolo[4,3- |
| | | | | | c]pyridine-3-carboxamide |
| 166 | 0.004 | 2 | 0.021 | 2 | 5-(5-(dimethylcarbamoyl)pyridin-2-yl)-N-(4- |
| | | | | | ((3S,4S)-3-hydroxy-4-methoxypyrrolidine-1- |
| | | | | | carboxamido)benzyl)-1-isopropyl-4,5,6,7- |
| | | | | | tetrahydro-1H-pyrazolo[4,3-c]pyridine-3- |
| | | | | | carboxamide |
| 167 | 0.029 | 2 | 0.154 | 2 | (R)-N-(4-((5-(5-(dimethylcarbamoyl)pyridin- |
| | | | | | 2-yl)-1-isopropyl-4,5,6,7-tetrahydro-1H- |
| | | | | | pyrazolo[4,3-c]pyridine-3- |
| | | | | | carboxamido)methyl)phenyl)-2- |
| | | | | | (hydroxymethyl)morpholine-4-carboxamide |
| 168 | 0.024 | 3 | 0.111 | 2 | 5-(5-(dimethylcarbamoyl)pyridin-2-yl)-1- |
| | | | | | isopropyl-N-(4-(3-methyl-3-(oxetan-3- |
| | | | | | ylmethyl)ureido)benzyl)-4,5,6,7-tetrahydro- |
| | | | | | 1H-pyrazolo[4,3-c]pyridine-3-carboxamide |
| 169 | 0.032 | 3 | 0.125 | 2 | 5-(5-(dimethylcarbamoyl)pyridin-2-yl)-N-(4- |
| | | | | | (3-((1s,3s)-3- |
| | | | | | hydroxycyclobutyl)ureido)benzyl)-1- |
| | | | | | isopropyl-4,5,6,7-tetrahydro-1H- |
| | | | | | pyrazolo[4,3-c/pyridine-3-carboxamide |
| 170 | 0.017 | 2 | 0.082 | 1 | (S)-N-(4-(3-(dimethylamino)pyrrolidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 171 | 0.005 | 2 | 0.057 | 2 | rac-5-(5-(dimethylcarbamoyl)pyridin-2-yl)- |
| | | | | | N-(4-((3R,4R)-3-fluoro-4- |
| | | | | | methoxypyrrolidine-1-carboxamido)benzyl)- |
| | | | | | 1-isopropyl-4,5,6,7-tetrahydro-1H- |
| | | | | | pyrazolo[4,3-c]pyridine-3-carboxamide |
| 172 | 0.011 | 2 | 0.150 | 1 | N-(4-(4-cyano-4-methylpiperidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 173 | 0.011 | 2 | 0.072 | 1 | N-(4-((5-(5-(dimethylcarbamoyl)pyridin-2- |
| | | | | | yl)-1-isopropyl-4,5,6,7-tetrahydro-1H- |
| | | | | | pyrazolo[4,3-c]pyridine-3- |
| | | | | | carboxamido)methyl)phenyl)morpholine-4- |
| | | | | | carboxamide |
| 174 | 0.040 | 2 | 0.212 | 2 | rac-(R)-5-(5-(dimethylcarbamoyl)pyridin-2- |
| | | | | | yl)-N-(4-(2-(hydroxymethyl)pyrrolidine-1- |
| | | | | | carboxamido)benzyl)-1-isopropyl-4,5,6,7- |
| | | | | | tetrahydro-1H-pyrazolo[4,3-c]pyridine-3- |
| | | | | | carboxamide |
| 175 | 0.007 | 2 | 0.067 | 2 | 5-(5-(dimethylcarbamoyl)pyridin-2-yl)-N-(4- |
| | | | | | (4-(hydroxymethyl)piperidine-1- |
| | | | | | carboxamido)benzyl)-1-isopropyl-4,5,6,7- |
| | | | | | tetrahydro-1H-pyrazolo[4,3-c]pyridine-3- |
| | | | | | carboxamide |
| 176 | 0.012 | 2 | 0.051 | 2 | rac-(R)-N-(4-(3-((1,4-dioxan-2-yl)methyl)-3- |
| | | | | | methylureido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 177 | 0.008 | 2 | 0.051 | 2 | 5-(5-(dimethylcarbamoyl)pyridin-2-yl)-1- |
| | | | | | isopropyl-N-(4-(4-methylpiperazine-1- |
| | | | | | carboxamido)benzyl)-4,5,6,7-tetrahydro- |
| | | | | | 1H-pyrazolo[4,3-c]pyridine-3-carboxamide |
| 178 | 0.007 | 2 | 0.045 | 2 | 5-(5-(dimethylcarbamoyl)pyridin-2-yl)-1- |
| | | | | | isopropyl-N-(4-(3-(2- |
| | | | | | methoxyethyl)ureido)benzyl)-4,5,6,7- |
| | | | | | tetrahydro-1H-pyrazolo[4,3-c]pyridine-3- |
| | | | | | carboxamide |
| 179 | 0.007 | 2 | 0.064 | 2 | (R)-5-(5-(dimethylcarbamoyl)pyridin-2-yl)-1- |
| | | | | | isopropyl-N-(4-(3-methoxypyrrolidine-1- |
| | | | | | carboxamido)benzyl)-4,5,6,7-tetrahydro- |
| | | | | | 1H-pyrazolo[4,3-c]pyridine-3-carboxamide |
| 180 | 0.018 | 2 | 0.055 | 2 | 5-(5-(dimethylcarbamoyl)pyridin-2-yl)-N-(4- |
| | | | | | (3-(2-hydroxyethyl)-3-methylureido)benzyl)- |
| | | | | | 1-isopropyl-4,5,6,7-tetrahydro-1H- |
| | | | | | pyrazolo[4,3-c]pyridine-3-carboxamide |
| 181 | 0.260 | 2 | 0.436 | 2 | 5-(5-(dimethylcarbamoyl)pyridin-2-yl)-1- |
| | | | | | isopropyl-N-(4-(3-(oxetan-3- |
| | | | | | ylmethyl)ureido)benzyl)-4,5,6,7-tetrahydro- |
| | | | | | 1H-pyrazolo[4,3-c]pyridine-3-carboxamide |
| 182 | 0.017 | 2 | 0.131 | 2 | (S)-N-(4-(3-cyanopyrrolidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 183 | 0.016 | 2 | 0.067 | 1 | (R)-N-(4-(3-cyanopyrrolidine-1- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 184 | 0.027 | 2 | 0.092 | 2 | N-(4-((5-(5-(dimethylcarbamoyl)pyridin-2- |
| | | | | | yl)-1-isopropyl-4,5,6,7-tetrahydro-1H- |
| | | | | | pyrazolo[4,3-c]pyridine-3- |
| | | | | | carboxamido)methyl)phenyl)-6-oxa-3- |
| | | | | | azabicyclo[3.1.1]heptane-3-carboxamide |
| 185 | 0.024 | 2 | 0.083 | 2 | 5-(5-(dimethylcarbamoyl)pyridin-2-yl)-N-(4- |
| | | | | | (3-hydroxy-3-methylazetidine-1- |
| | | | | | carboxamido)benzyl)-1-isopropyl-4,5,6,7- |
| | | | | | tetrahydro-1H-pyrazolo[4,3-c]pyridine-3- |
| | | | | | carboxamide |
| 186 | 0.010 | 2 | ND | ND | N-(4-(3-azabicyclo[3.1.0]hexane-3- |
| | | | | | carboxamido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 187 | 0.015 | 2 | 0.041 | 1 | (S)-5-(5-(dimethylcarbamoyl)pyridin-2-yl)-1- |
| | | | | | isopropyl-N-(4-(3-methyl-3- |
| | | | | | (tetrahydrofuran-3-yl)ureido)benzyl)- |
| | | | | | 4,5,6,7-tetrahydro-1H-pyrazolo[4,3- |
| | | | | | c]pyridine-3-carboxamide |
| 188 | 0.011 | 2 | 0.069 | 2 | 5-(5-(dimethylcarbamoyl)pyridin-2-yl)-1- |
| | | | | | isopropyl-N-(4-(3-methoxyazetidine-1- |
| | | | | | carboxamido)benzyl)-4,5,6,7-tetrahydro- |
| | | | | | 1H-pyrazolo[4,3-c]pyridine-3-carboxamide |
| 189 | 0.010 | 2 | 0.095 | 1 | N-(4-(3-(2,2-difluoroethyl)-3- |
| | | | | | methylureido)benzyl)-5-(5- |
| | | | | | (dimethylcarbamoyl)pyridin-2-yl)-1- |
| | | | | | isopropyl-4,5,6,7-tetrahydro-1H- |
| | | | | | pyrazolo[4,3-c]pyridine-3-carboxamide |
| 190\hat{ } | 0.020 | 2 | 0.038 | 1 | rel-(R or S)-5-(5- |
| | | | | | (dimethylcarbamoyl)pyridin-2-yl)-N-(4-(3,3- |
| | | | | | dimethylureido)benzyl)-1-(tetrahydrofuran- |
| | | | | | 3-yl)-4,5,6,7-tetrahydro-1H-pyrazolo[4,3- |
| | | | | | c]pyridine-3-carboxamide |
| 191\hat{ } | 0.032 | 2 | 0.126 | 3 | rel-(R or S)-5-(5- |
| | | | | | (dimethylcarbamoyl)pyridin-2-yl)-N-(4-(3,3- |
| | | | | | dimethylureido)benzyl)-1-(tetrahydrofuran- |
| | | | | | 3-yl)-4,5,6,7-tetrahydro-1H-pyrazolo[4,3- |
| | | | | | c]pyridine-3-carboxamide |
| 192 | 1.674 | 2 | 6.445 | 3 | (R)-N-(4-(3,3-dimethylureido)benzyl)-1- |
| | | | | | isopropyl-5-(6-methoxy-5-(3-methyl-2- |
| | | | | | oxopiperidin-3-yl)pyridin-2-yl)-4,5,6,7- |
| | | | | | tetrahydro-1H-pyrazolo[4,3-c]pyridine-3- |
| | | | | | carboxamide |
| 193 | 0.372 | 3 | ND | ND | (S)-5-(5-(3-acetamidopyrrolidine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 194 | 0.401 | 2 | ND | ND | rac-(R)-N-(4-(3,3-dimethylureido)benzyl)-1- |
| | | | | | isopropyl-5-(5-(3- |
| | | | | | (methoxymethyl)pyrrolidine-1- |
| | | | | | carbonyl)pyridin-2-yl)-4,5,6,7-tetrahydro- |
| | | | | | 1H-pyrazolo[4,3-c]pyridine-3-carboxamide |
| 195 | 0.164 | 3 | ND | ND | 5-(5-(1,4-oxazepane-4-carbonyl)pyridin-2- |
| | | | | | yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 196 | 0.090 | 3 | ND | ND | N-(4-(3,3-dimethylureido)benzyl)-1- |
| | | | | | isopropyl-5-(5-(4-propionylpiperazine-1- |
| | | | | | carbonyl)pyridin-2-yl)-4,5,6,7-tetrahydro- |
| | | | | | 1H-pyrazolo[4,3-c]pyridine-3-carboxamide |
| 197 | 0.197 | 3 | ND | ND | (S)-5-(5-(2-carbamoylpyrrolidine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 198 | 0.210 | 2 | ND | ND | rac-(R)-N-(4-(3,3-dimethylureido)benzyl)-5- |
| | | | | | (5-(3-hydroxypyrrolidine-1-carbonyl)pyridin- |
| | | | | | 2-yl)-1-isopropyl-4,5,6,7-tetrahydro-1H- |
| | | | | | pyrazolo[4,3-c]pyridine-3-carboxamide |
| 199 | 0.125 | 3 | ND | ND | N-(4-(3,3-dimethylureido)benzyl)-1- |
| | | | | | isopropyl-5-(5-(4-methoxypiperidine-1- |
| | | | | | carbonyl)pyridin-2-yl)-4,5,6,7-tetrahydro- |
| | | | | | 1H-pyrazolo[4,3-c]pyridine-3-carboxamide |
| 200 | 0.363 | 3 | ND | ND | 5-(5-(4-acetyl-1,4-diazepane-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 201 | 0.343 | 3 | ND | ND | (S)-N-(4-(3,3-dimethylureido)benzyl)-5-(5- |
| | | | | | (3-ethoxypyrrolidine-1-carbonyl)pyridin-2- |
| | | | | | yl)-1-isopropyl-4,5,6,7-tetrahydro-1H- |
| | | | | | pyrazolo[4,3-c]pyridine-3-carboxamide |
| 202 | 8.077 | 1 | ND | ND | (R)-N-(4-(3,3-dimethylureido)benzyl)-1- |
| | | | | | isopropyl-5-(6-methoxy-5-(3-methyl-5- |
| | | | | | oxopyrrolidin-3-yl)pyridin-2-yl)-4,5,6,7- |
| | | | | | tetrahydro-1H-pyrazolo[4,3-c]pyridine-3- |
| | | | | | carboxamide |
| 203\hat{ } | 0.001 | 2 | 0.016 | 2 | rel-(R or S)-N-(4-(3,3- |
| | | | | | dimethylureido)benzyl)-1,7-dimethyl-5-(5- |
| | | | | | (1-methyl-1H-imidazol-2-yl)pyridin-2-yl)- |
| | | | | | 4,5,6,7-tetrahydro-1H-pyrazolo[4,3- |
| | | | | | c]pyridine-3-carboxamide |
| 204\hat{ } | 0.016 | 2 | 0.246 | 2 | rel-(R or S)-N-(4-(3,3- |
| | | | | | dimethylureido)benzyl)-1,7-dimethyl-5-(5- |
| | | | | | (1-methyl-1H-imidazol-2-yl)pyridin-2-yl)- |
| | | | | | 4,5,6,7-tetrahydro-1H-pyrazolo[4,3- |
| | | | | | c]pyridine-3-carboxamide |
| 205 | 0.005 | 2 | 0.019 | 2 | rac-(R)-7-(5-(dimethylcarbamoyl)pyridin-2- |
| | | | | | yl)-N-(4-(3,3-dimethylureido)benzyl)-3- |
| | | | | | ethyl-5-methyl-5,6,7,8- |
| | | | | | tetrahydroimidazo[1,5-a]pyrazine-1- |
| | | | | | carboxamide |
| 206 | 0.097 | 1 | ND | ND | N-(4-(3,3-dimethylureido)benzyl)-1- |
| | | | | | isopropyl-5-(5-(morpholine-4- |
| | | | | | carbonyl)pyridin-2-yl)-4,5,6,7-tetrahydro- |
| | | | | | 1H-pyrazolo[4,3-c]pyridine-3-carboxamide |
| 207 | 0.272 | 3 | ND | ND | 5-(5-((2R,6S)-2,6-dimethylmorpholine-4- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 208 | 0.535 | 3 | ND | ND | rac-(R)-N-(4-(3,3-dimethylureido)benzyl)-5- |
| | | | | | (5-(2-(hydroxymethyl)pyrrolidine-1- |
| | | | | | carbonyl)pyridin-2-yl)-1-isopropyl-4,5,6,7- |
| | | | | | tetrahydro-1H-pyrazolo[4,3-c]pyridine-3- |
| | | | | | carboxamide |
| 209 | 0.692 | 3 | ND | ND | (S)-N-(4-(3,3-dimethylureido)benzyl)-1- |
| | | | | | isopropyl-5-(5-(2- |
| | | | | | (methoxymethyl)pyrrolidine-1- |
| | | | | | carbonyl)pyridin-2-yl)-4,5,6,7-tetrahydro- |
| | | | | | 1H-pyrazolo[4,3-c]pyridine-3-carboxamide |
| 210 | 1.063 | 3 | ND | ND | 5-(5-(3-cyanoazetidine-1-carbonyl)pyridin- |
| | | | | | 2-yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 211 | 0.934 | 3 | ND | ND | (R)-5-(5-(3-acetamidopyrrolidine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 212 | 0.278 | 3 | ND | ND | 5-(5-((1R,5S)-6-oxa-3- |
| | | | | | azabicyclo[3.1.1]heptane-3- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 213 | 0.718 | 3 | ND | ND | N-(4-(3,3-dimethylureido)benzyl)-5-(5- |
| | | | | | (ethyl(2-(methylamino)-2- |
| | | | | | oxoethyl)carbamoyl)pyridin-2-yl)-1- |
| | | | | | isopropyl-4,5,6,7-tetrahydro-1H- |
| | | | | | pyrazolo[4,3-c]pyridine-3-carboxamide |
| 214 | 0.502 | 3 | ND | ND | N-(4-(3,3-dimethylureido)benzyl)-1- |
| | | | | | isopropyl-5-(5-(5-oxo-1,4-diazepane-1- |
| | | | | | carbonyl)pyridin-2-yl)-4,5,6,7-tetrahydro- |
| | | | | | 1H-pyrazolo[4,3-c]pyridine-3-carboxamide |
| 215 | 0.276 | 3 | ND | ND | N-(4-(3,3-dimethylureido)benzyl)-5-(5-(3- |
| | | | | | hydroxyazetidine-1-carbonyl)pyridin-2-yl)-1- |
| | | | | | isopropyl-4,5,6,7-tetrahydro-1H- |
| | | | | | pyrazolo[4,3-c]pyridine-3-carboxamide |
| 216 | 0.546 | 3 | ND | ND | (R)-N-(4-(3,3-dimethylureido)benzyl)-1- |
| | | | | | isopropyl-5-(5-(3-methoxypyrrolidine-1- |
| | | | | | carbonyl)pyridin-2-yl)-4,5,6,7-tetrahydro- |
| | | | | | 1H-pyrazolo[4,3-c]pyridine-3-carboxamide |
| 217 | 0.272 | 3 | ND | ND | 5-(5-(2,2-dimethyl-3-oxopiperazine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 218 | 2.380 | 3 | ND | ND | (R)-5-(5-(2-carbamoylpyrrolidine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 219 | 1.314 | 3 | ND | ND | rac-(R)-N-(4-(3,3-dimethylureido)benzyl)-5- |
| | | | | | (5-(3-hydroxy-3-methylpyrrolidine-1- |
| | | | | | carbonyl)pyridin-2-yl)-1-isopropyl-4,5,6,7- |
| | | | | | tetrahydro-1H-pyrazolo[4,3-c]pyridine-3- |
| | | | | | carboxamide |
| 220 | 0.336 | 3 | ND | ND | rac-(R)-N-(4-(3,3-dimethylureido)benzyl)-5- |
| | | | | | (5-(3-hydroxypiperidine-1-carbonyl)pyridin- |
| | | | | | 2-yl)-1-isopropyl-4,5,6,7-tetrahydro-1H- |
| | | | | | pyrazolo[4,3-c]pyridine-3-carboxamide |
| 221 | 0.227 | 3 | ND | ND | N-(4-(3,3-dimethylureido)benzyl)-1- |
| | | | | | isopropyl-5-(5-(3-oxo-1,4-diazepane-1- |
| | | | | | carbonyl)pyridin-2-yl)-4,5,6,7-tetrahydro- |
| | | | | | 1H-pyrazolo[4,3-c]pyridine-3-carboxamide |
| 222 | 1.478 | 3 | ND | ND | (S)-N-(4-(3,3-dimethylureido)benzyl)-1- |
| | | | | | isopropyl-5-(5-(2- |
| | | | | | (methylcarbamoyl)pyrrolidine-1- |
| | | | | | carbonyl)pyridin-2-yl)-4,5,6,7-tetrahydro- |
| | | | | | 1H-pyrazolo[4,3-c]pyridine-3-carboxamide |
| 223 | 0.201 | 3 | ND | ND | N-(4-(3,3-dimethylureido)benzyl)-1- |
| | | | | | isopropyl-5-(5-(3-oxopiperazine-1- |
| | | | | | carbonyl)pyridin-2-yl)-4,5,6,7-tetrahydro- |
| | | | | | 1H-pyrazolo[4,3-c]pyridine-3-carboxamide |
| 224 | 0.163 | 3 | ND | ND | 5-(5-(4-acetylpiperazine-1-carbonyl)pyridin- |
| | | | | | 2-yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 225 | 0.644 | 3 | ND | ND | (R)-N-(4-(3,3-dimethylureido)benzyl)-1- |
| | | | | | isopropyl-5-(5-(2- |
| | | | | | (methoxymethyl)pyrrolidine-1- |
| | | | | | carbonyl)pyridin-2-yl)-4,5,6,7-tetrahydro- |
| | | | | | 1H-pyrazolo[4,3-c]pyridine-3-carboxamide |
| 226 | 1.932 | 3 | ND | ND | N-(4-(3,3-dimethylureido)benzyl)-1- |
| | | | | | isopropyl-5-(5-(3-methoxy-3- |
| | | | | | methylazetidine-1-carbonyl)pyridin-2-yl)- |
| | | | | | 4,5,6,7-tetrahydro-1H-pyrazolo[4,3- |
| | | | | | c]pyridine-3-carboxamide |
| 227 | >6.116 | 3 | ND | ND | N-(4-(3,3-dimethylureido)benzyl)-1- |
| | | | | | isopropyl-5-(5-((1S,4S)-6-oxo-2,5- |
| | | | | | diazabicyclo[2.2.1]heptane-2- |
| | | | | | carbonyl)pyridin-2-yl)-4,5,6,7-tetrahydro- |
| | | | | | 1H-pyrazolo[4,3-c]pyridine-3-carboxamide |
| 228 | 0.116 | 3 | ND | ND | 5-(5-(2-oxa-6-azaspiro[3.4]octane-6- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 229 | 0.592 | 3 | ND | ND | N-(4-(3,3-dimethylureido)benzyl)-5-(5-((2- |
| | | | | | (ethylamino)-2- |
| | | | | | oxoethyl)(methyl)carbamoyl)pyridin-2-yl)-1- |
| | | | | | isopropyl-4,5,6,7-tetrahydro-1H- |
| | | | | | pyrazolo[4,3-c]pyridine-3-carboxamide |
| 230\hat{ } | 0.014 | 2 | 0.277 | 3 | rel-(5aR,7aS)-5-(5- |
| | | | | | (dimethylcarbamoyl)pyridin-2-yl)-N-(4-(3,3- |
| | | | | | dimethylureido)benzyl)-1-ethyl- |
| | | | | | 4,5,5a,6,7,7a-hexahydro-1H- |
| | | | | | cyclobuta[b]pyrazolo[3,4-d]pyridine-3- |
| | | | | | carboxamide |
| 231\hat{ } | 0.002 | 2 | 0.023 | 2 | rel-(5aR,7aS)-5-(5- |
| | | | | | (dimethylcarbamoyl)pyridin-2-yl)-N-(4-(3,3- |
| | | | | | dimethylureido)benzyl)-1-ethyl- |
| | | | | | 4,5,5a,6,7,7a-hexahydro-1H- |
| | | | | | cyclobuta[b]pyrazolo[3,4-d]pyridine-3- |
| | | | | | carboxamide |
| 232\hat{ } | 0.002 | 2 | 0.005 | 2 | rel-(R or S)-7-(5- |
| | | | | | (dimethylcarbamoyl)pyridin-2-yl)-N-(4-(3,3- |
| | | | | | dimethylureido)benzyl)-3-ethyl-5-methyl- |
| | | | | | 5,6,7,8-tetrahydroimidazo[1,5-a]pyrazine-1- |
| | | | | | carboxamide |
| 233\hat{ } | 0.042 | 2 | 0.078 | 2 | rel-(R or S)-7-(5- |
| | | | | | (dimethylcarbamoyl)pyridin-2-yl)-N-(4-(3,3- |
| | | | | | dimethylureido)benzyl)-3-ethyl-5-methyl- |
| | | | | | 5,6,7,8-tetrahydroimidazo[1,5-a]pyrazine-1- |
| | | | | | carboxamide |
| 234\hat{ } | 0.020 | 2 | 0.080 | 1 | rel-(R or S)-1-(sec-butyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 235\hat{ } | 0.015 | 2 | 0.085 | 1 | rel-(R or S)-1-(sec-butyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 236 | 0.017 | 2 | 0.254 | 1 | ethyl (4-((5-(5-(dimethylcarbamoyl)pyridin- |
| | | | | | 2-yl)-1-isopropyl-4,5,6,7-tetrahydro-1H- |
| | | | | | pyrazolo[4,3-c]pyridine-3- |
| | | | | | carboxamido)methyl)phenyl)carbamate |
| 237\hat{ } | 0.007 | 2 | 0.083 | 1 | rel-(R or S)-1-(1-cyclopropylethyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 238 | 0.002 | 2 | 0.009 | 2 | 5-(5-(dimethylcarbamoyl)pyridin-2-yl)-N-(4- |
| | | | | | (3,3-dimethylureido)benzyl)-1-ethyl-7,7- |
| | | | | | dimethyl-4,5,6,7-tetrahydro-1H- |
| | | | | | pyrazolo[4,3-c]pyridine-3-carboxamide |
| 239 | 0.236 | 3 | ND | ND | N-(4-(3,3-dimethylureido)benzyl)-5-(5- |
| | | | | | ((4aR,7aS)-hexahydro-5H-[1,4]dioxino[2,3- |
| | | | | | c]pyrrole-6-carbonyl)pyridin-2-yl)-1- |
| | | | | | isopropyl-4,5,6,7-tetrahydro-1H- |
| | | | | | pyrazolo[4,3-c]pyridine-3-carboxamide |
| 240 | 3.632 | 2 | ND | ND | 5-(5-(3-cyano-3-methylpyrrolidine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 241 | 0.330 | 3 | ND | ND | N-(4-(3,3-dimethylureido)benzyl)-5-(5- |
| | | | | | ((3aS,6aS)-hexahydro-2H-furo[2,3- |
| | | | | | c]pyrrole-5-carbonyl)pyridin-2-yl)-1- |
| | | | | | isopropyl-4,5,6,7-tetrahydro-1H- |
| | | | | | pyrazolo[4,3-c]pyridine-3-carboxamide |
| 242 | 0.352 | 2 | ND | ND | N-(4-(3,3-dimethylureido)benzyl)-5-(5-(3- |
| | | | | | ethoxyazetidine-1-carbonyl)pyridin-2-yl)-1- |
| | | | | | isopropyl-4,5,6,7-tetrahydro-1H- |
| | | | | | pyrazolo[4,3-c]pyridine-3-carboxamide |
| 243 | 1.652 | 2 | ND | ND | N-(4-(3,3-dimethylureido)benzyl)-5-(5-(6- |
| | | | | | hydroxy-3-azabicyclo[3.1.1]heptane-3- |
| | | | | | carbonyl)pyridin-2-yl)-1-isopropyl-4,5,6,7- |
| | | | | | tetrahydro-1H-pyrazolo[4,3-c]pyridine-3- |
| | | | | | carboxamide |
| 244 | 0.196 | 2 | ND | ND | N-(4-(3,3-dimethylureido)benzyl)-5-(5-(3- |
| | | | | | hydroxy-3-methylazetidine-1- |
| | | | | | carbonyl)pyridin-2-yl)-1-isopropyl-4,5,6,7- |
| | | | | | tetrahydro-1H-pyrazolo[4,3-c]pyridine-3- |
| | | | | | carboxamide |
| 245 | 2.092 | 3 | ND | ND | rac-(R)-N-(4-(3,3-dimethylureido)benzyl)-1- |
| | | | | | isopropyl-5-(5-(2- |
| | | | | | (methylcarbamoyl)pyrrolidine-1- |
| | | | | | carbonyl)pyridin-2-yl)-4,5,6,7-tetrahydro- |
| | | | | | 1H-pyrazolo[4,3-c]pyridine-3-carboxamide |
| 246 | 0.001 | 2 | 0.010 | 2 | rac-(R)-5-(5-(dimethylcarbamoyl)pyridin-2- |
| | | | | | yl)-N-(4-(3,3-dimethylureido)benzyl)-7- |
| | | | | | ethyl-1-isopropyl-4,5,6,7-tetrahydro-1H- |
| | | | | | pyrazolo[4,3-c]pyridine-3-carboxamide |
| 247 | 0.001 | 2 | 0.009 | 2 | 5-(5-(dimethylcarbamoyl)pyridin-2-yl)-N-(4- |
| | | | | | (3,3-dimethylureido)benzyl)-1-isopropyl- |
| | | | | | 7,7-dimethyl-4,5,6,7-tetrahydro-1H- |
| | | | | | pyrazolo[4,3-c]pyridine-3-carboxamide |
| 248 | 0.035 | 2 | 0.131 | 2 | 1-((3,3-difluorocyclobutyl)methyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 249 | 0.006 | 2 | 0.031 | 1 | rac-(R)-5-(5-(dimethylcarbamoyl)pyridin-2- |
| | | | | | yl)-N-(4-(3,3-dimethylureido)benzyl)-6- |
| | | | | | ethyl-1-isopropyl-4,5,6,7-tetrahydro-1H- |
| | | | | | pyrazolo[4,3-c]pyridine-3-carboxamide |
| 250\hat{ } | 0.002 | 2 | 0.028 | 2 | rel-(R or S)-5-(5- |
| | | | | | (dimethylcarbamoyl)pyridin-2-yl)-N-(4-(3,3- |
| | | | | | dimethylureido)benzyl)-1-ethyl-6- |
| | | | | | (trifluoromethyl)-4,5,6,7-tetrahydro-1H- |
| | | | | | pyrazolo[4,3-c]pyridine-3-carboxamide |
| 251\hat{ } | 0.010 | 2 | 0.080 | 2 | rel-(R or S)-5-(5- |
| | | | | | (dimethylcarbamoyl)pyridin-2-yl)-N-(4-(3,3- |
| | | | | | dimethylureido)benzyl)-1-ethyl-6- |
| | | | | | (trifluoromethyl)-4,5,6,7-tetrahydro-1H- |
| | | | | | pyrazolo[4,3-c]pyridine-3-carboxamide |
| 252 | 0.385 | 2 | 1.131 | 2 | 6-(10-(4-(3,3-dimethylureido)benzyl)-11- |
| | | | | | oxo-1,3,4,7,8,9,10,11-octahydro-2H- |
| | | | | | pyrido[4′,3′:3,4]pyrazolo[1,5-a][1,4]diazepin- |
| | | | | | 2-yl)-N,N-dimethylnicotinamide |
| 253 | 0.007 | 2 | 0.126 | 2 | 5-(5-(dimethylcarbamoyl)pyridin-2-yl)-N-(4- |
| | | | | | (3,3-dimethylureido)benzyl)-1-neopentyl- |
| | | | | | 4,5,6,7-tetrahydro-1H-pyrazolo[4,3- |
| | | | | | c]pyridine-3-carboxamide |
| 254 | 0.022 | 2 | 0.180 | 2 | 5-(5-(dimethylcarbamoyl)pyridin-2-yl)-N-(4- |
| | | | | | (3,3-dimethylureido)benzyl)-1-(tetrahydro- |
| | | | | | 2H-pyran-3-yl)-4,5,6,7-tetrahydro-1H- |
| | | | | | pyrazolo[4,3-c]pyridine-3-carboxamide |
| 255 | 0.003 | 2 | 0.048 | 3 | rac-(R)-5-(5-(dimethylcarbamoyl)pyridin-2- |
| | | | | | yl)-N-(4-(3,3-dimethylureido)benzyl)-1- |
| | | | | | isopropyl-6-(trifluoromethyl)-4,5,6,7- |
| | | | | | tetrahydro-1H-pyrazolo[4,3-c]pyridine-3- |
| | | | | | carboxamide |
| 256 | 0.028 | 2 | 0.190 | 2 | 6-(9-(4-(3,3-dimethylureido)benzyl)-10-oxo- |
| | | | | | 3,4,7,8,9,10- |
| | | | | | hexahydropyrido[4′,3′:3,4]pyrazolo[1,5- |
| | | | | | a]pyrazin-2(1H)-yl)-N,N- |
| | | | | | dimethylnicotinamide |
| 257 | 0.015 | 2 | 0.272 | 2 | 1-(cyclobutylmethyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 258\hat{ } | 0.002 | 2 | 0.008 | 3 | (S)-5-(5-(dimethylcarbamoyl)pyridin-2-yl)- |
| | | | | | N-(4-(3,3-dimethylureido)benzyl)-7-methyl- |
| | | | | | 1-(tetrahydro-2H-pyran-4-yl)-4,5,6,7- |
| | | | | | tetrahydro-1H-pyrazolo[4,3-c]pyridine-3- |
| | | | | | carboxamide |
| 259 | 0.002 | 2 | 0.030 | 2 | (S)-N-(4-(3,3-dimethylureido)benzyl)-1,6- |
| | | | | | dimethyl-5-(5-(1-methyl-1H-imidazol-2- |
| | | | | | yl)pyridin-2-yl)-4,5,6,7-tetrahydro-1H- |
| | | | | | pyrazolo[4,3-c]pyridine-3-carboxamide |
| 260 | 0.010 | 2 | 0.064 | 4 | rac-(R)-5-(5-(dimethylcarbamoyl)pyridin-2- |
| | | | | | yl)-N-(4-(3,3-dimethylureido)benzyl)-6- |
| | | | | | ethyl-1-methyl-4,5,6,7-tetrahydro-1H- |
| | | | | | pyrazolo[4,3-c]pyridine-3-carboxamide |
| 261 | 3.573 | 2 | >30.0 | 1 | 5-(5-(dimethylcarbamoyl)pyridin-2-yl)-N-(4- |
| | | | | | (3,3-dimethylureido)benzyl)-1-(tert-pentyl)- |
| | | | | | 4,5,6,7-tetrahydro-1H-pyrazolo[4,3- |
| | | | | | c]pyridine-3-carboxamide |
| 262\hat{ } | 0.001 | 2 | 0.006 | 3 | (S)-5-(5-(dimethylcarbamoyl)pyridin-2-yl)- |
| | | | | | N-(4-(3,3-dimethylureido)benzyl)-7-methyl- |
| | | | | | 1-((RS)-1,1,1-trifluoropropan-2-yl)-4,5,6,7- |
| | | | | | tetrahydro-1H-pyrazolo[4,3-c]pyridine-3- |
| | | | | | carboxamide |
| 263 | 0.009 | 2 | 0.130 | 3 | 1-(1-cyclobutylethyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 264 | 0.007 | 2 | 0.131 | 4 | 1-cyclopentyl-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 265 | 0.005 | 2 | 0.037 | 3 | rac-(R)-5-(5-(dimethylcarbamoyl)pyridin-2- |
| | | | | | yl)-N-(4-(3,3-dimethylureido)benzyl)-7- |
| | | | | | isopropyl-1-methyl-4,5,6,7-tetrahydro-1H- |
| | | | | | pyrazolo[4,3-c]pyridine-3-carboxamide |
| 266 | 0.003 | 2 | 0.045 | 2 | 5′-(5-(dimethylcarbamoyl)pyridin-2-yl)-N-(4- |
| | | | | | (3,3-dimethylureido)benzyl)-1′-isopropyl- |
| | | | | | 1′,4′,5′,6′-tetrahydrospiro[cyclopropane-1,7′- |
| | | | | | pyrazolo[4,3-c]pyridine]-3′-carboxamide |
| 267\hat{ } | 0.050 | 2 | 0.984 | 2 | rel-(R or S)-5-(5- |
| | | | | | (dimethylcarbamoyl)pyridin-2-yl)-N-(4-(3,3- |
| | | | | | dimethylureido)benzyl)-6-ethyl-1-isopropyl- |
| | | | | | 4,5,6,7-tetrahydro-1H-pyrazolo[4,3- |
| | | | | | c]pyridine-3-carboxamide |
| 268\hat{ } | 0.003 | 2 | 0.021 | 2 | rel-(R or S)-5-(5- |
| | | | | | (dimethylcarbamoyl)pyridin-2-yl)-N-(4-(3,3- |
| | | | | | dimethylureido)benzyl)-6-ethyl-1-isopropyl- |
| | | | | | 4,5,6,7-tetrahydro-1H-pyrazolo[4,3- |
| | | | | | c]pyridine-3-carboxamide |
| 269\hat{ } | 0.087 | 2 | 0.834 | 4 | rel-(R or S)-5-(5- |
| | | | | | (dimethylcarbamoyl)pyridin-2-yl)-N-(4-(3,3- |
| | | | | | dimethylureido)benzyl)-6-ethyl-1-methyl- |
| | | | | | 4,5,6,7-tetrahydro-1H-pyrazolo[4,3- |
| | | | | | c]pyridine-3-carboxamide |
| 270\hat{ } | 0.007 | 2 | 0.033 | 4 | rel-(R or S)-5-(5- |
| | | | | | (dimethylcarbamoyl)pyridin-2-yl)-N-(4-(3,3- |
| | | | | | dimethylureido)benzyl)-6-ethyl-1-methyl- |
| | | | | | 4,5,6,7-tetrahydro-1H-pyrazolo[4,3- |
| | | | | | c]pyridine-3-carboxamide |
| 271\hat{ } | 0.011 | 2 | 0.180 | 3 | rel-(R or S)-5-(5- |
| | | | | | (dimethylcarbamoyl)pyridin-2-yl)-N-(4-(3,3- |
| | | | | | dimethylureido)benzyl)-7-isopropyl-1- |
| | | | | | methyl-4,5,6,7-tetrahydro-1H-pyrazolo[4,3- |
| | | | | | c]pyridine-3-carboxamide |
| 272\hat{ } | 0.004 | 2 | 0.030 | 3 | rel-(R or S)-5-(5- |
| | | | | | (dimethylcarbamoyl)pyridin-2-yl)-N-(4-(3,3- |
| | | | | | dimethylureido)benzyl)-7-isopropyl-1- |
| | | | | | methyl-4,5,6,7-tetrahydro-1H-pyrazolo[4,3- |
| | | | | | c]pyridine-3-carboxamide |
| 273\hat{ } | 0.003 | 2 | 0.012 | 3 | (S)-5-(5-(dimethylcarbamoyl)pyrimidin-2- |
| | | | | | yl)-N-(4-(3,3-dimethylureido)benzyl)-1,7- |
| | | | | | dimethyl-4,5,6,7-tetrahydro-1H- |
| | | | | | pyrazolo[4,3-c]pyridine-3-carboxamide |
| 274\hat{ } | 0.695 | 2 | 4.773 | 3 | rel-(4R,8S)-10-(5- |
| | | | | | (dimethylcarbamoyl)pyridin-2-yl)-N-(4-(3,3- |
| | | | | | dimethylureido)benzyl)-1-methyl- |
| | | | | | 4,5,6,7,8,9-hexahydro-1H-4,8- |
| | | | | | epiminocycloocta[c]pyrazole-3- |
| | | | | | carboxamide |
| 275\hat{ } | 0.007 | 2 | 0.030 | 5 | rel-(4R,8S)-10-(5- |
| | | | | | (dimethylcarbamoyl)pyridin-2-yl)-N-(4-(3,3- |
| | | | | | dimethylureido)benzyl)-1-methyl- |
| | | | | | 4,5,6,7,8,9-hexahydro-1H-4,8- |
| | | | | | epiminocycloocta[c]pyrazole-3- |
| | | | | | carboxamide |
| 276\hat{ } | 0.002 | 2 | 0.028 | 2 | rel-(R or S)-5-(5- |
| | | | | | (dimethylcarbamoyl)pyridin-2-yl)-N-(4-(3,3- |
| | | | | | dimethylureido)benzyl)-1-isopropyl-6- |
| | | | | | (trifluoromethyl)-4,5,6,7-tetrahydro-1H- |
| | | | | | pyrazolo[4,3-c]pyridine-3-carboxamide |
| 277\hat{ } | 0.010 | 2 | 0.109 | 2 | rel-(R or S)-5-(5- |
| | | | | | (dimethylcarbamoyl)pyridin-2-yl)-N-(4-(3,3- |
| | | | | | dimethylureido)benzyl)-1-isopropyl-6- |
| | | | | | (trifluoromethyl)-4,5,6,7-tetrahydro-1H- |
| | | | | | pyrazolo[4,3-c]pyridine-3-carboxamide |
| 278 | 0.005 | 2 | 0.253 | 3 | 1-(3,3-dimethylbutan-2-yl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 279 | 0.009 | 2 | 0.094 | 3 | 5-(5-(dimethylcarbamoyl)pyridin-2-yl)-N-(4- |
| | | | | | (3,3-dimethylureido)benzyl)-1-isobutyl- |
| | | | | | 4,5,6,7-tetrahydro-1H-pyrazolo[4,3- |
| | | | | | c]pyridine-3-carboxamide |
| 280\hat{ } | 0.010 | 2 | 0.134 | 2 | rel-(R or S)-6-(9-(4-(3,3- |
| | | | | | dimethylureido)benzyl)-4-methyl-10-oxo- |
| | | | | | 3,4,7,8,9,10- |
| | | | | | hexahydropyrido[4′,3′:3,4]pyrazolo[1,5- |
| | | | | | alpyrazin-2(1H)-yl)-N,N- |
| | | | | | dimethylnicotinamide |
| 281\hat{ } | 0.017 | 2 | 0.212 | 2 | rel-(R or S)-6-(9-(4-(3,3- |
| | | | | | dimethylureido)benzyl)-4-methyl-10-oxo- |
| | | | | | 3,4,7,8,9,10- |
| | | | | | hexahydropyrido[4′,3′:3,4]pyrazolo[1,5- |
| | | | | | alpyrazin-2(1H)-yl)-N,N- |
| | | | | | dimethylnicotinamide |
| 282 | 0.001 | 2 | 0.009 | 2 | rac-(R)-5-(5-(dimethylcarbamoyl)pyridin-2- |
| | | | | | yl)-N-(4-(3,3-dimethylureido)benzyl)-1- |
| | | | | | ethyl-7-(trifluoromethyl)-4,5,6,7-tetrahydro- |
| | | | | | 1H-pyrazolo[4,3-c]pyridine-3-carboxamide |
| 283 | 0.002 | 5 | 0.011 | 4 | (S)-N-(4-(1H-imidazol-2-yl)benzyl)-5-(5- |
| | | | | | (dimethylcarbamoyl)pyridin-2-yl)-1-ethyl-7- |
| | | | | | methyl-4,5,6,7-tetrahydro-1H-pyrazolo[4,3- |
| | | | | | c]pyridine-3-carboxamide |
| 284 | 0.008 | 3 | 0.040 | 2 | (S)-5-(5-(dimethylcarbamoyl)pyridin-2-yl)-1- |
| | | | | | ethyl-7-methyl-N-(4- |
| | | | | | (methylcarbamoyl)benzyl)-4,5,6,7- |
| | | | | | tetrahydro-1H-pyrazolo[4,3-c]pyridine-3- |
| | | | | | carboxamide |
| 285\hat{ } | 0.001 | 2 | 0.007 | 3 | (S)-5-(5-(dimethylcarbamoyl)pyridin-2-yl)- |
| | | | | | N-(4-(3,3-dimethylureido)benzyl)-7-methyl- |
| | | | | | 1-(1-methylcyclopropyl)-4,5,6,7-tetrahydro- |
| | | | | | 1H-pyrazolo[4,3-c]pyridine-3-carboxamide |
| 286 | 0.663 | 2 | >3.065 | 4 | 5-(5-(2-(dimethylamino)-2-oxoethyl)pyridin- |
| | | | | | 2-yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 287\hat{ } | 0.001 | 2 | 0.013 | 4 | (S)-1-(cyclopropylmethyl)-5-(5- |
| | | | | | (dimethylcarbamoyl)pyridin-2-yl)-N-(4-(3,3- |
| | | | | | dimethylureido)benzyl)-7-methyl-4,5,6,7- |
| | | | | | tetrahydro-1H-pyrazolo[4,3-c]pyridine-3- |
| | | | | | carboxamide |
| 288 | 1.639 | 2 | ND | ND | N-(4-(3,3-dimethylureido)benzyl)-1- |
| | | | | | isopropyl-5-(5-(6-oxo-8-oxa-2,5- |
| | | | | | diazaspiro[3.5]nonane-2-carbonyl)pyridin- |
| | | | | | 2-yl)-4,5,6,7-tetrahydro-1H-pyrazolo[4,3- |
| | | | | | c]pyridine-3-carboxamide |
| 289 | 0.312 | 2 | ND | ND | N-(4-(3,3-dimethylureido)benzyl)-5-(5- |
| | | | | | ((3aR,6aS)-hexahydro-1H-furo[3,4- |
| | | | | | c]pyrrole-5-carbonyl)pyridin-2-yl)-1- |
| | | | | | isopropyl-4,5,6,7-tetrahydro-1H- |
| | | | | | pyrazolo[4,3-c]pyridine-3-carboxamide |
| 290 | 1.408 | 2 | ND | ND | rac-N-(4-(3,3-dimethylureido)benzyl)-1- |
| | | | | | isopropyl-5-(5-((3aR,6aS)-2-oxohexahydro- |
| | | | | | 2H-pyrrolo[3,4-d]oxazole-5- |
| | | | | | carbonyl)pyridin-2-yl)-4,5,6,7-tetrahydro- |
| | | | | | 1H-pyrazolo[4,3-c]pyridine-3-carboxamide |
| 291 | 0.793 | 2 | ND | ND | (S)-5-(5-(3-cyanopyrrolidine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 292 | 0.990 | 2 | ND | ND | 5-(5-(3-acetamidoazetidine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 293 | 0.100 | 2 | ND | ND | (R)-5-(5-(3,4-dimethyl-5-oxopiperazine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 294 | 0.961 | 2 | ND | ND | 5-(5-(3,3-dimethyl-5-oxopiperazine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 295 | 0.310 | 2 | ND | ND | N-(4-(3,3-dimethylureido)benzyl)-5-(5-(4- |
| | | | | | hydroxypiperidine-1-carbonyl)pyridin-2-yl)- |
| | | | | | 1-isopropyl-4,5,6,7-tetrahydro-1H- |
| | | | | | pyrazolo[4,3-c]pyridine-3-carboxamide |
| 296 | 0.523 | 2 | ND | ND | 5-(5-((3aR,6aS)-5- |
| | | | | | acetyloctahydropyrrolo[3,4-c]pyrrole-2- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 297 | 0.225 | 2 | ND | ND | (R)-5-(5-(3-cyanopyrrolidine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 298 | 2.545 | 2 | ND | ND | N-(4-(3,3-dimethylureido)benzyl)-1- |
| | | | | | isopropyl-5-(5-(6-oxo-2,5- |
| | | | | | diazaspiro[3.4]octane-2-carbonyl)pyridin-2- |
| | | | | | yl)-4,5,6,7-tetrahydro-1H-pyrazolo[4,3- |
| | | | | | c]pyridine-3-carboxamide |
| 299 | 0.241 | 2 | ND | ND | 5-(5-((3S,5R)-4-acetyl-3,5- |
| | | | | | dimethylpiperazine-1-carbonyl)pyridin-2-yl)- |
| | | | | | N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 300 | 1.416 | 2 | ND | ND | N-(4-(3,3-dimethylureido)benzyl)-1- |
| | | | | | isopropyl-5-(5-(6-methyl-7-oxo-2,6- |
| | | | | | diazaspiro[3.4]octane-2-carbonyl)pyridin-2- |
| | | | | | yl)-4,5,6,7-tetrahydro-1H-pyrazolo[4,3- |
| | | | | | c]pyridine-3-carboxamide |
| 301 | 0.001 | 4 | 0.008 | 4 | (S)-N-(4-(1H-imidazol-2-yl)benzyl)-5-(5- |
| | | | | | (dimethylcarbamoyl)pyrimidin-2-yl)-1-ethyl- |
| | | | | | 7-methyl-4,5,6,7-tetrahydro-1H- |
| | | | | | pyrazolo[4,3-c]pyridine-3-carboxamide |
| 302 | 0.006 | 3 | 0.009 | 2 | (S)-5-(5-(dimethylcarbamoyl)pyridin-2-yl)- |
| | | | | | N-((5-(3,3-dimethylureido)pyridin-2- |
| | | | | | yl)methyl)-1-ethyl-7-methyl-4,5,6,7- |
| | | | | | tetrahydro-1H-pyrazolo[4,3-c]pyridine-3- |
| | | | | | carboxamide |
| 303\hat{ } | 0.022 | 2 | 0.334 | 3 | rel-(R or S)-5-(5- |
| | | | | | (dimethylcarbamoyl)pyridin-2-yl)-N-(4-(3,3- |
| | | | | | dimethylureido)benzyl)-1-ethyl-7- |
| | | | | | (trifluoromethyl)-4,5,6,7-tetrahydro-1H- |
| | | | | | pyrazolo[4,3-c]pyridine-3-carboxamide |
| 304\hat{ } | 0.0003 | 2 | 0.004 | 5 | rel-(R or S)-5-(5- |
| | | | | | (dimethylcarbamoyl)pyridin-2-yl)-N-(4-(3,3- |
| | | | | | dimethylureido)benzyl)-1-ethyl-7- |
| | | | | | (trifluoromethyl)-4,5,6,7-tetrahydro-1H- |
| | | | | | pyrazolo[4,3-c]pyridine-3-carboxamide |
| 305 | 0.005 | 2 | 0.007 | 3 | (S)-5-(5-(dimethylcarbamoyl)pyrimidin-2- |
| | | | | | yl)-N-((5-(3,3-dimethylureido)pyridin-2- |
| | | | | | yl)methyl)-1-ethyl-7-methyl-4,5,6,7- |
| | | | | | tetrahydro-1H-pyrazolo[4,3-c]pyridine-3- |
| | | | | | carboxamide |
| 306 | 0.007 | 2 | 0.033 | 2 | (S)-5-(5-(dimethylcarbamoyl)pyrimidin-2- |
| | | | | | yl)-1-ethyl-7-methyl-N-(4- |
| | | | | | (methylcarbamoyl)benzyl)-4,5,6,7- |
| | | | | | tetrahydro-1H-pyrazolo[4,3-c]pyridine-3- |
| | | | | | carboxamide |
| 307\hat{ } | 0.0005 | 2 | 0.007 | 2 | (S)-5-(5-(dimethylcarbamoyl)pyridin-2-yl)- |
| | | | | | N-(4-(3,3-dimethylureido)benzyl)-7-methyl- |
| | | | | | 1-(2,2,2-trifluoroethyl)-4,5,6,7-tetrahydro- |
| | | | | | 1H-pyrazolo[4,3-c]pyridine-3-carboxamide |
| 308 | 0.287 | 2 | 0.718 | 2 | 1-(2-cyanoethyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 309 | 1.713 | 2 | 4.098 | 2 | 1-(3-amino-3-oxopropyl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 310\hat{ } | 0.008 | 2 | 0.115 | 3 | rel-(R or S)-5-(5- |
| | | | | | (dimethylcarbamoyl)pyridin-2-yl)-N-(4-(3,3- |
| | | | | | dimethylureido)benzyl)-7-ethyl-1-isopropyl- |
| | | | | | 4,5,6,7-tetrahydro-1H-pyrazolo[4,3- |
| | | | | | c]pyridine-3-carboxamide |
| 311\hat{ } | 0.001 | 2 | 0.011 | 4 | rel-(R or S)-5-(5- |
| | | | | | (dimethylcarbamoyl)pyridin-2-yl)-N-(4-(3,3- |
| | | | | | dimethylureido)benzyl)-7-ethyl-1-isopropyl- |
| | | | | | 4,5,6,7-tetrahydro-1H-pyrazolo[4,3- |
| | | | | | c]pyridine-3-carboxamide |
| 312\hat{ } | 0.010 | 2 | 0.163 | 3 | rel-(R or S)-5-(5- |
| | | | | | (dimethylcarbamoyl)pyridin-2-yl)-N-(4-(3,3- |
| | | | | | dimethylureido)benzyl)-1,7-diethyl-4,5,6,7- |
| | | | | | tetrahydro-1H-pyrazolo[4,3-c]pyridine-3- |
| | | | | | carboxamide |
| 313\hat{ } | 0.001 | 2 | 0.015 | 3 | rel-(R or S)-5-(5- |
| | | | | | (dimethylcarbamoyl)pyridin-2-yl)-N-(4-(3,3- |
| | | | | | dimethylureido)benzyl)-1,7-diethyl-4,5,6,7- |
| | | | | | tetrahydro-1H-pyrazolo[4,3-c]pyridine-3- |
| | | | | | carboxamide |
| 314 | 1.073 | 2 | 6.852 | 3 | rac-(6R,7S)-5-(5- |
| | | | | | (dimethylcarbamoyl)pyridin-2-yl)-N-(4-(3,3- |
| | | | | | dimethylureido)benzyl)-2-ethyl-6,7- |
| | | | | | dimethyl-4,5,6,7-tetrahydro-2H- |
| | | | | | pyrazolo[4,3-c]pyridine-3-carboxamide |
| 315 | 0.002 | 2 | ND | ND | (S)-N-(4-(3,3-bis(methyl-d3)ureido)benzyl)- |
| | | | | | 5-(4-(dimethylcarbamoyl)phenyl)-1- |
| | | | | | isopropyl-7-methyl-4,5,6,7-tetrahydro-1H- |
| | | | | | pyrazolo[4,3-c]pyridine-3-carboxamide |
| 316\hat{ } | 3.769 | 1 | ND | ND | rel-(6S,7S)-5-(5- |
| | | | | | (dimethylcarbamoyl)pyridin-2-yl)-N-(4-(3,3- |
| | | | | | dimethylureido)benzyl)-2-ethyl-6,7- |
| | | | | | dimethyl-4,5,6,7-tetrahydro-2H- |
| | | | | | pyrazolo[4,3-c]pyridine-3-carboxamide |
| 317\hat{ } | 0.355 | 2 | ND | ND | rel-(6R,7R)-5-(5- |
| | | | | | (dimethylcarbamoyl)pyridin-2-yl)-N-(4-(3,3- |
| | | | | | dimethylureido)benzyl)-2-ethyl-6,7- |
| | | | | | dimethyl-4,5,6,7-tetrahydro-2H- |
| | | | | | pyrazolo[4,3-c]pyridine-3-carboxamide |
| 318\hat{ } | 0.0004 | 2 | 0.004 | 3 | (S)-1-((R*)-sec-butyl)-5-(5- |
| | | | | | (dimethylcarbamoyl)pyridin-2-yl)-N-(4-(3,3- |
| | | | | | dimethylureido)benzyl)-7-methyl-4,5,6,7- |
| | | | | | tetrahydro-1H-pyrazolo[4,3-c]pyridine-3- |
| | | | | | carboxamide |
| 319\hat{ } | 0.001 | 2 | 0.013 | 2 | (S)-1-((R*)-sec-butyl)-5-(5- |
| | | | | | (dimethylcarbamoyl)pyridin-2-yl)-N-(4-(3,3- |
| | | | | | dimethylureido)benzyl)-7-methyl-4,5,6,7- |
| | | | | | tetrahydro-1H-pyrazolo[4,3-c]pyridine-3- |
| | | | | | carboxamide |
| 320 | 0.005 | 2 | 0.135 | 2 | 5-(5-(dimethylcarbamoyl)pyridin-2-yl)-N-(4- |
| | | | | | (3,3-dimethylureido)benzyl)-1-(1- |
| | | | | | (trifluoromethyl)cyclopropyl)-4,5,6,7- |
| | | | | | tetrahydro-1H-pyrazolo[4,3-c]pyridine-3- |
| | | | | | carboxamide |
| 321 | 0.003 | 2 | 0.022 | 3 | rac-(R)-5-(5-(dimethylcarbamoyl)pyridin-2- |
| | | | | | yl)-N-(4-(3,3-dimethylureido)benzyl)-7- |
| | | | | | methyl-1-(oxetan-3-yl)-4,5,6,7-tetrahydro- |
| | | | | | 1H-pyrazolo[4,3-c]pyridine-3-carboxamide |
| 322 | 0.077 | 2 | 1.351 | 3 | rac-(R)-N-(4-(3,3-dimethylureido)benzyl)-1- |
| | | | | | isopropyl-5-(5-(tetrahydrofuran-2-yl)pyridin- |
| | | | | | 2-yl)-4,5,6,7-tetrahydro-1H-pyrazolo[4,3- |
| | | | | | c]pyridine-3-carboxamide |
| 323 | 0.005 | 2 | 0.026 | 3 | (S)-5-(5-(dimethylcarbamoyl)pyridin-2-yl)- |
| | | | | | N-((6-(3,3-dimethylureido)pyridin-3- |
| | | | | | yl)methyl)-1-ethyl-7-methyl-4,5,6,7- |
| | | | | | tetrahydro-1H-pyrazolo[4,3-c]pyridine-3- |
| | | | | | carboxamide |
| 324 | 0.005 | 2 | 0.018 | 3 | (S)-5-(5-(dimethylcarbamoyl)pyrimidin-2- |
| | | | | | yl)-N-((6-(3,3-dimethylureido)pyridin-3- |
| | | | | | yl)methyl)-1-ethyl-7-methyl-4,5,6,7- |
| | | | | | tetrahydro-1H-pyrazolo[4,3-c]pyridine-3- |
| | | | | | carboxamide |
| 325 | 0.710 | 2 | ND | ND | (R)-N-(4-(3,3-dimethylureido)benzyl)-1- |
| | | | | | isopropyl-5-(5-(3-methyl-5-oxopiperazine-1- |
| | | | | | carbonyl)pyridin-2-yl)-4,5,6,7-tetrahydro- |
| | | | | | 1H-pyrazolo[4,3-c]pyridine-3-carboxamide |
| 326 | 1.285 | 2 | ND | ND | 5-(5-(1-oxa-6-azaspiro[3.3]heptane-6- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 327 | 3.204 | 1 | ND | ND | N-(4-(3,3-dimethylureido)benzyl)-1- |
| | | | | | isopropyl-5-(5-(7-oxo-2,6- |
| | | | | | diazaspiro[3.4]octane-2-carbonyl)pyridin-2- |
| | | | | | yl)-4,5,6,7-tetrahydro-1H-pyrazolo[4,3- |
| | | | | | c]pyridine-3-carboxamide |
| 328 | 0.266 | 2 | ND | ND | 5-(5-(2,2-dimethyl-5-oxopiperazine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 329 | 1.050 | 2 | ND | ND | N-(4-(3,3-dimethylureido)benzyl)-1- |
| | | | | | isopropyl-5-(5-(2-oxo-1,8- |
| | | | | | diazaspiro[4.5]decane-8-carbonyl)pyridin-2- |
| | | | | | yl)-4,5,6,7-tetrahydro-1H-pyrazolo[4,3- |
| | | | | | c]pyridine-3-carboxamide |
| 330 | 1.980 | 2 | ND | ND | 5-(5-(3-acetamido-3-methylazetidine-1- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 331 | 0.606 | 2 | ND | ND | rac-N-(4-(3,3-dimethylureido)benzyl)-5-(5- |
| | | | | | ((3aR,6aR)-hexahydro-2H-furo[2,3- |
| | | | | | c]pyrrole-5-carbonyl)pyridin-2-yl)-1- |
| | | | | | isopropyl-4,5,6,7-tetrahydro-1H- |
| | | | | | pyrazolo[4,3-c]pyridine-3-carboxamide |
| 332 | 0.160 | 2 | ND | ND | N-(4-(3,3-dimethylureido)benzyl)-5-(5-((2- |
| | | | | | hydroxyethyl)(methyl)carbamoyl)pyridin-2- |
| | | | | | yl)-1-isopropyl-4,5,6,7-tetrahydro-1H- |
| | | | | | pyrazolo[4,3-c]pyridine-3-carboxamide |
| 333 | 2.803 | 2 | ND | ND | rac-5-(5-((3R,4R)-3,4-dimethoxypyrrolidine- |
| | | | | | 1-carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 334 | 0.176 | 2 | ND | ND | rac-N-(4-(3,3-dimethylureido)benzyl)-1- |
| | | | | | isopropyl-5-(5-((3aR,6aS)-3-methyl-2- |
| | | | | | oxohexahydro-2H-pyrrolo[3,4-d]oxazole-5- |
| | | | | | carbonyl)pyridin-2-yl)-4,5,6,7-tetrahydro- |
| | | | | | 1H-pyrazolo[4,3-c]pyridine-3-carboxamide |
| 335\hat{ } | 0.001 | 2 | 0.005 | 6 | (S)-N-(4-(1H-imidazol-2-yl)benzyl)-5-(5- |
| | | | | | (dimethylcarbamoyl)pyrimidin-2-yl)-1- |
| | | | | | isopropyl-7-methyl-4,5,6,7-tetrahydro-1H- |
| | | | | | pyrazolo[4,3-c]pyridine-3-carboxamide |
| 336\hat{ } | 0.014 | 2 | 0.041 | 3 | rel-(R or S)-7-(5- |
| | | | | | (dimethylcarbamoyl)pyridin-2-yl)-N-(4-(3,3- |
| | | | | | dimethylureido)benzyl)-5-ethyl-3-methyl- |
| | | | | | 5,6,7,8-tetrahydroimidazo[1,5-a]pyrazine-1- |
| | | | | | carboxamide |
| 337 | 5.367 | 2 | >20.47 | 4 | N-(4-(3,3-dimethylureido)benzyl)-1- |
| | | | | | isopropyl-5-(5-(2-(methylamino)-2- |
| | | | | | oxoethyl)pyridin-2-yl)-4,5,6,7-tetrahydro- |
| | | | | | 1H-pyrazolo[4,3-c]pyridine-3-carboxamide |
| 338 | 3.109 | 2 | >30.0 | 1 | N-(4-(3,3-dimethylureido)benzyl)-1- |
| | | | | | isopropyl-5-(5-(oxetan-3-yl)pyridin-2-yl)- |
| | | | | | 4,5,6,7-tetrahydro-1H-pyrazolo[4,3- |
| | | | | | c]pyridine-3-carboxamide |
| 339 | 6.546 | 2 | >23.12 | 4 | rac-(R)-N-(4-(3,3-dimethylureido)benzyl)-1- |
| | | | | | isopropyl-5-(5-(tetrahydro-2H-pyran-2- |
| | | | | | yl)pyridin-2-yl)-4,5,6,7-tetrahydro-1H- |
| | | | | | pyrazolo[4,3-c]pyridine-3-carboxamide |
| 340 | 0.276 | 2 | 2.343 | 3 | (S)-N-(4-(3,3-dimethylureido)benzyl)-1- |
| | | | | | isopropyl-7-methyl-5-(5-(1-methyl-1H- |
| | | | | | imidazol-2-yl)pyrimidin-2-yl)-4,5,6,7- |
| | | | | | tetrahydro-1H-pyrazolo[4,3-c]pyridine-3- |
| | | | | | carboxamide |
| 341 | 0.004 | 2 | 0.028 | 3 | (S)-5-(5-(dimethylcarbamoyl)pyridin-2-yl)-1- |
| | | | | | isopropyl-7-methyl-N-(4- |
| | | | | | (methylcarbamoyl)benzyl)-4,5,6,7- |
| | | | | | tetrahydro-1H-pyrazolo[4,3-c]pyridine-3- |
| | | | | | carboxamide |
| 342 | 0.003 | 2 | 0.015 | 3 | (S)-N-(4-carbamoylbenzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 343 | 0.001 | 2 | 0.009 | 4 | (S)-N-(4-(1H-imidazol-2-yl)benzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 344 | 0.001 | 2 | 0.008 | 4 | (S)-5-(5-(dimethylcarbamoyl)pyridin-2-yl)- |
| | | | | | N-(4-(3,3-dimethylureido)benzyl)-1-((RS)-1- |
| | | | | | methoxypropan-2-yl)-7-methyl-4,5,6,7- |
| | | | | | tetrahydro-1H-pyrazolo[4,3-c]pyridine-3- |
| | | | | | carboxamide |
| 345 | >2.634 | 2 | >30.0 | 1 | rac-(R)-N-(4-(3,3-dimethylureido)benzyl)-1- |
| | | | | | isopropyl-5-(5-(tetrahydrofuran-3-yl)pyridin- |
| | | | | | 2-yl)-4,5,6,7-tetrahydro-1H-pyrazolo[4,3- |
| | | | | | c]pyridine-3-carboxamide |
| 346 | 0.002 | 2 | 0.013 | 1 | (S)-5-(5-(6,7-dihydro-5H-pyrrolo[3,4- |
| | | | | | b]pyridine-6-carbonyl)pyrimidin-2-yl)-N-(4- |
| | | | | | (3,3-dimethylureido)benzyl)-1-isopropyl-7- |
| | | | | | methyl-4,5,6,7-tetrahydro-1H-pyrazolo[4,3- |
| | | | | | c]pyridine-3-carboxamide |
| 347\hat{ } | 0.083 | 2 | 0.127 | 1 | rel-(R or S)-1-(1-cyanopropan-2-yl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 348\hat{ } | 0.043 | 2 | 0.036 | 1 | rel-(R or S)-1-(1-cyanopropan-2-yl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 349 | 0.002 | 2 | 0.011 | 3 | (S)-5-(5-(dimethylcarbamoyl)pyridin-2-yl)- |
| | | | | | N-((6-(3,3-dimethylureido)pyridin-3- |
| | | | | | yl)methyl)-1-isopropyl-7-methyl-4,5,6,7- |
| | | | | | tetrahydro-1H-pyrazolo[4,3-c]pyridine-3- |
| | | | | | carboxamide |
| 350 | 0.002 | 2 | 0.006 | 4 | (S)-5-(5-(dimethylcarbamoyl)pyridin-2-yl)- |
| | | | | | N-((5-(3,3-dimethylureido)pyridin-2- |
| | | | | | yl)methyl)-1-isopropyl-7-methyl-4,5,6,7- |
| | | | | | tetrahydro-1H-pyrazolo[4,3-c]pyridine-3- |
| | | | | | carboxamide |
| 351 | 0.073 | 2 | 0.129 | 2 | rac-(R)-5-(5-(dimethylcarbamoyl)pyridin-2- |
| | | | | | yl)-N-(4-(3,3-dimethylureido)benzyl)-1-(1- |
| | | | | | hydroxypropan-2-yl)-4,5,6,7-tetrahydro-1H- |
| | | | | | pyrazolo[4,3-c]pyridine-3-carboxamide |
| 352\hat{ } | 0.0003 | 2 | 0.003 | 5 | (6S,7S)-5-(5-(dimethylcarbamoyl)pyridin-2- |
| | | | | | yl)-N-(4-(3,3-dimethylureido)benzyl)-1- |
| | | | | | ethyl-6,7-dimethyl-4,5,6,7-tetrahydro-1H- |
| | | | | | pyrazolo[4,3-c]pyridine-3-carboxamide |
| 353\hat{ } | 0.004 | 2 | 0.047 | 3 | (6R,7R)-5-(5-(dimethylcarbamoyl)pyridin-2- |
| | | | | | yl)-N-(4-(3,3-dimethylureido)benzyl)-1- |
| | | | | | ethyl-6,7-dimethyl-4,5,6,7-tetrahydro-1H- |
| | | | | | pyrazolo[4,3-c]pyridine-3-carboxamide |
| 354\hat{ } | 0.004 | 2 | 0.024 | 3 | (6R,7R)-5-(5-(dimethylcarbamoyl)pyridin-2- |
| | | | | | yl)-N-(4-(3,3-dimethylureido)benzyl)-1- |
| | | | | | isopropyl-6,7-dimethyl-4,5,6,7-tetrahydro- |
| | | | | | 1H-pyrazolo[4,3-c]pyridine-3-carboxamide |
| 355\hat{ } | 0.0002 | 2 | 0.003 | 5 | (6S,7S)-5-(5-(dimethylcarbamoyl)pyridin-2- |
| | | | | | yl)-N-(4-(3,3-dimethylureido)benzyl)-1- |
| | | | | | isopropyl-6,7-dimethyl-4,5,6,7-tetrahydro- |
| | | | | | 1H-pyrazolo[4,3-c]pyridine-3-carboxamide |
| 356\hat{ } | 0.246 | 2 | 3.311 | 3 | rel-(R or S)-5-(5- |
| | | | | | (dimethylcarbamoyl)pyridin-2-yl)-1- |
| | | | | | isopropyl-N-(4-(methylcarbamoyl)benzyl)-7- |
| | | | | | (trifluoromethyl)-4,5,6,7-tetrahydro-1H- |
| | | | | | pyrazolo[4,3-c]pyridine-3-carboxamide |
| 357\hat{ } | 0.001 | 2 | 0.020 | 3 | rel-(R or S)-5-(5- |
| | | | | | (dimethylcarbamoyl)pyridin-2-yl)-1- |
| | | | | | isopropyl-N-(4-(methylcarbamoyl)benzyl)-7- |
| | | | | | (trifluoromethyl)-4,5,6,7-tetrahydro-1H- |
| | | | | | pyrazolo[4,3-c]pyridine-3-carboxamide |
| 358 | 0.051 | 2 | 0.112 | 2 | rac-(R)-1-(1-cyanopropan-2-yl)-5-(5- |
| | | | | | (dimethylcarbamoyl)pyridin-2-yl)-N-(4-(3,3- |
| | | | | | dimethylureido)benzyl)-4,5,6,7-tetrahydro- |
| | | | | | 1H-pyrazolo[4,3-c]pyridine-3-carboxamide |
| 359\hat{ } | 0.358 | 2 | 0.869 | 2 | rel-(R or S)-5-(5- |
| | | | | | (dimethylcarbamoyl)pyridin-2-yl)-N-(4-(3,3- |
| | | | | | dimethylureido)benzyl)-1-(1- |
| | | | | | hydroxypropan-2-yl)-4,5,6,7-tetrahydro-1H- |
| | | | | | pyrazolo[4,3-c]pyridine-3-carboxamide |
| 360\hat{ } | 0.074 | 2 | 0.091 | 2 | rel-(R or S)-5-(5- |
| | | | | | (dimethylcarbamoyl)pyridin-2-yl)-N-(4-(3,3- |
| | | | | | dimethylureido)benzyl)-1-(1- |
| | | | | | hydroxypropan-2-yl)-4,5,6,7-tetrahydro-1H- |
| | | | | | pyrazolo[4,3-c]pyridine-3-carboxamide |
| 361\hat{ } | 0.158 | 2 | >4.082 | 4 | rel-(R or S)-N-(4-(3,3- |
| | | | | | dimethylureido)benzyl)-1-isopropyl-5-(5- |
| | | | | | (tetrahydrofuran-2-yl)pyridin-2-yl)-4,5,6,7- |
| | | | | | tetrahydro-1H-pyrazolo[4,3-c]pyridine-3- |
| | | | | | carboxamide |
| 362\hat{ } | 0.838 | 2 | >3.489 | 4 | rel-(R or S)-N-(4-(3,3- |
| | | | | | dimethylureido)benzyl)-1-isopropyl-5-(5- |
| | | | | | (tetrahydrofuran-2-yl)pyridin-2-yl)-4,5,6,7- |
| | | | | | tetrahydro-1H-pyrazolo[4,3-c]pyridine-3- |
| | | | | | carboxamide |
| 363 | 0.007 | 3 | ND | ND | N-(4-(3,3-dimethylureido)benzyl)-1- |
| | | | | | isopropyl-5-(5-(1-methyl-1,4,5,6- |
| | | | | | tetrahydropyrrolo[3,4-c]pyrazole-5- |
| | | | | | carbonyl)pyridin-2-yl)-4,5,6,7-tetrahydro- |
| | | | | | 1H-pyrazolo[4,3-c]pyridine-3-carboxamide |
| 364 | 0.020 | 3 | ND | ND | N-(4-(3,3-dimethylureido)benzyl)-1- |
| | | | | | isopropyl-5-(5-(2-methyl-4,5,6,7-tetrahydro- |
| | | | | | 2H-pyrazolo[4,3-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 365 | 0.006 | 3 | ND | ND | N-(4-(3,3-dimethylureido)benzyl)-1- |
| | | | | | isopropyl-5-(5-(2-methyl-4,5,6,7- |
| | | | | | tetrahydrothiazolo[5,4-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 366 | 0.029 | 3 | ND | ND | N-(4-(3,3-dimethylureido)benzyl)-1- |
| | | | | | isopropyl-5-(5-(5,6,7,8- |
| | | | | | tetrahydropyrido[3,4-b]pyrazine-6- |
| | | | | | carbonyl)pyridin-2-yl)-4,5,6,7-tetrahydro- |
| | | | | | 1H-pyrazolo[4,3-c]pyridine-3-carboxamide |
| 367 | 0.072 | 3 | ND | ND | N-(4-(3,3-dimethylureido)benzyl)-1- |
| | | | | | isopropyl-5-(5-(5,6,7,8-tetrahydro- |
| | | | | | [1,2,4]triazolo[1,5-a]pyrazine-7- |
| | | | | | carbonyl)pyridin-2-yl)-4,5,6,7-tetrahydro- |
| | | | | | 1H-pyrazolo[4,3-c]pyridine-3-carboxamide |
| 368 | 0.005 | 3 | ND | ND | 5-(5-(2,4-dimethyl-6,7-dihydro-5H- |
| | | | | | pyrrolo[3,4-b]pyridine-6-carbonyl)pyridin-2- |
| | | | | | yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 369 | 0.038 | 3 | ND | ND | 5-(5-(1,3-dimethyl-4,5,6,7-tetrahydro-1H- |
| | | | | | pyrazolo[4,3-c]pyridine-5-carbonyl)pyridin- |
| | | | | | 2-yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 370 | 0.011 | 3 | ND | ND | 5-(5-(2-cyclopropyl-6,7-dihydro-5H- |
| | | | | | pyrrolo[3,4-d]pyrimidine-6-carbonyl)pyridin- |
| | | | | | 2-yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 371 | 0.050 | 3 | ND | ND | N-(4-(3,3-dimethylureido)benzyl)-1- |
| | | | | | isopropyl-5-(5-(1-methyl-4,5,6,7-tetrahydro- |
| | | | | | 1H-pyrazolo[3,4-c]pyridine-6- |
| | | | | | carbonyl)pyridin-2-yl)-4,5,6,7-tetrahydro- |
| | | | | | 1H-pyrazolo[4,3-c]pyridine-3-carboxamide |
| 372 | 0.059 | 3 | ND | ND | N-(4-(3,3-dimethylureido)benzyl)-1- |
| | | | | | isopropyl-5-(5-(5,6,7,8- |
| | | | | | tetrahydroimidazo[1,2-a]pyrazine-7- |
| | | | | | carbonyl)pyridin-2-yl)-4,5,6,7-tetrahydro- |
| | | | | | 1H-pyrazolo[4,3-c]pyridine-3-carboxamide |
| 373 | 0.540 | 3 | ND | ND | N-(4-(3,3-dimethylureido)benzyl)-1- |
| | | | | | isopropyl-5-(5-(3-methyl-5,6,7,8-tetrahydro- |
| | | | | | [1,2,4]triazolo[4,3-a]pyrazine-7- |
| | | | | | carbonyl)pyridin-2-yl)-4,5,6,7-tetrahydro- |
| | | | | | 1H-pyrazolo[4,3-c]pyridine-3-carboxamide |
| 374 | 0.014 | 3 | ND | ND | N-(4-(3,3-dimethylureido)benzyl)-1- |
| | | | | | isopropyl-5-(5-(4,5,6,7- |
| | | | | | tetrahydrothiazolo[5,4-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 375 | 0.015 | 4 | 1.962 | 1 | N-(4-(3,3-dimethylureido)benzyl)-1- |
| | | | | | isopropyl-5-(5-(8-methoxy-1,2,3,4- |
| | | | | | tetrahydro-2,7-naphthyridine-2- |
| | | | | | carbonyl)pyridin-2-yl)-4,5,6,7-tetrahydro- |
| | | | | | 1H-pyrazolo[4,3-c]pyridine-3-carboxamide |
| 376 | 0.010 | 3 | ND | ND | N-(4-(3,3-dimethylureido)benzyl)-1- |
| | | | | | isopropyl-5-(5-(5,6,7,8-tetrahydro-1,6- |
| | | | | | naphthyridine-6-carbonyl)pyridin-2-yl)- |
| | | | | | 4,5,6,7-tetrahydro-1H-pyrazolo[4,3- |
| | | | | | c]pyridine-3-carboxamide |
| 377 | 0.021 | 3 | ND | ND | N-(4-(3,3-dimethylureido)benzyl)-1- |
| | | | | | isopropyl-5-(5-(2-methyl-4,5,6,7- |
| | | | | | tetrahydropyrazolo[1,5-a]pyrazine-5- |
| | | | | | carbonyl)pyridin-2-yl)-4,5,6,7-tetrahydro- |
| | | | | | 1H-pyrazolo[4,3-c]pyridine-3-carboxamide |
| 378 | 0.015 | 2 | ND | ND | 5-(5-(2,3-dihydro-1H-pyrrolo[3,4-c]pyridine- |
| | | | | | 2-carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 379 | 0.070 | 3 | ND | ND | N-(4-(3,3-dimethylureido)benzyl)-1- |
| | | | | | isopropyl-5-(5-(5,6,7,8- |
| | | | | | tetrahydropyrido[4,3-d]pyrimidine-6- |
| | | | | | carbonyl)pyridin-2-yl)-4,5,6,7-tetrahydro- |
| | | | | | 1H-pyrazolo[4,3-c]pyridine-3-carboxamide |
| 380 | 0.538 | 3 | ND | ND | N-(4-(3,3-dimethylureido)benzyl)-1- |
| | | | | | isopropyl-5-(5-(5,6,7,8-tetrahydro- |
| | | | | | [1,2,4]triazolo[4,3-a]pyrazine-7- |
| | | | | | carbonyl)pyridin-2-yl)-4,5,6,7-tetrahydro- |
| | | | | | 1H-pyrazolo[4,3-c]pyridine-3-carboxamide |
| 381 | 0.033 | 3 | ND | ND | 5-(5-(2,3-dimethyl-4,5,6,7-tetrahydro-2H- |
| | | | | | pyrazolo[4,3-c]pyridine-5-carbonyl)pyridin- |
| | | | | | 2-yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 382 | 0.137 | 3 | ND | ND | N-(4-(3,3-dimethylureido)benzyl)-1- |
| | | | | | isopropyl-5-(5-(3-methyl-5,6,7,8- |
| | | | | | tetrahydroimidazo[1,5-a]pyrazine-7- |
| | | | | | carbonyl)pyridin-2-yl)-4,5,6,7-tetrahydro- |
| | | | | | 1H-pyrazolo[4,3-c]pyridine-3-carboxamide |
| 383 | 0.049 | 3 | ND | ND | N-(4-(3,3-dimethylureido)benzyl)-1- |
| | | | | | isopropyl-5-(5-(4,5,6,7- |
| | | | | | tetrahydroisoxazolo[4,3-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 384 | 0.015 | 4 | 1.073 | 1 | N-(4-(3,3-dimethylureido)benzyl)-1- |
| | | | | | isopropyl-5-(5-(2-methoxy-5,6,7,8- |
| | | | | | tetrahydro-1,6-naphthyridine-6- |
| | | | | | carbonyl)pyridin-2-yl)-4,5,6,7-tetrahydro- |
| | | | | | 1H-pyrazolo[4,3-c]pyridine-3-carboxamide |
| 385 | 0.004 | 2 | ND | ND | N-(4-(3,3-dimethylureido)benzyl)-1- |
| | | | | | isopropyl-5-(5-(2-methyl-2,4,5,6- |
| | | | | | tetrahydropyrrolo[3,4-c]pyrazole-5- |
| | | | | | carbonyl)pyridin-2-yl)-4,5,6,7-tetrahydro- |
| | | | | | 1H-pyrazolo[4,3-c]pyridine-3-carboxamide |
| 386 | 0.056 | 3 | ND | ND | 5-(5-(2-cyano-4,5,6,7- |
| | | | | | tetrahydropyrazolo[1,5-a]pyrazine-5- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 387 | 0.003 | 2 | ND | ND | N-(4-(3,3-dimethylureido)benzyl)-1- |
| | | | | | isopropyl-5-(5-(4-methoxyisoindoline-2- |
| | | | | | carbonyl)pyridin-2-yl)-4,5,6,7-tetrahydro- |
| | | | | | 1H-pyrazolo[4,3-c]pyridine-3-carboxamide |
| 388 | 0.008 | 2 | 0.029 | 2 | (S)-1-ethyl-7-methyl-N-(4- |
| | | | | | (methylcarbamoyl)benzyl)-5-(5-(2- |
| | | | | | oxopyrrolidin-1-yl)pyridin-2-yl)-4,5,6,7- |
| | | | | | tetrahydro-1H-pyrazolo[4,3-c]pyridine-3- |
| | | | | | carboxamide |
| 389 | 0.006 | 2 | 0.017 | 2 | (S)-N-(4-carbamoylbenzyl)-1-ethyl-7- |
| | | | | | methyl-5-(5-(2-oxopyrrolidin-1-yl)pyridin-2- |
| | | | | | yl)-4,5,6,7-tetrahydro-1H-pyrazolo[4,3- |
| | | | | | c]pyridine-3-carboxamide |
| 390 | 0.039 | 2 | 0.104 | 2 | rel-(R or S)-5-(5- |
| | | | | | (dimethylcarbamoyl)pyridin-2-yl)-N-(4-(3,3- |
| | | | | | dimethylureido)benzyl)-1-methyl-7- |
| | | | | | (trifluoromethyl)-4,5,6,7-tetrahydro-1H- |
| | | | | | pyrazolo[4,3-c]pyridine-3-carboxamide |
| 391\hat{ } | 0.740 | 2 | 2.172 | 1 | rel-(R or S)-5-(5- |
| | | | | | (dimethylcarbamoyl)pyridin-2-yl)-N-(4-(3,3- |
| | | | | | dimethylureido)benzyl)-1-methyl-7- |
| | | | | | (trifluoromethyl)-4,5,6,7-tetrahydro-1H- |
| | | | | | pyrazolo[4,3-c]pyridine-3-carboxamide |
| 392\hat{ } | 0.568 | 2 | 1.082 | 1 | rel-(R or S)-5-(5- |
| | | | | | (dimethylcarbamoyl)pyrimidin-2-yl)-N-(4- |
| | | | | | (3,3-dimethylureido)benzyl)-1-methyl-7- |
| | | | | | (trifluoromethyl)-4,5,6,7-tetrahydro-1H- |
| | | | | | pyrazolo[4,3-c]pyridine-3-carboxamide |
| 393\hat{ } | >8.752 | 2 | >30.0 | 1 | rel-(R or S)-N-(4-(3,3- |
| | | | | | dimethylureido)benzyl)-1-isopropyl-5-(5- |
| | | | | | (tetrahydro-2H-pyran-2-yl)pyridin-2-yl)- |
| | | | | | 4,5,6,7-tetrahydro-1H-pyrazolo[4,3- |
| | | | | | c]pyridine-3-carboxamide |
| 394\hat{ } | >7.658 | 2 | >30.0 | 1 | rel-(R or S)-N-(4-(3,3- |
| | | | | | dimethylureido)benzyl)-1-isopropyl-5-(5- |
| | | | | | (tetrahydro-2H-pyran-2-yl)pyridin-2-yl)- |
| | | | | | 4,5,6,7-tetrahydro-1H-pyrazolo[4,3- |
| | | | | | c]pyridine-3-carboxamide |
| 395 | 0.001 | 2 | 0.006 | 2 | (S)-N-(4-carbamoylbenzyl)-5-(5-(6,7- |
| | | | | | dihydro-5H-pyrrolo[3,4-b]pyridine-6- |
| | | | | | carbonyl)pyridin-2-yl)-1-isopropyl-7-methyl- |
| | | | | | 4,5,6,7-tetrahydro-1H-pyrazolo[4,3- |
| | | | | | c]pyridine-3-carboxamide |
| 396 | 0.001 | 2 | 0.015 | 2 | (S)-5-(5-(6,7-dihydro-5H-pyrrolo[3,4- |
| | | | | | b]pyridine-6-carbonyl)pyridin-2-yl)-1- |
| | | | | | isopropyl-7-methyl-N-(4- |
| | | | | | (methylcarbamoyl)benzyl)-4,5,6,7- |
| | | | | | tetrahydro-1H-pyrazolo[4,3-c]pyridine-3- |
| | | | | | carboxamide |
| 397 | 0.001 | 2 | 0.009 | 2 | (S)-5-(5-(dimethylcarbamoyl)pyrimidin-2- |
| | | | | | yl)-1-ethyl-7-methyl-N-(4-(4-methyl-1H- |
| | | | | | imidazol-2-yl)benzyl)-4,5,6,7-tetrahydro-1H- |
| | | | | | pyrazolo[4,3-c]pyridine-3-carboxamide |
| 398 | 0.0003 | 2 | 0.007 | 2 | (S)-5-(4-(dimethylcarbamoyl)phenyl)-1- |
| | | | | | ethyl-7-methyl-N-(4-(4-methylpiperazine-1- |
| | | | | | carboxamido)benzyl)-4,5,6,7-tetrahydro- |
| | | | | | 1H-pyrazolo[4,3-c]pyridine-3-carboxamide |
| 399 | 0.002 | 2 | 0.009 | 2 | rac-(R)-7-(4-(dimethylcarbamoyl)phenyl)-N- |
| | | | | | (4-(3,3-dimethylureido)benzyl)-3-ethyl-5- |
| | | | | | methyl-5,6,7,8-tetrahydroimidazo[1,5- |
| | | | | | a]lpyrazine-1-carboxamide |
| 400 | 1.042 | 2 | 2.574 | 2 | 5′-(5-(dimethylcarbamoyl)pyridin-2-yl)-N-(4- |
| | | | | | (3,3-dimethylureido)benzyl)-1′-methyl- |
| | | | | | 1′,4′,5′,7′-tetrahydrospiro[cyclopropane-1,6′- |
| | | | | | pyrazolo[4,3-c]pyridine]-3′-carboxamide |
| 401 | 0.032 | 3 | ND | ND | 5-(5-(1,3-dimethyl-1,4,5,6- |
| | | | | | tetrahydropyrrolo[3,4-c]pyrazole-5- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 402 | 0.003 | 4 | ND | ND | 5-(5-(5,6-dihydro-4H-pyrrolo[3,4-d]thiazole- |
| | | | | | 5-carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 403 | 0.012 | 4 | ND | ND | N-(4-(3,3-dimethylureido)benzyl)-1- |
| | | | | | isopropyl-5-(5-(4,5,6,7- |
| | | | | | tetrahydrothiazolo[4,5-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 404 | 0.040 | 4 | ND | ND | N-(4-(3,3-dimethylureido)benzyl)-1- |
| | | | | | isopropyl-5-(5-(3-methyl-5,6,7,8- |
| | | | | | tetrahydroimidazo[1,2-a]pyrazine-7- |
| | | | | | carbonyl)pyridin-2-yl)-4,5,6,7-tetrahydro- |
| | | | | | 1H-pyrazolo[4,3-c]pyridine-3-carboxamide |
| 405 | 0.159 | 4 | ND | ND | N-(4-(3,3-dimethylureido)benzyl)-1- |
| | | | | | isopropyl-5-(5-(3-methyl-4,5,6,7-tetrahydro- |
| | | | | | 3H-imidazo[4,5-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 406 | 0.011 | 4 | ND | ND | N-(4-(3,3-dimethylureido)benzyl)-1- |
| | | | | | isopropyl-5-(5-(2-methyl-4,5,6,7- |
| | | | | | tetrahydrothiazolo[4,5-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 407 | 0.010 | 3 | 0.100 | 1 | N-(4-(3,3-dimethylureido)benzyl)-1- |
| | | | | | isopropyl-5-(5-(4-methoxy-5,6,7,8- |
| | | | | | tetrahydro-1,7-naphthyridine-7- |
| | | | | | carbonyl)pyridin-2-yl)-4,5,6,7-tetrahydro- |
| | | | | | 1H-pyrazolo[4,3-c]pyridine-3-carboxamide |
| 408 | 0.039 | 4 | ND | ND | 5-(5-(6,7-dihydro-5H-pyrrolo[3,4- |
| | | | | | d]pyrimidine-6-carbonyl)pyridin-2-yl)-N-(4- |
| | | | | | (3,3-dimethylureido)benzyl)-1-isopropyl- |
| | | | | | 4,5,6,7-tetrahydro-1H-pyrazolo[4,3- |
| | | | | | c]pyridine-3-carboxamide |
| 409 | 0.024 | 3 | ND | ND | 5-(5-(1,2-dimethyl-4,5,6,7-tetrahydro-1H- |
| | | | | | imidazo[4,5-c]pyridine-5-carbonyl)pyridin-2- |
| | | | | | yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 410 | 0.015 | 2 | ND | ND | N-(4-(3,3-dimethylureido)benzyl)-1- |
| | | | | | isopropyl-5-(5-(3-methyl-5,6,7,8-tetrahydro- |
| | | | | | 1,6-naphthyridine-6-carbonyl)pyridin-2-yl)- |
| | | | | | 4,5,6,7-tetrahydro-1H-pyrazolo[4,3- |
| | | | | | c]pyridine-3-carboxamide |
| 411 | 0.008 | 3 | ND | ND | 5-(5-(5-cyanoisoindoline-2- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 412 | 0.396 | 3 | ND | ND | N-(4-(3,3-dimethylureido)benzyl)-5-(5-(7- |
| | | | | | hydroxy-1,2,3,4-tetrahydro-2,6- |
| | | | | | naphthyridine-2-carbonyl)pyridin-2-yl)-1- |
| | | | | | isopropyl-4,5,6,7-tetrahydro-1H- |
| | | | | | pyrazolo[4,3-c]pyridine-3-carboxamide |
| 413 | 0.082 | 3 | ND | ND | N-(4-(3,3-dimethylureido)benzyl)-1- |
| | | | | | isopropyl-5-(5-(2-methyl-5,6,7,8-tetrahydro- |
| | | | | | [1,2,4]triazolo[1,5-a]pyrazine-7- |
| | | | | | carbonyl)pyridin-2-yl)-4,5,6,7-tetrahydro- |
| | | | | | 1H-pyrazolo[4,3-c]pyridine-3-carboxamide |
| 414 | 0.066 | 3 | ND | ND | 5-(5-(2,3-dimethyl-5,6,7,8- |
| | | | | | tetrahydroimidazo[1,2-a]pyrazine-7- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 415 | 0.003 | 2 | ND | ND | 5-(5-(4-cyanoisoindoline-2- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 416 | 0.029 | 3 | ND | ND | N-(4-(3,3-dimethylureido)benzyl)-1- |
| | | | | | isopropyl-5-(5-(1-methyl-4,5,6,7-tetrahydro- |
| | | | | | 1H-pyrazolo[4,3-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 417 | 0.017 | 3 | ND | ND | N-(4-(3,3-dimethylureido)benzyl)-1- |
| | | | | | isopropyl-5-(5-(5,6,7,8-tetrahydro-1,7- |
| | | | | | naphthyridine-7-carbonyl)pyridin-2-yl)- |
| | | | | | 4,5,6,7-tetrahydro-1H-pyrazolo[4,3- |
| | | | | | c]pyridine-3-carboxamide |
| 418 | 0.061 | 2 | ND | ND | 5-(5-(1,3-dimethyl-4,5,6,7-tetrahydro-1H- |
| | | | | | pyrazolo[3,4-c]pyridine-6-carbonyl)pyridin- |
| | | | | | 2-yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 419 | 0.044 | 3 | ND | ND | N-(4-(3,3-dimethylureido)benzyl)-1- |
| | | | | | isopropyl-5-(5-(3-methyl-4,5,6,7- |
| | | | | | tetrahydropyrazolo[1,5-a]pyrazine-5- |
| | | | | | carbonyl)pyridin-2-yl)-4,5,6,7-tetrahydro- |
| | | | | | 1H-pyrazolo[4,3-c]pyridine-3-carboxamide |
| 420 | 0.017 | 4 | 0.125 | 3 | 5-(5-(1-cyclopropyl-4,5,6,7-tetrahydro-1H- |
| | | | | | imidazo[4,5-c]pyridine-5-carbonyl)pyridin-2- |
| | | | | | yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 421 | 0.018 | 3 | ND | ND | N-(4-(3,3-dimethylureido)benzyl)-1- |
| | | | | | isopropyl-5-(5-(1-methyl-4,5,6,7-tetrahydro- |
| | | | | | 1H-imidazo[4,5-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 422 | 0.019 | 3 | ND | ND | 5-(5-(7-cyano-1,2,3,4- |
| | | | | | tetrahydroisoquinoline-2-carbonyl)pyridin-2- |
| | | | | | yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 423 | 0.029 | 3 | ND | ND | N-(4-(3,3-dimethylureido)benzyl)-1- |
| | | | | | isopropyl-5-(5-(2-methyl-4,5,6,7- |
| | | | | | tetrahydrooxazolo[4,5-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 424 | 0.046 | 2 | ND | ND | N-(4-(3,3-dimethylureido)benzyl)-1- |
| | | | | | isopropyl-5-(5-(7-methyl-4,5,6,7- |
| | | | | | tetrahydropyrazolo[1,5-a]pyrazine-5- |
| | | | | | carbonyl)pyridin-2-yl)-4,5,6,7-tetrahydro- |
| | | | | | 1H-pyrazolo[4,3-c]pyridine-3-carboxamide |
| 425 | 0.034 | 2 | ND | ND | N-(4-(3,3-dimethylureido)benzyl)-1- |
| | | | | | isopropyl-5-(5-(4,5,6,7- |
| | | | | | tetrahydropyrazolo[1,5-a]pyrazine-5- |
| | | | | | carbonyl)pyridin-2-yl)-4,5,6,7-tetrahydro- |
| | | | | | 1H-pyrazolo[4,3-c]pyridine-3-carboxamide |
| 426 | 0.013 | 2 | ND | ND | (R)-N-(4-(3,3-dimethylureido)benzyl)-1- |
| | | | | | isopropyl-5-(5-(1-methylisoindoline-2- |
| | | | | | carbonyl)pyridin-2-yl)-4,5,6,7-tetrahydro- |
| | | | | | 1H-pyrazolo[4,3-c]pyridine-3-carboxamide |
| 427 | 0.282 | 2 | ND | ND | N-(4-(3,3-dimethylureido)benzyl)-1- |
| | | | | | isopropyl-5-(5-(4,5,6,7-tetrahydro- |
| | | | | | [1,2,3]triazolo[1,5-a]pyrazine-5- |
| | | | | | carbonyl)pyridin-2-yl)-4,5,6,7-tetrahydro- |
| | | | | | 1H-pyrazolo[4,3-c]pyridine-3-carboxamide |
| 428\hat{ } | 0.017 | 2 | 0.140 | 2 | rel-(R or S)-7-(4- |
| | | | | | (dimethylcarbamoyl)phenyl)-N-(4-(3,3- |
| | | | | | dimethylureido)benzyl)-3-ethyl-5-methyl- |
| | | | | | 5,6,7,8-tetrahydroimidazo[1,5-a]pyrazine-1- |
| | | | | | carboxamide |
| 429\hat{ } | 0.001 | 2 | 0.006 | 2 | rel-(R or S)-7-(4- |
| | | | | | (dimethylcarbamoyl)phenyl)-N-(4-(3,3- |
| | | | | | dimethylureido)benzyl)-3-ethyl-5-methyl- |
| | | | | | 5,6,7,8-tetrahydroimidazo[1,5-a]pyrazine-1- |
| | | | | | carboxamide |
| 430 | 0.333 | 2 | 1.414 | 2 | 5-(5-(2-amino-2-oxoethyl)pyridin-2-yl)-N-(4- |
| | | | | | (3,3-dimethylureido)benzyl)-1-isopropyl- |
| | | | | | 4,5,6,7-tetrahydro-1H-pyrazolo[4,3- |
| | | | | | c]pyridine-3-carboxamide |
| 431 | 0.012 | 3 | 0.089 | 3 | 5-(5-(4-(dimethylcarbamoyl)isoindoline-2- |
| | | | | | carbonyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 432 | 0.186 | 2 | ND | ND | N-(4-(3,3-dimethylureido)benzyl)-1- |
| | | | | | isopropyl-5-(5-(4,5,6,7- |
| | | | | | tetrahydroisoxazolo[4,5-c]pyridine-5- |
| | | | | | carbonyl)pyridin-2-yl)-4,5,6,7-tetrahydro- |
| | | | | | 1H-pyrazolo[4,3-c]pyridine-3-carboxamide |
| 433 | 0.276 | 2 | ND | ND | N-(4-(3,3-dimethylureido)benzyl)-1- |
| | | | | | isopropyl-5-(5-(5,6,7,8- |
| | | | | | tetrahydropyrido[3,4-d]pyrimidine-7- |
| | | | | | carbonyl)pyridin-2-yl)-4,5,6,7-tetrahydro- |
| | | | | | 1H-pyrazolo[4,3-c]pyridine-3-carboxamide |
| 434 | 0.082 | 2 | ND | ND | N-(4-(3,3-dimethylureido)benzyl)-1- |
| | | | | | isopropyl-5-(5-(1,2,3,4-tetrahydro-2,6- |
| | | | | | naphthyridine-2-carbonyl)pyridin-2-yl)- |
| | | | | | 4,5,6,7-tetrahydro-1H-pyrazolo[4,3- |
| | | | | | c]pyridine-3-carboxamide |
| 435 | 0.010 | 2 | ND | ND | N-(4-(3,3-dimethylureido)benzyl)-1- |
| | | | | | isopropyl-5-(5-(1,2,3,4- |
| | | | | | tetrahydroisoquinoline-2-carbonyl)pyridin-2- |
| | | | | | yl)-4,5,6,7-tetrahydro-1H-pyrazolo[4,3- |
| | | | | | c]pyridine-3-carboxamide |
| 436 | 0.022 | 2 | ND | ND | 5-(5-(6-cyano-1,2,3,4- |
| | | | | | tetrahydroisoquinoline-2-carbonyl)pyridin-2- |
| | | | | | yl)-N-(4-(3,3-dimethylureido)benzyl)-1- |
| | | | | | isopropyl-4,5,6,7-tetrahydro-1H- |
| | | | | | pyrazolo[4,3-c]pyridine-3-carboxamide |
| 437 | 0.045 | 3 | 1.053 | 1 | N-(4-(3,3-dimethylureido)benzyl)-1- |
| | | | | | isopropyl-5-(5-(1-isopropyl-4,5,6,7- |
| | | | | | tetrahydro-1H-pyrazolo[3,4-c]pyridine-6- |
| | | | | | carbonyl)pyridin-2-yl)-4,5,6,7-tetrahydro- |
| | | | | | 1H-pyrazolo[4,3-c]pyridine-3-carboxamide |
| 438 | 0.105 | 2 | ND | ND | N-(4-(3,3-dimethylureido)benzyl)-1- |
| | | | | | isopropyl-5-(5-(5,6,7,8- |
| | | | | | tetrahydroimidazo[1,5-a]pyrazine-7- |
| | | | | | carbonyl)pyridin-2-yl)-4,5,6,7-tetrahydro- |
| | | | | | 1H-pyrazolo[4,3-c]pyridine-3-carboxamide |
| 439 | 0.088 | 2 | ND | ND | N-(4-(3,3-dimethylureido)benzyl)-1- |
| | | | | | isopropyl-5-(5-(6-oxo-1,3,4,6-tetrahydro- |
| | | | | | 2H-pyrido[1,2-a]pyrazine-2- |
| | | | | | carbonyl)pyridin-2-yl)-4,5,6,7-tetrahydro- |
| | | | | | 1H-pyrazolo[4,3-c]pyridine-3-carboxamide |
| 440 | 0.016 | 2 | ND | ND | N-(4-(3,3-dimethylureido)benzyl)-1- |
| | | | | | isopropyl-5-(5-(6-methyl-2,3-dihydro-1H- |
| | | | | | pyrrolo[3,4-c]pyridine-2-carbonyl)pyridin-2- |
| | | | | | yl)-4,5,6,7-tetrahydro-1H-pyrazolo[4,3- |
| | | | | | c]pyridine-3-carboxamide |
| 441 | 0.002 | 2 | ND | ND | N-(4-(3,3-dimethylureido)benzyl)-1- |
| | | | | | isopropyl-5-(5-(4-methylisoindoline-2- |
| | | | | | carbonyl)pyridin-2-yl)-4,5,6,7-tetrahydro- |
| | | | | | 1H-pyrazolo[4,3-c]pyridine-3-carboxamide |
| 442 | 0.002 | 2 | ND | ND | N-(4-(3,3-dimethylureido)benzyl)-5-(5- |
| | | | | | (isoindoline-2-carbonyl)pyridin-2-yl)-1- |
| | | | | | isopropyl-4,5,6,7-tetrahydro-1H- |
| | | | | | pyrazolo[4,3-c]pyridine-3-carboxamide |
| 443 | 0.011 | 3 | 0.878 | 1 | N-(4-(3,3-dimethylureido)benzyl)-1- |
| | | | | | isopropyl-5-(5-(2-methoxy-5,6,7,8- |
| | | | | | tetrahydro-1,7-naphthyridine-7- |
| | | | | | carbonyl)pyridin-2-yl)-4,5,6,7-tetrahydro- |
| | | | | | 1H-pyrazolo[4,3-c]pyridine-3-carboxamide |
| 444\hat{ } | 0.158 | 2 | 1.375 | 2 | (S)-5-(5-(dimethylcarbamoyl)pyridin-2-yl)- |
| | | | | | N-(4-(3,3-dimethylureido)benzyl)-1,4- |
| | | | | | dimethyl-4,5,6,7-tetrahydro-1H- |
| | | | | | pyrazolo[4,3-c]pyridine-3-carboxamide |
| 445\hat{ } | 0.002 | 2 | 0.021 | 2 | (R)-5-(5-(dimethylcarbamoyl)pyridin-2-yl)- |
| | | | | | N-(4-(3,3-dimethylureido)benzyl)-1,4- |
| | | | | | dimethyl-4,5,6,7-tetrahydro-1H- |
| | | | | | pyrazolo[4,3-c]pyridine-3-carboxamide |
| 446\hat{ } | 0.015 | 2 | 0.080 | 2 | (6R,7R)-5-(5-(dimethylcarbamoyl)pyrimidin- |
| | | | | | 2-yl)-N-(4-(3,3-dimethylureido)benzyl)-1- |
| | | | | | ethyl-6,7-dimethyl-4,5,6,7-tetrahydro-1H- |
| | | | | | pyrazolo[4,3-c]pyridine-3-carboxamide |
| 447\hat{ } | 0.0004 | 2 | >0.413 | 2 | (6S,7S)-5-(5-(dimethylcarbamoyl)pyrimidin- |
| | | | | | 2-yl)-N-(4-(3,3-dimethylureido)benzyl)-1- |
| | | | | | ethyl-6,7-dimethyl-4,5,6,7-tetrahydro-1H- |
| | | | | | pyrazolo[4,3-c]pyridine-3-carboxamide |
| 448\hat{ } | 0.013 | 2 | >30.0 | 1 | (6R,7R)-5-(5-(dimethylcarbamoyl)pyrimidin- |
| | | | | | 2-yl)-N-(4-(3,3-dimethylureido)benzyl)-1- |
| | | | | | isopropyl-6,7-dimethyl-4,5,6,7-tetrahydro- |
| | | | | | 1H-pyrazolo[4,3-c]pyridine-3-carboxamide |
| 449\hat{ } | 0.0002 | 2 | 0.004 | 3 | (6S,7S)-5-(5-(dimethylcarbamoyl)pyrimidin- |
| | | | | | 2-yl)-N-(4-(3,3-dimethylureido)benzyl)-1- |
| | | | | | isopropyl-6,7-dimethyl-4,5,6,7-tetrahydro- |
| | | | | | 1H-pyrazolo[4,3-c]pyridine-3-carboxamide |
| 450 | 0.021 | 3 | 0.093 | 3 | (S)-N-(4-aminobenzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 451 | 0.003 | 2 | 0.047 | 2 | (S)-N-(4-(1H-imidazol-2-yl)benzyl)-5-(5- |
| | | | | | (dimethylcarbamoyl)pyrimidin-2-yl)-1- |
| | | | | | isopropyl-6-methyl-4,5,6,7-tetrahydro-1H- |
| | | | | | pyrazolo[4,3-c]pyridine-3-carboxamide |
| 452 | 0.003 | 2 | 0.016 | 2 | (S)-5-(5-(dimethylcarbamoyl)pyrimidin-2- |
| | | | | | yl)-N-(4-(3,3-dimethylureido)benzyl)-1- |
| | | | | | isopropyl-6-methyl-4,5,6,7-tetrahydro-1H- |
| | | | | | pyrazolo[4,3-c]pyridine-3-carboxamide |
| 453 | 2.150 | 2 | 12.110 | 2 | 5-(5-carbamoylpyridin-2-yl)-1-isopropyl-N- |
| | | | | | (4-(3-methylureido)benzyl)-4,5,6,7- |
| | | | | | tetrahydro-1H-pyrazolo[4,3-c]pyridine-3- |
| | | | | | carboxamide |
| 454 | 0.128 | 2 | 0.720 | 2 | 1-isopropyl-5-(5-(methylcarbamoyl)pyridin- |
| | | | | | 2-yl)-N-(4-(3-methylureido)benzyl)-4,5,6,7- |
| | | | | | tetrahydro-1H-pyrazolo[4,3-c]pyridine-3- |
| | | | | | carboxamide |
| 455 | 0.311 | 2 | ND | ND | N-(4-(3,3-dimethylureido)benzyl)-1- |
| | | | | | isopropyl-5-(5-(3-methyl-4,5,6,7-tetrahydro- |
| | | | | | [1,2,3]triazolo[1,5-a]pyrazine-5- |
| | | | | | carbonyl)pyridin-2-yl)-4,5,6,7-tetrahydro- |
| | | | | | 1H-pyrazolo[4,3-c]pyridine-3-carboxamide |
| 456 | 0.742 | 2 | 0.579 | 2 | 1-isopropyl-5-(5-(methylcarbamoyl)pyridin- |
| | | | | | 2-yl)-N-(4-ureidobenzyl)-4,5,6,7-tetrahydro- |
| | | | | | 1H-pyrazolo[4,3-c]pyridine-3-carboxamide |
| 457 | 5.961 | 1 | 5.010 | 2 | 5-(5-carbamoylpyridin-2-yl)-1-isopropyl-N- |
| | | | | | (4-ureidobenzyl)-4,5,6,7-tetrahydro-1H- |
| | | | | | pyrazolo[4,3-c]pyridine-3-carboxamide |
| 458 | 0.173 | 3 | >7.647 | 4 | (S)-N-(3-carbamoyl-2-fluorophenyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 459 | 0.0003 | 2 | 0.002 | 2 | rac-(R)-5-(5-(dimethylcarbamoyl)pyrimidin- |
| | | | | | 2-yl)-N-(4-(3,3-dimethylureido)benzyl)-1- |
| | | | | | isopropyl-7-(trifluoromethyl)-4,5,6,7- |
| | | | | | tetrahydro-1H-pyrazolo[4,3-c]pyridine-3- |
| | | | | | carboxamide |
| 460 | 0.0003 | 2 | 0.007 | 3 | rac-(R)-5-(5-(dimethylcarbamoyl)pyrimidin- |
| | | | | | 2-yl)-N-(4-(3,3-dimethylureido)benzyl)-1- |
| | | | | | ethyl-7-(trifluoromethyl)-4,5,6,7-tetrahydro- |
| | | | | | 1H-pyrazolo[4,3-c]pyridine-3-carboxamide |
| 461\hat{ } | 0.0002 | 2 | <0.001 | 3 | rel-(R or S)-5-(5- |
| | | | | | (dimethylcarbamoyl)pyrimidin-2-yl)-N-(4- |
| | | | | | (3,3-dimethylureido)benzyl)-1-isopropyl-7- |
| | | | | | (trifluoromethyl)-4,5,6,7-tetrahydro-1H- |
| | | | | | pyrazolo[4,3-c]pyridine-3-carboxamide |
| 462\hat{ } | 0.0387 | 2 | 0.376 | 2 | rel-(R or S)-5-(5- |
| | | | | | (dimethylcarbamoyl)pyrimidin-2-yl)-N-(4- |
| | | | | | (3,3-dimethylureido)benzyl)-1-isopropyl-7- |
| | | | | | (trifluoromethyl)-4,5,6,7-tetrahydro-1H- |
| | | | | | pyrazolo[4,3-c]pyridine-3-carboxamide |
| 463 | 0.0027 | 2 | 0.023 | 2 | rac-(R)-5-(5-(dimethylcarbamoyl)pyridin-2- |
| | | | | | yl)-1-ethyl-N-(4-(methylcarbamoyl)benzyl)- |
| | | | | | 7-(trifluoromethyl)-4,5,6,7-tetrahydro-1H- |
| | | | | | pyrazolo[4,3-c]pyridine-3-carboxamide |
| 464\hat{ } | 0.0002 | 2 | 0.002 | 4 | rel-(R or S)-5-(5- |
| | | | | | (dimethylcarbamoyl)pyrimidin-2-yl)-N-(4- |
| | | | | | (3,3-dimethylureido)benzyl)-1-ethyl-7- |
| | | | | | (trifluoromethyl)-4,5,6,7-tetrahydro-1H- |
| | | | | | pyrazolo[4,3-c]pyridine-3-carboxamide |
| 465\hat{ } | 0.0518 | 2 | 0.682 | 3 | rel-(R or S)-5-(5- |
| | | | | | (dimethylcarbamoyl)pyrimidin-2-yl)-N-(4- |
| | | | | | (3,3-dimethylureido)benzyl)-1-ethyl-7- |
| | | | | | (trifluoromethyl)-4,5,6,7-tetrahydro-1H- |
| | | | | | pyrazolo[4,3-c]pyridine-3-carboxamide |
| 466 | 0.002 | 2 | 0.018 | 2 | (S)-5-(5-(1,3-dimethyl-5-oxo-1,5-dihydro- |
| | | | | | 4H-1,2,4-triazol-4-yl)pyridin-2-yl)-N-(4-(3,3- |
| | | | | | dimethylureido)benzyl)-1-isopropyl-7- |
| | | | | | methyl-4,5,6,7-tetrahydro-1H-pyrazolo[4,3- |
| | | | | | c]pyridine-3-carboxamide |
| 467\hat{ } | 0.3150 | 2 | 2.501 | 3 | rel-(R or S)-5-(5- |
| | | | | | (dimethylcarbamoyl)pyridin-2-yl)-1-ethyl-N- |
| | | | | | (4-(methylcarbamoyl)benzyl)-7- |
| | | | | | (trifluoromethyl)-4,5,6,7-tetrahydro-1H- |
| | | | | | pyrazolo[4,3-c]pyridine-3-carboxamide |
| 468\hat{ } | 0.0022 | 2 | 0.012 | 4 | rel-(R or S)-5-(5- |
| | | | | | (dimethylcarbamoyl)pyridin-2-yl)-1-ethyl-N- |
| | | | | | (4-(methylcarbamoyl)benzyl)-7- |
| | | | | | (trifluoromethyl)-4,5,6,7-tetrahydro-1H- |
| | | | | | pyrazolo[4,3-c]pyridine-3-carboxamide |
| 469 | 5.108 | 1 | >4.161 | 5 | rac-(R)-N-(4-(3,3-dimethylureido)benzyl)-1- |
| | | | | | isopropyl-5-(5-(2-oxopyrrolidin-3-yl)pyridin- |
| | | | | | 2-yl)-4,5,6,7-tetrahydro-1H-pyrazolo[4,3- |
| | | | | | c]pyridine-3-carboxamide |
| 470 | 0.001 | 2 | 0.005 | 3 | (S)-5-(5-(dimethylcarbamoyl)pyridin-2-yl)-1- |
| | | | | | isopropyl-7-methyl-N-(4-(4- |
| | | | | | methylpiperazine-1-carboxamido)benzyl)- |
| | | | | | 4,5,6,7-tetrahydro-1H-pyrazolo[4,3- |
| | | | | | c]pyridine-3-carboxamide |
| 471 | 0.432 | 1 | 3.293 | 2 | (S)-N-(4-(1,3-dimethyl-5-oxo-1,5-dihydro- |
| | | | | | 4H-1,2,4-triazol-4-yl)benzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 472 | 0.041 | 1 | 0.120 | 2 | rac-(R)-5-(5-(dimethylcarbamoyl)pyridin-2- |
| | | | | | yl)-N-(4-(3,3-dimethylureido)benzyl)-7- |
| | | | | | hydroxy-1-isopropyl-4,5,6,7-tetrahydro-1H- |
| | | | | | pyrazolo[4,3-c]pyridine-3-carboxamide |
| 473 | 0.0003 | 1 | 0.005 | 2 | 5-(5-(dimethylcarbamoyl)pyridin-2-yl)-N-(4- |
| | | | | | (3,3-dimethylureido)benzyl)-7-hydroxy-1- |
| | | | | | isopropyl-4,5,6,7-tetrahydro-1H- |
| | | | | | pyrazolo[4,3-c]pyridine-3-carboxamide |
| 474\hat{ } | 0.040 | 1 | 0.085 | 2 | rel-(R or S)-5-(5- |
| | | | | | (dimethylcarbamoyl)pyridin-2-yl)-N-(4-(3,3- |
| | | | | | dimethylureido)benzyl)-7-hydroxy-1- |
| | | | | | isopropyl-4,5,6,7-tetrahydro-1H- |
| | | | | | pyrazolo[4,3-c]pyridine-3-carboxamide |
| 475\hat{ } | 0.072 | 1 | 0.215 | 2 | rel-(R or S)-5-(5- |
| | | | | | (dimethylcarbamoyl)pyridin-2-yl)-N-(4-(3,3- |
| | | | | | dimethylureido)benzyl)-7-hydroxy-1- |
| | | | | | isopropyl-4,5,6,7-tetrahydro-1H- |
| | | | | | pyrazolo[4,3-c]pyridine-3-carboxamide |
| 476 | 0.001 | 1 | 0.009 | 2 | (S)-1-ethyl-7-methyl-N-(4-(3- |
| | | | | | methylureido)benzyl)-5-(5-(2-oxopyrrolidin- |
| | | | | | 1-yl)pyridin-2-yl)-4,5,6,7-tetrahydro-1H- |
| | | | | | pyrazolo[4,3-c]pyridine-3-carboxamide |
| 477 | 0.0004 | 2 | 0.004 | 2 | (S)-5-(5-(6,7-dihydro-5H-pyrrolo[3,4- |
| | | | | | b]pyridine-6-carbonyl)pyridin-2-yl)-N-(4- |
| | | | | | (3,3-dimethylureido)benzyl)-1-ethyl-7- |
| | | | | | methyl-4,5,6,7-tetrahydro-1H-pyrazolo[4,3- |
| | | | | | c]pyridine-3-carboxamide |
| 478 | 0.002 | 2 | 0.017 | 2 | (S)-1-isopropyl-7-methyl-N-(4- |
| | | | | | (methylcarbamoyl)benzyl)-5-(5-(2- |
| | | | | | oxopyrrolidin-1-yl)pyridin-2-yl)-4,5,6,7- |
| | | | | | tetrahydro-1H-pyrazolo[4,3-c]pyridine-3- |
| | | | | | carboxamide |
| 479 | 0.004 | 2 | 0.015 | 2 | (S)-N-(4-carbamoylbenzyl)-1-isopropyl-7- |
| | | | | | methyl-5-(5-(2-oxopyrrolidin-1-yl)pyridin-2- |
| | | | | | yl)-4,5,6,7-tetrahydro-1H-pyrazolo[4,3- |
| | | | | | c]pyridine-3-carboxamide |
| 480 | 0.0003 | 2 | 0.003 | 2 | (S)-1-isopropyl-7-methyl-N-(4-(3- |
| | | | | | methylureido)benzyl)-5-(5-(2-oxopyrrolidin- |
| | | | | | 1-yl)pyridin-2-yl)-4,5,6,7-tetrahydro-1H- |
| | | | | | pyrazolo[4,3-c]pyridine-3-carboxamide |
| 481 | 0.001 | 2 | 0.014 | 2 | (S)-1-isopropyl-7-methyl-N-(4-(3- |
| | | | | | methylureido)benzyl)-5-(5-(2-oxopyrrolidin- |
| | | | | | 1-yl)pyrimidin-2-yl)-4,5,6,7-tetrahydro-1H- |
| | | | | | pyrazolo[4,3-c]pyridine-3-carboxamide |
| 482 | 0.007 | 2 | 0.025 | 3 | (S)-1-ethyl-7-methyl-N-(4-(3- |
| | | | | | methylureido)benzyl)-5-(5-(2-oxopyrrolidin- |
| | | | | | 1-yl)pyrimidin-2-yl)-4,5,6,7-tetrahydro-1H- |
| | | | | | pyrazolo[4,3-c]pyridine-3-carboxamide |
| 483 | 0.033 | 2 | 0.063 | 2 | (S)-N-(4-carbamoylbenzyl)-1-ethyl-7- |
| | | | | | methyl-5-(5-(2-oxopyrrolidin-1-yl)pyrimidin- |
| | | | | | 2-yl)-4,5,6,7-tetrahydro-1H-pyrazolo[4,3- |
| | | | | | c]pyridine-3-carboxamide |
| 484 | 0.026 | 2 | 0.108 | 2 | (S)-1-ethyl-7-methyl-N-(4- |
| | | | | | (methylcarbamoyl)benzyl)-5-(5-(2- |
| | | | | | oxopyrrolidin-1-yl)pyrimidin-2-yl)-4,5,6,7- |
| | | | | | tetrahydro-1H-pyrazolo[4,3-c]pyridine-3- |
| | | | | | carboxamide |
| 485 | 0.001 | 2 | 0.004 | 2 | (S)-5-(4-(dimethylcarbamoyl)phenyl)-1- |
| | | | | | isopropyl-7-methyl-N-(4-(4-(piperidin-4- |
| | | | | | yloxy)piperidine-1-carboxamido)benzyl)- |
| | | | | | 4,5,6,7-tetrahydro-1H-pyrazolo[4,3- |
| | | | | | c]pyridine-3-carboxamide |
| 486 | 0.002 | 2 | 0.011 | 2 | (S)-N-(4-(3,3-dimethylureido)benzyl)-1- |
| | | | | | isopropyl-7-methyl-5-(5-(2-oxopyrrolidin-1- |
| | | | | | yl)pyrimidin-2-yl)-4,5,6,7-tetrahydro-1H- |
| | | | | | pyrazolo[4,3-c]pyridine-3-carboxamide |
| 487 | 0.002 | 2 | 0.016 | 2 | (S)-N-(4-(1H-imidazol-2-yl)benzyl)-1- |
| | | | | | isopropyl-7-methyl-5-(5-(2-oxopyrrolidin-1- |
| | | | | | yl)pyrimidin-2-yl)-4,5,6,7-tetrahydro-1H- |
| | | | | | pyrazolo[4,3-c]pyridine-3-carboxamide |
| 488 | 0.003 | 2 | 0.027 | 2 | (S)-N-(4-(3,3-dimethylureido)benzyl)-1- |
| | | | | | ethyl-7-methyl-5-(5-(2-oxopyrrolidin-1- |
| | | | | | yl)pyrimidin-2-yl)-4,5,6,7-tetrahydro-1H- |
| | | | | | pyrazolo[4,3-c]pyridine-3-carboxamide |
| 489 | 0.018 | 2 | 0.017 | 2 | (S)-N-(4-carbamoylbenzyl)-1-isopropyl-7- |
| | | | | | methyl-5-(5-(2-oxopyrrolidin-1-yl)pyrimidin- |
| | | | | | 2-yl)-4,5,6,7-tetrahydro-1H-pyrazolo[4,3- |
| | | | | | c]pyridine-3-carboxamide |
| 490 | 0.013 | 2 | 0.050 | 2 | (S)-1-isopropyl-7-methyl-N-(4- |
| | | | | | (methylcarbamoyl)benzyl)-5-(5-(2- |
| | | | | | oxopyrrolidin-1-yl)pyrimidin-2-yl)-4,5,6,7- |
| | | | | | tetrahydro-1H-pyrazolo[4,3-c]pyridine-3- |
| | | | | | carboxamide |
| 491 | 0.007 | 2 | 0.034 | 2 | (S)-N-(4-aminobenzyl)-5-(5- |
| | | | | | (dimethylcarbamoyl)pyrimidin-2-yl)-1- |
| | | | | | isopropyl-7-methyl-4,5,6,7-tetrahydro-1H- |
| | | | | | pyrazolo[4,3-c]pyridine-3-carboxamide |
| 492 | 0.0005 | 2 | 0.004 | 2 | (S)-5-(5-(dimethylcarbamoyl)pyrimidin-2- |
| | | | | | yl)-N-(4-(3-(4-(3,3- |
| | | | | | dimethylureido)benzyl)ureido)benzyl)-1- |
| | | | | | isopropyl-7-methyl-4,5,6,7-tetrahydro-1H- |
| | | | | | pyrazolo[4,3-c]pyridine-3-carboxamide |
| 493\hat{ } | 0.028 | 2 | 0.203 | 2 | rel-(R or S)-7-(difluoromethyl)-5-(5- |
| | | | | | (dimethylcarbamoyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 494\hat{ } | 0.0005 | 2 | 0.004 | 2 | rel-(R or S)-7-(difluoromethyl)-5-(5- |
| | | | | | (dimethylcarbamoyl)pyridin-2-yl)-N-(4-(3,3- |
| | | | | | dimethylureido)benzyl)-1-isopropyl-4,5,6,7- |
| | | | | | tetrahydro-1H-pyrazolo[4,3-c]pyridine-3- |
| | | | | | carboxamide |
| 495 | 0.001 | 2 | 0.034 | 1 | (S)-5-(5-(6,7-dihydro-5H-pyrrolo[3,4- |
| | | | | | b]pyridine-6-carbonyl)pyrimidin-2-yl)-N-(4- |
| | | | | | (3,3-dimethylureido)benzyl)-1-ethyl-7- |
| | | | | | methyl-4,5,6,7-tetrahydro-1H-pyrazolo[4,3- |
| | | | | | c]pyridine-3-carboxamide |
| 496 | 0.004 | 2 | 0.016 | 2 | (S)-5-(5-(6,7-dihydro-5H-pyrrolo[3,4- |
| | | | | | b]pyridine-6-carbonyl)pyrimidin-2-yl)-1- |
| | | | | | isopropyl-7-methyl-N-(4- |
| | | | | | (methylcarbamoyl)benzyl)-4,5,6,7- |
| | | | | | tetrahydro-1H-pyrazolo[4,3-c]pyridine-3- |
| | | | | | carboxamide |
| 497 | 0.001 | 2 | 0.016 | 2 | ethyl (S)-(4-((5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3- |
| | | | | | carboxamido)methyl)phenyl)carbamate |
| 498 | 0.133 | 2 | 3.438 | 2 | (S)-2-(3-((4-(3,3- |
| | | | | | dimethylureido)benzyl)carbamoyl)-1- |
| | | | | | isopropyl-7-methyl-1,4,6,7-tetrahydro-5H- |
| | | | | | pyrazolo[4,3-c]pyridin-5-yl)pyrimidine-5- |
| | | | | | carboxylic acid |
| 499 | 0.0003 | 2 | 0.006 | 2 | (S)-5-(5-(dimethylcarbamoyl)pyridin-2-yl)- |
| | | | | | N-(4-(3-(4-(3,3- |
| | | | | | dimethylureido)benzyl)ureido)benzyl)-1- |
| | | | | | isopropyl-7-methyl-4,5,6,7-tetrahydro-1H- |
| | | | | | pyrazolo[4,3-c]pyridine-3-carboxamide |
| 500 | 0.011 | 2 | 0.104 | 2 | (S)-N-(4-(3,3-dimethylureido)benzyl)-5-(5- |
| | | | | | ((4-(3,3- |
| | | | | | dimethylureido)benzyl)carbamoyl)pyrimidin- |
| | | | | | 2-yl)-1-isopropyl-7-methyl-4,5,6,7- |
| | | | | | tetrahydro-1H-pyrazolo[4,3-c]pyridine-3- |
| | | | | | carboxamide |
| 501 | 0.007 | 2 | 0.050 | 2 | (S)-N-(4-(3,3-dimethylureido)benzyl)-5-(5- |
| | | | | | ((4-(3,3- |
| | | | | | dimethylureido)benzyl)carbamoyl)pyridin-2- |
| | | | | | yl)-1-isopropyl-7-methyl-4,5,6,7-tetrahydro- |
| | | | | | 1H-pyrazolo[4,3-c]pyridine-3-carboxamide |
| 502 | 0.813 | 2 | 7.553 | 2 | (S)-N-(4-(3,3-dimethylureido)benzyl)-1,7- |
| | | | | | dimethyl-5-(5-(1-methyl-1H-imidazol-2- |
| | | | | | yl)pyrimidin-2-yl)-4,5,6,7-tetrahydro-1H- |
| | | | | | pyrazolo[4,3-c]pyridine-3-carboxamide |
| 503 | 0.001 | 2 | 0.013 | 2 | ethyl (S)-(4-((5-(5- |
| | | | | | (dimethylcarbamoyl)pyrimidin-2-yl)-1- |
| | | | | | isopropyl-7-methyl-4,5,6,7-tetrahydro-1H- |
| | | | | | pyrazolo[4,3-c]pyridine-3- |
| | | | | | carboxamido)methyl)phenyl)carbamate |
| 504 | 0.874 | 2 | >21.2 | 2 | (S)-6-(3-((4-(3,3- |
| | | | | | dimethylureido)benzyl)carbamoyl)-1- |
| | | | | | isopropyl-7-methyl-1,4,6,7-tetrahydro-5H- |
| | | | | | pyrazolo[4,3-c]pyridin-5-yl)nicotinic acid |
| 505 | 7.895 | 1 | >30.0 | 1 | rac-(R)-N-(4-(3,3-dimethylureido)benzyl)-1- |
| | | | | | isopropyl-5-(5-(1-methyl-2-oxopyrrolidin-3- |
| | | | | | yl)pyridin-2-yl)-4,5,6,7-tetrahydro-1H- |
| | | | | | pyrazolo[4,3-c]pyridine-3-carboxamide |
| 506\hat{ } | 0.002 | 2 | 0.014 | 3 | (R)-5-(5-(dimethylcarbamoyl)pyridin-2-yl)- |
| | | | | | N-(4-(3,3-dimethylureido)benzyl)-1- |
| | | | | | isopropyl-4-methyl-4,5,6,7-tetrahydro-1H- |
| | | | | | pyrazolo[4,3-c]pyridine-3-carboxamide |
| 507\hat{ } | 0.136 | 2 | 1.649 | 2 | (S)-5-(5-(dimethylcarbamoyl)pyridin-2-yl)- |
| | | | | | N-(4-(3,3-dimethylureido)benzyl)-1- |
| | | | | | isopropyl-4-methyl-4,5,6,7-tetrahydro-1H- |
| | | | | | pyrazolo[4,3-c]pyridine-3-carboxamide |
| 508\hat{ } | 0.007 | 2 | 0.020 | 3 | (R)-5-(5-(dimethylcarbamoyl)pyrimidin-2- |
| | | | | | yl)-N-(4-(3,3-dimethylureido)benzyl)-1- |
| | | | | | isopropyl-4-methyl-4,5,6,7-tetrahydro-1H- |
| | | | | | pyrazolo[4,3-c]pyridine-3-carboxamide |
| 509\hat{ } | 3.316 | 1 | 8.170 | 2 | (S)-5-(5-(dimethylcarbamoyl)pyrimidin-2- |
| | | | | | yl)-N-(4-(3,3-dimethylureido)benzyl)-1- |
| | | | | | isopropyl-4-methyl-4,5,6,7-tetrahydro-1H- |
| | | | | | pyrazolo[4,3-c]pyridine-3-carboxamide |
| 510\hat{ } | 1.517 | 2 | 8.832 | 2 | (S)-5-(5-(dimethylcarbamoyl)pyrimidin-2- |
| | | | | | yl)-N-(4-(3,3-dimethylureido)benzyl)-1- |
| | | | | | ethyl-4-methyl-4,5,6,7-tetrahydro-1H- |
| | | | | | pyrazolo[4,3-c]pyridine-3-carboxamide |
| 511\hat{ } | 0.006 | 2 | 0.029 | 2 | (R)-5-(5-(dimethylcarbamoyl)pyrimidin-2- |
| | | | | | yl)-N-(4-(3,3-dimethylureido)benzyl)-1- |
| | | | | | ethyl-4-methyl-4,5,6,7-tetrahydro-1H- |
| | | | | | pyrazolo[4,3-c]pyridine-3-carboxamide |
| 512\hat{ } | 0.240 | 2 | 2.508 | 2 | (S)-5-(5-(dimethylcarbamoyl)pyridin-2-yl)- |
| | | | | | N-(4-(3,3-dimethylureido)benzyl)-1-ethyl-4- |
| | | | | | methyl-4,5,6,7-tetrahydro-1H-pyrazolo[4,3- |
| | | | | | c]pyridine-3-carboxamide |
| 513\hat{ } | 0.003 | 2 | 0.018 | 2 | (R)-5-(5-(dimethylcarbamoyl)pyridin-2-yl)- |
| | | | | | N-(4-(3,3-dimethylureido)benzyl)-1-ethyl-4- |
| | | | | | methyl-4,5,6,7-tetrahydro-1H-pyrazolo[4,3- |
| | | | | | c]pyridine-3-carboxamide |
| 514 | 0.001 | 2 | 0.015 | 2 | (S)-1-isopropyl-7-methyl-5-(5-(1-methyl-1H- |
| | | | | | imidazol-2-yl)pyridin-2-yl)-N-(4-(3- |
| | | | | | methylureido)benzyl)-4,5,6,7-tetrahydro- |
| | | | | | 1H-pyrazolo[4,3-c]pyridine-3-carboxamide |
| 515 | 0.001 | 2 | 0.006 | 2 | (S)-1-isopropyl-7-methyl-5-(5-(1-methyl-1H- |
| | | | | | imidazol-2-yl)pyrimidin-2-yl)-N-(4-(3- |
| | | | | | methylureido)benzyl)-4,5,6,7-tetrahydro- |
| | | | | | 1H-pyrazolo[4,3-c]pyridine-3-carboxamide |
| 516 | 0.001 | 2 | 0.005 | 2 | (S)-1-isopropyl-7-methyl-5-(5-(1-methyl-1H- |
| | | | | | imidazol-2-yl)pyridin-2-yl)-N-(4- |
| | | | | | ureidobenzyl)-4,5,6,7-tetrahydro-1H- |
| | | | | | pyrazolo[4,3-c]pyridine-3-carboxamide |
| 517 | 0.009 | 2 | 0.033 | 2 | (S)-N-(4-carbamoylbenzyl)-1-isopropyl-7- |
| | | | | | methyl-5-(5-(2-oxooxazolidin-3-yl)pyridin-2- |
| | | | | | yl)-4,5,6,7-tetrahydro-1H-pyrazolo[4,3- |
| | | | | | c]pyridine-3-carboxamide |
| 518 | 0.002 | 2 | 0.071 | 2 | (S)-1-isopropyl-7-methyl-N-(4-(4-methyl- |
| | | | | | 1H-imidazol-2-yl)benzyl)-5-(5-(2- |
| | | | | | oxooxazolidin-3-yl)pyridin-2-yl)-4,5,6,7- |
| | | | | | tetrahydro-1H-pyrazolo[4,3-c]pyridine-3- |
| | | | | | carboxamide |
| 519 | 0.008 | 2 | 0.219 | 1 | (S)-1-isopropyl-7-methyl-N-(4-(4-methyl- |
| | | | | | 1H-imidazol-2-yl)benzyl)-5-(5-(2- |
| | | | | | oxooxazolidin-3-yl)pyrimidin-2-yl)-4,5,6,7- |
| | | | | | tetrahydro-1H-pyrazolo[4,3-c]pyridine-3- |
| | | | | | carboxamide |
| 520 | 0.001 | 2 | 0.022 | 1 | (S)-1-isopropyl-7-methyl-N-(4-(3- |
| | | | | | methylureido)benzyl)-5-(5-(2-oxooxazolidin- |
| | | | | | 3-yl)pyridin-2-yl)-4,5,6,7-tetrahydro-1H- |
| | | | | | pyrazolo[4,3-c]pyridine-3-carboxamide |
| 521 | 0.002 | 2 | 0.034 | 2 | (S)-5-(5-(1,3-dimethyl-5-oxo-1,5-dihydro- |
| | | | | | 4H-1,2,4-triazol-4-yl)pyridin-2-yl)-1- |
| | | | | | isopropyl-7-methyl-N-(4-(3- |
| | | | | | methylureido)benzyl)-4,5,6,7-tetrahydro- |
| | | | | | 1H-pyrazolo[4,3-c]pyridine-3-carboxamide |
| 524 | 0.0001 | 1 | 0.006 | 3 | (4RS,7S)-5-(5-(dimethylcarbamoyl)pyridin- |
| | | | | | 2-yl)-N-(4-(3,3-dimethylureido)benzyl)-1- |
| | | | | | isopropyl-4,7-dimethyl-4,5,6,7-tetrahydro- |
| | | | | | 1H-pyrazolo[4,3-c]pyridine-3-carboxamide |
| 525 | 0.002 | 4 | 0.007 | 6 | (S)-5-(4-(dimethylcarbamoyl)phenyl)-1- |
| | | | | | isopropyl-7-methyl-N-(4-(piperazine-1- |
| | | | | | carboxamido)benzyl)-4,5,6,7-tetrahydro- |
| | | | | | 1H-pyrazolo[4,3-c]pyridine-3-carboxamide |
| 526 | 4.009 | 1 | 2.037 | 2 | 5-(5-(dimethylcarbamoyl)pyridin-2-yl)-N-(4- |
| | | | | | (3-methylureido)benzyl)-4,5,6,7-tetrahydro- |
| | | | | | 1H-pyrazolo[4,3-c]pyridine-3-carboxamide |
| 527\hat{ } | 8.872 | 1 | >25.1 | 3 | (S)-5-(5-(dimethylcarbamoyl)pyridin-2-yl)- |
| | | | | | N-(4-(3,3-dimethylureido)benzyl)-4-ethyl-1- |
| | | | | | isopropyl-4,5,6,7-tetrahydro-1H- |
| | | | | | pyrazolo[4,3-c]pyridine-3-carboxamide |
| 528\hat{ } | 0.017 | 2 | 0.067 | 3 | (S)-5-(5-(dimethylcarbamoyl)pyridin-2-yl)- |
| | | | | | N-(4-(3,3-dimethylureido)benzyl)-4-ethyl-1- |
| | | | | | isopropyl-4,5,6,7-tetrahydro-1H- |
| | | | | | pyrazolo[4,3-c]pyridine-3-carboxamide |
| 529\hat{ } | 0.0001 | 1 | 0.004 | 2 | (4R,7S)-5-(5-(dimethylcarbamoyl)pyridin-2- |
| | | | | | yl)-N-(4-(3,3-dimethylureido)benzyl)-1- |
| | | | | | isopropyl-4,7-dimethyl-4,5,6,7-tetrahydro- |
| | | | | | 1H-pyrazolo[4,3-c]pyridine-3-carboxamide |
| 530 | 0.0002 | 2 | 0.006 | 2 | (S)-N-(4-(3,3-dimethylureido)benzyl)-1- |
| | | | | | isopropyl-7-methyl-5-(5-(2-oxopyrrolidin-1- |
| | | | | | yl)pyridin-2-yl)-4,5,6,7-tetrahydro-1H- |
| | | | | | pyrazolo[4,3-c]pyridine-3-carboxamide |
| 531 | 0.001 | 2 | 0.014 | 2 | rac-(R)-5-(5-(dimethylcarbamoyl)pyrimidin- |
| | | | | | 2-yl)-N-(4-(3,3-dimethylureido)benzyl)-1- |
| | | | | | ethyl-7-methyl-4,5,6,7-tetrahydro-1H- |
| | | | | | pyrazolo[4,3-c]pyridine-3-carboxamide |
| 532 | 0.001 | 2 | 0.004 | 2 | rac-(R)-5-(5-(dimethylcarbamoyl)pyrimidin- |
| | | | | | 2-yl)-N-(4-(3,3-dimethylureido)benzyl)-1- |
| | | | | | isopropyl-7-methyl-4,5,6,7-tetrahydro-1H- |
| | | | | | pyrazolo[4,3-c]pyridine-3-carboxamide |
| 533 | 0.005 | 2 | 0.018 | 2 | (S)-5-(5-(dimethylcarbamoyl)pyrimidin-2- |
| | | | | | yl)-N-((5-(3,3-dimethylureido)pyridin-2- |
| | | | | | yl)methyl)-1-isopropyl-6-methyl-4,5,6,7- |
| | | | | | tetrahydro-1H-pyrazolo[4,3-c]pyridine-3- |
| | | | | | carboxamide |
| 534 | 0.033 | 2 | 0.155 | 2 | (S)-5-(5-(dimethylcarbamoyl)pyrimidin-2- |
| | | | | | yl)-1-isopropyl-6-methyl-N-(4- |
| | | | | | (methylcarbamoyl)benzyl)-4,5,6,7- |
| | | | | | tetrahydro-1H-pyrazolo[4,3-c]pyridine-3- |
| | | | | | carboxamide |
| 535 | 0.005 | 2 | 0.013 | 3 | (S)-5-(5-(dimethylcarbamoyl)pyridin-2-yl)- |
| | | | | | N-((5-(3,3-dimethylureido)pyridin-2- |
| | | | | | yl)methyl)-1-isopropyl-6-methyl-4,5,6,7- |
| | | | | | tetrahydro-1H-pyrazolo[4,3-c]pyridine-3- |
| | | | | | carboxamide |
| 536 | 0.004 | 2 | 0.031 | 2 | (S)-N-(4-(1H-imidazol-2-yl)benzyl)-5-(5- |
| | | | | | (dimethylcarbamoyl)pyridin-2-yl)-1- |
| | | | | | isopropyl-6-methyl-4,5,6,7-tetrahydro-1H- |
| | | | | | pyrazolo[4,3-c]pyridine-3-carboxamide |
| 537 | 0.001 | 2 | 0.010 | 2 | rac-(R)-7-(4-(dimethylcarbamoyl)phenyl)-N- |
| | | | | | (4-(3,3-dimethylureido)benzyl)-3-isopropyl- |
| | | | | | 5-methyl-5,6,7,8-tetrahydroimidazo[1,5- |
| | | | | | a]lpyrazine-1-carboxamide |
| 538\hat{ } | 0.0002 | 2 | 0.005 | 2 | rel-(R or S)-7-(4- |
| | | | | | (dimethylcarbamoyl)phenyl)-N-(4-(3,3- |
| | | | | | dimethylureido)benzyl)-3-isopropyl-5- |
| | | | | | methyl-5,6,7,8-tetrahydroimidazo[1,5- |
| | | | | | a]lpyrazine-1-carboxamide |
| 539\hat{ } | 0.013 | 2 | 0.072 | 2 | rel-(R or S)-7-(4- |
| | | | | | (dimethylcarbamoyl)phenyl)-N-(4-(3,3- |
| | | | | | dimethylureido)benzyl)-3-isopropyl-5- |
| | | | | | methyl-5,6,7,8-tetrahydroimidazo[1,5- |
| | | | | | a]lpyrazine-1-carboxamide |
| 540 | 0.027 | 2 | 0.076 | 2 | (S)-5-(5-(dimethylcarbamoyl)pyridin-2-yl)-1- |
| | | | | | isopropyl-6-methyl-N-(4- |
| | | | | | (methylcarbamoyl)benzyl)-4,5,6,7- |
| | | | | | tetrahydro-1H-pyrazolo[4,3-c]pyridine-3- |
| | | | | | carboxamide |
| 546 | 0.0003 | 2 | 0.005 | 5 | rac-(R)-5-(5-(dimethylcarbamoyl)pyridin-2- |
| | | | | | yl)-N-(4-(3,3-dimethylureido)benzyl)-1- |
| | | | | | isopropyl-7-(trifluoromethyl)-4,5,6,7- |
| | | | | | tetrahydro-1H-pyrazolo[4,3-c]pyridine-3- |
| | | | | | carboxamide |
| 547\hat{ } | 0.0002 | 2 | 0.003 | 5 | rel-(R or S)-5-(5- |
| | | | | | (dimethylcarbamoyl)pyridin-2-yl)-N-(4-(3,3- |
| | | | | | dimethylureido)benzyl)-1-isopropyl-7- |
| | | | | | (trifluoromethyl)-4,5,6,7-tetrahydro-1H- |
| | | | | | pyrazolo[4,3-c]pyridine-3-carboxamide |
| 548\hat{ } | 0.015 | 2 | 0.199 | 3 | rel-(R or S)-5-(5- |
| | | | | | (dimethylcarbamoyl)pyridin-2-yl)-N-(4-(3,3- |
| | | | | | dimethylureido)benzyl)-1-isopropyl-7- |
| | | | | | (trifluoromethyl)-4,5,6,7-tetrahydro-1H- |
| | | | | | pyrazolo[4,3-c]pyridine-3-carboxamide |
| 549\hat{ } | 0.099 | 3 | 1.102 | 2 | (R)-N-(4-(1H-imidazol-2-yl)benzyl)-5-(5- |
| | | | | | (dimethylcarbamoyl)pyrimidin-2-yl)-1- |
| | | | | | isopropyl-7-methyl-4,5,6,7-tetrahydro-1H- |
| | | | | | pyrazolo[4,3-c]pyridine-3-carboxamide |
| 550\hat{ } | 0.011 | 3 | 0.085 | 3 | (R)-5-(5-(dimethylcarbamoyl)pyridin-2-yl)- |
| | | | | | N-(4-(3,3-dimethylureido)benzyl)-7-methyl- |
| | | | | | 1-(2,2,2-trifluoroethyl)-4,5,6,7-tetrahydro- |
| | | | | | 1H-pyrazolo[4,3-c]pyridine-3-carboxamide |
| 551\hat{ } | 0.012 | 3 | 0.104 | 3 | (R)-5-(5-(dimethylcarbamoyl)pyridin-2-yl)- |
| | | | | | N-(4-(3,3-dimethylureido)benzyl)-7-methyl- |
| | | | | | 1-(1-methylcyclopropyl)-4,5,6,7-tetrahydro- |
| | | | | | 1H-pyrazolo[4,3-c]pyridine-3-carboxamide |
| 552\hat{ } | 0.034 | 3 | 0.087 | 3 | (R)-5-(5-(dimethylcarbamoyl)pyrimidin-2- |
| | | | | | yl)-N-(4-(3,3-dimethylureido)benzyl)-1- |
| | | | | | isopropyl-7-methyl-4,5,6,7-tetrahydro-1H- |
| | | | | | pyrazolo[4,3-c]pyridine-3-carboxamide |
| 553\hat{ } | 0.004 | 2 | 0.018 | 3 | (R)-5-(5-(dimethylcarbamoyl)pyridin-2-yl)- |
| | | | | | N-(4-(3,3-dimethylureido)benzyl)-7-methyl- |
| | | | | | 1-((RS)-1,1,1-trifluoropropan-2-yl)-4,5,6,7- |
| | | | | | tetrahydro-1H-pyrazolo[4,3-c]pyridine-3- |
| | | | | | carboxamide |
| 555 | 0.005 | 2 | 0.052 | 2 | 5′-(5-(dimethylcarbamoyl)pyridin-2-yl)-N-(4- |
| | | | | | (3,3-dimethylureido)benzyl)-1′-ethyl- |
| | | | | | 1′,4′,5′,6′-tetrahydrospiro[cyclopropane-1,7′- |
| | | | | | pyrazolo[4,3-c]pyridine]-3′-carboxamide |
| 556\hat{ } | 0.058 | 3 | 0.196 | 3 | (R)-5-(5-(dimethylcarbamoyl)pyridin-2-yl)- |
| | | | | | N-(4-(3,3-dimethylureido)benzyl)-7-methyl- |
| | | | | | 1-(tetrahydro-2H-pyran-4-yl)-4,5,6,7- |
| | | | | | tetrahydro-1H-pyrazolo[4,3-c]pyridine-3- |
| | | | | | carboxamide |
| 557\hat{ } | 0.015 | 3 | 0.204 | 3 | (R)-1-cyclobutyl-5-(5- |
| | | | | | (dimethylcarbamoyl)pyridin-2-yl)-N-(4-(3,3- |
| | | | | | dimethylureido)benzyl)-7-methyl-4,5,6,7- |
| | | | | | tetrahydro-1H-pyrazolo[4,3-c]pyridine-3- |
| | | | | | carboxamide |
| 562 | 0.003 | 2 | 0.008 | 2 | (S)-1-isopropyl-7-methyl-5-(5-(1-methyl-1H- |
| | | | | | imidazol-2-yl)pyrimidin-2-yl)-N-(4- |
| | | | | | ureidobenzyl)-4,5,6,7-tetrahydro-1H- |
| | | | | | pyrazolo[4,3-c]pyridine-3-carboxamide |
| 563 | 0.202 | 3 | ND | ND | N-(4-(3,3-dimethylureido)benzyl)-5-(5-(4- |
| | | | | | (ethylsulfonyl)piperazine-1- |
| | | | | | carbonyl)pyridin-2-yl)-1-isopropyl-4,5,6,7- |
| | | | | | tetrahydro-1H-pyrazolo[4,3-c]pyridine-3- |
| | | | | | carboxamide |
| 564 | 0.223 | 2 | ND | ND | N-(4-(3,3-dimethylureido)benzyl)-1- |
| | | | | | isopropyl-5-(5-(1-methyl-2-oxo-1,8- |
| | | | | | diazaspiro[4.5]decane-8-carbonyl)pyridin-2- |
| | | | | | yl)-4,5,6,7-tetrahydro-1H-pyrazolo[4,3- |
| | | | | | c]pyridine-3-carboxamide |
| 565 | 0.005 | 1 | ND | ND | (S)-N-(4-(3,3-dimethylureido)benzyl)-1- |
| | | | | | isopropyl-5-(4-(((1-(2- |
| | | | | | methoxyethyl)piperidin-4- |
| | | | | | yl)methyl)(methyl)carbamoyl)phenyl)-7- |
| | | | | | methyl-4,5,6,7-tetrahydro-1H-pyrazolo[4,3- |
| | | | | | c]pyridine-3-carboxamide |
| 566 | 0.002 | 2 | 0.015 | 3 | (S)-5-(4-(dimethylcarbamoyl)phenyl)-N-(3- |
| | | | | | fluoro-4-(piperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 567 | 0.001 | 2 | 0.013 | 3 | (S)-5-(4-(dimethylcarbamoyl)phenyl)-1- |
| | | | | | isopropyl-7-methyl-N-(4-(4-methyl-1,4- |
| | | | | | diazepane-1-carboxamido)benzyl)-4,5,6,7- |
| | | | | | tetrahydro-1H-pyrazolo[4,3-c]pyridine-3- |
| | | | | | carboxamide |
| 568 | 0.002 | 2 | 0.013 | 3 | (S)-5-(4-(dimethylcarbamoyl)phenyl)-N-(3- |
| | | | | | fluoro-4-((R)-3-(methylamino)piperidine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 569 | 0.004 | 2 | 0.020 | 3 | (S)-5-(4-(dimethylcarbamoyl)phenyl)-N-(3- |
| | | | | | fluoro-4-((S)-3-(methylamino)piperidine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 570 | 0.003 | 2 | 0.027 | 2 | (S)-N-(4-((S)-3-amino-3-methylpyrrolidine- |
| | | | | | 1-carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 571 | 0.004 | 2 | 0.020 | 2 | (S)-5-(4-(dimethylcarbamoyl)phenyl)-1- |
| | | | | | isopropyl-7-methyl-N-(4-((S)-3- |
| | | | | | (methylamino)pyrrolidine-1- |
| | | | | | carboxamido)benzyl)-4,5,6,7-tetrahydro- |
| | | | | | 1H-pyrazolo[4,3-c]pyridine-3-carboxamide |
| 572 | 0.003 | 2 | 0.020 | 2 | (S)-N-(4-((R)-3-amino-3-methylpyrrolidine- |
| | | | | | 1-carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 573 | 0.001 | 2 | 0.011 | 2 | (S)-N-(4-((1RS,5SR)-3,8- |
| | | | | | diazabicyclo[3.2.1]octane-3-carboxamido)- |
| | | | | | 3-fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 574 | 0.0005 | 3 | 0.011 | 2 | (S)-5-(4-(dimethylcarbamoyl)phenyl)-1- |
| | | | | | isopropyl-7-methyl-N-(4-((R)- |
| | | | | | octahydropyrrolo[1,2-a]pyrazine-2- |
| | | | | | carboxamido)benzyl)-4,5,6,7-tetrahydro- |
| | | | | | 1H-pyrazolo[4,3-c]pyridine-3-carboxamide |
| 575 | 0.001 | 3 | 0.010 | 2 | (S)-5-(4-(dimethylcarbamoyl)phenyl)-1- |
| | | | | | isopropyl-7-methyl-N-(4-((S)- |
| | | | | | octahydropyrrolo[1,2-a]pyrazine-2- |
| | | | | | carboxamido)benzyl)-4,5,6,7-tetrahydro- |
| | | | | | 1H-pyrazolo[4,3-c]pyridine-3-carboxamide |
| 576 | 0.004 | 2 | 0.019 | 2 | (S)-N-(4-(1,4-diazepane-1-carboxamido)-3- |
| | | | | | fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 577 | 0.001 | 3 | 0.004 | 2 | (S)-5-(4-(dimethylcarbamoyl)phenyl)-1- |
| | | | | | isopropyl-N-(4-((R)-3- |
| | | | | | (methoxymethyl)piperazine-1- |
| | | | | | carboxamido)benzyl)-7-methyl-4,5,6,7- |
| | | | | | tetrahydro-1H-pyrazolo[4,3-c]pyridine-3- |
| | | | | | carboxamide |
| 578 | 0.0005 | 2 | 0.005 | 2 | (S)-5-(4-(dimethylcarbamoyl)phenyl)-1- |
| | | | | | isopropyl-N-(4-((S)-3- |
| | | | | | (methoxymethyl)piperazine-1- |
| | | | | | carboxamido)benzyl)-7-methyl-4,5,6,7- |
| | | | | | tetrahydro-1H-pyrazolo[4,3-c]pyridine-3- |
| | | | | | carboxamide |
| 579 | 0.0004 | 2 | 0.008 | 2 | (S)-N-(4-(3-(2,2- |
| | | | | | difluoroethyl)ureido)benzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 580 | 0.001 | 2 | 0.011 | 2 | (S)-5-(4-(dimethylcarbamoyl)phenyl)-N-(3- |
| | | | | | fluoro-4-(4-methylpiperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 581 | 0.003 | 2 | 0.024 | 2 | (S)-5-(4-(dimethylcarbamoyl)phenyl)-1- |
| | | | | | isopropyl-7-methyl-N-(4-(3-methyl-3- |
| | | | | | (methylamino)azetidine-1- |
| | | | | | carboxamido)benzyl)-4,5,6,7-tetrahydro- |
| | | | | | 1H-pyrazolo[4,3-c]pyridine-3-carboxamide |
| 583 | 0.001 | 2 | 0.017 | 2 | (S)-5-(5-(dimethylcarbamoyl)pyridin-2-yl)-1- |
| | | | | | isopropyl-7-methyl-N-(4-(pyrrolidine-1- |
| | | | | | carboxamido)benzyl)-4,5,6,7-tetrahydro- |
| | | | | | 1H-pyrazolo[4,3-c]pyridine-3-carboxamide |
| 584 | 0.001 | 2 | 0.012 | 2 | (S)-5-(5-(dimethylcarbamoyl)pyridin-2-yl)- |
| | | | | | N-(4-(3-fluoroazetidine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 585 | 0.006 | 2 | 0.037 | 2 | (S)-5-(4-(dimethylcarbamoyl)phenyl)-1- |
| | | | | | isopropyl-7-methyl-N-(4-(1- |
| | | | | | methylpiperidine-4-carboxamido)benzyl)- |
| | | | | | 4,5,6,7-tetrahydro-1H-pyrazolo[4,3- |
| | | | | | c]pyridine-3-carboxamide |
| 586 | 0.006 | 2 | 0.058 | 2 | (S)-5-(4-(dimethylcarbamoyl)phenyl)-N-(4- |
| | | | | | (4-fluoro-1-methylpiperidine-4- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 587 | 0.014 | 2 | 0.122 | 2 | (S)-5-(5-(dimethylcarbamoyl)pyridin-2-yl)- |
| | | | | | N-(3-fluoro-4-(1-methylpiperidine-4- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 588 | 0.075 | 2 | 0.875 | 2 | (S)-5-(4-(dimethylcarbamoyl)phenyl)-N-(5- |
| | | | | | fluoro-2-methoxy-4-(piperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 589 | 0.003 | 2 | 0.019 | 2 | (S)-5-(4-(dimethylcarbamoyl)phenyl)-N-(2- |
| | | | | | fluoro-4-(piperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 590 | 0.0004 | 2 | 0.010 | 2 | (S)-N-(4-(3,3-difluoropyrrolidine-1- |
| | | | | | carboxamido)benzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 591 | 0.001 | 2 | 0.010 | 2 | (S)-N-(4-(azetidine-1-carboxamido)benzyl)- |
| | | | | | 5-(5-(dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 592 | 0.001 | 2 | 0.009 | 2 | (S)-5-(5-(dimethylcarbamoyl)pyridin-2-yl)- |
| | | | | | N-(4-(4-hydroxy-4-methylpiperidine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 593 | 0.001 | 2 | 0.012 | 2 | (S)-N-(4-(4-cyano-4-methylpiperidine-1- |
| | | | | | carboxamido)benzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 594 | 0.002 | 2 | 0.025 | 2 | (S)-1-isopropyl-7-methyl-N-(4-(4- |
| | | | | | methylpiperazine-1-carboxamido)benzyl)-5- |
| | | | | | (4-(2-oxopyridin-1(2H)-yl)phenyl)-4,5,6,7- |
| | | | | | tetrahydro-1H-pyrazolo[4,3-c]pyridine-3- |
| | | | | | carboxamide |
| 595 | 0.064 | 2 | 0.083 | 2 | (S)-5-(4-(dimethylcarbamoyl)phenyl)-N-(3- |
| | | | | | fluoro-4-(2,6-diazaspiro[3.3]heptane-2- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 596 | 0.003 | 2 | 0.045 | 2 | (S)-N-(3-chloro-4-(piperazine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 597 | 0.004 | 2 | 0.028 | 2 | (S)-5-(4-(dimethylcarbamoyl)phenyl)-N-(3- |
| | | | | | fluoro-4-(3-(methylamino)azetidine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 598 | 0.001 | 2 | 0.013 | 2 | (S)-N-(4-(3-(2,2-difluoroethyl)-3- |
| | | | | | methylureido)benzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 599 | 0.001 | 2 | 0.012 | 2 | (S)-N-(4-(3,3-difluoroazetidine-1- |
| | | | | | carboxamido)benzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 601 | 0.312 | 2 | 2.102 | 2 | (S)-5-(5-(dimethylcarbamoyl)pyridin-2-yl)-1- |
| | | | | | isopropyl-7-methyl-N-(4-((1- |
| | | | | | methylpiperidin-4-yl)carbamoyl)benzyl)- |
| | | | | | 4,5,6,7-tetrahydro-1H-pyrazolo[4,3- |
| | | | | | c]pyridine-3-carboxamide |
| 602 | 0.001 | 2 | 0.014 | 2 | (S)-5-(5-(dimethylcarbamoyl)pyridin-2-yl)- |
| | | | | | N-(3-fluoro-4-(1-methyl-1,7- |
| | | | | | diazaspiro[3.5]nonane-7- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 603 | 0.003 | 2 | 0.031 | 2 | (S)-5-(5-(dimethylcarbamoyl)pyridin-2-yl)- |
| | | | | | N-(3-fluoro-4-(6-methyl-2,6- |
| | | | | | diazaspiro[3.4]octane-2- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 604 | 0.004 | 2 | 0.029 | 2 | (S)-5-(5-(dimethylcarbamoyl)pyridin-2-yl)- |
| | | | | | N-(3-fluoro-4-(6-methyl-2,6- |
| | | | | | diazaspiro[3.3]heptane-2- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 605 | 0.004 | 2 | 0.037 | 2 | (S)-5-(4-(dimethylcarbamoyl)phenyl)-1- |
| | | | | | isopropyl-7-methyl-N-(4- |
| | | | | | (methylcarbamoyl)benzyl)-4,5,6,7- |
| | | | | | tetrahydro-1H-pyrazolo[4,3-c]pyridine-3- |
| | | | | | carboxamide |
| 606 | 0.001 | 2 | 0.009 | 2 | (S)-5-(5-(dimethylcarbamoyl)pyridin-2-yl)- |
| | | | | | N-(3-fluoro-4-(4-methylpiperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 607 | 0.002 | 2 | 0.021 | 2 | (S)-5-(5-(dimethylcarbamoyl)pyridin-2-yl)- |
| | | | | | N-(3-fluoro-4-(4-methyl-1,4-diazepane-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 608 | 0.001 | 2 | 0.016 | 2 | (S)-5-(5-(dimethylcarbamoyl)pyridin-2-yl)- |
| | | | | | N-(3-fluoro-4-(7-methyl-2,7- |
| | | | | | diazaspiro[3.5]nonane-2- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 609 | 0.001 | 2 | 0.007 | 2 | (S)-1-isopropyl-7-methyl-N-(4-(4- |
| | | | | | methylpiperazine-1-carboxamido)benzyl)-5- |
| | | | | | (2-oxo-2H-[1,3′-bipyridin]-6′-yl)-4,5,6,7- |
| | | | | | tetrahydro-1H-pyrazolo[4,3-c]pyridine-3- |
| | | | | | carboxamide |
| 610 | 0.002 | 2 | 0.017 | 2 | (S)-N-(3-fluoro-4-(4-methylpiperazine-1- |
| | | | | | carboxamido)benzyl)-1-isopropyl-7-methyl- |
| | | | | | 5-(2-oxo-2H-[1,3′-bipyridin]-6′-yl)-4,5,6,7- |
| | | | | | tetrahydro-1H-pyrazolo[4,3-c]pyridine-3- |
| | | | | | carboxamide |
| 611 | 0.0002 | 2 | 0.005 | 2 | (S)-5-(5-(6,7-dihydro-5H-pyrrolo[3,4- |
| | | | | | b]pyridine-6-carbonyl)pyridin-2-yl)-1- |
| | | | | | isopropyl-7-methyl-N-(4-(4- |
| | | | | | methylpiperazine-1-carboxamido)benzyl)- |
| | | | | | 4,5,6,7-tetrahydro-1H-pyrazolo[4,3- |
| | | | | | c]pyridine-3-carboxamide |
| 612 | 0.0005 | 2 | 0.010 | 2 | (S)-5-(5-(6,7-dihydro-5H-pyrrolo[3,4- |
| | | | | | b]pyridine-6-carbonyl)pyridin-2-yl)-N-(3- |
| | | | | | fluoro-4-(4-methylpiperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 613 | 0.003 | 1 | ND | ND | (S)-5-(5-(1,3-dimethyl-5-oxo-1,5-dihydro- |
| | | | | | 4H-1,2,4-triazol-4-yl)pyridin-2-yl)-1- |
| | | | | | isopropyl-7-methyl-N-(4-(4- |
| | | | | | methylpiperazine-1-carboxamido)benzyl)- |
| | | | | | 4,5,6,7-tetrahydro-1H-pyrazolo[4,3- |
| | | | | | c]pyridine-3-carboxamide |
| 614 | 0.0004 | 1 | 0.007 | 1 | (S)-5-(4-(6,7-dihydro-5H-pyrrolo[3,4- |
| | | | | | b]pyridine-6-carbonyl)phenyl)-1-isopropyl- |
| | | | | | 7-methyl-N-(4-(4-methylpiperazine-1- |
| | | | | | carboxamido)benzyl)-4,5,6,7-tetrahydro- |
| | | | | | 1H-pyrazolo[4,3-c]pyridine-3-carboxamide |
| 615 | 0.002 | 1 | 0.013 | 1 | (S)-5-(5-(dimethylcarbamoyl)pyridin-2-yl)-1- |
| | | | | | isopropyl-7-methyl-N-(4-(4-methyl-1,4- |
| | | | | | diazepane-1-carboxamido)benzyl)-4,5,6,7- |
| | | | | | tetrahydro-1H-pyrazolo[4,3-c]pyridine-3- |
| | | | | | carboxamide |
| 616 | 0.001 | 1 | 0.006 | 1 | (S)-N-(4-(4-(dimethylamino)piperidine-1- |
| | | | | | carboxamido)benzyl)-5-(5- |
| | | | | | (dimethylcarbamoyl)pyridin-2-yl)-1- |
| | | | | | isopropyl-7-methyl-4,5,6,7-tetrahydro-1H- |
| | | | | | pyrazolo[4,3-c]pyridine-3-carboxamide |
| 617 | 0.178 | 1 | 0.502 | 1 | (S)-5-(4-(dimethylcarbamoyl)phenyl)-1- |
| | | | | | isopropyl-7-methyl-N-(4-(((1- |
| | | | | | methylpiperidin-4- |
| | | | | | yl)methyl)carbamoyl)benzyl)-4,5,6,7- |
| | | | | | tetrahydro-1H-pyrazolo[4,3-c]pyridine-3- |
| | | | | | carboxamide |
| 618 | 0.127 | 1 | 0.612 | 1 | (S)-5-(5-(dimethylcarbamoyl)pyridin-2-yl)-1- |
| | | | | | isopropyl-7-methyl-N-(4-(((1- |
| | | | | | methylpiperidin-4- |
| | | | | | yl)methyl)carbamoyl)benzyl)-4,5,6,7- |
| | | | | | tetrahydro-1H-pyrazolo[4,3-c]pyridine-3- |
| | | | | | carboxamide |
| 619 | 0.003 | 1 | 0.019 | 1 | (S)-5-(4-(dimethylcarbamoyl)phenyl)-1- |
| | | | | | isopropyl-7-methyl-N-(4-(3- |
| | | | | | (methylamino)azetidine-1- |
| | | | | | carboxamido)benzyl)-4,5,6,7-tetrahydro- |
| | | | | | 1H-pyrazolo[4,3-c]pyridine-3-carboxamide |
| 620 | 0.001 | 1 | 0.008 | 1 | (S)-N-(4-([4,4′-bipiperidine]-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 621 | 0.007 | 1 | 0.031 | 1 | (S)-N-(4-(1,6-diazaspiro[3.3]heptane-6- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 622 | 0.004 | 1 | 0.016 | 1 | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)phenyl)-2,9- |
| | | | | | diazaspiro[5.5]undecane-9-carboxamide |
| 623 | 0.013 | 1 | 0.037 | 1 | (S)-N-(4-(2,7-diazaspiro[3.5]nonane-7- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 624 | 0.001 | 1 | 0.018 | 1 | (S)-5-(4-(dimethylcarbamoyl)phenyl)-1- |
| | | | | | isopropyl-7-methyl-N-(4-(4-(piperidin-4- |
| | | | | | ylmethyl)piperidine-1-carboxamido)benzyl)- |
| | | | | | 4,5,6,7-tetrahydro-1H-pyrazolo[4,3- |
| | | | | | c]pyridine-3-carboxamide |
| 625 | 0.010 | 1 | 0.024 | 1 | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)phenyl)-3,9- |
| | | | | | diazaspiro[5.5]undecane-3-carboxamide |
| 626 | 0.038 | 1 | 0.088 | 1 | (S)-N-(4-(2,7-diazaspiro[3.5]nonane-2- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 627 | 0.007 | 1 | 0.023 | 1 | (S)-N-(4-(3-aminoazetidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 628 | 0.001 | 1 | 0.004 | 1 | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)phenyl)-1-oxa-4,9- |
| | | | | | diazaspiro[5.5]undecane-9-carboxamide |
| 629 | 0.121 | 1 | 0.390 | 1 | (S)-N-(4-(3,3-dimethylureido)benzyl)-1- |
| | | | | | isopropyl-7-methyl-5-(4-(methyl((1- |
| | | | | | methylazetidin-3- |
| | | | | | yl)methyl)carbamoyl)phenyl)-4,5,6,7- |
| | | | | | tetrahydro-1H-pyrazolo[4,3-c]pyridine-3- |
| | | | | | carboxamide |
| 630 | 0.009 | 1 | 0.055 | 1 | (S)-N-(2-chloro-4-(piperazine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 631 | 0.001 | 1 | 0.024 | 1 | (S)-5-(5-(2H-1,2,3-triazol-2-yl)pyridin-2-yl)- |
| | | | | | 1-isopropyl-7-methyl-N-(4-(4- |
| | | | | | methylpiperazine-1-carboxamido)benzyl)- |
| | | | | | 4,5,6,7-tetrahydro-1H-pyrazolo[4,3- |
| | | | | | c]pyridine-3-carboxamide |
| 632 | 0.005 | 1 | 0.039 | 1 | (S)-N-(3-fluoro-4-(7-methyl-2,7- |
| | | | | | diazaspiro[3.5]nonane-2- |
| | | | | | carboxamido)benzyl)-1-isopropyl-7-methyl- |
| | | | | | 5-(2-oxo-2H-[1,3′-bipyridin]-6′-yl)-4,5,6,7- |
| | | | | | tetrahydro-1H-pyrazolo[4,3-c]pyridine-3- |
| | | | | | carboxamide |
| 633 | 0.002 | 1 | 0.013 | 1 | (S)-1-isopropyl-7-methyl-N-(4-(2-methyl- |
| | | | | | 2,7-diazaspiro[3.5]nonane-7- |
| | | | | | carboxamido)benzyl)-5-(2-oxo-2H-[1,3′- |
| | | | | | bipyridin]-6′-yl)-4,5,6,7-tetrahydro-1H- |
| | | | | | pyrazolo[4,3-c]pyridine-3-carboxamide |
| 634 | 0.004 | 1 | 0.029 | 1 | (S)-1-isopropyl-7-methyl-N-(4-(7-methyl- |
| | | | | | 2,7-diazaspiro[3.5]nonane-2- |
| | | | | | carboxamido)benzyl)-5-(2-oxo-2H-[1,3′- |
| | | | | | bipyridin]-6′-yl)-4,5,6,7-tetrahydro-1H- |
| | | | | | pyrazolo[4,3-c]pyridine-3-carboxamide |
| 635 | 0.014 | 1 | 0.071 | 1 | (S)-N-(4-(3,3-dimethylureido)benzyl)-1- |
| | | | | | isopropyl-7-methyl-5-(4-(methyl((1- |
| | | | | | methylpiperidin-4- |
| | | | | | yl)methyl)carbamoyl)phenyl)-4,5,6,7- |
| | | | | | tetrahydro-1H-pyrazolo[4,3-c]pyridine-3- |
| | | | | | carboxamide |
| 636 | 0.003 | 1 | 0.033 | 1 | (S)-N-(4-(3,3-dimethylureido)benzyl)-1- |
| | | | | | isopropyl-7-methyl-5-(4-(methyl(2- |
| | | | | | morpholinoethyl)carbamoyl)phenyl)- |
| | | | | | 4,5,6,7-tetrahydro-1H-pyrazolo[4,3- |
| | | | | | c]pyridine-3-carboxamide |
| 637 | 0.013 | 1 | 0.048 | 1 | (S)-N-(4-(3,3-dimethylureido)benzyl)-1- |
| | | | | | isopropyl-7-methyl-5-(4-(methyl(piperidin-4- |
| | | | | | ylmethyl)carbamoyl)phenyl)-4,5,6,7- |
| | | | | | tetrahydro-1H-pyrazolo[4,3-c]pyridine-3- |
| | | | | | carboxamide |
| 638 | 0.001 | 1 | 0.004 | 1 | (S)-N-(4-(4-amino-4- |
| | | | | | (methoxymethyl)piperidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 639 | 0.001 | 1 | 0.008 | 1 | (S)-N-(4-(4-amino-4- |
| | | | | | (methoxymethyl)piperidine-1-carboxamido)- |
| | | | | | 3-fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 640 | 0.001 | 1 | ND | ND | (S)-N-(4-(((S)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamido)methyl)-2- |
| | | | | | fluorophenyl)octahydro-2H-pyrido[1,2- |
| | | | | | a]lpyrazine-2-carboxamide |
| 641 | 0.005 | 1 | ND | ND | (S)-5-(4-(dimethylcarbamoyl)phenyl)-N-(3- |
| | | | | | fluoro-4-(6-methyl-2,6- |
| | | | | | diazaspiro[3.3]heptane-2- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 642 | 0.001 | 1 | ND | ND | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)-2-fluorophenyl)-2- |
| | | | | | methyl-2,8-diazaspiro[4.5]decane-8- |
| | | | | | carboxamide |
| 643 | 0.001 | 1 | ND | ND | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)-2-fluorophenyl)-4- |
| | | | | | methyl-1-oxa-4,9-diazaspiro[5.5]undecane- |
| | | | | | 9-carboxamide |
| 644 | 0.001 | 1 | ND | ND | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)-2-fluorophenyl)-2- |
| | | | | | methyl-2,9-diazaspiro[5.5]undecane-9- |
| | | | | | carboxamide |
| 645 | 0.001 | 1 | ND | ND | (S)-5-(4-(dimethylcarbamoyl)phenyl)-N-(3- |
| | | | | | fluoro-4-(1-methyl-1,7- |
| | | | | | diazaspiro[3.5]nonane-7- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 646 | 0.001 | 1 | ND | ND | (S)-5-(4-(dimethylcarbamoyl)phenyl)-N-(3- |
| | | | | | fluoro-4-(6-methyl-2,6- |
| | | | | | diazaspiro[3.5]nonane-2- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 647 | 0.001 | 1 | ND | ND | (S)-5-(4-(dimethylcarbamoyl)phenyl)-N-(3- |
| | | | | | fluoro-4-(2-methyl-2,7- |
| | | | | | diazaspiro[3.5]nonane-7- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 648 | 0.001 | 1 | ND | ND | (S)-5-(4-(dimethylcarbamoyl)phenyl)-N-(3- |
| | | | | | fluoro-4-(4-methyl-4,7- |
| | | | | | diazaspiro[2.5]octane-7- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 649 | 0.001 | 1 | 0.024 | 1 | (S)-5-(5-(1-cyclopropyl-1H-1,2,4-triazol-5- |
| | | | | | yl)pyridin-2-yl)-1-isopropyl-7-methyl-N-(4- |
| | | | | | (4-methylpiperazine-1- |
| | | | | | carboxamido)benzyl)-4,5,6,7-tetrahydro- |
| | | | | | 1H-pyrazolo[4,3-c]pyridine-3-carboxamide |
| 651 | 0.001 | 1 | ND | ND | (S)-1-isopropyl-7-methyl-5-(2-methyl-1-oxo- |
| | | | | | 2,3,4,5-tetrahydro-1H-benzo[c]azepin-7-yl)- |
| | | | | | N-(4-(4-methylpiperazine-1- |
| | | | | | carboxamido)benzyl)-4,5,6,7-tetrahydro- |
| | | | | | 1H-pyrazolo[4,3-c]pyridine-3-carboxamide |
| 652 | 0.005 | 1 | ND | ND | (S)-5-(4-(1,3-dimethyl-5-oxo-1,5-dihydro- |
| | | | | | 4H-1,2,4-triazol-4-yl)phenyl)-1-isopropyl-7- |
| | | | | | methyl-N-(4-(4-methylpiperazine-1- |
| | | | | | carboxamido)benzyl)-4,5,6,7-tetrahydro- |
| | | | | | 1H-pyrazolo[4,3-c]pyridine-3-carboxamide |
| 653 | 0.003 | 1 | ND | ND | (S)-5-(5-(1-cyclopropyl-1H-imidazol-2- |
| | | | | | yl)pyridin-2-yl)-1-isopropyl-7-methyl-N-(4- |
| | | | | | (4-methylpiperazine-1- |
| | | | | | carboxamido)benzyl)-4,5,6,7-tetrahydro- |
| | | | | | 1H-pyrazolo[4,3-c]pyridine-3-carboxamide |
| 654 | 0.002 | 2 | ND | ND | (S)-5-(5-(1-(difluoromethyl)-1H-imidazol-2- |
| | | | | | yl)pyridin-2-yl)-1-isopropyl-7-methyl-N-(4- |
| | | | | | (4-methylpiperazine-1- |
| | | | | | carboxamido)benzyl)-4,5,6,7-tetrahydro- |
| | | | | | 1H-pyrazolo[4,3-c]pyridine-3-carboxamide |
| 655 | 0.002 | 2 | ND | ND | (S)-5-(5-(4-cyclopropyl-4H-1,2,4-triazol-3- |
| | | | | | yl)pyridin-2-yl)-1-isopropyl-7-methyl-N-(4- |
| | | | | | (4-methylpiperazine-1- |
| | | | | | carboxamido)benzyl)-4,5,6,7-tetrahydro- |
| | | | | | 1H-pyrazolo[4,3-c]pyridine-3-carboxamide |
| 656 | 0.002 | 1 | ND | ND | (S)-1-isopropyl-7-methyl-5-(4-(3-methyl-5- |
| | | | | | oxo-1,2,4-oxadiazol-4(5H)-yl)phenyl)-N-(4- |
| | | | | | (4-methylpiperazine-1- |
| | | | | | carboxamido)benzyl)-4,5,6,7-tetrahydro- |
| | | | | | 1H-pyrazolo[4,3-c]pyridine-3-carboxamide |
| 657 | 0.002 | 1 | ND | ND | (S)-1-isopropyl-7-methyl-5-(4-methyl-5-oxo- |
| | | | | | 2,3,4,5-tetrahydrobenzo[f][1,4]oxazepin-8- |
| | | | | | yl)-N-(4-(4-methylpiperazine-1- |
| | | | | | carboxamido)benzyl)-4,5,6,7-tetrahydro- |
| | | | | | 1H-pyrazolo[4,3-c]pyridine-3-carboxamide |
| 658 | 0.004 | 1 | ND | ND | (S)-1-isopropyl-7-methyl-5-(5-(4-methyl-4H- |
| | | | | | 1,2,4-triazol-3-yl)pyridin-2-yl)-N-(4-(4- |
| | | | | | methylpiperazine-1-carboxamido)benzyl)- |
| | | | | | 4,5,6,7-tetrahydro-1H-pyrazolo[4,3- |
| | | | | | c]pyridine-3-carboxamide |
| 659 | 0.008 | 1 | ND | ND | (S)-N-(4-(3-(dimethylamino)-3- |
| | | | | | methylazetidine-1-carboxamido)-3- |
| | | | | | fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 660 | 0.004 | 1 | ND | ND | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)-2-fluorophenyl)-9- |
| | | | | | methyl-3,9-diazaspiro[5.5]undecane-3- |
| | | | | | carboxamide |
| 661 | 0.005 | 1 | ND | ND | (S)-N-(4-(3,3-dimethylureido)benzyl)-1- |
| | | | | | isopropyl-7-methyl-5-(4- |
| | | | | | (methyl(((1R,5S,6r)-3-methyl-3- |
| | | | | | azabicyclo[3.1.0]hexan-6- |
| | | | | | yl)methyl)carbamoyl)phenyl)-4,5,6,7- |
| | | | | | tetrahydro-1H-pyrazolo[4,3-c]pyridine-3- |
| | | | | | carboxamide |
| 662 | 0.003 | 1 | ND | ND | (S)-5-(4-(((1-benzylpiperidin-4- |
| | | | | | yl)methyl)(methyl)carbamoyl)phenyl)-N-(4- |
| | | | | | (3,3-dimethylureido)benzyl)-1-isopropyl-7- |
| | | | | | methyl-4,5,6,7-tetrahydro-1H-pyrazolo[4,3- |
| | | | | | c]pyridine-3-carboxamide |
| 669 | 0.201 | 1 | ND | ND | (S)-3-(4-((4-((5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamido)methyl)-2- |
| | | | | | fluorophenyl)carbamoyl)piperazin-1- |
| | | | | | yl)propanoic acid |
| 670\hat{ } | 0.001 | 1 | ND | ND | (S)-N-(4-((R*)-3-amino-3-(2- |
| | | | | | methoxyethyl)pyrrolidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 671\hat{ } | 0.003 | 1 | ND | ND | (S)-N-(4-((R*)-3-amino-3-(2- |
| | | | | | methoxyethyl)pyrrolidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 672\hat{ } | 0.001 | 1 | ND | ND | (S)-N-(4-((R*)-3-amino-3- |
| | | | | | (methoxymethyl)pyrrolidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 673\hat{ } | 0.002 | 1 | ND | ND | (S)-N-(4-((R*)-3-amino-3- |
| | | | | | (methoxymethyl)pyrrolidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 674 | 0.0002 | 1 | ND | ND | (S)-5-(4-(1H-1,2,3-triazol-1-yl)phenyl)-1- |
| | | | | | isopropyl-7-methyl-N-(4-(4- |
| | | | | | methylpiperazine-1-carboxamido)benzyl)- |
| | | | | | 4,5,6,7-tetrahydro-1H-pyrazolo[4,3- |
| | | | | | c]pyridine-3-carboxamide |
| 675 | 0.034 | 1 | ND | ND | (S)-N-(4-(3,3-dimethylureido)benzyl)-1- |
| | | | | | isopropyl-7-methyl-5-(4-((R)- |
| | | | | | octahydropyrrolo[1,2-a]pyrazine-2- |
| | | | | | carbonyl)phenyl)-4,5,6,7-tetrahydro-1H- |
| | | | | | pyrazolo[4,3-c]pyridine-3-carboxamide |
| 676 | 0.0003 | 1 | ND | ND | (S)-5-(4-(6,7-dihydro-5H-pyrrolo[3,4- |
| | | | | | b]pyridine-6-carbonyl)phenyl)-N-(4-(3,3- |
| | | | | | dimethylureido)benzyl)-1-isopropyl-7- |
| | | | | | methyl-4,5,6,7-tetrahydro-1H-pyrazolo[4,3- |
| | | | | | c]pyridine-3-carboxamide |
| 678 | 0.003 | 1 | ND | ND | (S)-N-(4-((R)-3-(dimethylamino)pyrrolidine- |
| | | | | | 1-carboxamido)-3-fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 679 | 0.003 | 1 | ND | ND | (7S)-N-(4-(3-(dimethylamino)-3- |
| | | | | | methylpyrrolidine-1-carboxamido)-3- |
| | | | | | fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 680 | 0.002 | 1 | ND | ND | (S)-5-(4-(dimethylcarbamoyl)phenyl)-N-(4- |
| | | | | | ((S)-3,4-dimethylpiperazine-1- |
| | | | | | carboxamido)-3-fluorobenzyl)-1-isopropyl- |
| | | | | | 7-methyl-4,5,6,7-tetrahydro-1H- |
| | | | | | pyrazolo[4,3-c]pyridine-3-carboxamide |
| 681 | 0.003 | 1 | ND | ND | (S)-N-(4-((S)-3- |
| | | | | | ((dimethylamino)methyl)pyrrolidine-1- |
| | | | | | carboxamido)-3-fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 682 | 0.004 | 1 | ND | ND | (S)-N-(4-((R)-3-(dimethylamino)piperidine- |
| | | | | | 1-carboxamido)-3-fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 683 | 0.003 | 1 | ND | ND | (S)-N-(4-((S)-3-(dimethylamino)piperidine- |
| | | | | | 1-carboxamido)-3-fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 684 | 0.002 | 1 | ND | ND | (S)-N-(4-((R)-3- |
| | | | | | ((dimethylamino)methyl)pyrrolidine-1- |
| | | | | | carboxamido)-3-fluorobenzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 685 | 0.002 | 1 | ND | ND | (S)-5-(4-(dimethylcarbamoyl)phenyl)-N-(3- |
| | | | | | fluoro-4-(3-(3-methylazetidin-3- |
| | | | | | yl)ureido)benzyl)-1-isopropyl-7-methyl- |
| | | | | | 4,5,6,7-tetrahydro-1H-pyrazolo[4,3- |
| | | | | | c]pyridine-3-carboxamide |
| 686 | 0.001 | 1 | ND | ND | (S)-5-(4-(dimethylcarbamoyl)phenyl)-N-(4- |
| | | | | | ((R)-3,4-dimethylpiperazine-1- |
| | | | | | carboxamido)-3-fluorobenzyl)-1-isopropyl- |
| | | | | | 7-methyl-4,5,6,7-tetrahydro-1H- |
| | | | | | pyrazolo[4,3-c]pyridine-3-carboxamide |
| 687 | 0.001 | 1 | ND | ND | (R)-N-(4-(((S)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamido)methyl)-2- |
| | | | | | fluorophenyl)octahydro-2H-pyrido[1,2- |
| | | | | | a]lpyrazine-2-carboxamide |
| 688 | 0.001 | 1 | ND | ND | (S)-5-(5-(dimethylcarbamoyl)pyridin-2-yl)- |
| | | | | | N-(3-fluoro-4-((R)-3- |
| | | | | | (methoxymethyl)piperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 689 | 0.001 | 1 | ND | ND | (S)-5-(4-(dimethylcarbamoyl)phenyl)-N-(3- |
| | | | | | fluoro-4-((R)-3-(methoxymethyl)piperazine- |
| | | | | | 1-carboxamido)benzyl)-1-isopropyl-7- |
| | | | | | methyl-4,5,6,7-tetrahydro-1H-pyrazolo[4,3- |
| | | | | | c]pyridine-3-carboxamide |
| 690 | 0.001 | 1 | ND | ND | (S)-5-(5-(dimethylcarbamoyl)pyridin-2-yl)- |
| | | | | | N-(3-fluoro-4-((R)-octahydropyrrolo[1,2- |
| | | | | | a]lpyrazine-2-carboxamido)benzyl)-1- |
| | | | | | isopropyl-7-methyl-4,5,6,7-tetrahydro-1H- |
| | | | | | pyrazolo[4,3-c]pyridine-3-carboxamide |
| 691 | 0.001 | 1 | ND | ND | (S)-5-(5-(dimethylcarbamoyl)pyridin-2-yl)-1- |
| | | | | | isopropyl-7-methyl-N-(4-((R)- |
| | | | | | octahydropyrrolo[1,2-a]pyrazine-2- |
| | | | | | carboxamido)benzyl)-4,5,6,7-tetrahydro- |
| | | | | | 1H-pyrazolo[4,3-c]pyridine-3-carboxamide |
| 692 | 0.0003 | 1 | ND | ND | rel-(R or S)-5-(5- |
| | | | | | (dimethylcarbamoyl)pyridin-2-yl)-1- |
| | | | | | isopropyl-N-(4-(4-methylpiperazine-1- |
| | | | | | carboxamido)benzyl)-7-(trifluoromethyl)- |
| | | | | | 4,5,6,7-tetrahydro-1H-pyrazolo[4,3- |
| | | | | | c]pyridine-3-carboxamide |
| 693 | 0.016 | 1 | ND | ND | rel-(R or S)-5-(5- |
| | | | | | (dimethylcarbamoyl)pyridin-2-yl)-1- |
| | | | | | isopropyl-N-(4-(4-methylpiperazine-1- |
| | | | | | carboxamido)benzyl)-7-(trifluoromethyl)- |
| | | | | | 4,5,6,7-tetrahydro-1H-pyrazolo[4,3- |
| | | | | | c]pyridine-3-carboxamide |
| 694 | 0.0003 | 1 | ND | ND | rel-(R or S)-5-(5- |
| | | | | | (dimethylcarbamoyl)pyridin-2-yl)-1-ethyl-N- |
| | | | | | (4-(4-methylpiperazine-1- |
| | | | | | carboxamido)benzyl)-7-(trifluoromethyl)- |
| | | | | | 4,5,6,7-tetrahydro-1H-pyrazolo[4,3- |
| | | | | | c]pyridine-3-carboxamide |
| 695 | 0.032 | 1 | ND | ND | rel-(R or S)-5-(5- |
| | | | | | (dimethylcarbamoyl)pyridin-2-yl)-1-ethyl-N- |
| | | | | | (4-(4-methylpiperazine-1- |
| | | | | | carboxamido)benzyl)-7-(trifluoromethyl)- |
| | | | | | 4,5,6,7-tetrahydro-1H-pyrazolo[4,3- |
| | | | | | c]pyridine-3-carboxamide |
| 699 | 0.004 | 1 | 0.027 | 1 | (S)-5-(4-(dimethylcarbamoyl)phenyl)-1- |
| | | | | | isopropyl-7-methyl-N-((6-(piperazine-1- |
| | | | | | carboxamido)pyridin-3-yl)methyl)-4,5,6,7- |
| | | | | | tetrahydro-1H-pyrazolo[4,3-c]pyridine-3- |
| | | | | | carboxamide |
| 700 | 0.005 | 1 | 0.022 | 1 | (S)-5-(4-(dimethylcarbamoyl)phenyl)-1- |
| | | | | | isopropyl-7-methyl-N-((5-(piperazine-1- |
| | | | | | carboxamido)pyridin-2-yl)methyl)-4,5,6,7- |
| | | | | | tetrahydro-1H-pyrazolo[4,3-c]pyridine-3- |
| | | | | | carboxamide |
| 701 | 0.0004 | 1 | ND | ND | rac-(R)-5-(5-(dimethylcarbamoyl)pyridin-2- |
| | | | | | yl)-1-isopropyl-N-(4-(4-methylpiperazine-1- |
| | | | | | carboxamido)benzyl)-7-(trifluoromethyl)- |
| | | | | | 4,5,6,7-tetrahydro-1H-pyrazolo[4,3- |
| | | | | | c]pyridine-3-carboxamide |
| 702 | 0.010 | 1 | ND | ND | rel-(R or S)-5-(4- |
| | | | | | (dimethylcarbamoyl)phenyl)-1-isopropyl-N- |
| | | | | | (4-(4-methylpiperazine-1- |
| | | | | | carboxamido)benzyl)-7-(trifluoromethyl)- |
| | | | | | 4,5,6,7-tetrahydro-1H-pyrazolo[4,3- |
| | | | | | c]pyridine-3-carboxamide |
| 703 | 0.001 | 1 | ND | ND | rac-(R)-5-(4-(dimethylcarbamoyl)phenyl)-1- |
| | | | | | isopropyl-N-(4-(4-methylpiperazine-1- |
| | | | | | carboxamido)benzyl)-7-(trifluoromethyl)- |
| | | | | | 4,5,6,7-tetrahydro-1H-pyrazolo[4,3- |
| | | | | | c]pyridine-3-carboxamide |
| 704 | 0.001 | 1 | ND | ND | rac-(R)-5-(4-(dimethylcarbamoyl)phenyl)-1- |
| | | | | | isopropyl-N-(4-(piperazine-1- |
| | | | | | carboxamido)benzyl)-7-(trifluoromethyl)- |
| | | | | | 4,5,6,7-tetrahydro-1H-pyrazolo[4,3- |
| | | | | | c]pyridine-3-carboxamide |
| 705 | 0.0002 | 1 | ND | ND | rel-(R or S)-5-(4- |
| | | | | | (dimethylcarbamoyl)phenyl)-1-isopropyl-N- |
| | | | | | (4-(4-methylpiperazine-1- |
| | | | | | carboxamido)benzyl)-7-(trifluoromethyl)- |
| | | | | | 4,5,6,7-tetrahydro-1H-pyrazolo[4,3- |
| | | | | | c]pyridine-3-carboxamide |
| 706 | 0.027 | 1 | 0.191 | 1 | (S)-N-(4-(3,3-dimethylureido)benzyl)-1- |
| | | | | | isopropyl-7-methyl-5-(4-(4- |
| | | | | | methylpiperazine-1-carbonyl)phenyl)- |
| | | | | | 4,5,6,7-tetrahydro-1H-pyrazolo[4,3- |
| | | | | | c]pyridine-3-carboxamide |
| 708 | 0.001 | 1 | 0.008 | 1 | (S)-N-(4-(4-amino-4-(2- |
| | | | | | methoxyethyl)piperidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 711 | 0.0005 | 1 | 0.018 | 1 | (S)-5-(4-(6,7-dihydro-5H-pyrrolo[3,4- |
| | | | | | b]pyridine-6-carbonyl)phenyl)-N-(3-fluoro-4- |
| | | | | | (4-methylpiperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 712 | 0.003 | 1 | 0.064 | 1 | (S)-N-(2-chloro-4-(4-methylpiperazine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 713 | 0.003 | 1 | 0.022 | 1 | (S)-5-(4-(dimethylcarbamoyl)phenyl)-1- |
| | | | | | isopropyl-7-methyl-N-(4-((R*)-3-methyl-3- |
| | | | | | (methylamino)pyrrolidine-1- |
| | | | | | carboxamido)benzyl)-4,5,6,7-tetrahydro- |
| | | | | | 1H-pyrazolo[4,3-c]pyridine-3-carboxamide |
| 714 | 0.004 | 1 | 0.028 | 1 | (S)-5-(4-(dimethylcarbamoyl)phenyl)-1- |
| | | | | | isopropyl-7-methyl-N-(4-((R*)-3-methyl-3- |
| | | | | | (methylamino)pyrrolidine-1- |
| | | | | | carboxamido)benzyl)-4,5,6,7-tetrahydro- |
| | | | | | 1H-pyrazolo[4,3-c]pyridine-3-carboxamide |
| 715 | 0.005 | 2 | 0.068 | 2 | (S)-N-(3-fluoro-4-(4-methylpiperazine-1- |
| | | | | | carboxamido)benzyl)-1-isopropyl-7-methyl- |
| | | | | | 5-(4-(2-oxopyridin-1(2H)-yl)phenyl)-4,5,6,7- |
| | | | | | tetrahydro-1H-pyrazolo[4,3-c]pyridine-3- |
| | | | | | carboxamide |
| 716 | 0.003 | 2 | 0.025 | 2 | (S)-5-(4-(dimethylcarbamoyl)phenyl)-1- |
| | | | | | ethyl-N-(3-fluoro-4-(3- |
| | | | | | (methylamino)azetidine-1- |
| | | | | | carboxamido)benzyl)-7-methyl-4,5,6,7- |
| | | | | | tetrahydro-1H-pyrazolo[4,3-c]pyridine-3- |
| | | | | | carboxamide |
| 717 | 0.003 | 2 | 0.029 | 2 | (S)-N-(3-chloro-4-(4-methylpiperazine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 718 | 0.001 | 2 | 0.018 | 2 | (S)-5-(4-(dimethylcarbamoyl)phenyl)-N-(2- |
| | | | | | fluoro-4-(4-methylpiperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 719 | 1.002 | 2 | 3.690 | 2 | (S)-N-(4-carbamoylbenzyl)-1-isopropyl-7- |
| | | | | | methyl-5-(5-(1-methyl-1H-imidazol-2- |
| | | | | | yl)pyrimidin-2-yl)-4,5,6,7-tetrahydro-1H- |
| | | | | | pyrazolo[4,3-c]pyridine-3-carboxamide |
| 721 | 0.012 | 2 | 0.102 | 2 | (S)-5-(4-(dimethylcarbamoyl)phenyl)-N-(3- |
| | | | | | fluoro-4-(1-methylpiperidine-4- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 722 | 0.008 | 2 | 0.040 | 2 | (S)-5-(4-(dimethylcarbamoyl)phenyl)-1- |
| | | | | | isopropyl-7-methyl-N-(4-((R)-3- |
| | | | | | (methylamino)pyrrolidine-1- |
| | | | | | carboxamido)benzyl)-4,5,6,7-tetrahydro- |
| | | | | | 1H-pyrazolo[4,3-c]pyridine-3-carboxamide |
| 723 | 0.128 | 2 | 0.665 | 2 | (S)-5-(4-(dimethylcarbamoyl)phenyl)-1- |
| | | | | | isopropyl-7-methyl-N-(4-(4-methyloxazol-2- |
| | | | | | yl)benzyl)-4,5,6,7-tetrahydro-1H- |
| | | | | | pyrazolo[4,3-c]pyridine-3-carboxamide |
| 724 | 0.129 | 2 | 5.061 | 2 | (R)-5-(4-(dimethylcarbamoyl)phenyl)-1- |
| | | | | | isopropyl-7-methyl-N-(4-(6-methylpyrazin- |
| | | | | | 2-yl)benzyl)-4,5,6,7-tetrahydro-1H- |
| | | | | | pyrazolo[4,3-c]pyridine-3-carboxamide |
| 725 | 0.177 | 2 | 4.458 | 2 | (S)-5-(4-(dimethylcarbamoyl)phenyl)-1- |
| | | | | | isopropyl-7-methyl-N-(4-(1-methyl-1H- |
| | | | | | pyrazol-3-yl)benzyl)-4,5,6,7-tetrahydro-1H- |
| | | | | | pyrazolo[4,3-c]pyridine-3-carboxamide |
| 726 | 0.123 | 3 | 5.676 | 3 | (S)-5-(4-(dimethylcarbamoyl)phenyl)-1- |
| | | | | | isopropyl-7-methyl-N-(4-(6-methylpyridin-2- |
| | | | | | yl)benzyl)-4,5,6,7-tetrahydro-1H- |
| | | | | | pyrazolo[4,3-c]pyridine-3-carboxamide |
| 727 | 0.011 | 2 | 0.383 | 2 | (S)-5-(4-(dimethylcarbamoyl)phenyl)-1- |
| | | | | | isopropyl-7-methyl-N-(4-(6-methylpyridazin- |
| | | | | | 3-yl)benzyl)-4,5,6,7-tetrahydro-1H- |
| | | | | | pyrazolo[4,3-c]pyridine-3-carboxamide |
| 728 | 0.231 | 2 | 4.213 | 2 | (S)-5-(4-(dimethylcarbamoyl)phenyl)-1- |
| | | | | | isopropyl-7-methyl-N-(4-(2-methylpyrimidin- |
| | | | | | 4-yl)benzyl)-4,5,6,7-tetrahydro-1H- |
| | | | | | pyrazolo[4,3-c]pyridine-3-carboxamide |
| 729 | 0.683 | 2 | 4.327 | 2 | (S)-5-(4-(dimethylcarbamoyl)phenyl)-1- |
| | | | | | isopropyl-7-methyl-N-(4-(4-methylthiazol-2- |
| | | | | | yl)benzyl)-4,5,6,7-tetrahydro-1H- |
| | | | | | pyrazolo[4,3-c]pyridine-3-carboxamide |
| 730 | >10 | 1 | >15.12 | 2 | (S)-5-(4-(dimethylcarbamoyl)phenyl)-1- |
| | | | | | isopropyl-7-methyl-N-(4-(4-methylpyrimidin- |
| | | | | | 2-yl)benzyl)-4,5,6,7-tetrahydro-1H- |
| | | | | | pyrazolo[4,3-c]pyridine-3-carboxamide |
| 731 | 0.082 | 2 | 1.246 | 2 | (S)-N-(4-(5-amino-4-methoxypyrimidin-2- |
| | | | | | yl)benzyl)-5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3-carboxamide |
| 732 | 0.088 | 2 | 3.073 | 2 | (S)-5-(4-(dimethylcarbamoyl)phenyl)-1- |
| | | | | | isopropyl-7-methyl-N-(4-(1-methyl-1H- |
| | | | | | pyrazol-4-yl)benzyl)-4,5,6,7-tetrahydro-1H- |
| | | | | | pyrazolo[4,3-c]pyridine-3-carboxamide |
| 733 | 0.342 | 1 | ND | ND | (S)-3-(3-(4-((5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamido)methyl)-2- |
| | | | | | fluorophenyl)-1-methylureido)propanoic |
| | | | | | acid |
| 734 | 0.038 | 1 | ND | ND | (S)-1-((4-((5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamido)methyl)-2- |
| | | | | | fluorophenyl)carbamoyl)piperidine-4- |
| | | | | | carboxylic acid |
| 735 | 0.001 | 1 | ND | ND | (S)-5-(4-(dimethylcarbamoyl)phenyl)-N-(4- |
| | | | | | ((S)-3,4-dimethylpiperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 736 | 0.001 | 1 | ND | ND | (S)-N-(4-((S)-3- |
| | | | | | ((dimethylamino)methyl)pyrrolidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 737 | 0.001 | 1 | ND | ND | (S)-N-(4-(((S)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3- |
| | | | | | carboxamido)methyl)phenyl)octahydro-2H- |
| | | | | | pyrido[1,2-a]pyrazine-2-carboxamide |
| 738 | 0.001 | 1 | ND | ND | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)phenyl)-4-methyl-1- |
| | | | | | oxa-4,9-diazaspiro[5.5]undecane-9- |
| | | | | | carboxamide |
| 739 | 0.002 | 1 | ND | ND | (S)-5-(4-(dimethylcarbamoyl)phenyl)-1- |
| | | | | | isopropyl-7-methyl-N-(4-(3-(3- |
| | | | | | methylazetidin-3-yl)ureido)benzyl)-4,5,6,7- |
| | | | | | tetrahydro-1H-pyrazolo[4,3-c]pyridine-3- |
| | | | | | carboxamide |
| 740 | 0.002 | 1 | ND | ND | (S)-5-(4-(dimethylcarbamoyl)phenyl)-1- |
| | | | | | isopropyl-7-methyl-N-(4-(1-methyl-1,7- |
| | | | | | diazaspiro[3.5]nonane-7- |
| | | | | | carboxamido)benzyl)-4,5,6,7-tetrahydro- |
| | | | | | 1H-pyrazolo[4,3-c]pyridine-3-carboxamide |
| 741 | 0.001 | 1 | ND | ND | (S)-5-(4-(dimethylcarbamoyl)phenyl)-1- |
| | | | | | isopropyl-7-methyl-N-(4-(6-methyl-2,6- |
| | | | | | diazaspiro[3.5]nonane-2- |
| | | | | | carboxamido)benzyl)-4,5,6,7-tetrahydro- |
| | | | | | 1H-pyrazolo[4,3-c]pyridine-3-carboxamide |
| 742 | 0.001 | 1 | ND | ND | (S)-5-(4-(dimethylcarbamoyl)phenyl)-1- |
| | | | | | isopropyl-7-methyl-N-(4-(2-methyl-2,7- |
| | | | | | diazaspiro[3.5]nonane-7- |
| | | | | | carboxamido)benzyl)-4,5,6,7-tetrahydro- |
| | | | | | 1H-pyrazolo[4,3-c]pyridine-3-carboxamide |
| 743 | 0.001 | 1 | ND | ND | (S)-5-(4-(dimethylcarbamoyl)phenyl)-N-(4- |
| | | | | | ((R)-3,4-dimethylpiperazine-1- |
| | | | | | carboxamido)benzyl)-1-isopropyl-7-methyl- |
| | | | | | 4,5,6,7-tetrahydro-1H-pyrazolo[4,3- |
| | | | | | c]pyridine-3-carboxamide |
| 745 | 0.001 | 1 | ND | ND | (S)-5-(4-(dimethylcarbamoyl)phenyl)-N-(4- |
| | | | | | (4-ethylpiperazine-1-carboxamido)benzyl)- |
| | | | | | 1-isopropyl-7-methyl-4,5,6,7-tetrahydro-1H- |
| | | | | | pyrazolo[4,3-c]pyridine-3-carboxamide |
| 746 | 0.001 | 1 | ND | ND | (S)-N-(4-(3-(dimethylamino)azetidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 747 | 0.001 | 1 | ND | ND | (S)-N-(4-((S)-3-(dimethylamino)pyrrolidine- |
| | | | | | 1-carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 748 | 0.001 | 1 | ND | ND | (S)-N-(4-(3-(dimethylamino)-3- |
| | | | | | methylazetidine-1-carboxamido)benzyl)-5- |
| | | | | | (4-(dimethylcarbamoyl)phenyl)-1-isopropyl- |
| | | | | | 7-methyl-4,5,6,7-tetrahydro-1H- |
| | | | | | pyrazolo[4,3-c]pyridine-3-carboxamide |
| 749 | 0.0004 | 1 | ND | ND | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)phenyl)-9-methyl-3,9- |
| | | | | | diazaspiro[5.5]undecane-3-carboxamide |
| 750 | 0.001 | 1 | ND | ND | (S)-N-(4-((R)-3-(dimethylamino)pyrrolidine- |
| | | | | | 1-carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 751 | 0.009 | 1 | ND | ND | (S)-5-(4-(dimethylcarbamoyl)phenyl)-1- |
| | | | | | isopropyl-7-methyl-N-(4-(6-methyl-2,6- |
| | | | | | diazaspiro[3.3]heptane-2- |
| | | | | | carboxamido)benzyl)-4,5,6,7-tetrahydro- |
| | | | | | 1H-pyrazolo[4,3-c]pyridine-3-carboxamide |
| 752 | 0.002 | 1 | ND | ND | (7S)-N-(4-(3-(dimethylamino)-3- |
| | | | | | methylpyrrolidine-1-carboxamido)benzyl)-5- |
| | | | | | (4-(dimethylcarbamoyl)phenyl)-1-isopropyl- |
| | | | | | 7-methyl-4,5,6,7-tetrahydro-1H- |
| | | | | | pyrazolo[4,3-c]pyridine-3-carboxamide |
| 753 | 0.0005 | 1 | ND | ND | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)phenyl)-2-methyl-2,8- |
| | | | | | diazaspiro[4.5]decane-8-carboxamide |
| 754 | 0.002 | 1 | ND | ND | (S)-N-(4-((R)-3-(dimethylamino)piperidine- |
| | | | | | 1-carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 755 | 0.002 | 1 | ND | ND | (S)-N-(4-((S)-3-(dimethylamino)piperidine- |
| | | | | | 1-carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 756 | 0.001 | 1 | ND | ND | (S)-N-(4-((R)-3- |
| | | | | | ((dimethylamino)methyl)pyrrolidine-1- |
| | | | | | carboxamido)benzyl)-5-(4- |
| | | | | | (dimethylcarbamoyl)phenyl)-1-isopropyl-7- |
| | | | | | methyl-4,5,6,7-tetrahydro-1H-pyrazolo[4,3- |
| | | | | | c]pyridine-3-carboxamide |
| 757 | 0.0003 | 1 | ND | ND | (S)-N-(4-((5-(4-(dimethylcarbamoyl)phenyl)- |
| | | | | | 1-isopropyl-7-methyl-4,5,6,7-tetrahydro-1H- |
| | | | | | pyrazolo[4,3-c]pyridine-3- |
| | | | | | carboxamido)methyl)phenyl)-2-methyl-2,9- |
| | | | | | diazaspiro[5.5]undecane-9-carboxamide |

### Prophetic Deuterated Analogs

General methods/reviews of obtaining metabolite profile and identifying metabolites of a compound are described in: Dalvie, et al., “Assessment of Three Human in Vitro Systems in the Generation of Major Human Excretory and Circulating Metabolites,” Chemical Research in Toxicology, 2009, 22, 2, 357-368, tx8004357 (acs.orq); King, R., “Biotransformations in Drug Metabolism,” Ch.3, Drug Metabolism Handbook Introduction, https://doi.orq/10.1002/9781119851042.ch3; Wu, Y., et al, “Metabolite Identification in the Preclinical and Clinical Phase of Drug Development,” Current Drug Metabolism, 2021, 22, 11, 838-857, 10.2174/1389200222666211006104502; Godzien, J., et al, “Chapter Fifteen—Metabolite Annotation and Identification”.

Numerous publicly available and commercially available software tools are available to aid in the predictions of metabolic pathways and metabolites of compounds. Examples of such tools include, BioTransofrmer 3.0 (biotransformer.ca/new), which predicts the metabolic biotransformations of small molecules using a database of known metabolic reactions; MetaSite (moldiscovery.com/software/metasite/), which predicts metabolic transformations related to cytochrome P450 and flavin-containing monooxygenase mediated reactions in phase I metabolism; and Lhasa Meteor Nexus (lhasalimited.org/products/meteor-nexus.htm) offers prediction of metabolic pathways and metabolite structures using a range of machine learning models, which covers phase I and phase II biotransformations of small molecules.

Predicted deuterated analogs in the examples may afford certain therapeutic advantages resulting from greater metabolic stability, for example, increased in vivo half-life, reduced dosage requirements, increased STAT6 inhibition (competitive or time dependent), or an improvement in therapeutic index or tolerability.

A person with ordinary skill may make additional deuterated analogs of the disclosed compounds with different combinations of deuterium replacing hydrogen. Such additional deuterated analogs may provide similar therapeutic advantages that may be achieved by the deuterated analogs as illustrated below.

In the examples, the “Yᵢ” is used to indicate the order of the sites most likely to be metabolized based on MetaSite predictions for the respective compound, where i=1 indicates the most likely, i=2 indicates the second most likely site and so on. It is noted that where Yᵢ is indicated as being deuterated, D, each Yᵢ independently can be D as long as at least one Yᵢ is D. For example, if Y₁ is deuterated and there are two Y₁ groups in the compound, then one or both groups can be deuterated as long as at least one Y₁ group is deuterated.

These examples are not intended to be limited to the specific compounds or locations of deuteration.

### Example D1

Formula D1 is the generic formula of deuterated Example 1, wherein Y₁, Y₂, Y₃, Y₄, Y₅, Y₆, Y₇, Y₈, Y₉, and Y₁₀ are each independently H or D. Table 4 shows some examples of deuterated compounds based on the most likely metabolized sites that can be applied to the compounds predicted by MetaSite.

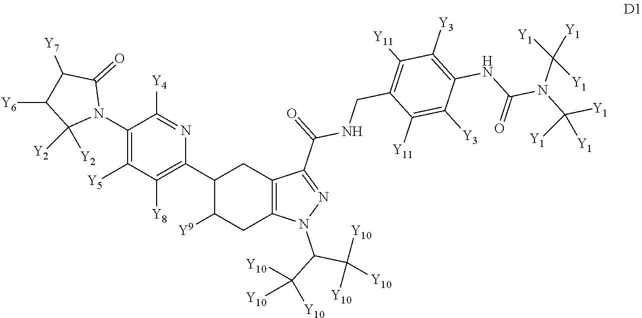

TABLE 4

| Deuteration | | | Y₃ and/ | | | | | | | |
|---|---|---|---|---|---|---|---|---|---|---|
| Example | Y₁ | Y₂ | or Y₁₁ | Y₄ | Y₅ | Y₆ | Y₇ | Y₈ | Y₉ | Y₁₀ |
| D1A | D | H | H | H | H | H | H | H | H | H |
| D1B | D | D | H | H | H | H | H | H | H | H |
| D1C | D | D | D | H | H | H | H | H | H | H |
| D1D | D | H | D | H | H | H | H | H | H | H |
| D1E | D | H | H | D | H | H | H | H | H | H |
| D1F | D | H | H | H | D | H | H | H | H | H |
| D1G | D | H | H | H | H | D | H | H | H | H |
| D1H | D | H | H | H | H | H | D | H | H | H |
| D1I | D | H | H | H | H | H | H | D | H | H |
| D1J | D | H | H | H | H | H | H | H | D | H |
| D1K | D | H | H | H | H | H | H | H | H | D |
| D1L | D | D | D | D | H | H | H | H | H | H |
| D1M | H | D | D | H | H | H | H | H | H | H |
| D1N | H | D | H | D | H | H | H | H | H | H |
| D1O | H | D | H | H | D | H | H | H | H | H |
| D1P | H | D | H | H | H | D | H | H | H | H |
| D1Q | H | D | H | H | H | H | D | H | H | H |
| D1R | H | D | H | H | H | H | H | D | H | H |
| D1S | H | D | H | H | H | H | H | H | D | H |
| D1T | H | D | H | H | H | H | H | H | H | D |
| D1U | D | H | D | D | H | H | H | H | H | H |
| D1V | D | D | H | D | H | H | H | H | H | H |

### Example 02

Formula D2 is the generic formula of deuterated Example 2, wherein Y₁, Y₂, Y₃, Y₄, Y₅, Y₆, Y₇, Y₈, Y₉, and Y₁₀ are each independently H or D. Table 5 shows some examples of deuterated compounds based on the most likely metabolized sites that can be applied to the compounds predicted by MetaSite.

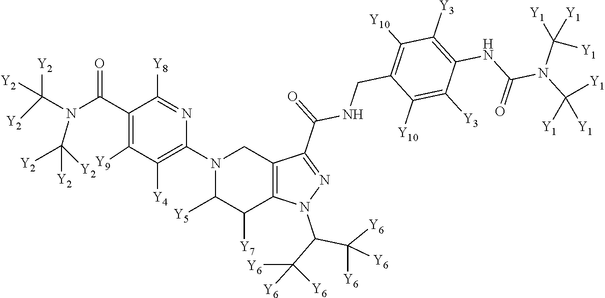

TABLE 5

| Deuteration | | | | | | | | | | |
|---|---|---|---|---|---|---|---|---|---|---|
| Example | Y₁ | Y₂ | Y₃ | Y₄ | Y₅ | Y₆ | Y₇ | Y₈ | Y₉ | Y₁₀ |
| D2A | D | H | H | H | H | H | H | H | H | H |
| D2B | D | D | H | H | H | H | H | H | H | H |
| D2C | D | D | D | H | H | H | H | H | H | H |
| D2D | D | H | D | H | H | H | H | H | H | H |
| D2E | D | H | H | D | H | H | H | H | H | H |
| D2F | D | H | H | H | D | H | H | H | H | H |
| D2G | D | H | H | H | H | D | H | H | H | H |
| D2H | D | H | H | H | H | H | D | H | H | H |
| D2I | D | H | H | H | H | H | H | D | H | H |
| D2J | D | H | H | H | H | H | H | H | D | H |
| D2K | D | H | H | H | H | H | H | H | H | D |
| D2L | D | D | D | D | H | H | H | H | H | H |
| D2M | H | D | D | H | H | H | H | H | H | H |
| D2N | H | D | H | D | H | H | H | H | H | H |
| D2O | H | D | H | H | D | H | H | H | H | H |
| D2P | H | D | H | H | H | D | H | H | H | H |
| D2Q | H | D | H | H | H | H | D | H | H | H |
| D2R | H | D | H | H | H | H | H | D | H | H |
| D2S | H | D | H | H | H | H | H | H | D | H |
| D2T | H | D | H | H | H | H | H | H | H | D |
| D2U | D | H | D | D | H | H | H | H | H | H |
| D2V | D | D | H | D | H | H | H | H | H | H |

### Example 06

Formula D6 is the generic formula of deuterated Example 6, wherein Y₁, Y₂, Y₃, Y₄, Y₅, Y₆, Y₇, Y₈, Y₉, and Y₁₀ are each independently H or D. Table 6 shows some examples of deuterated compounds based on the most likely metabolized sites that can be applied to the compounds predicted by MetaSite.

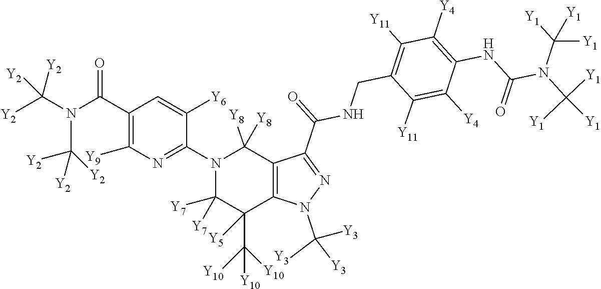

TABLE 6

| Deuteration | | | Y₃ and/ | | | | | | | |
|---|---|---|---|---|---|---|---|---|---|---|
| Example | Y₁ | Y₂ | or Y₁₁ | Y₄ | Y₅ | Y₆ | Y₇ | Y₈ | Y₉ | Y₁₀ |
| D6A | D | H | H | H | H | H | H | H | H | H |
| D6B | D | D | H | H | H | H | H | H | H | H |
| D6C | D | D | D | H | H | H | H | H | H | H |
| D6D | D | H | D | H | H | H | H | H | H | H |
| D6E | D | H | H | D | H | H | H | H | H | H |
| D6F | D | H | H | H | D | H | H | H | H | H |
| D6G | D | H | H | H | H | D | H | H | H | H |
| D6H | D | H | H | H | H | H | D | H | H | H |
| D6I | D | H | H | H | H | H | H | D | H | H |
| D6J | D | H | H | H | H | H | H | H | D | H |
| D6K | D | H | H | H | H | H | H | H | H | D |
| D6L | D | D | D | D | H | H | H | H | H | H |
| D6M | H | D | D | H | H | H | H | H | H | H |
| D6N | H | D | H | D | H | H | H | H | H | H |
| D6O | H | D | H | H | D | H | H | H | H | H |
| D6P | H | D | H | H | H | D | H | H | H | H |
| D6Q | H | D | H | H | H | H | D | H | H | H |
| D6R | H | D | H | H | H | H | H | D | H | H |
| D6S | H | D | H | H | H | H | H | H | D | H |
| D6T | H | D | H | H | H | H | H | H | H | D |
| D6U | D | H | D | D | H | H | H | H | H | H |
| D6V | D | D | H | D | H | H | H | H | H | H |

### Example 031

Formula D31 is the generic formula of deuterated Example 31, wherein Y₁, Y₂, Y₃, Y₄, Y₅, Y₆, Y₇, Y₈, Y₉, and Y₁₀ are each independently H or D. Table 7 shows some examples of deuterated compounds based on the most likely metabolized sites that can be applied to the compounds predicted by MetaSite.

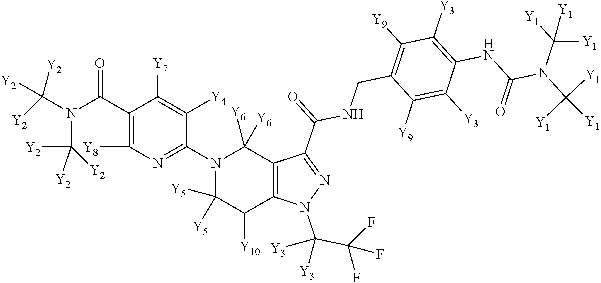

TABLE 7

| Deuteration | | | | | | | | | | |
|---|---|---|---|---|---|---|---|---|---|---|
| Example | Y₁ | Y₂ | Y₃ | Y₄ | Y₅ | Y₆ | Y₇ | Y₈ | Y₉ | Y₁₀ |
| D31A | D | H | H | H | H | H | H | H | H | H |
| D31B | D | D | H | H | H | H | H | H | H | H |
| D31C | D | D | D | H | H | H | H | H | H | H |
| D31D | D | H | D | H | H | H | H | H | H | H |
| D31E | D | H | H | D | H | H | H | H | H | H |
| D31F | D | H | H | H | D | H | H | H | H | H |
| D31G | D | H | H | H | H | D | H | H | H | H |
| D31H | D | H | H | H | H | H | D | H | H | H |
| D31I | D | H | H | H | H | H | H | D | H | H |
| D31J | D | H | H | H | H | H | H | H | D | H |
| D31K | D | H | H | H | H | H | H | H | H | D |
| D31L | D | D | D | D | H | H | H | H | H | H |
| D31M | H | D | D | H | H | H | H | H | H | H |
| D31N | H | D | H | D | H | H | H | H | H | H |
| D31O | H | D | H | H | D | H | H | H | H | H |
| D31P | H | D | H | H | H | D | H | H | H | H |
| D31Q | H | D | H | H | H | H | D | H | H | H |
| D31R | H | D | H | H | H | H | H | D | H | H |
| D31S | H | D | H | H | H | H | H | H | D | H |
| D31T | H | D | H | H | H | H | H | H | H | D |
| D31U | D | H | D | D | H | H | H | H | H | H |
| D31V | D | D | H | D | H | H | H | H | H | H |

### Example 033

Formula D33 is the generic formula of deuterated Example 33, wherein Y₁, Y₂, Y₃, Y₄, Y₅, Y₆, Y₇, Y₈, Y₉, and Y₁₀ are each independently H or D. Table 8 shows some examples of deuterated compounds based on the most likely metabolized sites that can be applied to the compounds predicted by MetaSite.

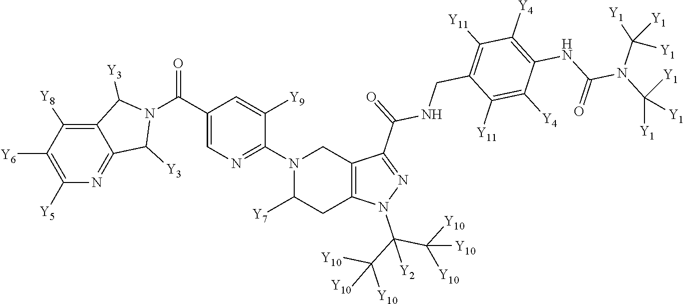

TABLE 8

| Deuteration | | | | Y₄ and/ | | | | | | |
|---|---|---|---|---|---|---|---|---|---|---|
| Example | Y₁ | Y₂ | Y₃ | or Y₁₁ | Y₅ | Y₆ | Y₇ | Y₈ | Y₉ | Y₁₀ |
| D33A | D | H | H | H | H | H | H | H | H | H |
| D33B | D | D | H | H | H | H | H | H | H | H |
| D33C | D | D | D | H | H | H | H | H | H | H |
| D33D | D | H | D | H | H | H | H | H | H | H |
| D33E | D | H | H | D | H | H | H | H | H | H |
| D33F | D | H | H | H | D | H | H | H | H | H |
| D33G | D | H | H | H | H | D | H | H | H | H |
| D33H | D | H | H | H | H | H | D | H | H | H |
| D33I | D | H | H | H | H | H | H | D | H | H |
| D33J | D | H | H | H | H | H | H | H | D | H |
| D33K | D | H | H | H | H | H | H | H | H | D |
| D33L | H | D | D | D | H | H | H | H | H | H |
| D33M | H | D | D | H | H | H | H | H | H | H |
| D33N | H | D | H | D | H | H | H | H | H | H |
| D33O | H | D | H | H | D | H | H | H | H | H |
| D33P | H | D | H | H | H | D | H | H | H | H |
| D33Q | H | D | H | H | H | H | D | H | H | H |
| D33R | H | D | H | H | H | H | H | D | H | H |
| D33S | H | D | H | H | H | H | H | H | D | H |
| D33T | H | D | H | H | H | H | H | H | H | D |
| D33U | D | H | D | D | H | H | H | H | H | H |
| D33V | D | D | H | D | H | H | H | H | H | H |

### Example 0128

Formula 0128 is the generic formula of deuterated Example 128, wherein Y1, Y2, Y3, Y4, Y5, Y6, Y7, Y8, Y9, and Y10 are each independently H or D. Table 9 shows some examples of deuterated compounds based on the most likely metabolized sites that can be applied to the compounds predicted by MetaSite.

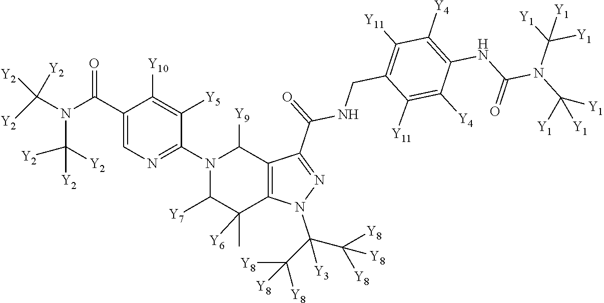

TABLE 9

| Deuteration | | | | Y₄ and/ | | | | | | |
|---|---|---|---|---|---|---|---|---|---|---|
| Example | Y₁ | Y₂ | Y₃ | or Y₁₁ | Y₅ | Y₆ | Y₇ | Y₈ | Y₉ | Y₁₀ |
| D128A | D | H | H | H | H | H | H | H | H | H |
| D128B | D | D | H | H | H | H | H | H | H | H |
| D128C | D | D | D | H | H | H | H | H | H | H |
| D128D | D | H | D | H | H | H | H | H | H | H |
| D128E | D | H | H | D | H | H | H | H | H | H |
| D128F | D | H | H | H | D | H | H | H | H | H |
| D128G | D | H | H | H | H | D | H | H | H | H |
| D128H | D | H | H | H | H | H | D | H | H | H |
| D128I | D | H | H | H | H | H | H | D | H | H |
| D128J | D | H | H | H | H | H | H | H | D | H |
| D128K | D | H | H | H | H | H | H | H | H | D |
| D128L | D | D | D | D | H | H | H | H | H | H |
| D128M | H | D | D | H | H | H | H | H | H | H |
| D128N | H | D | H | D | H | H | H | H | H | H |
| D128O | H | D | H | H | D | H | H | H | H | H |
| D128P | H | D | H | H | H | D | H | H | H | H |
| D128Q | H | D | H | H | H | H | D | H | H | H |
| D128R | H | D | H | H | H | H | H | D | H | H |
| D128S | H | D | H | H | H | H | H | H | D | H |
| D128T | H | D | H | H | H | H | H | H | H | D |
| D128U | D | H | D | D | H | H | H | H | H | H |
| D128V | D | D | H | D | H | H | H | H | H | H |

### Example 0132

Formula D132 is the generic formula of deuterated Example 132, wherein Y1, Y2, Y3, Y4, Y5, Y6, Y7, Y8, Y9, and Y10 are each independently H or D. Table 10 shows some examples of deuterated compounds based on the most likely metabolized sites that can be applied to the compounds predicted by MetaSite.

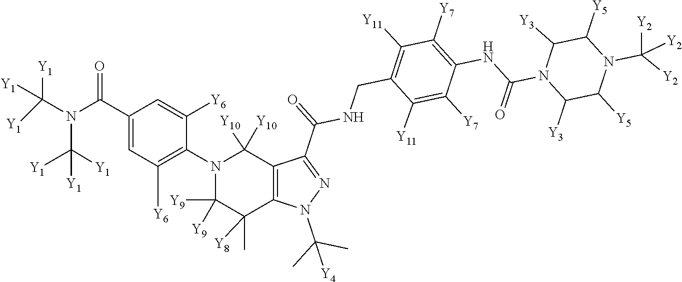

TABLE 10

| Deuteration | | | | | | | Y₇ and/ | | | |
|---|---|---|---|---|---|---|---|---|---|---|
| Example | Y₁ | Y₂ | Y₃ | Y₄ | Y₅ | Y₆ | or Y₁₁ | Y₈ | Y₉ | Y₁₀ |
| D132A | D | H | H | H | H | H | H | H | H | H |
| D132B | D | D | H | H | H | H | H | H | H | H |
| D132C | D | D | D | H | H | H | H | H | H | H |
| D132D | D | H | D | H | H | H | H | H | H | H |
| D132E | D | H | H | D | H | H | H | H | H | H |
| D132F | D | H | H | H | D | H | H | H | H | H |
| D132G | D | H | H | H | H | D | H | H | H | H |
| D132H | D | H | H | H | H | H | D | H | H | H |
| D132I | D | H | H | H | H | H | H | D | H | H |
| D132J | D | H | H | H | H | H | H | H | D | H |
| D132K | D | H | H | H | H | H | H | H | H | D |
| D132L | D | D | D | D | H | H | H | H | H | H |
| D132M | H | D | D | H | H | H | H | H | H | H |
| D132N | H | D | H | D | H | H | H | H | H | H |
| D132O | H | D | H | H | D | H | H | H | H | H |
| D132P | H | D | H | H | H | D | H | H | H | H |
| D132Q | H | D | H | H | H | H | D | H | H | H |
| D132R | H | D | H | H | H | H | H | D | H | H |
| D132S | H | D | H | H | H | H | H | H | D | H |
| D132T | H | D | H | H | H | H | H | H | H | D |
| D132U | D | H | D | D | H | H | H | H | H | H |
| D132V | D | D | H | D | H | H | H | H | H | H |

It will be apparent to those skilled in the art that various modifications and variations may be made in the present disclosure without departing from the scope or spirit of the disclosure.

Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

LENGTHY TABLES
The patent application contains a lengthy table section. A copy of the table is available in electronic form from the USPTO web site (https://seqdata.uspto.gov/?pageRequest=docDetail&DocID=US20250320213A1). An electronic copy of the table will also be available from the USPTO upon request and payment of the fee set forth in 37 CFR 1.19(b)(3).

We claim:

1. A compound of Formula I, or a pharmaceutically acceptable salt thereof: 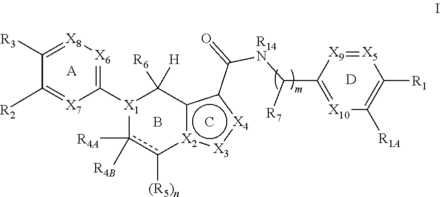
wherein
X₁ is CH, CNH₂, or N;
X₂ is C or N;
X₃ is CR₁₃, N, NR₁₃, O, or S;
X₄ is CH, N, or NR₁₃;
X₅ is CR_{1B} or N;
X₆ is CH or N;
X₇ is CH, N, or CF;
X₈ is CH or N;
X₉ is CR_{1B} or N;
X₁₀ is CR_{1B} or N;
R₁ is —NHR₃, —OH, —C₂₋₅ heterocycle, or —C₁₋₃ alkyl; wherein the —C₂₋₅ heterocycle of R₁ is optionally substituted with one, two, or three of —C₁₋₃ alkyl, —C₁₋₃ oxoalkyl, —C₃₋₅ heterocycloalkyl, —C₃₋₅ hydroxyheterocycloalkyl, —C₃₋₅ aminoheterocycloalkyl, or oxo; wherein the —C₁₋₃ alkyl of R₁ is optionally substituted with one, two, or three of oxo, —NR_{10A}R₁₁, or —NR₁₀R₁₁;
R_{1A} is H, halogen, or —CH₃; or
R₁ and R_{1A} form a C₅₋₇ heterocycloalkyl fused to Ring D or a C₅₋₇ heteroaryl fused to Ring D; wherein the C₅₋₇ heterocycloalkyl or the C₅₋₇ heteroaryl is optionally substituted with oxo;
each R_{1B} independently is H, —CH₃, F, Cl, or methoxy;
R₂ is H, —C₁₋₃ alkyl, —C₁₋₃ alkoxy, —C₁₋₃ fluoroalkyl, or halogen;
R₃ is —C₁₋₃ alkyl, —C₂₋₁₀ heterocycle, —P(═O)(CH₃)₂, —S(═O)CH₃, —NH—S(═O)₂CH₃, or —NH—C(═O)—C₁₋₃ alkyl; wherein the —C₁₋₃ alkyl of R₃ is optionally substituted with one, two, or three of oxo, halogen, —C₃₋₆ cycloalkyl, —OH, —NR₁₂R_{12A}, or cyano; wherein the —C₂₋₁₀ heterocycle is optionally substituted with one, two, or three of —OH, halogen, —C₁₋₃ hydroxyalkyl, —C₁₋₃ alkoxy, —C₁₋₃ alkyl, —C₁₋₃ fluoroalkyl, cyclopropyl, or oxo; or
R₂ and R₃ form a C₃₋₆ heterocycle fused to Ring A optionally substituted with one, two, or three of —C₁₋₃ alkyl, —C₂₋₃ oxoalkyl, or oxo;
R_{4A} is H, —OH, —C₁₋₃ fluoroalkyl, or —C₁₋₃ alkyl;
R_{4B} is H or is absent; or
R_{4A} and R_{4B} form cyclopropyl;
each R₅ independently is H, halogen, —OH, cyclopropyl, —C₁₋₃ fluoroalkyl, or —C₁₋₃ alkyl; or two R₅ groups form cyclopropyl;
R₆ is H, —OH, or —C₁₋₃ alkyl; or
R_{4A} and R₆ or one R₅ and R₆ form a C₁₋₃ alkyl bridge or a C₁₋₃ heteroalkyl bridge; or
R_{4B} and one R₅ form a C₃₋₅ cycloalkyl fused to Ring B;
R₇ is H, —C₁₋₃ alkyl, or —C₁₋₃ hydroxyalkyl;
R₈ is H, —C₁₋₃ alkyl, —SO₂CH₃, or a —C₃₋₄ heterocycle; wherein the —C₁₋₃ alkyl of R₃ is optionally substituted with one, two, or three of oxo, —C₃₋₉ heterocycloalkyl, —C₁₋₃ alkoxy, cyanoimine, or —NR₉R₁₀; wherein the —C₃₋₄ heterocycle of R₈ is optionally substituted with one, two, or three of oxo, halogen, —C₀₋₁ alkylene-NR₁₀R₁₁, —OH, —C₁₋₃ hydroxyalkyl, —C₁₋₃ alkoxy optionally substituted with —NR₁₀R₁₁, —C₁₋₃ alkyl-C₁₋₃ alkoxy, —C₁₋₃ oxoalkyl, or —C₁₋₃ alkyl; wherein the —C₃₋₉ heterocycloalkyl of R₃ is optionally substituted with one, two, or three of —C₁₋₃ alkyl, —OH, —C₁₋₃ hydroxyalkyl, —O—C₁₋₃ hydroxyalkyl, —C₁₋₃ alkoxy, —C₁₋₃ alkyl-C₁₋₃ alkoxy, halogen, —C₁₋₃ fluoroalkyl, —C₁₋₃ fluoroalkoxy, cyano, —C₁₋₃ cyanoalkyl, —C₀₋₁ alkylene-C₃₋₅ heterocycloalkyl, —O—C₃₋₅ heterocycloalkyl, or —C₀₋₁ alkylene-NR₁₀R₁₁;
R₉ is H, —C₁₋₄ alkyl, —C₁₋₄ hydroxyalkyl, —C₁₋₃ fluoroalkyl, —C₃₋₅ cycloalkyl, -methylene-phenylene-NH—C(═O)—NR₁₀R₁₁, or —C₃₋₅ heterocycloalkyl; wherein the —C₁₋₄ alkyl of R₉ is optionally substituted with one, two, or three of —C₃₋₅ cycloalkyl, —C₃₋₅ heterocycloalkyl, or methoxy; wherein the —C₃₋₅ cycloalkyl of R₉ is optionally substituted with one or two of —OH or —C₁₋₃ alkyl;
each R₁₀ independently is H or —C₁₋₄ alkyl;
R_{10A} is —C₀₋₁ alkylene-C₃₋₅ heterocycloalkyl optionally substituted with —C₁₋₃ alkyl;
each R₁₁ independently is H or —C₁₋₃ alkyl;
R₁₂ is H, —C₁₋₃ alkyl, —C₁₋₃ hydroxyalkyl, —(CH₂)₀₋₂—C₃₋₅ heterocycloalkyl, —(CH₂)₁₋₂—O—C₃₋₅ heterocycloalkyl, -methylene-C(═O)—NR₁₀R₁₁, or -methylene-phenylene-NH—C(═O)—NR₁₀R₁₁;
wherein the —(CH₂)₀₋₂—C₃₋₅ heterocycloalkyl or the —(CH₂)₁₋₂—O—C₃₋₅ heterocycloalkyl of R₁₂ is optionally substituted with —C₁₋₄ alkyl, —C₁₋₃ alkyl-C₁₋₃ alkoxy, —(CH₂)₀₋₁-phenyl, halogen, —C₁₋₃ fluoroalkyl, or —(CH₂)₀₋₁—C₃₋₅ heterocycloalkyl; wherein the —C₁₋₃ alkyl of R₁₂ is optionally substituted with one, two, or three of oxo or NR₁₀R₁₁;
R_{12A} is H or —C₁₋₄ alkyl; or
R₁₂ and R_{12A} form a C₃₋₁₀ heterocycle optionally substituted with one, two, or three of —OH, —C₁₋₃ alkyl, oxo, halogen, —C₂₋₃ oxoalkyl, —C₁₋₃ alkoxy, —C₁₋₃ hydroxyalkyl, —C₁₋₃ alkyl-C₁₋₃ alkoxy, cyano, —C₃₋₆ cycloalkyl, —S(═O)₂CH₃, —S(═O)₂CH₂CH₃, —C(═O)—NR₁₀R₁₁, or —NHC(═O)CH₃;
R₁₃ is H, —C₁₋₆ alkyl, —C₁₋₄ hydroxyalkyl, —C₁₋₃ fluoroalkyl, —C₁₋₃ alkyl-C₁₋₃ alkoxy, —C₁₋₄ cyanoalkyl, —C₀₋₁ alkylene-C₃₋₅ cycloalkyl, —C₂₋₄ oxoalkyl, or —C₀₋₁ alkylene-C₃₋₅ heterocycloalkyl; wherein the —C₁₋₆ alkyl of R₁₃ is optionally substituted with one, two, or three of oxo, fluoro, —NH₂, C₃₋₆ cycloalkyl, or methoxy; wherein the —C₃₋₅ cycloalkyl or the —C₃₋₆ cycloalkyl of R₁₃ is optionally substituted with one, two, or three of —C₁₋₃ alkyl, —C₁₋₃ fluoroalkyl, or halogen;
R₁₄ is H; or
X₄ is NR₁₃ and R₁₃ and R₁₄ form a C₄₋₅ heterocycle fused to Ring C;
n is 1 or 2;
m is 0 or 1;
wherein if X₃ is CR₁₃ or NR₁₃ then X₄ is CH or N, and if X₄ is NR₁₃ then X₃ is N, O, or S;
wherein the respective heterocycles each independently include 1 to 4 heteroatoms including at least one of N, O, or S.

2. The compound of claim 1, or a pharmaceutically acceptable salt thereof, wherein R₃ is —C₁₋₃ alkyl or —C₂₋₁₀ heterocycle; wherein the —C₂₋₁₀ heterocycle is —C₂₋₅ heteroaryl; wherein the —C₁₋₃ alkyl of R₃ is optionally substituted with one, two, or three of oxo or —NR₁₂R_{12A}; wherein the —C₂₋₅ heterocycle of R₃ is optionally substituted with —C₁₋₃ alkyl.

3. The compound of claim 1, or a pharmaceutically acceptable salt thereof, wherein R₁ is —NHR₃; and R₃ is —C₁₋₃ alkyl substituted with one, two, or three of oxo or —NR₉R₁₀.

4. The compound of claim 1, or a pharmaceutically acceptable salt thereof, wherein R₂ is H; and R₃ is —C₁₋₃ alkyl substituted with oxo and —NR₁₂R_{12A}.

5. The compound of claim 1, or a pharmaceutically acceptable salt thereof, wherein at least one of: X₅ is CH; X₇ is CH or N; X₈ is CH; R₁ is —NHR₈, —C₂₋₅ heterocycle, or —C₁₋₃ alkyl; R_{4A} is H or —C₁₋₃ alkyl; each R₅ independently is H, halogen, or —C₁₋₃ alkyl; or the two R₅ groups form cyclopropyl; R₆ is H or —C₁₋₃ alkyl; or R₇ is H or —C₁₋₃ alkyl.

6. The compound of claim 1, or a pharmaceutically acceptable salt thereof, wherein R₃ is —C₁₋₃ alkyl substituted with oxo and —NR₁₂R_{12A} and R₁ is —NHR₈ or —C₁₋₃ alkyl substituted with oxo and —NR₁₀R₁₁.

7. The compound of claim 1, or a pharmaceutically acceptable salt thereof, wherein X₃ is CR₁₃ and X₄ is CH or N.

8. The compound of claim 1, or a pharmaceutically acceptable salt thereof, wherein R₁ is —NHR₈; wherein R₈ is —C₁₋₃ alkyl or a —C₃₋₄ heterocycle; wherein the —C₁₋₃ alkyl of R₈ is substituted with oxo and —NR₉R₁₀; wherein the —C₃₋₄ heterocycle is substituted with one, two, or three of oxo or —C₁₋₃ alkyl.

9. The compound of claim 1, or a pharmaceutically acceptable salt thereof, wherein X₁ is N, X₃ is N or NR₁₃, and X₄ is N.

10. The compound of claim 1, or a pharmaceutically acceptable salt thereof, wherein the —C₂₋₁₀ heterocycle of R₃ is a —C₂₋₅ heterocycle and R₃ is optionally substituted with one, two, or three of —C₁₋₃ alkyl.

11. The compound of claim 1, or a pharmaceutically acceptable salt thereof, wherein at least one hydrogen is deuterium.

12. The compound of claim 11, or a pharmaceutically acceptable salt thereof, wherein R₁ is

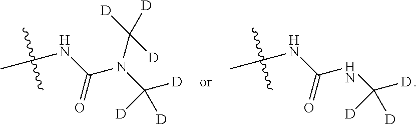 [SMILES: *NC(=O)N(C([2H])([2H])[2H])C([2H])([2H])[2H] ; *NC(=O)NC([2H])([2H])[2H]]

13. The compound of claim 11, or a pharmaceutically acceptable salt thereof, wherein R_{1A} is deuterium; X₅, X₉, and X₁₀ are each CR_{1B}; and the R_{1B} of each of X₅, X₉, and X₁₀ is deuterium.

14. A pharmaceutical composition comprising the compound of claim 1, or a pharmaceutically acceptable salt thereof, and at least one pharmaceutically acceptable excipient.

15. A method for treating atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa comprising administering to a subject in need thereof a therapeutically effective amount of the compound of claim 1, or a pharmaceutically acceptable salt thereof.

16. A method for treating a dermatological condition or a respiratory condition comprising administering to a subject in need thereof a therapeutically effective amount of the compound of claim 1, or a pharmaceutically acceptable salt thereof.

17. A compound or a pharmaceutically acceptable salt thereof; wherein the compound is one of:
N-(4-(3,3-dimethylureido)benzyl)-1-isopropyl-5-(5-(2-oxopyrrolidin-1-yl)pyridin-2-yl)-4,5,6,7-tetrahydro-1H-pyrazolo[4,3-c]pyridine-3-carboxamide;
5-(5-(Dimethylcarbamoyl)pyridin-2-yl)-N-(4-(3,3-dimethylureido)benzyl)-1-isopropyl-4,5,6,7-tetrahydro-1H-pyrazolo[4,3-c]pyridine-3-carboxamide;
5-(5-(6,7-dihydro-5H-pyrrolo[3,4-b]pyridine-6-carbonyl)pyridin-2-yl)-N-(4-(3,3-dimethylureido)benzyl)-1-isopropyl-4,5,6,7-tetrahydro-1H-pyrazolo[4,3-c]pyridine-3-carboxamide;
5-(5-(Dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide;
(S)-5-(5-(dimethylcarbamoyl)pyridin-2-yl)-N-(4-(3,3-dimethylureido)benzyl)-1-isopropyl-7-methyl-4,5,6,7-tetrahydro-1H-pyrazolo[4,3-c]pyridine-3-carboxamide;
(S)-5-(4-(dimethylcarbamoyl)phenyl)-1-isopropyl-7-methyl-N-(4-(4-methylpiperazine-1-carboxamido)benzyl)-4,5,6,7-tetrahydro-1H-pyrazolo[4,3-c]pyridine-3-carboxamide; or
5-(5-(dimethylcarbamoyl)pyridin-2-yl)-N-(4-(3,3-dimethylureido)benzyl)-1-(2,2,2-trifluoroethyl)-4,5,6,7-tetrahydro-1H-pyrazolo[4,3-c]pyridine-3-carboxamide.

18. A compound, wherein the compound is one of:
N-(4-(3,3-dimethylureido)benzyl)-1-isopropyl-5-(5-(2-oxopyrrolidin-1-yl)pyridin-2-yl)-4,5,6,7-tetrahydro-1H-pyrazolo[4,3-c]pyridine-3-carboxamide;
5-(5-(Dimethylcarbamoyl)pyridin-2-yl)-N-(4-(3,3-dimethylureido)benzyl)-1-isopropyl-4,5,6,7-tetrahydro-1H-pyrazolo[4,3-c]pyridine-3-carboxamide;
5-(5-(6,7-dihydro-5H-pyrrolo[3,4-b]pyridine-6-carbonyl)pyridin-2-yl)-N-(4-(3,3-dimethylureido)benzyl)-1-isopropyl-4,5,6,7-tetrahydro-1H-pyrazolo[4,3-c]pyridine-3-carboxamide;
5-(5-(Dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide;
(S)-5-(5-(dimethylcarbamoyl)pyridin-2-yl)-N-(4-(3,3-dimethylureido)benzyl)-1-isopropyl-7-methyl-4,5,6,7-tetrahydro-1H-pyrazolo[4,3-c]pyridine-3-carboxamide;
(S)-5-(4-(dimethylcarbamoyl)phenyl)-1-isopropyl-7-methyl-N-(4-(4-methylpiperazine-1-carboxamido)benzyl)-4,5,6,7-tetrahydro-1H-pyrazolo[4,3-c]pyridine-3-carboxamide; or
5-(5-(dimethylcarbamoyl)pyridin-2-yl)-N-(4-(3,3-dimethylureido)benzyl)-1-(2,2,2-trifluoroethyl)-4,5,6,7-tetrahydro-1H-pyrazolo[4,3-c]pyridine-3-carboxamide.

19. A compound or a pharmaceutically acceptable salt thereof; wherein the compound is

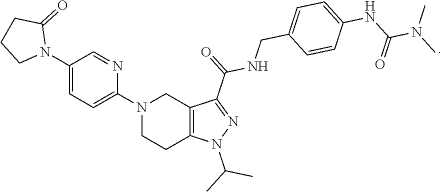 [SMILES: CC(C)n1nc(C(=O)NCc2ccc(NC(=O)N(C)C)cc2)c2c1CCN(c1ccc(N3CCCC3=O)cn1)C2]

20. A compound or a pharmaceutically acceptable salt thereof; wherein the compound is

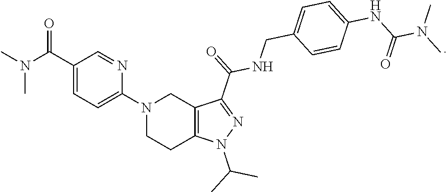

21. A compound or a pharmaceutically acceptable salt thereof; wherein the compound is

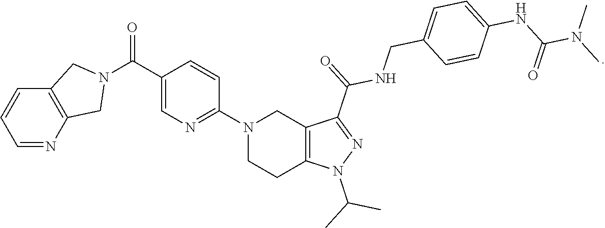 [SMILES: CC(C)n1nc(C(=O)NCc2ccc(NC(=O)N(C)C)cc2)c2c1CCN(c1ccc(C(=O)N3Cc4cccnc4C3)cn1)C2]

22. A compound or a pharmaceutically acceptable salt thereof; wherein the compound is

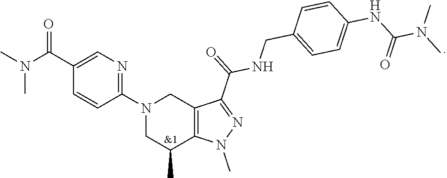

23. A compound or a pharmaceutically acceptable salt thereof; wherein the compound is

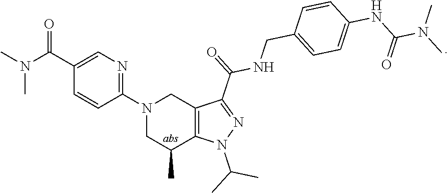

24. A compound or a pharmaceutically acceptable salt thereof; wherein the compound is

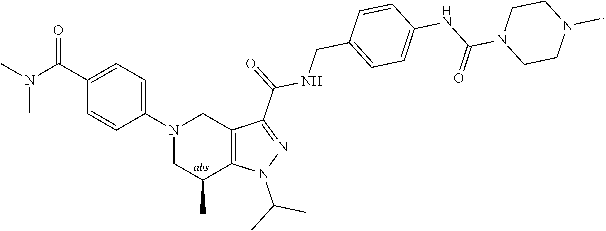

25. A compound or a pharmaceutically acceptable salt thereof; wherein the compound is [chemical structure]
